(19) United States
(12) Patent Application Publication
(10) Pub. No.: US 2024/0279658 A1
(43) Pub. Date: Aug. 22, 2024

(54) DOUBLE STRANDED OLIGONUCLEOTIDE COMPOSITIONS AND METHODS RELATING THERETO

(71) Applicant: WAVE LIFE SCIENCES LTD., Singapore (SG)

(72) Inventors: Chandra Vargeese, Schwenksville, PA (US); Naoki IWAMOTO, Boston, MA (US); Wei LIU, Malden, MA (US); Ngoc Dang Khoa LUU, Charlestown, MA (US); Pachamuthu KANDASAMY, Lexington, MA (US); Subramanian MARAPPAN, Acton, MA (US); Snehlata TRIPATHI, Waltham, MA (US)

(73) Assignee: WAVE LIFE SCIENCES LTD., Singapore (SG)

(21) Appl. No.: 18/612,623

(22) Filed: Mar. 21, 2024

Related U.S. Application Data
(63) Continuation of application No. PCT/US2022/044296, filed on Sep. 21, 2022.
(60) Provisional application No. 63/246,756, filed on Sep. 21, 2021.

(51) Int. Cl. C12N 15/113

(52) U.S. Cl. CPC C12N 15/113; C12N 2310/14; C12N 2310/314; C12N 2310/315; C12N 2310/321; C12N 2310/322; C12N 2310/332; C12N 2310/351

(57) ABSTRACT

The present disclosure provides double stranded oligonucleotides, compositions, and methods relating thereto. The present disclosure encompasses the recognition that structural elements of double stranded oligonucleotides, such as base sequence, chemical modifications (e.g., modifications of sugar, base, and/or internucleotidic linkages) or patterns thereof, and/or stereochemistry (e.g., stereochemistry of backbone chiral centers (chiral internucleotidic linkages)), and/or patterns thereof, can have significant impact on oligonucleotide properties and activities, e.g., RNA interference (RNAi) activity, stability, delivery, etc. The present disclosure also provides methods for treatment of diseases using provided double stranded oligonucleotide compositions, for example, in RNA interference.

### CROSS-REFERENCE TO RELATED APPLICATIONS

This application is a continuation of International Application No. PCT/US2022/044296, filed Sep. 21, 2022, which claims priority to U.S. Provisional Patent Application Ser. No. 63/246,756, filed Sep. 21, 2021, the contents of which are incorporated by reference in their entirety.

### SEQUENCE LISTING

The present application contains a Sequence Listing which has been submitted via EFS-Web and is hereby incorporated by reference in its entirety. Said Sequence Listing, created on Mar. 21, 2024, is named 0882900160.xml and is 958,306 bytes in size.

### BACKGROUND

Gene-targeting oligonucleotides are useful in various applications, e.g., therapeutic, diagnostic, research and nanomaterials applications. The use of naturally-occurring nucleic acids (e.g., unmodified DNA or RNA) in such applications can be limited by, for example, their susceptibility to endo- and exo-nucleases. As such, various synthetic counterparts have been developed to circumvent these shortcomings. These include synthetic oligonucleotides that contain chemical modifications, e.g., base modifications, sugar modifications, backbone modifications. There remains, however, a need in the art for double-stranded (ds) oligonucleotides with improved properties for use in connection with the above-described applications.

### SUMMARY

The present disclosure is directed, in part, to the recognition that controlling structural elements of the oligonucleotides of a double-stranded (ds) oligonucleotide can have a significant impact on the ds oligonucleotide's properties and/or activity. In certain embodiments, such structural elements include one or more of: (1) chemical modifications (e.g., modifications of a sugar, base and/or internucleotidic linkage) and patterns thereof; and (2) alterations in stereochemistry (e.g., stereochemistry of a backbone chiral internucleotidic linkage) and patterns thereof. One or more of such structural elements can, in certain embodiments, be independently present in one or both oligonucleotides of a ds oligonucleotide. In certain embodiments, the properties and/or activities impacted by such structural elements include, but are not limited to, participation in, direction of a decrease in expression, activity or level of a gene or a gene product thereof, mediated, for example, by RNA interference (RNAi interference), RNase H-mediated knockdown, steric hindrance of translation, etc.

In certain embodiments, the present disclosure demonstrates that compositions comprising ds oligonucleotides (e.g., dsRNAi oligonucleotides, also referred to as dsRNAi agents) with controlled structural elements provide unexpected properties and/or activities.

In certain embodiments, the present disclosure encompasses the recognition that stereochemistry, e.g., stereochemistry of backbone chiral centers, can unexpectedly maintain or improve properties of ds oligonucleotides. For example, but not by way of limitation, the instant disclosure relates, in part, to ds oligonucleotides comprising one or more of:
(1) a guide strand comprising backbone phosphorothioate chiral centers in Sp configuration between the 3′ terminal nucleotide and the penultimate (N−1) nucleotide and as between the penultimate (N−1) nucleotide and the immediately upstream, i.e., in the 5′ direction, (N−2) nucleotide;
(2) a guide strand comprising backbone phosphorothioate chiral centers in Rp, Sp, or alternating configurations between the 5′ terminal (+1) nucleotide and the immediately downstream, i.e., in the 3′ direction, (+2) nucleotide and between the +2 nucleotide and the immediately downstream (+3) nucleotide;
(3) a guide strand comprising one or more backbone phosphorothioate chiral centers upstream, i.e., in the 5′ direction, relative to backbone phosphorothioate chiral centers in Sp configuration between the 3′ terminal nucleotide and the penultimate (N−1) nucleotide and as between the penultimate (N−1) nucleotide and the immediately upstream (N−2) nucleotide, where the upstream backbone phosphorothioate chiral centers are in Rp or Sp configuration;
(4) a guide strand comprising one or more backbone phosphorothioate chiral centers in Rp or Sp configuration between the 5′ terminal (+1) nucleotide and the immediately downstream (+2) nucleotide and between the +2 nucleotide and the immediately downstream (+3) nucleotide, as well as between one or both of: (a) the +3 nucleotide and the +4 nucleotide; and (b) the +5 nucleotide and the +6 nucleotide; (5) a passenger strand in combination with one or more of the aforementioned guide strands, comprising one or more backbone chiral centers in Rp or Sp configuration; and
(6) a passenger strand in combination with one or more of the aforementioned guide strands, comprising backbone phosphorothioate chiral centers in the Sp configuration between the 5′ terminal (+1) nucleotide and the immediately downstream, i.e., in the 3′ direction, (+2) nucleotide and between the 3′ terminal nucleotide and the penultimate (N−1) nucleotide;
   wherein the ds oligonucleotide further comprises one or more of:
(1) a guide strand where one or both of the 5′ and 3′ terminal dinucleotides are not linked by a Rp, Sp, or stereorandom non-negatively charged internucleotidic linkages, i.e., the guide strand comprises one or more Rp, Sp, or stereorandom non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N−1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by a Rp, Sp, or stereorandom non-negatively charged internucleotidic linkage. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N−1) nucleotide and the 3′ terminal (N) nucleotide.

In certain embodiments, the present disclosure encompasses the recognition that stereochemistry, e.g., stereochemistry of chiral centers at a 5′ terminal modification of guide strands, can unexpectedly maintain or improve properties of the ds oligonucleotides described herein. For example, but not by way of limitation, the instant disclosure relates, in part, to ds oligonucleotides comprising a guide stranding comprising: (1) a phosphorothioate chiral center in Rp or Sp configuration; (2) an Rp, Sp, or stereorandom non-negatively charged internucleotidic linkage where the 3′ nucleotide of a nucleotide pair linked by an Rp, Sp, or stereorandom non-negatively charged internucleotidic linkage comprises a 2′ modification, e.g., a 2′ F; and (3) a 5′ terminal modification selected from:
(a) 5′ PO modifications, such as, but not limited to:

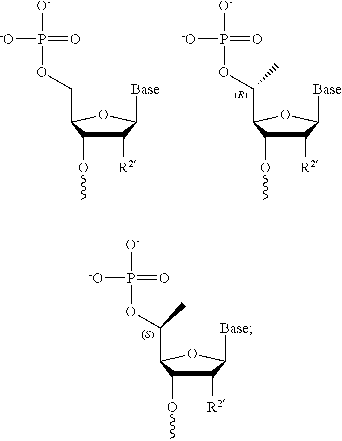

(b) 5′ VP modifications, such as, but not limited to:

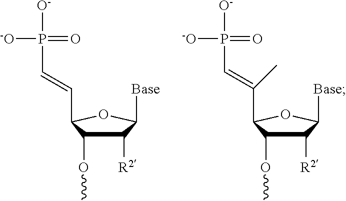

(c) 5′ MeP modifications, such as, but not limited to:

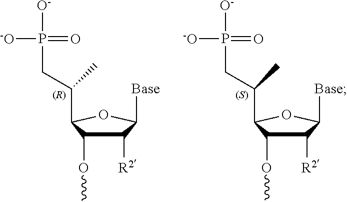

(d) 5′ PN and 5′ Trizole-P modifications, such as, but not limited to:

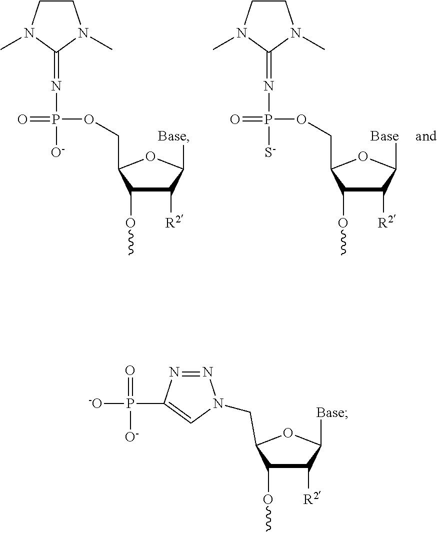

Wherein Base is selected from A, C, G, T, U, abasic and modified nucleobases; R^{2′} is selected from H, OH, O-alkyl, F, MOE, locked nucleic acid (LNA) bridges and bridged nucleic acid (BNA) bridges to the 4′ C, such as, but not limited to:

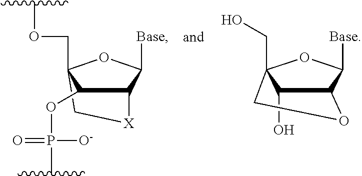

In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In certain other embodiments, the present disclosure encompasses the recognition that stereochemistry, e.g., stereochemistry of chiral centers at the 5′ terminal nucleotide of guide strands, can unexpectedly maintain or improve properties of ds oligonucleotides wherein the guide strand of the ds oligonucleotide also comprises a phosphorothioate chiral center in Rp or Sp configuration. For example, but not by way of limitation, the instant disclosure relates, in part, to ds oligonucleotides comprising a guide stranding comprising: (1) a phosphorothioate chiral center in Rp or Sp configuration; (2) an Rp, Sp, or stereorandom non-negatively charged internucleotidic linkage where the 3′ nucleotide of a nucleotide pair linked by an Rp, Sp, or stereorandom non-negatively charged internucleotidic linkage comprises a 2′ modification, e.g., a 2′ F; and (3) a 5′ terminal modification selected from:
(a) 5′ PO nucleotides, such as, but not limited to:

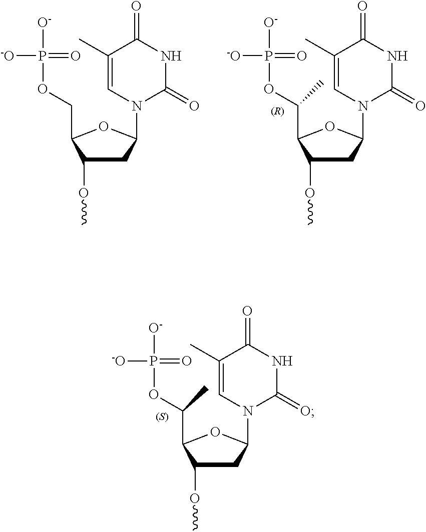

(b) 5′ VP nucleotides, such as, but not limited to:

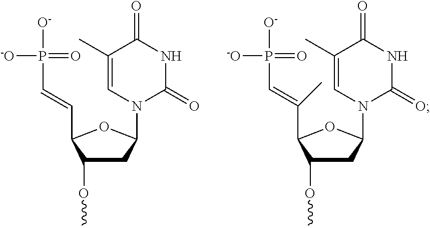

(c) 5′ MeP nucleotides, such as, but not limited to:

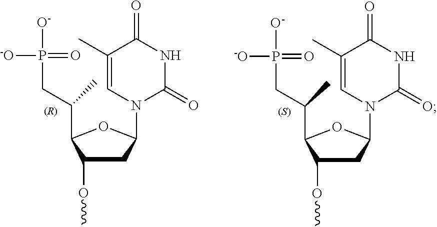

(d) 5′ PN and 5′ Trizole-P nucleotides, such as, but not limited to:

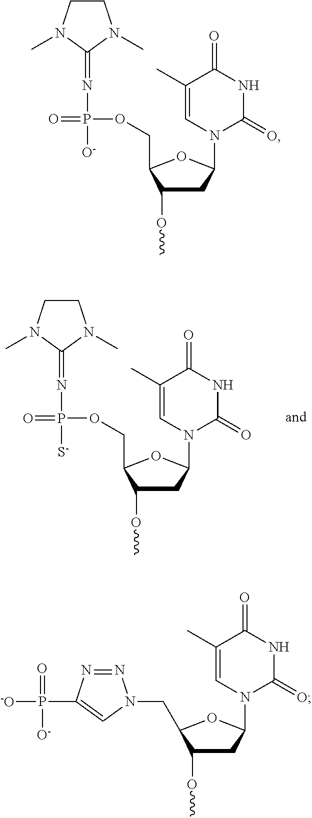

(e) 5′ abasic VP and 5′ abasic MeP nucleotides, such as, but not limited to:

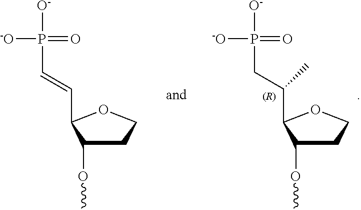

In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In certain embodiments, the present disclosure encompasses the recognition that non-naturally occurring internucleotidic linkages, e.g., neutral internucleotidic linkages, can, in certain embodiments, be used to link one or more molecules to the double-stranded oligonucleotides described herein. In certain embodiments, such linked molecules can facilitate targeting and/or delivery of the double-stranded oligonucleotide. For example, but not limitation, such linked molecules an include lipophilic molecules. In certain embodiments, the linked molecule is a molecule comprising one or more GalNAc moieties. In certain embodiments, the the linked molecule is a receptor. In certain embodiments, the linked molecule is a receptor ligand.

In certain embodiments, the present disclosure provides technologies for incorporating various additional chemical moieties into ds oligonucleotides. In certain embodiments, the present disclosure provides, for example, reagents and methods for introducing additional chemical moieties through nucleobases (e.g., by covalent linkage, optionally via a linker, to a site on a nucleobase).

In certain embodiments, the present disclosure provides technologies, e.g., ds oligonucleotide compositions and methods thereof, that achieve allele-specific suppression, wherein transcripts from one allele of a particular target gene is selectively knocked down relative to at least one other allele of the same gene.

Among other things, the present disclosure provides structural elements, technologies and/or features that can be incorporated into ds oligonucleotides and can impart or tune one or more properties thereof (e.g., relative to an otherwise identical ds oligonucleotide lacking the relevant technology or feature). In certain embodiments, the present disclosure documents that one or more provided technologies and/or features can usefully be incorporated into ds oligonucleotides of various sequences.

In certain embodiments, the present disclosure demonstrates that certain provided structural elements, technologies and/or features are particularly useful for ds oligonucleotides that participate in and/or direct RNAi mechanisms (e.g., RNAi agents). Regardless, however, the teachings of the present disclosure are not limited to ds oligonucleotides that participate in or operate via any particular mechanism. In certain embodiments, the present disclosure pertains to any ds oligonucleotide, useful for any purpose, which operates through any mechanism, and which comprises any sequence, structure or format (or portion thereof) described herein. In certain embodiments, the present disclosure provides a ds oligonucleotide, useful for any purpose, which operates through any mechanism, and which comprises any sequence, structure or format (or portion thereof) described herein, comprising one or more of.
(1) a guide strand comprising backbone phosphorothioate chiral centers in Sp configuration between the 3′ terminal nucleotide and the penultimate (N−1) nucleotide and as between the penultimate (N−1) nucleotide and the immediately upstream, i.e., in the 5′ direction, (N−2) nucleotide;
(2) a guide strand comprising backbone phosphorothioate chiral centers in Rp, Sp, or alternating configurations between the 5′ terminal (+1) nucleotide and the immediately downstream, i.e., in the 3′ direction, (+2) nucleotide and between the +2 nucleotide and the immediately downstream (+3) nucleotide;
(3) a guide strand comprising one or more backbone phosphorothioate chiral centers upstream, i.e., in the 5′ direction, relative to backbone phosphorothioate chiral centers in Sp configuration between the 3′ terminal nucleotide and the penultimate (N−1) nucleotide and as between the penultimate (N−1) nucleotide and the immediately upstream (N−2) nucleotide, where the upstream backbone phosphorothioate chiral centers are in Rp or Sp configuration;
(4) a guide strand comprising one or more backbone phosphorothioate chiral centers in Rp or Sp configuration between the 5′ terminal (+1) nucleotide and the immediately downstream (+2) nucleotide and between the +2 nucleotide and the immediately downstream (+3) nucleotide, as well as between one or both of: (a) the +3 nucleotide and the +4 nucleotide; and (b) the +5 nucleotide and the +6 nucleotide;
(5) a passenger strand in combination with one or more of the aforementioned guide strands, comprising one or more backbone chiral centers in Rp or Sp configuration; and
6) a passenger strand in combination with one or more of the aforementioned guide strands, comprising backbone phosphorothioate chiral centers in the Sp configuration between the 5′ terminal (+1) nucleotide and the immediately downstream, i.e., in the 3′ direction, (+2) nucleotide and between the 3′ terminal nucleotide and the penultimate (N−1) nucleotide;
   wherein the ds oligonucleotide further comprises one or more of:
(1) a guide strand where one or both of the 5′ and 3′ terminal dinucleotides are not linked by non-negatively charged internucleotidic linkages, i.e., the guide strand comprises one more Rp, Sp, or stereorandom non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N−1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N−1) nucleotide and the 3′ terminal (N) nucleotide.

In certain embodiments, the provided ds oligonucleotides may participate in (e.g., direct) RNAi mechanisms. In certain embodiments, provided ds oligonucleotides may participate in RNase H (ribonuclease H) mechanisms. In certain embodiments, provided ds oligonucleotides may act as translational inhibitors (e.g., may provide steric blocks of translation).

In certain embodiments, the guide strand comprises backbone phosphorothioate chiral centers in Sp configuration between the 3′ terminal nucleotide and the penultimate (N−1) nucleotide and as between the penultimate (N−1) nucleotide and the immediately upstream (N−2) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N−1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N−1) nucleotide and the 3′ terminal (N) nucleotide.

In certain embodiments, the guide strand comprises backbone phosphorothioate chiral centers in Rp, Sp, or alternating configurations between the 5′ terminal (+1) nucleotide and the immediately downstream (+2) nucleotide and between the +2 nucleotide and the immediately downstream (+3) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N−1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N−1) nucleotide and the 3′ terminal (N) nucleotide.

In certain embodiments, the guide strand comprises one or more backbone phosphorothioate chiral centers in Rp or Sp configuration upstream of backbone phosphorothioate chiral centers in Sp configuration between the 3′ terminal nucleotide and the penultimate (N−1) nucleotide and as between the penultimate (N−1) nucleotide and the immediately upstream (N−2) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N−1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide; (4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N−1) nucleotide and the 3′ terminal (N) nucleotide.

In certain embodiments, the guide strand comprises one or more Rp, Sp, or stereorandom non-negatively charged internucleotidic linkage occurs between the second (+2) and third (+3) nucleotides, relative to the 5′ terminal nucleotide, of the guide strand and the internucleotidic linkage to the penultimate 3′ (N−1) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N−1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N−1) nucleotide and the 3′ terminal (N) nucleotide.

In certain embodiments, the guide strand comprises backbone phosphorothioate chiral centers in Sp configuration between the 3′ terminal nucleotide and the penultimate (N−1) nucleotide and as between the penultimate (N−1) nucleotide and the immediately upstream (N−2) nucleotide, and one or more of.
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N−1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone phosphorothioate chiral centers in Rp or Sp configuration. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N−1) nucleotide and the 3′ terminal (N) nucleotide.

In certain embodiments, the guide strand comprises backbone phosphorothioate chiral centers in Rp, Sp, or alternating configurations between the 5′ terminal (+1) nucleotide and the immediately downstream (+2) nucleotide and between the +2 nucleotide and the immediately downstream (+3) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N−1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone chiral centers in Rp or Sp configuration. In certain embodiments, the one or more Rp, Sp, or stereorandomnon-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N−1) nucleotide and the 3′ terminal (N) nucleotide.

In certain embodiments, the guide strand comprises one or more backbone phosphorothioate chiral centers in Rp or Sp configuration upstream of backbone chiral centers in Sp configuration between the 3′ terminal nucleotide and the penultimate (N−1) nucleotide and as between the penultimate (N−1) nucleotide and the immediately upstream (N−2) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N−1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone chiral centers in Rp or Sp configuration. In certain embodiments, the one or more Rp, Sp, or stereorandomnon-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N−1) nucleotide and the 3′ terminal (N) nucleotide.

In certain embodiments, the guide strand comprises one or more backbone phosphorothioate chiral centers in Rp or Sp configuration between the 5′ terminal (+1) nucleotide and the immediately downstream (+2) nucleotide and between the (+2) nucleotide and the immediately downstream (+3) nucleotide, as well as between one or both of (a) the (+3) nucleotide and the (+4) nucleotide; and (b) the (+5) nucleotide and the (+6) nucleotide, and one or more of
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N−1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone chiral centers in Rp or Sp configuration. In certain embodiments, the one or more Rp, Sp, or stereorandomnon-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N−1) nucleotide and the 3′ terminal (N) nucleotide.

In certain embodiments, the guide strand comprises one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N−1) nucleotide of the guide strand, where N is the 3′ terminal nucleotide, a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone chiral centers in Rp or Sp configuration. In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N−1) nucleotide and the 3′ terminal (N) nucleotide.

In certain embodiments, the guide strand comprises backbone phosphorothioate chiral centers in Sp configuration between the 3′ terminal nucleotide and the penultimate (N−1) nucleotide and as between the penultimate (N−1) nucleotide and the immediately upstream (N−2) nucleotide, a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49 and one or more backbone chiral centers in Rp or Sp configuration. In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N−1) nucleotide and the 3′ terminal (N) nucleotide.

In certain embodiments, the guide strand comprises backbone phosphorothioate chiral centers in Rp, Sp, or alternating configurations between the 5′ terminal (+1) nucleotide and the immediately downstream (+2) nucleotide and between the +2 nucleotide and the immediately downstream (+3) nucleotide, a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49 and one or more backbone chiral centers in Rp or Sp configuration. In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N−1) nucleotide and the 3′ terminal (N) nucleotide.

In certain embodiments, the guide strand comprises one or more backbone phosphorothioate chiral centers in Rp or Sp configuration upstream of backbone phosphorothioate chiral centers in Sp configuration between the 3′ terminal nucleotide and the penultimate (N−1) nucleotide and as between the penultimate (N−1) nucleotide and the immediately upstream (N−2) nucleotide, a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49 and one or more backbone chiral centers in Rp or Sp configuration. In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N−1) nucleotide and the 3′ terminal (N) nucleotide.

In certain embodiments, the guide strand comprises one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N−1) nucleotide of the guide strand, where N is the 3′ terminal nucleotide, a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49 and one or more backbone chiral centers in Rp or Sp configuration. In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N−1) nucleotide and the 3′ terminal (N) nucleotide.

In certain embodiments, provided ds oligonucleotides may participate in exon skipping mechanisms. In certain embodiments, provided ds oligonucleotides may be aptamers. In certain embodiments, provided ds oligonucleotides may bind to and inhibit the function of a protein, small molecule, nucleic acid or cell. In certain embodiments, provided ds oligonucleotides may participate in forming a triplex helix with a double-stranded nucleic acid in the cell. In certain embodiments, provided ds oligonucleotides may bind to genomic (e.g., chromosomal) nucleic acid. In certain embodiments, provided ds oligonucleotides may bind to genomic (e.g., chromosomal) nucleic acid, thus preventing or decreasing expression of the nucleic acid (e.g., by preventing or decreasing transcription, transcriptional enhancement, modification, etc.). In certain embodiments, provided ds oligonucleotides may bind to DNA quadruplexes. In certain embodiments, provided ds oligonucleotides may be immunomodulatory. In certain embodiments, provided ds oligonucleotides may be immunostimulatory. In certain embodiments, provided oligonucleotides may be immunostimulatory and may comprise a CpG sequence. In certain embodiments, provided ds oligonucleotides may be immunostimulatory and may comprise a CpG sequence and may be useful as an adjuvant. In certain embodiments, provided ds oligonucleotides may be immunostimulatory and may comprise a CpG sequence and may be useful as an adjuvant in treating a disease (e.g., an infectious disease or cancer). In certain embodiments, provided ds oligonucleotides may be therapeutic. In certain embodiments, provided ds oligonucleotides may be non-therapeutic. In certain embodiments, provided ds oligonucleotides may be therapeutic or non-therapeutic. In certain embodiments, provided ds oligonucleotides are useful in therapeutic, diagnostic, research and/or nanomaterials applications. In certain embodiments, provided ds oligonucleotides may be useful for experimental purposes. In certain embodiments, provided ds oligonucleotides may be useful for experimental purposes, e.g., as a probe, in a microarray, etc. In certain embodiments, provided ds oligonucleotides may participate in more than one biological mechanism; in certain such embodiments, for example, provided ds oligonucleotides may participate in both RNAi and RNase H mechanisms.

In certain embodiments, provided ds oligonucleotides are directed to a target (e.g., a target sequence, a target RNA, a target mRNA, a target pre-mRNA, a target gene, etc.). A target gene is a gene with respect to which expression and/or activity of one or more gene products (e.g., RNA and/or protein products) are intended to be altered. In certain embodiments, a target gene is intended to be inhibited. Thus, when a ds oligonucleotide as described herein acts on a particular target gene, presence and/or activity of one or more gene products of that gene are altered when the ds oligonucleotide is present as compared with when it is absent.

In certain embodiments, a target is a specific allele with respect to which expression and/or activity of one or more products (e.g., RNA and/or protein products) are intended to be altered. In certain embodiments, a target allele is one whose presence and/or expression is associated (e.g., correlated) with presence, incidence, and/or severity, of one or more diseases and/or conditions. Alternatively or additionally, in certain embodiments, a target allele is one for which alteration of level and/or activity of one or more gene products correlates with improvement (e.g., delay of onset, reduction of severity, responsiveness to other therapy, etc) in one or more aspects of a disease and/or condition.

In certain embodiments, e.g., where presence and/or activity of a particular allele (a disease-associated allele) is associated (e.g., correlated) with presence, incidence and/or severity of one or more disorders, diseases and/or conditions, a different allele of the same gene exists and is not so associated, or is associated to a lesser extent (e.g., shows less significant, or statistically insignificant correlation), ds oligonucleotides and methods thereof as described herein may preferentially or specifically target the associated allele relative to the one or more less-associated/unassociated allele(s), thus mediating allele-specific suppression.

In certain embodiments, a target sequence is a sequence to which an oligonucleotide as described herein binds. In certain embodiments, a target sequence is identical to, or is an exact complement of, a sequence of a provided oligonucleotide, or of consecutive residues therein (e.g., a provided oligonucleotide includes a target-binding sequence that is identical to, or an exact complement of, a target sequence). In certain embodiments, a target-binding sequence is an exact complement of a target sequence of a transcript (e.g., pre-mRNA, mRNA, etc.). A target-binding sequence/target sequence can be of various lengths to provided oligonucleotides with desired activities and/or properties. In certain embodiments, a target binding sequence/target sequence comprises 5-50 (e.g., 10-40, 15-30, 15-25, 16-25, 17-25, 18-25, 19-25, 20-25, 10, 11, 12, 13, 14, 15, 16, 17, 18, 19, 20, 21, 22, 23, 24, 25, or more) bases. In certain embodiments, a small number of differences/mismatches is tolerated between (a relevant portion of) an oligonucleotide and its target sequence, including but not limited to the 5′ and/or 3′-end regions of the target and/or oligonucleotide sequence. In certain embodiments, a target sequence is present within a target gene. In certain embodiments, a target sequence is present within a transcript (e.g., an mRNA and/or a pre-mRNA) produced from a target gene.

In certain embodiments, a target sequence includes one or more allelic sites (i.e., positions within a target gene at which allelic variation occurs). In certain embodiments, an allelic site is a mutation. In certain embodiments, an allelic site is a SNP. In some such embodiments, a provided oligonucleotide binds to one allele preferentially or specifically relative to one or more other alleles. In certain embodiments, a provided oligonucleotide binds preferentially to a disease-associated allele. For example, in certain embodiments, an oligonucleotide (or a target-binding sequence portion thereof) provided herein has a sequence that is, fully or at least in part, identical to, or an exact complement of a particular allelic version of a target sequence.

In certain embodiments, an oligonucleotide (or a target-binding sequence portion thereof) provided herein has a sequence that is identical to, or an exact complement of a target sequence comprising an allelic site, or an allelic site, of a disease-associated allele. In certain embodiments, an oligonucleotide provided herein has a target binding sequence that is an exact complement of a target sequence comprising an allelic site of a transcript of an allele (in certain embodiments, a disease-associated allele), wherein the allelic site is a mutation. In certain embodiments, an oligonucleotide provided herein has a target binding sequence that is an exact complement of a target sequence comprising an allelic site of a transcript of an allele (in certain embodiments, a disease-associated allele), wherein the allelic site is a SNP. In certain embodiments, a sequence is any sequence disclosed herein.

Unless otherwise noted, all sequences (including, but not limited to base sequences and patterns of chemistry, modification, and/or stereochemistry) are presented in 5′ to 3′ order, with the 5′ terminal nucleotide identified as the “+1” position and the 3′ terminal nucleotide identified either by the number of nucleotides of the full sequence or by “N”, with the penultimate nucleotide identified, e.g., as “N−1”, and so on.

In certain embodiments, the present disclosure provides compositions and methods related to an oligonucleotide which is specific to a target and which has any format, structural element or base sequence of any oligonucleotide disclosed herein.

In certain embodiments, the present disclosure provides compositions and methods related to an oligonucleotide which is specific to a target and which has or comprises the base sequence of any oligonucleotide disclosed herein, or a region of at least 15 contiguous nucleotides of the base sequence of any oligonucleotide disclosed herein, wherein the first nucleotide of the base sequence or the first nucleotide of the at least 15 contiguous nucleotides can be optionally replaced by T or DNA T.

In certain embodiments, the present disclosure provides compositions and methods for RNA interference directed by a RNAi agent (also referred to as a RNAi oligonucleotides). In certain embodiments, oligonucleotides of such compositions can have a format, structural element or base sequence of an oligonucleotide disclosed herein.

In certain embodiments, the present disclosure provides compositions and methods for RNase H-mediated knockdown of a target gene RNA directed by an oligonucleotide (e.g., an antisense oligonucleotide).

Provided oligonucleotides and oligonucleotide compositions can have any format, structural element or base sequence of any oligonucleotide disclosed herein. In certain embodiments, a structural element is a 5′-end structure, 5′-end region, 5′-nucleotide, seed region, post-seed region, 3′-end region, 3′-terminal dinucleotide, 3′-end cap, or any portion of any of these structures, GC content, long GC stretch, and/or any modification, chemistry, stereochemistry, pattern of modification, chemistry or stereochemistry, or a chemical moiety (e.g., including but not limited to, a targeting moiety, a lipid moiety, a GalNAc moiety, a carbohydrate moiety, etc.), any component, or any combination of any of the above.

In certain embodiments, the present disclosure provides compositions and methods of use of an oligonucleotide.

In certain embodiments, the present disclosure provides compositions and methods of use of an oligonucleotide which can direct both RNA interference and RNase H-mediated knockdown of a target gene RNA. In certain embodiments, oligonucleotides of such compositions can have a format, structural element or base sequence of an oligonucleotide disclosed herein.

In certain embodiments, an oligonucleotide directing a particular event or activity participates in the particular event or activity, e.g., a decrease in the expression, level or activity of a target gene or a gene product thereof. In certain embodiments, an oligonucleotide is deemed to “direct” a particular event or activity when presence of the oligonucleotide in a system in which the event or activity can occur correlates with increased detectable incidence, frequency, intensity and/or level of the event or activity.

In certain embodiments, a provided oligonucleotide comprises any one or more structural elements of an oligonucleotide as described herein, e.g., a base sequence (or a portion thereof of at least 15 contiguous bases); a pattern of internucleotidic linkages (or a portion thereof of at least 5 contiguous internucleotidic linkage); a pattern of stereochemistry of internucleotidic linkages (or a portion thereof of at least 5 contiguous internucleotidic linkages); a 5′-end structure; a 5′-end region; a first region; a second region; and a 3′-end region (which can be a 3′-terminal dinucleotide and/or a 3′-end cap); and an optional additional chemical moiety; and, in certain embodiments, at least one structural element comprises a chirally controlled chiral center. In certain embodiments, a 3′-terminal dinucleotide can comprise two total nucleotides. In certain embodiments, an oligonucleotide further comprises a chemical moiety selected from, as non-limiting examples, a targeting moiety, a carbohydrate moiety, a GalNAc moiety, a lipid moiety, and any other chemical moiety described herein or known in the art. In certain embodiments, a moiety that binds APGR is a moiety of GalNAc, or a variant, derivative or modified version thereof, as described herein and/or known in the art. In certain embodiments, an oligonucleotide is a RNAi agent. In certain embodiments, a first region is a seed region. In certain embodiments, a second region is a post-seed region.

In certain embodiments, a provided oligonucleotide comprises any one or more structural elements of a RNAi agent as described herein, e.g., a 5′-end structure; a 5′-end region; a seed region; a post-seed region (the region between the seed region and the 3′-end region); and a 3′-end region (which can be a 3′-terminal dinucleotide and/or a 3′-end cap); and an optional additional chemical moiety; and, in certain embodiments, at least one structural element comprises a chirally controlled chiral center. In certain embodiments, a 3′-terminal dinucleotide can comprise two total nucleotides. In certain embodiments, an oligonucleotide further comprises a chemical moiety selected from, as non-limiting examples, a targeting moiety, a carbohydrate moiety, a GalNAc moiety, and a lipid moiety. In certain embodiments, a moiety that binds APGR is any GalNAc, or variant, derivative or modification thereof, as described herein or known in the art.

In certain embodiments, a provided oligonucleotide comprises any one or more structural elements of an oligonucleotide as described herein, e.g., a 5′-end structure, a 5′-end region, a first region, a second region, a 3′-end region, and an optional additional chemical moiety, wherein at least one structural element comprises a chirally controlled chiral center. In certain embodiments, the oligonucleotide comprises a span of at least 5 total nucleotides without 2′-modifications. In certain embodiments, the oligonucleotide further comprises an additional chemical moiety selected from, as non-limiting examples, a targeting moiety, a carbohydrate moiety, a GalNAc moiety, and a lipid moiety. In certain embodiments, a provided oligonucleotide is capable of directing RNA interference. In certain embodiments, a provided oligonucleotide is capable of directing RNase H-mediated knockdown. In certain embodiments, a provided oligonucleotide is capable of directing both RNA interference and RNase H-mediated knockdown. In certain embodiments, a first region is a seed region. In certain embodiments, a second region is a post-seed region.

In certain embodiments, a provided oligonucleotide comprises any one or more structural elements of a RNAi agent, e.g., a 5′-end structure, a 5′-end region, a seed region, a post-seed region, and a 3′-end region and an optional additional chemical moiety, wherein at least one structural element comprises a chirally controlled chiral center; and, in certain embodiments, the oligonucleotide is also capable of directing RNase H-mediated knockdown of a target gene RNA. In certain embodiments, the oligonucleotide comprises a span of at least 5 total 2′-deoxy nucleotides. In certain embodiments, the oligonucleotide further comprises a chemical moiety selected from, as non-limiting examples, a targeting moiety, a carbohydrate moiety, a GalNAc moiety, and a lipid moiety, and any other additional chemical moiety described herein.

In certain embodiments, the present disclosure demonstrates that oligonucleotide properties can be modulated through chemical modifications. In certain embodiments, the present disclosure provides an oligonucleotide composition comprising a first plurality of oligonucleotides which have a common base sequence and comprise one or more internucleotidic linkage, sugar, and/or base modifications. In certain embodiments, the present disclosure provides an oligonucleotide composition capable of directing RNA interference and comprising a first plurality of oligonucleotides which have a common base sequence and comprise one or more internucleotidic linkage, and/or one or more sugar, and/or one or more base modifications. In certain embodiments, an oligonucleotide or oligonucleotide composition is also capable of directing RNase H-mediated knockdown of a target gene RNA. In certain embodiments, the present disclosure demonstrates that oligonucleotide properties, e.g., activities, toxicities, etc., can be modulated through chemical modifications of sugars, nucleobases, and/or internucleotidic linkages. In certain embodiments, the present disclosure provides an oligonucleotide composition comprising a plurality of oligonucleotides which have a common base sequence, and comprise one or more modified internucleotidic linkages (or “non-natural internucleotidic linkages”, linkages that can be utilized in place of a natural phosphate internucleotidic linkage (—OP(O)(OH)O—, which may exist as a salt form (—OP(O)(O⁻)O—) at a physiological pH) found in natural DNA and RNA), one or more modified sugar moieties, and/or one or more natural phosphate linkages. In certain embodiments, provided oligonucleotides may comprise two or more types of modified internucleotidic linkages. In certain embodiments, a provided oligonucleotide comprises a non-negatively charged internucleotidic linkage. In certain embodiments, a non-negatively charged internucleotidic linkage is a neutral internucleotidic linkage. In certain embodiments, a neutral internucleotidic linkage comprises a cyclic guanidine moiety. Such moieties an optionally substituted. In certain embodiments, a provided oligonucleotide comprises a neutral internucleotidic linkage and another internucleotidic linkage which is not a neutral backbone. In certain embodiments, a provided oligonucleotide comprises a neutral internucleotidic linkage and a phosphorothioate internucleotidic linkage. In certain embodiments, provided oligonucleotide compositions comprising a plurality of oligonucleotides are chirally controlled and level of the plurality of oligonucleotides in the composition is controlled or pre-determined, and oligonucleotides of the plurality share a common stereochemistry configuration at one or more chiral internucleotidic linkages. For example, in certain embodiments, oligonucleotides of a plurality share a common stereochemistry configuration at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 or more chiral internucleotidic linkages, each of which is independently Rp or Sp; in certain embodiments, oligonucleotides of a plurality share a common stereochemistry configuration at each chiral internucleotidic linkages. In certain embodiments, a chiral internucleotidic linkage where a controlled level of oligonucleotides of a composition share a common stereochemistry configuration (independently in the Rp or Sp configuration) is referred to as a chirally controlled internucleotidic linkage. In certain embodiments, a modified internucleotidic linkage is a non-negatively charged (neutral or cationic) internucleotidic linkage in that at a pH, (e.g., human physiological pH (˜7.4), pH of a delivery site (e.g., an organelle, cell, tissue, organ, organism, etc.), etc.), it largely (e.g., at least 5%, 10%, 20%, 30%, 40%, 50%, 60%, 70%, 80%, 90%, etc.; in certain embodiments, at least 30%; in certain embodiments, at least 40%; in certain embodiments, at least 50%; in certain embodiments, at least 60%; in certain embodiments, at least 70%; in certain embodiments, at least 80%; in certain embodiments, at least 90%; in certain embodiments, at least 99%; etc.;) exists as a neutral or cationic form (as compared to an anionic form (e.g., —O—P(O)(O⁻)—O— (the anionic form of natural phosphate linkage), —O—P(O)(S⁻)—O— (the anionic form of phosphorothioate linkage), etc.)), respectively. In certain embodiments, a modified internucleotidic linkage is a neutral internucleotidic linkage in that at a pH, it largely exists as a neutral form. In certain embodiments, a modified internucleotidic linkage is a cationic internucleotidic linkage in that at a pH, it largely exists as a cationic form. In certain embodiments, a pH is human physiological pH (˜7.4). In certain embodiments, a modified internucleotidic linkage is a neutral internucleotidic linkage in that at pH 7.4 in a water solution, at least 90% of the internucleotidic linkage exists as its neutral form. In certain embodiments, a modified internucleotidic linkage is a neutral internucleotidic linkage in that in a water solution of the oligonucleotide, at least 50%, 60%, 70%, 80%, 90%, 95%, or 99% of the internucleotidic linkage exists in its neutral form. In certain embodiments, the percentage is at least 90%. In certain embodiments, the percentage is at least 95%. In certain embodiments, the percentage is at least 99%. In certain embodiments, a non-negatively charged internucleotidic linkage, e.g., a neutral internucleotidic linkage, when in its neutral form has no moiety with a pKa that is less than 8, 9, 10, 11, 12, 13, or 14. In certain embodiments, pKa of an internucleotidic linkage in the present disclosure can be represented by pKa of CH₃— the internucleotidic linkage —CH₃ (i.e., replacing the two nucleoside units connected by the internucleotidic linkage with two —CH₃ groups). Without wishing to be bound by any particular theory, in at least some cases, a neutral internucleotidic linkage in an oligonucleotide can provide improved properties and/or activities, e.g., improved delivery, improved resistance to exonucleases and endonucleases, improved cellular uptake, improved endosomal escape and/or improved nuclear uptake, etc., compared to a comparable nucleic acid which does not comprises a neutral internucleotidic linkage.

In certain embodiments, a non-negatively charged internucleotidic linkage has the structure of e.g., of formula I-n-1, I-n-2, I-n-3, II, II-a-1, II-a-2, II-b-1, II-b-2, II-c-1, II-c-2, II-d-1, II-d-2, as described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612 etc. In certain embodiments, a non-negatively charged internucleotidic linkage comprises a cyclic guanidine moiety. In certain embodiments, a modified internucleotidic linkage comprising a cyclic guanidine moiety has the structure of:

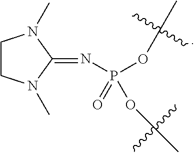 [SMILES: *OP(=O)(N=C1N(C)CCN1C)O*]

In certain embodiments, a neutral internucleotidic linkage comprising a cyclic guanidine moiety is chirally controlled. In certain embodiments, the present disclosure pertains to a composition comprising an oligonucleotide comprising at least one neutral internucleotidic linkage and at least one phosphorothioate internucleotidic linkage.

In certain embodiments, the present disclosure pertains to a composition comprising an oligonucleotide comprising at least one neutral internucleotidic linkage and at least one phosphorothioate internucleotidic linkage, wherein the phosphorothioate internucleotidic linkage is a chirally controlled internucleotidic linkage in the Sp configuration.

In certain embodiments, the present disclosure pertains to a composition comprising an oligonucleotide comprising at least one neutral internucleotidic linkage and at least one phosphorothioate internucleotidic linkage, wherein the phosphorothioate is a chirally controlled internucleotidic linkage in the Rp configuration.

In certain embodiments, the present disclosure pertains to a composition comprising an oligonucleotide comprising at least one neutral internucleotidic linkage of a neutral internucleotidic linkage comprising a Tmg group

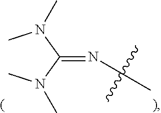 [SMILES: *N=C(N(C)C)N(C)C]

and at least one phosphorothioate.

In certain embodiments, each internucleotidic linkage in an oligonucleotide is independently selected from a natural phosphate linkage, a phosphorothioate linkage, and anon-negatively charged internucleotidic linkage (e.g., n001, n003, n004, n006, n008, n009, n013, n020, n021, n025, n026, n029, n031, n033, n037, n043, n046, n047, n048, n054, n058, or n055). In some embodiments, each internucleotidic linkage in an oligonucleotide is independently selected from a natural phosphate linkage, a phosphorothioate linkage, and a neutral internucleotidic linkage (e.g., n001, n003, n004, n006, n008, n009, n013, n020, n021, n025, n026, n029, n031, n033, n037, n043, n046, n047, n048, n054, n058, or n055).

In certain embodiments, the present disclosure pertains to a composition comprising an oligonucleotide comprising at least one neutral internucleotidic linkage of a neutral internucleotidic linkage comprising a Tmg group, and at least one phosphorothioate, wherein the phosphorothioate is a chirally controlled internucleotidic linkage in the Sp configuration.

In certain embodiments, the present disclosure pertains to a composition comprising an oligonucleotide comprising at least one neutral internucleotidic linkage selected from a neutral internucleotidic linkage of a neutral internucleotidic linkage comprising a Tmg group, and at least one phosphorothioate, wherein the phosphorothioate is a chirally controlled internucleotidic linkage in the Rp configuration.

Various types of internucleotidic linkages differ in properties. Without wishing to be bound by any theory, the present disclosure notes that a natural phosphate linkage (phosphodiester internucleotidic linkage) is anionic and may be unstable when used by itself without other chemical modifications in vivo; a phosphorothioate internucleotidic linkage is anionic, generally more stable in vivo than a natural phosphate linkage, and generally more hydrophobic; a neutral internucleotidic linkage such as one exemplified in the present disclosure comprising a cyclic guanidine moiety is neutral at physiological pH, can be more stable in vivo than a natural phosphate linkage, and more hydrophobic.

In certain embodiments, a chirally controlled neutral internucleotidic linkage sis neutral at physiological pH, chirally controlled, stable in vivo, hydrophobic, and may increase endosomal escape.

In certain embodiments, provided oligonucleotides comprise one or more regions, e.g., a block, wing, core, 5′-end, 3′-end, middle, seed, post-seed region, etc. In certain embodiments, a region (e.g., a block, wing, core, 5′-end, 3′-end, middle region, etc.) comprises a non-negatively charged internucleotidic linkage, e.g., of formula I-n-1, I-n-2, I-n-3, II, II-a-1, II-a-2, II-b-1, II-b-2, II-c-1, II-c-2, II-d-1, II-d-2, etc as described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612. In certain embodiments, a region comprises a neutral internucleotidic linkage. In certain embodiments, a region comprises an internucleotidic linkage which comprises a cyclic guanidine guanidine. In certain embodiments, a region comprises an internucleotidic linkage which comprises a cyclic guanidine moiety. In certain embodiments, a region comprises an internucleotidic linkage having the structure of

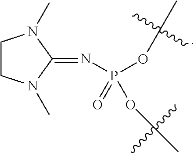 [SMILES: *OP(=O)(N=C1N(C)CCN1C)O*]

In certain embodiments, such internucleotidic linkages are chirally controlled.

In certain embodiments, a nucleotide is a natural nucleotide. In certain embodiments, a nucleotide is a modified nucleotide. In certain embodiments, a nucleotide is a nucleotide analog. In certain embodiments, a base is a modified base. In certain embodiments, a base is protected nucleobase, such as a protected nucleobase used in oligonucleotide synthesis. In certain embodiments, a base is a base analog. In certain embodiments, a sugar is a modified sugar. In certain embodiments, a sugar is a sugar analog. In certain embodiments, an internucleotidic linkage is a modified internucleotidic linkage. In certain embodiments, a nucleotide comprises a base, a sugar, and an internucleotidic linkage, wherein each of the base, the sugar, and the internucleotidic linkage is independently and optionally naturally-occurring or non-naturally occurring. In certain embodiments, a nucleoside comprises a base and a sugar, wherein each of the base and the sugar is independently and optionally naturally-occurring or non-naturally occurring. Non-limiting examples of nucleotides include DNA (2′-deoxy) and RNA (2′-OH) nucleotides; and those which comprise one or more modifications at the base, sugar and/or internucleotidic linkage. Non-limiting examples of sugars include ribose and deoxyribose; and ribose and deoxyribose with 2′-modifications, including but not limited to 2′-F, LNA, 2′-OMe, and 2′-MOE modifications. In certain embodiments, an internucleotidic linkage is a moiety which does not a comprise a phosphorus but serves to link two natural or non-natural sugars.

In certain embodiments, a composition comprises a multimer of two or more of any: oligonucleotides of a first plurality and/or oligonucleotides of a second plurality, wherein the oligonucleotides of the first and second plurality can independently direct knockdown of the same or different targets independently via RNA interference and/or RNase H-mediated knockdown.

In certain embodiments, the present disclosure provides an oligonucleotide composition comprising a first plurality of oligonucleotides which share:
1) a common base sequence;
2) a common pattern of backbone linkages;
3) common stereochemistry independently at at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 chiral internucleotidic linkages (“chirally controlled internucleotidic linkages”); which composition is chirally controlled in that level of the first plurality of oligonucleotides in the composition is predetermined.

In certain embodiments, an oligonucleotide composition comprising a plurality of oligonucleotides (e.g., a first plurality of oligonucleotides) is chirally controlled in that oligonucleotides of the plurality share a common stereochemistry independently at one or more chiral internucleotidic linkages. In certain embodiments, oligonucleotides of the plurality share a common stereochemistry configuration at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 or more chiral internucleotidic linkages, each of which is independently Rp or Sp In certain embodiments, oligonucleotides of the plurality share a common stereochemistry configuration at each chiral internucleotidic linkages. In certain embodiments, a chiral internucleotidic linkage where a predetermined level of oligonucleotides of a composition share a common stereochemistry configuration (independently Rp or Sp) is referred to as a chirally controlled internucleotidic linkage.

In certain embodiments, a predetermined level of oligonucleotides of a provided composition, e.g., a first plurality of oligonucleotides of certain example compositions, comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 or more chirally controlled internucleotidic linkages.

In certain embodiments, at least 5 internucleotidic linkages are chirally controlled; in certain embodiments, at least 10 internucleotidic linkages are chirally controlled; in certain embodiments, at least 15 internucleotidic linkages are chirally controlled; in certain embodiments, each chiral internucleotidic linkage is chirally controlled.

In certain embodiments, 1%-100% of chiral internucleotidic linkages are chirally controlled. In certain embodiments, at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of chiral internucleotidic linkages are chirally controlled.

In certain embodiments, the present disclosure provides an oligonucleotide composition comprising a first plurality of oligonucleotides which share:
1) a common base sequence;
2) a common pattern of backbone linkages; and
3) a common pattern of backbone chiral centers, which composition is a substantially pure preparation of oligonucleotide in that a predetermined level of the oligonucleotides in the composition have the common base sequence and length, the common pattern of backbone linkages, and the common pattern of backbone chiral centers. In certain embodiments, the common pattern of backbone chiral centers comprises at least one internucleotidic linkage comprising a chirally controlled chiral center. In certain embodiments, a predetermined level of oligonucleotides is at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in a provided composition. In certain embodiments, a predetermined level of oligonucleotides is at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in a provided composition that are of or comprise a common base sequence. In certain embodiments, all oligonucleotides in a provided composition that are of or comprise a common base sequence are at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in the composition. In certain embodiments, a predetermined level of oligonucleotides is at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in a provided composition that are of or comprise a common base sequence, base modification, sugar modification and/or modified internucleotidic linkage. In certain embodiments, all oligonucleotides in a provided composition that are of or comprise a common base sequence, base modification, sugar modification and/or modified internucleotidic linkage are at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in the composition. In certain embodiments, a predetermined level of oligonucleotides is at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in a provided composition that are of or comprise a common base sequence, pattern of base modification, pattern of sugar modification, and/or pattern of modified internucleotidic linkage. In certain embodiments, all oligonucleotides in a provided composition that are of or comprise a common base sequence, pattern of base modification, pattern of sugar modification, and/or pattern of modified internucleotidic linkage are at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in the composition. In certain embodiments, a predetermined level of oligonucleotides is at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in a provided composition that share a common base sequence, a common pattern of base modification, a common pattern of sugar modification, and/or a common pattern of modified internucleotidic linkages. In certain embodiments, all oligonucleotides in a provided composition that share a common base sequence, a common pattern of base modification, a common pattern of sugar modification, and/or a common pattern of modified internucleotidic linkages are at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in the composition. In certain embodiments, a predetermined level is 1-100%. In certain embodiments, a predetermined level is at least 1%. In certain embodiments, a predetermined level is at least 5%. In certain embodiments, a predetermined level is at least 10%. In certain embodiments, a predetermined level is at least 20%. In certain embodiments, a predetermined level is at least 30%. In certain embodiments, a predetermined level is at least 40%. In certain embodiments, a predetermined level is at least 50%. In certain embodiments, a predetermined level is at least 60%. In certain embodiments, a predetermined level is at least 10%. In certain embodiments, a predetermined level is at least 70%. In certain embodiments, a predetermined level is at least 80%. In certain embodiments, a predetermined level is at least 90%. In certain embodiments, a predetermined level is at least 5*(½g), wherein g is the number of chirally controlled internucleotidic linkages. In certain embodiments, a predetermined level is at least 10*(½g), wherein g is the number of chirally controlled internucleotidic linkages. In certain embodiments, a predetermined level is at least 100*(½g), wherein g is the number of chirally controlled internucleotidic linkages. In certain embodiments, a predetermined level is at least (0.80)g, wherein g is the number of chirally controlled internucleotidic linkages. In certain embodiments, a predetermined level is at least (0.80)g, wherein g is the number of chirally controlled internucleotidic linkages. In certain embodiments, a predetermined level is at least (0.80)g, wherein g is the number of chirally controlled internucleotidic linkages. In certain embodiments, a predetermined level is at least (0.85)g, wherein g is the number of chirally controlled internucleotidic linkages. In certain embodiments, a predetermined level is at least (0.90)g, wherein g is the number of chirally controlled internucleotidic linkages. In certain embodiments, a predetermined level is at least (0.95)g, wherein g is the number of chirally controlled internucleotidic linkages. In certain embodiments, a predetermined level is at least (0.96)g, wherein g is the number of chirally controlled internucleotidic linkages. In certain embodiments, a predetermined level is at least (0.97)g, wherein g is the number of chirally controlled internucleotidic linkages. In certain embodiments, a predetermined level is at least (0.98)g, wherein g is the number of chirally controlled internucleotidic linkages. In certain embodiments, a predetermined level is at least (0.99)g, wherein g is the number of chirally controlled internucleotidic linkages. In certain embodiments, to determine level of oligonucleotides having g chirally controlled internucleotidic linkages in a composition, product of diastereopurity of each of the g chirally controlled internucleotidic linkages: (diastereopurity of chirally controlled internucleotidic linkage 1)*(diastereopurity of chirally controlled internucleotidic linkage 2) * . . . * (diastereopurity of chirally controlled internucleotidic linkage g) is utilized as the level, wherein diastereopurity of each chirally controlled internucleotidic linkage is independently represented by diastereopurity of a dimer comprising the same internucleotidic linkage and nucleosides flanking the internucleotidic linkage and prepared under comparable methods as the oligonucleotides (e.g., comparable or preferably identical oligonucleotide preparation cycles, including comparable or preferably identical reagents and reaction conditions). In certain embodiments, levels of oligonucleotides and/or diastereopurity can be determined by analytical methods, e.g., chromatographic, spectrometric, spectroscopic methods or any combinations thereof. Among other things, the present disclosure encompasses the recognition that stereorandom oligonucleotide preparations contain a plurality of distinct chemical entities that differ from one another, e.g., in the stereochemical structure (or stereochemistry) of individual backbone chiral centers within the oligonucleotide chain. Without control of stereochemistry of backbone chiral centers, stereorandom oligonucleotide preparations provide uncontrolled compositions comprising undetermined levels of oligonucleotide stereoisomers. Even though these stereoisomers may have the same base sequence and/or chemical modifications, they are different chemical entities at least due to their different backbone stereochemistry, and they can have, as demonstrated herein, different properties, e.g., sensitivity to nucleases, activities, distribution, etc. In certain embodiments, a particular stereoisomer may be defined, for example, by its base sequence, its length, its pattern of backbone linkages, and its pattern of backbone chiral centers. In certain embodiments, the present disclosure demonstrates that improvements in properties and activities achieved through control of stereochemistry within an oligonucleotide can be comparable to, or even better than those achieved through use of chemical modification.

Among other things, the present disclosure encompasses the recognition that stereorandom oligonucleotide preparations contain a plurality of distinct chemical entities that differ from one another, e.g., in the stereochemical structure (or stereochemistry) of individual backbone chiral centers within the oligonucleotide chain. Without control of stereochemistry of backbone chiral centers, stereorandom oligonucleotide preparations provide uncontrolled compositions comprising undetermined levels of oligonucleotide stereoisomers. Even though these stereoisomers may have the same base sequence and/or chemical modifications, they are different chemical entities at least due to their different backbone stereochemistry, and they can have, as demonstrated herein, different properties, e.g., sensitivity to nucleases, activities, distribution, etc. In certain embodiments, a particular stereoisomer may be defined, for example, by its base sequence, its length, its pattern of backbone linkages, and its pattern of backbone chiral centers. In certain embodiments, the present disclosure demonstrates that improvements in properties and activities achieved through control of stereochemistry within an oligonucleotide can be comparable to, or even better than those achieved through use of chemical modification.

### I. DETAILED DESCRIPTION OF CERTAIN EMBODIMENTS

Technologies of the present disclosure may be understood more readily by reference to the following detailed description of certain embodiments.

### Definitions

As used herein, the following definitions shall apply unless otherwise indicated. For purposes of this disclosure, the elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001.

As used herein in the present disclosure, unless otherwise clear from context, (i) the term “a” or “an” may be understood to mean “at least one”; (ii) the term “or” may be understood to mean “and/or”; (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more; (v) the terms “about” and “approximately” may be understood to permit standard variation as would be understood by those of ordinary skill in the art; and (vi) where ranges are provided, endpoints are included.

Unless otherwise specified, description of oligonucleotides and elements thereof (e.g., base sequence, sugar modifications, internucleotidic linkages, linkage phosphorus stereochemistry, patterns thereof, etc.) is from 5′ to 3′, with the 5′ terminal nucleotide identified as the “+1” position and the 3′ terminal nucleotide identified either by the number of nucleotides of the full sequence or by “N”, with the penultimate nucleotide identified, e.g., as “N−1”, and so on. As those skilled in the art will appreciate, in certain embodiments, oligonucleotides may be provided and/or utilized as salt forms, particularly pharmaceutically acceptable salt forms, e.g., sodium salts. As those skilled in the art will also appreciate, in certain embodiments, individual oligonucleotides within a composition may be considered to be of the same constitution and/or structure even though, within such composition (e.g., a liquid composition), particular such oligonucleotides might be in different salt form(s) (and may be dissolved and the oligonucleotide chain may exist as an anion form when, e.g., in a liquid composition) at a particular moment in time. For example, those skilled in the art will appreciate that, at a given pH, individual internucleotidic linkages along an oligonucleotide chain may be in an acid (H) form, or in one of a plurality of possible salt forms (e.g., a sodium salt, or a salt of a different cation, depending on which ions might be present in the preparation or composition), and will understand that, so long as their acid forms (e.g., replacing all cations, if any, with H⁺) are of the same constitution and/or structure, such individual oligonucleotides may properly be considered to be of the same constitution and/or structure.

Aliphatic: As used herein, “aliphatic” means a straight-chain (i.e., unbranched) or branched, substituted or unsubstituted hydrocarbon chain that is completely saturated or that contains one or more units of unsaturation (but not aromatic), or a substituted or unsubstituted monocyclic, bicyclic, or polycyclic hydrocarbon ring that is completely saturated or that contains one or more units of unsaturation (but not aromatic), or combinations thereof. In certain embodiments, aliphatic groups contain 1-50 aliphatic carbon atoms. In certain embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Alkenyl: As used herein, the term “alkenyl” refers to an aliphatic group, as defined herein, having one or more double bonds.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In certain embodiments, cycloalkyl rings have from about 3-10 carbon atoms in their ring structure where such rings are monocyclic, bicyclic, or polycyclic, and alternatively about 5, 6 or 7 carbons in the ring structure. In certain embodiments, an alkyl group may be a lower alkyl group, wherein a lower alkyl group comprises 1-4 carbon atoms (e.g., C₁-C₄ for straight chain lower alkyls).

Alkynyl: As used herein, the term “alkynyl” refers to an aliphatic group, as defined herein, having one or more triple bonds.

Analog: The term “analog” includes any chemical moiety which differs structurally from a reference chemical moiety or class of moieties, but which is capable of performing at least one function of such a reference chemical moiety or class of moieties. As non-limiting examples, a nucleotide analog differs structurally from a nucleotide but performs at least one function of a nucleotide; a nucleobase analog differs structurally from a nucleobase but performs at least one function of a nucleobase; etc.

Animal: As used herein, the term “animal” refers to any member of the animal kingdom. In certain embodiments, “animal” refers to humans, at any stage of development. In certain embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate and/or a pig). In certain embodiments, animals include, but are not limited to, mammals, birds, reptiles, amphibians, fish and/or worms. In certain embodiments, an animal may be a transgenic animal, a genetically-engineered animal and/or a clone.

Aryl: The term “aryl”, as used herein, used alone or as part of a larger moiety as in “aralkyl,” “aralkoxy,” or “aryloxyalkyl,” refers to monocyclic, bicyclic or polycyclic ring systems having a total of five to thirty ring members, wherein at least one ring in the system is aromatic. In certain embodiments, an aryl group is a monocyclic, bicyclic or polycyclic ring system having a total of five to fourteen ring members, wherein at least one ring in the system is aromatic, and wherein each ring in the system contains 3 to 7 ring members. In certain embodiments, each monocyclic ring unit is aromatic. In certain embodiments, an aryl group is a biaryl group. The term “aryl” may be used interchangeably with the term “aryl ring.” In certain embodiments of the present disclosure, “aryl” refers to an aromatic ring system which includes, but is not limited to, phenyl, biphenyl, naphthyl, binaphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

Chiral control: As used herein, “chiral control” refers to control of the stereochemical designation of the chiral linkage phosphorus in a chiral internucleotidic linkage within an oligonucleotide. As used herein, a chiral internucleotidic linkage is an internucleotidic linkage whose linkage phosphorus is chiral. In certain embodiments, a control is achieved through a chiral element that is absent from the sugar and base moieties of an oligonucleotide, for example, in certain embodiments, a control is achieved through use of one or more chiral auxiliaries during oligonucleotide preparation, which chiral auxiliaries often are part of chiral phosphoramidites used during oligonucleotide preparation. In contrast to chiral control, a person having ordinary skill in the art will appreciate that conventional oligonucleotide synthesis which does not use chiral auxiliaries cannot control stereochemistry at a chiral internucleotidic linkage if such conventional oligonucleotide synthesis is used to form the chiral internucleotidic linkage. In certain embodiments, the stereochemical designation of each chiral linkage phosphorus in each chiral internucleotidic linkage within an oligonucleotide is controlled.

Chirally controlled oligonucleotide composition: The terms “chirally controlled oligonucleotide composition”, “chirally controlled nucleic acid composition”, and the like, as used herein, refers to a composition that comprises a plurality of oligonucleotides (or nucleic acids) which share a common base sequence, wherein the plurality of oligonucleotides (or nucleic acids) share the same linkage phosphorus stereochemistry at one or more chiral internucleotidic linkages (chirally controlled or stereodefined internucleotidic linkages, whose chiral linkage phosphorus is Rp or Sp in the composition (“stereodefined”), not a random Rp and Sp mixture as non-chirally controlled internucleotidic linkages). In certain embodiments, a chirally controlled oligonucleotide composition comprises a plurality of oligonucleotides (or nucleic acids) that share: 1) a common base sequence, 2) a common pattern of backbone linkages, and 3) a common pattern of backbone phosphorus modifications, wherein the plurality of oligonucleotides (or nucleic acids) share the same linkage phosphorus stereochemistry at one or more chiral internucleotidic linkages (chirally controlled or stereodefined internucleotidic linkages, whose chiral linkage phosphorus is Rp or Sp in the composition (“stereodefined”), not a random Rp and Sp mixture as non-chirally controlled internucleotidic linkages). Level of the plurality of oligonucleotides (or nucleic acids) in a chirally controlled oligonucleotide composition is pre-determined/controlled or enriched (e.g., through chirally controlled oligonucleotide preparation to stereoselectively form one or more chiral internucleotidic linkages) compared to a random level in a non-chirally controlled oligonucleotide composition. In certain embodiments, about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition are oligonucleotides of the plurality. In certain embodiments, about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition that share the common base sequence, the common pattern of backbone linkages, and the common pattern of backbone phosphorus modifications are oligonucleotides of the plurality. In certain embodiments, a level is about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a composition, or of all oligonucleotides in a composition that share a common base sequence (e.g., of a plurality of oligonucleotide or an oligonucleotide type), or of all oligonucleotides in a composition that share a common base sequence, a common pattern of backbone linkages, and a common pattern of backbone phosphorus modifications, or of all oligonucleotides in a composition that share a common base sequence, a common patter of base modifications, a common pattern of sugar modifications, a common pattern of internucleotidic linkage types, and/or a common pattern of internucleotidic linkage modifications. In certain embodiments, the plurality of oligonucleotides share the same stereochemistry at about 1-50 (e.g., about 1-10, 1-20, 5-10, 5-20, 10-15, 10-20, 10-25, 10-30, or about 1, 2, 3, 4, 5, 6, 7, 8, 9, 10, 11, 12, 13, 14, 15, 16, 17, 18, 19, or 20, or at least 1, 2, 3, 4, 5, 6, 7, 8, 9, 10, 11, 12, 13, 14, 15, 16, 17, 18, 19, or 20) chiral internucleotidic linkages. In certain embodiments, the plurality of oligonucleotides share the same stereochemistry at about 1%-100% (e.g., about 5%-100%, 10%-100%, 20%-100%, 30%-100%, 40%-100%, 50%-100%, 60%-100%, 70%-100%, 80-100%, 90-100%, 95-100%, 50%-90%, about 5%, 10%, 15%, 20%, 25%, 30%, 35%, 40%, 45%, 50%, 55%, 60%, 65%, 70%, 75%, 80%, 85%, 90%, 95%, or 100%, or at least 5%, 10%, 15%, 20%, 25%, 30%, 35%, 40%, 45%, 50%, 55%, 60%, 65%, 70%, 75%, 80%, 85%, 90%, 95%, or 99%) of chiral internucleotidic linkages. In certain embodiments, oligonucleotides (or nucleic acids) of a plurality share the same pattern of sugar and/or nucleobase modifications, in any. In certain embodiments, oligonucleotides (or nucleic acids) of a plurality are various forms of the same oligonucleotide (e.g., acid and/or various salts of the same oligonucleotide). In certain embodiments, oligonucleotides (or nucleic acids) of a plurality are of the same constitution. In certain embodiments, level of the oligonucleotides (or nucleic acids) of the plurality is about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides (or nucleic acids) in a composition that share the same constitution as the oligonucleotides (or nucleic acids) of the plurality. In certain embodiments, each chiral internucleotidic linkage is a chiral controlled internucleotidic linkage, and the composition is a completely chirally controlled oligonucleotide composition. In certain embodiments, oligonucleotides (or nucleic acids) of a plurality are structurally identical. In certain embodiments, a chirally controlled internucleotidic linkage has a diastereopurity of at least 80%, 85%, 90%, 91%, 92%, 93%, 94%, 95%, 96%, 97%, 98%, 99% or 99.5%, typically at least 90%, 91%, 92%, 93%, 94%, 95%, 96%, 97%, 98%, 99% or 99.5%. In certain embodiments, a chirally controlled internucleotidic linkage has a diastereopurity of at least 95%. In certain embodiments, a chirally controlled internucleotidic linkage has a diastereopurity of at least 96%. In certain embodiments, a chirally controlled internucleotidic linkage has a diastereopurity of at least 97%. In certain embodiments, a chirally controlled internucleotidic linkage has a diastereopurity of at least 98%. In certain embodiments, a chirally controlled internucleotidic linkage has a diastereopurity of at least 99%. In certain embodiments, a percentage of a level is or is at least (DS)^{nc}, wherein DS is a diastereopurity as described in the present disclosure (e.g., 90%, 91%, 92%, 93%, 94%, 95%, 96%, 97%, 98%, 99% or 99.5% or more) and nc is the number of chirally controlled internucleotidic linkages as described in the present disclosure (e.g., 1-50, 1-40, 1-30, 1-25, 1-20, 5-50, 5-40, 5-30, 5-25, 5-20, 1,2, 3, 4, 5, 6, 7, 8, 9, 10, 11, 12, 13, 14, 15, 16, 17, 18, 19, 20, 21, 22, 23, 24, 25 or more). In certain embodiments, a percentage of a level is or is at least (DS)^{nc}, wherein DS is 95%-100%. For example, when DS is 99% and nc is 10, the percentage is or is at least 90% ((99%)¹⁰ 0.90=90%). In certain embodiments, level of a plurality of oligonucleotides in a composition is represented as the product of the diastereopurity of each chirally controlled internucleotidic linkage in the oligonucleotides. In certain embodiments, diastereopurity of an internucleotidic linkage connecting two nucleosides in an oligonucleotide (or nucleic acid) is represented by the diastereopurity of an internucleotidic linkage of a dimer connecting the same two nucleosides, wherein the dimer is prepared using comparable conditions, in some instances, identical synthetic cycle conditions (e.g., for the linkage between Nx and Ny in an oligonucleotide . . . NxNy . . . , the dimer is NxNy). In certain embodiments, not all chiral internucleotidic linkages are chiral controlled internucleotidic linkages, and the composition is a partially chirally controlled oligonucleotide composition. In certain embodiments, a non-chirally controlled internucleotidic linkage has a diastereopurity of less than about 80%, 75%, 70%, 65%, 60%, 55%, or of about 50%, as typically observed in stereorandom oligonucleotide compositions (e.g., as appreciated by those skilled in the art, from traditional oligonucleotide synthesis, e.g., the phosphoramidite method). In certain embodiments, oligonucleotides (or nucleic acids) of a plurality are of the same type. In certain embodiments, a chirally controlled oligonucleotide composition comprises non-random or controlled levels of individual oligonucleotide or nucleic acids types. For instance, in certain embodiments a chirally controlled oligonucleotide composition comprises one and no more than one oligonucleotide type. In certain embodiments, a chirally controlled oligonucleotide composition comprises more than one oligonucleotide type. In certain embodiments, a chirally controlled oligonucleotide composition comprises multiple oligonucleotide types. In certain embodiments, a chirally controlled oligonucleotide composition is a composition of oligonucleotides of an oligonucleotide type, which composition comprises a non-random or controlled level of a plurality of oligonucleotides of the oligonucleotide type.

Comparable: The term “comparable” is used herein to describe two (or more) sets of conditions or circumstances that are sufficiently similar to one another to permit comparison of results obtained or phenomena observed. In certain embodiments, comparable sets of conditions or circumstances are characterized by a plurality of substantially identical features and one or a small number of varied features. Those of ordinary skill in the art will appreciate that sets of conditions are comparable to one another when characterized by a sufficient number and type of substantially identical features to warrant a reasonable conclusion that differences in results obtained or phenomena observed under the different sets of conditions or circumstances are caused by or indicative of the variation in those features that are varied.

Cycloaliphatic: The term “cycloaliphatic,” “carbocycle,” “carbocyclyl,” “carbocyclic radical,” and “carbocyclic ring,” are used interchangeably, and as used herein, refer to saturated or partially unsaturated, but non-aromatic, cyclic aliphatic monocyclic, bicyclic, or polycyclic ring systems, as described herein, having, unless otherwise specified, from 3 to 30 ring members. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In certain embodiments, a cycloaliphatic group has 3-6 carbons. In certain embodiments, a cycloaliphatic group is saturated and is cycloalkyl. The term “cycloaliphatic” may also include aliphatic rings that are fused to one or more aromatic or nonaromatic rings, such as decahydronaphthyl or tetrahydronaphthyl. In certain embodiments, a cycloaliphatic group is bicyclic. In certain embodiments, a cycloaliphatic group is tricyclic. In certain embodiments, a cycloaliphatic group is polycyclic. In certain embodiments, “cycloaliphatic” refers to C₃-C₆ monocyclic hydrocarbon, or C₈-C₁₀ bicyclic or polycyclic hydrocarbon, that is completely saturated or that contains one or more units of unsaturation, but which is not aromatic, that has a single point of attachment to the rest of the molecule, or a C₉-C₁₆ polycyclic hydrocarbon that is completely saturated or that contains one or more units of unsaturation, but which is not aromatic, that has a single point of attachment to the rest of the molecule.

Heteroaliphatic: The term “heteroaliphatic”, as used herein, is given its ordinary meaning in the art and refers to aliphatic groups as described herein in which one or more carbon atoms are independently replaced with one or more heteroatoms (e.g., oxygen, nitrogen, sulfur, silicon, phosphorus, and the like). In certain embodiments, one or more units selected from C, CH, CH₂, and CH₃ are independently replaced by one or more heteroatoms (including oxidized and/or substituted forms thereof). In certain embodiments, a heteroaliphatic group is heteroalkyl. In certain embodiments, a heteroaliphatic group is heteroalkenyl.

Heteroalkyl: The term “heteroalkyl”, as used herein, is given its ordinary meaning in the art and refers to alkyl groups as described herein in which one or more carbon atoms are independently replaced with one or more heteroatoms (e.g., oxygen, nitrogen, sulfur, silicon, phosphorus, and the like). Examples of heteroalkyl groups include, but are not limited to, alkoxy, poly(ethylene glycol)-, alkyl-substituted amino, tetrahydrofuranyl, piperidinyl, morpholinyl, etc.

Heteroaryl: The terms “heteroaryl” and “heteroar-”, as used herein, used alone or as part of a larger moiety, e.g., “heteroaralkyl,” or “heteroaralkoxy,” refer to monocyclic, bicyclic or polycyclic ring systems having a total of five to thirty ring members, wherein at least one ring in the system is aromatic and at least one aromatic ring atom is a heteroatom. In certain embodiments, a heteroaryl group is a group having 5 to 10 ring atoms (i.e., monocyclic, bicyclic or polycyclic), in certain embodiments 5, 6, 9, or 10 ring atoms. In certain embodiments, each monocyclic ring unit is aromatic. In certain embodiments, a heteroaryl group has 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In certain embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom”, as used herein, means an atom that is not carbon or hydrogen. In certain embodiments, a heteroatom is boron, oxygen, sulfur, nitrogen, phosphorus, or silicon (including oxidized forms of nitrogen, sulfur, phosphorus, or silicon; charged forms of nitrogen (e.g., quaternized forms, forms as in iminium groups, etc.), phosphorus, sulfur, oxygen; etc.). In certain embodiments, a heteroatom is silicon, phosphorus, oxygen, sulfur or nitrogen. In certain embodiments, a heteroatom is silicon, oxygen, sulfur or nitrogen. In certain embodiments, a heteroatom is oxygen, sulfur or nitrogen.

Heterocycle: As used herein, the terms “heterocycle,” “heterocyclyl,” “heterocyclic radical,” and “heterocyclic ring”, as used herein, are used interchangeably and refer to a monocyclic, bicyclic or polycyclic ring moiety (e.g., 3-30 membered) that is saturated or partially unsaturated and has one or more heteroatom ring atoms. In certain embodiments, a heterocyclyl group is a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur and nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

Identity: As used herein, the term “identity” refers to the overall relatedness between polymeric molecules, e.g., between nucleic acid molecules (e.g., oligonucleotides, DNA, RNA, etc.) and/or between polypeptide molecules. In certain embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Internucleotidic linkage: As used herein, the phrase “internucleotidic linkage” refers generally to a linkage linking nucleoside units of an oligonucleotide or a nucleic acid. In certain embodiments, an internucleotidic linkage is a phosphodiester linkage, as extensively found in naturally occurring DNA and RNA molecules (natural phosphate linkage (—OP(═O)(OH)O—), which as appreciated by those skilled in the art may exist as a salt form). In certain embodiments, an internucleotidic linkage is a modified internucleotidic linkage (not a natural phosphate linkage). In certain embodiments, an internucleotidic linkage is a “modified internucleotidic linkage” wherein at least one oxygen atom or —OH of a phosphodiester linkage is replaced by a different organic or inorganic moiety. In certain embodiments, such an organic or inorganic moiety is selected from ═S, ═Se, ═NR′, —SR′, —SeR′, —N(R′)₂, B(R′)₃, —S—, —Se—, and —N(R′)—, wherein each R′ is independently as defined and described in the present disclosure. In certain embodiments, an internucleotidic linkage is a phosphotriester linkage, phosphorothioate linkage (or phosphorothioate diester linkage, —OP(═O)(SH)O—, which as appreciated by those skilled in the art may exist as a salt form), or phosphorothioate triester linkage. In certain embodiments, a modified internucleotidic linkage is a phosphorothioate linkage. In certain embodiments, an internucleotidic linkage is one of, e.g., PNA (peptide nucleic acid) or PMO (phosphorodiamidate Morpholino oligomer) linkage. In certain embodiments, a modified internucleotidic linkage is a non-negatively charged internucleotidic linkage. In certain embodiments, a modified internucleotidic linkage is a neutral internucleotidic linkage (e.g., n001 in certain provided oligonucleotides). It is understood by a person of ordinary skill in the art that an internucleotidic linkage may exist as an anion or cation at a given pH due to the existence of acid or base moieties in the linkage. In certain embodiments, a modified internucleotidic linkages is a modified internucleotidic linkages designated as s, s1, s2, s3, s4, s5, s6, s7, s8, s9, s10, s11, s12, s13, s14, s15, s16, s17 and s18 as described in WO 2017/210647.

In vitro: As used herein, the term “in vitro” refers to events that occur in an artificial environment, e.g., in a test tube or reaction vessel, in cell culture, etc., rather than within an organism (e.g., animal, plant and/or microbe).

In vivo: As used herein, the term “in vivo” refers to events that occur within an organism (e.g., animal, plant and/or microbe).

Linkage phosphorus: as defined herein, the phrase “linkage phosphorus” is used to indicate that the particular phosphorus atom being referred to is the phosphorus atom present in the internucleotidic linkage, which phosphorus atom corresponds to the phosphorus atom of a phosphodiester internucleotidic linkage as occurs in naturally occurring DNA and RNA. In certain embodiments, a linkage phosphorus atom is in a modified internucleotidic linkage, wherein each oxygen atom of a phosphodiester linkage is optionally and independently replaced by an organic or inorganic moiety. In certain embodiments, a linkage phosphorus atom is chiral (e.g., as in phosphorothioate internucleotidic linkages). In certain embodiments, a linkage phosphorus atom is achiral (e.g., as in natural phosphate linkages).

Modified nucleobase: The terms “modified nucleobase”, “modified base” and the like refer to a chemical moiety which is chemically distinct from a nucleobase, but which is capable of performing at least one function of a nucleobase. In certain embodiments, a modified nucleobase is a nucleobase which comprises a modification. In certain embodiments, a modified nucleobase is capable of at least one function of a nucleobase, e.g., forming a moiety in a polymer capable of base-pairing to a nucleic acid comprising an at least complementary sequence of bases. In certain embodiments, a modified nucleobase is substituted A, T, C, G, or U, or a substituted tautomer of A, T, C, G, or U. In certain embodiments, a modified nucleobase in the context of oligonucleotides refer to a nucleobase that is not A, T, C, G or U.

Modified nucleoside: The term “modified nucleoside” refers to a moiety derived from or chemically similar to a natural nucleoside, but which comprises a chemical modification which differentiates it from a natural nucleoside. Non-limiting examples of modified nucleosides include those which comprise a modification at the base and/or the sugar. Non-limiting examples of modified nucleosides include those with a 2′ modification at a sugar. Non-limiting examples of modified nucleosides also include abasic nucleosides (which lack a nucleobase). In certain embodiments, a modified nucleoside is capable of at least one function of a nucleoside, e.g., forming a moiety in a polymer capable of base-pairing to a nucleic acid comprising an at least complementary sequence of bases.

Modified nucleotide: The term “modified nucleotide” includes any chemical moiety which differs structurally from a natural nucleotide but is capable of performing at least one function of a natural nucleotide. In certain embodiments, a modified nucleotide comprises a modification at a sugar, base and/or internucleotidic linkage. In certain embodiments, a modified nucleotide comprises a modified sugar, modified nucleobase and/or modified internucleotidic linkage. In certain embodiments, a modified nucleotide is capable of at least one function of a nucleotide, e.g., forming a subunit in a polymer capable of base-pairing to a nucleic acid comprising an at least complementary sequence of bases.

Modified sugar: The term “modified sugar” refers to a moiety that can replace a sugar. A modified sugar mimics the spatial arrangement, electronic properties, or some other physicochemical property of a sugar. In certain embodiments, as described in the present disclosure, a modified sugar is substituted ribose or deoxyribose. In certain embodiments, a modified sugar comprises a 2′-modification. Examples of useful 2′-modification are widely utilized in the art and described herein. In certain embodiments, a 2′-modification is 2′-F. In certain embodiments, a 2′-modification is 2′-OR, wherein R is optionally substituted C₁₋₁₀ aliphatic. In certain embodiments, a 2′-modification is 2′-OMe. In certain embodiments, a 2′-modification is 2′-MOE. In certain embodiments, a modified sugar is a bicyclic sugar (e.g., a sugar used in LNA, BNA, etc.). In certain embodiments, in the context of oligonucleotides, a modified sugar is a sugar that is not ribose or deoxyribose as typically found in natural RNA or DNA.

Nucleic acid: The term “nucleic acid”, as used herein, includes any nucleotides and polymers thereof. The term “polynucleotide”, as used herein, refers to a polymeric form of nucleotides of any length, either ribonucleotides (RNA) or deoxyribonucleotides (DNA) or a combination thereof. These terms refer to the primary structure of the molecules and, thus, include double- and single-stranded DNA, and double- and single-stranded RNA. These terms include, as equivalents, analogs of either RNA or DNA comprising modified nucleotides and/or modified polynucleotides, such as, though not limited to, methylated, protected and/or capped nucleotides or polynucleotides. The terms encompass poly- or oligo-ribonucleotides (RNA) and poly- or oligo-deoxyribonucleotides (DNA); RNA or DNA derived from N-glycosides or C-glycosides of nucleobases and/or modified nucleobases; nucleic acids derived from sugars and/or modified sugars; and nucleic acids derived from phosphate bridges and/or modified internucleotidic linkages. The term encompasses nucleic acids containing any combinations of nucleobases, modified nucleobases, sugars, modified sugars, phosphate bridges or modified internucleotidic linkages. Examples include, and are not limited to, nucleic acids containing ribose moieties, nucleic acids containing deoxy-ribose moieties, nucleic acids containing both ribose and deoxyribose moieties, nucleic acids containing ribose and modified ribose moieties. Unless otherwise specified, the prefix poly- refers to a nucleic acid containing 2 to about 10,000 nucleotide monomer units and wherein the prefix oligo-refers to a nucleic acid containing 2 to about 200 nucleotide monomer units.

Nucleobase: The term “nucleobase” refers to the parts of nucleic acids that are involved in the hydrogen-bonding that binds one nucleic acid strand to another complementary strand in a sequence specific manner. The most common naturally-occurring nucleobases are adenine (A), guanine (G), uracil (U), cytosine (C), and thymine (T). In certain embodiments, a naturally-occurring nucleobases are modified adenine, guanine, uracil, cytosine, or thymine. In certain embodiments, a naturally-occurring nucleobases are methylated adenine, guanine, uracil, cytosine, or thymine. In certain embodiments, a nucleobase comprises a heteroaryl ring wherein a ring atom is nitrogen, and when in a nucleoside, the nitrogen is bonded to a sugar moiety. In certain embodiments, a nucleobase comprises a heterocyclic ring wherein a ring atom is nitrogen, and when in a nucleoside, the nitrogen is bonded to a sugar moiety. In certain embodiments, a nucleobase is a “modified nucleobase,” a nucleobase other than adenine (A), guanine (G), uracil (U), cytosine (C), and thymine (T). In certain embodiments, a modified nucleobase is substituted A, T, C, G or U. In certain embodiments, a modified nucleobase is a substituted tautomer of A, T, C, G, or U. In certain embodiments, a modified nucleobase is methylated adenine, guanine, uracil, cytosine, or thymine. In certain embodiments, a modified nucleobase mimics the spatial arrangement, electronic properties, or some other physicochemical property of the nucleobase and retains the property of hydrogen-bonding that binds one nucleic acid strand to another in a sequence specific manner. In certain embodiments, a modified nucleobase can pair with all of the five naturally occurring bases (uracil, thymine, adenine, cytosine, or guanine) without substantially affecting the melting behavior, recognition by intracellular enzymes or activity of the oligonucleotide duplex. As used herein, the term “nucleobase” also encompasses structural analogs used in lieu of natural or naturally-occurring nucleotides, such as modified nucleobases and nucleobase analogs. In certain embodiments, a nucleobase is optionally substituted A, T, C, G, or U, or an optionally substituted tautomer of A, T, C, G, or U. In certain embodiments, a “nucleobase” refers to a nucleobase unit in an oligonucleotide or a nucleic acid (e.g., A, T, C, G or U as in an oligonucleotide or a nucleic acid).

Nucleoside: The term “nucleoside” refers to a moiety wherein a nucleobase or a modified nucleobase is covalently bound to a sugar or a modified sugar. In certain embodiments, a nucleoside is a natural nucleoside, e.g., adenosine, deoxyadenosine, guanosine, deoxyguanosine, thymidine, uridine, cytidine, or deoxycytidine. In certain embodiments, a nucleoside is a modified nucleoside, e.g., a substituted natural nucleoside selected from adenosine, deoxyadenosine, guanosine, deoxyguanosine, thymidine, uridine, cytidine, and deoxycytidine. In certain embodiments, a nucleoside is a modified nucleoside, e.g., a substituted tautomer of a natural nucleoside selected from adenosine, deoxyadenosine, guanosine, deoxyguanosine, thymidine, uridine, cytidine, and deoxycytidine. In certain embodiments, a “nucleoside” refers to a nucleoside unit in an oligonucleotide or a nucleic acid.

Nucleotide: The term “nucleotide” as used herein refers to a monomeric unit of a polynucleotide that consists of a nucleobase, a sugar, and one or more internucleotidic linkages (e.g., phosphate linkages in natural DNA and RNA). The naturally occurring bases [guanine, (G), adenine, (A), cytosine, (C), thymine, (T), and uracil (U)] are derivatives of purine or pyrimidine, though it should be understood that naturally and non-naturally occurring base analogs are also included. The naturally occurring sugar is the pentose (five-carbon sugar) deoxyribose (which forms DNA) or ribose (which forms RNA), though it should be understood that naturally and non-naturally occurring sugar analogs are also included. Nucleotides are linked via internucleotidic linkages to form nucleic acids, or polynucleotides. Many internucleotidic linkages are known in the art (such as, though not limited to, phosphate, phosphorothioates, boranophosphates and the like). Artificial nucleic acids include PNAs (peptide nucleic acids), phosphotriesters, phosphorothionates, H-phosphonates, phosphoramidates, boranophosphates, methylphosphonates, phosphonoacetates, thiophosphonoacetates and other variants of the phosphate backbone of native nucleic acids, such as those described herein. In certain embodiments, a natural nucleotide comprises a naturally occurring base, sugar and internucleotidic linkage. As used herein, the term “nucleotide” also encompasses structural analogs used in lieu of natural or naturally-occurring nucleotides, such as modified nucleotides and nucleotide analogs. In certain embodiments, a “nucleotide” refers to a nucleotide unit in an oligonucleotide or a nucleic acid.

Oligonucleotide: The term “oligonucleotide” refers to a polymer or oligomer of nucleotides, and may contain any combination of natural and non-natural nucleobases, sugars, and internucleotidic linkages.

Oligonucleotides can be single-stranded or double-stranded. A single-stranded oligonucleotide can have double-stranded regions (formed by two portions of the single-stranded oligonucleotide) and a double-stranded oligonucleotide, which comprises two oligonucleotide chains, can have single-stranded regions for example, at regions where the two oligonucleotide chains are not complementary to each other. Example oligonucleotides include, but are not limited to structural genes, genes including control and termination regions, self-replicating systems such as viral or plasmid DNA, single-stranded and double-stranded RNAi agents and other RNA interference reagents (RNAi agents or iRNA agents), shRNA, antisense oligonucleotides, ribozymes, microRNAs, microRNA mimics, supermirs, aptamers, antimirs, antagomirs, Ul adaptors, triplex-forming oligonucleotides, G-quadruplex oligonucleotides, RNA activators, immuno-stimulatory oligonucleotides, and decoy oligonucleotides.

Oligonucleotides of the present disclosure can be of various lengths. In particular embodiments, oligonucleotides can range from about 2 to about 200 nucleosides in length. In various related embodiments, oligonucleotides, single-stranded, double-stranded, or triple-stranded, can range in length from about 4 to about 10 nucleosides, from about 10 to about 50 nucleosides, from about 20 to about 50 nucleosides, from about 15 to about 30 nucleosides, from about 20 to about 30 nucleosides in length. In certain embodiments, the oligonucleotide is from about 9 to about 39 nucleosides in length. In certain embodiments, the oligonucleotide is at least 4, 5, 6, 7, 8, 9, 10, 11, 12, 13, 14, 15, 16, 17, 18, 19, 20, 21, 22, 23, 24, or 25 nucleosides in length. In certain embodiments, the oligonucleotide is at least 4 nucleosides in length. In certain embodiments, the oligonucleotide is at least 5 nucleosides in length. In certain embodiments, the oligonucleotide is at least 6 nucleosides in length. In certain embodiments, the oligonucleotide is at least 7 nucleosides in length. In certain embodiments, the oligonucleotide is at least 8 nucleosides in length. In certain embodiments, the oligonucleotide is at least 9 nucleosides in length. In certain embodiments, the oligonucleotide is at least 10 nucleosides in length. In certain embodiments, the oligonucleotide is at least 11 nucleosides in length. In certain embodiments, the oligonucleotide is at least 12 nucleosides in length. In certain embodiments, the oligonucleotide is at least 15 nucleosides in length. In certain embodiments, the oligonucleotide is at least 15 nucleosides in length. In certain embodiments, the oligonucleotide is at least 16 nucleosides in length. In certain embodiments, the oligonucleotide is at least 17 nucleosides in length. In certain embodiments, the oligonucleotide is at least 18 nucleosides in length. In certain embodiments, the oligonucleotide is at least 19 nucleosides in length. In certain embodiments, the oligonucleotide is at least 20 nucleosides in length. In certain embodiments, the oligonucleotide is at least 25 nucleosides in length. In certain embodiments, the oligonucleotide is at least 30 nucleosides in length. In certain embodiments, each nucleoside counted in an oligonucleotide length independently comprises a nucleobase comprising a ring having at least one nitrogen ring atom. In certain embodiments, each nucleoside counted in an oligonucleotide length independently comprises A, T, C, G, or U, or optionally substituted A, T, C, G, or U, or an optionally substituted tautomer of A, T, C, G or U.

Oligonucleotide type: As used herein, the phrase “oligonucleotide type” is used to define an oligonucleotide that has a particular base sequence, pattern of backbone linkages (i.e., pattern of internucleotidic linkage types, for example, phosphate, phosphorothioate, phosphorothioate triester, etc.), pattern of backbone chiral centers (i.e., pattern of linkage phosphorus stereochemistry (Rp/Sp)), and pattern of backbone phosphorus modifications. In certain embodiments, oligonucleotides of a common designated “type” are structurally identical to one another.

One of skill in the art will appreciate that synthetic methods of the present disclosure provide for a degree of control during the synthesis of an oligonucleotide strand such that each nucleotide unit of the oligonucleotide strand can be designed and/or selected in advance to have a particular stereochemistry at the linkage phosphorus and/or a particular modification at the linkage phosphorus, and/or a particular base, and/or a particular sugar. In certain embodiments, an oligonucleotide strand is designed and/or selected in advance to have a particular combination of stereocenters at the linkage phosphorus. In certain embodiments, an oligonucleotide strand is designed and/or determined to have a particular combination of modifications at the linkage phosphorus. In certain embodiments, an oligonucleotide strand is designed and/or selected to have a particular combination of bases. In certain embodiments, an oligonucleotide strand is designed and/or selected to have a particular combination of one or more of the above structural characteristics. In certain embodiments, the present disclosure provides compositions comprising or consisting of a plurality of oligonucleotide molecules (e.g., chirally controlled oligonucleotide compositions). In certain embodiments, all such molecules are of the same type (i.e., are structurally identical to one another). In certain embodiments, however, provided compositions comprise a plurality of oligonucleotides of different types, typically in pre-determined relative amounts.

Optionally Substituted: As described herein, compounds, e.g., oligonucleotides, of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. In certain embodiments, an optionally substituted group is unsubstituted. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. Certain substituents are described below.

Suitable monovalent substituents on a substitutable atom, e.g., a suitable carbon atom, are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, —SC(S)SR^{∘}; —(CH₂)₀₋₄SC(O)R^{∘}; —(CH₂)₀₋₄C(O)NR⁰²; —C(S)NR^{∘}₂; —C(S)SR^{∘}; —(CH₂)₀₋₄OC(O)NR⁰²; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —Si(R^{∘})₃; —OSi(R^{∘})₃; —B(R^{∘})₂; —OB(R^{∘})₂; —OB(OR^{∘})₂; —P(R^{∘})₂; —P(OR^{∘})₂; —P(R^{∘})(OR^{∘}); —OP(R^{∘})₂; —OP(OR^{∘})₂; —OP(R^{∘})(OR^{∘}); —P(O)(R^{∘})₂; —P(O)(OR^{∘})₂; —OP(O)(R^{∘})₂; —OP(O)(OR^{∘})₂; —OP(O)(OR^{∘})(SR^{∘}); —SP(O)(R^{∘})₂; —SP(O)(OR^{∘})₂; —N(R^{∘})P(O)(R^{∘})₂; —N(R^{∘})P(O)(OR^{∘})₂; —P(R^{∘})₂[B(R^{∘})₃]; —P(OR^{∘})₂[B(R^{∘})₃]; —OP(R^{∘})₂[B(R^{∘})₃]; —OP(OR^{∘})₂[B(R^{∘})₃]; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined herein and is independently hydrogen, C₁₋₂₀ aliphatic, C₁₋₂₀ heteroaliphatic having 1-5 heteroatoms independently selected from nitrogen, oxygen, sulfur, silicon and phosphorus, —CH₂—(C₆₋₁₄ aryl), —O(CH₂)₀₋₁(C₆₋₁₄ aryl), —CH₂-(5-14 membered heteroaryl ring), a 5-20 membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R^{∘}, taken together with their intervening atom(s), form a 5-20 membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, and a 5-6-membered saturated, partially unsaturated, or aryl ring having 0-4 heteroatoms independently selected from nitrogen, oxygen, and sulfur. Suitable divalent substituents on a saturated carbon atom of R^{∘} include ═O and ═S.

Suitable divalent substituents, e.g., on a suitable carbon atom, are independently the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, and an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, and an unsubstituted 5-6-membered saturated, partially unsaturated, and aryl ring having 0-4 heteroatoms independently selected from nitrogen, oxygen, and sulfur.

Suitable substituents on the aliphatic group of R* are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

In certain embodiments, suitable substituents on a substitutable nitrogen are independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, and sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁ Ph, or a 5-6-membered saturated, partially unsaturated, or aryl ring having 0-4 heteroatoms independently selected from nitrogen, oxygen, and sulfur.

P-modification: as used herein, the term “P-modification” refers to any modification at the linkage phosphorus other than a stereochemical modification. In certain embodiments, a P-modification comprises addition, substitution, or removal of a pendant moiety covalently attached to a linkage phosphorus.

Partially unsaturated: As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

Pharmaceutical composition: As used herein, the term “pharmaceutical composition” refers to an active agent, formulated together with one or more pharmaceutically acceptable carriers. In certain embodiments, an active agent is present in unit dose amount appropriate for administration in a therapeutic regimen that shows a statistically significant probability of achieving a predetermined therapeutic effect when administered to a relevant population. In certain embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In certain embodiments, pharmaceutically acceptable salt include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In certain embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In certain embodiments, a provided compound comprises one or more acidic groups, e.g., an oligonucleotide, and a pharmaceutically acceptable salt is an alkali, alkaline earth metal, or ammonium (e.g., an ammonium salt of N(R)₃, wherein each R is independently defined and described in the present disclosure) salt. Representative alkali or alkaline earth metal salts include sodium, lithium, potassium, calcium, magnesium, and the like. In certain embodiments, a pharmaceutically acceptable salt is a sodium salt. In certain embodiments, a pharmaceutically acceptable salt is a potassium salt. In certain embodiments, a pharmaceutically acceptable salt is a calcium salt. In certain embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. In certain embodiments, a provided compound comprises more than one acid groups, for example, an oligonucleotide may comprise two or more acidic groups (e.g., in natural phosphate linkages and/or modified internucleotidic linkages). In certain embodiments, a pharmaceutically acceptable salt, or generally a salt, of such a compound comprises two or more cations, which can be the same or different. In certain embodiments, in a pharmaceutically acceptable salt (or generally, a salt), all ionizable hydrogen (e.g., in an aqueous solution with a pKa no more than about 11, 10, 9, 8, 7, 6, 5, 4, 3, or 2; in certain embodiments, no more than about 7; in certain embodiments, no more than about 6; in certain embodiments, no more than about 5; in certain embodiments, no more than about 4; in certain embodiments, no more than about 3) in the acidic groups are replaced with cations. In certain embodiments, each phosphorothioate and phosphate group independently exists in its salt form (e.g., if sodium salt, —O—P(O)(SNa)—O— and —O—P(O)(ONa)—O—, respectively). In certain embodiments, each phosphorothioate and phosphate internucleotidic linkage independently exists in its salt form (e.g., if sodium salt, —O—P(O)(SNa)—O— and —O—P(O)(ONa)—O—, respectively). In certain embodiments, a pharmaceutically acceptable salt is a sodium salt of an oligonucleotide. In certain embodiments, a pharmaceutically acceptable salt is a sodium salt of an oligonucleotide, wherein each acidic phosphate and modified phosphate group (e.g., phosphorothioate, phosphate, etc.), if any, exists as a salt form (all sodium salt).

Predetermined: By predetermined (or pre-determined) is meant deliberately selected or non-random or controlled, for example as opposed to randomly occurring, random, or achieved without control. Those of ordinary skill in the art, reading the present specification, will appreciate that the present disclosure provides technologies that permit selection of particular chemistry and/or stereochemistry features to be incorporated into oligonucleotide compositions, and further permits controlled preparation of oligonucleotide compositions having such chemistry and/or stereochemistry features. Such provided compositions are “predetermined” as described herein. Compositions that may contain certain oligonucleotides because they happen to have been generated through a process that are not controlled to intentionally generate the particular chemistry and/or stereochemistry features are not “predetermined” compositions. In certain embodiments, a predetermined composition is one that can be intentionally reproduced (e.g., through repetition of a controlled process). In certain embodiments, a predetermined level of a plurality of oligonucleotides in a composition means that the absolute amount, and/or the relative amount (ratio, percentage, etc.) of the plurality of oligonucleotides in the composition is controlled. In certain embodiments, a predetermined level of a plurality of oligonucleotides in a composition is achieved through chirally controlled oligonucleotide preparation.

Protecting group: The term “protecting group,” as used herein, is well known in the art and includes those described in detail in Protecting Groups in Organic Synthesis, T. W. Greene and P. G. M. Wuts, 3^{rd} edition, John Wiley & Sons, 1999, the entirety of which is incorporated herein by reference. Also included are those protecting groups specially adapted for nucleoside and nucleotide chemistry described in Current Protocols in Nucleic Acid Chemistry, edited by Serge L. Beaucage et al. 06/2012, the entirety of Chapter 2 is incorporated herein by reference. Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N−2,3-diphenylmaleimide, N−2,5-dimethylpyrrole, N−1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N−[2-(trimethylsilyl)ethoxy]methylamine (SEM), N−3-acetoxypropylamine, N−(1-isopropyl-4-nitro-2-oxo-3-pyroolin-3-yl)amine, quaternary ammonium salts, N-benzylamine, N-di(4-methoxyphenyl)methylamine, N−5-dibenzosuberylamine, N-triphenylmethylamine (Tr), N−[(4-methoxyphenyl)diphenylmethyl]amine (MMTr), N−9-phenylfluorenylamine (PhF), N−2,7-dichloro-9-fluorenylmethyleneamine, N-ferrocenylmethylamino (Fcm), N-2-picolylamino N′-oxide, N−1,1-dimethylthiomethyleneamine, N-benzylideneamine, N-p-methoxybenzylideneamine, N-diphenylmethyleneamine, N−[(2-pyridyl)mesityl]methyleneamine, N−(N′,N′-dimethylaminomethylene)amine, N,N′-isopropylidenediamine, N-p-nitrobenzylideneamine, N-salicylideneamine, N−5-chlorosalicylideneamine, N−(5-chloro-2-hydroxyphenyl)phenylmethyleneamine, N-cyclohexylideneamine, N−(5,5-dimethyl-3-oxo-1-cyclohexenyl)amine, N-borane derivative, N-diphenylborinic acid derivative, N−[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), 0-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Suitably protected carboxylic acids further include, but are not limited to, silyl-, alkyl-, alkenyl-, aryl-, and arylalkyl-protected carboxylic acids. Examples of suitable silyl groups include trimethylsilyl, triethylsilyl, t-butyldimethylsilyl, t-butyldiphenylsilyl, triisopropylsilyl, and the like. Examples of suitable alkyl groups include methyl, benzyl, p-methoxybenzyl, 3,4-dimethoxybenzyl, trityl, t-butyl, tetrahydropyran-2-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, O-nitrobenzyl, p-nitrobenzyl, p-halobenzyl, 2,6-dichlorobenzyl, p-cyanobenzyl), and 2- and 4-picolyl.

Suitable hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

In certain embodiments, a hydroxyl protecting group is acetyl, t-butyl, t-butoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, benzoyl, p-phenylbenzoyl, 2,6-dichlorobenzyl, diphenylmethyl, p-nitrobenzyl, triphenylmethyl (trityl), 4,4′-dimethoxytrityl, trimethylsilyl, triethylsilyl, t-butyldimethylsilyl, t-butyldiphenylsilyl, triphenylsilyl, triisopropylsilyl, benzoylformate, chloroacetyl, trichloroacetyl, trifiuoroacetyl, pivaloyl, 9-fluorenylmethyl carbonate, mesylate, tosylate, triflate, trityl, monomethoxytrityl (MMTr), 4,4′-dimethoxytrityl, (DMTr) and 4,4′,4″-trimethoxytrityl (TMTr), 2-cyanoethyl (CE or Cne), 2-(trimethylsilyl)ethyl (TSE), 2-(2-nitrophenyl)ethyl, 2-(4-cyanophenyl)ethyl 2-(4-nitrophenyl)ethyl (NPE), 2-(4-nitrophenylsulfonyl)ethyl, 3,5-dichlorophenyl, 2,4-dimethylphenyl, 2-nitrophenyl, 4-nitrophenyl, 2,4,6-trimethylphenyl, 2-(2-nitrophenyl)ethyl, butylthiocarbonyl, 4,4′,4″-tris(benzoyloxy)trityl, diphenylcarbamoyl, levulinyl, 2-(dibromomethyl)benzoyl (Dbmb), 2-(isopropylthiomethoxymethyl)benzoyl (Ptmt), 9-phenylxanthen-9-yl (pixyl) or 9-(p-methoxyphenyl)xanthine-9-yl (MOX). In certain embodiments, each of the hydroxyl protecting groups is, independently selected from acetyl, benzyl, t-butyldimethylsilyl, t-butyldiphenylsilyl and 4,4′-dimethoxytrityl. In certain embodiments, the hydroxyl protecting group is selected from the group consisting of trityl, monomethoxytrityl and 4,4′-dimethoxytrityl group. In certain embodiments, a phosphorous linkage protecting group is a group attached to the phosphorous linkage (e.g., an internucleotidic linkage) throughout oligonucleotide synthesis. In certain embodiments, a protecting group is attached to a sulfur atom of an phosphorothioate group. In certain embodiments, a protecting group is attached to an oxygen atom of an internucleotide phosphorothioate linkage. In certain embodiments, a protecting group is attached to an oxygen atom of the internucleotide phosphate linkage. In certain embodiments a protecting group is 2-cyanoethyl (CE or Cne), 2-trimethylsilylethyl, 2-nitroethyl, 2-sulfonylethyl, methyl, benzyl, o-nitrobenzyl, 2-(p-nitrophenyl)ethyl (NPE or Npe), 2-phenylethyl, 3-(N-tert-butylcarboxamido)-1-propyl, 4-oxopentyl, 4-methylthio-1-butyl, 2-cyano-1,1-dimethylethyl, 4-N-methylaminobutyl, 3-(2-pyridyl)-1-propyl, 2-[N-methyl-N−(2-pyridyl)]aminoethyl, 2-(N-formyl,N-methyl)aminoethyl, or 4-[N-methyl-N−(2,2,2-trifluoroacetyl)amino]butyl.

Subject: As used herein, the term “subject” or “test subject” refers to any organism to which a compound (e.g., an oligonucleotide) or composition is administered in accordance with the present disclosure e.g., for experimental, diagnostic, prophylactic and/or therapeutic purposes. Typical subjects include animals (e.g., mammals such as mice, rats, rabbits, non-human primates, and humans; insects; worms; etc.) and plants. In certain embodiments, a subject is a human. In certain embodiments, a subject may be suffering from and/or susceptible to a disease, disorder and/or condition.

Substantially: As used herein, the term “substantially” refers to the qualitative condition of exhibiting total or near-total extent or degree of a characteristic or property of interest. A base sequence which is substantially identical or complementary to a second sequence is not fully identical or complementary to the second sequence, but is mostly or nearly identical or complementary to the second sequence. In certain embodiments, an oligonucleotide with a substantially complementary sequence to another oligonucleotide or nucleic acid forms duplex with the oligonucleotide or nucleic acid in a similar fashion as an oligonucleotide with a fully complementary sequence. In addition, one of ordinary skill in the biological and/or chemical arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Sugar: The term “sugar” refers to a monosaccharide or polysaccharide in closed and/or open form. In certain embodiments, sugars are monosaccharides. In certain embodiments, sugars are polysaccharides. Sugars include, but are not limited to, ribose, deoxyribose, pentofuranose, pentopyranose, and hexopyranose moieties. As used herein, the term “sugar” also encompasses structural analogs used in lieu of conventional sugar 5 molecules, such as glycol, polymer of which forms the backbone of the nucleic acid analog, glycol nucleic acid (“GNA”), etc. As used herein, the term “sugar” also encompasses structural analogs used in lieu of natural or naturally-occurring nucleotides, such as modified sugars and nucleotide sugars. In certain embodiments, a sugar is a RNA or DNA sugar (ribose or deoxyribose). In certain embodiments, a sugar is a modified ribose or deoxyribose sugar, e.g., 2′-modified, 5′-modified, etc. As described herein, in certain embodiments, when used in oligonucleotides and/or nucleic acids, modified sugars may provide one or more desired properties, activities, etc. In certain embodiments, a sugar is optionally substituted ribose or deoxyribose. In certain embodiments, a “sugar” refers to a sugar unit in an oligonucleotide or a nucleic acid.

Susceptible to: An individual who is “susceptible to” a disease, disorder and/or condition is one who has a higher risk of developing the disease, disorder and/or condition than does a member of the general public. In certain embodiments, an individual who is susceptible to a disease, disorder and/or condition is predisposed to have that disease, disorder and/or condition. In certain embodiments, an individual who is susceptible to a disease, disorder and/or condition may not have been diagnosed with the disease, disorder and/or condition. In certain embodiments, an individual who is susceptible to a disease, disorder and/or condition may exhibit symptoms of the disease, disorder and/or condition. In certain embodiments, an individual who is susceptible to a disease, disorder and/or condition may not exhibit symptoms of the disease, disorder and/or condition. In certain embodiments, an individual who is susceptible to a disease, disorder, and/or condition will develop the disease, disorder, and/or condition. In certain embodiments, an individual who is susceptible to a disease, disorder, and/or condition will not develop the disease, disorder, and/or condition.

Therapeutic agent: As used herein, the term “therapeutic agent” in general refers to any agent that elicits a desired effect (e.g., a desired biological, clinical, or pharmacological effect) when administered to a subject. In certain embodiments, an agent, e.g., a dsRNAi agent, is considered to be a therapeutic agent if it demonstrates a statistically significant effect across an appropriate population. In certain embodiments, an appropriate population is a population of subjects suffering from and/or susceptible to a disease, disorder or condition. In certain embodiments, an appropriate population is a population of model organisms. In certain embodiments, an appropriate population may be defined by one or more criterion such as age group, gender, genetic background, preexisting clinical conditions, prior exposure to therapy. In certain embodiments, a therapeutic agent is a substance that alleviates, ameliorates, relieves, inhibits, prevents, delays onset of, reduces severity of, and/or reduces incidence of one or more hepaticsymptoms or features of a disease, disorder, and/or condition in a subject when administered to the subject in an effective amount. In certain embodiments, a “therapeutic agent” is an agent that has been or is required to be approved by a government agency before it can be marketed for administration to humans. In certain embodiments, a “therapeutic agent” is an agent for which a medical prescription is required for administration to humans. In certain embodiments, a therapeutic agent is a provided compound, e.g., a provided oligonucleotide.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In certain embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In certain embodiments, a therapeutically effective amount is administered in a single dose; in certain embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In certain embodiments, treatment may be administered to a subject who exhibits only early signs of the disease, disorder, and/or condition, for example for the purpose of decreasing the risk of developing pathology associated with the disease, disorder, and/or condition.

Unsaturated: The term “unsaturated,” as used herein, means that a moiety has one or more units of unsaturation.

Wild-type: As used herein, the term “wild-type” has its art-understood meaning that refers to an entity having a structure and/or activity as found in nature in a “normal” (as contrasted with mutant, diseased, altered, etc.) state or context. Those of ordinary skill in the art will appreciate that wild type genes and polypeptides often exist in multiple different forms (e.g., alleles).

As those skilled in the art will appreciate, methods and compositions described herein relating to provided compounds (e.g., oligonucleotides) generally also apply to pharmaceutically acceptable salts of such compounds.

### 1. Description of Certain Embodiments

Oligonucleotides are useful tools for a wide variety of applications. For example, RNAi oligonucleotides are useful in therapeutic, diagnostic, and research applications, including the treatment of a variety of conditions, disorders, and diseases. The use of naturally occurring nucleic acids (e.g., unmodified DNA or RNA) is limited, for example, by their susceptibility to endo- and exo-nucleases. As such, various synthetic counterparts have been developed to circumvent these shortcomings and/or to further improve various properties and activities. These include synthetic oligonucleotides that contain chemical modifications, e.g., base modifications, sugar modifications, backbone modifications, etc., which, among other things, render these molecules less susceptible to degradation and improve other properties and/or activities. From a structural point of view, modifications to internucleotidic linkages can introduce chirality and/or alter charge, and certain properties may be affected by configurations of linkage phosphorus atoms of oligonucleotides. For example, binding affinity, sequence specific binding to complementary RNA, stability against nucleases, cleavage of target nucleic acids, delivery, pharmacokinetics, etc., can be affected by, inter alia, chirality and/or charge of backbone linkage atoms.

In certain embodiments, the present disclosure demonstrates that compositions comprising ds oligonucleotides (e.g., dsRNAi oligonucleotides, also referred to as dsRNAi agents) with controlled structural elements provide unexpected properties and/or activities.

In certain embodiments, the present disclosure encompasses the recognition that stereochemistry, e.g., stereochemistry of backbone chiral centers, can unexpectedly maintain or improve properties of ds oligonucleotides. In contrast to many prior observations that some structural elements that increase stability can also lower activity, for example, RNA interference, the present disclosure demonstrates that control of stereochemistry can, surprisingly, maintain increase stability while not significantly decreasing activity. For example, but not by way of limitation, the instant disclosure relates, in part. For example, but not by way of limitation, the instant disclosure relates, in part, to ds oligonucleotides comprising one or more of
(1) a guide strand comprising backbone phosphorothioate chiral centers in Sp configuration between the 3′ terminal nucleotide and the penultimate (N−1) nucleotide and as between the penultimate (N−1) nucleotide and the immediately upstream, i.e., in the 5′ direction, (N−2) nucleotide;
(2) a guide strand comprising backbone phosphorothioate chiral centers in Rp, Sp, or alternating configurations between the 5′ terminal (+1) nucleotide and the immediately downstream, i.e., in the 3′ direction, (+2) nucleotide and between the +2 nucleotide and the immediately downstream (+3) nucleotide;
(3) a guide strand comprising one or more backbone phosphorothioate chiral centers upstream, i.e., in the 5′ direction, relative to backbone phosphorothioate chiral centers in Sp configuration between the 3′ terminal nucleotide and the penultimate (N−1) nucleotide and as between the penultimate (N−1) nucleotide and the immediately upstream (N−2) nucleotide, where the upstream backbone phosphorothioate chiral centers are in Rp or Sp configuration;
(4) a guide strand comprising one or more backbone phosphorothioate chiral centers in Rp or Sp configuration between the 5′ terminal (+1) nucleotide and the immediately downstream (+2) nucleotide and between the +2 nucleotide and the immediately downstream (+3) nucleotide, as well as between one or both of: (a) the +3 nucleotide and the +4 nucleotide; and (b) the +5 nucleotide and the +6 nucleotide;
(5) a passenger strand in combination with one or more of the aforementioned guide strands, comprising one or more backbone chiral centers in Rp or Sp configuration; and
6) a passenger strand in combination with one or more of the aforementioned guide strands, comprising backbone phosphorothioate chiral centers in the Sp configuration between the 5′ terminal (+1) nucleotide and the immediately downstream, i.e., in the 3′ direction, (+2) nucleotide and between the 3′ terminal nucleotide and the penultimate (N−1) nucleotide;
   wherein the ds oligonucleotide further comprises one or more of.
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N−1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N−1) nucleotide and the 3′ terminal (N) nucleotide.

In certain embodiments, the present disclosure encompasses the recognition that stereochemistry, e.g., stereochemistry of chiral centers at a 5′ terminal modification of guide strands, can unexpectedly maintain or improve properties of ds oligonucleotides wherein the guide strand of the ds oligonucleotide also comprises a phosphorothioate chiral center in Rp or Sp configuration. For example, but not by way of limitation, the instant disclosure relates, in part, to ds oligonucleotides comprising a guide stranding comprising: (1) a phosphorothioate chiral center in Rp or Sp configuration; (2) an Rp, Sp, or stereorandom non-negatively charged internucleotidic linkage where the 3′ nucleotide of a nucleotide pair linked by an Rp, Sp, or stereorandom non-negatively charged internucleotidic linkage comprises a 2′ modification, e.g., a 2′ F; and (3) a 5′ terminal modification selected from:
(a) 5′ PO modifications, such as, but not limited to:

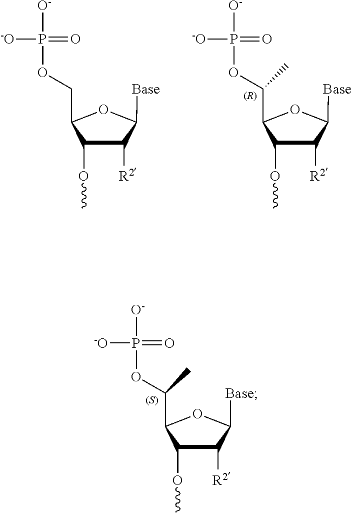

(b) 5′ VP modifications, such as, but not limited to:

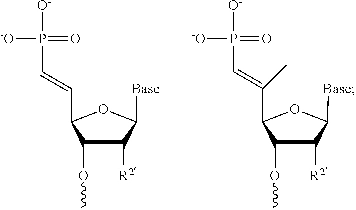

(c) 5′ MeP modifications, such as, but not limited to:

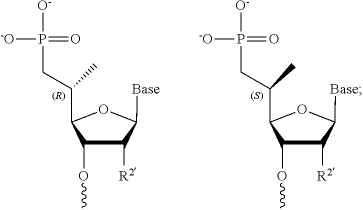

(d) 5′ PN and 5′ Trizole-P modifications, such as, but not limited to:

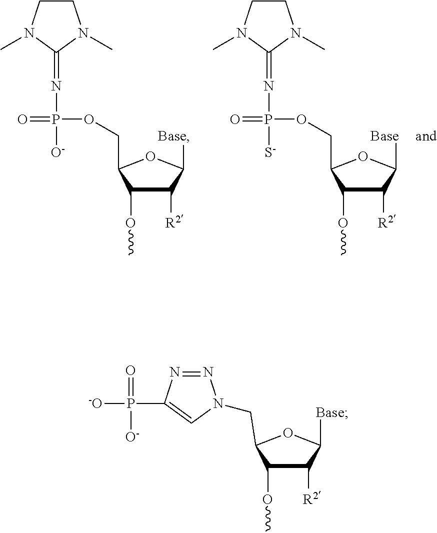

Wherein Base is selected from A, C, G, T, U, abasic and modified nucleobases; R^{2′} is selected from H, OH, O-alkyl, F, MOE, locked nucleic acid (LNA) bridges and bridged nucleic acid (BNA) bridges to the 4′ C, such as, but not limited to:

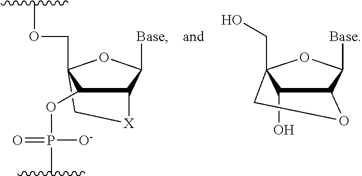

In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In certain other embodiments, the present disclosure encompasses the recognition that stereochemistry, e.g., stereochemistry of chiral centers at the 5′ terminal nucleotide of guide strands, can unexpectedly maintain or improve properties of ds oligonucleotides wherein the guide strand of the ds oligonucleotide also comprises a phosphorothioate chiral center in Rp or Sp configuration. For example, but not by way of limitation, the instant disclosure relates, in part, to ds oligonucleotides comprising a guide stranding comprising: (1) a phosphorothioate chiral center in Rp or Sp configuration; (2) an Rp, Sp, or stereorandom non-negatively charged internucleotidic linkage where the 3′ nucleotide of a nucleotide pair linked by an Rp, Sp, or stereorandom non-negatively charged internucleotidic linkage comprises a 2′ modification, e.g., a 2′ F; and (3) a 5′ terminal modification selected from:
(a) 5′ PO nucleotides, such as, but not limited to:

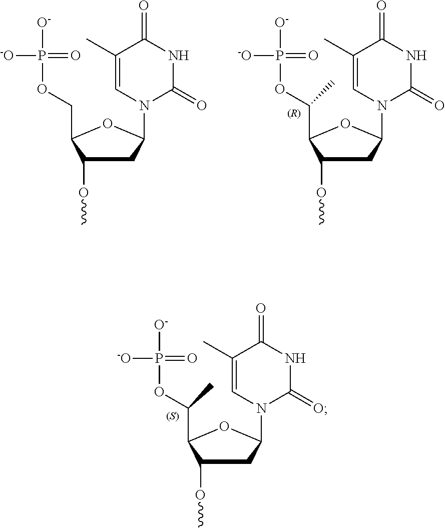

(b) 5′ VP nucleotides, such as but not limited to:

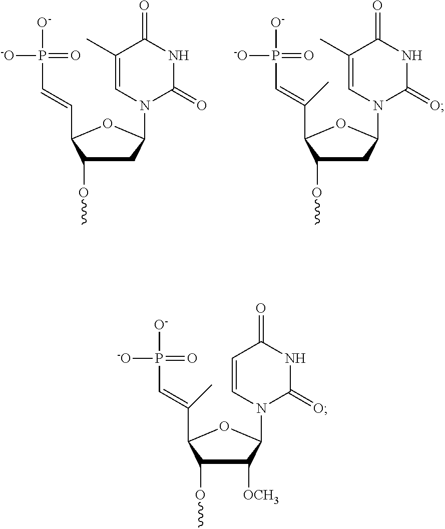

(c) 5′ MeP nucleotides, such as, but not limited to:

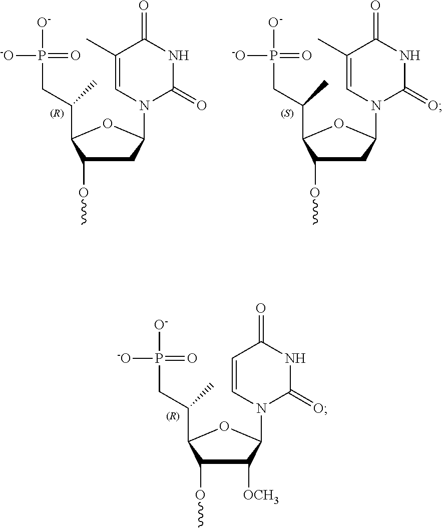

(d) 5′ PN and 5′ Trizole-P nucleotides, such as, but not limited to:

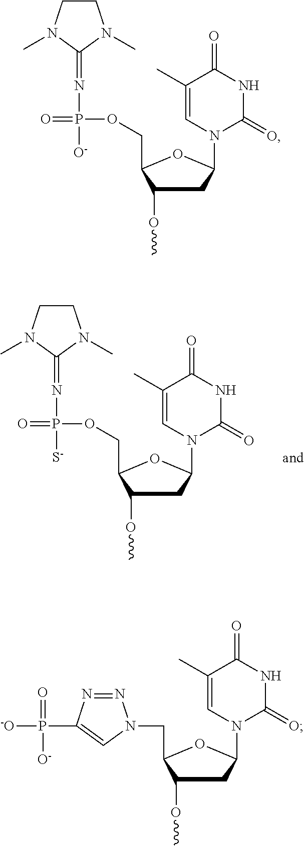

(e) 5′ abasic VP and 5′ abasic MeP nucleotides, such as, but not limited to:

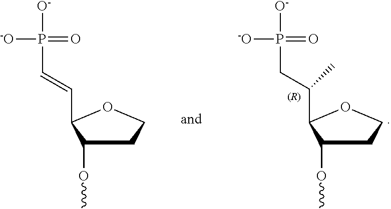

In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In certain embodiments, the present disclosure encompasses the recognition that Rp, Sp, or stereorandom non-naturally-occurring internucleotidic linkages, e.g., neutral internucleotidic linkages, can unexpectedly maintain or improve properties of ds oligonucleotides. For example, the present disclosure demonstrates that modified internucleotidic linkages can be introduced into ds oligonucleotide without significantly decreasing the activity of the ds oligonucleotide. For example, but not by way of limitation, the instant disclosure relates, in part, comprising one or more of.
(1) a guide strand comprising backbone phosphorothioate chiral centers in Sp configuration between the 3′ terminal nucleotide and the penultimate (N−1) nucleotide and as between the penultimate (N−1) nucleotide and the immediately upstream, i.e., in the 5′ direction, (N−2) nucleotide;
(2) a guide strand comprising backbone phosphorothioate chiral centers in Rp, Sp, or alternating configurations between the 5′ terminal (+1) nucleotide and the immediately downstream, i.e., in the 3′ direction, (+2) nucleotide and between the +2 nucleotide and the immediately downstream (+3) nucleotide;
(3) a guide strand comprising one or more backbone phosphorothioate chiral centers upstream, i.e., in the 5′ direction, relative to backbone phosphorothioate chiral centers in Sp configuration between the 3′ terminal nucleotide and the penultimate (N−1) nucleotide and as between the penultimate (N−1) nucleotide and the immediately upstream (N−2) nucleotide, where the upstream backbone phosphorothioate chiral centers are in Rp or Sp configuration;
(4) a guide strand comprising one or more backbone phosphorothioate chiral centers in Rp or Sp configuration between the 5′ terminal (+1) nucleotide and the immediately downstream (+2) nucleotide and between the +2 nucleotide and the immediately downstream (+3) nucleotide, as well as between one or both of: (a) the +3 nucleotide and the +4 nucleotide; and (b) the +5 nucleotide and the +6 nucleotide;
(5) a passenger strand in combination with one or more of the aforementioned guide strands, comprising one or more backbone chiral centers in Rp or Sp configuration; and
6) a passenger strand in combination with one or more of the aforementioned guide strands, comprising backbone phosphorothioate chiral centers in the Sp configuration between the 5′ terminal (+1) nucleotide and the immediately downstream, i.e., in the 3′ direction, (+2) nucleotide and between the 3′ terminal nucleotide and the penultimate (N−1) nucleotide;
   wherein the ds oligonucleotide further comprises one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N−1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand;
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N−1) nucleotide and the 3′ terminal (N) nucleotide.

In certain embodiments, the present disclosure encompasses the recognition that non-naturally occurring internucleotidic linkages, e.g., neutral internucleotidic linkages, can, in certain embodiments, be used to link one or more molecules to the double-stranded oligonucleotides described herein. In certain embodiments, such linked molecules can facilitate targeting and/or delivery of the double-stranded oligonucleotide. For example, but not limitation, such linked molecules an include lipophilic molecules. In certain embodiments, the linked molecule is a molecule comprising one or more GalNac moieties. In certain embodiments, the the linked molecule is a receptor. In certain embodiments, the linked molecule is a receptor ligand.

In certain embodiments, the present disclosure provides technologies (e.g., compounds, methods, etc.) for improving oligonucleotide stability while maintaining or increasing activity, including compositions of improved-stability oligonucleotides.

In certain embodiments, the present disclosure provides technologies for incorporating various additional chemical moieties into ds oligonucleotides. In certain embodiments, the present disclosure provides, for example, reagents and methods for introducing additional chemical moieties through nucleobases (e.g., by covalent linkage, optionally via a linker, to a site on a nucleobase).

In certain embodiments, the present disclosure provides technologies, e.g., ds oligonucleotide compositions and methods thereof, that achieve allele-specific suppression, wherein transcripts from one allele of a particular target gene is selectively knocked down relative to at least one other allele of the same gene.

Among other things, the present disclosure provides structural elements, technologies and/or features that can be incorporated into ds oligonucleotides and can impart or tune one or more properties thereof (e.g., relative to an otherwise identical ds oligonucleotide lacking the relevant technology or feature). In certain embodiments, the present disclosure documents that one or more provided technologies and/or features can usefully be incorporated into ds oligonucleotides of various sequences.

In certain embodiments, the present disclosure demonstrates that certain provided structural elements, technologies and/or features are particularly useful for ds oligonucleotides that participate in and/or direct RNAi mechanisms (e.g., RNAi agents). Regardless, however, the teachings of the present disclosure are not limited to ds oligonucleotides that participate in or operate via any particular mechanism.

In certain embodiments, the present disclosure pertains to any ds oligonucleotide, useful for any purpose, which operates through any mechanism, and which comprises any sequence, structure or format (or portion thereof) described herein.

In certain embodiments, the present disclosure provides a ds oligonucleotide, useful for any purpose, which operates through any mechanism, and which comprises any sequence, structure or format (or portion thereof) described herein, including, In certain embodiments, the guide strand comprises backbone phosphorothioate chiral centers in Sp configuration between the 3′ terminal nucleotide and the penultimate (N−1) nucleotide and as between the penultimate (N−1) nucleotide and the immediately upstream (N−2) nucleotide, and one or more of.
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N−1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N−1) nucleotide and the 3′ terminal (N) nucleotide.

In certain embodiments, the guide strand comprises backbone phosphorothioate chiral centers in Rp, Sp, or alternating configurations between the 5′ terminal (+1) nucleotide and the immediately downstream (+2) nucleotide and between the +2 nucleotide and the immediately downstream (+3) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N−1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N−1) nucleotide and the 3′ terminal (N) nucleotide.

In certain embodiments, the guide strand comprises one or more backbone phosphorothioate chiral centers in Rp or Sp configuration upstream of backbone phosphorothioate chiral centers in Sp configuration between the 3′ terminal nucleotide and the penultimate (N−1) nucleotide and as between the penultimate (N−1) nucleotide and the immediately upstream (N−2) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N−1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N−1) nucleotide and the 3′ terminal (N) nucleotide.

In certain embodiments, the guide strand comprises one or more Rp, Sp, or stereorandom non-negatively charged internucleotidic linkage occurs between the second (+2) and third (+3) nucleotides, relative to the 5′ terminal nucleotide, of the guide strand and the internucleotidic linkage to the penultimate 3′ (N−1) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N−1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N−1) nucleotide and the 3′ terminal (N) nucleotide.

In certain embodiments, the guide strand comprises backbone phosphorothioate chiral centers in Sp configuration between the 3′ terminal nucleotide and the penultimate (N−1) nucleotide and as between the penultimate (N−1) nucleotide and the immediately upstream (N−2) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N−1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone phosphorothioate chiral centers in Rp or Sp configuration. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N−1) nucleotide and the 3′ terminal (N) nucleotide.

In certain embodiments, the guide strand comprises backbone phosphorothioate chiral centers in Rp, Sp, or alternating configurations between the 5′ terminal (+1) nucleotide and the immediately downstream (+2) nucleotide and between the +2 nucleotide and the immediately downstream (+3) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N−1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone chiral centers in Rp or Sp configuration. In certain embodiments, the one or more Rp, Sp, or stereorandomnon-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N−1) nucleotide and the 3′ terminal (N) nucleotide.

In certain embodiments, the guide strand comprises one or more backbone phosphorothioate chiral centers in Rp or Sp configuration upstream of backbone chiral centers in Sp configuration between the 3′ terminal nucleotide and the penultimate (N−1) nucleotide and as between the penultimate (N−1) nucleotide and the immediately upstream (N−2) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N−1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone chiral centers in Rp or Sp configuration. In certain embodiments, the one or more Rp, Sp, or stereorandomnon-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N−1) nucleotide and the 3′ terminal (N) nucleotide.

In certain embodiments, the guide strand comprises one or more backbone phosphorothioate chiral centers in Rp or Sp configuration between the 5′ terminal (+1) nucleotide and the immediately downstream (+2) nucleotide and between the (+2) nucleotide and the immediately downstream (+3) nucleotide, as well as between one or both of (a) the (+3) nucleotide and the (+4) nucleotide; and (b) the (+5) nucleotide and the (+6) nucleotide, and one or more of
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N−1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone chiral centers in Rp or Sp configuration. In certain embodiments, the one or more Rp, Sp, or stereorandomnon-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N−1) nucleotide and the 3′ terminal (N) nucleotide.

In certain embodiments, the guide strand comprises one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N−1) nucleotide of the guide strand, where N is the 3′ terminal nucleotide, a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone chiral centers in Rp or Sp configuration. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N−1) nucleotide and the 3′ terminal (N) nucleotide.

In certain embodiments, the guide strand comprises backbone phosphorothioate chiral centers in Sp configuration between the 3′ terminal nucleotide and the penultimate (N−1) nucleotide and as between the penultimate (N−1) nucleotide and the immediately upstream (N−2) nucleotide, a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49 and one or more backbone chiral centers in Rp or Sp configuration. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N−1) nucleotide and the 3′ terminal (N) nucleotide.

In certain embodiments, the guide strand comprises backbone phosphorothioate chiral centers in Rp, Sp, or alternating configurations between the 5′ terminal (+1) nucleotide and the immediately downstream (+2) nucleotide and between the +2 nucleotide and the immediately downstream (+3) nucleotide, a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49 and one or more backbone chiral centers in Rp or Sp configuration. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N−1) nucleotide and the 3′ terminal (N) nucleotide.

In certain embodiments, the guide strand comprises one or more backbone phosphorothioate chiral centers in Rp or Sp configuration upstream of backbone phosphorothioate chiral centers in Sp configuration between the 3′ terminal nucleotide and the penultimate (N−1) nucleotide and as between the penultimate (N−1) nucleotide and the immediately upstream (N−2) nucleotide, a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49 and one or more backbone chiral centers in Rp or Sp configuration. In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N−1) nucleotide and the 3′ terminal (N) nucleotide.

In certain embodiments, the guide strand comprises one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N−1) nucleotide of the guide strand, where N is the 3′ terminal nucleotide, a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49 and one or more backbone chiral centers in Rp or Sp configuration. In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain embodiments, the passenger strand comprises an Sp backbone phosphorothioate chiral center between the 5′ terminal (+1) nucleotide and the immediately downstream (+2) nucleotide and an Sp backbone phosphorothioate chiral center between the penultimate (N−1) nucleotide and the 3′ terminal (N) nucleotide.

In certain embodiments, a RNAi oligonucleotide comprises a sequence that is completely or substantially identical to or is completely or substantially complementary to 10 or more (e.g., 10, 11, 12, 13, 14, 15, 16, 17, 18, 19, 20 or more) contiguous bases of a target genomic sequence or a transcript therefrom (e.g., mRNA (e.g., pre-mRNA, mRNA after splicing, etc.)). In certain embodiments, a RNAi oligonucleotide comprises a sequence that is completely complementary to 10 or more (e.g., 10, 11, 12, 13, 14, 15, 16, 17, 18, 19, 20 or more) contiguous bases of a target transcript. In certain embodiments, the number of contiguous bases is about 15-20. In certain embodiments, the number of contiguous bases is about 20. In certain embodiments, an RNAi oligonucleotide that can hybridize with a target transcript (e.g., pre-mRNA, RNA, etc.) and can reduce the level of the target transcript and/or a protein encoded by the target transcript.

In certain embodiments, the present disclosure provides a dsRNAi oligonucleotide as disclosed herein, e.g., in Table 1. In certain embodiments, the present disclosure provides a dsRNAi oligonucleotide having a base sequence disclosed herein, e.g., in Table 1, or a portion thereof comprising at least 10 (e.g., 10, 11, 12, 13, 14, 15, 16, 17, 18, 19, 20 or more) contiguous bases, wherein the RNAi oligonucleotide is stereorandom or not chirally controlled, and wherein each T can be independently substituted with U and vice versa.

In certain embodiments, internucleotidic linkages of an oligonucleotide comprise or consist of 1-5, 1-10, 1-15, 1-20, 1-25, 1-30, 1-40, 1-50, or 1, 2, 3, 4, 5, 6, 7, 8, 9, 10, 11, 12, 13, 14, 15, 16, 17, 18, 19, 20 or more chirally controlled internucleotidic linkages. In certain embodiments, the present disclosure provides a dsRNAi oligonucleotide composition wherein the dsRNAi oligonucleotides comprise at least one chirally controlled internucleotidic linkage. In certain embodiments, the present disclosure provides a dsRNAi oligonucleotide composition wherein the dsRNAi oligonucleotides are stereorandom or not chirally controlled. In certain embodiments, in a dsRNAi oligonucleotide, at least one internucleotidic linkage is stereorandom and at least one internucleotidic linkage is chirally controlled.

In certain embodiments, internucleotidic linkages of an oligonucleotide comprise or consist of one or more neutrally charged internucleotidic linkages.

### 1.1 Double Stranded Oligonucleotides

In certain embodiments, the present disclosure provides oligonucleotides of various designs, which may comprise various nucleobases and patterns thereof, sugars and patterns thereof, internucleotidic linkages and patterns thereof, and/or additional chemical moieties and patterns thereof as described in the present disclosure. In certain embodiments, provided dsRNAi oligonucleotides can direct a decrease in the expression, level and/or activity of a gene and/or one or more of its products (e.g., transcripts, mRNA, proteins, etc.). In certain embodiments, provided dsRNAi oligonucleotides can direct a decrease in the expression, level and/or activity of a gene and/or one or more of its products in a cell of a subject or patient. In certain embodiments, a cell normally expresses or produces a protein. In certain embodiments, provided dsRNAi oligonucleotides can direct a decrease in the expression, level and/or activity of a target gene or a gene product and has a base sequence which consists of, comprises, or comprises a portion (e.g., a span of 1-5, 1-10, 1-15, 1-20, 1-25, 1-30, 1-40, 1-50, or 1,2,3, 4, 5, 6, 7, 8, 9, 10, 11, 12, 13, 14, 15, 16, 17, 18, 19, 20 or more contiguous bases) of the base sequence of a dsRNAi oligonucleotide disclosed herein, wherein each T can be independently substituted with U and vice versa, and the ds oligonucleotide comprises at least one non-naturally-occurring modification of a base, sugar and/or internucleotidic linkage.

In certain embodiments, dsRNAi oligonucleotides can direct a decrease in the expression, level and/or activity of a target gene, e.g., a target gene, or a product thereof. In certain embodiments, provided ds oligonucleotides can direct a decrease in the expression and/or level of a target gene or its gene product. In certain embodiments, provided ds oligonucleotides can direct a decrease in levels of target products. In certain embodiments, provided ds oligonucleotide can reduce levels of transcripts of target genes. In certain embodiments, provided ds oligonucleotide can reduce levels of mRNA of target genes. In certain embodiments, provided ds oligonucleotide can reduce levels of proteins encoded by target genes. In certain embodiments, provided ds oligonucleotides can direct a decrease in the expression and/or level of a target gene or its gene product via RNA interference. In certain embodiments, provided ds oligonucleotides can direct a decrease in the expression and/or level of a target gene or its gene product via a biochemical mechanism which does not involve RNA interference or RISC (including, but not limited to, RNaseH-mediated knockdown or steric hindrance of gene expression). In certain embodiments, provided ds oligonucleotides can direct a decrease in the expression and/or level of a target gene or its gene product via RNA interference and/or RNase H-mediated knockdown. In certain embodiments, provided ds oligonucleotides can direct a decrease in the expression and/or level of a target gene or its gene product by sterically blocking translation after binding to a target gene mRNA, and/or by altering or interfering with mRNA splicing and/or exon inclusion or exclusion. In certain embodiments, provided ds oligonucleotides comprise one or more structural elements described herein or known in the art in accordance with the present disclosure, e.g., base sequences; modifications; stereochemistry; patterns of internucleotidic linkages; GC contents; long GC stretches; patterns of backbone linkages; patterns of backbone chiral centers; patterns of backbone phosphorus modifications; additional chemical moieties, including but not limited to, one or more targeting moieties, lipid moieties, and/or carbohydrate moieties, etc.; seed regions; post-seed regions; 5′-end structures; 5′-end regions; 5′ nucleotide moieties; 3′-end regions; 3′-terminal dinucleotides; 3′-end caps; etc. In certain embodiments, a seed region of an oligonucleotide is or comprises the second to eighth, second to seventh, second to sixth, third to eighth, third to seventh, third to seven, or fourth to eighth or fourth to seventh nucleotides, counting from the 5′ end; and the post-seed region of the oligonucleotide is the region immediately 3′ to the seed region, and interposed between the seed region and the 3′ end region. In certain embodiments, a provided composition comprises a ds oligonucleotide. In certain embodiments, a provided composition comprises one or more lipid moieties, one or more carbohydrate moieties (unless otherwise specified, other than sugar moieties of nucleoside units that form oligonucleotide chain with internucleotidic linkages), and/or one or more targeting components. In certain embodiments, ds RNAi oligonucleotides can direct a decrease in the expression, level and/or activity of a target gene or a product thereof by sterically blocking translation after binding to a target gene mRNA, and/or by altering or interfering with mRNA splicing. Regardless, however, the present disclosure is not limited to any particular mechanism. In certain embodiments, the present disclosure provides ds oligonucleotides, compositions, methods, etc., capable of operating via double-stranded RNA interference, single-stranded RNA interference, RNase H-mediated knock-down, steric hindrance of translation, or a combination of two or more such mechanisms.

In certain embodiments, a dsRNAi oligonucleotide comprises a structural element or a portion thereof described herein, e.g., in Table 1. In certain embodiments, a dsRNAi oligonucleotide comprises a base sequence (or a portion thereof) described herein, wherein each T can be independently substituted with U and vice versa, a chemical modification or a pattern of chemical modifications (or a portion thereof), and/or a format or a portion thereof described herein. In certain embodiments, a dsRNAi oligonucleotide has a base sequence which comprises the base sequence (or a portion thereof) wherein each T can be independently substituted with U, pattern of chemical modifications (or a portion thereof), and/or a format of an oligonucleotide disclosed herein, e.g., in Table 1, or otherwise disclosed herein. In certain embodiments, such ds oligonucleotides, e.g., dsRNAi oligonucleotides reduce expression, level and/or activity of a gene, e.g., a gene, or a gene product thereof.

Among other things, dsRNAi oligonucleotides may hybridize to their target nucleic acids (e.g., pre-mRNA, mature mRNA, etc.). For example, in certain embodiments, a dsRNAi oligonucleotide can hybridize to a nucleic acid derived from a DNA strand (either strand of the gene). In certain embodiments, a dsRNAi oligonucleotide can hybridize to a transcript. In certain embodiments, a dsRNAi oligonucleotide can hybridize to a target nucleic acid in any stage of RNA processing, including but not limited to a pre-mRNA or a mature mRNA. In certain embodiments, a dsRNAi oligonucleotide can hybridize to any element of a target nucleic acid or its complement, including but not limited to: a promoter region, an enhancer region, a transcriptional stop region, a translational start signal, a translation stop signal, a coding region, a non-coding region, an exon, an intron, an intron/exon or exon/intron junction, the 5′ UTR, or the 3′ UTR. In certain embodiments, dsRNAi oligonucleotides can hybridize to their targets with no more than 2 mismatches. In certain embodiments, dsRNAi oligonucleotides can hybridize to their targets with no more than one mismatch. In certain embodiments, dsRNAi oligonucleotides can hybridize to their targets with no mismatches (e.g., when all C-G and/or A-T/U base paring).

In certain embodiments, a ds oligonucleotide can hybridize to two or more variants of transcripts. In certain embodiments, a dsRNAi oligonucleotide can hybridize to two or more or all variants of a transcript. In certain embodiments, a dsRNAi oligonucleotide can hybridize to two or more or all variants of a transcript derived from the sense strand.

In certain embodiments, a target of a dsRNAi oligonucleotide is a RNA which is not a mRNA.

In certain embodiments, ds oligonucleotides, e.g., dsRNAi oligonucleotides, contain increased levels of one or more isotopes. In certain embodiments, ds oligonucleotides, e.g., dsRNAi oligonucleotides, are labeled, e.g., by one or more isotopes of one or more elements, e.g., hydrogen, carbon, nitrogen, etc. In certain embodiments, ds oligonucleotides, e.g., dsRNAi oligonucleotides, in provided compositions, e.g., ds oligonucleotides of a plurality of a composition, comprise base modifications, sugar modifications, and/or internucleotidic linkage modifications, wherein the ds oligonucleotides contain an enriched level of deuterium. In certain embodiments, oligonucleotides, e.g., RNAi oligonucleotides, are labeled with deuterium (replacing -¹H with -²H) at one or more positions. In certain embodiments, one or more ¹H of a ds oligonucleotide chain or any moiety conjugated to the ds oligonucleotide chain (e.g., a targeting moiety, etc.) is substituted with ²H. Such ds oligonucleotides can be used in compositions and methods described herein.

In certain embodiments, the present disclosure provides a ds oligonucleotide composition comprising a plurality of ds oligonucleotides which:
1) have a common base sequence complementary to a target sequence (e.g., a target sequence) in a transcript; and
2) comprise one or more modified sugar moieties and/or modified internucleotidic linkages.

In certain embodiments, dsRNAi oligonucleotides having a common base sequence may have the same pattern of nucleoside modifications, e.g., sugar modifications, base modifications, etc. In certain embodiments, a pattern of nucleoside modifications may be represented by a combination of locations and modifications. In certain embodiments, a pattern of backbone linkages comprises locations and types (e.g., phosphate, phosphorothioate, substituted phosphorothioate, etc.) of each internucleotidic linkage.

In certain embodiments, ds oligonucleotides of a plurality, e.g., in provided compositions, are of the same ds oligonucleotide type. In certain embodiments, ds oligonucleotides of an ds oligonucleotide type have a common pattern of sugar modifications. In certain embodiments, ds oligonucleotides of a ds oligonucleotide type have a common pattern of base modifications. In certain embodiments, ds oligonucleotides of a ds oligonucleotide type have a common pattern of nucleoside modifications. In certain embodiments, ds oligonucleotides of a ds oligonucleotide type have the same constitution. In certain embodiments, ds oligonucleotides of a ds oligonucleotide type are identical. In certain embodiments, ds oligonucleotides of a plurality are identical. In certain embodiments, ds oligonucleotides of a plurality share the same constitution.

In certain embodiments, as exemplified herein, dsRNAi oligonucleotides are chiral controlled, comprising one or more chirally controlled internucleotidic linkages. In certain embodiments, ds RNAi oligonucleotides are stereochemically pure. In certain embodiments, dsRNAi oligonucleotides are substantially separated from other stereoisomers.

In certain embodiments, RNAi oligonucleotides comprise one or more modified nucleobases, one or more modified sugars, and/or one or more modified internucleotidic linkages.

In certain embodiments, dsRNAi oligonucleotides comprise one or more modified sugars. In certain embodiments, ds oligonucleotides of the present disclosure comprise one or more modified nucleobases. Various modifications can be introduced to a sugar and/or nucleobase in accordance with the present disclosure. For example, in certain embodiments, a modification is a modification described in U.S. Pat. No. 9,006,198. In certain embodiments, a modification is a modification described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612, the sugar, base, and internucleotidic linkage modifications of each of which are independently incorporated herein by reference.

As used in the present disclosure, in certain embodiments, “one or more” is 1-200, 1-150, 1-100, 1-90, 1-80, 1-70, 1-60, 1-50, 1-40, 1-30, or 1, 2, 3, 4, 5, 6, 7, 8, 9, 10, 11, 12, 13, 14, 15, 16, 17, 18, 19, 20, 21, 22, 23, 24, or 25. In certain embodiments, “one or more” is one. In certain embodiments, “one or more” is two. In certain embodiments, “one or more” is three. In certain embodiments, “one or more” is four. In certain embodiments, “one or more” is five. In certain embodiments, “one or more” is six. In certain embodiments, “one or more” is seven. In certain embodiments, “one or more” is eight. In certain embodiments, “one or more” is nine. In certain embodiments, “one or more” is ten. In certain embodiments, “one or more” is at least one. In certain embodiments, “one or more” is at least two. In certain embodiments, “one or more” is at least three. In certain embodiments, “one or more” is at least four. In certain embodiments, “one or more” is at least five. In certain embodiments, “one or more” is at least six. In certain embodiments, “one or more” is at least seven. In certain embodiments, “one or more” is at least eight. In certain embodiments, “one or more” is at least nine. In certain embodiments, “one or more” is at least ten.

As used in the present disclosure, in certain embodiments, “at least one” is 1-200, 1-150, 1-100, 1-90, 1-80, 1-70, 1-60, 1-50, 1-40, 1-30, or 1, 2, 3, 4, 5, 6, 7, 8, 9, 10, 11, 12, 13, 14, 15, 16, 17, 18, 19, 20, 21, 22, 23, 24, or 25. In certain embodiments, “at least one” is one. In certain embodiments, “at least one” is two. In certain embodiments, “at least one” is three. In certain embodiments, “at least one” is four. In certain embodiments, “at least one” is five. In certain embodiments, “at least one” is six. In certain embodiments, “at least one” is seven. In certain embodiments, “at least one” is eight. In certain embodiments, “at least one” is nine. In certain embodiments, “at least one” is ten.

In certain embodiments, a dsRNAi oligonucleotide is or comprises a dsRNAi oligonucleotide described in Table 1.

As demonstrated in the present disclosure, in certain embodiments, a provided ds oligonucleotide (e.g., a dsRNAi oligonucleotide) is characterized in that, when it is contacted with the transcript in a knockdown system, knockdown of its target (e.g., a transcript for a target oligonucleotide).

In certain embodiments, ds oligonucleotides are provided as salt forms. In certain embodiments, ds oligonucleotides are provided as salts comprising negatively-charged internucleotidic linkages (e.g., phosphorothioate internucleotidic linkages, natural phosphate linkages, etc.) existing as their salt forms. In certain embodiments, ds oligonucleotides are provided as pharmaceutically acceptable salts. In certain embodiments, ds oligonucleotides are provided as metal salts. In certain embodiments, ds oligonucleotides are provided as sodium salts. In certain embodiments, ds oligonucleotides are provided as metal salts, e.g., sodium salts, wherein each negatively-charged internucleotidic linkage is independently in a salt form (e.g., for sodium salts, —O—P(O)(SNa)—O— for a phosphorothioate internucleotidic linkage, —O—P(O)(ONa)—O— for a natural phosphate linkage, etc.).

### 1.2 Regions of Double Stranded Oligonucleotides

### 1.2.1 Base Sequences

In certain embodiments, a dsRNAi oligonucleotide comprises a base sequence described herein or a portion (e.g., a span of 5-50, 5-40, 5-30, 5-20, or 5, 6, 7, 8, 9, 10, 11, 12, 13, 14, 15, 16, 17, 18, 19, 20, 21, 22, 23, 24, 25, 26, 27, 28, 20 or at least 10, at least 15, contiguous nucleobases) thereof with 0-5 (e.g., 0, 1, 2, 3, 4 or 5) mismatches, wherein each T can be independently substituted with U and vice versa. In certain embodiments, a dsRNAi oligonucleotide comprises a base sequence described herein, or a portion thereof, wherein a portion is a span of at least 10 contiguous nucleobases, or a span of at least 15 contiguous nucleobases with 1-5 mismatches. In certain embodiments, dsRNAi oligonucleotides comprise a base sequence described herein, or a portion thereof, wherein a portion is a span of at least 10 contiguous nucleobases, or a span of at least 10 contiguous nucleobases with 1-5 mismatches, wherein each T can be independently substituted with U and vice versa. In certain embodiments, base sequences of ds oligonucleotides comprise or consists of 10-50 (e.g., about or at least 10, 11, 12, 13, 14, 15, 16, 17, 18, 19, 20, 21, 22, 23, 24, 25, 30, 35, 40, 45; in certain embodiments, at least 15; in certain embodiments, at least 16; in certain embodiments, at least 17; in certain embodiments, at least 18; in certain embodiments, at least 19; in certain embodiments, at least 20; in certain embodiments, at least 21; in certain embodiments, at least 22; in certain embodiments, at least 23; in certain embodiments, at least 24; in certain embodiments, at least 25) contiguous bases of a base sequence that is identical to or complementary to a base sequence of a gene or a transcript (e.g., mRNA) thereof.

Base sequences of the guide strand of dsRNAi oligonucleotides, as appreciated by those skilled in the art, typically have sufficient length and complementarity to their targets, e.g., RNA transcripts (e.g., pre-mRNA, mature mRNA, etc.) to mediate target-specific knockdown. In certain embodiments, the base sequence of a dsRNAi oligonucleotide guide strand has a sufficient length and identity to a transcript target to mediate target-specific knockdown. In certain embodiments, the dsRNAi oligonucleotide guide strand is complementary to a portion of a transcript (a transcript target sequence). In certain embodiments, the base sequence of a dsRNAi oligonucleotide has 90% or more identity with the base sequence of a ds oligonucleotide disclosed in Table 1, wherein each T can be independently substituted with U and vice versa. In certain embodiments, the base sequence of a dsRNAi oligonucleotide has 95% or more identity with the base sequence of an oligonucleotide disclosed in Table 1, wherein each T can be independently substituted with U and vice versa. In certain embodiments, the base sequence of a dsRNAi oligonucleotide comprises a continuous span of 15 or more bases of an oligonucleotide disclosed in Table 1, wherein each T can be independently substituted with U and vice versa, except that one or more bases within the span are abasic (e.g., a nucleobase is absent from a nucleotide). In certain embodiments, the base sequence of a dsRNAi oligonucleotide comprises a continuous span of 19 or more bases of a dsRNAi oligonucleotide disclosed herein, except that one or more bases within the span are abasic (e.g., a nucleobase is absent from a nucleotide). In certain embodiments, the base sequence of a dsRNAi oligonucleotide comprises a continuous span of 19 or more bases of a ds oligonucleotide disclosed herein, wherein each T can be independently substituted with U and vice versa, except for a difference in the 1 or 2 bases at the 5′ end and/or 3′ end of the base sequences.

In certain embodiments, the present disclosure pertains to a ds oligonucleotide having a base sequence which comprises the base sequence of any ds oligonucleotide disclosed herein, wherein each T may be independently replaced with U and vice versa.

In certain embodiments, the present disclosure pertains to a ds oligonucleotide having a base sequence which comprises at least 15 contiguous bases of the base sequence of any ds oligonucleotide disclosed herein, wherein each T may be independently replaced with U and vice versa.

In certain embodiments, the present disclosure pertains to a ds oligonucleotide having a base sequence which is at least 90% identical to the base sequence of any ds oligonucleotide disclosed herein, wherein each T may be independently replaced with U and vice versa.

In certain embodiments, the present disclosure pertains to a ds oligonucleotide having a base sequence which is at least 95% identical to the base sequence of any ds oligonucleotide disclosed herein, wherein each T may be independently replaced with U and vice versa.

In certain embodiments, a base sequence of a ds oligonucleotide is, comprises, or comprises 10-20, e.g., 10, 11, 12, 13, 14, 15, 16, 17, 18, 19 or 20 contiguous bases of the base sequence of any ds oligonucleotide described herein, wherein each T may be independently replaced with U and vice versa.

In certain embodiments, a dsRNAi oligonucleotide is selected from Table 1.

In certain embodiments, a dsRNAi oligonucleotide target two or more or all alleles (if multiple alleles exist in a relevant system). In certain embodiments, a ds oligonucleotide reduces expressions, levels and/or activities of both wild-type allele and mutant allele, and/or transcripts and/or products thereof.

In certain embodiments, base sequences of provided ds oligonucleotides are fully complementary to both human and a non-human primate (NHP) target sequences. In certain embodiments, such sequences can be particularly useful as they can be readily assessed in both human and non-human primates.

In certain embodiments, a dsRNAi oligonucleotide comprises a base sequence or portion thereof described in Table 1, wherein each T may be independently replaced with U and vice versa, and/or a sugar, nucleobase, and/or internucleotidic linkage modification and/or a pattern thereof described in Table 1, and/or an additional chemical moiety (in addition to an oligonucleotide chain, e.g., a target moiety, a lipid moiety, a carbohydrate moiety, etc.) described in Table 1.

In certain embodiments, the terms “complementary,” “fully complementary” and “substantially complementary” may be used with respect to the base matching between n ds oligonucleotide (e.g., a dsRNAi oligonucleotide) base sequence and a target sequence, as will be understood by those skilled in the art from the context of their use. It is noted that substitution of T for U, or vice versa, generally does not alter the amount of complementarity. As used herein, a ds oligonucleotide that is “substantially complementary” to a target sequence is largely or mostly complementary but not 100% complementary. In certain embodiments, a sequence (e.g., a dsRNAi oligonucleotide) which is substantially complementary has 1, 2, 3, 4 or 5 mismatches when aligned to its target sequence. In certain embodiments, a dsRNAi oligonucleotide has a base sequence which is substantially complementary to ai target sequence. In certain embodiments, a dsRNAi oligonucleotide has a base sequence which is substantially complementary to the complement of the sequence of a dsRNAi oligonucleotide disclosed herein. As appreciated by those skilled in the art, in certain embodiments, sequences of ds oligonucleotides need not be 100% complementary to their targets for the ds oligonucleotides to perform their functions (e.g., knockdown of target nucleic acids. Typically when determining complementarity, A and T (or U) are complementary nucleobases and C and G are complementary nucleobases.

In certain embodiments, a “portion” (e.g., of a base sequence or a pattern of modifications) is at least 5, 6, 7, 8, 9, 10, 11, 12, 13, 14, 15, 16, 17, 18, 19, or 20 monomeric units long (e.g., for a base sequence, at least 5, 6, 7, 8, 9, 10, 11, 12, 13, 14, 15, 16, 17, 18, 19, or 20 bases long). In certain embodiments, a “portion” of a base sequence is at least 5 bases long. In certain embodiments, a “portion” of a base sequence is at least 10 bases long. In certain embodiments, a “portion” of a base sequence is at least 15 bases long. In certain embodiments, a “portion” of a base sequence is at least 16, 17, 18, 19 or 20 bases long. In certain embodiments, a “portion” of a base sequence is at least 20 bases long. In certain embodiments, a portion of a base sequence is 10, 11, 12, 13, 14, 15, 16, 17, 18, 19 or more contiguous (consecutive) bases. In certain embodiments, a portion of a base sequence is 15 or more contiguous (consecutive) bases. In certain embodiments, a portion of a base sequence is 16, 17, 18, 19 or 20 or more contiguous (consecutive) bases. In certain embodiments, a portion of a base sequence is 20 or more contiguous (consecutive) bases.

In certain embodiments, a portion is a span of at least 15, 16, 17, 18, 19, 20, 21, 22, 23, 24, or 25 total nucleotides. In certain embodiments, a portion is a span of at least 15, 16, 17, 18, 19, 20, 21, 22, 23, 24, or 25 total nucleotides with 0-3 mismatches. In certain embodiments, a portion is a span of at least 15, 16, 17, 18, 19, 20, 21, 22, 23, 24, or 25 total nucleotides with 0-3 mismatches, wherein a span with 0 mismatches is complementary and a span with 1 or more mismatches is anon-limiting example of substantial complementarity. In certain embodiments, a base comprises a portion characteristic of a nucleic acid (e.g., a gene) in that the portion is identical or complementary to a portion of the nucleic acid or a transcript thereof, and is not identical or complementary to a portion of any other nucleic acid (e.g., a gene) or a transcript thereof in the same genome. In certain embodiments, a portion is characteristic of human dsRNAi.

In certain embodiments, a provided oligonucleotide, e.g., a dsRNAi oligonucleotide, has a length of no more than about 49, 45, 40, 30, 35, 25, or 23 total nucleotides as described herein. In certain embodiments, wherein the sequence recited herein starts with a U or T at the 5′-end, the U can be deleted and/or replaced by another base.

In certain embodiments, ds oligonucleotides, e.g., dsRNAi oligonucleotides are stereorandom. In certain embodiments, RNAi oligonucleotides are chirally controlled. In certain embodiments, a ds RNAi oligonucleotide is chirally pure (or “stereopure”, “stereochemically pure”), wherein the ds oligonucleotide exists as a single stereoisomeric form (in many cases a single diastereoisomeric (or “diastereomeric”) form as multiple chiral centers may exist in a ds oligonucleotide, e.g., at linkage phosphorus, sugar carbon, etc.). As appreciated by those skilled in the art, a chirally pure ds oligonucleotide is separated from its other stereoisomeric forms (to the extent that some impurities may exist as chemical and biological processes, selectivities and/or purifications etc. rarely, if ever, go to absolute completeness). In a chirally pure ds oligonucleotide, each chiral center is independently defined with respect to its configuration (for a chirally pure ds oligonucleotide, each internucleotidic linkage is independently stereodefined or chirally controlled). In contrast to chirally controlled and chirally pure ds oligonucleotides which comprise stereodefined linkage phosphorus, racemic (or “stereorandom”, “non-chirally controlled”) ds oligonucleotides comprising chiral linkage phosphorus, e.g., from traditional phosphoramidite oligonucleotide synthesis without stereochemical control during coupling steps in combination with traditional sulfurization (creating stereorandom phosphorothioate internucleotidic linkages), refer to a random mixture of various stereoisomers (typically diastereoisomers (or “diastereomers”) as there are multiple chiral centers in a ds oligonucleotide; e.g., from traditional ds oligonucleotide preparation using reagents containing no chiral elements other than those in nucleosides and linkage phosphorus). For example, for A*A*A wherein * is a phosphorothioate internucleotidic linkage (which comprises a chiral linkage phosphorus), a racemic oligonucleotide preparation includes four diastereomers [2²=4, considering the two chiral linkage phosphorus, each of which can exist in either of two configurations (Sp or Rp)]: A *S A *S A, A *S A *R A, A *R A *S A, and A *R A *R A, wherein *S represents a Sp phosphorothioate internucleotidic linkage and *R represents a Rp phosphorothioate internucleotidic linkage. For a chirally pure oligonucleotide, e.g., A *S A *S A, it exists in a single stereoisomeric form and it is separated from the other stereoisomers (e.g., the diastereomers A *S A *R A, A *R A *S A, and A *RA *RA).

In certain embodiments, dsRNAi oligonucleotides comprise 1, 2, 3, 4, 5, 6, 7, 8, 9, 10 or more stereorandom internucleotidic linkages (mixture of Rp and Sp linkage phosphorus at the internucleotidic linkage, e.g., from traditional non-chirally controlled oligonucleotide synthesis). In certain embodiments, dsRNAi oligonucleotides comprise one or more (e.g., 1-50, 1-40, 1-30, 1-25, 1-20, 1, 2, 3, 4, 5, 6, 7, 8, 9, 10, 11, 12, 13, 14, 15, 16, 17, 18, 19, 20, 21, 22, 23, 24, 25 or more) chirally controlled internucleotidic linkages (Rp or Sp linkage phosphorus at the internucleotidic linkage, e.g., from chirally controlled oligonucleotide synthesis).

In certain embodiments, an internucleotidic linkage is a phosphorothioate internucleotidic linkage. In certain embodiments, an internucleotidic linkage is a stereorandom phosphorothioate internucleotidic linkage. In certain embodiments, an internucleotidic linkage is a chirally controlled phosphorothioate internucleotidic linkage.

Among other things, the present disclosure provides technologies for preparing chirally controlled (in certain embodiments, stereochemically pure) ds oligonucleotides. In certain embodiments, ds oligonucleotides are stereochemically pure. In certain embodiments, ds oligonucleotides of the present disclosure are about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 15%, 20%, 25%, 30%, 35%, 40%, 45%, 50%, 55%, 60%, 65%, 70%, 75%, 80%, 85%, 90%, 95%, or 100%, or at least about 5%, 10%, 15%, 20%, 25%, 30%, 35%, 40%, 45%, 50%, 55%, 60%, 65%, 70%, 75%, 80%, 85%, 90%, 95%, or 99%, pure. In certain embodiments, internucleotidic linkages of ds oligonucleotides comprise or consist of one or more (e.g., 1-50, 1-40, 1-30, 1-25, 1-20, 5-50, 5-40, 5-30, 5-25, 5-20, 1, 2, 3, 4, 5, 6, 7, 8, 9, 10, 11, 12, 13, 14, 15, 16, 17, 18, 19, 20, 21, 22, 23, 24, 25 or more) chiral internucleotidic linkages, each of which independently has a diastereopurity of at least 80%, 85%, 90%, 91%, 92%, 93%, 94%, 95%, 96%, 97%, 98%, 99% or 99.5%, typically at least 90%, 91%, 92%, 93%, 94%, 95%, 96%, 97%, 98%, 99% or 99.5%. In certain embodiments, ds oligonucleotides of the present disclosure, e.g., dsRNAi oligonucleotides, have a diastereopurity of (DS)^{CIL}, wherein DS is a diastereopurity as described in the present disclosure (e.g., 90%, 91%, 92%, 93%, 94%, 95%, 96%, 97%, 98%, 99% or 99.5% or more) and CIL is the number of chirally controlled internucleotidic linkages (e.g., 1-50, 1-40, 1-30, 1-25, 1-20, 5-50, 5-40, 5-30, 5-25, 5-20, 1, 2, 3, 4, 5, 6, 7, 8, 9, 10, 11, 12, 13, 14, 15, 16, 17, 18, 19, 20, 21, 22, 23, 24, 25 or more). In certain embodiments, DS is 95%-100%. In certain embodiments, each internucleotidic linkage is independently chirally controlled, and CIL is the number of chirally controlled internucleotidic linkages.

As examples, certain dsRNAi oligonucleotides comprising certain example base sequences, nucleobase modifications and patterns thereof, sugar modifications and patterns thereof, internucleotidic linkages and patterns thereof, linkage phosphorus stereochemistry and patterns thereof, linkers, and/or additional chemical moieties are presented in Table 1, below. Among other things, ds oligonucleotides, e.g., those in Table 1A, may be utilized to target a transcript, e.g., to reduce the level of a transcript and/or a product thereof.

TABLE 1
Example Oligonucleotides/Compositions that target TTR.

| | | | Stereochemistry/ |
|---|---|---|---|
| ID | Description | Naked Sequence | linkage |
| WV-46497 | mUn001RfU*SmAmUmAfGmAmGmCmAmAmGmAfAmCfAmCmUm | UUAUAGAGCAAGAACACUGUU | nRSOOOOOOOOOOOOOOOO |
| | GmUmU*SmU*SmU | UU | OOSS |
| WV-46498 | mU*RfUn001RmAmUmAfGmAmGmCmAmAmGmAfAmCfAmCmUm | UUAUAGAGCAAGAACACUGUU | RnROOOOOOOOOOOOOOOO |
| | GmUmU*SmU*SmU | UU | OOSS |
| WV-46499 | mU*RfU*SmAn001RmUmAfGmAmGmCmAmAmGmAfAmCfAmCm | UUAUAGAGCAAGAACACUGUU | RSnROOOOOOOOOOOOOOO |
| | UmGmUmU*SmU*SmU | UU | OOSS |
| WV-46500 | mU*RfU*SmAmUn001RmAfGmAmGmCmAmAmGmAfAmCfAmCm | UUAUAGAGCAAGAACACUGUU | RSOnROOOOOOOOOOOOOO |
| | UmGmUmU*SmU*SmU | UU | OOSS |
| WV-46501 | mU*RfU*SmAmUmAn001RfGmAmGmCmAmAmGmAfAmCfAmCm | UUAUAGAGCAAGAACACUGUU | RSOOnROOOOOOOOOOOOO |
| | UmGmUmU*SmU*SmU | UU | OOSS |
| WV-46502 | mU*RfU*SmAmUmAfGn001RmAmGmCmAmAmGmAfAmCfAmCm | UUAUAGAGCAAGAACACUGUU | RSOOOnROOOOOOOOOOOO |
| | UmGmUmU*SmU*SmU | UU | OOSS |
| WV-46503 | mU*RfU*SmAmUmAfGmAn001RmGmCmAmAmGmAfAmCfAmCm | UUAUAGAGCAAGAACACUGUU | RSOOOOnROOOOOOOOOOO |
| | UmGmUmU*SmU*SmU | UU | OOSS |
| WV-46504 | mU*RfU*SmAmUmAfGmAmGn001RmCmAmAmGmAfAmCfAmCm | UUAUAGAGCAAGAACACUGUU | RSOOOOOnROOOOOOOOOO |
| | UmGmUmU*SmU*SmU | UU | OOSS |
| WV-46505 | mU*RfU*SmAmUmAfGmAmGmCn001RmAmAmGmAfAmCfAmCm | UUAUAGAGCAAGAACACUGUU | RSOOOOOOnROOOOOOOOO |
| | UmGmUmU*SmU*SmU | UU | OOSS |
| WV-46506 | mU*RfU*SmAmUmAfGmAmGmCmAn001RmAmGmAfAmCfAmCm | UUAUAGAGCAAGAACACUGUU | RSOOOOOOOnROOOOOOOO |
| | UmGmUmU*SmU*SmU | UU | OOSS |
| WV-46507 | mU*RfU*SmAmUmAfGmAmGmCmAmAn001RmGmAfAmCfAmCm | UUAUAGAGCAAGAACACUGUU | RSOOOOOOOOnROOOOOOO |
| | UmGmUmU*SmU*SmU | UU | OOSS |
| WV-46508 | mU*RfU*SmAmUmAfGmAmGmCmAmAmGn001RmAfAmCfAmCm | UUAUAGAGCAAGAACACUGUU | RSOOOOOOOOOnROOOOOO |
| | UmGmUmU*SmU*SmU | UU | OOSS |
| WV-46509 | mU*RfU*SmAmUmAfGmAmGmCmAmAmGmAn001RfAmCfAmCm | UUAUAGAGCAAGAACACUGUU | RSOOOOOOOOOOnROOOOO |
| | UmGmUmU*SmU*SmU | UU | OOSS |
| WV-46510 | mU*RfU*SmAmUmAfGmAmGmCmAmAmGmAfAn001RmCfAmCm | UUAUAGAGCAAGAACACUGUU | RSOOOOOOOOOOOnROOOO |
| | UmGmUmU*SmU*SmU | UU | OOSS |
| WV-46511 | mU*RfU*SmAmUmAfGmAmGmCmAmAmGmAfAmCn001RfAmCm | UUAUAGAGCAAGAACACUGUU | RSOOOOOOOOOOOOnROOO |
| | UmGmUmU*SmU*SmU | UU | OOSS |
| WV-46512 | mU*RfU*SmAmUmAfGmAmGmCmAmAmGmAfAmCfAn001RmCm | UUAUAGAGCAAGAACACUGUU | RSOOOOOOOOOOOOOnROO |
| | UmGmUmU*SmU*SmU | UU | OOSS |
| WV-46513 | mU*RfU*SmAmUmAfGmAmGmCmAmAmGmAfAmCfAmCn001Rm | UUAUAGAGCAAGAACACUGUU | RSOOOOOOOOOOOOOOnRO |
| | UmGmUmU*SmU*SmU | UU | OOSS |
| WV-46514 | mU*RfU*SmAmUmAfGmAmGmCmAmAmGmAfAmCfAmCmUn001 | UUAUAGAGCAAGAACACUGUU | RSOOOOOOOOOOOOOOOnR |
| | RmGmUmU*SmU*SmU | UU | OOSS |
| WV-46515 | mU*RfU*SmAmUmAfGmAmGmCmAmAmGmAfAmCfAmCmUmGn0 | UUAUAGAGCAAGAACACUGUU | RSOOOOOOOOOOOOOOOOn |
| | 01RmUmU*SmU*SmU | UU | ROSS |
| WV-46516 | mU*RfU*SmAmUmAfGmAmGmCmAmAmGmAfAmCfAmCmUmGmU | UUAUAGAGCAAGAACACUGUU | RSOOOOOOOOOOOOOOOOO |
| | n001RmU*SmU*SmU | UU | nRSS |
| WV-46517 | mU*RfU*SmAmUmAfGmAmGmCmAmAmGmAfAmCfAmCmUmGmU | UUAUAGAGCAAGAACACUGUU | RSOOOOOOOOOOOOOOOOO |
| | mUn001RmU*SmU | UU | OnRS |
| WV-46518 | mU*RfU*SmAmUmAfGmAmGmCmAmAmGmAfAmCfAmCmUmGmU | UUAUAGAGCAAGAACACUGUU | RSOOOOOOOOOOOOOOOOO |
| | mU*SmUn001RmU | UU | OSnR |
| WV-46519 | mUn001SfU*SmAmUmAfGmAmGmCmAmAmGmAfAmCfAmCmUm | UUAUAGAGCAAGAACACUGUU | nSSOOOOOOOOOOOOOOOO |
| | GmUmU*SmU*SmU | UU | OOSS |
| WV-46520 | mU*RfUn001SmAmUmAfGmAmGmCmAmAmGmAfAmCfAmCmUm | UUAUAGAGCAAGAACACUGUU | RnSOOOOOOOOOOOOOOOO |
| | GmUmU*SmU*SmU | UU | OOSS |
| WV-45148 | mU*RfU*SmAn001SmUmAfGmAmGmCmAmAmGmAfAmCfAmCm | UUAUAGAGCAAGAACACUGUU | RSnSOOOOOOOOOOOOOOO |
| | UmGmUmU*SmU*SmU | UU | OOSS |
| WV-46521 | mU*RfU*SmAmUn001SmAfGmAmGmCmAmAmGmAfAmCfAmCm | UUAUAGAGCAAGAACACUGUU | RSOnSOOOOOOOOOOOOOO |
| | UmGmUmU*SmU*SmU | UU | OOSS |
| WV-46522 | mU*RfU*SmAmUmAn001SfGmAmGmCmAmAmGmAfAmCfAmCm | UUAUAGAGCAAGAACACUGUU | RSOOnSOOOOOOOOOOOOO |
| | UmGmUmU*SmU*SmU | UU | OOSS |
| WV-46523 | mU*RfU*SmAmUmAfGn001SmAmGmCmAmAmGmAfAmCfAmCm | UUAUAGAGCAAGAACACUGUU | RSOOOnSOOOOOOOOOOOO |
| | UmGmUmU*SmU*SmU | UU | OOSS |
| WV-46524 | mU*RfU*SmAmUmAfGmAn001SmGmCmAmAmGmAfAmCfAmCm | UUAUAGAGCAAGAACACUGUU | RSOOOOnSOOOOOOOOOOO |
| | UmGmUmU*SmU*SmU | UU | OOSS |
| WV-46525 | mU*RfU*SmAmUmAfGmAmGn001SmCmAmAmGmAfAmCfAmCm | UUAUAGAGCAAGAACACUGUU | RSOOOOOnSOOOOOOOOOO |
| | UmGmUmU*SmU*SmU | UU | OOSS |
| WV-46526 | mU*RfU*SmAmUmAfGmAmGmCn001SmAmAmGmAfAmCfAmCm | UUAUAGAGCAAGAACACUGUU | RSOOOOOOnSOOOOOOOOO |
| | UmGmUmU*SmU*SmU | UU | OOSS |
| WV-45147 | mU*RfU*SmAmUmAfGmAmGmCmAn001SmAmGmAfAmCfAmCm | UUAUAGAGCAAGAACACUGUU | RSOOOOOOOnSOOOOOOOO |
| | UmGmUmU*SmU*SmU | UU | OOSS |
| WV-46527 | mU*RfU*SmAmUmAfGmAmGmCmAmAn001SmGmAfAmCfAmCm | UUAUAGAGCAAGAACACUGUU | RSOOOOOOOOnSOOOOOOO |
| | UmGmUmU*SmU*SmU | UU | OOSS |
| WV-46528 | mU*RfU*SmAmUmAfGmAmGmCmAmAmGn001SmAfAmCfAmCm | UUAUAGAGCAAGAACACUGUU | RSOOOOOOOOOnSOOOOOO |
| | UmGmUmU*SmU*SmU | UU | OOSS |
| WV-46529 | mU*RfU*SmAmUmAfGmAmGmCmAmAmGmAn001SfAmCfAmCm | UUAUAGAGCAAGAACACUGUU | RSOOOOOOOOOOnSOOOOO |
| | UmGmUmU*SmU*SmU | UU | OOSS |
| WV-46530 | mU*RfU*SmAmUmAfGmAmGmCmAmAmGmAfAn001SmCfAmCm | UUAUAGAGCAAGAACACUGUU | RSOOOOOOOOOOOnSOOOO |
| | UmGmUmU*SmU*SmU | UU | OOSS |
| WV-46531 | mU*RfU*SmAmUmAfGmAmGmCmAmAmGmAfAmCn001SfAmCm | UUAUAGAGCAAGAACACUGUU | RSOOOOOOOOOOOOnSOOO |
| | UmGmUmU*SmU*SmU | UU | OOSS |
| WV-46532 | mU*RfU*SmAmUmAfGmAmGmCmAmAmGmAfAmCfAn001SmCm | UUAUAGAGCAAGAACACUGUU | RSOOOOOOOOOOOOOnSOO |
| | UmGmUmU*SmU*SmU | UU | OOSS |
| WV-46533 | mU*RfU*SmAmUmAfGmAmGmCmAmAmGmAfAmCfAmCn001Sm | UUAUAGAGCAAGAACACUGUU | RSOOOOOOOOOOOOOOnSO |
| | UmGmUmU*SmU*SmU | UU | OOSS |
| WV-46534 | mU*RfU*SmAmUmAfGmAmGmCmAmAmGmAfAmCfAmCmUn001 | UUAUAGAGCAAGAACACUGUU | RSOOOOOOOOOOOOOOOnS |
| | SmGmUmU*SmU*SmU | UU | OOSS |
| WV-45146 | mU*RfU*SmAmUmAfGmAmGmCmAmAmGmAfAmCfAmCmUmGn0 | UUAUAGAGCAAGAACACUGUU | RSOOOOOOOOOOOOOOOOn |
| | 01SmUmU*SmU*SmU | UU | SOSS |
| WV-46535 | mU*RfU*SmAmUmAfGmAmGmCmAmAmGmAfAmCfAmCmUmGmU | UUAUAGAGCAAGAACACUGUU | RSOOOOOOOOOOOOOOOOO |
| | n001SmU*SmU*SmU | UU | nSSS |
| WV-46536 | mU*RfU*SmAmUmAfGmAmGmCmAmAmGmAfAmCfAmCmUmGmU | UUAUAGAGCAAGAACACUGUU | RSOOOOOOOOOOOOOOOOO |
| | mUn001SmU*SmU | UU | OnSS |
| WV-46537 | mU*RfU*SmAmUmAfGmAmGmCmAmAmGmAfAmCfAmCmUmGmU | UUAUAGAGCAAGAACACUGUU | RSOOOOOOOOOOOOOOOOO |
| | mU*SmUn001SmU | UU | OSnS |
| WV-46538 | mUn001RfU*RmAmUmAfGmAmGmCmAmAmGmAfAmCfAmCmUm | UUAUAGAGCAAGAACACUGUU | nRROOOOOOOOOOOOOOOO |
| | GmUmU*SmU*SmU | UU | OOSS |
| WV-46539 | mU*SfUn001RmAmUmAfGmAmGmCmAmAmGmAfAmCfAmCmUm | UUAUAGAGCAAGAACACUGUU | SnROOOOOOOOOOOOOOOO |
| | GmUmU*SmU*SmU | UU | OOSS |
| WV-46540 | mU*SfU*RmAn001RmUmAfGmAmGmCmAmAmGmAfAmCfAmCm | UUAUAGAGCAAGAACACUGUU | SRnROOOOOOOOOOOOOOO |
| | UmGmUmU*SmU*SmU | UU | OOSS |
| WV-46541 | mU*SfU*RmAmUn001RmAfGmAmGmCmAmAmGmAfAmCfAmCm | UUAUAGAGCAAGAACACUGUU | SROnROOOOOOOOOOOOOO |
| | UmGmUmU*SmU*SmU | UU | OOSS |
| WV-46542 | mU*SfU*RmAmUmAn001RfGmAmGmCmAmAmGmAfAmCfAmCm | UUAUAGAGCAAGAACACUGUU | SROOnROOOOOOOOOOOOO |
| | UmGmUmU*SmU*SmU | UU | OOSS |
| WV-46543 | mU*SfU*RmAmUmAfGn001RmAmGmCmAmAmGmAfAmCfAmCm | UUAUAGAGCAAGAACACUGUU | SROOOnROOOOOOOOOOOO |
| | UmGmUmU*SmU*SmU | UU | OOSS |
| WV-46544 | mU*SfU*RmAmUmAfGmAn001RmGmCmAmAmGmAfAmCfAmCm | UUAUAGAGCAAGAACACUGUU | SROOOOnROOOOOOOOOOO |
| | UmGmUmU*SmU*SmU | UU | OOSS |
| WV-46545 | mU*SfU*RmAmUmAfGmAmGn001RmCmAmAmGmAfAmCfAmCm | UUAUAGAGCAAGAACACUGUU | SROOOOOnROOOOOOOOOO |
| | UmGmUmU*SmU*SmU | UU | OOSS |
| WV-46546 | mU*SfU*RmAmUmAfGmAmGmCn001RmAmAmGmAfAmCfAmCm | UUAUAGAGCAAGAACACUGUU | SROOOOOOnROOOOOOOOO |
| | UmGmUmU*SmU*SmU | UU | OOSS |
| WV-46547 | mU*SfU*RmAmUmAfGmAmGmCmAn001RmAmGmAfAmCfAmCm | UUAUAGAGCAAGAACACUGUU | SROOOOOOOnROOOOOOOO |
| | UmGmUmU*SmU*SmU | UU | OOSS |
| WV-46548 | mU*SfU*RmAmUmAfGmAmGmCmAmAn001RmGmAfAmCfAmCm | UUAUAGAGCAAGAACACUGUU | SROOOOOOOOnROOOOOOO |
| | UmGmUmU*SmU*SmU | UU | OOSS |
| WV-46549 | mU*SfU*RmAmUmAfGmAmGmCmAmAmGn001RmAfAmCfAmCm | UUAUAGAGCAAGAACACUGUU | SROOOOOOOOOnROOOOOO |
| | UmGmUmU*SmU*SmU | UU | OOSS |
| WV-46550 | mU*SfU*RmAmUmAfGmAmGmCmAmAmGmAn001RfAmCfAmCm | UUAUAGAGCAAGAACACUGUU | SROOOOOOOOOOnROOOOO |
| | UmGmUmU*SmU*SmU | UU | OOSS |
| WV-46551 | mU*SfU*RmAmUmAfGmAmGmCmAmAmGmAfAn001RmCfAmCm | UUAUAGAGCAAGAACACUGUU | SROOOOOOOOOOOnROOOO |
| | UmGmUmU*SmU*SmU | UU | OOSS |
| WV-46552 | mU*SfU*RmAmUmAfGmAmGmCmAmAmGmAfAmCn001RfAmCm | UUAUAGAGCAAGAACACUGUU | SROOOOOOOOOOOOnROOO |
| | UmGmUmU*SmU*SmU | UU | OOSS |
| WV-46553 | mU*SfU*RmAmUmAfGmAmGmCmAmAmGmAfAmCfAn001RmCm | UUAUAGAGCAAGAACACUGUU | SROOOOOOOOOOOOOnROO |
| | UmGmUmU*SmU*SmU | UU | OOSS |
| WV-46554 | mU*SfU*RmAmUmAfGmAmGmCmAmAmGmAfAmCfAmCn001Rm | UUAUAGAGCAAGAACACUGUU | SROOOOOOOOOOOOOOnRO |
| | UmGmUmU*SmU*SmU | UU | OOSS |
| WV-46555 | mU*SfU*RmAmUmAfGmAmGmCmAmAmGmAfAmCfAmCmUn001 | UUAUAGAGCAAGAACACUGUU | SROOOOOOOOOOOOOOOnR |
| | RmGmUmU*SmU*SmU | UU | OOSS |
| WV-46556 | mU*SfU*RmAmUmAfGmAmGmCmAmAmGmAfAmCfAmCmUmGn0 | UUAUAGAGCAAGAACACUGUU | SROOOOOOOOOOOOOOOOn |
| | 01RmUmU*SmU*SmU | UU | ROSS |
| WV-46557 | mU*SfU*RmAmUmAfGmAmGmCmAmAmGmAfAmCfAmCmUmGmU | UUAUAGAGCAAGAACACUGUU | SROOOOOOOOOOOOOOOOO |
| | n001RmU*SmU*SmU | UU | nRSS |
| WV-46558 | mU*SfU*RmAmUmAfGmAmGmCmAmAmGmAfAmCfAmCmUmGmU | UUAUAGAGCAAGAACACUGUU | SROOOOOOOOOOOOOOOOO |
| | mUn001RmU*SmU | UU | OnRS |
| WV-46559 | mU*SfU*RmAmUmAfGmAmGmCmAmAmGmAfAmCfAmCmUmGmU | UUAUAGAGCAAGAACACUGUU | SROOOOOOOOOOOOOOOOO |
| | mU*SmUn001RmU | UU | OSnR |
| WV-46560 | mUn001SfU*RmAmUmAfGmAmGmCmAmAmGmAfAmCfAmCmUm | UUAUAGAGCAAGAACACUGUU | nSROOOOOOOOOOOOOOOO |
| | GmUmU*SmU*SmU | UU | OOSS |
| WV-46561 | mU*SfUn001SmAmUmAfGmAmGmCmAmAmGmAfAmCfAmCmUm | UUAUAGAGCAAGAACACUGUU | SnSOOOOOOOOOOOOOOOO |
| | GmUmU*SmU*SmU | UU | OOSS |
| WV-44453 | mU*SfU*RmAn001SmUmAfGmAmGmCmAmAmGmAfAmCfAmCm | UUAUAGAGCAAGAACACUGUU | SRnSOOOOOOOOOOOOOOO |
| | UmGmUmU*SmU*SmU | UU | OOSS |
| WV-46562 | mU*SfU*RmAmUn001SmAfGmAmGmCmAmAmGmAfAmCfAmCm | UUAUAGAGCAAGAACACUGUU | SROnSOOOOOOOOOOOOOO |
| | UmGmUmU*SmU*SmU | UU | OOSS |
| WV-46563 | mU*SfU*RmAmUmAn001SfGmAmGmCmAmAmGmAfAmCfAmCm | UUAUAGAGCAAGAACACUGUU | SROOnSOOOOOOOOOOOOO |
| | UmGmUmU*SmU*SmU | UU | OOSS |
| WV-46564 | mU*SfU*RmAmUmAfGn001SmAmGmCmAmAmGmAfAmCfAmCm | UUAUAGAGCAAGAACACUGUU | SROOOnSOOOOOOOOOOOO |
| | UmGmUmU*SmU*SmU | UU | OOSS |
| WV-46565 | mU*SfU*RmAmUmAfGmAn001SmGmCmAmAmGmAfAmCfAmCm | UUAUAGAGCAAGAACACUGUU | SROOOOnSOOOOOOOOOOO |
| | UmGmUmU*SmU*SmU | UU | OOSS |
| WV-46566 | mU*SfU*RmAmUmAfGmAmGn001SmCmAmAmGmAfAmCfAmCm | UUAUAGAGCAAGAACACUGUU | SROOOOOnSOOOOOOOOOO |
| | UmGmUmU*SmU*SmU | UU | OOSS |
| WV-46567 | mU*SfU*RmAmUmAfGmAmGmCn001SmAmAmGmAfAmCfAmCm | UUAUAGAGCAAGAACACUGUU | SROOOOOOnSOOOOOOOOO |
| | UmGmUmU*SmU*SmU | UU | OOSS |
| WV-44452 | mU*SfU*RmAmUmAfGmAmGmCmAn001SmAmGmAfAmCfAmCm | UUAUAGAGCAAGAACACUGUU | SROOOOOOOnSOOOOOOOO |
| | UmGmUmU*SmU*SmU | UU | OOSS |
| WV-46568 | mU*SfU*RmAmUmAfGmAmGmCmAmAn001SmGmAfAmCfAmCm | UUAUAGAGCAAGAACACUGUU | SROOOOOOOOnSOOOOOOO |
| | UmGmUmU*SmU*SmU | UU | OOSS |
| WV-46569 | mU*SfU*RmAmUmAfGmAmGmCmAmAmGn001SmAfAmCfAmCm | UUAUAGAGCAAGAACACUGUU | SROOOOOOOOOn$OOOOOO |
| | UmGmUmU*SmU*SmU | UU | OOSS |
| WV-46570 | mU*SfU*RmAmUmAfGmAmGmCmAmAmGmAn001SfAmCfAmCm | UUAUAGAGCAAGAACACUGUU | SROOOOOOOOOOnSOOOOO |
| | UmGmUmU*SmU*SmU | UU | OOSS |
| WV-46571 | mU*SfU*RmAmUmAfGmAmGmCmAmAmGmAfAn001SmCfAmCm | UUAUAGAGCAAGAACACUGUU | SROOOOOOOOOOOnSOOOO |
| | UmGmUmU*SmU*SmU | UU | OOSS |
| WV-46572 | mU*SfU*RmAmUmAfGmAmGmCmAmAmGmAfAmCn001SfAmCm | UUAUAGAGCAAGAACACUGUU | SROOOOOOOOOOOOnSOOO |
| | UmGmUmU*SmU*SmU | UU | OOSS |
| WV-46573 | mU*SfU*RmAmUmAfGmAmGmCmAmAmGmAfAmCfAn001SmCm | UUAUAGAGCAAGAACACUGUU | SROOOOOOOOOOOOOnSOO |
| | UmGmUmU*SmU*SmU | UU | OOSS |

| | | | |
|---|---|---|---|
| WV-46574 | mU*SfU*RmAmUmAfGmAmGmCmAmAmGmAfAmCfAmCn001Sm | UUAUAGAGCAAGAACACUGUU | SROOOOOOOOOOOOOOnSO |
| | UmGmUmU*SmU*Sm | UU | OOSS |
| WV-46575 | mU*SfU*RmAmUmAfGmAmGmCmAmAmGmAfAmCfAmCmUn001 | UUAUAGAGCAAGAACACUGUU | SROOOOOOOOOOOOOOOnS |
| | SmGmUmU*SmU*SmU | UU | OOSS |
| WV-44451 | mU*SfU*RmAmUmAfGmAmGmCmAmAmGmAfAmCfAmCmUmGn0 | UUAUAGAGCAAGAACACUGUU | SROOOOOOOOOOOOOOOOn |
| | 01SmUmU*SmU*SmU | UU | SOSS |
| WV-46576 | mU*SfU*RmAmUmAfGmAmGmCmAmAmGmAfAmCfAmCmUmGmU | UUAUAGAGCAAGAACACUGUU | SROOOOOOOOOOOOOOOOO |
| | n001SmU*SmU*SmU | UU | nSSS |
| WV-46577 | mU*SfU*RmAmUmAfGmAmGmCmAmAmGmAfAmCfAmCmUmGmU | UUAUAGAGCAAGAACACUGUU | SROOOOOOOOOOOOOOOOO |
| | mUn001SmU*SmU | UU | OnSS |
| WV-44457 | mU*SfU*RmAmUmAfGmAmGmCmAmAmGmAfAmCfAmCmUmGmU | UUAUAGAGCAAGAACACUGUU | SROOOOOOOOOOOOOOOOO |
| | mU*SmUn001SmU | UU | OSnS |
| WV-43774 | mU*RfU*SmAmUmAfGmAmGmCmAmAmGmAfAmCfAmCmUmGmU | UUAUAGAGCAAGAACACUGUU | RSOOOOOOOOOOOOOOOOO |
| | mU*SmU*SmU | UU | OSS |
| WV-47066 | mU*RfUn001RfAmUmAfGmAmGmCmAmAmGmAfAmCfAmCmUm | UUAUAGAGCAAGAACACUGUU | RnROOOOOOOOOOOOOOOO |
| | GmUmU*SmU*SmU | UU | OOSS |
| WV-47067 | mU*RfU*SmAn001RfUmAfGmAmGmCmAmAmGmAfAmCfAmCm | UUAUAGAGCAAGAACACUGUU | RSnROOOOOOOOOOOOOOO |
| | UmGmUmU*SmU*SmU | UU | OOSS |
| WV-47068 | mU*RfU*SmAmUn001RfAfGmAmGmCmAmAmGmAfAmCfAmCm | UUAUAGAGCAAGAACACUGUU | RSOnROOOOOOOOOOOOOO |
| | UmGmUmU*SmU*SmU | UU | OOSS |
| WV-47069 | mU*RfU*SmAmUmAfGn001RfAmGmCmAmAmGmAfAmCfAmCm | UUAUAGAGCAAGAACACUGUU | RSOOOnROOOOOOOOOOOO |
| | UmGmUmU*SmU*SmU | UU | OOSS |
| WV-47070 | mU*RfU*SmAmUmAfGmAn001RfGmCmAmAmGmAfAmCfAmCm | UUAUAGAGCAAGAACACUGUU | RSOOOOnROOOOOOOOOOO |
| | UmGmUmU*SmU*SmU | UU | OOSS |
| WV-47071 | mU*RfU*SmAmUmAfGmAmGn001RfCmAmAmGmAfAmCfAmCm | UUAUAGAGCAAGAACACUGUU | RSOOOOOnROOOOOOOOOO |
| | UmGmUmU*SmU*SmU | UU | OOSS |
| WV-47072 | mU*RfU*SmAmUmAfGmAmGmCn001RfAmAmGmAfAmCfAmCm | UUAUAGAGCAAGAACACUGUU | RSOOOOOOnROOOOOOOOO |
| | UmGmUmU*SmU*SmU | UU | OOSS |
| WV-47073 | mU*RfU*SmAmUmAfGmAmGmCmAn001RfAmGmAfAmCfAmCm | UUAUAGAGCAAGAACACUGUU | RSOOOOOOOnROOOOOOOO |
| | UmGmUmU*SmU*SmU | UU | OOSS |
| WV-47074 | mU*RfU*SmAmUmAfGmAmGmCmAmAn001RfGmAfAmCfAmCm | UUAUAGAGCAAGAACACUGUU | RSOOOOOOOOnROOOOOOO |
| | UmGmUmU*SmU*SmU | UU | OOSS |
| WV-47075 | mU*RfU*SmAmUmAfGmAmGmCmAmAmGn001RfAfAmCfAmCm | UUAUAGAGCAAGAACACUGUU | RSOOOOOOOOOnROOOOOO |
| | UmGmUmU*SmU*SmU | UU | OOSS |
| WV-47076 | mU*RfU*SmAmUmAfGmAmGmCmAmAmGmAfAn001RfCfAmCm | UUAUAGAGCAAGAACACUGUU | RSOOOOOOOOOOOnROOOO |
| | UmGmUmU*SmU*SmU | UU | OOSS |
| WV-47077 | mU*RfU*SmAmUmAfGmAmGmCmAmAmGmAfAmCfAn001RfCm | UUAUAGAGCAAGAACACUGUU | RSOOOOOOOOOOOOOnROO |
| | UmGmUmU*SmU*SmU | UU | OOSS |
| WV-47078 | mU*RfU*SmAmUmAfGmAmGmCmAmAmGmAfAmCfAmCn001Rf | UUAUAGAGCAAGAACACUGUU | RSOOOOOOOOOOOOOOnRO |
| | UmGmUmU*SmU*SmU | UU | OOSS |
| WV-47079 | mU*RfU*SmAmUmAfGmAmGmCmAmAmGmAfAmCfAmCmUn001 | UUAUAGAGCAAGAACACUGUU | RSOOOOOOOOOOOOOOOnR |
| | RfGmUmU*SmU*SmU | UU | OOSS |
| WV-47080 | mU*RfU*SmAmUmAfGmAmGmCmAmAmGmAfAmCfAmCmUmGn0 | UUAUAGAGCAAGAACACUGUU | RSOOOOOOOOOOOOOOOOn |
| | 01RfUmU*SmU*SmU | UU | ROSS |
| WV-47081 | mU*RfU*SmAmUmAfGmAmGmCmAmAmGmAfAmCfAmCmUmGmU | UUAUAGAGCAAGAACACUGUU | RSOOOOOOOOOOOOOOOOO |
| | n001RfU*SmU*SmU | UU | nRSS |
| WV-47082 | mU*RfU*SmAmUmAfGmAmGmCmAmAmGmAfAmCfAmCmUmGmU | UUAUAGAGCAAGAACACUGUU | RSOOOOOOOOOOOOOOOOO |
| | mUn001RfU*SmU | UU | OnRS |
| WV-47083 | mU*RfU*SmAmUmAfGmAmGmCmAmAmGmAfAmCfAmCmUmGmU | UUAUAGAGCAAGAACACUGUU | RSOOOOOOOOOOOOOOOOO |
| | mU*SmUn001RfU | UU | OSnR |
| WV-47084 | mU*RfUn001SfAmUmAfGmAmGmCmAmAmGmAfAmCfAmCmUm | UUAUAGAGCAAGAACACUGUU | RnSOOOOOOOOOOOOOOOO |
| | GmUmU*SmU*SmU | UU | OOSS |
| WV-47085 | mU*RfU*SmAn001SfUmAfGmAmGmCmAmAmGmAfAmCfAmCm | UUAUAGAGCAAGAACACUGUU | RSnSOOOOOOOOOOOOOOO |
| | UmGmUmU*SmU*SmU | UU | OOSS |
| WV-47086 | mU*RfU*SmAmUn001SfAfGmAmGmCmAmAmGmAfAmCfAmCm | UUAUAGAGCAAGAACACUGUU | RSOnSOOOOOOOOOOOOOO |
| | UmGmUmU*SmU*SmU | UU | OOSS |
| WV-47087 | mU*RfU*SmAmUmAfGn001SfAmGmCmAmAmGmAfAmCfAmCm | UUAUAGAGCAAGAACACUGUU | RSOOOnSOOOOOOOOOOOO |
| | UmGmUmU*SmU*SmU | UU | OOSS |
| WV-47088 | mU*RfU*SmAmUmAfGmAn001SfGmCmAmAmGmAfAmCfAmCm | UUAUAGAGCAAGAACACUGUU | RSOOOOnSOOOOOOOOOOO |
| | UmGmUmU*SmU*SmU | UU | OOSS |
| WV-47089 | mU*RfU*SmAmUmAfGmAmGn001SfCmAmAmGmAfAmCfAmCm | UUAUAGAGCAAGAACACUGUU | RSOOOOOnSOOOOOOOOOO |
| | UmGmUmU*SmU*SmU | UU | OOSS |
| WV-47090 | mU*RfU*SmAmUmAfGmAmGmCn001SfAmAmGmAfAmCfAmCm | UUAUAGAGCAAGAACACUGUU | RSOOOOOOnSOOOOOOOOO |
| | UmGmUmU*SmU*SmU | UU | OOSS |
| WV-47091 | mU*RfU*SmAmUmAfGmAmGmCmAn001SfAmGmAfAmCfAmCm | UUAUAGAGCAAGAACACUGUU | RSOOOOOOOnSOOOOOOOO |
| | UmGmUmU*SmU*SmU | UU | OOSS |
| WV-47092 | mU*RfU*SmAmUmAfGmAmGmCmAmAn001SfGmAfAmCfAmCm | UUAUAGAGCAAGAACACUGUU | RSOOOOOOOOnSOOOOOOO |
| | UmGmUmU*SmU*SmU | UU | OOSS |
| WV-47093 | mU*RfU*SmAmUmAfGmAmGmCmAmAmGn001SfAfAmCfAmCm | UUAUAGAGCAAGAACACUGUU | RSOOOOOOOOOnSOOOOOO |
| | UmGmUmU*SmU*SmU | UU | OOSS |
| WV-47094 | mU*RfU*SmAmUmAfGmAmGmCmAmAmGmAfAn001SfCfAmCm | UUAUAGAGCAAGAACACUGUU | RSOOOOOOOOOOOn$OOOO |
| | UmGmUmU*SmU*SmU | UU | OOSS |
| WV-47095 | mU*RfU*SmAmUmAfGmAmGmCmAmAmGmAfAmCfAn001SfCm | UUAUAGAGCAAGAACACUGUU | RSOOOOOOOOOOOOOnSOO |
| | UmGmUmU*SmU*SmU | UU | OOSS |
| WV-47096 | mU*RfU*SmAmUmAfGmAmGmCmAmAmGmAfAmCfAmCn001Sf | UUAUAGAGCAAGAACACUGUU | RSOOOOOOOOOOOOOOnSO |
| | UmGmUmU*SmU*SmU | UU | OOSS |
| WV-47097 | mU*RfU*SmAmUmAfGmAmGmCmAmAmGmAfAmCfAmCmUn001 | UUAUAGAGCAAGAACACUGUU | RSOOOOOOOOOOOOOOOnS |
| | SfGmUmU*SmU*SmU | UU | OOSS |
| WV-47098 | mU*RfU*SmAmUmAfGmAmGmCmAmAmGmAfAmCfAmCmUmGn0 | UUAUAGAGCAAGAACACUGUU | RSOOOOOOOOOOOOOOOOn |
| | 01SfUmU*SmU*SmU | UU | SOSS |
| WV-47099 | mU*RfU*SmAmUmAfGmAmGmCmAmAmGmAfAmCfAmCmUmGmU | UUAUAGAGCAAGAACACUGUU | RSOOOOOOOOOOOOOOOOO |
| | n001SfU*SmU*SmU | UU | nSSS |
| WV-47100 | mU*RfU*SmAmUmAfGmAmGmCmAmAmGmAfAmCfAmCmUmGmU | UUAUAGAGCAAGAACACUGUU | RSOOOOOOOOOOOOOOOOO |
| | mUn001SfU*SmU | UU | OnSS |
| WV-47101 | mU*RfU*SmAmUmAfGmAmGmCmAmAmGmAfAmCfAmCmUmGmU | UUAUAGAGCAAGAACACUGUU | RSOOOOOOOOOOOOOOOOO |
| | mU*SmUn001SfU | UU | OSnS |
| WV-43775 | mU*SfU*RmAmUmAfGmAmGmCmAmAmGmAfAmCfAmCmUmGmU | UUAUAGAGCAAGAACACUGUU | SROOOOOOOOOOOOOOOOO |
| | mU*SmU*SmU | UU | OSS |
| WV-47102 | mU*SfUn001RfAm UmAfGmAmGmCmAmAmGmAfAmCfAmCmU | UUAUAGAGCAAGAACACUGUU | SnROOOOOOOOOOOOOOOO |
| | mGmUmU*SmU*SmU | UU | oOSS |
| WV-47103 | mU*SfU*RmAn001RfUmAfGmAmGmCmAmAmGmAfAmCfAmCm | UUAUAGAGCAAGAACACUGUU | SRnROOOOOOOOOOOOOOO |
| | UmGmUmU*SmU*SmU | UU | OOSS |
| WV-47104 | mU*SfU*RmAmUn001RfAfGmAmGmCmAmAmGmAfAmCfAmCm | UUAUAGAGCAAGAACACUGUU | SROnROOOOOOOOOOOOOO |
| | UmGmUmU*SmU*SmU | UU | OOSS |
| WV-47105 | mU*SfU*RmAmUmAfGn001RfAmGmCmAmAmGmAfAmCfAmCm | UUAUAGAGCAAGAACACUGUU | SROOOnROOOOOOOOOOOO |
| | UmGmUmU*SmU*SmU | UU | OOSS |
| WV-47106 | mU*SfU*RmAmUmAfGmAn001RfGmCmAmAmGmAfAmCfAmCm | UUAUAGAGCAAGAACACUGUU | SROOOOnROOOOOOOOOOO |
| | UmGmUmU*SmU*SmU | UU | OOSS |
| WV-47107 | mU*SfU*RmAmUmAfGmAmGn001RfCmAmAmGmAfAmCfAmCm | UUAUAGAGCAAGAACACUGUU | SROOOOOnROOOOOOOOOO |
| | UmGmUmU*SmU*SmU | UU | OOSS |
| WV-47108 | mU*SfU*RmAmUmAfGmAmGmCn001RfAmAmGmAfAmCfAmCm | UUAUAGAGCAAGAACACUGUU | SROOOOOOnROOOOOOOOO |
| | UmGmUmU*SmU*SmU | UU | OOSS |
| WV-47109 | mU*SfU*RmAmUmAfGmAmGmCmAn001RfAmGmAfAmCfAmCm | UUAUAGAGCAAGAACACUGUU | SROOOOOOOnROOOOOOOO |
| | UmGmUmU*SmU*SmU | UU | OOSS |
| WV-47110 | mU*SfU*RmAmUmAfGmAmGmCmAmAn001RfGmAfAmCfAmCm | UUAUAGAGCAAGAACACUGUU | SROOOOOOOOnROOOOOOO |
| | UmGmUmU*SmU*SmU | UU | OOSS |
| WV-47111 | mU*SfU*RmAmUmAfGmAmGmCmAmAmGn001RfAfAmCfAmCm | UUAUAGAGCAAGAACACUGUU | SROOOOOOOOOnROOOOOO |
| | UmGmUmU*SmU*SmU | UU | OOSS |
| WV-47112 | mU*SfU*RmAmUmAfGmAmGmCmAmAmGmAfAn001RfCfAmCm | UUAUAGAGCAAGAACACUGUU | SROOOOOOOOOOOnROOOO |
| | UmGmUmU*SmU*SmU | UU | OOSS |
| WV-47113 | mU*SfU*RmAmUmAfGmAmGmCmAmAmGmAfAmCfAn001RfCm | UUAUAGAGCAAGAACACUGUU | SROOOOOOOOOOOOOnROO |
| | UmGmUmU*SmU*SmU | UU | OOSS |
| WV-47114 | mU*SfU*RmAmUmAfGmAmGmCmAmAmGmAfAmCfAmCn001Rf | UUAUAGAGCAAGAACACUGUU | SROOOOOOOOOOOOOOnRO |
| | UmGmUmU*SmU*SmU | UU | OOSS |
| WV-47115 | mU*SfU*RmAmUmAfGmAmGmCmAmAmGmAfAmCfAmCmUn001 | UUAUAGAGCAAGAACACUGUU | SROOOOOOOOOOOOOOOnR |
| | RfGmUmU*SmU*SmU | UU | OOSS |
| WV-47116 | mU*SfU*RmAmUmAfGmAmGmCmAmAmGmAfAmCfAmCmUmGn0 | UUAUAGAGCAAGAACACUGUU | SROOOOOOOOOOOOOOOOn |
| | 01RfUmU*SmU*SmU | UU | ROSS |
| WV-47118 | mU*SfU*RmAmUmAfGmAmGmCmAmAmGmAfAmCfAmCmUmGmU | UUAUAGAGCAAGAACACUGUU | SROOOOOOOOOOOOOOOOO |
| | mUn001RfU*SmU | UU | OnRS |
| WV-47119 | mU*SfU*RmAmUmAfGmAmGmCmAmAmGmAfAmCfAmCmUmGmU | UUAUAGAGCAAGAACACUGUU | SROOOOOOOOOOOOOOOOO |
| | mU*SmUn001RfU | UU | OSnR |
| WV-47120 | mU*SfUn001SfAmUmAfGmAmGmCmAmAmGmAfAmCfAmCmUm | UUAUAGAGCAAGAACACUGUU | SnSOOOOOOOOOOOOOOOO |
| | GmUmU*SmU*SmU | UU | OOSS |
| WV-47121 | mU*SfU*RmAn001SfUmAfGmAmGmCmAmAmGmAfAmCfAmCm | UUAUAGAGCAAGAACACUGUU | SRnSOOOOOOOOOOOOOOO |
| | UmGmUmU*SmU*SmU | UU | OOSS |
| WV-47122 | mU*SfU*RmAmUn001SfAfGmAmGmCmAmAmGmAfAmCfAmCm | UUAUAGAGCAAGAACACUGUU | SROnSOOOOOOOOOOOOOO |
| | UmGmUmU*SmU*SmU | UU | OOSS |
| WV-47123 | mU*SfU*RmAmUmAfGn001SfAmGmCmAmAmGmAfAmCfAmCm | UUAUAGAGCAAGAACACUGUU | SROOOnSOOOOOOOOOOOO |
| | UmGmUmU*SmU*SmU | UU | OOSS |
| WV-47124 | mU*SfU*RmAmUmAfGmAn001SfGmCmAmAmGmAfAmCfAmCm | UUAUAGAGCAAGAACACUGUU | SROOOOnSOOOOOOOOOOO |
| | UmGmUmU*SmU*SmU | UU | OOSS |
| WV-47125 | mU*SfU*RmAmUmAfGmAmGn001SfCmAmAmGmAfAmCfAmCm | UUAUAGAGCAAGAACACUGUU | SROOOOOnSOOOOOOOOOO |
| | UmGmUmU*SmU*SmU | UU | OOSS |
| WV-47126 | mU*SfU*RmAmUmAfGmAmGmCn001SfAmAmGmAfAmCfAmCm | UUAUAGAGCAAGAACACUGUU | SROOOOOOnSOOOOOOOOO |
| | UmGmUmU*SmU*SmU | UU | OOSS |
| WV-47127 | mU*SfU*RmAmUmAfGmAmGmCmAn001SfAmGmAfAmCfAmCm | UUAUAGAGCAAGAACACUGUU | SROOOOOOOnSOOOOOOOO |
| | UmGmUmU*SmU*SmU | UU | OOSS |
| WV-47128 | mU*SfU*RmAmUmAfGmAmGmCmAmAn001SfGmAfAmCfAmCm | UUAUAGAGCAAGAACACUGUU | SROOOOOOOOnSOOOOOOO |
| | UmGmUmU*SmU*SmU | UU | OOSS |
| WV-47129 | mU*SfU*RmAmUmAfGmAmGmCmAmAmGn001SfAfAmCfAmCm | UUAUAGAGCAAGAACACUGUU | SROOOOOOOOOnSOOOOOO |
| | UmGmUmU*SmU*SmU | UU | OOSS |
| WV-47130 | mU*SfU*RmAmUmAfGmAmGmCmAmAmGmAfAn001SfCfAmCm | UUAUAGAGCAAGAACACUGUU | SROOOOOOOOOOOnSOOOO |
| | UmGmUmU*SmU*SmU | UU | OOSS |
| WV-47131 | mU*SfU*RmAmUmAfGmAmGmCmAmAmGmAfAmCfAn001SfCm | UUAUAGAGCAAGAACACUGUU | SROOOOOOOOOOOOOnSOO |
| | UmGmUmU*SmU*SmU | UU | OOSS |
| WV-47132 | mU*SfU*RmAmUmAfGmAmGmCmAmAmGmAfAmCfAmCn001Sf | UUAUAGAGCAAGAACACUGUU | SROOOOOOOOOOOOOOnSO |
| | UmGmUmU*SmU*SmU | UU | OOSS |
| WV-47133 | mU*SfU*RmAmUmAfGmAmGmCmAmAmGmAfAmCfAmCmUn001 | UUAUAGAGCAAGAACACUGUU | SROOOOOOOOOOOOOOOnS |
| | SfGmUmU*SmU*SmU | UU | OOSS |
| WV-47134 | mU*SfU*RmAmUmAfGmAmGmCmAmAmGmAfAmCfAmCmUmGn0 | UUAUAGAGCAAGAACACUGUU | SROOOOOOOOOOOOOOOOn |
| | 01SfUmU*SmU*SmU | UU | SOSS |
| WV-47135 | mU*SfU*RmAmUmAfGmAmGmCmAmAmGmAfAmCfAmCmUmGmU | UUAUAGAGCAAGAACACUGUU | SROOOOOOOOOOOOOOOOO |
| | n001SfU*SmU*SmU | UU | nSSS |
| WV-47136 | mU*SfU*RmAmUmAfGmAmGmCmAmAmGmAfAmCfAmCmUmGmU | UUAUAGAGCAAGAACACUGUU | SROOOOOOOOOOOOOOOOO |
| | mUn001SfU*SmU | UU | OnSS |
| WV-47137 | mU*SfU*RmAmUmAfGmAmGmCmAmAmGmAfAmCfAmCmUmGmU | UUAUAGAGCAAGAACACUGUU | SROOOOOOOOOOOOOOOOO |
| | mU*SmUn001SfU | UU | OSnS |
| WV-41826 | mU*fU*mAmUmAfGmAmGmCmAmAmGmAfAmCfAmCmUmGmUmU | UUAUAGAGCAAGAACACUGUU | XXOOOOOOOOOOOOOOOOO |
| | *mU*mU | UU | OXX |
| WV-41828 | Mod001L001mAm*AmCmAmGmUfGmUfUfCfUmUmGmCmUmCm | AACAGUGUUCUUGCUCUAUAA | OXOOOOOOOOOOOOOOOOO |
| | UmAmUmA*mA | | OX |
| WV-46380 | T*SfU*RmAmUmAfGmAmGmCmAmAmGmAfAmCfAmCmUmGmUm | TUAUAGAGCAAGAACACUGUU | SROOOOOOOOOOOOOOOOO |
| | U*SmU*SmU | UU | OSS |
| WV-46381 | POT*SfU*RmAmUmAfGmAmGmCmAmAmGmAfAmCfAmCmUmGm | TUAUAGAGCAAGAACACUGUU | SROOOOOOOOOOOOOOOOO |
| | UmU*SmU*SmU | UU | OSS |
| WV-46382 | PO5MSdT*SfU*RmAmUmAfGmAmGmCmAmAmGmAfAmCfAmCm | TUAUAGAGCAAGAACACUGUU | SROOOOOOOOOOOOOOOOO |
| | UmGmUmU*SmU*SmU | UU | OSS |
| WV-46383 | PO5MRdT*SfU*RmAmUmAfGmAmGmCmAmAmGmAfAmCfAmCm | TUAUAGAGCAAGAACACUGUU | SROOOOOOOOOOOOOOOOO |
| | UmGmUmU*SmU*SmU | UU | OSS |
| WV-46384 | VPT*SfU*RmAmUmAfGmAmGmCmAmAmGmAfAmCfAmCmUmGm | TUAUAGAGCAAGAACACUGUU | SROOOOOOOOOOOOOOOOO |
| | UmU*SmU*SmU | UU | OSS |
| WV-46385 | 5mspdT*SfU*RmAmUmAfGmAmGmCmAmAmGmAfAmCfAmCmU | TUAUAGAGCAAGAACACUGUU | SROOOOOOOOOOOOOOOOO |
| | mGmUmU*SmU*SmU | UU | OSS |
| WV-46386 | 5mrpdT*SfU*RmAmUmAfGmAmGmCmAmAmGmAfAmCfAmCmU | TUAUAGAGCAAGAACACUGUU | SROOOOOOOOOOOOOOOOO |
| | mGmUmU*SmU*SmU | UU | OSS |
| WV-42079 | mU*SfU*RmAn001SmUmAfGmAmGmCmAn001SmAmGmAfAmC | UUAUAGAGCAAGAACACUGUU | SRnSOOOOOOnSOOOOOOO |
| | fAmCmUmGn001SmUmU*SmU*SmU | UU | OnSOSS |
| WV-44434 | T*SfU*RmAn001SmUmAfGmAmGmCmAn001SmAmGmAfAmCf | TUAUAGAGCAAGAACACUGUU | SRnSOOOOOOnSOOOOOOO |
| | AmCmUmGn001SmUmU*SmU*SmU | UU | OnSOSS |
| WV-44435 | POT*SfU*RmAn001SmUmAfGmAmGmCmAn001SmAmGmAfAm | TUAUAGAGCAAGAACACUGUU | SRnSOOOOOOnSOOOOOOO |
| | CfAmCmUmGn001SmUmU*SmU*SmU | UU | OnSOSS |
| WV-44436 | PO5MSdT*SfU*RmAn001SmUmAfGmAmGmCmAn001SmAmGm | TUAUAGAGCAAGAACACUGUU | SRnSOOOOOOnSOOOOOOO |
| | AfAmCfAmCmUmGn001SmUmU*SmU*SmU | UU | OnSOSS |
| WV-44437 | PO5MRdT*SfU*RmAn001SmUmAfGmAmGmCmAn001SmAmGm | TUAUAGAGCAAGAACACUGUU | SRnSOOOOOOn$OOOOOOO |
| | AfAmCfAmCmUmGn001SmUmU*SmU*SmU | UU | OnSOSS |
| WV-44438 | VPT*SfU*RmAn001SmUmAfGmAmGmCmAn001SmAmGmAfAm | TUAUAGAGCAAGAACACUGUU | SRnSOOOOOOnSOOOOOOO |
| | CfAmCmUmGn001SmUmU*SmU*SmU | UU | OnSOSS |
| WV-44439 | 5mvpdT*SfU*RmAn001SmUmAfGmAmGmCmAn001SmAmGmA | TUAUAGAGCAAGAACACUGUU | SRnSOOOOOOnSOOOOOOO |
| | fAmCfAmCmUmGn001SmUmU*SmU*SmU | UU | OnSOSS |
| WV-44440 | 5mspdT*SfU*RmAn001SmUmAfGmAmGmCmAn001SmAmGmA | TUAUAGAGCAAGAACACUGUU | SRnSOOOOOOnSOOOOOOO |
| | fAmCfAmCmUmGn001SmUmU*SmU*SmU | UU | OnSOSS |
| WV-44441 | 5mrpdT*SfU*RmAn001SmUmAfGmAmGmCmAn001SmAmGmA | TUAUAGAGCAAGAACACUGUU | SRnSOOOOOOnSOOOOOOO |
| | fAmCfAmCmUmGn001SmUmU*SmU*SmU | UU | OnSOSS |
| WV-46387 | T*RfU*SmAmUmAfGmAmGmCmAmAmGmAfAmCfAmCmUmGmUm | TUAUAGAGCAAGAACACUGUU | RSOOOOOOOOOOOOOOOOO |
| | U*SmU*SmU | UU | OSS |
| WV-46388 | POT*RfU*SmAmUmAfGmAmGmCmAmAmGmAfAmCfAmCmUmGm | TUAUAGAGCAAGAACACUGUU | RSOOOOOOOOOOOOOOOOO |
| | UmU*SmU*SmU | UU | OSS |
| WV-46389 | PO5MSdT*RfU*SmAmUmAfGmAmGmCmAmAmGmAfAmCfAmCm | TUAUAGAGCAAGAACACUGUU | RSOOOOOOOOOOOOOOOOO |
| | UmGmUmU*SmU*SmU | UU | OSS |
| WV-46390 | PO5MRdT*RfU*SmAmUmAfGmAmGmCmAmAmGmAfAmCfAmCm | TUAUAGAGCAAGAACACUGUU | RSOOOOOOOOOOOOOOOOO |
| | UmGmUmU*SmU*SmU | UU | OSS |
| WV-46391 | VPT*RfU*SmAmUmAfGmAmGmCmAmAmGmAfAmCfAmCmUmGm | TUAUAGAGCAAGAACACUGUU | RSOOOOOOOOOOOOOOOOO |
| | UmU*SmU*SmU | UU | OSS |
| WV-46392 | 5mspdT*RfU*SmAmUmAfGmAmGmCmAmAmGmAfAmCfAmCmU | TUAUAGAGCAAGAACACUGUU | RSOOOOOOOOOOOOOOOOO |
| | mGmUmU*SmU*SmU | UU | OSS |
| WV-46393 | 5mrpdT*RfU*SmAmUmAfGmAmGmCmAmAmGmAfAmCfAmCmU | TUAUAGAGCAAGAACACUGUU | RSOOOOOOOOOOOOOOOOO |
| | mGmUmU*SmU*SmU | UU | OSS |
| WV-42078 | mU*RfU*SmAn001SmUmAfGmAmGmCmAn001SmAmGmAfAmC | UUAUAGAGCAAGAACACUGUU | RSnSOOOOOOnSOOOOOOO |
| | fAmCmUmGn001SmUmU*SmU*SmU | UU | OnSOSS |
| WV-46394 | T*RfU*SmAn001SmUmAfGmAmGmCmAn001SmAmGmAfAmCf | TUAUAGAGCAAGAACACUGUU | RSnSOOOOOOnSOOOOOOO |
| | AmCmUmGn001SmUmU*SmU*SmU | UU | OnSOSS |
| WV-46395 | POT*RfU*SmAn001SmUmAfGmAmGmCmAn001SmAmGmAfAm | TUAUAGAGCAAGAACACUGUU | RSnSOOOOOOnSOOOOOOO |
| | CfAmCmUmGn001SmUmU*SmU*SmU | UU | OnSOSS |
| WV-46396 | PO5MSdT*RfU*SmAn001SmUmAfGmAmGmCmAn001SmAmGm | TUAUAGAGCAAGAACACUGUU | RSSOOOOOOnSOOOOOOOO |
| | AfAmCfAmCmUmGn001SmUmU*SmU*SmU | UU | nSOSS |
| WV-46397 | PO5MRdT*RfU*SmAn001SmUmAfGmAmGmCmAn001SmAmGm | TUAUAGAGCAAGAACACUGUU | RSSOOOOOOnSOOOOOOOO |
| | AfAmCfAmCmUmGn001SmUmU*SmU*SmU | UU | nSOSS |
| WV-46398 | VPT*RfU*SmAn001SmUmAfGmAmGmCmAn001SmAmGmAfAm | TUAUAGAGCAAGAACACUGUU | RSnSOOOOOOnSOOOOOOO |
| | CfAmCmUmGn001SmUmU*SmU*SmU | UU | OnSOSS |
| WV-46399 | 5mspdT*RfU*SmAn001SmUmAfGmAmGmCmAn001SmAmGmA | TUAUAGAGCAAGAACACUGUU | RSnSOOOOOOnSOOOOOOO |
| | fAmCfAmCmUmGn001SmUmU*SmU*SmU | UU | OnSOSS |
| WV-46400 | 5mrpdT*RfU*SmAn001SmUmAfGmAmGmCmAn001SmAmGmA | TUAUAGAGCAAGAACACUGUU | RSnSOOOOOOnSOOOOOOO |
| | fAmCfAmCmUmGn001SmUmU*SmU*SmU | UU | OnSOSS |
| WV-42080 | Mod001L001mA*SmAmCmAmGmUfGmUfUfCfUmUmGmCmUmC | AACAGUGUUCUUGCUCUAUAA | OSOOOOOOOOOOOOOOOOO |
| | mUmAmUmA*SmA | | OS |
| WV-47144 | mU*RfU*SmAn001SfUmAfGmAmGmCmAn001SfAmGmAfAmC | UUAUAGAGCAAGAACACUGUU | RSnSOOOOOOnSOOOOOOO |
| | fAmCmUmGmUmU*SmU*SmU | UU | OOOSS |
| WV-47145 | mU*SfU*RmAn001SfUmAfGmAmGmCmAn001SfAmGmAfAmC | UUAUAGAGCAAGAACACUGUU | SRnSOOOOOOnSOOOOOOO |
| | fAmCmUmGmUmU*SmU*SmU | UU | OOOSS |
| WV-43771 | mU*SfU*RmAmUmAfGmAfGfCmAmAmGmAfAmCfAmCmUmGmU | UUAUAGAGCAAGAACACUGUU | SROOOOOOOOOOOOOOOOO |
| | mU*SmU*SmU | UU | OSS |
| WV-43773 | mU*SfU*RmAn001SmUmAfGmAfGfCmAn001SmAmGmAfAmC | UUAUAGAGCAAGAACACUGUU | SRnSOOOOOOnSOOOOOOO |
| | fAmCmUmGn001SmUmU*SmU*SmU | UU | OnSOSS |
| WV-43988 | mU*SfU*RmAn001SfUmAfGmAmGmCmAn001SfAmGmAfAmC | UUAUAGAGCAAGAACACUGUU | SRnSOOOOOOnSOOOOOOO |
| | fAmCmUmGn001SfUmU*SmU*SmU | UU | OnSOSS |
| WV-43989 | mU*SfU*RmAn001SfUmAfGmAmGmCfAn001SmAmGmAfAmC | UUAUAGAGCAAGAACACUGUU | SRnSOOOOOOnSOOOOOOO |
| | fAmCmUmGn001SfUmU*SmU*SmU | UU | OnSOSS |
| WV-43994 | mU*SfU*RfAn001SfUmAfGmAfGmCfAn001SfAmGmAfAmC | UUAUAGAGCAAGAACACUGUU | SRnSOOOOOOnSOOOOOOO |
| | fAmCfUfGn001SfUmU*SmU*SmU | UU | OnSOSS |
| WV-43996 | mU*SfU*RmAn001SfUmAfGmAfGfCmAn001SfAmGmAfAmC | UUAUAGAGCAAGAACACUGUU | SRnSOOOOOOnSOOOOOOO |
| | fAmCmUmGn001SfUmU*SmU*SmU | UU | OnSOSS |
| WV-43256 | mU*SfU*RmAn001SfUmAfGmAfGmCfAn001SmAmGmAfAmC | UUAUAGAGCAAGAACACUGUU | SRnSOOOOOOnSOOOOOOO |
| | fAmCfUmGn001SfUmU*SmU*SmU | UU | OnSOSS |
| WV-46991 | mU*fU*mAfUmAfGmAmGmCmAfAmGmAfAmCfAmCmUmGfUmU | UUAUAGAGCAAGAACACUGUU | XXOOOOOOOOOOOOOOOOO |
| | *mU*mU | UU | OXX |
| WV-46992 | mU*SfU*RmAfUmAfGmAmGmCmAfAmGmAfAmCfAmCmUmGfU | UUAUAGAGCAAGAACACUGUU | SROOOOOOOOOOOOOOOOO |
| | mU*SmU*SmU | UU | OSS |
| WV-46993 | T*SfU*RmAn001SfUmAfGmAmGmCmAn001SfAmGmAfAmCf | TUAUAGAGCAAGAACACUGUU | SRnSOOOOOOnSOOOOOOO |
| | AmCmUmGn001SfUmU*SmU*SmU | UU | OnSOSS |
| WV-46994 | POT*SfU*RmAn001SfUmAfGmAmGmCmAn001SfAmGmAfAm | TUAUAGAGCAAGAACACUGUU | SRnSOOOOOOnSOOOOOOO |
| | CfAmCmUmGn001SfUmU*SmU*SmU | UU | OnSOSS |
| WV-46995 | PO5MSdT*SfU*RmAn001SfUmAfGmAmGmCmAn001SfAmGm | TUAUAGAGCAAGAACACUGUU | SRnSOOOOOOnSOOOOOOO |
| | AfAmCfAmCmUmGn001SfUmU*SmU*SmU | UU | OnSOSS |
| WV-46996 | PO5MRdT*SfU*RmAn001SfUmAfGmAmGmCmAn001SfAmGm | TUAUAGAGCAAGAACACUGUU | SRnSOOOOOOnSOOOOOOO |
| | AfAmCfAmCmUmGn001SfUmU*SmU*SmU | UU | OnSOSS |
| WV-46997 | VPT*SfU*RmAn001SfUmAfGmAmGmCmAn001SfAmGmAfAm | TUAUAGAGCAAGAACACUGUU | SRnSOOOOOOnSOOOOOOO |
| | CfAmCmUmGn001SfUmU*SmU*SmU | UU | OnSOSS |
| WV-46998 | 5mspdT*SfU*RmAn001SfUmAfGmAmGmCmAn001SfAmGmA | TUAUAGAGCAAGAACACUGUU | SRnSOOOOOOnSOOOOOOO |
| | fAmCfAmCmUmGn001SfUmU*SmU*SmU | UU | OnsOSS |
| WV-46999 | 5mrpdT*SfU*RmAn001SfUmAfGmAmGmCmAn001SfAmGmA | TUAUAGAGCAAGAACACUGUU | SRnSOOOOOOnSOOOOOOO |
| | fAmCfAmCmUmGn001SfUmU*SmU*SmU | UU | OnSOSS |
| WV-47000 | mU*RfU*SmAfUmAfGmAmGmCmAfAmGmAfAmCfAmCmUmGfU | UUAUAGAGCAAGAACACUGUU | RSOOOOOOOOOOOOOOOOO |
| | mU*SmU*SmU | UU | OSS |
| WV-47001 | mU*RfU*SmAn001SfUmAfGmAmGmCmAn001SfAmGmAfAmC | UUAUAGAGCAAGAACACUGUU | RSnSOOOOOOnSOOOOOOO |
| | fAmCmUmGn001SfUmU*SmU*SmU | UU | OnSOSS |
| WV-47002 | T*RfU*SmAn001SfUmAfGmAmGmCmAn001SfAmGmAfAmCf | TUAUAGAGCAAGAACACUGUU | RSnSOOOOOOnSOOOOOOO |
| | AmCmUmGn001SfUmU*SmU*SmU | UU | OnSOSS |
| WV-47003 | POT*RfU*SmAn001SfUmAfGmAmGmCmAn001SfAmGmAfAm | TUAUAGAGCAAGAACACUGUU | RSnSOOOOOOnSOOOOOOO |
| | CfAmCmUmGn001SfUmU*SmU*SmU | UU | OnSOSS |
| WV-47004 | PO5MSdT*RfU*SmAn001SfUmAfGmAmGmCmAn001SfAmGm | TUAUAGAGCAAGAACACUGUU | RSnSOOOOOOnSOOOOOOO |
| | AfAmCfAmCmUmGn001SfUmU*SmU*SmU | UU | OnSOSS |
| WV-47005 | PO5MRdT*RfU*SmAn001SfUmAfGmAmGmCmAn001SfAmGm | TUAUAGAGCAAGAACACUGUU | RSnSOOOOOOnSOOOOOOO |
| | AfAmCfAmCmUmGn001SfUmU*SmU*SmU | UU | OnSOSS |
| WV-47006 | VPT*RfU*SmAn001SfUmAfGmAmGmCmAn001SfAmGmAfAm | TUAUAGAGCAAGAACACUGUU | RSnSOOOOOOnSOOOOOOO |
| | CfAmCmUmGn001SfUmU*SmU*SmU | UU | OnSOSS |
| WV-47007 | 5mspdT*RfU*SmAn001SfUmAfGmAmGmCmAn001SfAmGmA | TUAUAGAGCAAGAACACUGUU | RSnSOOOOOOnSOOOOOOO |
| | fAmCfAmCmUmGn001SfUmU*SmU*SmU | UU | OnSOSS |
| WV-47008 | 5mrpdT*RfU*SmAn001SfUmAfGmAmGmCmAn001SfAmGmA | TUAUAGAGCAAGAACACUGUU | RSnSOOOOOOnSOOOOOOO |
| | fAmCfAmCmUmGn001SfUmU*SmU*SmU | UU | OnSOSS |
| WV-47009 | mU*fU*mAfUmAfGmAfGfCmAfAmGmAfAmCfAmCmUmGfUmU | UUAUAGAGCAAGAACACUGUU | XXOOOOOOOOOOOOOOOOO |
| | *mU*mU | UU | OXX |
| WV-47010 | mU*SfU*RmAfUmAfGmAfGfCmAfAmGmAfAmCfAmCmUmGfU | UUAUAGAGCAAGAACACUGUU | SROOOOOOOOOOOOOOOOO |
| | mU*SmU*SmU | UU | OSS |
| WV-47011 | T*SfU*RmAn001SfUmAfGmAfGfCmAn001SfAmGmAfAmCf | TUAUAGAGCAAGAACACUGUU | SRnSOOOOOOnSOOOOOOO |
| | AmCmUmGn001SfUmU*SmU*SmU | UU | OnSOSS |
| WV-47012 | POT*SfU*RmAn001SfUmAfGmAfGfCmAn001SfAmGmAfAm | TUAUAGAGCAAGAACACUGUU | SRnSOOOOOOnSOOOOOOO |
| | CfAmCmUmGn001SfUmU*SmU*SmU | UU | OnSOSS |
| WV-47013 | PO5MSdT*SfU*RmAn001SfUmAfGmAfGfCmAn001SfAmGm | TUAUAGAGCAAGAACACUGUU | SRnSOOOOOOnSOOOOOOO |
| | AfAmCfAmCmUmGn001SfUmU*SmU*SmU | UU | OnSOSS |
| WV-47014 | PO5MRdT*SfU*RmAn001SfUmAfGmAfGfCmAn001SfAmGm | TUAUAGAGCAAGAACACUGUU | SRnSOOOOOOnSOOOOOOO |
| | AfAmCfAmCmUmGn001SfUmU*SmU*SmU | UU | OnSOSS |
| WV-47015 | VPT*SfU*RmAn001SfUmAfGmAfGfCmAn001SfAmGmAfAm | TUAUAGAGCAAGAACACUGUU | SRnSOOOOOOnSOOOOOOO |
| | CfAmCmUmGn001SfUmU*SmU*SmU | UU | OnSOSS |
| WV-47016 | 5mspdT*SfU*RmAn001SfUmAfGmAfGfCmAn001SfAmGmA | TUAUAGAGCAAGAACACUGUU | SRnSOOOOOOnSOOOOOOO |
| | fAmCfAmCmUmGn001SfUmU*SmU*SmU | UU | OnSOSS |
| WV-47017 | 5mrpdT*SfU*RmAn001SfUmAfGmAfGfCmAn001SfAmGmA | TUAUAGAGCAAGAACACUGUU | SRnSOOOOOOnSOOOOOOO |
| | fAmCfAmCmUmGn001SfUmU*SmU*SmU | UU | OnSOSS |
| WV-47018 | mU*RfU*SmAfUmAfGmAfGfCmAfAmGmAfAmCfAmCmUmGfU | UUAUAGAGCAAGAACACUGUU | RSOOOOOOOOOOOOOOOOO |
| | mU*SmU*SmU | UU | OSS |
| WV-47019 | mU*RfU*SmAn001SfUmAfGmAfGfCmAn001SfAmGmAfAmC | UUAUAGAGCAAGAACACUGUU | RSnSOOOOOOnSOOOOOOO |
| | fAmCmUmGn001SfUmU*SmU*SmU | UU | OnSOSS |
| WV-47020 | T*RfU*SmAn001SfUmAfGmAfGfCmAn001SfAmGmAfAmCf | TUAUAGAGCAAGAACACUGUU | RSnSOOOOOOnSOOOOOOO |
| | AmCmUmGn001SfUmU*SmU*SmU | UU | OnSOSS |
| WV-47021 | POT*RfU*SmAn001SfUmAfGmAfGfCmAn001SfAmGmAfAm | TUAUAGAGCAAGAACACUGUU | RSnSOOOOOOnSOOOOOOO |
| | CfAmCmUmGn001SfUmU*SmU*SmU | UU | OnSOSS |
| WV-47022 | PO5MSdT*RfU*SmAn001SfUmAfGmAfGfCmAn001SfAmGm | TUAUAGAGCAAGAACACUGUU | RSnSOOOOOOnSOOOOOOO |
| | AfAmCfAmCmUmGn001SfUmU*SmU*SmU | UU | OnSOSS |
| WV-47023 | PO5MRdT*RfU*SmAn001SfUmAfGmAfGfCmAn001SfAmGm | TUAUAGAGCAAGAACACUGUU | RSnSOOOOOOnSOOOOOOO |
| | AfAmCfAmCmUmGn001SfUmU*SmU*SmU | UU | OnSOSS |
| WV-47024 | VPT*RfU*SmAn001SfUmAfGmAfGfCmAn001SfAmGmAfAm | TUAUAGAGCAAGAACACUGUU | RSnSOOOOOOnSOOOOOOO |
| | CfAmCmUmGn001SfUmU*SmU*SmU | UU | OnSOSS |
| WV-47025 | 5mspdT*RfU*SmAn001SfUmAfGmAfGfCmAn001SfAmGmA | TUAUAGAGCAAGAACACUGUU | RSnSOOOOOOnSOOOOOOO |
| | fAmCfAmCmUmGn001SfUmU*SmU*SmU | UU | OnSOSS |
| WV-47026 | 5mrpdT*RfU*SmAn001SfUmAfGmAfGfCmAn001SfAmGmA | TUAUAGAGCAAGAACACUGUU | RSnSOOOOOOnSOOOOOOO |
| | fAmCfAmCmUmGn001SfUmU*SmU*SmU | UU | OnsOSS |
| WV-48526 | mU*RfU*SmAn001SfUmAfGmAfGmCfAn001RfAmGmAfAmC | UUAUAGAGCAAGAACACUGUU | RSnSOOOOOOnROOOOOOO |
| | fAmCfUmGn001RfUmU*SmU*SmU | UU | OnROSS |
| WV-48527 | mU*SfU*RmAn001SfUmAfGmAfGmCfAn001RfAmGmAfAmC | UUAUAGAGCAAGAACACUGUU | SRnSOOOOOOnROOOOOOO |
| | fAmCfUmGn001RfUmU*SmU*SmU | UU | OnROSS |
| WV-48528 | mU*SfU*RmAn001SfUmAfGmAmGmCmAn001RfAmGmAfAmC | UUAUAGAGCAAGAACACUGUU | SRnSOOOOOOnROOOOOOO |
| | fAmCmUmGmUmU*SmU*SmU | UU | OOOSS |
| WV-48529 | mU*SfU*RmAn001SfUmAfGmAmGmCmAn001RfAmGmAfAmC | UUAUAGAGCAAGAACACUGUU | SRnSOOOOOOnROOOOOOO |
| | fAmCmUmGn001RfUmU*SmU*SmU | UU | OnROSS |
| WV-48530 | mU*RfU*SmAmUmAfGmAmGmCmAn001RfAmGmAfAmCfAmCm | UUAUAGAGCAAGAACACUGUU | RSOOOOOOOnROOOOOOOO |
| | UmGn001RfUmU*SmU*SmU | UU | nROSS |
| WV-48531 | mU*SfU*RmAmUmAfGmAmGmCmAn001RfAmGmAfAmCfAmCm | UUAUAGAGCAAGAACACUGUU | SROOOOOOOnROOOOOOOO |
| | UmGn001RfUmU*SmU*SmU | UU | nROSS |
| WV-20167 | mU*fU*mAfUmAfGmAfGmCfAmAmGmAfAmCfAmCfUmGfUmU | UUAUAGAGCAAGAACACUGUU | XXOOOOOOOOOOOOOOOOO |
| | *mU*mU | UU | OXX |
| WV-20170 | mU*RfU*SmAfUmAfGmAfGmCfAmAmGmAfAmCfAmCfUmGfU | UUAUAGAGCAAGAACACUGUU | RSOOOOOOOOOOOOOOOOO |
| | mU*SmU*SmU | UU | OSS |
| WV-38708 | mU*RfU*SmAn001SfUmAfGmAfGmCfAn001SmAmGmAfAmC | UUAUAGAGCAAGAACACUGUU | RSnSOOOOOOnSOOOOOOO |
| | fAmCfUmGn001SfUmU*SmU*SmU | UU | OnSOSS |
| WV-36860 | Mod001L001mAmAfCmAfGmUfGmUfUfCfUmUfGmCfUmCfU | AACAGUGUUCUUGCUCUAUAA | OOOOOOOOOOOOOOOOOOO |
| | mAfUmAfA*mU*mU | UU | OOXX |
| WV-36807 | Mod001L001mAmAfCmAfGmUfGmUfUfCfUmUfGmCfUmCfU | AACAGUGUUCUUGCUCUAUAA | OOOOOOOOOOOOOOOOOOO |
| | mAfUmAfA*SmU*SmU | UU | OOSS |
| WV-20167 | mU*fU*mAfUmAfGmAfGmCfAmAmGmAfAmCfAmCfUmGfUmU | UUAUAGAGCAAGAACACUGUU | XXOOOOOOOOOOOOOOOOO |
| | *mU*mU | UU | OXX |
| WV-20169 | mU*SfU*SmAfUmAfGmAfGmCfAmAmGmAfAmCfAmCfUmGfU | UUAUAGAGCAAGAACACUGUU | SSOOOOOOOOOOOOOOOOO |
| | mU*SmU*SmU | UU | OSS |
| WV-20170 | mU*RfU*SmAfUmAfGmAfGmCfAmAmGmAfAmCfAmCfUmGfU | UUAUAGAGCAAGAACACUGUU | RSOOOOOOOOOOOOOOOOO |
| | mU*SmU*SmU | UU | OSS |
| WV-20171 | mU*SfU*RmAfUmAfGmAfGmCfAmAmGmAfAmCfAmCfUmGfU | UUAUAGAGCAAGAACACUGUU | SROOOOOOOOOOOOOOOOO |
| | mU*SmU*SmU | UU | OSS |
| WV-20172 | mU*RfU*RmAfUmAfGmAfGmCfAmAmGmAfAmCfAmCfUmGfU | UUAUAGAGCAAGAACACUGUU | RROOOOOOOOOOOOOOOOO |
| | mU*SmU*SmU | UU | OSS |
| WV-20183 | mU*SfU*RmAfUmAfGmAfGmCfAmAmGmAfAmCfAmCfUmGfU | UUAUAGAGCAAGAACACUGUU | SROOOOOOOOOOOOOOOOO |
| | mU*RmU*RmU | UU | ORR |
| WV-36836 | mUn001fU*mAfUmAfGmAfGmCfAmAmGmAfAmCfAmCfUmGf | UUAUAGAGCAAGAACACUGUU | nXXOOOOOOOOOOOOOOOO |
| | UmU*mU*mU | UU | OOXX |
| WV-36837 | mU*fUn001mAfUmAfGmAfGmCfAmAmGmAfAmCfAmCfUmGf | UUAUAGAGCAAGAACACUGUU | XnXOOOOOOOOOOOOOOOO |
| | UmU*mU*mU | UU | OOXX |
| WV-36838 | mU*fU*mAn001fUmAfGmAfGmCfAmAmGmAfAmCfAmCfUmG | UUAUAGAGCAAGAACACUGUU | XXXOOOOOOOOOOOOOOOO |
| | fUmU*mU*mU | UU | OXX |
| WV-36839 | mU*fU*mAfUn001mAfGmAfGmCfAmAmGmAfAmCfAmCfUmG | UUAUAGAGCAAGAACACUGUU | XXOnXOOOOOOOOOOOOOO |
| | fUmU*mU*mU | UU | OOXX |
| WV-36840 | mU*fU*mAfUmAn001fGmAfGmCfAmAmGmAfAmCfAmCfUmG | UUAUAGAGCAAGAACACUGUU | XXOOnXOOOOOOOOOOOOO |
| | fUmU*mU*mU | UU | OOXX |
| WV-36841 | mU*fU*mAfUmAfGn001mAfGmCfAmAmGmAfAmCfAmCfUmG | UUAUAGAGCAAGAACACUGUU | XXOOOnXOOOOOOOOOOOO |
| | fUmU*mU*mU | UU | OOXX |
| WV-36842 | mU*fU*mAfUmAfGmAn001fGmCfAmAmGmAfAmCfAmCfUmG | UUAUAGAGCAAGAACACUGUU | XXOOOOnXOOOOOOOOOOO |
| | fUmU*mU*mU | UU | OOXX |
| WV-36843 | mU*fU*mAfUmAfGmAfGn001mCfAmAmGmAfAmCfAmCfUmG | UUAUAGAGCAAGAACACUGUU | XXOOOOOnXOOOOOOOOOO |
| | fUmU*mU*mU | UU | OOXX |
| WV-36844 | mU*fU*mAfUmAfGmAfGmCn001fAmAmGmAfAmCfAmCfUmG | UUAUAGAGCAAGAACACUGUU | XXOOOOOOnXOOOOOOOOO |
| | fUmU*mU*mU | UU | OOXX |
| WV-36845 | mU*fU*mAfUmAfGmAfGmCfAn001mAmGmAfAmCfAmCfUmG | UUAUAGAGCAAGAACACUGUU | XXOOOOOOOnXOOOOOOOO |
| | fUmU*mU*mU | UU | OOXX |
| WV-36846 | mU*fU*mAfUmAfGmAfGmCfAmAn001mGmAfAmCfAmCfUmG | UUAUAGAGCAAGAACACUGUU | XXOOOOOOOOnxOOOOOOO |
| | fUmU*mU*mU | UU | OOXX |
| WV-36847 | mU*fU*mAfUmAfGmAfGmCfAmAmGn001mAfAmCfAmCfUmG | UUAUAGAGCAAGAACACUGUU | XXOOOOOOOOOnXOOOOOO |
| | fUmU*mU*mU | UU | OOXX |
| WV-36848 | mU*fU*mAfUmAfGmAfGmCfAmAmGmAn001fAmCfAmCfUmG | UUAUAGAGCAAGAACACUGUU | XXOOOOOOOOOOnXOOOOO |
| | fUmU*mU*mU | UU | OOXX |
| WV-36849 | mU*fU*mAfUmAfGmAfGmCfAmAmGmAfAn001mCfAmCfUmG | UUAUAGAGCAAGAACACUGUU | XXOOOOOOOOOOOnXOOOO |
| | fUmU*mU*mU | UU | OOXX |
| WV-36850 | mU*fU*mAfUmAfGmAfGmCfAmAmGmAfAmCn001fAmCfUmG | UUAUAGAGCAAGAACACUGUU | XXOOOOOOOOOOOOnxOOO |
| | fUmU*mU*mU | UU | OOXX |
| WV-36851 | mU*fU*mAfUmAfGmAfGmCfAmAmGmAfAmCfAn001mCfUmG | UUAUAGAGCAAGAACACUGUU | XXOOOOOOOOOOOOOnXOO |
| | fUmU*mU*mU | UU | OOXX |
| WV-36852 | mU*fU*mAfUmAfGmAfGmCfAmAmGmAfAmCfAmCn001fUmG | UUAUAGAGCAAGAACACUGUU | XXOOOOOOOOOOOOOOnXO |
| | fUmU*mU*mU | UU | OOXX |
| WV-36853 | mU*fU*mAfUmAfGmAfGmCfAmAmGmAfAmCfAmCfUn001mG | UUAUAGAGCAAGAACACUGUU | XXOOOOOOOOOOOOOOOnX |
| | fUmU*mU*mU | UU | OOXX |
| WV-36854 | mU*fU*mAfUmAfGmAfGmCfAmAmGmAfAmCfAmCfUmGn001 | UUAUAGAGCAAGAACACUGUU | XXOOOOOOOOOOOOOOOOn |
| | fUmU*mU*mU | UU | XOXX |
| WV-36855 | mU*fU*mAfUmAfGmAfGmCfAmAmGmAfAmCfAmCfUmGfUn0 | UUAUAGAGCAAGAACACUGUU | XXOOOOOOOOOOOOOOOOO |
| | 01mU*mU*mU | UU | nXXX |
| WV-36856 | mU*fU*mAfUmAfGmAfGmCfAmAmGmAfAmCfAmCfUmGfUmU | UUAUAGAGCAAGAACACUGUU | XXOOOOOOOOOOOOOOOOO |
| | n001mU*mU | UU | OnXX |
| WV-36857 | mU*fU*mAfUmAfGmAfGmCfAmAmGmAfAmCfAmCfUmGfUmU | UUAUAGAGCAAGAACACUGUU | XXOOOOOOOOOOOOOOOOO |
| | *mUn001mU | UU | OOXnX |
| WV-36980 | mU*RfU*RmA*RfU*RmA*RfG*RmA*RfG*SmCfAmAmGmAfA | UUAUAGAGCAAGAACACUGUU | RRRRRRRSOOOOOOOOOOO |
| | mCfAmCfUmGfUmU*SmU*SmU | UU | OSS |
| WV-36981 | mU*RfU*RmA*RfU*RmA*RfG*RmA*SfG*RmCfAmAmGmAfA | UUAUAGAGCAAGAACACUGUU | RRRRRRSROOOOOOOOOOO |
| | mCfAmCfUmGfUmU*SmU*SmU | UU | OSS |
| WV-36982 | mU*RfU*RmA*RfU*RmA*RfG*RmA*SfG*SmCfAmAmGmAfA | UUAUAGAGCAAGAACACUGUU | RRRRRRSSOOOOOOOOOOO |
| | mCfAmCfUmGfUmU*SmU*SmU | UU | OSS |
| WV-36983 | mU*RfU*RmA*RfU*RmA*RfG*SmA*RfG*RmCfAmAmGmAfA | UUAUAGAGCAAGAACACUGUU | RRRRRSRROOOOOOOOOOO |
| | mCfAmCfUmGfUmU*SmU*SmU | UU | OSS |
| WV-36984 | mU*RfU*RmA*RfU*RmA*RfG*SmA*RfG*SmCfAmAmGmAfA | UUAUAGAGCAAGAACACUGUU | RRRRRSRSOOOOOOOOOOO |
| | mCfAmCfUmGfUmU*SmU*SmU | UU | OSS |
| WV-36985 | mU*RfU*RmA*RfU*RmA*RfG*SmA*SfG*RmCfAmAmGmAfA | UUAUAGAGCAAGAACACUGUU | RRRRRSSROOOOOOOOOOO |
| | mCfAmCfUmGfUmU*SmU*SmU | UU | OSS |
| WV-36986 | mU*RfU*RmA*RfU*RmA*RfG*SmA*SfG*SmCfAmAmGmAfA | UUAUAGAGCAAGAACACUGUU | RRRRRSSSOOOOOOOOOOO |
| | mCfAmCfUmGfUmU*SmU*SmU | UU | OSS |
| WV-36987 | mU*RfU*RmA*RfU*RmA*SfG*RmA*RfG*RmCfAmAmGmAfA | UUAUAGAGCAAGAACACUGUU | RRRRSRRROOOOOOOOOOO |
| | mCfAmCfUmGfUmU*SmU*SmU | UU | OSS |
| WV-36988 | U*RfU*RmA*RfU*RmA*SfG*RmA*RfG*SmCfAmAmGmAfAm | UUAUAGAGCAAGAACACUGUU | RRRRSRRSOOOOOOOOOOO |
| | CfAmCfUmGfUmU*SmU*SmU | UU | OSS |
| WV-36989 | mU*RfU*RmA*RfU*RmA*SfG*RmA*SfG*RmCfAmAmGmAfA | UUAUAGAGCAAGAACACUGUU | RRRRSRSROOOOOOOOOOO |
| | mCfAmCfUmGfUmU*SmU*SmU | UU | OSS |
| WV-36990 | mU*RfU*RmA*RfU*RmA*SfG*RmA*SfG*SmCfAmAmGmAfA | UUAUAGAGCAAGAACACUGUU | RRRRSRSSOOOOOOOOOOO |
| | mCfAmCfUmGfUmU*SmU*SmU | UU | OSS |
| WV-36991 | mU*RfU*RmA*RfU*RmA*SfG*SmA*RfG*RmCfAmAmGmAfA | UUAUAGAGCAAGAACACUGUU | RRRRSSRROOOOOOOOOOO |
| | mCfAmCfUmGfUmU*SmU*SmU | UU | OSS |
| WV-36992 | mU*RfU*RmA*RfU*RmA*SfG*SmA*RfG*SmCfAmAmGmAfA | UUAUAGAGCAAGAACACUGUU | RRRRSSRSOOOOOOOOOOO |
| | mCfAmCfUmGfUmU*SmU*SmU | UU | OSS |
| WV-36993 | mU*RfU*RmA*RfU*RmA*SfG*SmA*SfG*RmCfAmAmGmAfA | UUAUAGAGCAAGAACACUGUU | RRRRSSSROOOOOOOOOOO |
| | mCfAmCfUmGfUmU*SmU*SmU | UU | OSS |
| WV-36994 | mU*RfU*RmA*RfU*RmA*SfG*SmA*SfG*SmCfAmAmGmAfA | UUAUAGAGCAAGAACACUGUU | RRRRSSSSOOOOOOOOOOO |
| | mCfAmCfUmGfUmU*SmU*SmU | UU | OSS |
| WV-36995 | mU*RfU*RmA*RfU*SmA*RfG*RmA*RfG*RmCfAmAmGmAfA | UUAUAGAGCAAGAACACUGUU | RRRSRRRROOOOOOOOOOO |
| | mCfAmCfUmGfUmU*SmU*SmU | UU | OSS |
| WV-36996 | mU*RfU*RmA*RfU*SmA*RfG*RmA*RfG*SmCfAmAmGmAfA | UUAUAGAGCAAGAACACUGUU | RRRSRRRSOOOOOOOOOOO |
| | mCfAmCfUmGfUmU*SmU*SmU | UU | OSS |
| WV-36997 | mU*RfU*RmA*RfU*SmA*RfG*RmA*SfG*RmCfAmAmGmAfA | UUAUAGAGCAAGAACACUGUU | RRRSRRSROOOOOOOOOOO |
| | mCfAmCfUmGfUmU*SmU*SmU | UU | OSS |
| WV-36998 | mU*RfU*RmA*RfU*SmA*RfG*RmA*SfG*SmCfAmAmGmAfA | UUAUAGAGCAAGAACACUGUU | RRRSRRSSOOOOOOOOOOO |
| | mCfAmCfUmGfUmU*SmU*SmU | UU | OSS |
| WV-36999 | mU*RfU*RmA*RfU*SmA*RfG*SmA*RfG*RmCfAmAmGmAfA | UUAUAGAGCAAGAACACUGUU | RRRSRSRROOOOOOOOOOO |
| | mCfAmCfUmGfUmU*SmU*SmU | UU | OSS |
| WV-37000 | mU*RfU*RmA*RfU*SmA*RfG*SmA*RfG*SmCfAmAmGmAfA | UUAUAGAGCAAGAACACUGUU | RRRSRSRSOOOOOOOOOOO |
| | mCfAmCfUmGfUmU*SmU*SmU | UU | OSS |
| WV-37001 | mU*RfU*RmA*RfU*SmA*RfG*SmA*SfG*RmCfAmAmGmAfA | UUAUAGAGCAAGAACACUGUU | RRRSRSSROOOOOOOOOOO |
| | mCfAmCfUmGfUmU*SmU*SmU | UU | OSS |
| WV-37002 | mU*RfU*RmA*RfU*SmA*RfG*SmA*SfG*SmCfAmAmGmAfA | UUAUAGAGCAAGAACACUGUU | RRRSRSSSOOOOOOOOOOO |
| | mCfAmCfUmGfUmU*SmU*SmU | UU | OSS |
| WV-37003 | mU*RfU*RmA*RfU*SmA*SfG*RmA*RfG*RmCfAmAmGmAfA | UUAUAGAGCAAGAACACUGUU | RRRSSRRROOOOOOOOOOO |
| | mCfAmCfUmGfUmU*SmU*SmU | UU | OSS |
| WV-37004 | mU*RfU*RmA*RfU*SmA*SfG*RmA*RfG*SmCfAmAmGmAfA | UUAUAGAGCAAGAACACUGUU | RRRSSRRSOOOOOOOOOOO |
| | mCfAmCfUmGfUmU*SmU*SmU | UU | OSS |
| WV-37005 | mU*RfU*RmA*RfU*SmA*SfG*RmA*SfG*RmCfAmAmGmAfA | UUAUAGAGCAAGAACACUGUU | RRRSSRSROOOOOOOOOOO |
| | mCfAmCfUmGfUmU*SmU*SmU | UU | OSS |
| WV-37006 | mU*RfU*RmA*RfU*SmA*SfG*RmA*SfG*SmCfAmAmGmAfA | UUAUAGAGCAAGAACACUGUU | RRRSSRSSOOOOOOOOOOO |
| | mCfAmCfUmGfUmU*SmU*SmU | UU | OSS |
| WV-37007 | mU*RfU*RmA*RfU*SmA*SfG*SmA*RfG*RmCfAmAmGmAfA | UUAUAGAGCAAGAACACUGUU | RRRSSSRROOOOOOOOOOO |
| | mCfAmCfUmGfUmU*SmU*SmU | UU | OSS |
| WV-37008 | mU*RfU*RmA*RfU*SmA*SfG*SmA*RfG*SmCfAmAmGmAfA | UUAUAGAGCAAGAACACUGUU | RRRSSSRSOOOOOOOOOOO |
| | mCfAmCfUmGfUmU*SmU*SmU | UU | OSS |
| WV-37009 | mU*RfU*RmA*RfU*SmA*SfG*SmA*SfG*RmCfAmAmGmAfA | UUAUAGAGCAAGAACACUGUU | RRRSSSSROOOOOOOOOOO |
| | mCfAmCfUmGfUmU*SmU*SmU | UU | OSS |
| WV-37010 | mU*RfU*RmA*RfU*SmA*SfG*SmA*SfG*SmCfAmAmGmAfA | UUAUAGAGCAAGAACACUGUU | RRRSSSSSOOOOOOOOOOO |
| | mCfAmCfUmGfUmU*SmU*SmU | UU | OSS |
| WV-37011 | mU*RfU*RmA*SfU*RmA*RfG*RmA*RfG*RmCfAmAmGmAfA | UUAUAGAGCAAGAACACUGUU | RRSRRRRROOOOOOOOOOO |
| | mCfAmCfUmGfUmU*SmU*SmU | UU | OSS |
| WV-37012 | mU*RfU*RmA*SfU*RmA*RfG*RmA*RfG*SmCfAmAmGmAfA | UUAUAGAGCAAGAACACUGUU | RRSRRRRSOOOOOOOOOOO |
| | mCfAmCfUmGfUmU*SmU*SmU | UU | OSS |
| WV-37013 | mU*RfU*RmA*SfU*RmA*RfG*RmA*SfG*RmCfAmAmGmAfA | UUAUAGAGCAAGAACACUGUU | RRSRRRSROOOOOOOOOOO |
| | mCfAmCfUmGfUmU*SmU*SmU | UU | OSS |
| WV-37014 | mU*RfU*RmA*SfU*RmA*RfG*RmA*SfG*SmCfAmAmGmAfA | UUAUAGAGCAAGAACACUGUU | RRSRRRSSOOOOOOOOOOO |
| | mCfAmCfUmGfUmU*SmU*SmU | UU | OSS |
| WV-37015 | mU*RfU*RmA*SfU*RmA*RfG*SmA*RfG*RmCfAmAmGmAfA | UUAUAGAGCAAGAACACUGUU | RRSRRSRROOOOOOOOOOO |
| | mCfAmCfUmGfUmU*SmU*SmU | UU | OSS |
| WV-37016 | mU*RfU*RmA*SfU*RmA*RfG*SmA*RfG*SmCfAmAmGmAfA | UUAUAGAGCAAGAACACUGUU | RRSRRSRSOOOOOOOOOOO |
| | mCfAmCfUmGfUmU*SmU*SmU | UU | OSS |
| WV-37017 | mU*RfU*RmA*SfU*RmA*RfG*SmA*SfG*RmCfAmAmGmAfA | UUAUAGAGCAAGAACACUGUU | RRSRRSSROOOOOOOOOOO |
| | mCfAmCfUmGfUmU*SmU*SmU | UU | OSS |
| WV-37018 | mU*RfU*RmA*SfU*RmA*RfG*SmA*SfG*SmCfAmAmGmAfA | UUAUAGAGCAAGAACACUGUU | RRSRRSSSOOOOOOOOOOO |
| | mCfAmCfUmGfUmU*SmU*SmU | UU | OSS |
| WV-37019 | mU*RfU*RmA*SfU*RmA*SfG*RmA*RfG*RmCfAmAmGmAfA | UUAUAGAGCAAGAACACUGUU | RRSRSRRROOOOOOOOOOO |
| | mCfAmCfUmGfUmU*SmU*SmU | UU | OSS |
| WV-37020 | mU*RfU*RmA*SfU*RmA*SfG*RmA*RfG*SmCfAmAmGmAfA | UUAUAGAGCAAGAACACUGUU | RRSRSRRSOOOOOOOOOOO |
| | mCfAmCfUmGfUmU*SmU*SmU | UU | OSS |
| WV-37021 | mU*RfU*RmA*SfU*RmA*SfG*RmA*SfG*RmCfAmAmGmAfA | UUAUAGAGCAAGAACACUGUU | RRSRSRSROOOOOOOOOOO |
| | mCfAmCfUmGfUmU*SmU*SmU | UU | OSS |
| WV-37022 | mU*RfU*RmA*SfU*RmA*SfG*RmA*SfG*SmCfAmAmGmAfA | UUAUAGAGCAAGAACACUGUU | RRSRSRSSOOOOOOOOOOO |
| | mCfAmCfUmGfUmU*SmU*SmU | UU | OSS |
| WV-37023 | mU*RfU*RmA*SfU*RmA*SfG*SmA*RfG*RmCfAmAmGmAfA | UUAUAGAGCAAGAACACUGUU | RRSRSSRROOOOOOOOOOO |
| | mCfAmCfUmGfUmU*SmU*SmU | UU | OSS |
| WV-37024 | mU*RfU*RmA*SfU*RmA*SfG*SmA*RfG*SmCfAmAmGmAfA | UUAUAGAGCAAGAACACUGUU | RRSRSSRSOOOOOOOOOOO |
| | mCfAmCfUmGfUmU*SmU*SmU | UU | OSS |
| WV-37025 | mU*RfU*RmA*SfU*RmA*SfG*SmA*SfG*RmCfAmAmGmAfA | UUAUAGAGCAAGAACACUGUU | RRSRSSSROOOOOOOOOOO |
| | mCfAmCfUmGfUmU*SmU*SmU | UU | OSS |
| WV-37026 | mU*RfU*RmA*SfU*RmA*SfG*SmA*SfG*SmCfAmAmGmAfA | UUAUAGAGCAAGAACACUGUU | RRSRSSSSOOOOOOOOOOO |
| | mCfAmCfUmGfUmU*SmU*SmU | UU | OSS |
| WV-37027 | mU*RfU*RmA*SfU*SmA*RfG*RmA*RfG*RmCfAmAmGmAfA | UUAUAGAGCAAGAACACUGUU | RRSSRRRROOOOOOOOOOO |
| | mCfAmCfUmGfUmU*SmU*SmU | UU | OSS |
| WV-37028 | mU*RfU*RmA*SfU*SmA*RfG*RmA*RfG*SmCfAmAmGmAfA | UUAUAGAGCAAGAACACUGUU | RRSSRRRSOOOOOOOOOOO |
| | mCfAmCfUmGfUmU*SmU*SmU | UU | OSS |
| WV-37029 | mU*RfU*RmA*SfU*SmA*RfG*RmA*SfG*RmCfAmAmGmAfA | UUAUAGAGCAAGAACACUGUU | RRSSRRSROOOOOOOOOOO |
| | mCfAmCfUmGfUmU*SmU*SmU | UU | OSS |
| WV-37030 | mU*RfU*RmA*SfU*SmA*RfG*RmA*SfG*SmCfAmAmGmAfA | UUAUAGAGCAAGAACACUGUU | RRSSRRSSOOOOOOOOOOO |
| | mCfAmCfUmGfUmU*SmU*SmU | UU | OSS |
| WV-37031 | mU*RfU*RmA*SfU*SmA*RfG*SmA*RfG*RmCfAmAmGmAfA | UUAUAGAGCAAGAACACUGUU | RRSSRSRROOOOOOOOOOO |
| | mCfAmCfUmGfUmU*SmU*SmU | UU | OSS |
| WV-37032 | mU*RfU*RmA*SfU*SmA*RfG*SmA*RfG*SmCfAmAmGmAfA | UUAUAGAGCAAGAACACUGUU | RRSSRSRSOOOOOOOOOOO |
| | mCfAmCfUmGfUmU*SmU*SmU | UU | OSS |
| WV-37033 | mU*RfU*RmA*SfU*SmA*RfG*SmA*SfG*RmCfAmAmGmAfA | UUAUAGAGCAAGAACACUGUU | RRSSRSSROOOOOOOOOOO |
| | mCfAmCfUmGfUmU*SmU*SmU | UU | OSS |
| WV-37034 | mU*RfU*RmA*SfU*SmA*RfG*SmA*SfG*SmCfAmAmGmAfA | UUAUAGAGCAAGAACACUGUU | RRSSRSSSOOOOOOOOOOO |
| | mCfAmCfUmGfUmU*SmU*SmU | UU | OSS |
| WV-37035 | mU*RfU*RmA*SfU*SmA*SfG*RmA*RfG*RmCfAmAmGmAfA | UUAUAGAGCAAGAACACUGUU | RRSSSRRROOOOOOOOOOO |
| | mCfAmCfUmGfUmU*SmU*SmU | UU | OSS |
| WV-37036 | mU*RfU*RmA*SfU*SmA*SfG*RmA*RfG*SmCfAmAmGmAfA | UUAUAGAGCAAGAACACUGUU | RRSSSRRSOOOOOOOOOOO |
| | mCfAmCfUmGfUmU*SmU*SmU | UU | OSS |
| WV-37037 | mU*RfU*RmA*SfU*SmA*SfG*RmA*SfG*RmCfAmAmGmAfA | UUAUAGAGCAAGAACACUGUU | RRSSSRSROOOOOOOOOOO |
| | mCfAmCfUmGfUmU*SmU*SmU | UU | OSS |
| WV-37038 | mU*RfU*RmA*SfU*SmA*SfG*RmA*SfG*SmCfAmAmGmAfA | UUAUAGAGCAAGAACACUGUU | RRSSSRSSOOOOOOOOOOO |
| | mCfAmCfUmGfUmU*SmU*SmU | UU | OSS |
| WV-37039 | mU*RfU*RmA*SfU*SmA*SfG*SmA*RfG*RmCfAmAmGmAfA | UUAUAGAGCAAGAACACUGUU | RRSSSSRROOOOOOOOOOO |
| | mCfAmCfUmGfUmU*SmU*SmU | UU | OSS |
| WV-37040 | mU*RfU*RmA*SfU*SmA*SfG*SmA*RfG*SmCfAmAmGmAfA | UUAUAGAGCAAGAACACUGUU | RRSSSSRSOOOOOOOOOOO |
| | mCfAmCfUmGfUmU*SmU*SmU | UU | OSS |
| WV-37041 | mU*RfU*RmA*SfU*SmA*SfG*SmA*SfG*RmCfAmAmGmAfA | UUAUAGAGCAAGAACACUGUU | RRSSSSSROOOOOOOOOOO |
| | mCfAmCfUmGfUmU*SmU*SmU | UU | OSS |
| WV-37042 | mU*RfU*RmA*SfU*SmA*SfG*SmA*SfG*SmCfAmAmGmAfA | UUAUAGAGCAAGAACACUGUU | RRSSSSSSOOOOOOOOOOO |
| | mCfAmCfUmGfUmU*SmU*SmU | UU | OSS |
| WV-37043 | mU*RfU*SmA*RfU*RmA*RfG*RmA*RfG*RmCfAmAmGmAfA | UUAUAGAGCAAGAACACUGUU | RSRRRRRROOOOOOOOOOO |
| | mCfAmCfUmGfUmU*SmU*SmU | UU | OSS |
| WV-37044 | mU*RfU*SmA*RfU*RmA*RfG*RmA*RfG*SmCfAmAmGmAfA | UUAUAGAGCAAGAACACUGUU | RSRRRRRSOOOOOOOOOOO |
| | mCfAmCfUmGfUmU*SmU*SmU | UU | OSS |
| WV-37045 | mU*RfU*SmA*RfU*RmA*RfG*RmA*SfG*RmCfAmAmGmAfA | UUAUAGAGCAAGAACACUGUU | RSRRRRSROOOOOOOOOOO |
| | mCfAmCfUmGfUmU*SmU*SmU | UU | OSS |
| WV-37046 | mU*RfU*SmA*RfU*RmA*RfG*RmA*SfG*SmCfAmAmGmAfA | UUAUAGAGCAAGAACACUGUU | RSRRRRSSOOOOOOOOOOO |
| | mCfAmCfUmGfUmU*SmU*SmU | UU | OSS |
| WV-37047 | mU*RfU*SmA*RfU*RmA*RfG*SmA*RfG*RmCfAmAmGmAfA | UUAUAGAGCAAGAACACUGUU | RSRRRSRROOOOOOOOOOO |
| | mCfAmCfUmGfUmU*SmU*SmU | UU | OSS |
| WV-37048 | mU*RfU*SmA*RfU*RmA*RfG*SmA*RfG*SmCfAmAmGmAfA | UUAUAGAGCAAGAACACUGUU | RSRRRSRSOOOOOOOOOOO |
| | mCfAmCfUmGfUmU*SmU*SmU | UU | OSS |
| WV-37049 | mU*RfU*SmA*RfU*RmA*RfG*SmA*SfG*RmCfAmAmGmAfA | UUAUAGAGCAAGAACACUGUU | RSRRRSSROOOOOOOOOOO |
| | mCfAmCfUmGfUmU*SmU*SmU | UU | OSS |
| WV-37050 | mU*RfU*SmA*RfU*RmA*RfG*SmA*SfG*SmCfAmAmGmAfA | UUAUAGAGCAAGAACACUGUU | RSRRRSSSOOOOOOOOOOO |
| | mCfAmCfUmGfUmU*SmU*SmU | UU | OSS |
| WV-37051 | mU*RfU*SmA*RfU*RmA*SfG*RmA*RfG*RmCfAmAmGmAfA | UUAUAGAGCAAGAACACUGUU | RSRRSRRROOOOOOOOOOO |
| | mCfAmCfUmGfUmU*SmU*SmU | UU | OSS |
| WV-37052 | mU*RfU*SmA*RfU*RmA*SfG*RmA*RfG*SmCfAmAmGmAfA | UUAUAGAGCAAGAACACUGUU | RSRRSRRSOOOOOOOOOOO |
| | mCfAmCfUmGfUmU*SmU*SmU | UU | OSS |
| WV-37053 | mU*RfU*SmA*RfU*RmA*SfG*RmA*SfG*RmCfAmAmGmAfA | UUAUAGAGCAAGAACACUGUU | RSRRSRSROOOOOOOOOOO |
| | mCfAmCfUmGfUmU*SmU*SmU | UU | OSS |
| WV-37054 | mU*RfU*SmA*RfU*RmA*SfG*RmA*SfG*SmCfAmAmGmAfA | UUAUAGAGCAAGAACACUGUU | RSRRSRSSOOOOOOOOOOO |
| | mCfAmCfUmGfUmU*SmU*SmU | UU | OSS |
| WV-37055 | mU*RfU*SmA*RfU*RmA*SfG*SmA*RfG*RmCfAmAmGmAfA | UUAUAGAGCAAGAACACUGUU | RSRRSSRROOOOOOOOOOO |
| | mCfAmCfUmGfUmU*SmU*SmU | UU | OSS |
| WV-37056 | mU*RfU*SmA*RfU*RmA*SfG*SmA*RfG*SmCfAmAmGmAfA | UUAUAGAGCAAGAACACUGUU | RSRRSSRSOOOOOOOOOOO |
| | mCfAmCfUmGfUmU*SmU*SmU | UU | OSS |
| WV-37057 | mU*RfU*SmA*RfU*RmA*SfG*SmA*SfG*RmCfAmAmGmAfA | UUAUAGAGCAAGAACACUGUU | RSRRSSSROOOOOOOOOOO |
| | mCfAmCfUmGfUmU*SmU*SmU | UU | OSS |
| WV-37058 | mU*RfU*SmA*RfU*RmA*SfG*SmA*SfG*SmCfAmAmGmAfA | UUAUAGAGCAAGAACACUGUU | RSRRSSSSOOOOOOOOOOO |
| | mCfAmCfUmGfUmU*SmU*SmU | UU | OSS |
| WV-37059 | mU*RfU*SmA*RfU*SmA*RfG*RmA*RfG*RmCfAmAmGmAfA | UUAUAGAGCAAGAACACUGUU | RSRSRRRROOOOOOOOOOO |
| | mCfAmCfUmGfUmU*SmU*SmU | UU | OSS |
| WV-37060 | mU*RfU*SmA*RfU*SmA*RfG*RmA*RfG*SmCfAmAmGmAfA | UUAUAGAGCAAGAACACUGUU | RSRSRRRSOOOOOOOOOOO |
| | mCfAmCfUmGfUmU*SmU*SmU | UU | OSS |
| WV-37061 | mU*RfU*SmA*RfU*SmA*RfG*RmA*SfG*RmCfAmAmGmAfA | UUAUAGAGCAAGAACACUGUU | RSRSRRSROOOOOOOOOOO |
| | mCfAmCfUmGfUmU*SmU*SmU | UU | OSS |
| WV-37062 | mU*RfU*SmA*RfU*SmA*RfG*RmA*SfG*SmCfAmAmGmAfA | UUAUAGAGCAAGAACACUGUU | RSRSRRSSOOOOOOOOOOO |
| | mCfAmCfUmGfUmU*SmU*SmU | UU | OSS |
| WV-37063 | mU*RfU*SmA*RfU*SmA*RfG*SmA*RfG*RmCfAmAmGmAfA | UUAUAGAGCAAGAACACUGUU | RSRSRSRROOOOOOOOOOO |
| | mCfAmCfUmGfUmU*SmU*SmU | UU | OSS |
| WV-37064 | mU*RfU*SmA*RfU*SmA*RfG*SmA*RfG*SmCfAmAmGmAfA | UUAUAGAGCAAGAACACUGUU | RSRSRSRSOOOOOOOOOOO |
| | mCfAmCfUmGfUmU*SmU*SmU | UU | OSS |
| WV-37065 | mU*RfU*SmA*RfU*SmA*RfG*SmA*SfG*RmCfAmAmGmAfA | UUAUAGAGCAAGAACACUGUU | RSRSRSSROOOOOOOOOOO |
| | mCfAmCfUmGfUmU*SmU*SmU | UU | OSS |
| WV-37066 | mU*RfU*SmA*RfU*SmA*RfG*SmA*SfG*SmCfAmAmGmAfA | UUAUAGAGCAAGAACACUGUU | RSRSRSSSOOOOOOOOOOO |
| | mCfAmCfUmGfUmU*SmU*SmU | UU | OSS |
| WV-37067 | mU*RfU*SmA*RfU*SmA*SfG*RmA*RfG*RmCfAmAmGmAfA | UUAUAGAGCAAGAACACUGUU | RSRSSRRROOOOOOOOOOO |
| | mCfAmCfUmGfUmU*SmU*SmU | UU | OSS |
| WV-37068 | mU*RfU*SmA*RfU*SmA*SfG*RmA*RfG*SmCfAmAmGmAfA | UUAUAGAGCAAGAACACUGUU | RSRSSRRSOOOOOOOOOOO |
| | mCfAmCfUmGfUmU*SmU*SmU | UU | OSS |
| WV-37069 | mU*RfU*SmA*RfU*SmA*SfG*RmA*SfG*RmCfAmAmGmAfA | UUAUAGAGCAAGAACACUGUU | RSRSSRSROOOOOOOOOOO |
| | mCfAmCfUmGfUmU*SmU*SmU | UU | OSS |
| WV-37070 | mU*RfU*SmA*RfU*SmA*SfG*RmA*SfG*SmCfAmAmGmAfA | UUAUAGAGCAAGAACACUGUU | RSRSSRSSOOOOOOOOOOO |
| | mCfAmCfUmGfUmU*SmU*SmU | UU | OSS |
| WV-37071 | mU*RfU*SmA*RfU*SmA*SfG*SmA*RfG*RmCfAmAmGmAfA | UUAUAGAGCAAGAACACUGUU | RSRSSSRROOOOOOOOOOO |
| | mCfAmCfUmGfUmU*SmU*SmU | UU | OSS |
| WV-37072 | mU*RfU*SmA*RfU*SmA*SfG*SmA*RfG*SmCfAmAmGmAfA | UUAUAGAGCAAGAACACUGUU | RSRSSSRSOOOOOOOOOOO |
| | mCfAmCfUmGfUmU*SmU*SmU | UU | OSS |
| WV-37073 | mU*RfU*SmA*RfU*SmA*SfG*SmA*SfG*RmCfAmAmGmAfA | UUAUAGAGCAAGAACACUGUU | RSRSSSSROOOOOOOOOOO |
| | mCfAmCfUmGfUmU*SmU*SmU | UU | OSS |
| WV-37074 | mU*RfU*SmA*RfU*SmA*SfG*SmA*SfG*SmCfAmAmGmAfA | UUAUAGAGCAAGAACACUGUU | RSRSSSSSOOOOOOOOOOO |
| | mCfAmCfUmGfUmU*SmU*SmU | UU | OSS |
| WV-37075 | mU*RfU*SmA*SfU*RmA*RfG*RmA*RfG*RmCfAmAmGmAfA | UUAUAGAGCAAGAACACUGUU | RSSRRRRROOOOOOOOOOO |
| | mCfAmCfUmGfUmU*SmU*SmU | UU | OSS |
| WV-37076 | mU*RfU*SmA*SfU*RmA*RfG*RmA*RfG*SmCfAmAmGmAfA | UUAUAGAGCAAGAACACUGUU | RSSRRRRSOOOOOOOOOOO |
| | mCfAmCfUmGfUmU*SmU*SmU | UU | OSS |
| WV-37077 | mU*RfU*SmA*SfU*RmA*RfG*RmA*SfG*RmCfAmAmGmAfA | UUAUAGAGCAAGAACACUGUU | RSSRRRSROOOOOOOOOOO |
| | mCfAmCfUmGfUmU*SmU*SmU | UU | OSS |
| WV-37078 | mU*RfU*SmA*SfU*RmA*RfG*RmA*SfG*SmCfAmAmGmAfA | UUAUAGAGCAAGAACACUGUU | RSSRRRSSOOOOOOOOOOO |
| | mCfAmCfUmGfUmU*SmU*SmU | UU | OSS |
| WV-37079 | mU*RfU*SmA*SfU*RmA*RfG*SmA*RfG*RmCfAmAmGmAfA | UUAUAGAGCAAGAACACUGUU | RSSRRSRROOOOOOOOOOO |
| | mCfAmCfUmGfUmU*SmU*SmU | UU | OSS |
| WV-37080 | mU*RfU*SmA*SfU*RmA*RfG*SmA*RfG*SmCfAmAmGmAfA | UUAUAGAGCAAGAACACUGUU | RSSRRSRSOOOOOOOOOOO |
| | mCfAmCfUmGfUmU*SmU*SmU | UU | OSS |
| WV-37081 | mU*RfU*SmA*SfU*RmA*RfG*SmA*SfG*RmCfAmAmGmAfA | UUAUAGAGCAAGAACACUGUU | RSSRRSSROOOOOOOOOOO |
| | mCfAmCfUmGfUmU*SmU*SmU | UU | OSS |
| WV-37082 | mU*RfU*SmA*SfU*RmA*RfG*SmA*SfG*SmCfAmAmGmAfA | UUAUAGAGCAAGAACACUGUU | RSSRRSSSOOOOOOOOOOO |
| | mCfAmCfUmGfUmU*SmU*SmU | UU | OSS |
| WV-37083 | mU*RfU*SmA*SfU*RmA*SfG*RmA*RfG*RmCfAmAmGmAfA | UUAUAGAGCAAGAACACUGUU | RSSRSRRROOOOOOOOOOO |
| | mCfAmCfUmGfUmU*SmU*SmU | UU | OSS |
| WV-37084 | mU*RfU*SmA*SfU*RmA*SfG*RmA*RfG*SmCfAmAmGmAfA | UUAUAGAGCAAGAACACUGUU | RSSRSRRSOOOOOOOOOOO |
| | mCfAmCfUmGfUmU*SmU*SmU | UU | OSS |
| WV-37085 | mU*RfU*SmA*SfU*RmA*SfG*RmA*SfG*RmCfAmAmGmAfA | UUAUAGAGCAAGAACACUGUU | RSSRSRSROOOOOOOOOOO |
| | mCfAmCfUmGfUmU*SmU*SmU | UU | OSS |
| WV-37086 | mU*RfU*SmA*SfU*RmA*SfG*RmA*SfG*SmCfAmAmGmAfA | UUAUAGAGCAAGAACACUGUU | RSSRSRSSOOOOOOOOOOO |
| | mCfAmCfUmGfUmU*SmU*SmU | UU | OSS |
| WV-37087 | mU*RfU*SmA*SfU*RmA*SfG*SmA*RfG*RmCfAmAmGmAfA | UUAUAGAGCAAGAACACUGUU | RSSRSSRROOOOOOOOOOO |
| | mCfAmCfUmGfUmU*SmU*SmU | UU | OSS |
| WV-37088 | mU*RfU*SmA*SfU*RmA*SfG*SmA*RfG*SmCfAmAmGmAfA | UUAUAGAGCAAGAACACUGUU | RSSRSSRSOOOOOOOOOOO |
| | mCfAmCfUmGfUmU*SmU*SmU | UU | OSS |
| WV-37089 | mU*RfU*SmA*SfU*RmA*SfG*SmA*SfG*RmCfAmAmGmAfA | UUAUAGAGCAAGAACACUGUU | RSSRSSSROOOOOOOOOOO |
| | mCfAmCfUmGfUmU*SmU*SmU | UU | OSS |
| WV-37090 | mU*RfU*SmA*SfU*RmA*SfG*SmA*SfG*SmCfAmAmGmAfA | UUAUAGAGCAAGAACACUGUU | RSSRSSSSOOOOOOOOOOO |
| | mCfAmCfUmGfUmU*SmU*SmU | UU | OSS |
| WV-37091 | mU*RfU*SmA*SfU*SmA*RfG*RmA*RfG*RmCfAmAmGmAfA | UUAUAGAGCAAGAACACUGUU | RSSSRRRROOOOOOOOOOO |
| | mCfAmCfUmGfUmU*SmU*SmU | UU | OSS |
| WV-37092 | mU*RfU*SmA*SfU*SmA*RfG*RmA*RfG*SmCfAmAmGmAfA | UUAUAGAGCAAGAACACUGUU | RSSSRRRSOOOOOOOOOOO |
| | mCfAmCfUmGfUmU*SmU*SmU | UU | OSS |
| WV-37093 | mU*RfU*SmA*SfU*SmA*RfG*RmA*SfG*RmCfAmAmGmAfA | UUAUAGAGCAAGAACACUGUU | RSSSRRSROOOOOOOOOOO |
| | mCfAmCfUmGfUmU*SmU*SmU | UU | OSS |
| WV-37094 | mU*RfU*SmA*SfU*SmA*RfG*RmA*SfG*SmCfAmAmGmAfA | UUAUAGAGCAAGAACACUGUU | RSSSRRSSOOOOOOOOOOO |
| | mCfAmCfUmGfUmU*SmU*SmU | UU | OSS |
| WV-37095 | mU*RfU*SmA*SfU*SmA*RfG*SmA*RfG*RmCfAmAmGmAfA | UUAUAGAGCAAGAACACUGUU | RSSSRSRROOOOOOOOOOO |
| | mCfAmCfUmGfUmU*SmU*SmU | UU | OSS |
| WV-37096 | mU*RfU*SmA*SfU*SmA*RfG*SmA*RfG*SmCfAmAmGmAfA | UUAUAGAGCAAGAACACUGUU | RSSSRSRSOOOOOOOOOOO |
| | mCfAmCfUmGfUmU*SmU*SmU | UU | OSS |
| WV-37097 | mU*RfU*SmA*SfU*SmA*RfG*SmA*SfG*RmCfAmAmGmAfA | UUAUAGAGCAAGAACACUGUU | RSSSRSSROOOOOOOOOOO |
| | mCfAmCfUmGfUmU*SmU*SmU | UU | OSS |
| WV-37098 | mU*RfU*SmA*SfU*SmA*RfG*SmA*SfG*SmCfAmAmGmAfA | UUAUAGAGCAAGAACACUGUU | RSSSRSSSOOOOOOOOOOO |
| | mCfAmCfUmGfUmU*SmU*SmU | UU | OSS |
| WV-37099 | mU*RfU*SmA*SfU*SmA*SfG*RmA*RfG*RmCfAmAmGmAfA | UUAUAGAGCAAGAACACUGUU | RSSSSRRROOOOOOOOOOO |
| | mCfAmCfUmGfUmU*SmU*SmU | UU | OSS |
| WV-37100 | mU*RfU*SmA*SfU*SmA*SfG*RmA*RfG*SmCfAmAmGmAfA | UUAUAGAGCAAGAACACUGUU | RSSSSRRSOOOOOOOOOOO |
| | mCfAmCfUmGfUmU*SmU*SmU | UU | OSS |
| WV-37101 | mU*RfU*SmA*SfU*SmA*SfG*RmA*SfG*RmCfAmAmGmAfA | UUAUAGAGCAAGAACACUGUU | RSSSSRSROOOOOOOOOOO |
| | mCfAmCfUmGfUmU*SmU*SmU | UU | OSS |
| WV-37102 | mU*RfU*SmA*SfU*SmA*SfG*RmA*SfG*SmCfAmAmGmAfA | UUAUAGAGCAAGAACACUGUU | RSSSSRSSOOOOOOOOOOO |
| | mCfAmCfUmGfUmU*SmU*SmU | UU | OSS |
| WV-37103 | mU*RfU*SmA*SfU*SmA*SfG*SmA*RfG*RmCfAmAmGmAfA | UUAUAGAGCAAGAACACUGUU | RSSSSSRROOOOOOOOOOO |
| | mCfAmCfUmGfUmU*SmU*SmU | UU | OSS |
| WV-37104 | mU*RfU*SmA*SfU*SmA*SfG*SmA*RfG*SmCfAmAmGmAfA | UUAUAGAGCAAGAACACUGUU | RSSSSSRSOOOOOOOOOOO |
| | mCfAmCfUmGfUmU*SmU*SmU | UU | OSS |
| WV-37105 | mU*RfU*SmA*SfU*SmA*SfG*SmA*SfG*RmCfAmAmGmAfA | UUAUAGAGCAAGAACACUGUU | RSSSSSSROOOOOOOOOOO |
| | mCfAmCfUmGfUmU*SmU*SmU | UU | OSS |
| WV-37106 | mU*RfU*SmA*SfU*SmA*SfG*SmA*SfG*SmCfAmAmGmAfA | UUAUAGAGCAAGAACACUGUU | RSSSSSSSOOOOOOOOOOO |
| | mCfAmCfUmGfUmU*SmU*SmU | UU | OSS |
| WV-37107 | mU*SfU*RmA*RfU*RmA*RfG*RmA*RfG*RmCfAmAmGmAfA | UUAUAGAGCAAGAACACUGUU | SRRRRRRROOOOOOOOOOO |
| | mCfAmCfUmGfUmU*SmU*SmU | UU | OSS |
| WV-37108 | mU*SfU*RmA*RfU*RmA*RfG*RmA*RfG*SmCfAmAmGmAfA | UUAUAGAGCAAGAACACUGUU | SRRRRRRSOOOOOOOOOOO |
| | mCfAmCfUmGfUmU*SmU*SmU | UU | OSS |
| WV-37109 | mU*SfU*RmA*RfU*RmA*RfG*RmA*SfG*RmCfAmAmGmAfA | UUAUAGAGCAAGAACACUGUU | SRRRRRSROOOOOOOOOOO |
| | mCfAmCfUmGfUmU*SmU*SmU | UU | OSS |
| WV-37110 | mU*SfU*RmA*RfU*RmA*RfG*RmA*SfG*SmCfAmAmGmAfA | UUAUAGAGCAAGAACACUGUU | SRRRRRSSOOOOOOOOOOO |
| | mCfAmCfUmGfUmU*SmU*SmU | UU | OSS |
| WV-37111 | mU*SfU*RmA*RfU*RmA*RfG*SmA*RfG*RmCfAmAmGmAfA | UUAUAGAGCAAGAACACUGUU | SRRRRSRROOOOOOOOOOO |
| | mCfAmCfUmGfUmU*SmU*SmU | UU | OSS |
| WV-37112 | mU*SfU*RmA*RfU*RmA*RfG*SmA*RfG*SmCfAmAmGmAfA | UUAUAGAGCAAGAACACUGUU | SRRRRSRSOOOOOOOOOOO |
| | mCfAmCfUmGfUmU*SmU*SmU | UU | OSS |
| WV-37113 | mU*SfU*RmA*RfU*RmA*RfG*SmA*SfG*RmCfAmAmGmAfA | UUAUAGAGCAAGAACACUGUU | SRRRRSSROOOOOOOOOOO |
| | mCfAmCfUmGfUmU*SmU*SmU | UU | OSS |
| WV-37114 | mU*SfU*RmA*RfU*RmA*RfG*SmA*SfG*SmCfAmAmGmAfA | UUAUAGAGCAAGAACACUGUU | SRRRRSSSOOOOOOOOOOO |
| | mCfAmCfUmGfUmU*SmU*SmU | UU | OSS |
| WV-37115 | mU*SfU*RmA*RfU*RmA*SfG*RmA*RfG*RmCfAmAmGmAfA | UUAUAGAGCAAGAACACUGUU | SRRRSRRROOOOOOOOOOO |
| | mCfAmCfUmGfUmU*SmU*SmU | UU | OSS |
| WV-37116 | mU*SfU*RmA*RfU*RmA*SfG*RmA*RfG*SmCfAmAmGmAfA | UUAUAGAGCAAGAACACUGUU | SRRRSRRSOOOOOOOOOOO |
| | mCfAmCfUmGfUmU*SmU*SmU | UU | OSS |
| WV-37117 | mU*SfU*RmA*RfU*RmA*SfG*RmA*SfG*RmCfAmAmGmAfA | UUAUAGAGCAAGAACACUGUU | SRRRSRSROOOOOOOOOOO |
| | mCfAmCfUmGfUmU*SmU*SmU | UU | OSS |
| WV-37118 | mU*SfU*RmA*RfU*RmA*SfG*RmA*SfG*SmCfAmAmGmAfA | UUAUAGAGCAAGAACACUGUU | SRRRSRSSOOOOOOOOOOO |
| | mCfAmCfUmGfUmU*SmU*SmU | UU | OSS |
| WV-37119 | mU*SfU*RmA*RfU*RmA*SfG*SmA*RfG*RmCfAmAmGmAfA | UUAUAGAGCAAGAACACUGUU | SRRRSSRROOOOOOOOOOO |
| | mCfAmCfUmGfUmU*SmU*SmU | UU | OSS |
| WV-37120 | mU*SfU*RmA*RfU*RmA*SfG*SmA*RfG*SmCfAmAmGmAfA | UUAUAGAGCAAGAACACUGUU | SRRRSSRSOOOOOOOOOOO |
| | mCfAmCfUmGfUmU*SmU*SmU | UU | OSS |
| WV-37121 | mU*SfU*RmA*RfU*RmA*SfG*SmA*SfG*RmCfAmAmGmAfA | UUAUAGAGCAAGAACACUGUU | SRRRSSSROOOOOOOOOOO |
| | mCfAmCfUmGfUmU*SmU*SmU | UU | OSS |
| WV-37122 | mU*SfU*RmA*RfU*RmA*SfG*SmA*SfG*SmCfAmAmGmAfA | UUAUAGAGCAAGAACACUGUU | SRRRSSSSOOOOOOOOOOO |
| | mCfAmCfUmGfUmU*SmU*SmU | UU | OSS |
| WV-37123 | mU*SfU*RmA*RfU*SmA*RfG*RmA*RfG*RmCfAmAmGmAfA | UUAUAGAGCAAGAACACUGUU | SRRSRRRROOOOOOOOOOO |
| | mCfAmCfUmGfUmU*SmU*SmU | UU | OSS |
| WV-37124 | mU*SfU*RmA*RfU*SmA*RfG*RmA*RfG*SmCfAmAmGmAfA | UUAUAGAGCAAGAACACUGUU | SRRSRRRSOOOOOOOOOOO |
| | mCfAmCfUmGfUmU*SmU*SmU | UU | OSS |
| WV-37125 | mU*SfU*RmA*RfU*SmA*RfG*RmA*SfG*RmCfAmAmGmAfA | UUAUAGAGCAAGAACACUGUU | SRRSRRSROOOOOOOOOOO |
| | mCfAmCfUmGfUmU*SmU*SmU | UU | SS |
| WV-37126 | mU*SfU*RmA*RfU*SmA*RfG*RmA*SfG*SmCfAmAmGmAfA | UUAUAGAGCAAGAACACUGUU | SRRSRRSSOOOOOOOOOOO |
| | mCfAmCfUmGfUmU*SmU*SmU | UU | OSS |
| WV-37127 | mU*SfU*RmA*RfU*SmA*RfG*SmA*RfG*RmCfAmAmGmAfA | UUAUAGAGCAAGAACACUGUU | SRRSRSRROOOOOOOOOOO |
| | mCfAmCfUmGfUmU*SmU*SmU | UU | OSS |
| WV-37128 | mU*SfU*RmA*RfU*SmA*RfG*SmA*RfG*SmCfAmAmGmAfA | UUAUAGAGCAAGAACACUGUU | SRRSRSRSOOOOOOOOOOO |
| | mCfAmCfUmGfUmU*SmU*SmU | UU | OSS |
| WV-37129 | mU*SfU*RmA*RfU*SmA*RfG*SmA*SfG*RmCfAmAmGmAfA | UUAUAGAGCAAGAACACUGUU | SRRSRSSROOOOOOOOOOO |
| | mCfAmCfUmGfUmU*SmU*SmU | UU | OSS |
| WV-37130 | mU*SfU*RmA*RfU*SmA*RfG*SmA*SfG*SmCfAmAmGmAfA | UUAUAGAGCAAGAACACUGUU | SRRSRSSSOOOOOOOOOOO |
| | mCfAmCfUmGfUmU*SmU*SmU | UU | OSS |
| WV-37131 | mU*SfU*RmA*RfU*SmA*SfG*RmA*RfG*RmCfAmAmGmAfA | UUAUAGAGCAAGAACACUGUU | SRRSSRRROOOOOOOOOOO |
| | mCfAmCfUmGfUmU*SmU*SmU | UU | OSS |
| WV-37132 | mU*SfU*RmA*RfU*SmA*SfG*RmA*RfG*SmCfAmAmGmAfA | UUAUAGAGCAAGAACACUGUU | SRRSSRRSOOOOOOOOOOO |
| | mCfAmCfUmGfUmU*SmU*SmU | UU | OSS |
| WV-37133 | mU*SfU*RmA*RfU*SmA*SfG*RmA*SfG*RmCfAmAmGmAfA | UUAUAGAGCAAGAACACUGUU | SRRSSRSROOOOOOOOOOO |
| | mCfAmCfUmGfUmU*SmU*SmU | UU | OSS |
| WV-37134 | mU*SfU*RmA*RfU*SmA*SfG*RmA*SfG*SmCfAmAmGmAfA | UUAUAGAGCAAGAACACUGUU | SRRSSRSSOOOOOOOOOOO |
| | mCfAmCfUmGfUmU*SmU*SmU | UU | OSS |
| WV-37135 | mU*SfU*RmA*RfU*SmA*SfG*SmA*RfG*RmCfAmAmGmAfA | UUAUAGAGCAAGAACACUGUU | SRRSSSRROOOOOOOOOOO |
| | mCfAmCfUmGfUmU*SmU*SmU | UU | OSS |
| WV-37136 | mU*SfU*RmA*RfU*SmA*SfG*SmA*RfG*SmCfAmAmGmAfA | UUAUAGAGCAAGAACACUGUU | SRRSSSRSOOOOOOOOOOO |
| | mCfAmCfUmGfUmU*SmU*SmU | UU | OSS |
| WV-37137 | mU*SfU*RmA*RfU*SmA*SfG*SmA*SfG*RmCfAmAmGmAfA | UUAUAGAGCAAGAACACUGUU | SRRSSSSROOOOOOOOOOO |
| | mCfAmCfUmGfUmU*SmU*SmU | UU | OSS |
| WV-37138 | mU*SfU*RmA*RfU*SmA*SfG*SmA*SfG*SmCfAmAmGmAfA | UUAUAGAGCAAGAACACUGUU | SRRSSSSSOOOOOOOOOOO |
| | mCfAmCfUmGfUmU*SmU*SmU | UU | OSS |
| WV-37139 | mU*SfU*RmA*SfU*RmA*RfG*RmA*RfG*RmCfAmAmGmAfA | UUAUAGAGCAAGAACACUGUU | SRSRRRRROOOOOOOOOOO |
| | mCfAmCfUmGfUmU*SmU*SmU | UU | SS |
| WV-37140 | mU*SfU*RmA*SfU*RmA*RfG*RmA*RfG*SmCfAmAmGmAfA | UUAUAGAGCAAGAACACUGUU | SRSRRRRSOOOOOOOOOOO |
| | mCfAmCfUmGfUmU*SmU*SmU | UU | OSS |
| WV-37141 | mU*SfU*RmA*SfU*RmA*RfG*RmA*SfG*RmCfAmAmGmAfA | UUAUAGAGCAAGAACACUGUU | SRSRRRSROOOOOOOOOOO |
| | mCfAmCfUmGfUmU*SmU*SmU | UU | OSS |
| WV-37142 | mU*SfU*RmA*SfU*RmA*RfG*RmA*SfG*SmCfAmAmGmAfA | UUAUAGAGCAAGAACACUGUU | SRSRRRSSOOOOOOOOOOO |
| | mCfAmCfUmGfUmU*SmU*SmU | UU | OSS |
| WV-37143 | mU*SfU*RmA*SfU*RmA*RfG*SmA*RfG*RmCfAmAmGmAfA | UUAUAGAGCAAGAACACUGUU | SRSRRSRROOOOOOOOOOO |
| | mCfAmCfUmGfUmU*SmU*SmU | UU | OSS |
| WV-37144 | mU*SfU*RmA*SfU*RmA*RfG*SmA*RfG*SmCfAmAmGmAfA | UUAUAGAGCAAGAACACUGUU | SRSRRSRSOOOOOOOOOOO |
| | mCfAmCfUmGfUmU*SmU*SmU | UU | OSS |
| WV-37145 | mU*SfU*RmA*SfU*RmA*RfG*SmA*SfG*RmCfAmAmGmAfA | UUAUAGAGCAAGAACACUGUU | SRSRRSSROOOOOOOOOOO |
| | mCfAmCfUmGfUmU*SmU*SmU | UU | OSS |
| WV-37146 | mU*SfU*RmA*SfU*RmA*RfG*SmA*SfG*SmCfAmAmGmAfA | UUAUAGAGCAAGAACACUGUU | SRSRRSSSOOOOOOOOOOO |
| | mCfAmCfUmGfUmU*SmU*SmU | UU | OSS |
| WV-37147 | mU*SfU*RmA*SfU*RmA*SfG*RmA*RfG*RmCfAmAmGmAfA | UUAUAGAGCAAGAACACUGUU | SRSRSRRROOOOOOOOOOO |
| | mCfAmCfUmGfUmU*SmU*SmU | UU | OSS |
| WV-37148 | mU*SfU*RmA*SfU*RmA*SfG*RmA*RfG*SmCfAmAmGmAfA | UUAUAGAGCAAGAACACUGUU | SRSRSRRSOOOOOOOOOOO |
| | mCfAmCfUmGfUmU*SmU*SmU | UU | OSS |
| WV-37149 | mU*SfU*RmA*SfU*RmA*SfG*RmA*SfG*RmCfAmAmGmAfA | UUAUAGAGCAAGAACACUGUU | SRSRSRSROOOOOOOOOOO |
| | mCfAmCfUmGfUmU*SmU*SmU | UU | OSS |
| WV-37150 | mU*SfU*RmA*SfU*RmA*SfG*RmA*SfG*SmCfAmAmGmAfA | UUAUAGAGCAAGAACACUGUU | SRSRSRSSOOOOOOOOOOO |
| | mCfAmCfUmGfUmU*SmU*SmU | UU | OSS |
| WV-37151 | mU*SfU*RmA*SfU*RmA*SfG*SmA*RfG*RmCfAmAmGmAfA | UUAUAGAGCAAGAACACUGUU | SRSRSSRROOOOOOOOOOO |
| | mCfAmCfUmGfUmU*SmU*SmU | UU | OSS |
| WV-37152 | mU*SfU*RmA*SfU*RmA*SfG*SmA*RfG*SmCfAmAmGmAfA | UUAUAGAGCAAGAACACUGUU | SRSRSSRSOOOOOOOOOOO |
| | mCfAmCfUmGfUmU*SmU*SmU | UU | OSS |
| WV-37153 | mU*SfU*RmA*SfU*RmA*SfG*SmA*SfG*RmCfAmAmGmAfA | UUAUAGAGCAAGAACACUGUU | SRSRSSSROOOOOOOOOOO |
| | mCfAmCfUmGfUmU*SmU*SmU | UU | OSS |
| WV-37154 | mU*SfU*RmA*SfU*RmA*SfG*SmA*SfG*SmCfAmAmGmAfA | UUAUAGAGCAAGAACACUGUU | SRSRSSSSOOOOOOOOOOO |
| | mCfAmCfUmGfUmU*SmU*SmU | UU | OSS |
| WV-37155 | mU*SfU*RmA*SfU*SmA*RfG*RmA*RfG*RmCfAmAmGmAfA | UUAUAGAGCAAGAACACUGUU | SRSSRRRROOOOOOOOOOO |
| | mCfAmCfUmGfUmU*SmU*SmU | UU | OSS |
| WV-37156 | mU*SfU*RmA*SfU*SmA*RfG*RmA*RfG*SmCfAmAmGmAfA | UUAUAGAGCAAGAACACUGUU | SRSSRRRSOOOOOOOOOOO |
| | mCfAmCfUmGfUmU*SmU*SmU | UU | OSS |
| WV-37157 | mU*SfU*RmA*SfU*SmA*RfG*RmA*SfG*RmCfAmAmGmAfA | UUAUAGAGCAAGAACACUGUU | SRSSRRSROOOOOOOOOOO |
| | mCfAmCfUmGfUmU*SmU*SmU | UU | OSS |
| WV-37158 | mU*SfU*RmA*SfU*SmA*RfG*RmA*SfG*SmCfAmAmGmAfA | UUAUAGAGCAAGAACACUGUU | SRSSRRSSOOOOOOOOOOO |
| | mCfAmCfUmGfUmU*SmU*SmU | UU | OSS |
| WV-37159 | mU*SfU*RmA*SfU*SmA*RfG*SmA*RfG*RmCfAmAmGmAfA | UUAUAGAGCAAGAACACUGUU | SRSSRSRROOOOOOOOOOO |
| | mCfAmCfUmGfUmU*SmU*SmU | UU | OSS |
| WV-37160 | mU*SfU*RmA*SfU*SmA*RfG*SmA*RfG*SmCfAmAmGmAfA | UUAUAGAGCAAGAACACUGUU | SRSSRSRSOOOOOOOOOOO |
| | mCfAmCfUmGfUmU*SmU*SmU | UU | OSS |
| WV-37161 | mU*SfU*RmA*SfU*SmA*RfG*SmA*SfG*RmCfAmAmGmAfA | UUAUAGAGCAAGAACACUGUU | SRSSRSSROOOOOOOOOOO |
| | mCfAmCfUmGfUmU*SmU*SmU | UU | OSS |
| WV-37162 | mU*SfU*RmA*SfU*SmA*RfG*SmA*SfG*SmCfAmAmGmAfA | UUAUAGAGCAAGAACACUGUU | SRSSRSSSOOOOOOOOOOO |
| | mCfAmCfUmGfUmU*SmU*SmU | UU | OSS |
| WV-37163 | mU*SfU*RmA*SfU*SmA*SfG*RmA*RfG*RmCfAmAmGmAfA | UUAUAGAGCAAGAACACUGUU | SRSSSRRROOOOOOOOOOO |
| | mCfAmCfUmGfUmU*SmU*SmU | UU | OSS |
| WV-37164 | mU*SfU*RmA*SfU*SmA*SfG*RmA*RfG*SmCfAmAmGmAfA | UUAUAGAGCAAGAACACUGUU | SRSSSRRSOOOOOOOOOOO |
| | mCfAmCfUmGfUmU*SmU*SmU | UU | OSS |
| WV-37165 | mU*SfU*RmA*SfU*SmA*SfG*RmA*SfG*RmCfAmAmGmAfA | UUAUAGAGCAAGAACACUGUU | SRSSSRSROOOOOOOOOOO |
| | mCfAmCfUmGfUmU*SmU*SmU | UU | OSS |
| WV-37166 | mU*SfU*RmA*SfU*SmA*SfG*RmA*SfG*SmCfAmAmGmAfA | UUAUAGAGCAAGAACACUGUU | SRSSSRSSOOOOOOOOOOO |
| | mCfAmCfUmGfUmU*SmU*SmU | UU | OSS |
| WV-37167 | mU*SfU*RmA*SfU*SmA*SfG*SmA*RfG*RmCfAmAmGmAfA | UUAUAGAGCAAGAACACUGUU | SRSSSSRROOOOOOOOOOO |
| | mCfAmCfUmGfUmU*SmU*SmU | UU | OSS |
| WV-37168 | mU*SfU*RmA*SfU*SmA*SfG*SmA*RfG*SmCfAmAmGmAfA | UUAUAGAGCAAGAACACUGUU | SRSSSSRSOOOOOOOOOOO |
| | mCfAmCfUmGfUmU*SmU*SmU | UU | OSS |
| WV-37169 | mU*SfU*RmA*SfU*SmA*SfG*SmA*SfG*RmCfAmAmGmAfA | UUAUAGAGCAAGAACACUGUU | SRSSSSSROOOOOOOOOOO |
| | mCfAmCfUmGfUmU*SmU*SmU | UU | OSS |
| WV-37170 | mU*SfU*RmA*SfU*SmA*SfG*SmA*SfG*SmCfAmAmGmAfA | UUAUAGAGCAAGAACACUGUU | SRSSSSSSOOOOOOOOOOO |
| | mCfAmCfUmGfUmU*SmU*SmU | UU | OSS |
| WV-37171 | mU*SfU*SmA*RfU*RmA*RfG*RmA*RfG*RmCfAmAmGmAfA | UUAUAGAGCAAGAACACUGUU | SSRRRRRROOOOOOOOOOO |
| | mCfAmCfUmGfUmU*SmU*SmU | UU | OSS |
| WV-37172 | mU*SfU*SmA*RfU*RmA*RfG*RmA*RfG*SmCfAmAmGmAfA | UUAUAGAGCAAGAACACUGUU | SSRRRRRSOOOOOOOOOOO |
| | mCfAmCfUmGfUmU*SmU*SmU | UU | OSS |
| WV-37173 | mU*SfU*SmA*RfU*RmA*RfG*RmA*SfG*RmCfAmAmGmAfA | UUAUAGAGCAAGAACACUGUU | SSRRRRSROOOOOOOOOOO |
| | mCfAmCfUmGfUmU*SmU*SmU | UU | OSS |
| WV-37174 | mU*SfU*SmA*RfU*RmA*RfG*RmA*SfG*SmCfAmAmGmAfA | UUAUAGAGCAAGAACACUGUU | SSRRRRSSOOOOOOOOOOO |
| | mCfAmCfUmGfUmU*SmU*SmU | UU | OSS |
| WV-37175 | mU*SfU*SmA*RfU*RmA*RfG*SmA*RfG*RmCfAmAmGmAfA | UUAUAGAGCAAGAACACUGUU | SSRRRSRROOOOOOOOOOO |
| | mCfAmCfUmGfUmU*SmU*SmU | UU | OSS |
| WV-37176 | mU*SfU*SmA*RfU*RmA*RfG*SmA*RfG*SmCfAmAmGmAfA | UUAUAGAGCAAGAACACUGUU | SSRRRSRSOOOOOOOOOOO |
| | mCfAmCfUmGfUmU*SmU*SmU | UU | OSS |
| WV-37177 | mU*SfU*SmA*RfU*RmA*RfG*SmA*SfG*RmCfAmAmGmAfA | UUAUAGAGCAAGAACACUGUU | SSRRRSSROOOOOOOOOOO |
| | mCfAmCfUmGfUmU*SmU*SmU | UU | OSS |
| WV-37178 | mU*SfU*SmA*RfU*RmA*RfG*SmA*SfG*SmCfAmAmGmAfA | UUAUAGAGCAAGAACACUGUU | SSRRRSSSOOOOOOOOOOO |
| | mCfAmCfUmGfUmU*SmU*SmU | UU | OSS |
| WV-37179 | mU*SfU*SmA*RfU*RmA*SfG*RmA*RfG*RmCfAmAmGmAfA | UUAUAGAGCAAGAACACUGUU | SSRRSRRROOOOOOOOOOO |
| | mCfAmCfUmGfUmU*SmU*SmU | UU | OSS |
| WV-37180 | mU*SfU*SmA*RfU*RmA*SfG*RmA*RfG*SmCfAmAmGmAfA | UUAUAGAGCAAGAACACUGUU | SSRRSRRSOOOOOOOOOOO |
| | mCfAmCfUmGfUmU*SmU*SmU | UU | OSS |
| WV-37181 | mU*SfU*SmA*RfU*RmA*SfG*RmA*SfG*RmCfAmAmGmAfA | UUAUAGAGCAAGAACACUGUU | SSRRSRSROOOOOOOOOOO |
| | mCfAmCfUmGfUmU*SmU*SmU | UU | OSS |
| WV-37182 | mU*SfU*SmA*RfU*RmA*SfG*RmA*SfG*SmCfAmAmGmAfA | UUAUAGAGCAAGAACACUGUU | SSRRSRSSOOOOOOOOOOO |
| | mCfAmCfUmGfUmU*SmU*SmU | UU | OSS |
| WV-37183 | mU*SfU*SmA*RfU*RmA*SfG*SmA*RfG*RmCfAmAmGmAfA | UUAUAGAGCAAGAACACUGUU | SSRRSSRROOOOOOOOOOO |
| | mCfAmCfUmGfUmU*SmU*SmU | UU | OSS |
| WV-37184 | mU*SfU*SmA*RfU*RmA*SfG*SmA*RfG*SmCfAmAmGmAfA | UUAUAGAGCAAGAACACUGUU | SSRRSSRSOOOOOOOOOOO |
| | mCfAmCfUmGfUmU*SmU*SmU | UU | OSS |
| WV-37185 | mU*SfU*SmA*RfU*RmA*SfG*SmA*SfG*RmCfAmAmGmAfA | UUAUAGAGCAAGAACACUGUU | SSRRSSSROOOOOOOOOOO |
| | mCfAmCfUmGfUmU*SmU*SmU | UU | OSS |
| WV-37186 | mU*SfU*SmA*RfU*RmA*SfG*SmA*SfG*SmCfAmAmGmAfA | UUAUAGAGCAAGAACACUGUU | SSRRSSSSOOOOOOOOOOO |
| | mCfAmCfUmGfUmU*SmU*SmU | UU | OSS |
| WV-37187 | mU*SfU*SmA*RfU*SmA*RfG*RmA*RfG*RmCfAmAmGmAfA | UUAUAGAGCAAGAACACUGUU | SSRSRRRROOOOOOOOOOO |
| | mCfAmCfUmGfUmU*SmU*SmU | UU | OSS |
| WV-37188 | mU*SfU*SmA*RfU*SmA*RfG*RmA*RfG*SmCfAmAmGmAfA | UUAUAGAGCAAGAACACUGUU | SSRSRRRSOOOOOOOOOOO |
| | mCfAmCfUmGfUmU*SmU*SmU | UU | OSS |
| WV-37189 | mU*SfU*SmA*RfU*SmA*RfG*RmA*SfG*RmCfAmAmGmAfA | UUAUAGAGCAAGAACACUGUU | SSRSRRSROOOOOOOOOOO |
| | mCfAmCfUmGfUmU*SmU*SmU | UU | OSS |
| WV-37190 | mU*SfU*SmA*RfU*SmA*RfG*RmA*SfG*SmCfAmAmGmAfA | UUAUAGAGCAAGAACACUGUU | SSRSRRSSOOOOOOOOOOO |
| | mCfAmCfUmGfUmU*SmU*SmU | UU | OSS |
| WV-37191 | mU*SfU*SmA*RfU*SmA*RfG*SmA*RfG*RmCfAmAmGmAfA | UUAUAGAGCAAGAACACUGUU | SSRSRSRROOOOOOOOOOO |
| | mCfAmCfUmGfUmU*SmU*SmU | UU | OSS |
| WV-37192 | mU*SfU*SmA*RfU*SmA*RfG*SmA*RfG*SmCfAmAmGmAfA | UUAUAGAGCAAGAACACUGUU | SSRSRSRSOOOOOOOOOOO |
| | mCfAmCfUmGfUmU*SmU*SmU | UU | OSS |
| WV-37193 | mU*SfU*SmA*RfU*SmA*RfG*SmA*SfG*RmCfAmAmGmAfA | UUAUAGAGCAAGAACACUGUU | SSRSRSSROOOOOOOOOOO |
| | mCfAmCfUmGfUmU*SmU*SmU | UU | OSS |
| WV-37194 | mU*SfU*SmA*RfU*SmA*RfG*SmA*SfG*SmCfAmAmGmAfA | UUAUAGAGCAAGAACACUGUU | SSRSRSSSOOOOOOOOOOO |
| | mCfAmCfUmGfUmU*SmU*SmU | UU | OSS |
| WV-37195 | mU*SfU*SmA*RfU*SmA*SfG*RmA*RfG*RmCfAmAmGmAfA | UUAUAGAGCAAGAACACUGUU | SSRSSRRROOOOOOOOOOO |
| | mCfAmCfUmGfUmU*SmU*SmU | UU | OSS |
| WV-37196 | mU*SfU*SmA*RfU*SmA*SfG*RmA*RfG*SmCfAmAmGmAfA | UUAUAGAGCAAGAACACUGUU | SSRSSRRSOOOOOOOOOOO |
| | mCfAmCfUmGfUmU*SmU*SmU | UU | OSS |
| WV-37197 | mU*SfU*SmA*RfU*SmA*SfG*RmA*SfG*RmCfAmAmGmAfA | UUAUAGAGCAAGAACACUGUU | SSRSSRSROOOOOOOOOOO |
| | mCfAmCfUmGfUmU*SmU*SmU | UU | OSS |
| WV-37198 | mU*SfU*SmA*RfU*SmA*SfG*RmA*SfG*SmCfAmAmGmAfA | UUAUAGAGCAAGAACACUGUU | SSRSSRSSOOOOOOOOOOO |
| | mCfAmCfUmGfUmU*SmU*SmU | UU | OSS |
| WV-37199 | mU*SfU*SmA*RfU*SmA*SfG*SmA*RfG*RmCfAmAmGmAfA | UUAUAGAGCAAGAACACUGUU | SSRSSSRROOOOOOOOOOO |
| | mCfAmCfUmGfUmU*SmU*SmU | UU | OSS |
| WV-37200 | mU*SfU*SmA*RfU*SmA*SfG*SmA*RfG*SmCfAmAmGmAfA | UUAUAGAGCAAGAACACUGUU | SSRSSSRSOOOOOOOOOOO |
| | mCfAmCfUmGfUmU*SmU*SmU | UU | OSS |
| WV-37201 | mU*SfU*SmA*RfU*SmA*SfG*SmA*SfG*RmCfAmAmGmAfA | UUAUAGAGCAAGAACACUGUU | SSRSSSSROOOOOOOOOOO |
| | mCfAmCfUmGfUmU*SmU*SmU | UU | OSS |
| WV-37202 | mU*SfU*SmA*RfU*SmA*SfG*SmA*SfG*SmCfAmAmGmAfA | UUAUAGAGCAAGAACACUGUU | SSRSSSSSOOOOOOOOOOO |
| | mCfAmCfUmGfUmU*SmU*SmU | UU | OSS |
| WV-37203 | mU*SfU*SmA*SfU*RmA*RfG*RmA*RfG*RmCfAmAmGmAfA | UUAUAGAGCAAGAACACUGUU | SSSRRRRROOOOOOOOOOO |
| | mCfAmCfUmGfUmU*SmU*SmU | UU | OSS |
| WV-37204 | mU*SfU*SmA*SfU*RmA*RfG*RmA*RfG*SmCfAmAmGmAfA | UUAUAGAGCAAGAACACUGUU | SSSRRRRSOOOOOOOOOOO |
| | mCfAmCfUmGfUmU*SmU*SmU | UU | OSS |
| WV-37205 | mU*SfU*SmA*SfU*RmA*RfG*RmA*SfG*RmCfAmAmGmAfA | UUAUAGAGCAAGAACACUGUU | SSSRRRSROOOOOOOOOOO |
| | mCfAmCfUmGfUmU*SmU*SmU | UU | OSS |
| WV-37206 | mU*SfU*SmA*SfU*RmA*RfG*RmA*SfG*SmCfAmAmGmAfA | UUAUAGAGCAAGAACACUGUU | SSSRRRSSOOOOOOOOOOO |
| | mCfAmCfUmGfUmU*SmU*SmU | UU | OSS |
| WV-37207 | mU*SfU*SmA*SfU*RmA*RfG*SmA*RfG*RmCfAmAmGmAfA | UUAUAGAGCAAGAACACUGUU | SSSRRSRROOOOOOOOOOO |
| | mCfAmCfUmGfUmU*SmU*SmU | UU | OSS |
| WV-37208 | mU*SfU*SmA*SfU*RmA*RfG*SmA*RfG*SmCfAmAmGmAfA | UUAUAGAGCAAGAACACUGUU | SSSRRSRSOOOOOOOOOOO |
| | mCfAmCfUmGfUmU*SmU*SmU | UU | OSS |
| WV-37209 | mU*SfU*SmA*SfU*RmA*RfG*SmA*SfG*RmCfAmAmGmAfA | UUAUAGAGCAAGAACACUGUU | SSSRRSSROOOOOOOOOOO |
| | mCfAmCfUmGfUmU*SmU*SmU | UU | OSS |
| WV-37210 | mU*SfU*SmA*SfU*RmA*RfG*SmA*SfG*SmCfAmAmGmAfA | UUAUAGAGCAAGAACACUGUU | SSSRRSSSOOOOOOOOOOO |
| | mCfAmCfUmGfUmU*SmU*SmU | UU | OSS |
| WV-37211 | mU*SfU*SmA*SfU*RmA*SfG*RmA*RfG*RmCfAmAmGmAfA | UUAUAGAGCAAGAACACUGUU | SSSRSRRROOOOOOOOOOO |
| | mCfAmCfUmGfUmU*SmU*SmU | UU | OSS |
| WV-37212 | mU*SfU*SmA*SfU*RmA*SfG*RmA*RfG*SmCfAmAmGmAfA | UUAUAGAGCAAGAACACUGUU | SSSRSRRSOOOOOOOOOOO |
| | mCfAmCfUmGfUmU*SmU*SmU | UU | OSS |
| WV-37213 | mU*SfU*SmA*SfU*RmA*SfG*RmA*SfG*RmCfAmAmGmAfA | UUAUAGAGCAAGAACACUGUU | SSSRSRSROOOOOOOOOOO |
| | mCfAmCfUmGfUmU*SmU*SmU | UU | OSS |
| WV-37214 | mU*SfU*SmA*SfU*RmA*SfG*RmA*SfG*SmCfAmAmGmAfA | UUAUAGAGCAAGAACACUGUU | SSSRSRSSOOOOOOOOOOO |
| | mCfAmCfUmGfUmU*SmU*SmU | UU | OSS |
| WV-37215 | mU*SfU*SmA*SfU*RmA*SfG*SmA*RfG*RmCfAmAmGmAfA | UUAUAGAGCAAGAACACUGUU | SSSRSSRROOOOOOOOOOO |
| | mCfAmCfUmGfUmU*SmU*SmU | UU | OSS |
| WV-37216 | mU*SfU*SmA*SfU*RmA*SfG*SmA*RfG*SmCfAmAmGmAfA | UUAUAGAGCAAGAACACUGUU | SSSRSSRSOOOOOOOOOOO |
| | mCfAmCfUmGfUmU*SmU*SmU | UU | OSS |
| WV-37217 | mU*SfU*SmA*SfU*RmA*SfG*SmA*SfG*RmCfAmAmGmAfA | UUAUAGAGCAAGAACACUGUU | SSSRSSSROOOOOOOOOOO |
| | mCfAmCfUmGfUmU*SmU*SmU | UU | OSS |
| WV-37218 | mU*SfU*SmA*SfU*RmA*SfG*SmA*SfG*SmCfAmAmGmAfA | UUAUAGAGCAAGAACACUGUU | SSSRSSSSOOOOOOOOOOO |
| | mCfAmCfUmGfUmU*SmU*SmU | UU | OSS |
| WV-37219 | mU*SfU*SmA*SfU*SmA*RfG*RmA*RfG*RmCfAmAmGmAfA | UUAUAGAGCAAGAACACUGUU | SSSSRRRROOOOOOOOOOO |
| | mCfAmCfUmGfUmU*SmU*SmU | UU | OSS |
| WV-37220 | mU*SfU*SmA*SfU*SmA*RfG*RmA*RfG*SmCfAmAmGmAfA | UUAUAGAGCAAGAACACUGUU | SSSSRRRSOOOOOOOOOOO |
| | mCfAmCfUmGfUmU*SmU*SmU | UU | OSS |
| WV-37221 | mU*SfU*SmA*SfU*SmA*RfG*RmA*SfG*RmCfAmAmGmAfA | UUAUAGAGCAAGAACACUGUU | SSSSRRSROOOOOOOOOOO |
| | mCfAmCfUmGfUmU*SmU*SmU | UU | OSS |
| WV-37222 | mU*SfU*SmA*SfU*SmA*RfG*RmA*SfG*SmCfAmAmGmAfA | UUAUAGAGCAAGAACACUGUU | SSSSRRSSOOOOOOOOOOO |
| | mCfAmCfUmGfUmU*SmU*SmU | UU | OSS |
| WV-37223 | mU*SfU*SmA*SfU*SmA*RfG*SmA*RfG*RmCfAmAmGmAfA | UUAUAGAGCAAGAACACUGUU | SSSSRSRROOOOOOOOOOO |
| | mCfAmCfUmGfUmU*SmU*SmU | UU | OSS |
| WV-37224 | mU*SfU*SmA*SfU*SmA*RfG*SmA*RfG*SmCfAmAmGmAfA | UUAUAGAGCAAGAACACUGUU | SSSSRSRSOOOOOOOOOOO |
| | mCfAmCfUmGfUmU*SmU*SmU | UU | OSS |
| WV-37225 | mU*SfU*SmA*SfU*SmA*RfG*SmA*SfG*RmCfAmAmGmAfA | UUAUAGAGCAAGAACACUGUU | SSSSRSSROOOOOOOOOOO |
| | mCfAmCfUmGfUmU*SmU*SmU | UU | OSS |
| WV-37226 | mU*SfU*SmA*SfU*SmA*RfG*SmA*SfG*SmCfAmAmGmAfA | UUAUAGAGCAAGAACACUGUU | SSSSRSSSOOOOOOOOOOO |
| | mCfAmCfUmGfUmU*SmU*SmU | UU | OSS |
| WV-37227 | mU*SfU*SmA*SfU*SmA*SfG*RmA*RfG*RmCfAmAmGmAfA | UUAUAGAGCAAGAACACUGUU | SSSSSRRROOOOOOOOOOO |
| | mCfAmCfUmGfUmU*SmU*SmU | UU | OSS |
| WV-37228 | mU*SfU*SmA*SfU*SmA*SfG*RmA*RfG*SmCfAmAmGmAfA | UUAUAGAGCAAGAACACUGUU | SSSSSRRSOOOOOOOOOOO |
| | mCfAmCfUmGfUmU*SmU*SmU | UU | OSS |
| WV-37229 | mU*SfU*SmA*SfU*SmA*SfG*RmA*SfG*RmCfAmAmGmAfA | UUAUAGAGCAAGAACACUGUU | SSSSSRSROOOOOOOOOOO |
| | mCfAmCfUmGfUmU*SmU*SmU | UU | OSS |
| WV-37230 | mU*SfU*SmA*SfU*SmA*SfG*RmA*SfG*SmCfAmAmGmAfA | UUAUAGAGCAAGAACACUGUU | SSSSSRSSOOOOOOOOOOO |
| | mCfAmCfUmGfUmU*SmU*SmU | UU | OSS |
| WV-37231 | mU*SfU*SmA*SfU*SmA*SfG*SmA*RfG*RmCfAmAmGmAfA | UUAUAGAGCAAGAACACUGUU | SSSSSSRROOOOOOOOOOO |
| | mCfAmCfUmGfUmU*SmU*SmU | UU | OSS |
| WV-37232 | mU*SfU*SmA*SfU*SmA*SfG*SmA*RfG*SmCfAmAmGmAfA | UUAUAGAGCAAGAACACUGUU | SSSSSSRSOOOOOOOOOOO |
| | mCfAmCfUmGfUmU*SmU*SmU | UU | OSS |
| WV-37233 | mU*SfU*SmA*SfU*SmA*SfG*SmA*SfG*RmCfAmAmGmAfA | UUAUAGAGCAAGAACACUGUU | SSSSSSSROOOOOOOOOOO |
| | mCfAmCfUmGfUmU*SmU*SmU | UU | OSS |
| WV-37234 | mU*SfU*SmA*SfU*SmA*SfG*SmA*SfG*SmCfAmAmGmAfA | UUAUAGAGCAAGAACACUGUU | SSSSSSSSOOOOOOOOOOO |
| | mCfAmCfUmGfUmU*SmU*SmU | UU | OSS |
| WV-37235 | mU*RfU*RmA*RfU*RmA*RfG*RmA*RfG*RmCfAmAmGmAfA | UUAUAGAGCAAGAACACUGUU | RRRRRRRROOOOOOOOOOO |
| | mCfAmCfUmGfUmU*SmU*SmU | UU | OSS |
| WV-37236 | mU*fU*mA*fU*mA*fG*mA*fG*mCfAmAmGmAfAmCfAmCfU | UUAUAGAGCAAGAACACUGUU | XXXXXXXXOOOOOOOOOOO |
| | mGfUmU*mU*mU | UU | OXX |
| WV-37236 | mU*fU*mA*fU*mA*fG*mA*fG*mCfAmAmGmAfAmCfAmCfU | UUAUAGAGCAAGAACACUGUU | XXXXXXXXOOOOOOOOOOO |
| | mGfUmU*mU*mU | UU | OXX |
| WV-38082 | mUfUmAfUmAfGmAfGmCfAmAmGmAfAmCfAmCfUmGfUmU*m | UUAUAGAGCAAGAACACUGUU | OOOOOOOOOOOOOOOOOOO |
| | U*mU | UU | OXX |
| WV-38083 | mUfUmAfUmAfGmAfGmCfAmAmGmAfAmCfAmCfUmGfUmU*S | UUAUAGAGCAAGAACACUGUU | OOOOOOOOOOOOOOOOOOO |
| | mU*SmU | UU | OSS |
| WV-38087 | mU*RfUmAfUmAfGmAfGmCfAmAmGmAfAmCfAmCfUmGfUmU | UUAUAGAGCAAGAACACUGUU | ROOOOOOOOOOOOOOOOOO |
| | *SmU*SmU | UU | OSS |
| WV-38088 | mUfU*RmAfUmAfGmAfGmCfAmAmGmAfAmCfAmCfUmGfUmU | UUAUAGAGCAAGAACACUGUU | OROOOOOOOOOOOOOOOOO |
| | *SmU*SmU | UU | OSS |
| WV-38089 | mUfUmA*RfUmAfGmAfGmCfAmAmGmAfAmCfAmCfUmGfUmU | UUAUAGAGCAAGAACACUGUU | OOROOOOOOOOOOOOOOOO |
| | *SmU*SmU | UU | OSS |
| WV-38090 | mUfUmAfU*RmAfGmAfGmCfAmAmGmAfAmCfAmCfUmGfUmU | UUAUAGAGCAAGAACACUGUU | OOOROOOOOOOOOOOOOOO |
| | *SmU*SmU | UU | OSS |
| WV-38091 | mUfUmAfUmA*RfGmAfGmCfAmAmGmAfAmCfAmCfUmGfUmU | UUAUAGAGCAAGAACACUGUU | OOOOROOOOOOOOOOOOOO |
| | *SmU*SmU | UU | OSS |
| WV-38092 | mUfUmAfUmAfG*RmAfGmCfAmAmGmAfAmCfAmCfUmGfUmU | UUAUAGAGCAAGAACACUGUU | OOOOOROOOOOOOOOOOOO |
| | *SmU*SmU | UU | OSS |
| WV-38093 | mUfUmAfUmAfGmA*RfGmCfAmAmGmAfAmCfAmCfUmGfUmU | UUAUAGAGCAAGAACACUGUU | OOOOOOROOOOOOOOOOOO |
| | *SmU*SmU | UU | OSS |
| WV-38094 | mUfUmAfUmAfGmAfG*RmCfAmAmGmAfAmCfAmCfUmGfUmU | UUAUAGAGCAAGAACACUGUU | OOOOOOOROOOOOOOOOOO |
| | *SmU*SmU | UU | OSS |
| WV-38095 | mUfUmAfUmAfGmAfGmC*RfAmAmGmAfAmCfAmCfUmGfUmU | UUAUAGAGCAAGAACACUGUU | OOOOOOOOROOOOOOOOOO |
| | *SmU*SmU | UU | OSS |
| WV-38096 | mUfUmAfUmAfGmAfGmCfA*RmAmGmAfAmCfAmCfUmGfUmU | UUAUAGAGCAAGAACACUGUU | OOOOOOOOOROOOOOOOOO |
| | *SmU*SmU | UU | OSS |
| WV-38097 | mUfUmAfUmAfGmAfGmCfAmA*RmGmAfAmCfAmCfUmGfUmU | UUAUAGAGCAAGAACACUGUU | OOOOOOOOOOROOOOOOOO |
| | *SmU*SmU | UU | OSS |
| WV-38098 | mUfUmAfUmAfGmAfGmCfAmAmG*RmAfAmCfAmCfUmGfUmU | UUAUAGAGCAAGAACACUGUU | OOOOOOOOOOOROOOOOOO |
| | *SmU*SmU | UU | OSS |
| WV-38099 | mUfUmAfUmAfGmAfGmCfAmAmGmA*RfAmCfAmCfUmGfUmU | UUAUAGAGCAAGAACACUGUU | OOOOOOOOOOOOROOOOOO |
| | *SmU*SmU | UU | OSS |
| WV-38100 | mUfUmAfUmAfGmAfGmCfAmAmGmAfA*RmCfAmCfUmGfUmU | UUAUAGAGCAAGAACACUGUU | OOOOOOOOOOOOOROOOOO |
| | *SmU*SmU | UU | OSS |
| WV-38101 | mUfUmAfUmAfGmAfGmCfAmAmGmAfAmC*RfAmCfUmGfUmU | UUAUAGAGCAAGAACACUGUU | OOOOOOOOOOOOOOROOOO |
| | *SmU*SmU | UU | OSS |
| WV-38102 | mUfUmAfUmAfGmAfGmCfAmAmGmAfAmCfA*RmCfUmGfUmU | UUAUAGAGCAAGAACACUGUU | OOOOOOOOOOOOOOOROOO |
| | *SmU*SmU | UU | OSS |
| WV-38103 | mUfUmAfUmAfGmAfGmCfAmAmGmAfAmCfAmC*RfUmGfUmU | UUAUAGAGCAAGAACACUGUU | OOOOOOOOOOOOOOOOROO |
| | *SmU*SmU | UU | OSS |
| WV-38104 | mUfUmAfUmAfGmAfGmCfAmAmGmAfAmCfAmCfU*RmGfUmU | UUAUAGAGCAAGAACACUGUU | OOOOOOOOOOOOOOOOORO |
| | *SmU*SmU | UU | OSS |
| WV-38105 | mUfUmAfUmAfGmAfGmCfAmAmGmAfAmCfAmCfUmG*RfUmU | UUAUAGAGCAAGAACACUGUU | OOOOOOOOOOOOOOOOOOR |
| | *SmU*SmU | UU | OSS |
| WV-38106 | mUfUmAfUmAfGmAfGmCfAmAmGmAfAmCfAmCfUmGfU*RmU | UUAUAGAGCAAGAACACUGUU | OOOOOOOOOOOOOOOOOOO |
| | *SmU*SmU | UU | RSS |
| WV-38107 | mU*SfUmAfUmAfGmAfGmCfAmAmGmAfAmCfAmCfUmGfUmU | UUAUAGAGCAAGAACACUGUU | SOOOOOOOOOOOOOOOOOO |
| | *SmU*SmU | UU | OSS |
| WV-38108 | mUfU*SmAfUmAfGmAfGmCfAmAmGmAfAmCfAmCfUmGfUmU | UUAUAGAGCAAGAACACUGUU | OSOOOOOOOOOOOOOOOOO |
| | *SmU*SmU | UU | OSS |
| WV-38109 | mUfUmA*SfUmAfGmAfGmCfAmAmGmAfAmCfAmCfUmGfUmU | UUAUAGAGCAAGAACACUGUU | OOSOOOOOOOOOOOOOOOO |
| | *SmU*SmU | UU | OSS |
| WV-38110 | mUfUmAfU*SmAfGmAfGmCfAmAmGmAfAmCfAmCfUmGfUmU | UUAUAGAGCAAGAACACUGUU | OOOSOOOOOOOOOOOOOOO |
| | *SmU*SmU | UU | OSS |
| WV-38111 | mUfUmAfUmA*SfGmAfGmCfAmAmGmAfAmCfAmCfUmGfUmU | UUAUAGAGCAAGAACACUGUU | OOOOSOOOOOOOOOOOOOO |
| | *SmU*SmU | UU | OSS |
| WV-38112 | mUfUmAfUmAfG*SmAfGmCfAmAmGmAfAmCfAmCfUmGfUmU | UUAUAGAGCAAGAACACUGUU | OOOOOSOOOOOOOOOOOOO |
| | *SmU*SmU | UU | OSS |
| WV-38113 | mUfUmAfUmAfGmA*SfGmCfAmAmGmAfAmCfAmCfUmGfUmU | UUAUAGAGCAAGAACACUGUU | OOOOOOSOOOOOOOOOOOO |
| | *SmU*SmU | UU | OSS |
| WV-38114 | mUfUmAfUmAfGmAfG*SmCfAmAmGmAfAmCfAmCfUmGfUmU | UUAUAGAGCAAGAACACUGUU | OOOOOOOSOOOOOOOOOOO |
| | *SmU*SmU | UU | OSS |
| WV-38115 | mUfUmAfUmAfGmAfGmC*SfAmAmGmAfAmCfAmCfUmGfUmU | UUAUAGAGCAAGAACACUGUU | OOOOOOOOSOOOOOOOOOO |
| | *SmU*SmU | UU | OSS |
| WV-38116 | mUfUmAfUmAfGmAfGmCfA*SmAmGmAfAmCfAmCfUmGfUmU | UUAUAGAGCAAGAACACUGUU | OOOOOOOOOSOOOOOOOOO |
| | *SmU*SmU | UU | SS |
| WV-38117 | mUfUmAfUmAfGmAfGmCfAmA*SmGmAfAmCfAmCfUmGfUmU | UUAUAGAGCAAGAACACUGUU | OOOOOOOOOOOOOOOOOOO |
| | *SmU*SmU | UU | SS |
| WV-38118 | mUfUmAfUmAfGmAfGmCfAmAmG*SmAfAmCfAmCfUmGfUmU | UUAUAGAGCAAGAACACUGUU | OOOOOOOOOOOSOOOOOOO |
| | *SmU*SmU | UU | OSS |
| WV-38119 | mUfUmAfUmAfGmAfGmCfAmAmGmA*SfAmCfAmCfUmGfUmU | UUAUAGAGCAAGAACACUGUU | OOOOOOOOOOOOOOOOOOO |
| | *SmU*SmU | UU | SS |
| WV-38120 | mUfUmAfUmAfGmAfGmCfAmAmGmAfA*SmCfAmCfUmGfUmU | UUAUAGAGCAAGAACACUGUU | OOOOOOOOOOOOOOOOOOO |
| | *SmU*SmU | UU | SS |
| WV-38121 | mUfUmAfUmAfGmAfGmCfAmAmGmAfAmC*SfAmCfUmGfUmU | UUAUAGAGCAAGAACACUGUU | OOOOOOOOOOOOOOSOOOO |
| | *SmU*SmU | UU | OSS |
| WV-38122 | mUfUmAfUmAfGmAfGmCfAmAmGmAfAmCfA*SmCfUmGfUmU | UUAUAGAGCAAGAACACUGUU | OOOOOOOOOOOOOOOSOOO |
| | *SmU*SmU | UU | OSS |
| WV-38123 | mUfUmAfUmAfGmAfGmCfAmAmGmAfAmCfAmC*SfUmGfUmU | UUAUAGAGCAAGAACACUGUU | OOOOOOOOOOOOOOOOSOO |
| | *SmU*SmU | UU | OSS |
| WV-38124 | mUfUmAfUmAfGmAfGmCfAmAmGmAfAmCfAmCfU*SmGfUmU | UUAUAGAGCAAGAACACUGUU | OOOOOOOOOOOOOOOOOSO |
| | *SmU*SmU | UU | OSS |
| WV-38125 | mUfUmAfUmAfGmAfGmCfAmAmGmAfAmCfAmCfUmG*SfUmU | UUAUAGAGCAAGAACACUGUU | OOOOOOOOOOOOOOOOOOS |
| | *SmU*SmU | UU | OSS |
| WV-38126 | mUfUmAfUmAfGmAfGmCfAmAmGmAfAmCfAmCfUmGfU*SmU | UUAUAGAGCAAGAACACUGUU | OOOOOOOOOOOOOOOOOOO |
| | *SmU*SmU | UU | SSS |
| WV-38127 | mU*fUmAfUmAfGmAfGmCfAmAmGmAfAmCfAmCfUmGfUmU* | UUAUAGAGCAAGAACACUGUU | XOOOOOOOOOOOOOOOOOO |
| | SmU*SmU | UU | OSS |
| WV-38128 | mUfU*mAfUmAfGmAfGmCfAmAmGmAfAmCfAmCfUmGfUmU* | UUAUAGAGCAAGAACACUGUU | OXOOOOOOOOOOOOOOOOO |
| | SmU*SmU | UU | OSS |
| WV-38129 | mUfUmA*fUmAfGmAfGmCfAmAmGmAfAmCfAmCfUmGfUmU* | UUAUAGAGCAAGAACACUGUU | OOXOOOOOOOOOOOOOOOO |
| | SmU*SmU | UU | OSS |
| WV-38130 | mUfUmAfU*mAfGmAfGmCfAmAmGmAfAmCfAmCfUmGfUmU* | UUAUAGAGCAAGAACACUGUU | OOOXOOOOOOOOOOOOOOO |
| | SmU*SmU | UU | OSS |
| WV-38131 | mUfUmAfUmA*fGmAfGmCfAmAmGmAfAmCfAmCfUmGfUmU* | UUAUAGAGCAAGAACACUGUU | OOOOXOOOOOOOOOOOOOO |
| | SmU*SmU | UU | OSS |
| WV-38132 | mUfUmAfUmAfG*mAfGmCfAmAmGmAfAmCfAmCfUmGfUmU* | UUAUAGAGCAAGAACACUGUU | OOOOOXOOOOOOOOOOOOO |
| | SmU*SmU | UU | OSS |
| WV-38133 | mUfUmAfUmAfGmA*fGmCfAmAmGmAfAmCfAmCfUmGfUmU* | UUAUAGAGCAAGAACACUGUU | OOOOOOXOOOOOOOOOOOO |
| | SmU*SmU | UU | OSS |
| WV-38134 | mUfUmAfUmAfGmAfG*mCfAmAmGmAfAmCfAmCfUmGfUmU* | UUAUAGAGCAAGAACACUGUU | OOOOOOOXOOOOOOOOOOO |
| | SmU*SmU | UU | OSS |
| WV-38135 | mUfUmAfUmAfGmAfGmC*fAmAmGmAfAmCfAmCfUmGfUmU* | UUAUAGAGCAAGAACACUGUU | OOOOOOOOXOOOOOOOOOO |
| | SmU*SmU | UU | OSS |
| WV-38136 | mUfUmAfUmAfGmAfGmCfA*mAmGmAfAmCfAmCfUmGfUmU* | UUAUAGAGCAAGAACACUGUU | OOOOOOOOOXOOOOOOOOO |
| | SmU*SmU | UU | OSS |
| WV-38137 | mUfUmAfUmAfGmAfGmCfAmA*mGmAfAmCfAmCfUmGfUmU* | UUAUAGAGCAAGAACACUGUU | OOOOOOOOOOXOOOOOOOO |
| | SmU*SmU | UU | OSS |
| WV-38138 | mUfUmAfUmAfGmAfGmCfAmAmG*mAfAmCfAmCfUmGfUmU* | UUAUAGAGCAAGAACACUGUU | OOOOOOOOOOOXOOOOOOO |
| | SmU*SmU | UU | OSS |
| WV-38139 | mUfUmAfUmAfGmAfGmCfAmAmGmA*fAmCfAmCfUmGfUmU* | UUAUAGAGCAAGAACACUGUU | OOOOOOOOOOOOXOOOOOO |
| | SmU*SmU | UU | OSS |
| WV-38140 | mUfUmAfUmAfGmAfGmCfAmAmGmAfA*mCfAmCfUmGfUmU* | UUAUAGAGCAAGAACACUGUU | OOOOOOOOOOOOOXOOOOO |
| | SmU*SmU | UU | OSS |
| WV-38141 | mUfUmAfUmAfGmAfGmCfAmAmGmAfAmC*fAmCfUmGfUmU* | UUAUAGAGCAAGAACACUGUU | OOOOOOOOOOOOOOXOOOO |
| | SmU*SmU | UU | OSS |
| WV-38142 | mUfUmAfUmAfGmAfGmCfAmAmGmAfAmCfA*mCfUmGfUmU* | UUAUAGAGCAAGAACACUGUU | OOOOOOOOOOOOOOOXOOO |
| | SmU*SmU | UU | OSS |
| WV-38143 | mUfUmAfUmAfGmAfGmCfAmAmGmAfAmCfAmC*fUmGfUmU* | UUAUAGAGCAAGAACACUGUU | OOOOOOOOOOOOOOOOXOO |
| | SmU*SmU | UU | OSS |
| WV-38144 | mUfUmAfUmAfGmAfGmCfAmAmGmAfAmCfAmCfU*mGfUmU* | UUAUAGAGCAAGAACACUGUU | OOOOOOOOOOOOOOOOOXO |
| | SmU*SmU | UU | OSS |
| WV-38145 | mUfUmAfUmAfGmAfGmCfAmAmGmAfAmCfAmCfUmG*fUmU* | UUAUAGAGCAAGAACACUGUU | OOOOOOOOOOOOOOOOOOX |
| | SmU*SmU | UU | OSS |
| WV-38146 | mUfUmAfUmAfGmAfGmCfAmAmGmAfAmCfAmCfUmGfU*mU* | UUAUAGAGCAAGAACACUGUU | OOOOOOOOOOOOOOOOOOO |
| | SmU*SmU | UU | XSS |
| WV-38678 | mU*fU*mAn001fUmAfGmAfGmCfAn001mAmGmAfAmCfAmC | UUAUAGAGCAAGAACACUGUU | XXnXOOOOOOnXOOOOOOO |
| | fUmGfUmU*mU*mU | UU | OOOXX |
| WV-38687 | mU*fU*mAn001fUmAfGmAfGmCfAmAmGmAfAmCfAmCfUmG | UUAUAGAGCAAGAACACUGUU | XXnXOOOOOOOOOOOOOOO |
| | n001fUmU*mU*mU | UU | nXOXX |
| WV-38703 | mU*RfU*SmAn001fUmAfGmAfGmCfAn001mAmGmAfAmCfA | UUAUAGAGCAAGAACACUGUU | RSnXOOOOOOnXOOOOOOO |
| | mCfUmGfUmU*SmU*SmU | UU | OOOSS |
| WV-38704 | mU*RfU*SmAn001fUmAfGmAfGmCfAn001mAmGmAfAmCfA | UUAUAGAGCAAGAACACUGUU | RSnXOOOOOOnXOOOOOOO |
| | mCfUmGn001fUmU*SmU*SmU | UU | OnXOSS |
| WV-38705 | mU*RfU*SmAn001RfUmAfGmAfGmCfAn001RmAmGmAfAmC | UUAUAGAGCAAGAACACUGUU | RSnROOOOOOnROOOOOOO |
| | fAmCfUmGfUmU*SmU*SmU | UU | OOOSS |
| WV-38706 | mU*RfU*SmAn001RfUmAfGmAfGmCfAn001RmAmGmAfAmC | UUAUAGAGCAAGAACACUGUU | RSnROOOOOOnROOOOOOO |
| | fAmCfUmGn001RfUmU*SmU*SmU | UU | OnROSS |
| WV-38707 | mU*RfU*SmAn001SfUmAfGmAfGmCfAn001SmAmGmAfAmC | UUAUAGAGCAAGAACACUGUU | RSnSOOOOOOnSOOOOOOO |
| | fAmCfUmGfUmU*SmU*SmU | UU | OOOSS |
| WV-40362 | Mod001L001mA*mAfCmAfGm UfGmUfUfCfUmUfGmCfUmC | AACAGUGUUCUUGCUCUAUAA | OXOOOOOOOOOOOOOOOOO |
| | fUmAfUmA*fA | | OX |
| WV-40363 | Mod001L001mA*SmAfCmAfGmUfGm UfUfCfUmUfGmCfUm | AACAGUGUUCUUGCUCUAUAA | osOOOOOOOOOOOOOOOOO |
| | CfUmAfUmA*SfA | | OS |
| WV-40552 | mU*RfU*SmAn001SfUmAfGmAfGmCfAn001RmAmGmAfAmC | UUAUAGAGCAAGAACACUGUU | RSnSOOOOOOnROOOOOOO |
| | fAmCfUmGn001RfUmU*SmU*SmU | UU | OnROSS |
| WV-40553 | mU*RfU*SmAn001RfUmAfGmAfGmCfAn001RmAmGmAfAmC | UUAUAGAGCAAGAACACUGUU | RSnROOOOOOnROOOOOOO |
| | fAmCfUmGn001SfUmU*SmU*SmU | UU | OnSOSS |
| WV-40555 | mU*RfU*SmAn001RfUmAfGmAfGmCfAn001SmAmGmAfAmC | UUAUAGAGCAAGAACACUGUU | RSnROOOOOOnSOOOOOOO |
| | fAmCfUmGn001SfUmU*SmU*SmU | UU | OnSOSS |
| WV-40556 | mU*RfU*SmAn001SfUmAfGmAfGmCfAn001RmAmGmAfAmC | UUAUAGAGCAAGAACACUGUU | RSnSOOOOOOnROOOOOOO |
| | fAmCfUmGn001SfUmU*SmU*SmU | UU | OnSOSS |
| WV-40796 | mU*RfU*SmAn001RfUmAfGmAfGmCfAn001SmAmGmAfAmC | UUAUAGAGCAAGAACACUGUU | RSnROOOOOOnSOOOOOOO |
| | fAmCfUmGn001RfUmU*SmU*SmU | UU | OnROSS |
| WV-40797 | mU*RfU*SmAn001SfUmAfGmAfGmCfAn001SmAmGmAfAmC | UUAUAGAGCAAGAACACUGUU | RSnSOOOOOOnSOOOOOOO |
| | fAmCfUmGn001RfUmU*SmU*SmU | UU | OnROSS |
| WV-40838 | mU*RfU*SmAn001SfUmAfGmAn001SfGmCfAn001SmAmGm | UUAUAGAGCAAGAACACUGUU | RSnSOOOnSOOnSOOOOOO |
| | AfAmCfAmCfUmGn001SfUmU*SmU*SmU | UU | OOnSOSS |
| WV-40839 | mU*RfU*SmAn001SfUmAfGmAfGn001SmCfAn001SmAmGm | UUAUAGAGCAAGAACACUGUU | RSnSOOOOnSOnSOOOOOO |
| | AfAmCfAmCfUmGn001SfUmU*SmU*SmU | UU | OOnSOSS |
| WV-40842 | mU*RfU*SmAn001SfUmAfGmAn001SfGmCfAn001SmAmGm | UUAUAGAGCAAGAACACUGUU | RSnSOOOnSOOnSOOOOOn |
| | AfAmCfAn001SmCfUmGn001SfUmU*SmU*SmU | UU | SOOnSOSS |
| WV-40843 | mU*RfU*SmAn001SfUmAfGmAfGn001SmCfAn001SmAmGm | UUAUAGAGCAAGAACACUGUU | RSnSOOOOnSOnSOOOOOn |
| | AfAmCfAn001SmCfUmGn001SfUmU*SmU*SmU | UU | SOOnSOSS |
| WV-41896 | mU*RfU*SmAn001RfUmAfGmAfGmCfAmAmGmAfAmCfAmCf | UUAUAGAGCAAGAACACUGUU | RSnROOOOOOOOOOOOOOO |
| | UmGfUmU*SmU*SmU | UU | OOSS |
| WV-41898 | mU*RfU*SmAfUmAn001RfGmAfGmCfAmAmGmAfAmCfAmCf | UUAUAGAGCAAGAACACUGUU | RSOOnROOOOOOOOOOOOO |
| | UmGfUmU*SmU*SmU | UU | OOSS |
| WV-41903 | mU*RfU*SmAfUmAfGmAfGmCfAn001RmAmGmAfAmCfAmCf | UUAUAGAGCAAGAACACUGUU | RSOOOOOOOnROOOOOOOO |
| | UmGfUmU*SmU*SmU | UU | OOSS |
| WV-41912 | mU*RfU*SmAfUmAfGmAfGmCfAmAmGmAfAmCfAmCfUmGn0 | UUAUAGAGCAAGAACACUGUU | RSOOOOOOOOOOOOOOOOn |
| | 01RfUmU*SmU*SmU | UU | ROSS |
| WV-41918 | mU*RfU*SmAn001SfUmAfGmAfGmCfAmAmGmAfAmCfAmCf | UUAUAGAGCAAGAACACUGUU | RSSOOOOOOOOOOOOOOO |
| | UmGfUmU*SmU*SmU | UU | OOSS |
| WV-41920 | mU*RfU*SmAfUmAn001SfGmAfGmCfAmAmGmAfAmCfAmCf | UUAUAGAGCAAGAACACUGUU | RSOOnSOOOOOOOOOOOOO |
| | UmGfUmU*SmU*SmU | UU | OOSS |
| WV-41925 | mU*RfU*SmAfUmAfGmAfGmCfAn001SmAmGmAfAmCfAmCf | UUAUAGAGCAAGAACACUGUU | RSOOOOOOOnSOOOOOOOO |
| | UmGfUmU*SmU*SmU | UU | OOSS |
| WV-41934 | mU*RfU*SmAfUmAfGmAfGmCfAmAmGmAfAmCfAmCfUmGn0 | UUAUAGAGCAAGAACACUGUU | RSOOOOOOOOOOOOOOOOn |
| | 01SfUmU*SmU*SmU | UU | SOSS |
| WV-41940 | mU*SfU*RmAn001RfUmAfGmAfGmCfAmAmGmAfAmCfAmCf | UUAUAGAGCAAGAACACUGUU | SRnROOOOOOOOOOOOOOO |
| | UmGfUmU*SmU*SmU | UU | OOSS |
| WV-41942 | mU*SfU*RmAfUmAn001RfGmAfGmCfAmAmGmAfAmCfAmCf | UUAUAGAGCAAGAACACUGUU | SROOnROOOOOOOOOOOOO |
| | UmGfUmU*SmU*SmU | UU | OOSS |
| WV-41947 | mU*SfU*RmAfUmAfGmAfGmCfAn001RmAmGmAfAmCfAmCf | UUAUAGAGCAAGAACACUGUU | SROOOOOOOnROOOOOOOO |
| | UmGfUmU*SmU*SmU | UU | OOSS |
| WV-41956 | mU*SfU*RmAfUmAfGmAfGmCfAmAmGmAfAmCfAmCfUmGn0 | UUAUAGAGCAAGAACACUGUU | SROOOOOOOOOOOOOOOOn |
| | 01RfUmU*SmU*SmU | UU | ROSS |
| WV-41962 | mU*SfU*RmAn001SfUmAfGmAfGmCfAmAmGmAfAmCfAmCf | UUAUAGAGCAAGAACACUGUU | SRnSOOOOOOOOOOOOOOO |
| | UmGfUmU*SmU*SmU | UU | OOSS |
| WV-41964 | mU*SfU*RmAfUmAn001SfGmAfGmCfAmAmGmAfAmCfAmCf | UUAUAGAGCAAGAACACUGUU | SROOnSOOOOOOOOOOOOO |
| | UmGfUmU*SmU*SmU | UU | OOSS |
| WV-41969 | mU*SfU*RmAfUmAfGmAfGmCfAn001SmAmGmAfAmCfAmCf | UUAUAGAGCAAGAACACUGUU | SROOOOOOOnSOOOOOOOO |
| | UmGfUmU*SmU*SmU | UU | OOSS |
| WV-41978 | mU*SfU*RmAfUmAfGmAfGmCfAmAmGmAfAmCfAmCfUmGn0 | UUAUAGAGCAAGAACACUGUU | SROOOOOOOOOOOOOOOOn |
| | 01SfUmU*SmU*SmU | UU | SOSS |
| WV-43987 | mU*SfU*RfAn001SmUmAfGmAmGmCfAn001SmAmGmAfAmC | UUAUAGAGCAAGAACACUGUU | SRnSOOOOOOnSOOOOOOO |
| | fAmCmUfGn001SmUmU*SmU*SmU | UU | OnSOSS |
| WV-43990 | mU*SfU*RfAn001SfUmAfGmAmGmCfAn001SfAmGmAfAmC | UUAUAGAGCAAGAACACUGUU | SRnSOOOOOOnSOOOOOOO |
| | fAmCmUfGn001SfUmU*SmU*SmU | UU | OnSOSS |
| WV-43991 | mU*SfU*RmAn001SmUmAfGmAfGmCmAn001SmAmGmAfAmC | UUAUAGAGCAAGAACACUGUU | SRnSOOOOOOnSOOOOOOO |
| | fAmCfUmGn001SmUmU*SmU*SmU | UU | OnSOSS |
| WV-43992 | mU*SfU*RfAn001SmUmAfGmAfGmCfAn001SmAmGmAfAmC | UUAUAGAGCAAGAACACUGUU | SRnSOOOOOOnSOOOOOOO |
| | fAmCfUfGn001SmUmU*SmU*SmU | UU | OnSOSS |
| WV-43993 | mU*SfU*RmAn001SfUmAfGmAfGmCmAn001SfAmGmAfAmC | UUAUAGAGCAAGAACACUGUU | SRnSOOOOOOnSOOOOOOO |
| | fAmCfUmGn001SfUmU*SmU*SmU | UU | OnSOSS |
| WV-49611 | mU*fU*mAfUmAfGmAmGmCmAfAmGmAfAmCfAmCmUmGmUmU | UUAUAGAGCAAGAACACUGUU | XXOOOOOOOOOOOOOOOOO |
| | *mU*mU | UU | OXX |
| WV-49612 | mU*fU*mAn001fUmAfGmAmGmCmAn001fAmGmAfAmCfAmC | UUAUAGAGCAAGAACACUGUU | XXnXOOOOOOnXOOOOOOO |
| | mUmGmUmU*mU*mU | UU | OOOXX |
| WV-49613 | mU*SfC*RmCn001SfUmUfCmCmCmUmGn001SfAmAmGfGmU | UCCUUCCCUGAAGGUUCCUCC | SRnSOOOOOOnSOOOOOOO |
| | fUmCmCmUmCmC*SmU*SmU | UU | OOOSS |
| WV-49614 | mU*SfC*RmCn001SfUmUfCmCmCmUmGn001RfAmAmGfGmU | UCCUUCCCUGAAGGUUCCUCC | SRnSOOOOOOnROOOOOOO |
| | fUmCmCmUmCmC*SmU*SmU | UU | OOOSS |
| WV-49615 | Mod001L001mG*SmGmAmGmGmAfAmCfCfUfUmCmAmGmGmG | GGAGGAACCUUCAGGGAAGGA | OSOOOOOOOOOOOOOOOOO |
| | mAmAmGmG*SmA | | OS |
| WV-49626 | mU*SfU*RmAn001fUmAfGmAmGmCmAn001fAmGmAfAmCfA | UUAUAGAGCAAGAACACUGUU | SRnXOOOOOOnXOOOOOOO |
| | mCmUmGmUmU*SmU*SmU | UU | OOOSS |
| WV-49900 | mU*fC*mCfUmUfCmCfCmUfGmAmAmGfGmUfUmCfCmUfCmC | UCCUUCCCUGAAGGUUCCUCC | XXOOOOOOOOOOOOOOOOO |
| | *mU*mU | UU | OXX |
| WV-49901 | Mod001L001mG*mGfAmGfGmAfAmCfCfUfUmCfAmGfGmGf | GGAGGAACCUUCAGGGAAGGA | OXOOOOOOOOOOOOOOOOO |
| | AmAfGmG*fA | | OX |
| WV-50034 | mU*SfU*RmAn003SfUmAfGmAmGmCmAn003RfAmGmAfAmC | UUAUAGAGCAAGAACACUGUU | SRnSOOOOOOnROOOOOOO |
| | fAmCmUmGmUmU*SmU*SmU | UU | OOOSS |
| WV-50035 | mU*SfU*RmAn003SfUmAfGmAmGmCmAn003SfAmGmAfAmC | UUAUAGAGCAAGAACACUGUU | SRnSOOOOOOnSOOOOOOO |
| | fAmCmUmGmUmU*SmU*SmU | UU | OOOSS |
| WV-50036 | mU*SfU*RmAn004SfUmAfGmAmGmCmAn004RfAmGmAfAmC | UUAUAGAGCAAGAACACUGUU | SRnSOOOOOOnROOOOOOO |
| | fAmCmUmGmUmU*SmU*SmU | UU | OOOSS |
| WV-50037 | mU*SfU*RmAn004SfUmAfGmAmGmCmAn004SfAmGmAfAmC | UUAUAGAGCAAGAACACUGUU | SRnSOOOOOOnSOOOOOOO |
| | fAmCmUmGmUmU*SmU*SmU | UU | OOOSS |
| WV-50040 | mU*SfU*RmAn008SfUmAfGmAmGmCmAn008RfAmGmAfAmC | UUAUAGAGCAAGAACACUGUU | SRnSOOOOOOnROOOOOOO |
| | fAmCmUmGmUmU*SmU*SmU | UU | OOOSS |
| WV-50041 | mU*SfU*RmAn008SfUmAfGmAmGmCmAn008SfAmGmAfAmC | UUAUAGAGCAAGAACACUGUU | SRnSOOOOOOnSOOOOOOO |
| | fAmCmUmGmUmU*SmU*SmU | UU | OOOSS |
| WV-50042 | mU*SfU*RmAn025SfUmAfGmAmGmCmAn025RfAmGmAfAmC | UUAUAGAGCAAGAACACUGUU | SRnSOOOOOOnROOOOOOO |
| | fAmCmUmGmUmU*SmU*SmU | UU | OOOSS |
| WV-50043 | mU*SfU*RmAn025SfUmAfGmAmGmCmAn025SfAmGmAfAmC | UUAUAGAGCAAGAACACUGUU | SRnSOOOOOOnSOOOOOOO |
| | fAmCmUmGmUmU*SmU*SmU | UU | OOOSS |
| WV-50044 | mU*SfU*RmAn026SfUmAfGmAmGmCmAn026RfAmGmAfAmC | UUAUAGAGCAAGAACACUGUU | SRnSOOOOOOnROOOOOOO |
| | fAmCmUmGmUmU*SmU*SmU | UU | OOOSS |
| WV-50045 | mU*SfU*RmAn026SfUmAfGmAmGmCmAn026SfAmGmAfAmC | UUAUAGAGCAAGAACACUGUU | SRnSOOOOOOnSOOOOOOO |
| | fAmCmUmGmUmU*SmU*SmU | UU | OOOSS |
| WV-50046 | mU*SfU*RmAn043SfUmAfGmAmGmCmAn043RfAmGmAfAmC | UUAUAGAGCAAGAACACUGUU | SRnSOOOOOOnROOOOOOO |
| | fAmCmUmGmUmU*SmU*SmU | UU | OOOSS |
| WV-50047 | mU*SfU*RmAn043SfUmAfGmAmGmCmAn043SfAmGmAfAmC | UUAUAGAGCAAGAACACUGUU | SRnSOOOOOOnSOOOOOOO |
| | fAmCmUmGmUmU*SmU*SmU | UU | OOOSS |
| WV-50048 | mU*SfU*RmAn058SfUmAfGmAmGmCmAn058RfAmGmAfAmC | UUAUAGAGCAAGAACACUGUU | SRnSOOOOOOnROOOOOOO |
| | fAmCmUmGmUmU*SmU*SmU | UU | OOOSS |
| WV-50049 | mU*SfU*RmAn058SfUmAfGmAmGmCmAn058SfAmGmAfAmC | UUAUAGAGCAAGAACACUGUU | SRnSOOOOOOnSOOOOOOO |
| | fAmCmUmGmUmU*SmU*SmU | UU | OOOSS |
| WV-50101 | 5mrpmU*RfU*SmAmUmAfGmAmGmCmAmAmGmAfAmCfAmCmU | UUAUAGAGCAAGAACACUGUU | RSOOOOOOOOOOOOOOOOO |
| | mGmUmU*SmU*SmU | UU | OSS |
| WV-50102 | 5mrpmU*SfU*RmAmUmAfGmAmGmCmAmAmGmAfAmCfAmCmU | UUAUAGAGCAAGAACACUGUU | SROOOOOOOOOOOOOOOOO |
| | mGmUmU*SmU*SmU | UU | OSS |
| WV-50103 | 5mrpmU*RfU*SmAn001SfUmAfGmAmGmCmAn001SfAmGmA | UUAUAGAGCAAGAACACUGUU | RSnSOOOOOOn$OOOOOOO |
| | fAmCfAmCmUmGmUmU*SmU*SmU | UU | OOOSS |
| WV-50104 | 5mrpmU*SfU*RmAn001SfUmAfGmAmGmCmAn001SfAmGmA | UUAUAGAGCAAGAACACUGUU | SRnSOOOOOOnSOOOOOOO |
| | fAmCfAmCmUmGmUmU*SmU*SmU | UU | OOOSS |
| WV-50105 | 5mrpmU*RfU*SmAn001SfUmAfGmAmGmCmAn001RfAmGmA | UUAUAGAGCAAGAACACUGUU | RSnSOOOOOOnROOOOOOO |
| | fAmCfAmCmUmGmUmU*SmU*SmU | UU | OOOSS |
| WV-50106 | 5mrpmU*SfU*RmAn001SfUmAfGmAmGmCmAn001RfAmGmA | UUAUAGAGCAAGAACACUGUU | SRnSOOOOOOnROOOOOOO |
| | fAmCfAmCmUmGmUmU*SmU*SmU | UU | OOOSS |
| WV-50108 | 5mvpmU*SfU*RmAmUmAfGmAmGmCmAmAmGmAfAmCfAmCmU | UUAUAGAGCAAGAACACUGUU | SROOOOOOOOOOOOOOOOO |
| | mGmUmU*SmU*SmU | UU | OSS |
| WV-50110 | 5mvpmU*SfU*RmAn001SfUmAfGmAmGmCmAn001SfAmGmA | UUAUAGAGCAAGAACACUGUU | SRnSOOOOOOnSOOOOOOO |
| | fAmCfAmCmUmGmUmU*SmU*SmU | UU | OOOSS |
| WV-50112 | 5mvpmU*SfU*RmAn001SfUmAfGmAmGmCmAn001RfAmGmA | UUAUAGAGCAAGAACACUGUU | SRnSOOOOOOnROOOOOOO |
| | fAmCfAmCmUmGmUmU*SmU*SmU | UU | OOOSS |
| WV-50113 | mU*SfU*RmAn001SfUmAfGmAmGmCmAn009RfAmGmAfAmC | UUAUAGAGCAAGAACACUGUU | SRnSOOOOOOnROOOOOOO |
| | fAmCmUmGmUmU*SmU*SmU | UU | OOOSS |
| WV-50114 | mU*SfU*RmAn001SfUmAfGmAmGmCmAn009SfAmGmAfAmC | UUAUAGAGCAAGAACACUGUU | SRnSOOOOOOnSOOOOOOO |
| | fAmCmUmGmUmU*SmU*SmU | UU | OOOSS |
| WV-50115 | mU*SfU*RmAn001SfUmAfGmAmGmCmAn033RfAmGmAfAmC | UUAUAGAGCAAGAACACUGUU | SRnSOOOOOOnROOOOOOO |
| | fAmCmUmGmUmU*SmU*SmU | UU | OOOSS |
| WV-50116 | mU*SfU*RmAn001SfUmAfGmAmGmCmAn033SfAmGmAfAmC | UUAUAGAGCAAGAACACUGUU | SRnSOOOOOOnSOOOOOOO |
| | fAmCmUmGmUmU*SmU*SmU | UU | OOOSS |
| WV-50481 | mU*fU*mAn001fUmAfGmAmGmCmAn001fAmGmAfAmCfAmC | UUAUAGAGCAAGAACACUGUU | XXnXOOOOOOnXOOOOOOO |
| | mUmGn001fUmU*mU*mU | UU | OnXOXX |
| WV-50482 | mU*fU*mAn001fUmAfGmAmGmCfAn001mAmGmAfAmCfAmC | UUAUAGAGCAAGAACACUGUU | XXnXOOOOOOnXOOOOOOO |
| | mUmGn001fUmU*mU*mU | UU | OnXOXX |
| WV-50485 | mU*SfU*RmAn001fUmAfGmAmGmCmAn001fAmGmAfAmCfA | UUAUAGAGCAAGAACACUGUU | SRnXOOOOOOnXOOOOOOO |
| | mCmUmGn001fUmU*SmU*SmU | UU | OnXOSS |
| WV-50486 | mU*SfU*RmAn001fUmAfGmAmGmCfAn001mAmGmAfAmCfA | UUAUAGAGCAAGAACACUGUU | SRnXOOOOOOnXOOOOOOO |
| | mCmUmGn001fUmU*SmU*SmU | UU | OnXOSS |
| WV-51122 | mU*SfU*RmAfUmAfGmAmGmCmAfAmGmAfAmCfAmCmUmGmU | UUAUAGAGCAAGAACACUGUU | SROOOOOOOOOOOOOOOOO |
| | mU*SmU*SmU | UU | OSS |

TABLE 1a
Example Oligonucleotides/Compositions for non-targeting controls.

| | | | Stereochemistry/ |
|---|---|---|---|
| ID | Description | Naked Sequence | linkage |
| WV-49613 | mU*SfC*RmCn001SfUmUfCmCmCmUmGn001SfAmAmGfGmU | UCCUUCCCUGAAGG | SRnSOOOOOOnSOOOOOOOO |
| | fUmCmCmUmCmC*SmU*SmU | UUCCUCCUU | OOSS |
| WV-49614 | mU*SfC*RmCn001SfUmUfCmCmCmUmGn001RfAmAmGfGmU | UCCUUCCCUGAAGG | SRnSOOOOOOnROOOOOOO |
| | fUmCmCmUmCmC*SmU*SmU | UUCCUCCUU | OOOSS |
| WV-49615 | Mod001L001mG*SmGmAmGmGmAfAmCfCfUfUmCmAmGmG | GGAGGAACCUUCAG | OSOOOOOOOOOOOOOOOO |
| | mGmAmAmGmG*SmA | GGAAGGA | OOS |
| WV-49900 | mU*fC*mCfUmUfCmCfCmUfGmAmAmGfGmUfUmCfCmUfCm | UCCUUCCCUGAAGG | XXOOOOOOOOOOOOOOOO |
| | C*mU*mU | UUCCUCCUU | OOXX |
| WV-49901 | Mod001L001mG*mGfAmGfGmAfAmCfCfUfUmCfAmGfGmGfA | GGAGGAACCUUCAG | OXOOOOOOOOOOOOOOOO |
| | mAfGmG*fA | GGAAGGA | OOX |
| WV-49903 | mU*fC*mCmUmUfCmCmCmUmGmAmAmGfGmUfUmCmCmU | UCCUUCCCUGAAGG | XXOOOOOOOOOOOOOOOO |
| | mCmC*mU*mU | UUCCUCCUU | OOXX |
| WV-49904 | Mod001L001mG*mGmAmGmGmAfAmCfCfUfUmCmAmGmG | GGAGGAACCUUCAG | OXOOOOOOOOOOOOOOOO |
| | mGmAmAmGmG*mA | GGAAGGA | OOX |

TABLE 1b
Example Oligonucleotides/Compositions that target TTR.

| | | Naked |
|---|---|---|
| ID | Description | Sequence |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106266 | (A)p.m(G)p.m(C)p.m(A)[n001S].[fl2r](A)p.m(G)[n001S].m(A)[n001S].[fl2r] | AGAACACUGU |
| | (A)p.m(C)p.[fl2r](A)p.m(C)p.m(U)p.m(G)p.m(U)p.m(U)[Ssp].m(U)[Ssp].m(U)} | UUU |
| | $$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106267 | (A)p.m(G)p.m(C)p.m(A)[n001S].[fl2r](A)p.m(G)p.m(A)[n001S].[fl2r](A) | AGAACACUGU |
| | [n001S].m(C)p.[fl2r](A)p.m(C)p.m(U)p.m(G)p.m(U)p.m(U)[Ssp].m(U)[Ssp].m | UUU |
| | (U)}$$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106268 | (A)p.m(G)p.m(C)p.m(A)[n001S].[fl2r](A)p.m(G)p.m(A)p.[fl2r](A)[n001S]. | AGAACACUGU |
| | m(C)[n001S].[fl2r](A)p.m(C)p.m(U)p.m(G)p.m(U)p.m(U)[Ssp].m(U)[Ssp].m | UUU |
| | (U)}$$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106269 | (A)p.m(G)p.m(C)p.m(A)[n001S].[fl2r](A)p.m(G)p.m(A)p.[fl2r](A)p.m(C) | AGAACACUGU |
| | [n001S].[fl2r](A)[n001S].m(C)p.m(U)p.m(G)p.m(U)p.m(U)[Ssp].m(U)[Ssp].m | UUU |
| | (U)}$$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106270 | (A)p.m(G)p.m(C)p.m(A)[n001S].[fl2r](A)p.m(G)p.m(A)p.[fl2r](A)p.m(C)p. | AGAACACUGU |
| | [fl2r](A)[n001S].m(C)[n001S].m(U)p.m(G)p.m(U)p.m(U)[Ssp].m(U)[Ssp].m(U) | UUU |
| | }$$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106271 | (A)p.m(G)p.m(C)p.m(A)[n001S].[fl2r](A)p.m(G)p.m(A)p.[fl2r](A)p.m(C)p. | AGAACACUGU |
| | [fl2r](A)p.m(C)[n001S].m(U)[n001S].m(G)p.m(U)p.m(U)[Ssp].m(U)[Ssp]. | UUU |
| | m(U)}$$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106272 | (A)p.m(G)p.m(C)p.m(A)[n001S].[fl2r](A)p.m(G)p.m(A)p.[fl2r](A)p.m(C)p. | AGAACACUGU |
| | [fl2r](A)p.m(C)p.m(U)[n001S].m(G)[n001S].m(U)p.m(U)[Ssp].m(U)[Ssp]. | UUU |
| | m(U)}$$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106273 | (A)p.m(G)p.m(C)p.m(A)[n001S].[fl2r](A)p.m(G)p.m(A)p.[fl2r](A)p.m(C)p. | AGAACACUGU |
| | [fl2r](A)p.m(C)p.m(U)p.m(G)[n001S].m(U)[n001S].m(U)[Ssp].m(U)[Ssp]. | UUU |
| | m(U)}$$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106274 | (A)p.m(G)p.m(C)p.m(A)[n001S].[fl2r](A)p.m(G)[n001S].m(A)[n001S].[fl2r] | AGAACACUGU |
| | (A)[n001S].m(C)p.[fl2r](A)p.m(C)p.m(U)p.m(G)p.m(U)p.m(U)[Ssp].m(U) | UUU |
| | [Ssp].m(U)}$$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106275 | (A)p.m(G)p.m(C)p.m(A)[n001S].[fl2r](A)p.m(G)p.m(A)[n001S].[fl2r](A) | AGAACACUGU |
| | [n001S].m(C)[n001S].[fl2r](A)p.m(C)p.m(U)p.m(G)p.m(U)p.m(U)[Ssp].m(U) | UUU |
| | [Ssp].m(U)}$$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106276 | (A)p.m(G)p.m(C)p.m(A)[n001S].[fl2r](A)p.m(G)p.m(A)p.[fl2r](A)[n001S].m | AGAACACUGU |
| | (C)[n001S].[fl2r](A)[n001S].m(C)p.m(U)p.m(G)p.m(U)p.m(U)[Ssp].m(U)[Ssp]. | UUU |
| | m(U)}$$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106277 | (A)p.m(G)p.m(C)p.m(A)[n001S].[fl2r](A)p.m(G)p.m(A)p.[fl2r](A)p.m(C) | AGAACACUGU |
| | [n001S].[fl2r](A)[n001S].m(C)[n001S].m(U)p.m(G)p.m(U)p.m(U)[Ssp].m(U) | UUU |
| | [Ssp].m(U)}$$$$V2.0 | |
| SSR- | RNA1{m(U)[Ssp].[fl2r](U)[Rsp].m(A)[n001S].[fl2r](U)p.m(A)p.[fl2r](G)p. | UUAUAGAGCA |
| 0106278 | m(A)p.m(G)p.m(C)p.m(A)[n001S].[fl2r](A)p.m(G)p.m(A)p.[fl2r](A)p.m(C)p. | AGAACACUGU |
| | [fl2r](A)[n001S].m(C)[n001S].m(U)[n001S].m(G)p.m(U)p.m(U)[Ssp].m(U)[Ssp]. | UUU |
| | m(U)}$$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106279 | (A)p.m(G)p.m(C)p.m(A)[n001S].[fl2r](A)p.m(G)p.m(A)p.[fl2r](A)p.m(C)p. | AGAACACUGU |
| | [fl2r](A)p.m(C)[n001S].m(U)[n001S].m(G)[n001S].m(U)p.m(U)[Ssp].m(U) | UUU |
| | [Ssp].m(U)}$$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106280 | (A)p.m(G)p.m(C)p.m(A)[n001S].[fl2r](A)p.m(G)p.m(A)p.[fl2r](A)p.m(C)p. | AGAACACUGU |
| | [fl2r](A)p.m(C)p.m(U)[n001S].m(G)[n001S].m(U)[n001S].m(U)[Ssp].m(U) | UUU |
| | [Ssp].m(U)}$$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106281 | (A)p.m(G)p.m(C)p.m(A)[n001S].[fl2r](A)p.m(G)[n001S].m(A)[n001S].[fl2r] | AGAACACUGU |
| | (A)[n001S].m(C)[n001S].[fl2r](A)p.m(C)p.m(U)p.m(G)p.m(U)p.m(U)[Ssp]. | UUU |
| | m(U)[Ssp].m(U)}$$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106282 | (A)p.m(G)p.m(C)p.m(A)[n001S].[fl2r](A)p.m(G)p.m(A)[n001S].[fl2r](A) | AGAACACUGU |
| | [n001S].m(C)[n001S].[fl2r](A)[n001S].m(C)p.m(U)p.m(G)p.m(U)p.m(U)[Ssp]. | UUU |
| | m(U)[Ssp].m(U)}$$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106283 | (A)p.m(G)p.m(C)p.m(A)[n001S].[fl2r](A)p.m(G)p.m(A)p.[fl2r](A)[n001S]. | AGAACACUGU |
| | m(C)[n001S].[fl2r](A)[n001S].m(C)[n001S].m(U)p.m(G)p.m(U)p.m(U)[Ssp]. | UUU |
| | m(U)[Ssp].m(U)}$$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106284 | (A)p.m(G)p.m(C)p.m(A)[n001S].[fl2r](A)p.m(G)p.m(A)p.[fl2r](A)p.m(C) | AGAACACUGU |
| | [n001S].[fl2r](A)[n001S].m(C)[n001S].m(U)[n001S].m(G)p.m(U)p.m(U)[Ssp]. | UUU |
| | m(U)[Ssp].m(U)}$$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106285 | (A)p.m(G)p.m(C)p.m(A)[n001S].[fl2r](A)p.m(G)p.m(A)p.[fl2r](A)p.m(C)p. | AGAACACUGU |
| | [fl2r](A)[n001S].m(C)[n001S].m(U)[n001S].m(G)[n001S].m(U)p.m(U)[Ssp]. | UUU |
| | m(U)[Ssp].m(U)}$$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106286 | (A)p.m(G)p.m(C)p.m(A)[n001S].[fl2r](A)p.m(G)p.m(A)p.[fl2r](A)p.m(C)p. | AGAACACUGU |
| | [fl2r](A)p.m(C)[n001S].m(U)[n001S].m(G)[n001S].m(U)[n001S].m(U)[Ssp]. | UUU |
| | m(U)[Ssp].m(U)}$$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106287 | (A)p.m(G)p.m(C)p.m(A)[n001S].[fl2r](A)p.m(G)[n001S].m(A)p.[fl2r](A) | AGAACACUGU |
| | [n001S].m(C)p.[fl2r](A)[n001S].m(C)p.m(U)[n001S].m(G)p.m(U)[n001S].m(U) | UUU |
| | [Ssp].m(U)[Ssp].m(U)}$$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106288 | (A)p.m(G)p.m(C)p.m(A)[n001S].[fl2r](A)p.m(G)p.m(A)[n001S].[fl2r](A)p.m | AGAACACUGU |
| | (C)[n001S].[fl2r](A)p.m(C)[n001S].m(U)p.m(G)[n001S].m(U)p.m(U)[Ssp].m(U) | UUU |
| | [Ssp].m(U)}$$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106289 | (A)p.m(G)p.m(C)p.m(A)[n001S].[fl2r](A)p.m(G)[n001S].[fl2r](A)p.[fl2r] | AGAACACUGU |
| | (A)[n001S].[fl2r](C)p.[fl2r](A)[n001S].[fl2r](C)p.m(U)[n001S].[fl2r](G)p. | UUU |
| | m(U)[n001S].[fl2r](U)[Ssp].m(U)[Ssp].m(U)}$$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106290 | (A)p.m(G)p.m(C)p.m(A)[n001S].[fl2r](A)p.m(G)p.m(A)[n001S].[fl2r](A)p. | AGAACACUGU |
| | m(C)[n001S].[fl2r](A)p.m(C)[n001S].[fl2r](U)p.m(G)[n001S].[fl2r](U)p.m(U) | UUU |
| | [Ssp].m(U)[Ssp].m(U)}$$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106291 | (A)p.m(G)p.m(C)p.m(A)[n001S].[fl2r](A)p.m(G)p.m(A)[n001S].[fl2r](A) | AGAACACUGU |
| | [n001S].m(C)p.[fl2r](A)p.m(C)[n001S].m(U)[n001S].m(G)p.m(U)p.m(U)[Ssp].m | UUU |
| | (U)[Ssp].m(U)}$$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106292 | (A)p.m(G)p.m(C)p.m(A)[n001S].[fl2r](A)p.m(G)p.m(A)p.[fl2r](A)[n001S]. | AGAACACUGU |
| | m(C)[n001S].[fl2r](A)p.m(C)p.m(U)[n001S].m(G)[n001S].m(U)p.m(U)[Ssp].m | UUU |
| | (U)[Ssp].m(U)}$$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106293 | (A)p.m(G)p.m(C)p.m(A)[n001S].[fl2r](A)p.m(G)[n001S].m(A)p.[fl2r](A)p. | AGAACACUGU |
| | m(C)p.[fl2r](A)p.m(C)p.m(U)p.m(G)p.m(U)p.m(U)[Ssp].m(U)[Ssp].m(U)} | UUU |
| | $$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106294 | (A)p.m(G)p.m(C)p.m(A)[n001S].[fl2r](A)p.m(G)p.m(A)[n001S].[fl2r](A)p.m | AGAACACUGU |
| | (C)p.[fl2r](A)p.m(C)p.m(U)p.m(G)p.m(U)p.m(U)[Ssp].m(U)[Ssp].m(U)} | UUU |
| | $$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106295 | (A)p.m(G)p.m(C)p.m(A)[n001S].[fl2r](A)p.m(G)p.m(A)p.[fl2r](A)[n001S]. | AGAACACUGU |
| | m(C)p.[fl2r](A)p.m(C)p.m(U)p.m(G)p.m(U)p.m(U)[Ssp].m(U)[Ssp].m(U)} | UUU |
| | $$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106296 | (A)p.m(G)p.m(C)p.m(A)[n001S].[fl2r](A)p.m(G)p.m(A)p.[fl2r](A)p.m(C) | AGAACACUGU |
| | [n001S].[fl2r](A)p.m(C)p.m(U)p.m(G)p.m(U)p.m(U)[Ssp].m(U)[Ssp].m(U)} | UUU |
| | $$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106297 | (A)p.m(G)p.m(C)p.m(A)[n001S].[fl2r](A)p.m(G)p.m(A)p.[fl2r](A)p.m(C)p. | AGAACACUGU |
| | [fl2r](A)[n001S].m(C)p.m(U)p.m(G)p.m(U)p.m(U)[Ssp].m(U)[Ssp].m(U)} | UUU |
| | $$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106298 | (A)p.m(G)p.m(C)p.m(A)[n001S].[fl2r](A)p.m(G)p.m(A)p.[fl2r](A)p.m(C)p. | AGAACACUGU |
| | [fl2r](A)p.m(C)[n001S].m(U)p.m(G)p.m(U)p.m(U)[Ssp].m(U)[Ssp].m(U)} | UUU |
| | $$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106299 | (A)p.m(G)p.m(C)p.m(A)[n001S].[fl2r](A)p.m(G)p.m(A)p.[fl2r](A)p.m(C)p. | AGAACACUGU |
| | [fl2r](A)p.m(C)p.m(U)[n001S].m(G)p.m(U)p.m(U)[Ssp].m(U)[Ssp].m(U)} | UUU |
| | $$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106300 | (A)p.m(G)p.m(C)p.m(A)[n001S].[fl2r](A)p.m(G)p.m(A)p.[fl2r](A)p.m(C)p. | AGAACACUGU |
| | [fl2r](A)p.m(C)p.m(U)p.m(G)[n001S].m(U)p.m(U)[Ssp].m(U)[Ssp].m(U)} | UUU |
| | $$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106301 | (A)p.m(G)p.m(C)p.m(A)[n001S].[fl2r](A)p.m(G)p.m(A)p.[fl2r](A)p.m(C)p. | AGAACACUGU |
| | [fl2r](A)p.m(C)p.m(U)p.m(G)p.m(U)[n001S].m(U)[Ssp].m(U)[Ssp].m(U)} | UUU |
| | $$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106302 | (A)p.m(G)p.m(C)p.m(A)[n001S].[fl2r](A)p.m(G)[n001S].[fl2r](A)p.[fl2r](A) | AGAACACUGU |
| | p.m(C)p.[fl2r](A)p.m(C)p.m(U)p.m(G)p.m(U)p.m(U)[Ssp].m(U)[Ssp].m(U)} | UUU |
| | $$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106303 | (A)p.m(G)p.m(C)p.m(A)[n001S].[fl2r](A)p.m(G)p.m(A)p.[fl2r](A)[n001S]. | AGAACACUGU |
| | [fl2r](C)p.[fl2r](A)p.m(C)p.m(U)p.m(G)p.m(U)p.m(U)[Ssp].m(U)[Ssp].m(U)} | UUU |
| | $$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106304 | (A)p.m(G)p.m(C)p.m(A)[n001S].[fl2r](A)p.m(G)p.m(A)p.[fl2r](A)p.m(C)p. | AGAACACUGU |
| | [fl2r](A)[n001S].[fl2r](C)p.m(U)p.m(G)p.m(U)p.m(U)[Ssp].m(U)[Ssp].m(U)} | UUU |
| | $$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106305 | (A)p.m(G)p.m(C)p.m(A)[n001S].[fl2r](A)p.m(G)p.m(A)p.[fl2r](A)p.m(C)p. | AGAACACUGU |
| | [fl2r](A)p.m(C)[n001S].[fl2r](U)p.m(G)p.m(U)p.m(U)[Ssp].m(U)[Ssp].m(U)} | UUU |
| | $$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106306 | (A)p.m(G)p.m(C)p.m(A)[n001S].[fl2r](A)p.m(G)p.m(A)p.[fl2r](A)p.m(C)p. | AGAACACUGU |
| | [fl2r](A)p.m(C)p.m(U)[n001S].[fl2r](G)p.m(U)p.m(U)[Ssp].m(U)[Ssp].m(U)} | UUU |
| | $$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0104474 | (A)p.m(G)p.m(C)p.m(A)[n001S].[fl2r](A)p.m(G)p.m(A)p.[fl2r](A)p.m(C)p. | AGAACACUGU |
| | [fl2r](A)p.m(C)p.m(U)p.m(G)[n001S].[fl2r](U)p.m(U)[Ssp].m(U)[Ssp].m(U)} | UUU |
| | $$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0106307 | (A)p.m(G)p.m(C)p.m(A)[n001S].[fl2r](A)p.m(G)p.m(A)p.[fl2r](A)p.m(C)p. | AGAACACUGU |
| | [fl2r](A)p.m(C)p.m(U)p.m(G)p.m(U)[n001S].[fl2r](U)[Ssp].m(U)[Ssp].m(U)} | UUU |
| | $$$$V2.0 | |
| SSR- | RNA1{m(U)[Ssp].[fl2r](U)[Rsp].m(A)[n001S].[fl2r](U)p.m(A)p.[fl2r](G)p.m | UUAUAGAGCA |
| 0104475 | (A)p.m(G)p.m(C)p.m(A)[n001S].[fl2r](A)p.m(G)p.m(A)p.[fl2r](A)p.m(C)p. | AGAACACUGU |
| | [fl2r](A)p.m(C)p.m(U)p.m(G)p.m(U)p.m(U)[Ssp].m(U)[Ssp].m(U)}$$$$V2.0 | UUU |
| SSR- | RNA1{m(U)[sp].[fl2r](U)[sp].m(A)p.m(U)p.m(A)p.[fl2r](G)p.m(A)p.m(G)p.m | UUAUAGAGCA |
| 0104720 | (C)p.m(A)p.m(A)p.m(G)p.m(A)p.[fl2r](A)p.m(C)p.[fl2r](A)p.m(C)p. | AGAACACUGU |
| | m(U)p.m(G)p.m(U)p.m(U)[sp].m(U)[sp].m(U)}$$$$V2.0 | UUU |
| SSR- | RNA1{p.m(A)[Ssp].m(A)p.m(C)p.m(A)p.m(G)p.m(U)p.[fl2r](G)p.m(U)p.[fl2r] | AACAGUGUUC |
| 0101599 | (U)p.[fl2r](C)p.[fl2r](U)p.m(U)p.m(G)p.m(C)p.m(U)p.m(C)p.m(U)p.m(A)p. | UUGCUCUAUA |
| | m(U)p.m(A)[Ssp].m(A)}\|CHEM1{[GalNAc3C12oyl]}\|CHEM2{[nC60]}$CHEM2, | A |
| | RNA1, 1: R1-1: R1\|CHEM2, CHEM1, 1: R2-1: R1$$$V2. | |
| SSR- | RNA1{p.m(A)[sp].m(A)p.m(C)p.m(A)p.m(G)p.m(U)p.[fl2r](G)p.m(U)p.[fl2r] | AACAGUGUUC |
| 0101596 | (U)p.[fl2r](C)p.[fl2r](U)p.m(U)p.m(G)p.m(C)p.m(U)p.m(C)p.m(U)p.m(A) | UUGCUCUAUA |
| | p.m(U)p.m(A)[sp].m(A)}\|CHEM1{[GalNAc3C12oyl]}\|CHEM2{[nC60]}$CHEM2, | A |
| | RNA1, 1: R1-1: R1\|CHEM2, CHEM1, 1: R2-1: R1$$$V2.0 | |

Notes:
SSR-0104474 = WV-43988
SSR-0104475 = WV-47145
SSR-0104720 = WV-41826
SSR-0101599 = WV-42080
SSR-0101596 = WV-41828

### Notes:

Description, Base Sequence and Stereochemistry/Linkage, due to their length, may be divided into multiple lines in Table 1. Unless otherwise specified, all oligonucleotides in Table 1 are single-stranded. As appreciated by those skilled in the art, nucleoside units are unmodified and contain unmodified nucleobases and 2′-deoxy sugars unless otherwise indicated (e.g., with r, m, etc.); linkages, unless otherwise indicated, are natural phosphate linkages; and acidic/basic groups may independently exist in their salt forms. If a sugar is not specified, the sugar is a natural DNA sugar; and if an internucleotidic linkage is not specified, the internucleotidic linkage is a natural phosphate linkage. Moieties and modifications:
m: 2′-OMe;
f or [fl2r]: 2′-F;
O, PO, p: phosphodiester (phosphate). It can a linkage or be an end group (or a component thereof), e.g., a linkage between a linker and an oligonucleotide chain, an internucleotidic linkage (a natural phosphate linkage), etc. Phosphodiesters are typically indicated with “O” in the Stereochemistry/Linkage column and are typically not marked in the Description column (if it is an end group, e.g., a 5′-end group, it is indicated in the Description and typically not in Stereochemistry/Linkage); if no linkage is indicated in the Description column, it is typically a phosphodiester unless otherwise indicated. Note that a phosphate linkage between a linker (e.g., L001) and an oligonucleotide chain may not be marked in the Description column, but may be indicated with “O” in the Stereochemistry/Linkage column;
*, PS, sp: Phosphorothioate. It can be an end group (if it is an end group, e.g., a 5′-end group, it is indicated in the Description and typically not in Stereochemistry/Linkage), or a linkage, e.g., a linkage between linker (e.g., L001) and an oligonucleotide chain, an internucleotidic linkage (a phosphorothioate internucleotidic linkage), etc.;
R, Rp, or [Rsp]: Phosphorothioate in the Rp configuration. Note that * R in Description indicates a single phosphorothioate linkage in the Rp configuration;
S, Sp, or [Ssp]: Phosphorothioate in the Sp configuration. Note that * S in Description indicates a single phosphorothioate linkage in the Sp configuration;
X: stereorandom phosphorothioate;
CHEM1: ligand;
CHEM2: 5′-linker;
   n001:

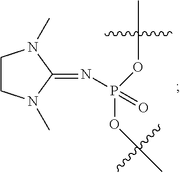 [SMILES: *OP(=O)(N=C1N(C)CCN1C)O*]

nX: stereorandom n001;
nR or n001R or [n001R]: n001 in Rp configuration;
nS or n001S or [n001S]: n001 in Sp configuration;
   n009:

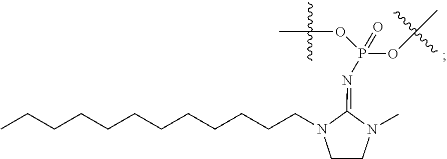 [SMILES: *OP(=O)(/N=C1\N(C)CCN1CCCCCCCCCCCC)O*]

nX: stereorandom n009;
nR or n009R: n009 in Rp configuration;
nS or n009S: n009 in Sp configuration;
   n031:

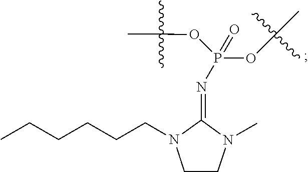 [SMILES: *OP(=O)(/N=C1\N(C)CCN1CCCCCC)O*]

nX: stereorandom n031;
nR or n031R: n031 in Rp configuration;
nS or n031S: n031 in Sp configuration;
   n033:

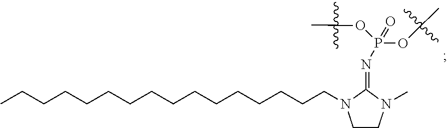 [SMILES: *OP(=O)(/N=C1\N(C)CCN1CCCCCCCCCCCCCCCC)O*]

nX: stereorandom n033;
nR or n033R: n033 in Rp configuration;
nS or n033S: n033 in Sp configuration;
   n037:

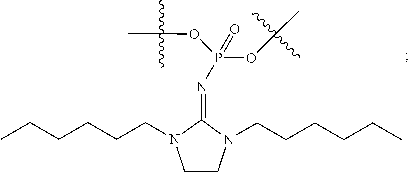 [SMILES: *OP(=O)(N=C1N(CCCCCC)CCN1CCCCCC)O*]

nX: stereorandom n037;
nR or n037R: n037 in Rp configuration;
nS or n037S: n037 in Sp configuration;
   n046:

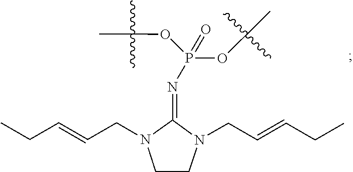 [SMILES: *OP(=O)(N=C1N(C/C=C/CC)CCN1C/C=C/CC)O*]

nX: stereorandom n046;
nR or n046R: n046 in Rp configuration;
nS or n046S: n046 in Sp configuration;
   n047:

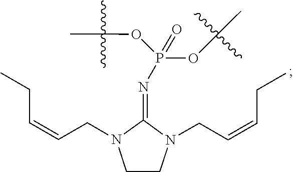 [SMILES: *OP(=O)(N=C1N(C/C=C\CC)CCN1C/C=C\CC)O*]

nX: stereorandom n047;
nR or n047R: n047 in Rp configuration;
nS or n047S: n047 in Sp configuration;
   n025:

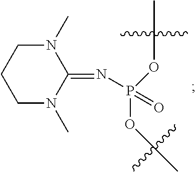 [SMILES: *OP(=O)(N=C1N(C)CCCN1C)O*]

nX: stereorandom n025;
nR or n025R: n025 in Rp configuration;
nS or n025S: n025 in Sp configuration;
   n054:

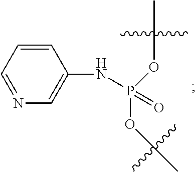 [SMILES: *OP(=O)(Nc1cccnc1)O*]

nX: stereorandom n054;
nR or n054R: n054 in Rp configuration;
nS or n054S: n054 in Sp configuration;
   n055:

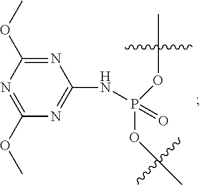 [SMILES: *OP(=O)(Nc1nc(OC)nc(OC)n1)O*]

nX: stereorandom n055;
nR or n055R: n055 in Rp configuration;
nS or n055S: n055 in Sp configuration;
   n026:

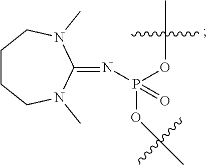 [SMILES: *OP(=O)(N=C1N(C)CCCCN1C)O*]

nX: stereorandom n001;
nR or n026R: n026 in Rp configuration;
nS or n026S: n026 in Sp configuration;
   n004:

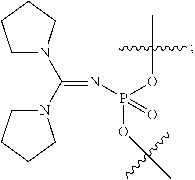 [SMILES: *OP(=O)(N=C(N1CCCC1)N1CCCC1)O*]

nX: stereorandom n004;
nR or n004R: n004 in Rp configuration;
nS or n004S: n004 in Sp configuration;
   n003:

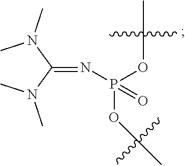 [SMILES: *OP(=O)(N=C(N(C)C)N(C)C)O*]

nX: stereorandom n003;
nR or n003R: n003 in Rp configuration;
nS or n003S: n003 in Sp configuration;
   n008:

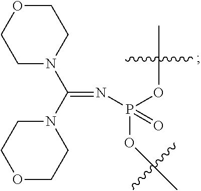 [SMILES: *OP(=O)(N=C(N1CCOCC1)N1CCOCC1)O*]

nX: stereorandom n008;
nR or n008R: n008 in Rp configuration;
nS or n008S: n008 in Sp configuration;
   n029:

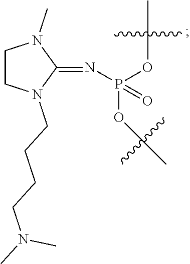 [SMILES: *OP(=O)(/N=C1/N(C)CCN1CCCCN(C)C)O*]

nX: stereorandom n029;
nR or n029R: n029 in Rp configuration;
nS or n029S: n029 in Sp configuration;
   n021:

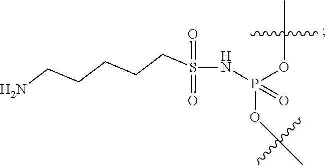 [SMILES: *OP(=O)(NS(=O)(=O)CCCCCN)O*]

nX: stereorandom n021;
nR or n021R: n021 in Rp configuration;
nS or n021S: n021 in Sp configuration;
   n006:

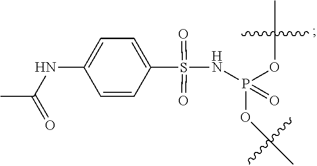 [SMILES: *OP(=O)(NS(=O)(=O)c1ccc(NC(C)=O)cc1)O*]

nX: stereorandom n006;
nR or n006R: n006 in Rp configuration;
nS or n006S: n006 in Sp configuration;
   n020:

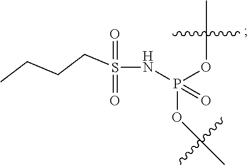 [SMILES: *OP(=O)(NS(=O)(=O)CCCC)O*]

nX: stereorandom n020;
nR or n020R: n020 in Rp configuration;
nS or n020S: n020 in Sp configuration;
   n043:

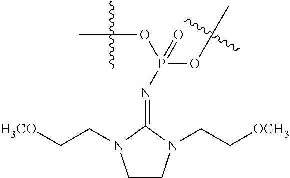 [SMILES: *OP(=O)(N=C1N(CCOC)CCN1CCOC)O*]

nX: stereorandom n043;
nR or n043R: n043 in Rp configuration;
nS or n043S: n043 in Sp configuration;
   n058:

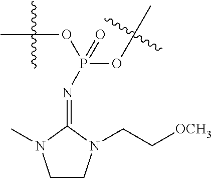 [SMILES: *OP(=O)(/N=C1/N(C)CCN1CCOC)O*]

nX: stereorandom n058;
nR or n058R: n058 in Rp configuration;
nS or n058S: n058 in Sp configuration;
X: stereorandom phosphorothioate;

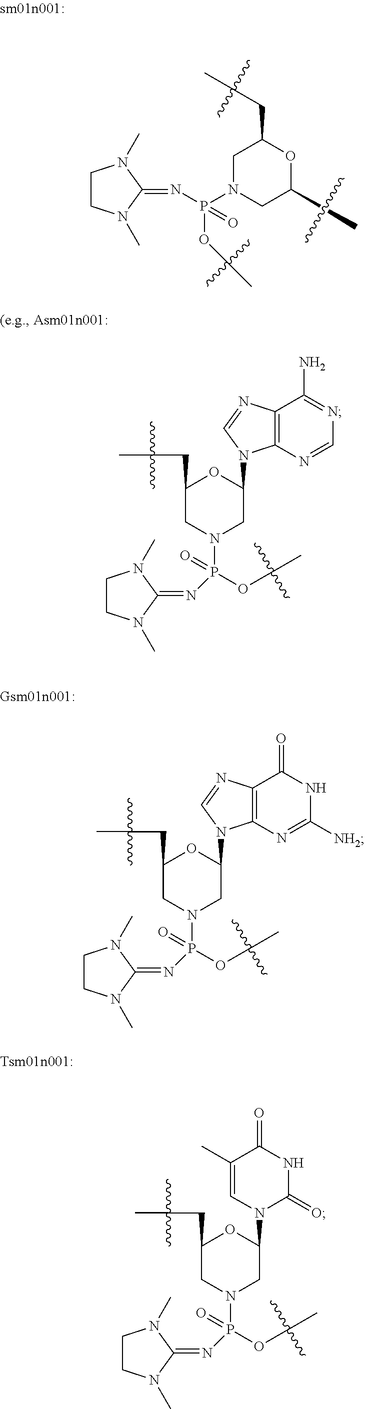

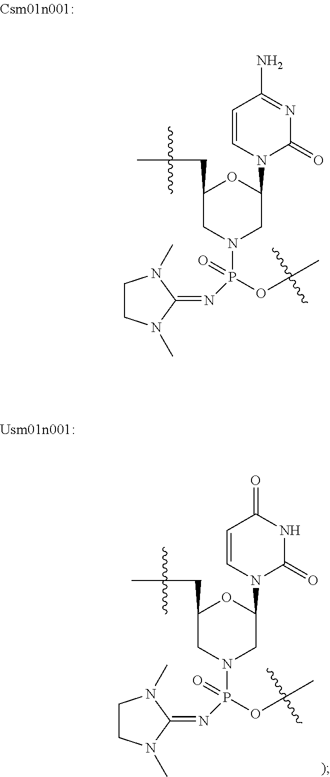 [SMILES: *C[C@@H]1CN(P(=O)(N=C2N(C)CCN2C)O*)C[C@H](n2ccc(N)nc2=O)O1 ; *C[C@@H]1CN(P(=O)(N=C2N(C)CCN2C)O*)C[C@H](n2ccc(=O)[nH]c2=O)O1]

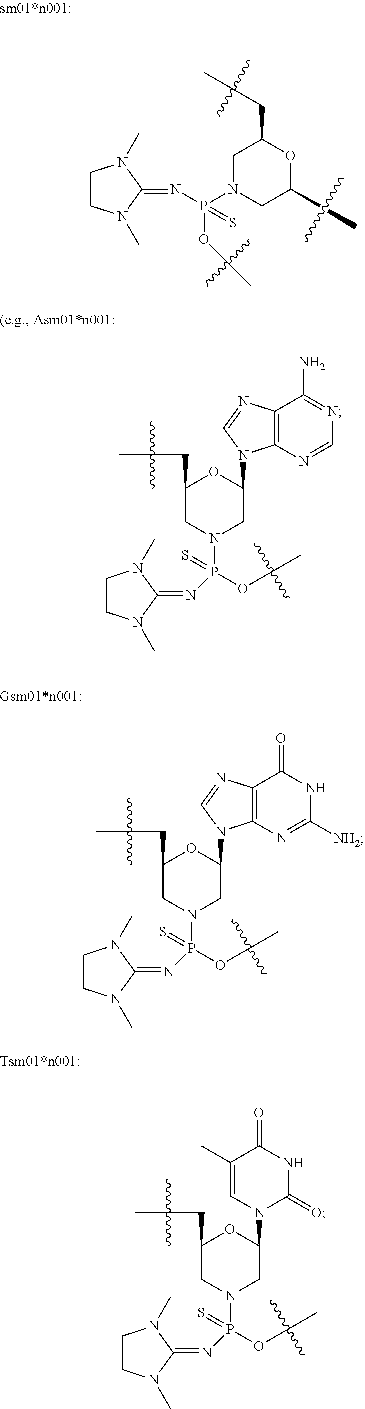

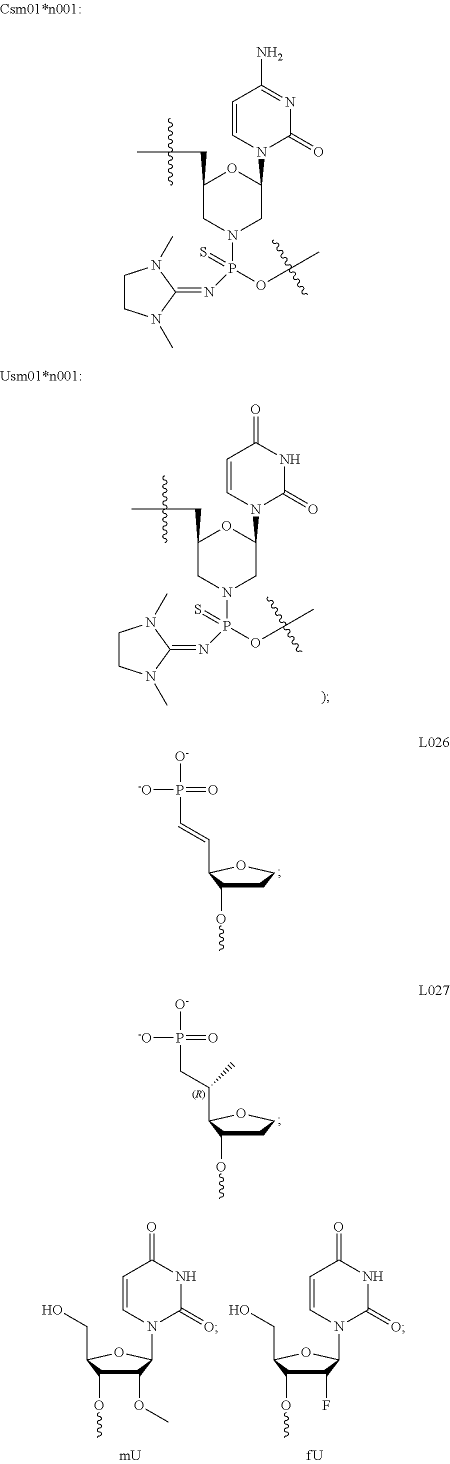

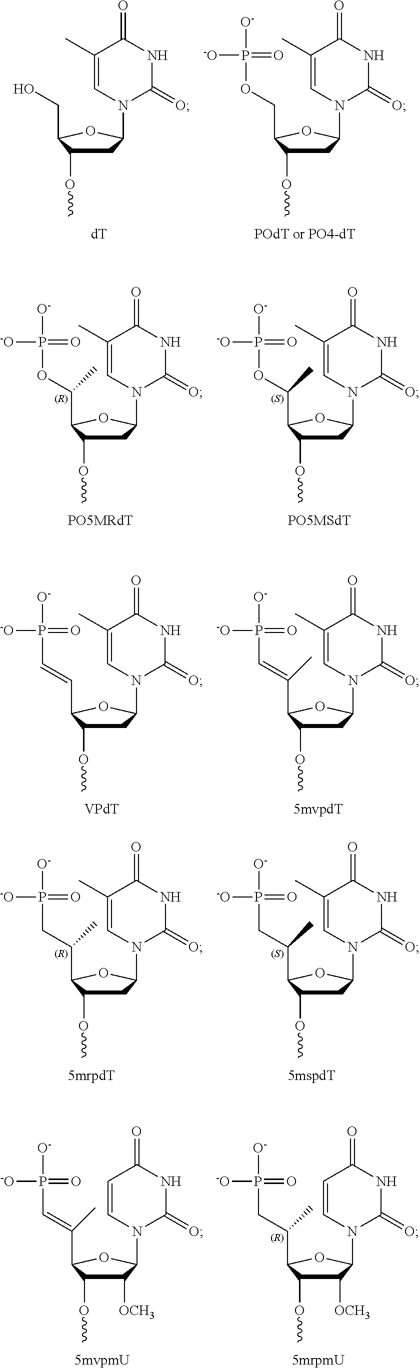

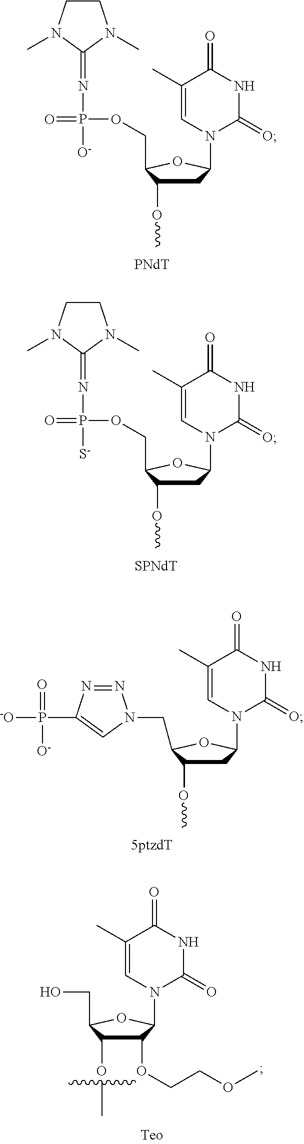

n013:

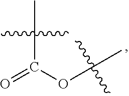 [SMILES: *OC(*)=O]

wherein —C(O)— is bonded to nitrogen;
sm01n013:

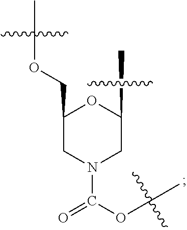

i.e. morpholine carbamate internucleotidic linkage (sm01n013)

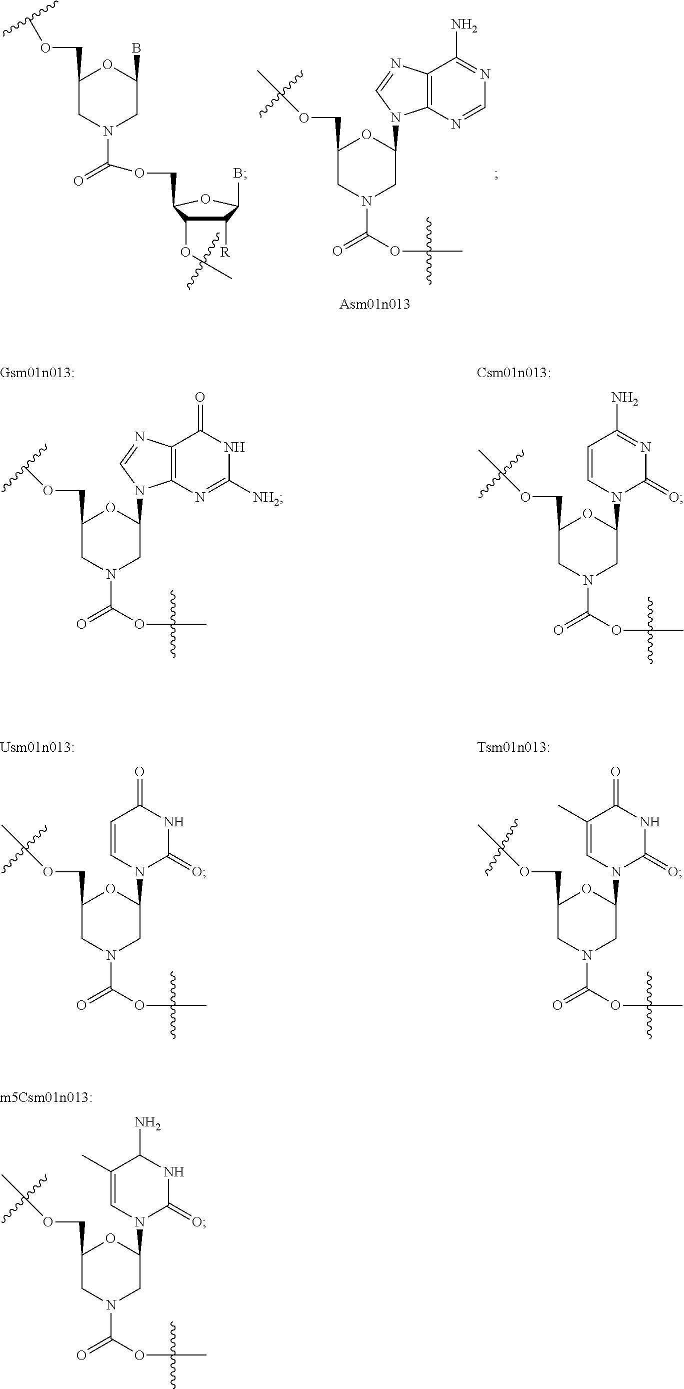

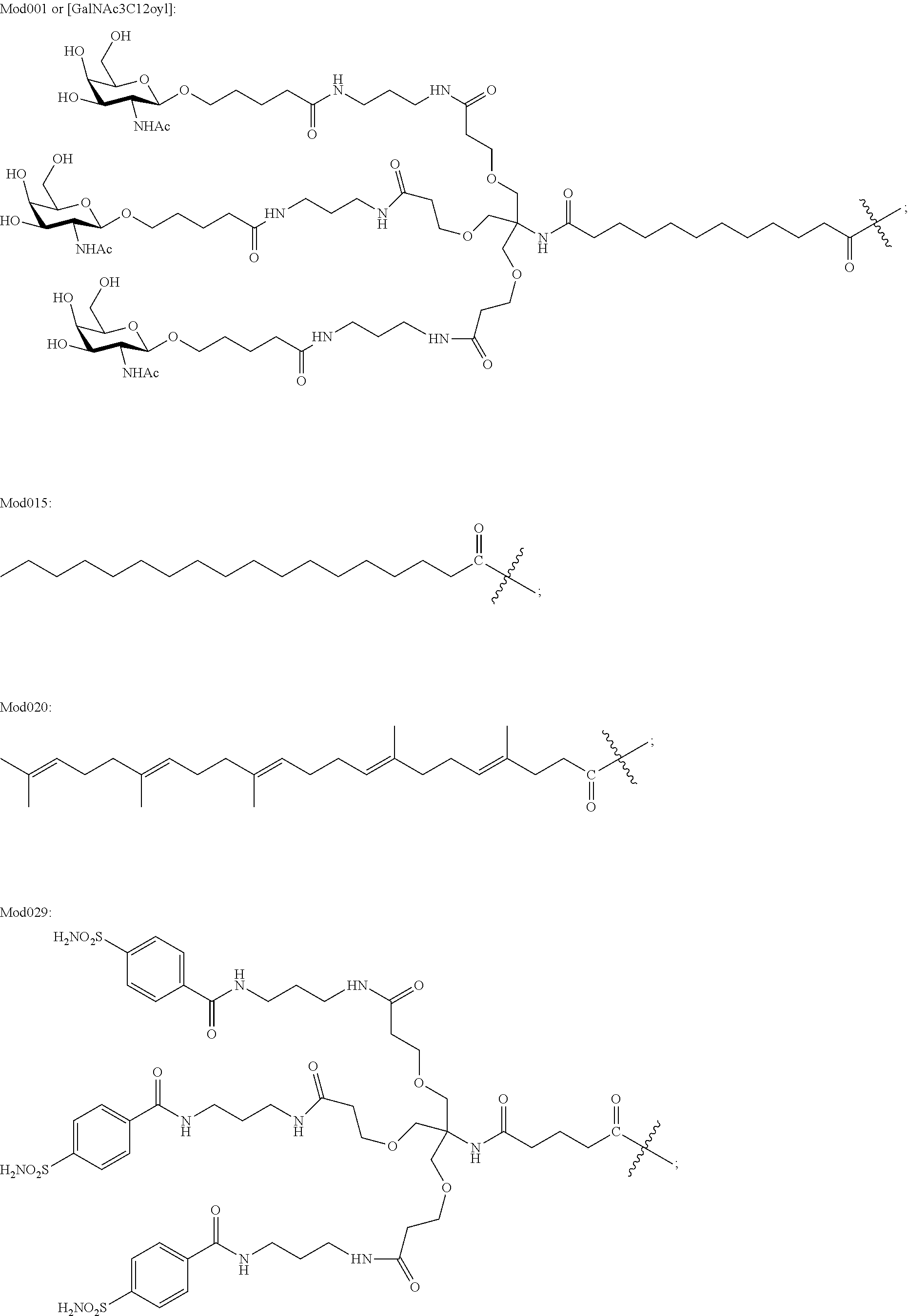

L001 or nC6o: —NH—(CH₂)₆— linker (C₆ linker, C₆ amine linker or C₆ amino linker), connected to Mod (e.g., Mod001) through —NH—, and, in the case of, for example, WV-38061, the 5′-end of the oligonucleotide chain through a phosphate linkage (O or PO). For example, in WV-38061, L001 is connected to Mod001 through —NH— (forming an amide group —C(O)—NH—), and is connected to the oligonucleotide chain through a phosphate linkage (O).

### L010:

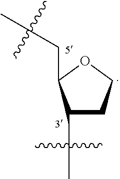

In some embodiments, when L010 is present in the middle of an oligonucleotide, it is bonded to internucleotidic linkages as other sugars (e.g., DNA sugars), e.g., its 5′-carbon is connected to another unit (e.g., 3′ of a sugar) and its 3′-carbon is connected to another unit (e.g., a 5′-carbon of a carbon) independently, e.g., via a linkage (e.g., a phosphate linkage (O or PO) or a phosphorothioate linkage (can be either not chirally controlled or chirally controlled (Sp or Rp)));
L012:—CH₂CH₂OCH₂CH₂OCH₂CH₂—. When L012 is present in the middle of an oligonucleotide, each of its two ends is independently bonded to an internucleotidic linkage (e.g., a phosphate linkage (O or PO) or a phosphorothioate linkage (can be either not chirally controlled or chirally controlled (Sp or Rp)));

### L022:

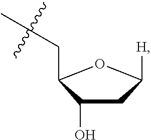

wherein L022 is connected to the rest of a molecule through a phosphate unless indicated otherwise;
L023: HO—(CH₂)₆—, wherein CH₂ is connected to the rest of a molecule through a phosphate unless indicated otherwise. For example, in WV-42644 (wherein the O in OnRnRnRnRSSSSSSSSSSSSSSSSSSnRSSSSSnRSSnR indicates a phosphate linkage connecting L023 to the rest of the molecule);

### L025:

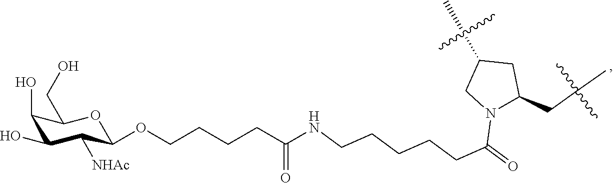

wherein the —CH₂— connection site is utilized as a C5 connection site of a sugar (e.g., a DNA sugar) and is connected to another unit (e.g., 3′ of a sugar), and the connection site on the ring is utilized as a C3 connection site and is connected to another unit (e.g., a 5′-carbon of a carbon), each of which is independently, e.g., via a linkage (e.g., a phosphate linkage (O or PO) or a phosphorothioate linkage (can be either not chirally controlled or chirally controlled (Sp or Rp))). When L025 is at a 5′-end without any modifications, its —CH₂— connection site is bonded to —OH. For example, L025L025L025—in various oligonucleotides has the structure of

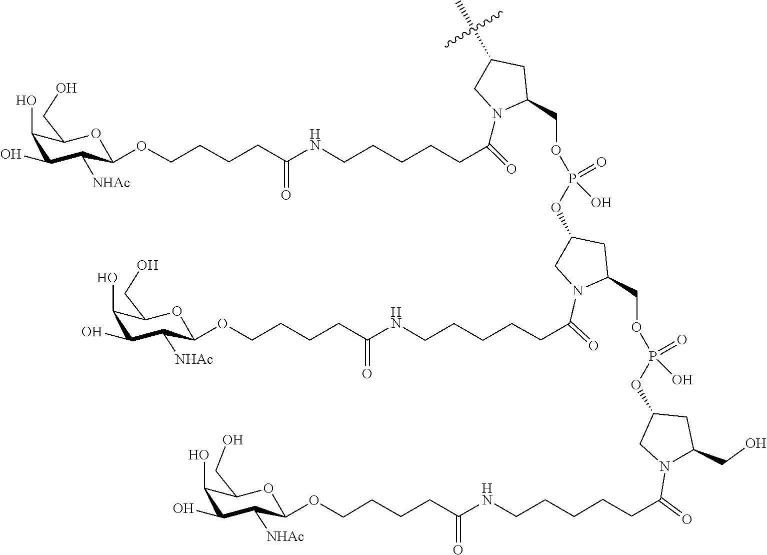

(may exist as various salt forms) and is connected to 5′-carbon of an oligonucleotide chain via a linkage as indicated (e.g., a phosphate linkage (O or PO) or a phosphorothioate linkage (can be either not chirally controlled or chirally controlled (Sp or Rp)));

### L016:

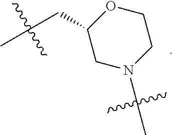

wherein L016 is connected to the rest of a molecule through a phosphate unless indicated otherwise; L016 is utilized with n001 to form L016n001, which has the structure of

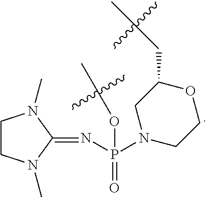 [SMILES: *C[C@H]1CN(P(=O)(N=C2N(C)CCN2C)O*)CCO1]

### 12.2 Double Stranded Oligonucleotide Lengths

As appreciated by those skilled in the art, ds oligonucleotides can be of various lengths to provide desired properties and/or activities for various uses. Many technologies for assessing, selecting and/or optimizing ds oligonucleotide length are available in the art and can be utilized in accordance with the present disclosure. As demonstrated herein, in certain embodiments, dsRNAi oligonucleotides are of suitable lengths to hybridize with their targets and reduce levels of their targets and/or an encoded product thereof. In certain embodiments, a ds oligonucleotide is long enough to recognize a target nucleic acid (e.g., a target mRNA). In certain embodiments, a ds oligonucleotide is sufficiently long to distinguish between a target nucleic acid and other nucleic acids (e.g., a nucleic acid having a base sequence which is not a target sequence) to reduce off-target effects. In certain embodiments, a dsRNAi oligonucleotide is sufficiently short to reduce complexity of manufacture or production and to reduce cost of products.

In certain embodiments, the base sequence of a ds oligonucleotide is about 10-500 nucleobases in length. In certain embodiments, a base sequence is about 10-500 nucleobases in length. In certain embodiments, a base sequence is about 10-50 nucleobases in length. In certain embodiments, a base sequence is about 15-50 nucleobases in length. In certain embodiments, a base sequence is from about 15 to about 30 nucleobases in length. In certain embodiments, a base sequence is from about 10 to about 25 nucleobases in length. In certain embodiments, a base sequence is from about 15 to about 22 nucleobases in length. In certain embodiments, a base sequence is about 10, 11, 12, 13, 14, 15, 16, 17, 18, 19, 20, 21, 22, 23, 24, or 25 nucleobases in length. In certain embodiments, a base sequence is about 18 nucleobases in length. In certain embodiments, a base sequence is about 19 nucleobases in length. In certain embodiments, a base sequence is about 20 nucleobases in length. In certain embodiments, a base sequence is about 21 nucleobases in length. In certain embodiments, a base sequence is about 22 nucleobases in length. In certain embodiments, a base sequence is about 23 nucleobases in length. In certain embodiments, a base sequence is about 24 nucleobases in length. In certain embodiments, a base sequence is about 25 nucleobases in length. In certain embodiments, each nucleobase is optionally substituted A, T, C, G, U, or an optionally substituted tautomer of A, T, C, G, or U.

### 2.2.3. Internucleotidic Linkages

In certain embodiments, ds oligonucleotides comprise base modifications, sugar modifications, and/or internucleotidic linkage modifications. Various internucleotidic linkages can be utilized in accordance with the present disclosure to link units comprising nucleobases, e.g., nucleosides. In certain embodiments, provided ds oligonucleotides comprise both one or more modified internucleotidic linkages and one or more natural phosphate linkages. As widely known by those skilled in the art, natural phosphate linkages are widely found in natural DNA and RNA molecules; they have the structure of —OP(O)(OH)O—, connect sugars in the nucleosides in DNA and RNA, and may be in various salt forms, for example, at physiological pH (about 7.4), natural phosphate linkages are predominantly exist in salt forms with the anion being —OP(O)(O⁻)O—. A modified internucleotidic linkage, or a non-natural phosphate linkage, is an internucleotidic linkage that is not natural phosphate linkage or a salt form thereof. Modified internucleotidic linkages, depending on their structures, may also be in their salt forms. For example, as appreciated by those skilled in the art, phosphorothioate internucleotidic linkages which have the structure of —OP(O)(SH)O— may be in various salt forms, e.g., at physiological pH (about 7.4) with the anion being —OP(O)(S⁻)O—.

In certain embodiments, a ds oligonucleotide comprises an internucleotidic linkage which is a modified internucleotidic linkage, e.g., phosphorothioate, phosphorodithioate, methylphosphonate, phosphoroamidate, thiophosphate, 3′-thiophosphate, or 5′-thiophosphate.

In certain embodiments, a modified internucleotidic linkage is a chiral internucleotidic linkage which comprises a chiral linkage phosphorus. In certain embodiments, a chiral internucleotidic linkage is a phosphorothioate linkage. In certain embodiments, a chiral internucleotidic linkage is a non-negatively charged internucleotidic linkage. In certain embodiments, a chiral internucleotidic linkage is a neutral internucleotidic linkage. In certain embodiments, a chiral internucleotidic linkage is chirally controlled with respect to its chiral linkage phosphorus. In certain embodiments, a chiral internucleotidic linkage is stereochemically pure with respect to its chiral linkage phosphorus. In certain embodiments, a chiral internucleotidic linkage is not chirally controlled. In certain embodiments, a pattern of backbone chiral centers comprises or consists of positions and linkage phosphorus configurations of chirally controlled internucleotidic linkages (Rp or Sp) and positions of achiral internucleotidic linkages (e.g., natural phosphate linkages).

In certain embodiments, an internucleotidic linkage comprises a P-modification, wherein a P-modification is a modification at a linkage phosphorus. In certain embodiments, a modified internucleotidic linkage is a moiety which does not comprise a phosphorus but serves to link two sugars or two moieties that each independently comprises a nucleobase, e.g., as in peptide nucleic acid (PNA).

In certain embodiments, a ds oligonucleotide comprises a modified internucleotidic linkage, e.g., those having the structure of Formula I, I-a, I-b, or I-c and described herein and/or in: WO 2018/022473, WO 2018/098264, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612, the internucleotidic linkages (e.g., those of Formula I, I-a, I-b, I-c, etc.) of each of which are independently incorporated herein by reference. In certain embodiments, a modified internucleotidic linkage is a chiral internucleotidic linkage. In certain embodiments, a modified internucleotidic linkage is a phosphorothioate internucleotidic linkage.

In certain embodiments, a modified internucleotidic linkage is a non-negatively charged internucleotidic linkage. In certain embodiments, provided ds oligonucleotides comprise one or more non-negatively charged internucleotidic linkages. In certain embodiments, a non-negatively charged internucleotidic linkage is a positively charged internucleotidic linkage. In certain embodiments, a non-negatively charged internucleotidic linkage is a neutral internucleotidic linkage. In certain embodiments, the present disclosure provides ds oligonucleotides comprising one or more neutral internucleotidic linkages. In certain embodiments, a non-negatively charged internucleotidic linkage has the structure of Formula I-n-1, I-n-2, I-n-3, I-n-4, II, II-a-1, II-a-2, II-b-1, II-b-2, II-c-1, II-c-2, II-d-1, II-d-2, etc., or a salt form thereof, as described herein and/or in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019/032612, WO 2019/055951, WO 2019/075357, WO 2019/200185, WO 2019/217784, and/or WO 2019/032612, the non-negatively charged internucleotidic linkages (e.g., those of Formula I-n-1, I-n-2, I-n-3, I-n-4, II, II-a-1, II-a-2, II-b-1, II-b-2, II-c-1, II-c-2, II-d-1, II-d-2, etc., or a suitable salt form thereof) of each of which are independently incorporated herein by reference.

In certain embodiments, a non-negatively charged internucleotidic linkage can improve the delivery and/or activities (e.g., adenosine editing activity).

In certain embodiments, a modified internucleotidic linkage (e.g., a non-negatively charged internucleotidic linkage) comprises optionally substituted triazolyl. In certain embodiments, a modified internucleotidic linkage (e.g., a non-negatively charged internucleotidic linkage) comprises optionally substituted alkynyl. In certain embodiments, a modified internucleotidic linkage comprises a triazole or alkyne moiety. In certain embodiments, a triazole moiety, e.g., a triazolyl group, is optionally substituted. In certain embodiments, a triazole moiety, e.g., a triazolyl group) is substituted. In certain embodiments, a triazole moiety is unsubstituted. In certain embodiments, a modified internucleotidic linkage comprises an optionally substituted cyclic guanidine moiety. In certain embodiments, a modified internucleotidic linkage has the structure of

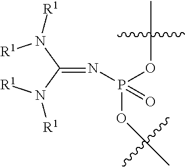

and is optionally chirally controlled, wherein R¹ is -L-R′, wherein L is L^{B} as described herein, and R′ is as described herein. In certain embodiments, each R¹ is independently R′. In certain embodiments, each R′ is independently R. In certain embodiments, two R¹ are R and are taken together to form a ring as described herein. In certain embodiments, two R¹ on two different nitrogen atoms are R and are taken together to form a ring as described herein. In certain embodiments, R¹ is independently optionally substituted C₁₋₆ aliphatic as described herein. In certain embodiments, R¹ is methyl. In certain embodiments, two R′ on the same nitrogen atom are R and are taken together to form a ring as described herein. In certain embodiments, a modified internucleotidic linkage has the structure of

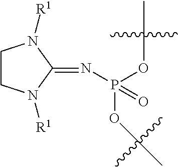

and is optionally chirally controlled. In certain embodiments,

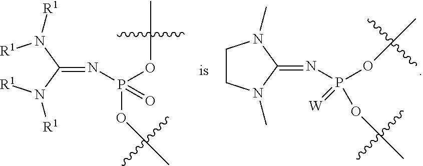

In certain embodiments, a modified internucleotidic linkage comprises an optionally substituted cyclic guanidine moiety and has the structure of:

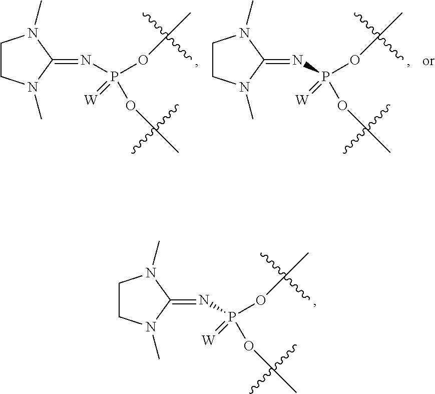

wherein W is O or S. In certain embodiments, W is O. In certain embodiments, W is S. In certain embodiments, a non-negatively charged internucleotidic linkage is stereochemically controlled.

In certain embodiments, a non-negatively charged internucleotidic linkage or a neutral internucleotidic linkage is an internucleotidic linkage comprising a triazole moiety. In some embodiments, an internucleotidic linkage comprising a triazole moiety (e.g., an optionally substituted triazolyl group) has the structure of

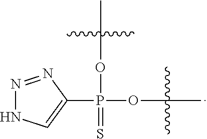 [SMILES: *OP(=S)(O*)c1c[nH]nn1]

In some embodiments, an internucleotidic linkage comprising a triazole moiety has the structure of

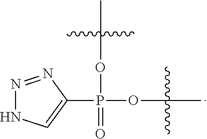 [SMILES: *OP(=O)(O*)c1c[nH]nn1]

In some embodiments, an internucleotidic linkage comprising a triazole moiety has the formula of

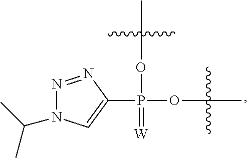

where W is O or S. In some embodiments, an internucleotidic linkage comprising an alkyne moiety (e.g., an optionally substituted alkynyl group) has the formula of

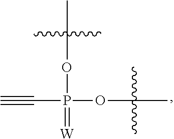

wherein W is O or S. In some embodiments, an internucleotidic linkage, e.g., a non-negatively charged internucleotidic linkage, a neutral internucleotidic linkage, comprises a cyclic guanidine moiety. In some embodiments, an internucleotidic linkage comprising a cyclic guanidine moiety has the structure of

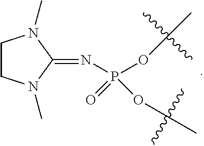 [SMILES: *OP(=O)(N=C1N(C)CCN1C)O*]

In some embodiments, a non-negatively charged internucleotidic linkage, or a neutral internucleotidic linkage, is or comprising a structure selected from

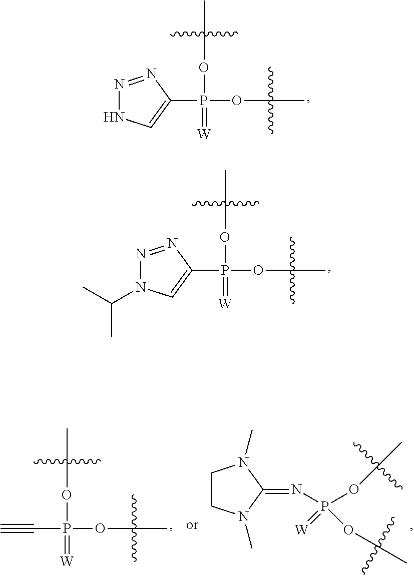

wherein W is O or S. In certain embodiments, an internucleotidic linkage, e.g., a non-negatively charged internucleotidic linkage, a neutral internucleotidic linkage, comprises a cyclic guanidine moiety. In certain embodiments, an internucleotidic linkage comprising a cyclic guanidine moiety has the structure of

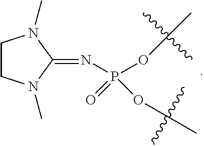 [SMILES: *OP(=O)(N=C1N(C)CCN1C)O*]

In certain embodiments, a non-negatively charged internucleotidic linkage, or a neutral internucleotidic linkage, is or comprising a structure

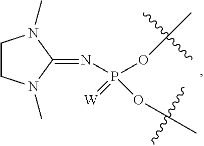

wherein W is O or S.

In certain embodiments, an internucleotidic linkage comprises a Tmg group

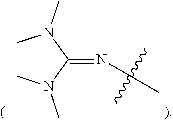 [SMILES: *N=C(N(C)C)N(C)C]

In certain embodiments, an internucleotidic linkage comprises a Tmg group and has the structure of

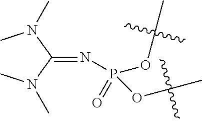 [SMILES: *OP(=O)(N=C(N(C)C)N(C)C)O*]

(the “Tmg internucleotidic linkage”). In certain embodiments, neutral internucleotidic linkages include internucleotidic linkages of PNA and PMO, and a Tmg internucleotidic linkage.

In certain embodiments, a non-negatively charged internucleotidic linkage has the structure of Formula I, I-a, I-b, I-c, I-n-1, I-n-2, I-n-3, I-n-4, II, II-a-1, II-a-2, II-b-1, II-b-2, II-c-1, II-c-2, II-d-1, II-d-2, etc., or a salt form thereof. In certain embodiments, anon-negatively charged internucleotidic linkage comprises an optionally substituted 3-20 membered heterocyclyl or heteroaryl group having 1-10 heteroatoms. In certain embodiments, a non-negatively charged internucleotidic linkage comprises an optionally substituted 3-20 membered heterocyclyl or heteroaryl group having 1-10 heteroatoms, wherein at least one heteroatom is nitrogen. In certain embodiments, such a heterocyclyl or heteroaryl group is of a 5-membered ring. In certain embodiments, such a heterocyclyl or heteroaryl group is of a 6-membered ring.

In certain embodiments, a non-negatively charged internucleotidic linkage comprises an optionally substituted 5-20 membered heteroaryl group having 1-10 heteroatoms. In certain embodiments, a non-negatively charged internucleotidic linkage comprises an optionally substituted 5-20 membered heteroaryl group having 1-10 heteroatoms, wherein at least one heteroatom is nitrogen. In certain embodiments, a non-negatively charged internucleotidic linkage comprises an optionally substituted 5-6 membered heteroaryl group having 1-4 heteroatoms, wherein at least one heteroatom is nitrogen. In certain embodiments, a non-negatively charged internucleotidic linkage comprises an optionally substituted 5-membered heteroaryl group having 1-4 heteroatoms, wherein at least one heteroatom is nitrogen. In certain embodiments, a heteroaryl group is directly bonded to a linkage phosphorus.

In certain embodiments, a non-negatively charged internucleotidic linkage comprises an optionally substituted 5-20 membered heterocyclyl group having 1-10 heteroatoms. In certain embodiments, a non-negatively charged internucleotidic linkage comprises an optionally substituted 5-20 membered heterocyclyl group having 1-10 heteroatoms, wherein at least one heteroatom is nitrogen. In certain embodiments, a non-negatively charged internucleotidic linkage comprises an optionally substituted 5-6 membered heterocyclyl group having 1-4 heteroatoms, wherein at least one heteroatom is nitrogen. In certain embodiments, a non-negatively charged internucleotidic linkage comprises an optionally substituted 5-membered heterocyclyl group having 1-4 heteroatoms, wherein at least one heteroatom is nitrogen. In certain embodiments, at least two heteroatoms are nitrogen. In some embodiments, a non-negatively charged internucleotidic linkage comprises an optionally substituted triazolyl group. In some embodiments, a non-negatively charged internucleotidic linkage comprises an unsubstituted triazolyl group, e.g.,

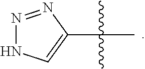 [SMILES: *c1c[nH]nn1]

In some embodiments, a non-negatively charged internucleotidic linkage comprises a substituted triazolyl group, e.g.,

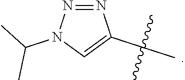 [SMILES: *c1cn(C(C)C)nn1]

In certain embodiments, a heterocyclyl group is directly bonded to a linkage phosphorus. In certain embodiments, a heterocyclyl group is bonded to a linkage phosphorus through a linker, e.g., ═N— when the heterocyclyl group is part of a guanidine moiety who directed bonded to a linkage phosphorus through its ═N−. In certain embodiments, a non-negatively charged internucleotidic linkage comprises an optionally substituted

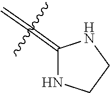 [SMILES: *=C1NCCN1]

group. In certain embodiments, anon-negatively charged internucleotidic linkage comprises an substituted

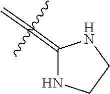 [SMILES: *=C1NCCN1]

group. In certain embodiments, a non-negatively charged internucleotidic linkage comprises a

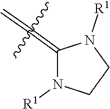

group, wherein each R¹ is independently -L-R. In certain embodiments, each R¹ is independently optionally substituted C₁₋₆ alkyl. In certain embodiments, each R¹ is independently methyl.

In certain embodiments, a modified internucleotidic linkage, e.g., a non-negatively charged internucleotidic linkage, comprises a triazole or alkyne moiety, each of which is optionally substituted. In certain embodiments, a modified internucleotidic linkage comprises a triazole moiety. In certain embodiments, a modified internucleotidic linkage comprises a unsubstituted triazole moiety. In certain embodiments, a modified internucleotidic linkage comprises a substituted triazole moiety. In certain embodiments, a modified internucleotidic linkage comprises an alkyl moiety. In certain embodiments, a modified internucleotidic linkage comprises an optionally substituted alkynyl group. In certain embodiments, a modified internucleotidic linkage comprises an unsubstituted alkynyl group. In certain embodiments, a modified internucleotidic linkage comprises a substituted alkynyl group. In certain embodiments, an alkynyl group is directly bonded to a linkage phosphorus.

In certain embodiments, a ds oligonucleotide comprises different types of internucleotidic phosphorus linkages. In certain embodiments, a chirally controlled oligonucleotide comprises at least one natural phosphate linkage and at least one modified (non-natural) internucleotidic linkage. In certain embodiments, a ds oligonucleotide comprises at least one natural phosphate linkage and at least one phosphorothioate. In certain embodiments, a ds oligonucleotide comprises at least one non-negatively charged internucleotidic linkage. In certain embodiments, a ds oligonucleotide comprises at least one natural phosphate linkage and at least one non-negatively charged internucleotidic linkage. In certain embodiments, a ds oligonucleotide comprises at least one phosphorothioate internucleotidic linkage and at least one non-negatively charged internucleotidic linkage. In certain embodiments, a ds oligonucleotide comprises at least one phosphorothioate internucleotidic linkage, at least one natural phosphate linkage, and at least one non-negatively charged internucleotidic linkage. In certain embodiments, ds oligonucleotides comprise one or more, e.g., 1-50, 1-40, 1-30, 1-20, 1-15, 1-10, 1, 2, 3, 4, 5, 6, 7, 8, 9, 10, 11, 12, 13, 14, 15, 16, 17, 18, 19, 20 or more non-negatively charged internucleotidic linkages. In certain embodiments, a non-negatively charged internucleotidic linkage is not negatively charged in that at a given pH in an aqueous solution less than 50%, 40%, 40%, 30%, 20%, 10%, 5%, or 1% of the internucleotidic linkage exists in a negatively charged salt form. In certain embodiments, a pH is about pH 7.4. In certain embodiments, a pH is about 4-9. In certain embodiments, the percentage is less than 10%. In certain embodiments, the percentage is less than 5%. In certain embodiments, the percentage is less than 1%. In certain embodiments, an internucleotidic linkage is a non-negatively charged internucleotidic linkage in that the neutral form of the internucleotidic linkage has no pKa that is no more than about 1, 2, 3, 4, 5, 6, or 7 in water. In certain embodiments, no pKa is 7 or less. In certain embodiments, no pKa is 6 or less. In certain embodiments, no pKa is 5 or less. In certain embodiments, no pKa is 4 or less. In certain embodiments, no pKa is 3 or less. In certain embodiments, no pKa is 2 or less. In certain embodiments, no pKa is 1 or less. In certain embodiments, pKa of the neutral form of an internucleotidic linkage can be represented by pKa of the neutral form of a compound having the structure of CH₃— the internucleotidic linkage —CH₃. For example, pKa of the neutral form of an internucleotidic linkage having the structure of Formula I may be represented by the pKa of the neutral form of a compound having the structure of

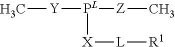

(wherein each of X, Y, Z is independently —O—, —S—, —N(R′)—; L is L^{B}, and R¹ is -L-R′), pKa of

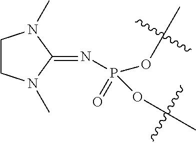 [SMILES: *OP(=O)(N=C1N(C)CCN1C)O*]

can be represented by pKa

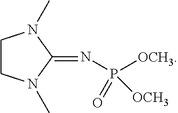

In certain embodiments, a non-negatively charged internucleotidic linkage is a neutral internucleotidic linkage. In certain embodiments, a non-negatively charged internucleotidic linkage is a positively-charged internucleotidic linkage. In certain embodiments, a non-negatively charged internucleotidic linkage comprises a guanidine moiety. In certain embodiments, a non-negatively charged internucleotidic linkage comprises a heteroaryl base moiety. In certain embodiments, a non-negatively charged internucleotidic linkage comprises a triazole moiety. In certain embodiments, a non-negatively charged internucleotidic linkage comprises an alkynyl moiety.

In certain embodiments, a neutral or non-negatively charged internucleotidic linkage has the structure of any neutral or non-negatively charged internucleotidic linkage described in any of U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019/032612, WO 2019/055951, WO 2019/075357, WO 2019/200185, WO 2019/217784, and/or WO 2019/032612,2607, WO2019032612, WO 2019/055951, WO 2019/075357, WO 2019/200185, WO 2019/217784, and/or WO 2019/032612, each neutral or non-negatively charged internucleotidic linkage of each of which is hereby incorporated by reference.

In certain embodiments, each R′ is independently optionally substituted C₁₋₆ aliphatic. In certain embodiments, each R′ is independently optionally substituted C₁₋₆ alkyl. In certain embodiments, each R′ is independently —CH₃. In certain embodiments, each R^{s} is —H.

In certain embodiments, a non-negatively charged internucleotidic linkage has the structure of

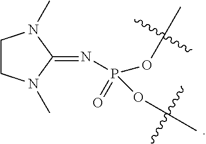 [SMILES: *OP(=O)(N=C1N(C)CCN1C)O*]

In certain embodiments, a non-negatively charged internucleotidic linkage has the structure of

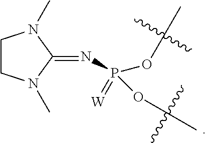

In certain embodiments, a non-negatively charged internucleotidic linkage has the structure of

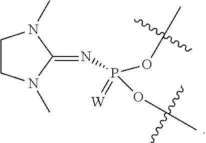

In some embodiments, a non-negatively charged internucleotidic linkage has the structure of

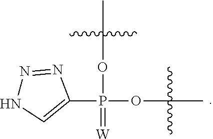

In some embodiments, a non-negatively charged internucleotidic linkage has the structure of

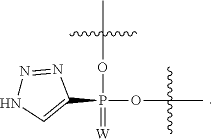

In some embodiments, a non-negatively charged internucleotidic linkage has the structure of

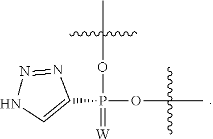

In some embodiments, a non-negatively charged internucleotidic linkage has the structure of

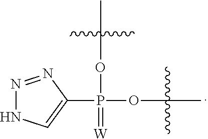

In some embodiments, a non-negatively charged internucleotidic linkage has the structure of

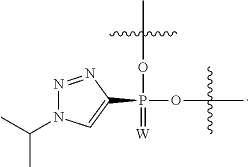

In some embodiments, a non-negatively charged internucleotidic linkage has the structure of

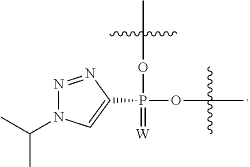

In some embodiments, a non-negatively charged internucleotidic linkage has the structure of

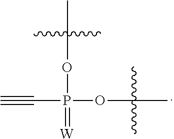

In some embodiments, a non-negatively charged internucleotidic linkage has the structure of

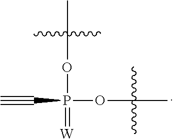

In some embodiments, a non-negatively charged internucleotidic linkage has the structure of

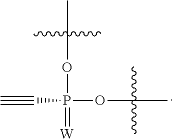

In some embodiments, W is O. In some embodiments, W is S. In some embodiments, a neutral internucleotidic linkage is a non-negatively charged internucleotidic linkage described above.

In certain embodiments, provided ds oligonucleotides comprise 1 or more internucleotidic linkages of Formula I, I-a, I-b, I-c, I-n-1, I-n-2, I-n-3, I-n-4, II, II-a-1, II-a-2, II-b-1, II-b-2, II-c-1, II-c-2, II-d-1, or II-d-2, which are described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019/032612, WO 2019/055951, WO 2019/075357, WO 2019/200185, WO 2019/217784, and/or WO 2019/032612,2607, WO2019032612, WO 2019/055951, WO 2019/075357, WO 2019/200185, WO 2019/217784, and/or WO 2019/032612, the Formula I, I-a, I-b, I-c, I-n-1, I-n-2, I-n-3, I-n-4, II, II-a-1, II-a-2, II-b-1, II-b-2, II-c-1, II-c-2, II-d-1, or II-d-2, or salt forms thereof, each of which are independently incorporated herein by reference.

In certain embodiments, a ds oligonucleotide comprises a neutral internucleotidic linkage and a chirally controlled internucleotidic linkage. In certain embodiments, a ds oligonucleotide comprises a neutral internucleotidic linkage and a chirally controlled internucleotidic linkage which is not the neutral internucleotidic linkage. In certain embodiments, a ds oligonucleotide comprises a neutral internucleotidic linkage and a chirally controlled phosphorothioate internucleotidic linkage. In certain embodiments, the present disclosure provides a ds oligonucleotide comprising one or more non-negatively charged internucleotidic linkages and one or more phosphorothioate internucleotidic linkages, wherein each phosphorothioate internucleotidic linkage in the oligonucleotide is independently a chirally controlled internucleotidic linkage. In certain embodiments, the present disclosure provides a ds oligonucleotide comprising one or more neutral internucleotidic linkages and one or more phosphorothioate internucleotidic linkage, wherein each phosphorothioate internucleotidic linkage in the ds oligonucleotide is independently a chirally controlled internucleotidic linkage. In certain embodiments, a ds oligonucleotide comprises at least 5, 6, 7, 8, 9, 10, 11, 12, 13, 14, 15, 16, 17, 18, 19, 20 or more chirally controlled phosphorothioate internucleotidic linkages. In certain embodiments, non-negatively charged internucleotidic linkage is chirally controlled. In certain embodiments, non-negatively charged internucleotidic linkage is not chirally controlled. In certain embodiments, a neutral internucleotidic linkage is chirally controlled. In certain embodiments, a neutral internucleotidic linkage is not chirally controlled.

Without wishing to be bound by any particular theory, the present disclosure notes that a neutral internucleotidic linkage can be more hydrophobic than a phosphorothioate internucleotidic linkage (PS), which can be more hydrophobic than a natural phosphate linkage (PO). Typically, unlike a PS or PO, a neutral internucleotidic linkage bears less charge. Without wishing to be bound by any particular theory, the present disclosure notes that incorporation of one or more neutral internucleotidic linkages into a ds oligonucleotide may increase the ds oligonucleotides' ability to be taken up by a cell and/or to escape from endosomes. Without wishing to be bound by any particular theory, the present disclosure notes that incorporation of one or more neutral internucleotidic linkages can be utilized to modulate melting temperature of duplexes formed between a ds oligonucleotide and its target nucleic acid.

Without wishing to be bound by any particular theory, the present disclosure notes that incorporation of one or more non-negatively charged internucleotidic linkages, e.g., neutral internucleotidic linkages, into a ds oligonucleotide may be able to increase the ds oligonucleotide's ability to mediate a function such as target adenosine editing.

As appreciated by those skilled in the art, internucleotidic linkages such as natural phosphate linkages and those of Formula I, I-a, I-b, I-c, I-n-1, I-n-2, I-n-3, I-n-4, II, II-a-1, II-a-2, II-b-1, II-b-2, II-c-1, II-c-2, II-d-1, II-d-2, or salt forms thereof typically connect two nucleosides (which can either be natural or modified) as described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019032612, WO 2019/055951, WO 2019/075357, WO 2019/200185, WO 2019/217784, and/or WO 2019/032612, the Formula I, I-a, I-b, I-c, I-n-1, I-n-2, I-n-3, I-n-4, II, II-a-1, II-a-2, II-b-1, II-b-2, II-c-1, II-c-2, II-d-1, II-d-2, or salt forms thereof, each of which are independently incorporated herein by reference. A typical connection, as in natural DNA and RNA, is that an internucleotidic linkage forms bonds with two sugars (which can be either unmodified or modified as described herein). In many embodiments, as exemplified herein an internucleotidic linkage forms bonds through its oxygen atoms or heteroatoms (e.g., Y and Z in various formulae) with one optionally modified ribose or deoxyribose at its 5′ carbon, and the other optionally modified ribose or deoxyribose at its 3′ carbon. In certain embodiments, each nucleoside units connected by an internucleotidic linkage independently comprises a nucleobase which is independently an optionally substituted A, T, C, G, or U, or a substituted tautomer of A, T, C, G or U, or a nucleobase comprising an optionally substituted heterocyclyl and/or a heteroaryl ring having at least one nitrogen atom.

In some embodiments, a linkage has the structure of or comprises —Y—P^{L}(—X—R^{L})—Z—, or a salt form thereof, wherein:
P^{L} is P, P(═W), P—>B(-L^{L}-R^{L})₃, or PN.
W is O, N(-L^{L}-R^{L}), S or Se;
P^{N} is P═N—C(-L^{L}-R′)(=L^{N}-R′) or P═N-L^{L}-R^{L};
L^{N} is ═N-L^{L}-, ═CH-L^{L}- wherein CH is optionally substituted, or ═N⁺(R′)(Q⁻)-L^{L1}-;
Q⁻ is an anion;
each of X, Y and Z is independently —O—, —S—, -L^{L}-N(-L^{L}-R^{L})-L^{L}-, -L^{L}-N═C(-L^{L}-R^{L})-L^{L}-, or L^{L};
each R^{L} is independently -L^{L}-N(R′)₂, -L^{L}-R′, —N═C(-L^{L}-R′)₂, -L^{L}-N(R′)C(NR′)N(R′)₂, -L^{L}-N(R′)C(O)N(R′)₂, a carbohydrate, or one or more additional chemical moieties optionally connected through a linker;
each of L^{L1} and L^{L} is independently L;
-Cy^{IL}- is -Cy-;
each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(NR′)N(R′)—, —N(R′)C(NR′)N(R′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, —OP(OR′)[B(R′)₃]O—, and —[C(R′)₂C(R′)₂O]ₙ—, wherein n is 1-50, and one or more nitrogen or carbon atoms are optionally and independently replaced with Cy^{L};
each -Cy- is independently an optionally substituted bivalent 3-30 membered, monocyclic, bicyclic or polycyclic ring having 0-10 heteroatoms;
each Cy^{L} is independently an optionally substituted trivalent or tetravalent, 3-30 membered, monocyclic, bicyclic or polycyclic ring having 0-10 heteroatoms;
each R′ is independently —R, —C(O)R, —C(O)N(R)₂, —C(O)OR, or —S(O)₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, an internucleotidic linkage has the structure of —O—P^{L}(—X—R^{L})—O—, wherein each variable is independently as described herein. In some embodiments, an internucleotidic linkage has the structure of —O—P(═W)(—X—R^{L})—O—, wherein each variable is independently as described herein. In some embodiments, an internucleotidic linkage has the structure of —O—P(═W)[—N(-L^{L}-R^{L})—R^{L}]—O—, wherein each variable is independently as described herein. In some embodiments, an internucleotidic linkage has the structure of —O—P(═W)(—NH-L^{L}-R^{L})—O—, wherein each variable is independently as described herein. In some embodiments, an internucleotidic linkage has the structure of —O—P(═W)[—N(R′)₂]—O—, wherein each variable is independently as described herein. In some embodiments, an internucleotidic linkage has the structure of —O—P(═W)(—NHR′)—O—, wherein each variable is independently as described herein. In some embodiments, an internucleotidic linkage has the structure of —O—P(═W)(—NHSO₂R)—O—, wherein each variable is independently as described herein. In some embodiments, an internucleotidic linkage has the structure of —O—P(═W)[—N═C(-L^{L}-R′)₂]—O— wherein each variable is independently as described herein. In some embodiments, an internucleotidic linkage has the structure of —O—P(═W)[—N═C[N(R′)₂]₂]—O—, wherein each variable is independently as described herein. In some embodiments, an internucleotidic linkage has the structure of —OP(═W)(—N═C(R″)₂)—O—, wherein each variable is independently as described herein. In some embodiments, an internucleotidic linkage has the structure of —OP(═W)(—N(R″)₂)—O—, wherein each variable is independently as described herein. In some embodiments, W is O. In some embodiments, W is S. In some embodiments, such an internucleotidic linkage is a non-negatively charged internucleotidic linkage. In some embodiments, such an internucleotidic linkage is a neutral internucleotidic linkage.

In some embodiments, an internucleotidic linkage has the structure of —P^{L}(—X—R^{L})—Z—, wherein each variable is independently as described herein. In some embodiments, an internucleotidic linkage has the structure of —P^{L}(—X—R^{L})—O—, wherein each variable is independently as described herein. In some embodiments, an internucleotidic linkage has the structure of —P(═W)(—X—R^{L})—O—, wherein each variable is independently as described herein. In some embodiments, an internucleotidic linkage has the structure of —P(═W)[—N(-L^{L}-R^{L})—R^{L}]—O—, wherein each variable is independently as described herein. In some embodiments, an internucleotidic linkage has the structure of —P(═W)(—NH-L^{L}-R^{L})—O—, wherein each variable is independently as described herein. In some embodiments, an internucleotidic linkage has the structure of —P(═W)[—N(R′)₂]—O—, wherein each variable is independently as described herein. In some embodiments, an internucleotidic linkage has the structure of —P(═W)(—NHR′)—O—, wherein each variable is independently as described herein. In some embodiments, an internucleotidic linkage has the structure of —P(═W)(—NHSO₂R)—O—, wherein each variable is independently as described herein. In some embodiments, an internucleotidic linkage has the structure of —P(═W)[—N═C(-L^{L}-R′)₂]—O—, wherein each variable is independently as described herein. In some embodiments, an internucleotidic linkage has the structure of —P(═W)[—N═C[N(R′)₂]₂]—O—, wherein each variable is independently as described herein. In some embodiments, an internucleotidic linkage has the structure of —P(═W)(—N═C(R″)₂)—O—, wherein each variable is independently as described herein. In some embodiments, an internucleotidic linkage has the structure of —P(═W)(—N(R″)₂)—O—, wherein each variable is independently as described herein. In some embodiments, W is O. In some embodiments, W is S. In some embodiments, such an internucleotidic linkage is a non-negatively charged internucleotidic linkage. In some embodiments, such an internucleotidic linkage is a neutral internucleotidic linkage. In some embodiments, P of such an internucleotidic linkage is bonded to N of a sugar.

In some embodiments, a linkage is a phosphoryl guanidine internucleotidic linkage. In some embodiments, a linkage is a thio-phosphoryl guanidine internucleotidic linkage.

In some embodiments, one or more methylene units are optionally and independently replaced with a moiety as described herein. In some embodiments, L or L^{L} is or comprises —SO₂—. In some embodiments, L or L^{L} is or comprises —SO₂N(R′)—. In some embodiments, L or L^{L} is or comprises —C(O)—. In some embodiments, L or L^{L} is or comprises —C(O)O—. In some embodiments, L or L^{L} is or comprises —C(O)N(R′)—. In some embodiments, L or L^{L} is or comprises —P(═W)(R′)—. In some embodiments, L or L^{L} is or comprises —P(═O)(R′)—. In some embodiments, L or L^{L} is or comprises —P(═S)(R′)—. In some embodiments, L or L^{L} is or comprises —P(R′)—. In some embodiments, L or L^{L} is or comprises —P(═W)(OR′)—. In some embodiments, L or L^{L} is or comprises —P(═O)(OR′)—. In some embodiments, L or L^{L} is or comprises —P(═S)(OR′)—. In some embodiments, L or L^{L} is or comprises —P(OR′)—.

In some embodiments, —X—R^{L} is —N(R′)SO₂R^{L}. In some embodiments, —X—R^{L} is —N(R′)C(O)R^{L}. In some embodiments, —X—R^{L} is N(R′)P(═O)(R′)R^{L}.

In some embodiments, a linkage, e.g., a non-negatively charged internucleotidic linkage or neutral internucleotidic linkage, has the structure of or comprises —P(═W)(—N═C(R″)₂)—, —P(═W)(—N(R′)SO₂R″)—, —P(═W)(—N(R′)C(O)R″)—, —P(═W)(—N(R″)₂)—, —P(═W)(—N(R′)P(O)(R″)₂)—, —OP(═W)(—N═C(R″)₂)O—, —OP(═W)(—N(R′)SO₂R″)O—, —OP(═W)(—N(R′)C(O)R″)O—, —OP(═W)(—N(R″)₂)O—, —OP(═W)(—N(R′)P(O)(R″)₂)O—, —P(═W)(—N═C(R″)₂)O—, —P(═W)(—N(R′)SO₂R″)O—, —P(═W)(—N(R′)C(O)R″)O—, —P(═W)(—N(R″)₂)O—, or —P(═W)(—N(R′)P(O)(R″)₂)O—, or a salt form thereof, wherein:
W is O or S;
each R″ is independently R′, —OR′, —P(═W)(R′)₂, or —N(R′)₂;
each R′ is independently —R, —C(O)R, —C(O)N(R)₂, —C(O)OR, or —S(O)₂R; each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, W is O. In some embodiments, an internucleotidic linkage has the structure of —P(═O)(—N═C(R″)₂)—, —P(═O)(—N(R′)SO₂R″)—, —P(═O)(—N(R′)C(O)R″)—, —P(═O)(—N(R″)₂)—, —P(═O)(—N(R′)P(O)(R″)₂)—, —OP(═O)(—N═C(R″)₂)O—, —OP(═O)(—N(R′)SO₂R″)O—, —OP(═O)(—N(R′)C(O)R″)O—, —OP(═O)(—N(R″)₂)O—, —OP(═O)(—N(R′)P(O)(R″)₂)O—, —P(═O)(—N═C(R″)₂)O—, —P(═O)(—N(R′)SO₂R″)O—, —P(═O)(—N(R′)C(O)R″)O—, —P(═O)(—N(R″)₂)O—, or —P(═O)(—N(R′)P(O)(R″)₂)O—, or a salt form thereof. In some embodiments, an internucleotidic linkage has the structure of —P(═O)(—N═C(R″)₂)—, —P(═O)(—N(R″)₂)—, —OP(═O)(—N═C(R″)₂)—O—, —OP(═O)(—N(R″)₂)—O—, —P(═O)(—N═C(R″)₂)—O— or —P(═O)(—N(R″)₂)—O— or a salt form thereof. In some embodiments, an internucleotidic linkage has the structure of —OP(═O)(—N═C(R″)₂)—O— or —OP(═O)(—N(R″)₂)—O—, or a salt form thereof. In some embodiments, an internucleotidic linkage has the structure of —OP(═O)(—N═C(R″)₂)—O—, or a salt form thereof. In some embodiments, an internucleotidic linkage has the structure of —OP(═O)(—N(R″)₂)—O—, or a salt form thereof. In some embodiments, an internucleotidic linkage has the structure of —OP(═O)(—N(R′)SO₂R″)O—, or a salt form thereof. In some embodiments, an internucleotidic linkage has the structure of —OP(═O)(—N(R′)C(O)R″)O—, or a salt form thereof. In some embodiments, an internucleotidic linkage has the structure of —OP(═O)(—N(R′)P(O)(R″)₂)O—, or a salt form thereof. In some embodiments, a internucleotidic linkage is n001.

In some embodiments, W is S. In some embodiments, an internucleotidic linkage has the structure of —P(═S)(—N═C(R″)₂)—, —P(═S)(—N(R′)SO₂R″)—, —P(═S)(—N(R′)C(O)R″)—, —P(═S)(—N(R″)₂)—, —P(═S)(—N(R′)P(O)(R″)₂)—, —OP(═S)(—N═C(R″)₂)O—, —OP(═S)(—N(R′)SO₂R″)O—, —OP(═S)(—N(R′)C(O)R″)O—, —OP(═S)(—N(R″)₂)O—, —OP(═S)(—N(R′)P(O)(R″)₂)O—, —P(═S)(—N═C(R″)₂)O—, —P(═S)(—N(R′)SO₂R″)O—, —P(═S)(—N(R′)C(O)R″)O—, —P(═S)(—N(R″)₂)O—, or —P(═S)(—N(R′)P(O)(R″)₂)O—, or a salt form thereof. In some embodiments, an internucleotidic linkage has the structure of —P(═S)(—N═C(R″)₂)—, —P(═S)(—N(R″)₂)—, —OP(═S)(—N═C(R″)₂)—O—, —OP(═S)(—N(R″)₂)—O—, —P(═S)(—N═C(R″)₂)—O- or —P(═S)(—N(R″)₂)—O— or a salt form thereof. In some embodiments, an internucleotidic linkage has the structure of —OP(═S)(—N═C(R″)₂)—O— or —OP(═S)(—N(R″)₂)—O—, or a salt form thereof. In some embodiments, an internucleotidic linkage has the structure of —OP(═S)(—N═C(R″)₂)—O—, or a salt form thereof. In some embodiments, an internucleotidic linkage has the structure of —OP(═S)(—N(R″)₂)—O—, or a salt form thereof. In some embodiments, an internucleotidic linkage has the structure of —OP(═S)(—N(R′)SO₂R″)O—, or a salt form thereof. In some embodiments, an internucleotidic linkage has the structure of —OP(═S)(—N(R′)C(O)R″)O—, or a salt form thereof. In some embodiments, an internucleotidic linkage has the structure of —OP(═S)(—N(R′)P(O)(R″)₂)O—, or a salt form thereof. In some embodiments, a internucleotidic linkage is *n001.

In some embodiments, an internucleotidic linkage has the structure of —P(═O)(—N(R′)SO₂R″)—, wherein R″ is as described herein. In some embodiments, an internucleotidic linkage has the structure of —P(═S)(—N(R′)SO₂R″)—, wherein R″ is as described herein. In some embodiments, an internucleotidic linkage has the structure of —P(═O)(—N(R′)SO₂R″)O—, wherein R″ is as described herein. In some embodiments, an internucleotidic linkage has the structure of —P(═S)(—N(R′)SO₂R″)O—, wherein R″ is as described herein. In some embodiments, an internucleotidic linkage has the structure of —OP(═O)(—N(R′)SO₂R″)O—, wherein R″ is as described herein. In some embodiments, an internucleotidic linkage has the structure of —OP(═S)(—N(R′)SO₂R″)O—, wherein R″ is as described herein. In some embodiments, R′, e.g., of —N(R′)—, is hydrogen or optionally substituted C₁0.6 aliphatic. In some embodiments, R′ is C₁₋₆ alkyl. In some embodiments, R′ is hydrogen. In some embodiments, R″, e.g., in —SO₂R″, is R′ as described herein. In some embodiments, an internucleotidic linkage has the structure of —P(═O)(—NHSO₂R″)—, wherein R″ is as described herein. In some embodiments, an internucleotidic linkage has the structure of —P(═S)(—NHSO₂R″)—, wherein R″ is as described herein. In some embodiments, an internucleotidic linkage has the structure of —P(═O)(—NHSO₂R″)O—, wherein R″ is as described herein. In some embodiments, an internucleotidic linkage has the structure of —P(═S)(—NHSO₂R″)O—, wherein R″ is as described herein. In some embodiments, an internucleotidic linkage has the structure of —OP(═O)(—NHSO₂R″)O—, wherein R″ is as described herein. In some embodiments, an internucleotidic linkage has the structure of —OP(═S)(—NHSO₂R″)O—, wherein R″ is as described herein. In some embodiments, —X—R^{L} is —N(R′)SO₂R^{L}, wherein each of R′ and R^{L} is independently as described herein. In some embodiments, R^{L} is R″. In some embodiments, R^{L} is R′. In some embodiments, —X—R^{L} is —N(R′)SO₂R″, wherein R′ is as described herein. In some embodiments, —X—R^{L} is —N(R′)SO₂R′, wherein R′ is as described herein. In some embodiments, —X—R^{L} is —NHSO₂R′, wherein R′ is as described herein. In some embodiments, R′ is R as described herein. In some embodiments, R′ is optionally substituted C₁₋₆ aliphatic. In some embodiments, R′ is optionally substituted C₁₋₆ alkyl. In some embodiments, R′ is optionally substituted phenyl. In some embodiments, R′ is optionally substituted heteroaryl. In some embodiments, R″, e.g., in —SO₂R″, is R. In some embodiments, R is an optionally substituted group selected from C₁₋₆ aliphatic, aryl, heterocyclyl, and heteroaryl. In some embodiments, R is optionally substituted C₁₋₆ aliphatic. In some embodiments, R is optionally substituted C₁₋₆ alkyl. In some embodiments, R is optionally substituted C₁₋₆ alkenyl. In some embodiments, R is optionally substituted C₁₋₆ alkynyl. In some embodiments, R is optionally substituted methyl. In some embodiments, —X—R^{L} is —NHSO₂CH₃. In some embodiments, R is —CF₃. In some embodiments, R is methyl. In some embodiments, R is optionally substituted ethyl. In some embodiments, R is ethyl. In some embodiments, R is —CH₂CHF₂. In some embodiments, R is —CH₂CH₂OCH₃. In some embodiments, R is optionally substituted propyl. In some embodiments, R is optionally substituted butyl. In some embodiments, R is n-butyl. In some embodiments, R is —(CH₂)₆NH₂. In some embodiments, R is an optionally substituted linear C₂₋₂₀ aliphatic. In some embodiments, R is optionally substituted linear C₂₋₂₀ alkyl. In some embodiments, R is linear C₂₋₂₀ alkyl. In some embodiments, R is optionally substituted C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ aliphatic. In some embodiments, R is optionally substituted C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is optionally substituted linear C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is linear C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is optionally substituted phenyl. In some embodiments, R is phenyl. In some embodiments, R is p-methylphenyl. In some embodiments, R is 4-dimethylaminophenyl. In some embodiments, R is 3-pyridinyl. In some embodiments, R is

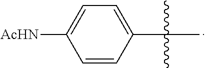 [SMILES: *c1ccc(NC(C)=O)cc1]

In some embodiments, R is

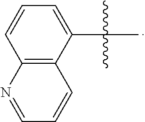 [SMILES: *c1cccc2ncccc12]

In some embodiments, R is benzyl. In some embodiments, R is optionally substituted heteroaryl. In some embodiments, R is optionally substituted 1,3-diazolyl. In some embodiments, R is optionally substituted 2-(1,3)-diazolyl. In some embodiments, R is optionally substituted 1-methyl-2-(1,3)-diazolyl. In some embodiments, R is isopropyl. In some embodiments, R″ is —N(R′)₂. In some embodiments, R″ is —N(CH₃)₂. In some embodiments, R″, e.g., in —SO₂R″, is —OR′, wherein R′ is as described herein. In some embodiments, R′ is R as described herein. In some embodiments, R″ is —OCH₃. In some embodiments, a linkage is —OP(═O)(—NHSO₂R)O—, wherein R is as described herein. In some embodiments, R is optionally substituted linear alkyl as described herein. In some embodiments, R is linear alkyl as described herein. In some embodiments, a linkage is —OP(═O)(—NHSO₂CH₃)O—. In some embodiments, a linkage is —OP(═O)(—NHSO₂CH₂CH₃)O—. In some embodiments, a linkage is —OP(═O)(—NHSO₂CH₂CH₂OCH₃)O—. In some embodiments, a linkage is —OP(═O)(—NHSO₂CH₂Ph)O—. In some embodiments, a linkage is —OP(═O)(—NHSO₂CH₂CHF₂)O—. In some embodiments, a linkage is —OP(═O)(—NHSO₂(4-methylphenyl))O—. In some embodiments, —X—R^{L} is

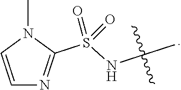 [SMILES: *NS(=O)(=O)c1nccn1C]

In some embodiments, a linkage is —OP(═O)(—X—R^{L})O—, wherein —X—R^{L} is

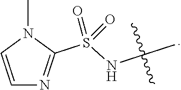 [SMILES: *NS(=O)(=O)c1nccn1C]

In some embodiments, a linkage is —OP(═O)(—NHSO₂CH(CH₃)₂)O—. In some embodiments, a linkage is —OP(═O)(—NHSO₂N(CH₃)₂)O—.

In some embodiments, an internucleotidic linkage has the structure of —P(═O)(—N(R′)C(O)R″)—, wherein R″ is as described herein. In some embodiments, an internucleotidic linkage has the structure of —P(═S)(—N(R′)C(O)R″)—, wherein R″ is as described herein. In some embodiments, an internucleotidic linkage has the structure of —P(═O)(—N(R′)C(O)R″)O—, wherein R″ is as described herein. In some embodiments, an internucleotidic linkage has the structure of —P(═S)(—N(R′)C(O)R″)O—, wherein R″ is as described herein. In some embodiments, an internucleotidic linkage has the structure of —OP(═O)(—N(R′)C(O)R″)O—, wherein R″ is as described herein. In some embodiments, an internucleotidic linkage has the structure of —OP(═S)(—N(R′)C(O)R″)O—, wherein R″ is as described herein. In some embodiments, R′, e.g., of —N(R′)—, is hydrogen or optionally substituted C₁0.6 aliphatic. In some embodiments, R′ is C₁₋₆ alkyl. In some embodiments, R′ is hydrogen. In some embodiments, R″, e.g., in —C(O)R″, is R′ as described herein. In some embodiments, an internucleotidic linkage has the structure of —P(═O)(—NHC(O)R″)—, wherein R″ is as described herein. In some embodiments, an internucleotidic linkage has the structure of —P(═S)(—NHC(O)R″)—, wherein R″ is as described herein. In some embodiments, an internucleotidic linkage has the structure of —P(═O)(—NHC(O)R″)O—, wherein R″ is as described herein. In some embodiments, an internucleotidic linkage has the structure of —P(═S)(—NHC(O)R″)O—, wherein R″ is as described herein. In some embodiments, an internucleotidic linkage has the structure of —OP(═O)(—NHC(O)R″)O—, wherein R″ is as described herein. In some embodiments, an internucleotidic linkage has the structure of —OP(═S)(—NHC(O)R″)O—, wherein R″ is as described herein. In some embodiments, —X—R^{L} is —N(R′)COR^{L}, wherein R^{L} is as described herein. In some embodiments, —X—R^{L} is —N(R′)COR″, wherein R″ is as described herein. In some embodiments, —X—R^{L} is —N(R′)COR′, wherein R′ is as described herein. In some embodiments, —X—R^{L} is —NHCOR′, wherein R′ is as described herein. In some embodiments, R′ is R as described herein. In some embodiments, R′ is optionally substituted C₁₋₆ aliphatic. In some embodiments, R′ is optionally substituted C₁₋₆ alkyl. In some embodiments, R′ is optionally substituted phenyl. In some embodiments, R′ is optionally substituted heteroaryl. In some embodiments, R″, e.g., in —C(O)R″, is R. In some embodiments, R is an optionally substituted group selected from C₁₋₆ aliphatic, aryl, heterocyclyl, and heteroaryl. In some embodiments, R is optionally substituted C₁₋₆ aliphatic. In some embodiments, R is optionally substituted C₁₋₆ alkyl. In some embodiments, R is optionally substituted C₁₋₆ alkenyl. In some embodiments, R is optionally substituted C₁₋₆ alkynyl. In some embodiments, R is methyl. In some embodiments, —X—R^{L} is —NHC(O)CH₃. In some embodiments, R is optionally substituted methyl. In some embodiments, R is —CF₃. In some embodiments, R is optionally substituted ethyl. In some embodiments, R is ethyl. In some embodiments, R is —CH₂CHF₂. In some embodiments, R is —CH₂CH₂OCH₃. In some embodiments, R is optionally substituted C₁₋₂₀ (e.g., C₁₋₆, C₂₋₆, C₃₋₆, C₁₋₁₀, C₂₋₁₀, C₃₋₁₀, C₂₋₂₀, C₃₋₂₀, C₁₀₋₂₀, 1, 2, 3, 4, 5, 6, 7, 8, 9, 10, 11, 12, 13, 14, 15, 16, 17, 18, 19, or 20, etc.) aliphatic. In some embodiments, R is optionally substituted C₁₋₂₀ (e.g., C₁₋₆, C₂₋₆, C₃₋₆, C₁₋₁₀, C₂₋₁₀, C₃₋₁₀, C₂₋₂₀, C₃₋₂₀, C₁₀-20, 1, 2, 3, 4, 5, 6, 7, 8, 9, 10, 11, 12, 13, 14, 15, 16, 17, 18, 19, or 20, etc.) alkyl. In some embodiments, R is an optionally substituted linear C₂₋₂₀ aliphatic. In some embodiments, R is optionally substituted linear C₂₋₂₀ alkyl. In some embodiments, R is linear C₂₋₂₀ alkyl. In some embodiments, R is optionally substituted C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ aliphatic. In some embodiments, R is optionally substituted C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is optionally substituted linear C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is linear C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is optionally substituted aryl. In some embodiments, R is optionally substituted phenyl. In some embodiments, R is p-methylphenyl. In some embodiments, R is benzyl. In some embodiments, R is optionally substituted heteroaryl. In some embodiments, R is optionally substituted 1,3-diazolyl. In some embodiments, R is optionally substituted 2-(1,3)-diazolyl. In some embodiments, R is optionally substituted 1-methyl-2-(1,3)-diazolyl. In some embodiments, R^{L} is —(CH₂)₅NH₂. In some embodiments, R^{L} is

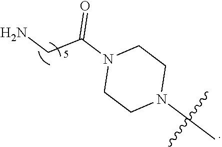

In some embodiments, R^{L} is

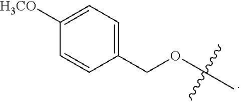 [SMILES: *OCc1ccc(OC)cc1]

In some embodiments, R″ is —N(R′)₂. In some embodiments, R″ is —N(CH₃)₂. In some embodiments, —X—R^{L} is —N(R′)CON(R^{L})₂, wherein each of R′ and R^{L} is independently as described herein. In some embodiments, —X—R^{L} is —NHCON(R^{L})₂, wherein R^{L} is as described herein. In some embodiments, two R′ or two R^{L} are taken together with the nitrogen atom to which they are attached to form a ring as described herein, e.g., optionally substituted

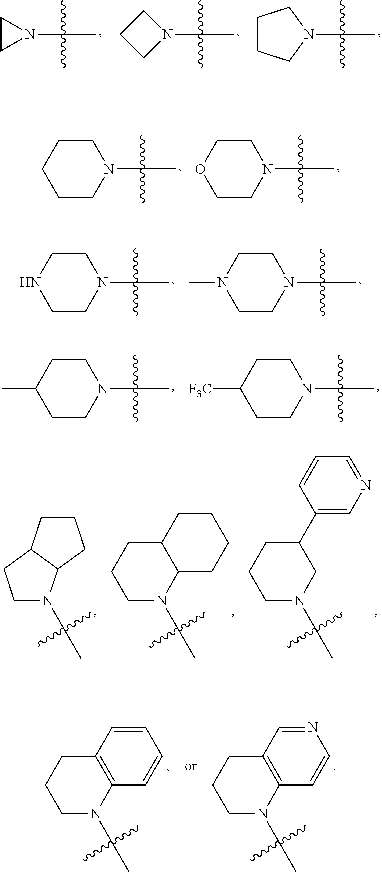

In some embodiments, R″, e.g., in —C(O)R″, is —OR′, wherein R′ is as described herein. In some embodiments, R′ is R as described herein. In some embodiments, is optionally substituted C₁₋₆ aliphatic. In some embodiments, is optionally substituted C₁₋₆ alkyl. In some embodiments, R″ is —OCH₃. In some embodiments, —X—R^{L} is —N(R′)C(O)OR^{L}, wherein each of R′ and R^{L} is independently as described herein. In some embodiments, R is

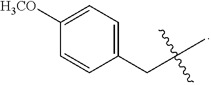 [SMILES: *Cc1ccc(OC)cc1]

In some embodiments, —X—R^{L} is —NHC(O)OCH₃. In some embodiments, —X—R^{L} is —NHC(O)N(CH₃)₂. In some embodiments, a linkage is —OP(O)(NHC(O)CH₃)O—. In some embodiments, a linkage is —OP(O)(NHC(O)OCH₃)O—. In some embodiments, a linkage is —OP(O)(NHC(O)(p-methylphenyl))O—. In some embodiments, a linkage is —OP(O)(NHC(O)N(CH₃)₂)O—. In some embodiments, —X—R^{L} is —N(R′)R^{L}, wherein each of R′ and R^{L} is independently as described herein. In some embodiments, —X—R^{L} is —N(R′)R^{L}, wherein each of R′ and R^{L} is independently not hydrogen. In some embodiments, —X—R^{L}—, —NHR^{L}, wherein R^{L} is as described herein. In some embodiments, R^{L} is not hydrogen. In some embodiments, R^{L} is optionally substituted aryl or heteroaryl. In some embodiments, R^{L} is optionally substituted aryl. In some embodiments, R^{L} is optionally substituted phenyl. In some embodiments, —X—R^{L} is —N(R′)₂, wherein each R′ is independently as described herein. In some embodiments, —X—R^{L} is —NHR′, wherein R′ is as described herein. In some embodiments, —X—R^{L} is —NHR, wherein R is as described herein. In some embodiments, —X—R^{L} is R^{L}, wherein R^{L} is as described herein. In some embodiments, R^{L} is N(R′)₂, wherein each R′ is independently as described herein. In some embodiments, R^{L} is —NHR′, wherein R′ is as described herein. In some embodiments, R^{L} is —NHR, wherein R is as described herein. In some embodiments, R^{L} is —N(R′)₂, wherein each R′ is independently as described herein. In some embodiments, none of R′ in —N(R′)₂ is hydrogen. In some embodiments, R^{L} is —N(R′)₂, wherein each R′ is independently C₁₋₆ aliphatic. In some embodiments, R^{L} is -L-R′, wherein each of L and R′ is independently as described herein. In some embodiments, R^{L} is -L-R, wherein each of L and R is independently as described herein. In some embodiments, R^{L} is —N(R′)—Cy-N(R′)—R′. In some embodiments, R^{L} is —N(R′)—Cy-C(O)—R′. In some embodiments, R^{L} is —N(R′)—Cy-O—R′. In some embodiments, R^{L} is —N(R′)—Cy-SO₂—R′. In some embodiments, R^{L} is —N(R′)—Cy-SO₂—N(R′)₂. In some embodiments, R^{L} is —N(R′)—Cy-C(O)—N(R′)₂. In some embodiments, R^{L} is —N(R′)—Cy-OP(O)(R″)₂. In some embodiments, -Cy- is an optionally substituted bivalent aryl group. In some embodiments, -Cy- is optionally substituted phenylene. In some embodiments, -Cy- is optionally substituted 1,4-phenylene. In some embodiments, -Cy- is 1,4-phenylene. In some embodiments, R^{L} is —N(CH₃)₂. In some embodiments, R^{L} is —N(i-Pr)₂. In some embodiments, R^{L} is

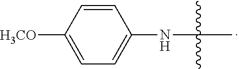 [SMILES: *Nc1ccc(OC)cc1]

In some embodiments, R^{L} is

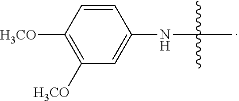 [SMILES: *Nc1ccc(OC)c(OC)c1]

In some embodiments, R^{L} is

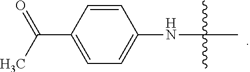 [SMILES: *Nc1ccc(C(C)=O)cc1]

In some embodiments, R^{L} is

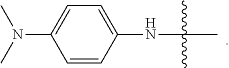 [SMILES: *Nc1ccc(N(C)C)cc1]

In some embodiments, R^{L} is

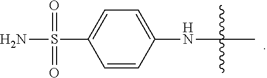 [SMILES: *Nc1ccc(S(N)(=O)=O)cc1]

In some embodiments, R^{L} is

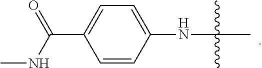 [SMILES: *Nc1ccc(C(=O)NC)cc1]

In some embodiments, R^{L} is

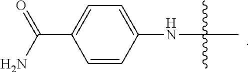 [SMILES: *Nc1ccc(C(N)=O)cc1]

In some embodiments, R^{L} is

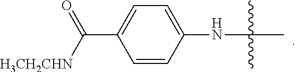

In some embodiments, R^{L} is

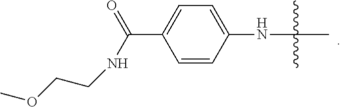 [SMILES: *Nc1ccc(C(=O)NCCOC)cc1]

In some embodiments, R^{L} is

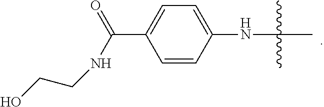 [SMILES: *Nc1ccc(C(=O)NCCO)cc1]

In some embodiments, R^{L} is

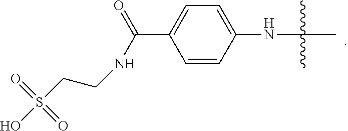 [SMILES: *Nc1ccc(C(=O)NCCS(=O)(=O)O)cc1]

In some embodiments, R^{L} is

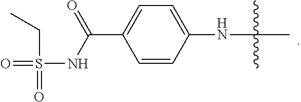 [SMILES: *Nc1ccc(C(=O)NS(=O)(=O)CC)cc1]

In some embodiments, R^{L} is

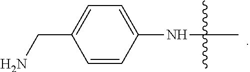 [SMILES: *Nc1ccc(CN)cc1]

In some embodiments, R^{L} is

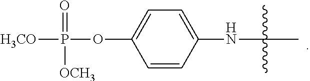 [SMILES: *Nc1ccc(OP(=O)(OC)OC)cc1]

In some embodiments, R^{L} is

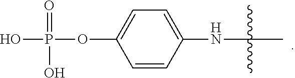 [SMILES: *Nc1ccc(OP(=O)(O)O)cc1]

In some embodiments, R^{L} is

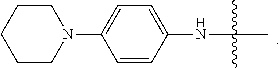 [SMILES: *Nc1ccc(N2CCCCC2)cc1]

In some embodiments, R^{L} is

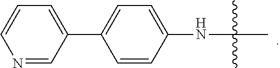 [SMILES: *Nc1ccc(-c2cccnc2)cc1]

In some embodiments, R^{L} is

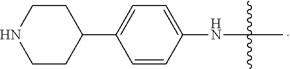 [SMILES: *Nc1ccc(C2CCNCC2)cc1]

In some embodiments, R^{L} is

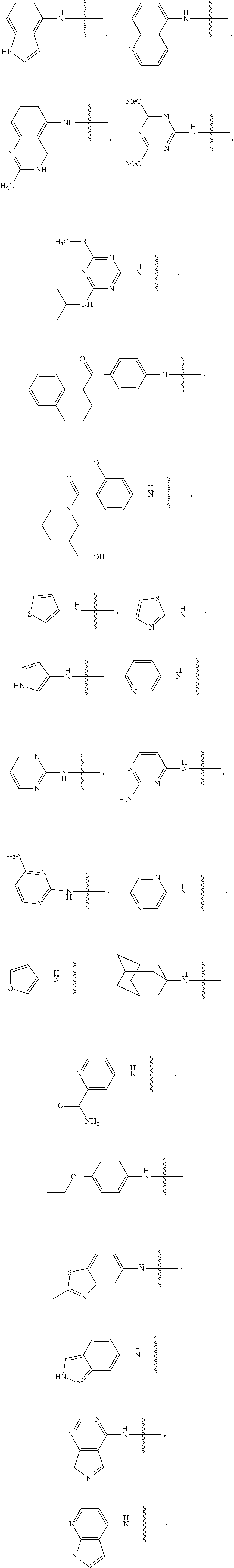

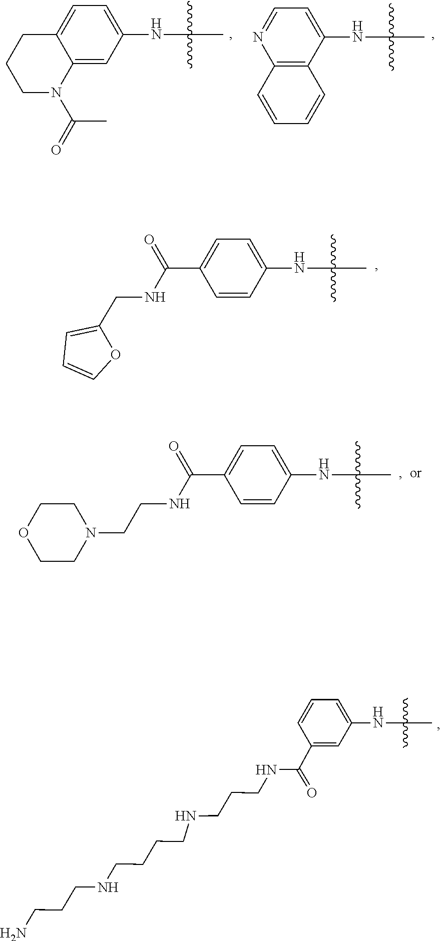 [SMILES: *Nc1ccc2c(c1)N(C(C)=O)CCC2 ; *Nc1ccnc2ccccc12 ; *Nc1ccc(C(=O)NCc2ccco2)cc1 ; *Nc1ccc(C(=O)NCCN2CCOCC2)cc1 ; *Nc1cccc(C(=O)NCCCNCCCCNCCCN)c1]

In some embodiments, —X—R^{L} is —N(R′)—C(O)-Cy-R^{L}. In some embodiments, —X—R^{L} is R^{L}. In some embodiments, R^{L} is —N(R′)—C(O)-Cy-O—R′. In some embodiments, R^{L} is —N(R′)—C(O)-Cy-R′. In some embodiments, R^{L} is —N(R′)—C(O)-Cy-C(O)—R′. In some embodiments, R^{L} is —N(R′)—C(O)-Cy-N(R′)₂. In some embodiments, R^{L} is —N(R′)—C(O)-Cy-SO₂—N(R′)₂. In some embodiments, R^{L} is —N(R′)—C(O)-Cy-C(O)—N(R′)₂. In some embodiments, R^{L} is —N(R′)—C(O)-Cy-C(O)—N(R′)—SO₂—R′. In some embodiments, R′ is R as described herein. In some embodiments, R^{L} is

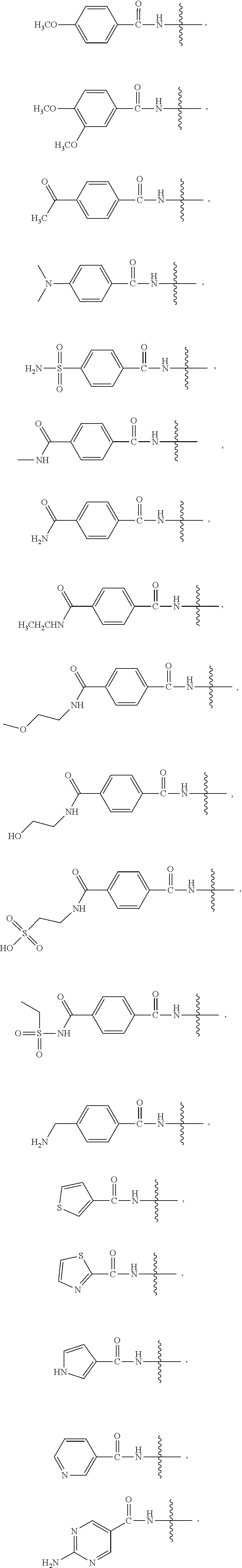

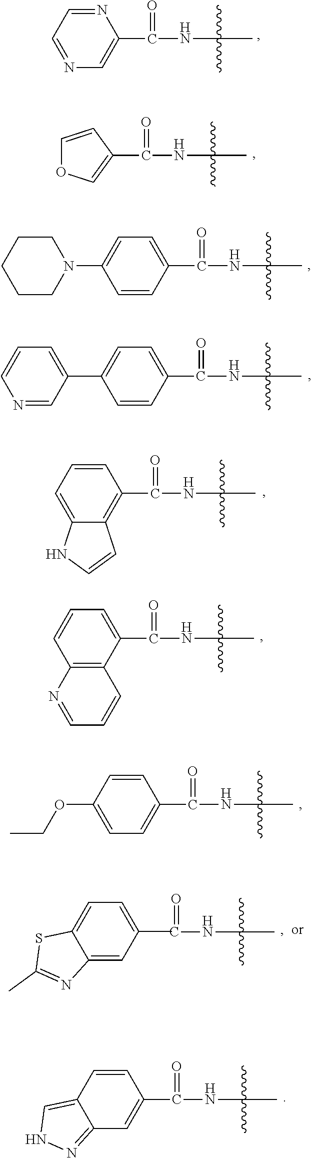

As described herein, in some embodiments, one or more methylene units of L, or a variable which comprises or is L, are independently replaced with —O—, —N(R′)—, —C(O)—, —C(O)N(R′)—, —SO₂—, —SO₂N(R′)—, or -Cy-. In some embodiments, a methylene unit is replaced with -Cy-. In some embodiments, -Cy- is an optionally substituted bivalent aryl group. In some embodiments, -Cy- is optionally substituted phenylene. In some embodiments, -Cy- is optionally substituted 1,4-phenylene. In some embodiments, -Cy- is an optionally substituted bivalent 5-20 (e.g. 5, 6, 7, 8, 9, 10, 11, 12, 13, 14, 15, 16, 17, 18, 19, or 20) membered heteroaryl group having 1-10 (e.g., 1, 2, 3, 4, 5, 6, 7, 8, 9, or 10) heteroatoms. In some embodiments, -Cy- is monocyclic. In some embodiments, -Cy- is bicyclic. In some embodiments, -Cy- is polycyclic. In some embodiments, each monocyclic unit in -Cy- is independently 3-10 (e.g., 3, 4, 5, 6, 7, 8, 9, or 10) membered, and is independently saturated, partially saturated, or aromatic. In some embodiments, -Cy- is an optionally substituted 3-20 (e.g., 3, 4, 5, 6, 7, 8, 9, 10, 11, 12, 13, 14, 15, 16, 17, 18, 19, or 20) membered monocyclic, bicyclic or polycyclic aliphatic group. In some embodiments, -Cy- is an optionally substituted 3-20 (e.g., 3, 4, 5, 6, 7, 8, 9, 10, 11, 12, 13, 14, 15, 16, 17, 18, 19, or 20) membered monocyclic, bicyclic or polycyclic heteroaliphatic group having 1-10 (e.g., 1, 2, 3, 4, 5, 6, 7, 8, 9, or 10) heteroatoms.

In some embodiments, an internucleotidic linkage has the structure of —P(═O)(—N(R′)P(O)(R″)₂)—, wherein each R″ is independently as described herein. In some embodiments, an internucleotidic linkage has the structure of —P(═S)(—N(R′)P(O)(R″)₂)—, wherein each R″ is independently as described herein. In some embodiments, an internucleotidic linkage has the structure of —P(═O)(—N(R′)P(O)(R″)₂)O—, wherein each R″ is independently as described herein. In some embodiments, an internucleotidic linkage has the structure of —P(═S)(—N(R′)P(O)(R″)₂)O—, wherein each R″ is independently as described herein. In some embodiments, an internucleotidic linkage has the structure of —OP(═O)(—N(R′)P(O)(R″)₂)O—, wherein each R″ is independently as described herein. In some embodiments, an internucleotidic linkage has the structure of —OP(═S)(—N(R′)P(O)(R″)₂)O—, wherein each R″ is independently as described herein. In some embodiments, R′, e.g., of —N(R′)—, is hydrogen or optionally substituted C₁₋₆ aliphatic. In some embodiments, R′ is C₁₋₆ alkyl. In some embodiments, R′ is hydrogen. In some embodiments, R″, e.g., in —P(O)(R″)₂, is R′ as described herein. In some embodiments, an internucleotidic linkage has the structure of —P(═O)(—NHP(O)(R″)₂)—, wherein each R″ is independently as described herein. In some embodiments, an internucleotidic linkage has the structure of —P(═S)(—NHP(O)(R″)₂)—, wherein each R″ is independently as described herein. In some embodiments, an internucleotidic linkage has the structure of —P(═O)(—NHP(O)(R″)₂)O—, wherein each R″ is independently as described herein. In some embodiments, an internucleotidic linkage has the structure of —P(═S)(—NHP(O)(R″)₂)O—, wherein each R″ is independently as described herein. In some embodiments, an internucleotidic linkage has the structure of —OP(═O)(—NHP(O)(R″)₂)O—, wherein each R″ is independently as described herein. In some embodiments, an internucleotidic linkage has the structure of —OP(═S)(—NHP(O)(R″)₂)O—, wherein each R″ is independently as described herein. In some embodiments, an occurrence of R″, e.g., in —P(O)(R″)₂, is R. In some embodiments, R is an optionally substituted group selected from C₁₋₆ aliphatic, aryl, heterocyclyl, and heteroaryl. In some embodiments, R is optionally substituted C₁₋₆ aliphatic. In some embodiments, R is optionally substituted C₁₋₆ alkyl. In some embodiments, R is optionally substituted C₁₋₆ alkenyl. In some embodiments, R is optionally substituted C₁₋₆ alkynyl. In some embodiments, R is methyl. In some embodiments, R is optionally substituted methyl. In some embodiments, R is —CF₃. In some embodiments, R is optionally substituted ethyl. In some embodiments, R is ethyl. In some embodiments, R is —CH₂CHF₂. In some embodiments, R is —CH₂CH₂OCH₃. In some embodiments, R is optionally substituted C₁₋₂₀ (e.g., C₁₋₆, C₂₋₆, C₃₋₆, C₁₋₁₀, C₂₋₁₀, C₃₋₁₀, C₂₋₂₀, C₃₋₂₀, C₁₀₋₂₀, 1, 2, 3, 4, 5, 6, 7, 8, 9, 10, 11, 12, 13, 14, 15, 16, 17, 18, 19, or 20, etc.) aliphatic. In some embodiments, R is optionally substituted C₁₋₂₀ (e.g., C₁₋₆, C₂₋₆, C₃₋₆, C₁₋₁₀, C₂₋₁₀, C₃₋₁₀, C₂₋₂₀, C₃₋₂₀, C₁₀₋₂₀, 1, 2, 3, 4, 5, 6, 7, 8, 9, 10, 11, 12, 13, 14, 15, 16, 17, 18, 19, or 20, etc.) alkyl. In some embodiments, R is an optionally substituted linear C₂₋₂₀ aliphatic. In some embodiments, R is optionally substituted linear C₂₋₂₀ alkyl. In some embodiments, R is linear C₂₋₂₀ alkyl. In some embodiments, R is isopropyl. In some embodiments, R is optionally substituted C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ aliphatic. In some embodiments, R is optionally substituted C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is optionally substituted linear C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is linear C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, each R″ is independently R as described herein, for example, in some embodiments, each R″ is methyl. In some embodiments, R″ is optionally substituted aryl. In some embodiments, R is optionally substituted phenyl. In some embodiments, R is p-methylphenyl. In some embodiments, R is benzyl. In some embodiments, R is optionally substituted heteroaryl. In some embodiments, R is optionally substituted 1,3-diazolyl. In some embodiments, R is optionally substituted 2-(1,3)-diazolyl. In some embodiments, R is optionally substituted 1-methyl-2-(1,3)-diazolyl. In some embodiments, an occurrence of R″ is —N(R′)₂. In some embodiments, R″ is —N(CH₃)₂. In some embodiments, an occurrence of R″, e.g., in —P(O)(R″)₂, is —OR′, wherein R′ is as described herein. In some embodiments, R′ is R as described herein. In some embodiments, is optionally substituted C₁₋₆ aliphatic. In some embodiments, is optionally substituted C₁₋₆ alkyl. In some embodiments, R″ is —OCH₃. In some embodiments, each R″ is —OR′ as described herein. In some embodiments, each R″ is —OCH₃. In some embodiments, each R″ is —OH. In some embodiments, a linkage is —OP(O)(NHP(O)(OH)₂)O—. In some embodiments, a linkage is —OP(O)(NHP(O)(OCH₃)₂)O—. In some embodiments, a linkage is —OP(O)(NHP(O)(CH₃)₂)O—.

In some embodiments, —N(R″)₂ is —N(R′)₂. In some embodiments, —N(R″)₂ is —NHR. In some embodiments, —N(R″)₂ is —NHC(O)R. In some embodiments, —N(R″)₂ is —NHC(O)OR. In some embodiments, —N(R″)₂ is —NHS(O)₂R.

In some embodiments, an internucleotidic linkage is a phosphoryl guanidine internucleotidic linkage. In some embodiments, an internucleotidic linkage comprises —X—R^{L} as described herein. In some embodiments, —X—R^{L} is —N═C(-L^{L}-R^{L})₂. In some embodiments, —X—R^{L} is —N═C[N(R^{L})₂]₂. In some embodiments, —X—R^{L} is —N═C[NR′R^{L}]₂. In some embodiments, —X—R^{L} is —N═C[N(R′)₂]₂. In some embodiments, —X—R^{L} is —N═C[N(R^{L})₂](CHR^{L1}R^{L2}), wherein each of R^{L1} and R^{L2} is independently as described herein. In some embodiments, —X—R^{L} is —N═C(NR′R^{L})(CHR^{L1}R^{L2}), wherein each of R^{L1} and R^{L2} is independently as described herein. In some embodiments, —X—R^{L} is N═C(NR′R^{L})(CR′R^{L1}R^{L2}), wherein each of R^{L1} and R^{L2} is independently as described herein. In some embodiments, —X—R^{L} is N═C[N(R′)₂](CHR′R^{L2}). In some embodiments, —X—R^{L} is —N═C[N(R^{L})₂](R^{L}). In some embodiments, —X—R^{L} is —N═C(NR′R^{L})(R^{L}). In some embodiments, —X—R^{L} is N═C(NR′R^{L})(R′). In some embodiments, —X—R^{L} is —N═C[N(R′)₂](R′). In some embodiments, —X—R^{L} is N═C(NR′R^{L1})(NR′R^{L}), wherein each R^{L1} and R^{L2} is independently R^{L}, and each R′ and R^{L} is independently as described herein. In some embodiments, —X—R^{L} is —N═C(NR′R^{L1})(NR′R^{L}), wherein variable is independently as described herein. In some embodiments, —X—R^{L} is N═C(NR′R^{L1})(CHR′R^{L2}), wherein variable is independently as described herein. In some embodiments, —X—R^{L} is N═C(NR′R^{L1})(R′), wherein variable is independently as described herein. In some embodiments, each R′ is independently R. In some embodiments, R is optionally substituted C₁₋₆ aliphatic. In some embodiments, R is methyl. In some embodiments, —X—R^{L} is

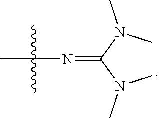 [SMILES: *N=C(N(C)C)N(C)C]

In some embodiments, two groups selected from R′, R^{L}, R^{L1}, R^{L2}, etc. (in some embodiments, on the same atom (e.g., —N(R′)₂, or —NR′R^{L}, or —N(R^{L2} wherein R′ and R^{L} can independently be R as described herein), etc.), or on different atoms (e.g., the two R′ in —N═C(NR′R^{L})(CR′R^{L1}R^{L2}) or —N═C(NR′R^{L1})(NR′R^{L2}); can also be two other variables that can be R, e.g., R^{L}, R^{L1}, R^{L2}, etc.)) are independently R and are taken together with their intervening atoms to form a ring as described herein. In some embodiments, two of R, R′, R^{L}, R^{L1} or R^{L2} on the same atom, e.g., of —N(R′)₂, —N(R^{L})₂, —NR′R^{L}, NR′RY, —NR′R^{L2}, —CR′R^{L1}R^{L2}, etc., are taken together to form a ring as described herein. In some embodiments, two R′, R^{L}, R^{L1}, or R^{L} on two different atoms, e.g., the two R′ in —N═C(NR′R^{L})(CR′R^{L1}R^{L2}), —N═C(NR′R^{L1})(NR′R^{L2}), etc. are taken together to form a ring as described herein. In some embodiments, a formed ring is an optionally substituted 3-20 (e.g., 3-15, 3-12, 3-10, 3-9, 3-8, 3-7, 3-6, 4-15, 4-12, 4-10, 4-9, 4-8, 4-7, 4-6, 5-15, 5-12, 5-10, 5-9, 5-8, 5-7, 5-6, 1,2, 3, 4, 5, 6, 7, 8, 9, 10, 11, 12, 13, 14, 15, 16, 17, 18, 19, 20, etc.) monocyclic, bicyclic or tricyclic ring having 0-5 additional heteroatoms. In some embodiments, a formed ring is monocyclic as described herein. In some embodiments, a formed ring is an optionally substituted 5-10 membered monocyclic ring. In some embodiments, a formed ring is bicyclic. In some embodiments, a formed ring is polycyclic. In some embodiments, two groups that are or can be R (e.g., the two R′ in —N═C(NR′R^{L})(CR′R^{L1}R^{L2}) or —N═C(NR′R^{L1})(NR′R^{L2}), the two R′ in —N═C(NR′R^{L})(CR′R^{L1}R^{L2}), N═C(NR′R^{L1})(NR′R^{L2}), etc.) are taken together to form an optionally substituted bivalent hydrocarbon chain, e.g., an optionally substituted C₁₋₂₀ aliphatic chain, optionally substituted —(CH₂)ₙ— wherein n is 1-20 (e.g., 1, 2, 3, 4, 5, 6, 7, 8, 9, 10, 11, 12, 13, 14, 15, 16, 17, 18, 19, or 20). In some embodiments, a hydrocarbon chain is saturated. In some embodiments, a hydrocarbon chain is partially unsaturated. In some embodiments, a hydrocarbon chain is unsaturated. In some embodiments, two groups that are or can be R (e.g., the two R′ in —N═C(NR′R^{L})(CR′R^{L1}R^{L2}) or —N═C(NR′R^{L1})(NR′R^{L2}), the two R′ in —N═C(NR′R^{L})(CR′R^{L1}R^{L2}), —N═C(NR′R^{L1})(NR′R^{L2}), etc.) are taken together to form an optionally substituted bivalent heteroaliphatic chain, e.g., an optionally substituted C₁₋₂₀ heteroaliphatic chain having 1-10 heteroatoms. In some embodiments, a heteroaliphatic chain is saturated. In some embodiments, a heteroaliphatic chain is partially unsaturated. In some embodiments, a heteroaliphatic chain is unsaturated. In some embodiments, a chain is optionally substituted —(CH₂)—. In some embodiments, a chain is optionally substituted —(CH₂)₂—. In some embodiments, a chain is optionally substituted —(CH₂)—. In some embodiments, a chain is optionally substituted —(CH₂)₂—. In some embodiments, a chain is optionally substituted —(CH₂)₃—. In some embodiments, a chain is optionally substituted —(CH₂)₄—. In some embodiments, a chain is optionally substituted —(CH₂)₅—. In some embodiments, a chain is optionally substituted —(CH₂)₆—. In some embodiments, a chain is optionally substituted —CH═CH—. In some embodiments, a chain is optionally substituted

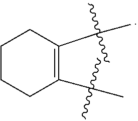 [SMILES: *C1=C(*)CCCC1]

In some embodiments, a chain is optionally substituted

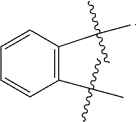 [SMILES: *c1ccccc1*]

In some embodiments, a chain is optionally substituted

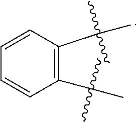 [SMILES: *c1ccccc1*]

In some embodiments, a chain is optionally substituted

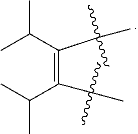 [SMILES: */C(=C(\*)C(C)C)C(C)C]

In some embodiments, a chain is optionally substituted

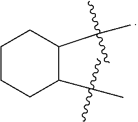 [SMILES: *C1CCCCC1*]

In some embodiments, a chain is optionally substituted

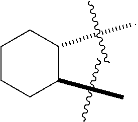 [SMILES: *[C@H]1CCCC[C@@H]1*]

In some embodiments a chain is optionally substituted

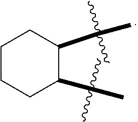 [SMILES: *[C@@H]1CCCC[C@@H]1*]

In some embodiments, a chain is optionally substituted

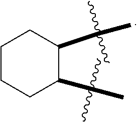 [SMILES: *[C@@H]1CCCC[C@@H]1*]

In some embodiments, a chain is optionally substituted

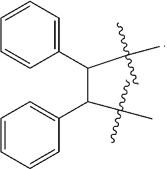 [SMILES: *C(c1ccccc1)C(*)c1ccccc1]

In some embodiments, two of R, R′, R^{L}, R^{L1}, R^{L2}, etc. on different atoms are taken together to form a ring as described herein. For examples, in some embodiments, —X—R^{L} is

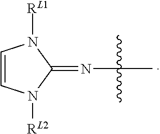

In some embodiments, —X—R^{L} is

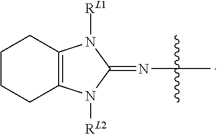

In some embodiments, —X—R^{L} is

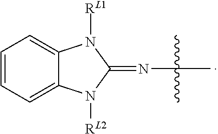

In some embodiments, —X—R^{L} is

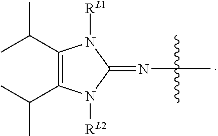

In some embodiments, —X—R^{L} is

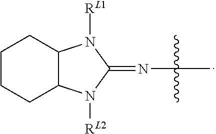

In some embodiments, —X—R^{L} is

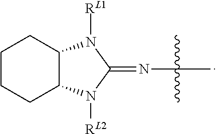

In some embodiments, —X—R^{L} is

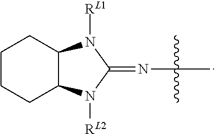

In some embodiments, —X—R^{L} is

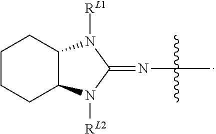

In some embodiments, —X—R^{L} is

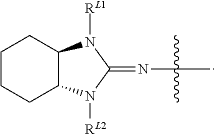

In some embodiments, —X—R^{L} is

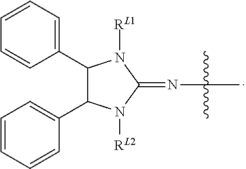

In some embodiments, —N(R′)₂, —N(R)₂, —N(R^{L})₂, —NR′R^{L}, NR′R^{L1}, —NR′R^{L2}, —NR^{L1}R^{L2}, etc. is a formed ring. In some embodiments, a ring is optionally substituted

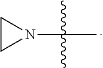 [SMILES: *N1CC1]

In some embodiments, a ring is optionally substituted

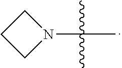 [SMILES: *N1CCC1]

In some embodiments, a ring is optionally substituted

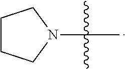 [SMILES: *N1CCCC1]

In some embodiments, a ring is optionally substituted

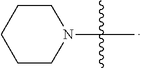 [SMILES: *N1CCCCC1]

In some embodiments, a ring is optionally substituted

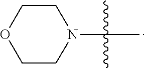 [SMILES: *N1CCOCC1]

In some embodiments, a ring is optionally substituted

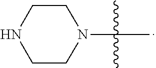 [SMILES: *N1CCNCC1]

In some embodiments, a ring is optionally substituted

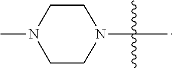 [SMILES: *N1CCN(C)CC1]

In some embodiments, a ring is optionally substituted

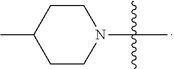 [SMILES: *N1CCC(C)CC1]

In some embodiments, a ring is optionally substituted

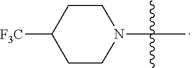 [SMILES: *N1CCC(C(F)(F)F)CC1]

In some embodiments, a ring is optionally substituted

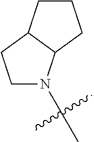 [SMILES: *N1CCC2CCCC21]

In some embodiments, a ring is optionally substituted

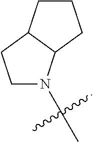 [SMILES: *N1CCC2CCCC21]

In some embodiments, a ring is optionally substituted

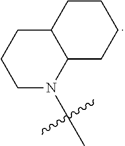 [SMILES: *N1CCCC2CCCCC21]

In some embodiments, a ring is optionally substituted

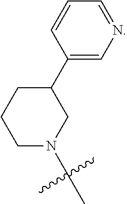 [SMILES: *N1CCCC(c2cccnc2)C1]

In some embodiments, a ring is optionally substituted

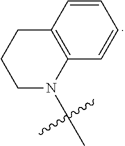 [SMILES: *N1CCCc2ccccc21]

In some embodiments, a ring is optionally substituted

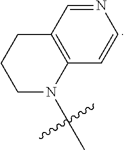 [SMILES: *N1CCCc2cnccc21]

In some embodiments, R^{L1} and R^{L2} are the same. In some embodiments, R^{L1} and R^{L2} are different. In some embodiments, each of R^{L1} and R^{L2} is independently R^{L} as described herein, e.g., below.

In some embodiments, R^{L} is optionally substituted C₁₋₃₀ aliphatic. In some embodiments, R^{L} is optionally substituted C₁₋₃₀ alkyl. In some embodiments, R^{L} is linear. In some embodiments, R^{L} is optionally substituted linear C₁₋₃₀ alkyl. In some embodiments, R^{L} is optionally substituted C₁₋₆ alkyl. In some embodiments, R^{L} is methyl. In some embodiments, R^{L} is ethyl. In some embodiments, R^{L} is n-propyl. In some embodiments, R^{L} is isopropyl. In some embodiments, R^{L} is n-butyl. In some embodiments, R^{L} is tert-butyl. In some embodiments, R^{L} is (E)-CH₂—CH═CH—CH₂—CH₃. In some embodiments R^{L} is (Z)—CH₂—CH═CH—CH₂—CH₃. In some embodiments, R^{L} is

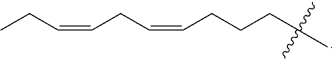 [SMILES: *CCC/C=C\C/C=C\CC]

In some embodiments, R^{L} is

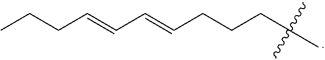 [SMILES: *CCC/C=C/C=C/CCC]

In some embodiments, R^{L} is CH₃(CH₂)₂C≡CC≡C(CH₂)₃—. In some embodiments, R^{L} is CH₃(CH₂)₅C≡C—. In some embodiments, R^{L} optionally substituted aryl. In some embodiments, R^{L} is optionally substituted phenyl. In some embodiments, R^{L} is phenyl substituted with one or more halogen. In some embodiments, R^{L} is phenyl optionally substituted with halogen, —N(R′), or —N(R′)C(O)R′. In some embodiments, R^{L} is phenyl optionally substituted with —Cl, —Br, —F, —N(Me)₂, or —NHCOCH₃. In some embodiments, R^{L} is -L^{L}-R′, wherein L^{L} is an optionally substituted C₁₋₂₀ saturated, partially unsaturated or unsaturated hydrocarbon chain. In some embodiments, such a hydrocarbon chain is linear. In some embodiments, such a hydrocarbon chain is unsubstituted. In some embodiments, L^{L} is (E)-CH₂—CH═CH—. In some embodiments, L^{L} is —CH₂—C≡C—CH₂—. In some embodiments, L^{L} is —(CH₂)₃—. In some embodiments, L^{L} is —(CH₂)₄—. In some embodiments, L^{L} is —(CH₂)ᵣ—, wherein n is 1-30 (e.g., 1-20, 5-30, 6-30, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, etc.). In some embodiments, R′ is optionally substituted aryl as described herein. In some embodiments, R′ is optionally substituted phenyl. In some embodiments, R′ is phenyl. In some embodiments, R′ is optionally substituted heteroaryl as described herein. In some embodiments, R′ is 2′-pyridinyl. In some embodiments, R′ is 3′-pyridinyl. In some embodiments, R^{L} is

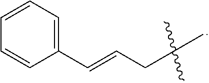 [SMILES: *C/C=C/c1ccccc1]

In some embodiments, R^{L} is

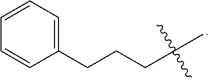 [SMILES: *CCCc1ccccc1]

In some embodiments, R^{L} is

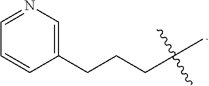 [SMILES: *CCCc1cccnc1]

In some embodiments, R^{L} is -L^{L}-N(R′)₂, wherein each variable is independently as described herein. In some embodiments, each R′ is independently C₁₁-aliphatic as described herein. In some embodiments, —N(R′)₂ is —N(CH₃)₂. In some embodiments, —N(R′)₂ is —NH₂. In some embodiments, R^{L} is —(CH₂)ᵣ—N(R′)₂, wherein n is 1-30 (e.g., 1-20, 5-30, 6-30, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, etc.). In some embodiments, R^{L} is —(CH₂CH₂O)ᵣ—CH₂CH₂—N(R′)₂, wherein n is 1-30 (e.g., 1-20, 5-30, 6-30, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, etc.). In some embodiments, R^{L} is

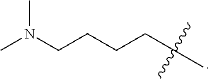 [SMILES: *CCCCN(C)C]

In some embodiments, R^{L} is

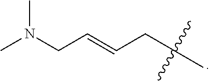 [SMILES: *C/C=C/CN(C)C]

In some embodiments, R^{L} is

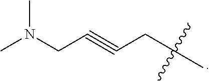 [SMILES: *CC#CCN(C)C]

In some embodiments, R^{L} is —(CH₂)—NH₂. In some embodiments, R^{L} is —(CH₂CH₂O)ᵣ—CH₂CH₂—NH₂. In some embodiments, R^{L} is —(CH₂CH₂O)ᵣ—CH₂CH₂—R′, wherein n is 1-30 (e.g., 1-20, 5-30, 6-30, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, etc.). In some embodiments, R^{L} is —(CH₂CH₂O)ₙ—CH₂CH₂CH₃, wherein n is 1-30 (e.g., 1-20, 5-30, 6-30, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, etc.). In some embodiments, R^{L} is —(CH₂CH₂O)ᵣ—CH₂CH₂OH, wherein n is 1-30 (e.g., 1-20, 5-30, 6-30, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, etc.). In some embodiments, R^{L} is or comprises a carbohydrate moiety, e.g., GalNAc. In some embodiments, R^{L} is -L^{L}-GalNAc. In some embodiments, R^{L} is

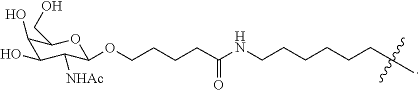 [SMILES: *CCCCCCNC(=O)CCCCOC1OC(CO)C(O)C(O)C1NC(C)=O]

In some embodiments, one or more methylene units of L^{L} are independently replaced with -Cy- (e.g., optionally substituted 1,4-phenylene, a 3-30 membered bivalent optionally substituted monocyclic, bicyclic, or polycyclic cycloaliphatic ring, etc.), —O—, —N(R′)— (e.g., —NH), —C(O)—, —C(O)N(R′)— (e.g., —C(O)NH—), —C(NR′)— (e.g., —C(NH)—), —N(R′)C(O)(N(R′)— (e.g., —NHC(O)NH—), —N(R′)C(NR′)(N(R′)— (e.g., —NHC(NH)NH—), —(CH₂CH₂O)ₙ—, etc. For example, in some embodiments, R^{L} is

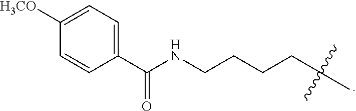 [SMILES: *CCCCNC(=O)c1ccc(OC)cc1]

In some embodiments, R^{L} is

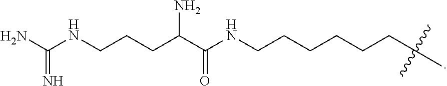 [SMILES: *CCCCCCNC(=O)C(N)CCCNC(=N)N]

In some embodiments, R^{L} is

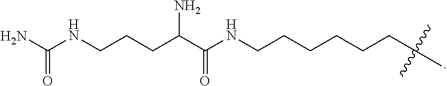 [SMILES: *CCCCCCNC(=O)C(N)CCCNC(N)=O]

In some embodiments, R^{L} is

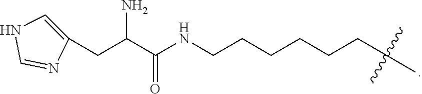 [SMILES: *CCCCCCNC(=O)C(N)Cc1c[nH]cn1]

In some embodiments, R^{L} is

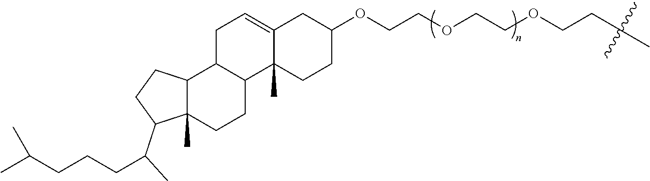

wherein n is 0-20. In some embodiments, R^{L} is or comprises one or more additional chemical moieties (e.g., carbohydrate moieties, GalNAc moieties, etc.) optionally substituted connected through a linker (which can be bivalent or polyvalent). For example, in some embodiments, R^{L} is

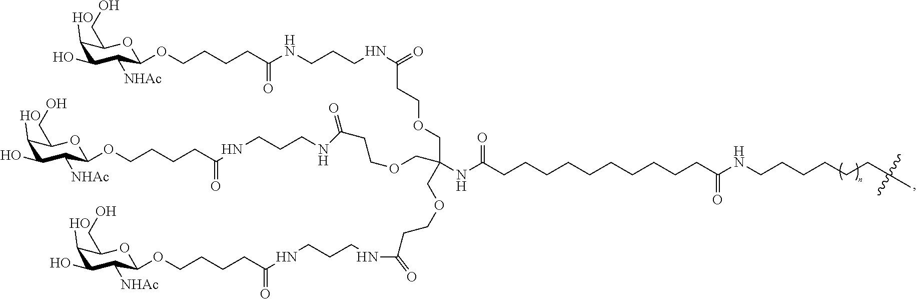

wherein n is 0-20. In some embodiments, R^{L} is

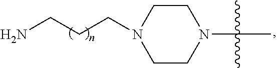

wherein n is 0-20. In some embodiments, R^{L} is R′ as described herein. As described herein, many variable can independently be R′. In some embodiments, R′ is R as described herein. As described herein, various variables can independently be R. In some embodiments, R is optionally substituted C₁₋₆ aliphatic. In some embodiments, R is optionally substituted C₁₋₆ alkyl. In some embodiments, R is methyl. In some embodiments, R is optionally substituted cycloaliphatic. In some embodiments, R is optionally substituted cycloalkyl. In some embodiments, R is optionally substituted aryl. In some embodiments, R is optionally substituted phenyl. In some embodiments, R is optionally substituted heteroaryl. In some embodiments, R is optionally substituted heterocyclyl. In some embodiments, R is optionally substituted C₁₋₂₀ heterocyclyl having 1-5 heteroatoms, e.g., one of which is nitrogen. In some embodiments, R is optionally substituted

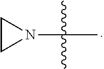 [SMILES: *N1CC1]

In some embodiments, R is optionally substituted

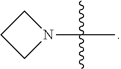 [SMILES: *N1CCC1]

In some embodiments, R is optionally substituted

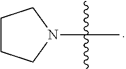 [SMILES: *N1CCCC1]

In some embodiments, R is optionally substituted

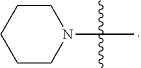 [SMILES: *N1CCCCC1]

In some embodiments, R is optionally substituted

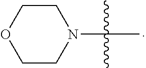 [SMILES: *N1CCOCC1]

In some embodiments, R is optionally substituted

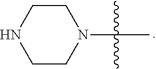 [SMILES: *N1CCNCC1]

In some embodiments, R is optionally substituted

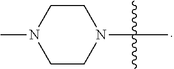 [SMILES: *N1CCN(C)CC1]

In some embodiments, R is optionally substituted

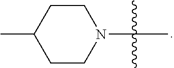 [SMILES: *N1CCC(C)CC1]

In some embodiments, R is optionally substituted

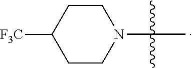 [SMILES: *N1CCC(C(F)(F)F)CC1]

In some embodiments, R is optionally substituted

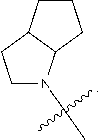 [SMILES: *N1CCC2CCCC21]

In some embodiments, R is optionally substituted

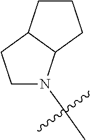 [SMILES: *N1CCC2CCCC21]

In some embodiments, R is optionally substituted

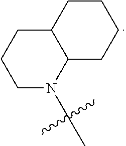 [SMILES: *N1CCCC2CCCCC21]

In some embodiments, R is optionally substituted

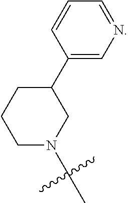 [SMILES: *N1CCCC(c2cccnc2)C1]

In some embodiments, R is optionally substituted

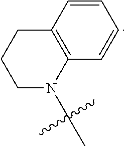 [SMILES: *N1CCCc2ccccc21]

In some embodiments, R is optionally substituted

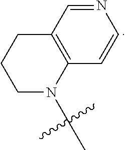 [SMILES: *N1CCCc2cnccc21]

In some embodiments, —X—R^{L} is

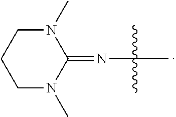 [SMILES: *N=C1N(C)CCCN1C]

In some embodiments, —X—R^{L} is

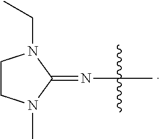 [SMILES: *N=C1N(C)CCN1CC]

In some embodiments, —X—R^{L} is

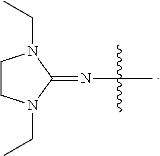 [SMILES: *N=C1N(CC)CCN1CC]

In some embodiments, —X—R^{L} is

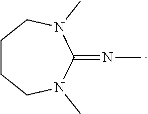

In some embodiments, —X—R^{L} is

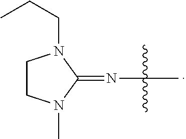 [SMILES: *N=C1N(C)CCN1CCC]

In some embodiments, —X—R^{L} is

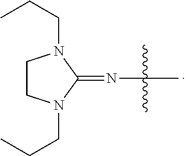 [SMILES: *N=C1N(CCC)CCN1CCC]

In some embodiments, —X—R^{L} is

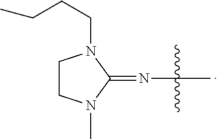 [SMILES: *N=C1N(C)CCN1CCCC]

In some embodiments, —X—R^{L} is

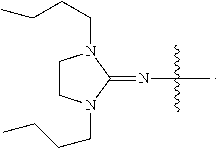 [SMILES: *N=C1N(CCCC)CCN1CCCC]

In some embodiments, —X—R^{L} is

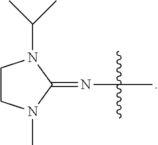 [SMILES: *N=C1N(C)CCN1C(C)C]

In some embodiments, —X—R^{L} is

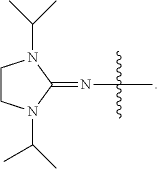 [SMILES: *N=C1N(C(C)C)CCN1C(C)C]

In some embodiments, —X—R^{L} is

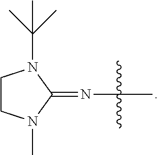 [SMILES: *N=C1N(C)CCN1C(C)(C)C]

In some embodiments, —X—R^{L} s

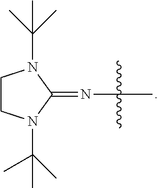 [SMILES: *N=C1N(C(C)(C)C)CCN1C(C)(C)C]

In some embodiments, —X—R^{L} is

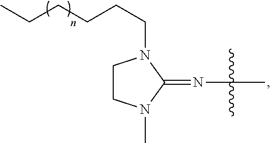

wherein n is 1-20. In some embodiments, —X—R^{L} is

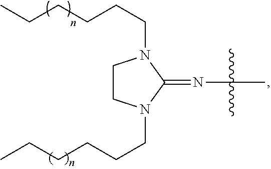

wherein n is 1-20. In some embodiments, —X—R^{L} is selected from:

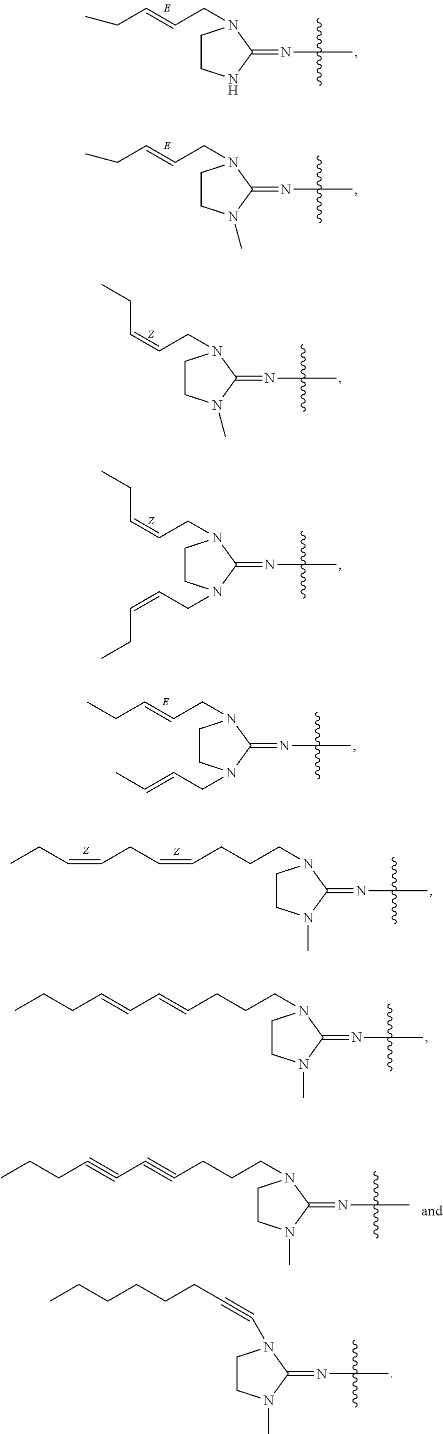

In some embodiments, —X—R^{L} is

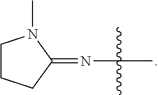 [SMILES: *N=C1CCCN1C]

In some embodiments, —X—R^{L} is

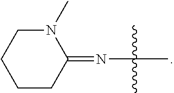 [SMILES: *N=C1CCCCN1C]

In some embodiments, —X—R^{L} is

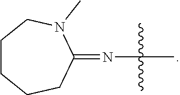 [SMILES: *N=C1CCCCCN1C]

In some embodiments, R^{L} is R″ as described herein. In some embodiments, R^{L} is R as described herein.

In some embodiments, R″ or R^{L} is or comprises an additional chemical moiety. In some embodiments, R″ or R^{L} is or comprises an additional chemical moiety, wherein the additional chemical moiety is or comprises a carbohydrate moiety. In some embodiments, R″ or R^{L} is or comprises a GalNAc. In some embodiments, R^{L} or R″ is replaced with, or is utilized to connect to, an additional chemical moiety.

In some embodiments, X is —O—. In some embodiments, X is —S—. In some embodiments, X is -L^{L}-N(-L^{L}-R^{L})-L^{L}-. In some embodiments, X is —N(-L^{L}-R^{L})-L^{L}-. In some embodiments, X is -L^{L}-N(-L^{L}-R^{L})_. In some embodiments, X is —N(-L^{L}-R^{L})_. In some embodiments, X is -L^{L}-N═C(-L^{L}-R^{L})-L^{L}-. In some embodiments, X is —N═C(-L^{L}-R^{L})L^{L} In some embodiments, X is -L^{L}-N═C(-L^{L}-R^{L})_. In some embodiments, X is —N═C(-L^{L}-R^{L})—. In some embodiments, X is L^{L}. In some embodiments, X is a covalent bond.

In some embodiments, Y is a covalent bond. In some embodiments, Y is —O—. In some embodiments, Y is —N(R′)—. In some embodiments, Z is a covalent bond. In some embodiments, Z is —O—. In some embodiments, Z is —N(R′)—. In some embodiments, R′ is R. In some embodiments, R is —H. In some embodiments, R is optionally substituted C₁₋₆ aliphatic. In some embodiments, R is methyl. In some embodiments, R is ethyl. In some embodiments, R is propyl. In some embodiments, R is optionally substituted phenyl. In some embodiments, R is phenyl.

As described herein, various variables in structures in the present disclosure can be or comprise R. Suitable embodiments for R are described extensively in the present disclosure. As appreciated by those skilled in the art, R embodiments described for a variable that can be R may also be applicable to another variable that can be R. Similarly, embodiments described for a component/moiety (e.g., L) for a variable may also be applicable to other variables that can be or comprise the component/moiety.

In some embodiments, R″ is R′. In some embodiments, R″ is —N(R′)₂.

In some embodiments, —X—R^{L} is —SH. In some embodiments, —X—R^{L} is —OH.

In some embodiments, —X—R^{L} is —N(R′)₂. In some embodiments, each R′ is independently optionally substituted C₁₋₆ aliphatic. In some embodiments, each R′ is independently methyl.

In some embodiments, a non-negatively charged internucleotidic linkage has the structure of —OP(═O)(—N═C((N(R′)₂)₂—O—. In some embodiments, a R′ group of one N(R′)₂ is R, a R′ group of the other N(R′)₂ is R, and the two R groups are taken together with their intervening atoms to form an optionally substituted ring, e.g., a 5-membered ring as in n001. In some embodiments, each R′ is independently R, wherein each R is independently optionally substituted C₁₋₆ aliphatic.

In some embodiments, —X—R^{L} is —N═C(-L^{L}-R′)₂. In some embodiments, —X—R^{L} is —N═C(-L^{L1}-L^{L2}-L^{L3}-R′)₂, wherein each L^{L1}, L^{L2} and L^{L3} is independently L″, wherein each L″ is independently a covalent bond, or a bivalent, optionally substituted, linear or branched group selected from a C₁₋₁₀ aliphatic group and a C₁₋₁₀ heteroaliphatic group having 1-5 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L}. In some embodiments, L^{L2} is -Cy-. In some embodiments, L^{L1} is a covalent bond. In some embodiments, L^{L3} is a covalent bond. In some embodiments, —X—R^{L} is —N═C(-L^{L1}-Cy-L^{L3}-R′)₂. In some embodiments, —X—R^{L} is [symbol] . In some embodiments, —X—R^{L} is [symbol] . In some embodiments, —X—R^{L1}. In some embodiments, —X—R^{L} is [symbol] . In some embodiments, —X—R^{L} is [symbol] . In some embodiments, —X—R^{L} is [symbol] .

In some embodiments, as utilized in the present disclosure, L is covalent bond. In some embodiments, L is a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L}. In some embodiments, L is a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L}. In some embodiments, L is a bivalent, optionally substituted, linear or branched group selected from a C₁₋₁₀ aliphatic group and a C₁₋₁₀ heteroaliphatic group having 1-10 heteroatoms, wherein one or more methylene units are optionally and independently replaced by an optionally substituted group selected from —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L}. In some embodiments, one or more methylene units are optionally and independently replaced by an optionally substituted group selected from —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.

In some embodiments, an internucleotidic linkage is a phosphoryl guanidine internucleotidic linkage. In some embodiments, —X—R^{L} is —N═C[N(R′)₂]₂. In some embodiments, each R′ is independently R. In some embodiments, R is optionally substituted C₁₋₆ aliphatic. In some embodiments, R is methyl. In some embodiments, —X—R^{L} is

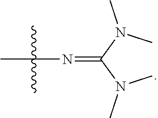 [SMILES: *N=C(N(C)C)N(C)C]

In some embodiments, one R′ on a nitrogen atom is taken with a R′ on the other nitrogen to form a ring as described herein.

In some embodiments, —X—R^{L} is

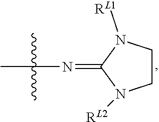

wherein R¹ and R² are independently R′. In some embodiments, —X—R^{L} is

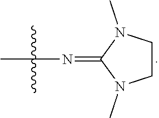 [SMILES: *N=C1N(C)CCN1C]

In some embodiments, —X—R^{L} is

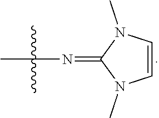 [SMILES: *N=c1n(C)ccn1C]

In some embodiments, two R′ on the same nitrogen are taken together to form a ring as described herein. In some embodiments, —X—R^{L} is

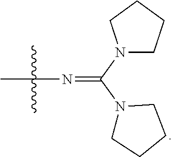 [SMILES: *N=C(N1CCCC1)N1CCCC1]

In some embodiments, —X—R^{L} is

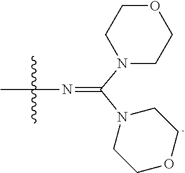 [SMILES: *N=C(N1CCOCC1)N1CCOCC1]

In some embodiments, —X—R^{L} is

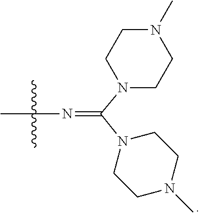 [SMILES: *N=C(N1CCN(C)CC1)N1CCN(C)CC1]

In some embodiments, —X—R^{L} is

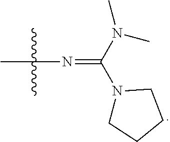 [SMILES: *N=C(N(C)C)N1CCCC1]

In some embodiments, —X—R^{L} is

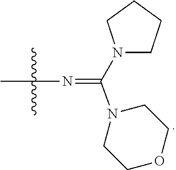 [SMILES: *N=C(N1CCCC1)N1CCOCC1]

In some embodiments, —X—R^{L} is

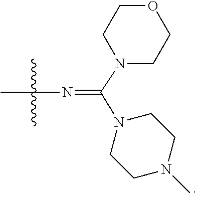 [SMILES: *N=C(N1CCOCC1)N1CCN(C)CC1]

In some embodiments, —X—R^{L} is

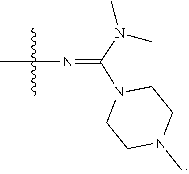 [SMILES: *N=C(N(C)C)N1CCN(C)CC1]

In some embodiments, —X—R^{L} is

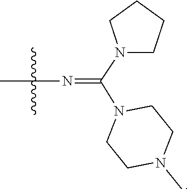 [SMILES: *N=C(N1CCCC1)N1CCN(C)CC1]

In some embodiments, —X—R^{L} is

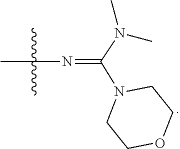 [SMILES: *N=C(N(C)C)N1CCOCC1]

In some embodiments, —X—R^{L} is R as described herein. In some embodiments, R is not hydrogen. In some embodiments, R is optionally substituted C₁₋₆ aliphatic. In some embodiments, R is optionally substituted C₁₋₆ alkyl. In some embodiments, R is methyl.

In some embodiments, —X—R^{L} is selected from Tables below. In some embodiments, X is as described herein. In some embodiments, R^{L} is as described herein. In some embodiments, a linkage has the structure of —Y—P^{L}(—X—R^{L})—Z—, wherein —X—R^{L} is selected from Tables below, and each other variable is independently as described herein. In some embodiments, a linkage has the structure of or comprises —P(O)(—X—R^{L})—, wherein —X—R^{L} is selected from Tables below. In some embodiments, a linkage has the structure of or comprises —P(S)(—X—R^{L})—, wherein —X—R^{L} is selected from Tables below. In some embodiments, a linkage has the structure of or comprises —P(—X—R^{L})—, wherein —X—R^{L} is selected from Tables below. In some embodiments, a linkage has the structure of or comprises —O—P(O)(—X—R^{L})—O—, wherein —X—R^{L} is selected from Tables below. In some embodiments, a linkage has the structure of or comprises —O—P(S)(—X—R^{L})—O—, wherein —X—R^{L} is selected from Tables below. In some embodiments, a linkage has the structure of or comprises —O—P(—X—R^{L})—O—, wherein —X—R^{L} is selected from Tables below. In some embodiments, a linkage has the structure of —O—P(O)(—X—R^{L})—O—, wherein —X—R^{L} is selected from Tables below. In some embodiments, a linkage has the structure of —O—P(S)(—X—R^{L})—O—, wherein —X—R^{L} is selected from Tables below. In some embodiments, a linkage has the structure of —O—P(—X—R^{L})—O—, wherein —X—R^{L} is selected from Tables below. In some embodiments, the Tables below, n is 0-20 or as described herein.

TABLE L-1
Certain useful moieties bonded to linkage phosphorus (e.g., —X—R^{L}).

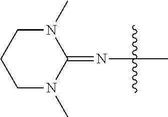

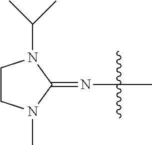

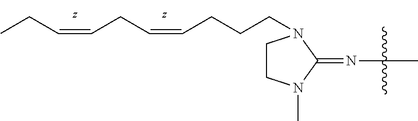

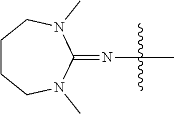

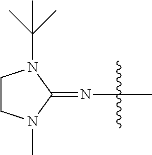

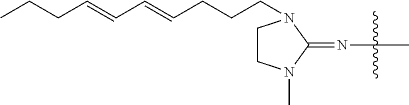

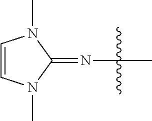

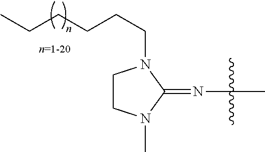

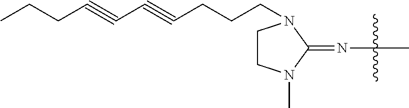

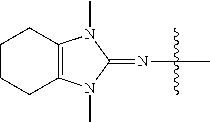

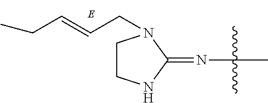

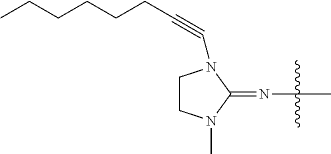

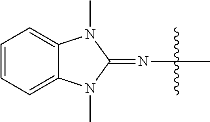

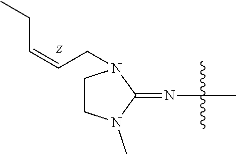

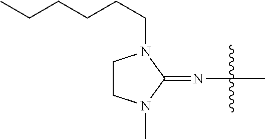

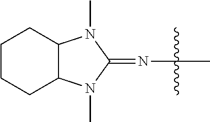

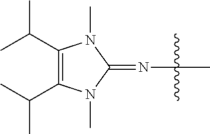

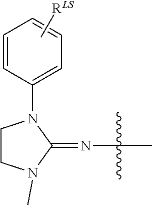

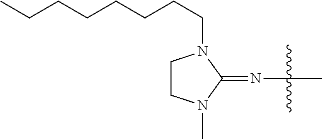

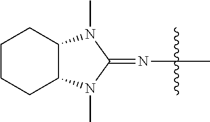

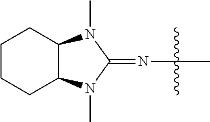

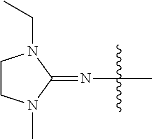

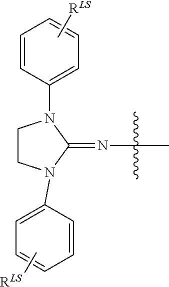

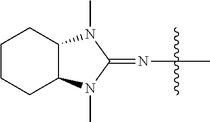

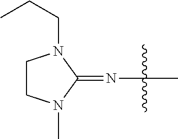

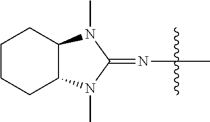

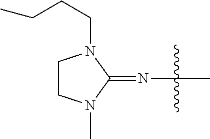

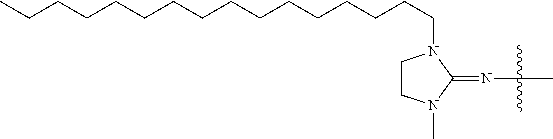

wherein each R^{LS} is independently R^{s}. In some embodiments, each R^{LS} is independently —Cl, —Br,
—F, —N(Me)₂, or —NHCOCH₃.

TABLE L-2
Certain useful moieties bonded to
linkage phosphorus (e.g., —X—R^{L}).

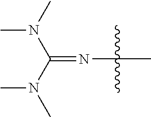 [SMILES: *N=C(N(C)C)N(C)C]

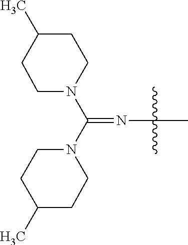 [SMILES: *N=C(N1CCC(C)CC1)N1CCC(C)CC1]

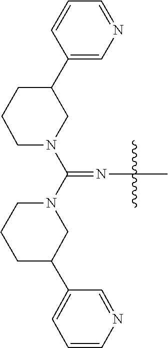 [SMILES: *N=C(N1CCCC(c2cccnc2)C1)N1CCCC(c2cccnc2)C1]

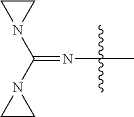 [SMILES: *N=C(N1CC1)N1CC1]

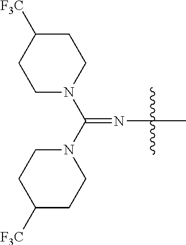 [SMILES: *N=C(N1CCC(C(F)(F)F)CC1)N1CCC(C(F)(F)F)CC1]

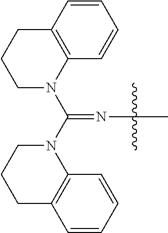 [SMILES: *N=C(N1CCCc2ccccc21)N1CCCc2ccccc21]

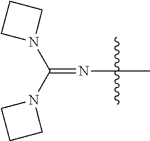 [SMILES: *N=C(N1CCC1)N1CCC1]

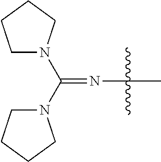 [SMILES: *N=C(N1CCCC1)N1CCCC1]

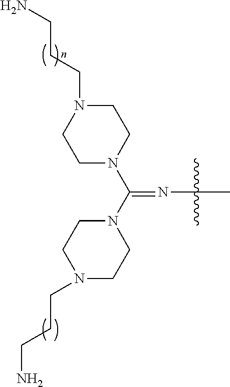

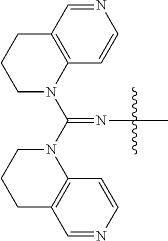 [SMILES: *N=C(N1CCCc2cnccc21)N1CCCc2cnccc21]

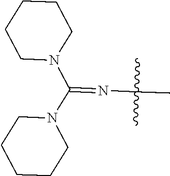 [SMILES: *N=C(N1CCCCC1)N1CCCCC1]

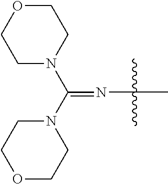 [SMILES: *N=C(N1CCOCC1)N1CCOCC1]

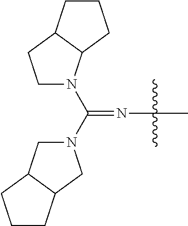 [SMILES: *N=C(N1CC2CCCC2C1)N1CCC2CCCC21]

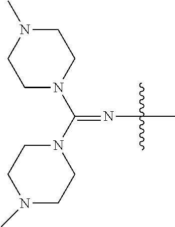 [SMILES: *N=C(N1CCN(C)CC1)N1CCN(C)CC1]

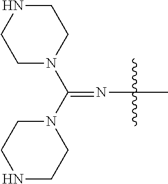 [SMILES: *N=C(N1CCNCC1)N1CCNCC1]

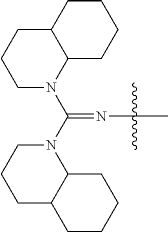 [SMILES: *N=C(N1CCCC2CCCCC21)N1CCCC2CCCCC21]

TABLE L-3
Certain useful moieties bonded to
linkage phosphorus (e.g., —X—R^{L}).

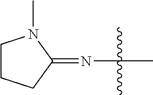 [SMILES: *N=C1CCCN1C]

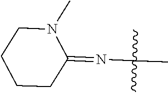 [SMILES: *N=C1CCCCN1C]

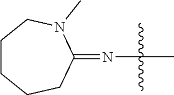 [SMILES: *N=C1CCCCCN1C]

TABLE L-4
Certain useful moieties bonded to linkage phosphorus (e.g., —X—R^{L}).

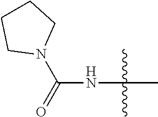 [SMILES: *NC(=O)N1CCCC1]

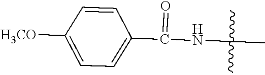 [SMILES: *NC(=O)c1ccc(OC)cc1]

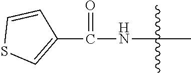 [SMILES: *NC(=O)c1ccsc1]

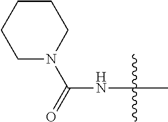 [SMILES: *NC(=O)N1CCCCC1]

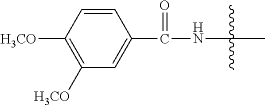 [SMILES: *NC(=O)c1ccc(OC)c(OC)c1]

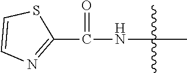 [SMILES: *NC(=O)c1nccs1]

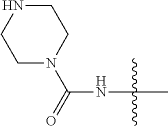 [SMILES: *NC(=O)N1CCNCC1]

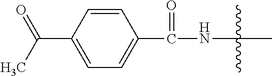 [SMILES: *NC(=O)c1ccc(C(C)=O)cc1]

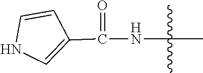 [SMILES: *NC(=O)c1cc[nH]c1]

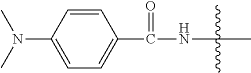 [SMILES: *NC(=O)c1ccc(N(C)C)cc1]

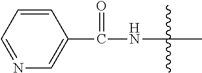 [SMILES: *NC(=O)c1cccnc1]

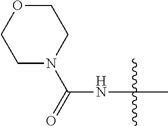 [SMILES: *NC(=O)N1CCOCC1]

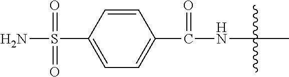 [SMILES: *NC(=O)c1ccc(S(N)(=O)=O)cc1]

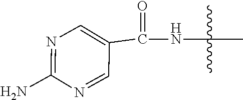 [SMILES: *NC(=O)c1cnc(N)nc1]

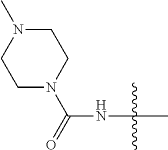 [SMILES: *NC(=O)N1CCN(C)CC1]

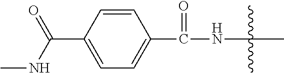 [SMILES: *NC(=O)c1ccc(C(=O)NC)cc1]

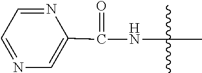 [SMILES: *NC(=O)c1cnccn1]

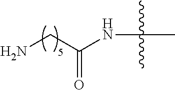

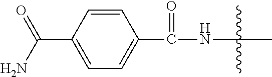 [SMILES: *NC(=O)c1ccc(C(N)=O)cc1]

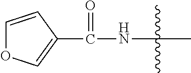 [SMILES: *NC(=O)c1ccoc1]

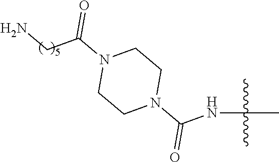 [SMILES: *NC(=O)N1CCN(C(=O)CN)CC1]

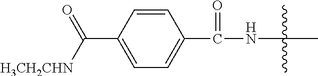

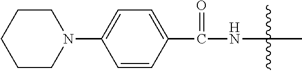 [SMILES: *NC(=O)c1ccc(N2CCCCC2)cc1]

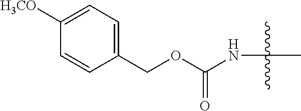 [SMILES: *NC(=O)OCc1ccc(OC)cc1]

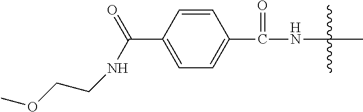 [SMILES: *NC(=O)c1ccc(C(=O)NCCOC)cc1]

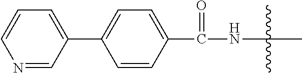 [SMILES: *NC(=O)c1ccc(-c2cccnc2)cc1]

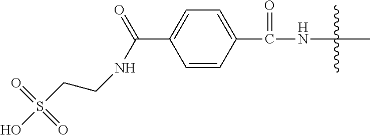 [SMILES: *NC(=O)c1ccc(C(=O)NCCS(=O)(=O)O)cc1]

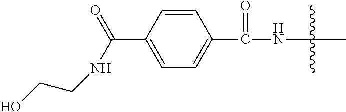 [SMILES: *NC(=O)c1ccc(C(=O)NCCO)cc1]

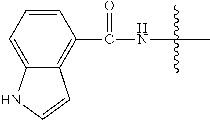 [SMILES: *NC(=O)c1cccc2[nH]ccc12]

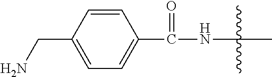 [SMILES: *NC(=O)c1ccc(CN)cc1]

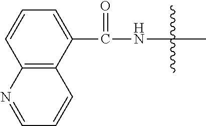 [SMILES: *NC(=O)c1cccc2ncccc12]

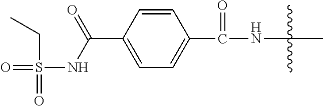 [SMILES: *NC(=O)c1ccc(C(=O)NS(=O)(=O)CC)cc1]

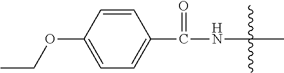 [SMILES: *NC(=O)c1ccc(OCC)cc1]

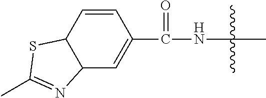 [SMILES: *NC(=O)C1=CC2N=C(C)SC2C=C1]

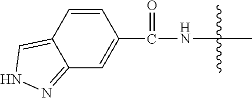 [SMILES: *NC(=O)c1ccc2c[nH]nc2c1]

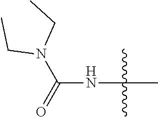 [SMILES: *NC(=O)N(CC)CC]

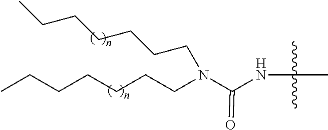 [SMILES: *NC(=O)N(CCCCCCC)CCCCCCC]

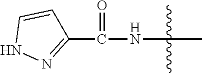 [SMILES: *NC(=O)c1cc[nH]n1]

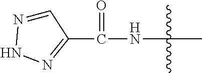 [SMILES: *NC(=O)c1cn[nH]n1]

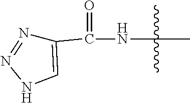 [SMILES: *NC(=O)c1c[nH]nn1]

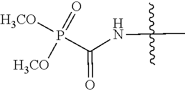 [SMILES: *NC(=O)P(=O)(OC)OC]

TABLE L-5
Certain useful moieties bonded to linkage phosphorus (e.g., —X—R^{L}).

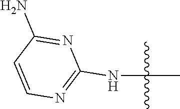 [SMILES: *Nc1nccc(N)n1]

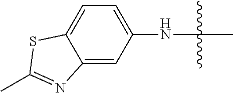 [SMILES: *Nc1ccc2sc(C)nc2c1]

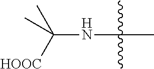 [SMILES: *NC(C)(C)C(=O)O]

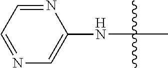 [SMILES: *Nc1cnccn1]

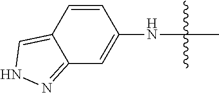 [SMILES: *Nc1ccc2c[nH]nc2c1]

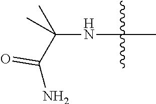 [SMILES: *NC(C)(C)C(N)=O]

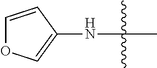 [SMILES: *Nc1ccoc1]

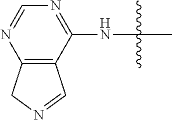 [SMILES: *Nc1ncnc2c1C=NC2]

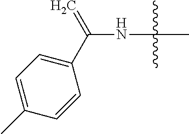 [SMILES: *NC(=C)c1ccc(C)cc1]

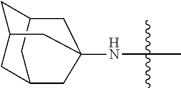 [SMILES: *NC12CC3CC(CC(C3)C1)C2]

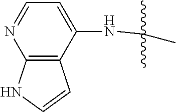 [SMILES: *Nc1ccnc2[nH]ccc12]

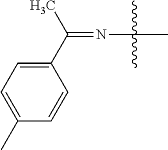 [SMILES: *N=C(C)c1ccc(C)cc1]

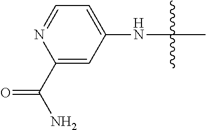 [SMILES: *Nc1ccnc(C(N)=O)c1]

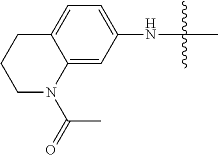 [SMILES: *Nc1ccc2c(c1)N(C(C)=O)CCC2]

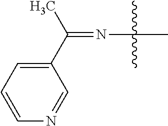 [SMILES: *N=C(C)c1cccnc1]

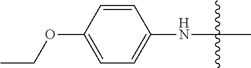 [SMILES: *Nc1ccc(OCC)cc1]

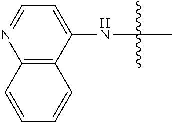 [SMILES: *Nc1ccnc2ccccc12]

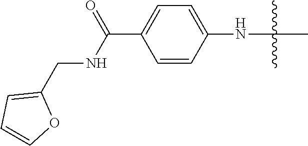 [SMILES: *Nc1ccc(C(=O)NCc2ccco2)cc1]

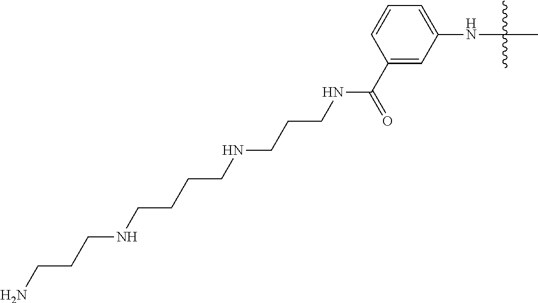 [SMILES: *Nc1cccc(C(=O)NCCCNCCCCNCCCN)c1]

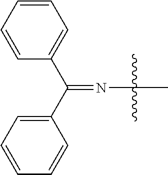 [SMILES: *N=C(c1ccccc1)c1ccccc1]

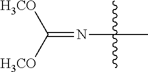 [SMILES: *N=C(OC)OC]

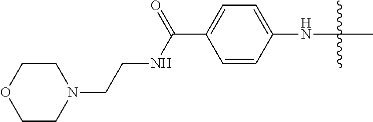 [SMILES: *Nc1ccc(C(=O)NCCN2CCOCC2)cc1]

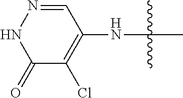 [SMILES: *Nc1cn[nH]c(=O)c1Cl]

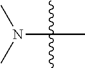 [SMILES: *N(C)C]

TABLE L-6
Certain useful moieties bonded to linkage phosphorus (e.g., —X—R^{L}).

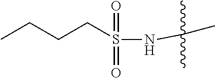 [SMILES: *NS(=O)(=O)CCCC]

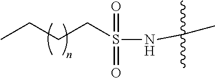

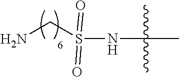

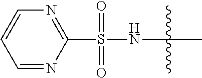 [SMILES: *NS(=O)(=O)c1ncccn1]

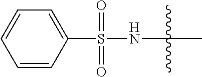 [SMILES: *NS(=O)(=O)c1ccccc1]

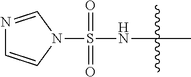 [SMILES: *NS(=O)(=O)n1ccnc1]

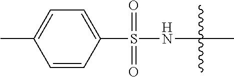 [SMILES: *NS(=O)(=O)c1ccc(C)cc1]

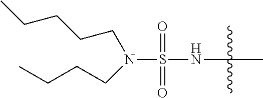 [SMILES: *NS(=O)(=O)N(CCCC)CCCCC]

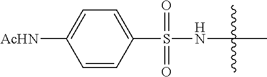 [SMILES: *NS(=O)(=O)c1ccc(NC(C)=O)cc1]

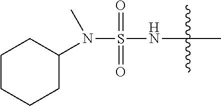 [SMILES: *NS(=O)(=O)N(C)C1CCCCC1]

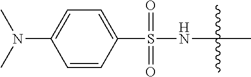 [SMILES: *NS(=O)(=O)c1ccc(N(C)C)cc1]

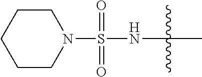 [SMILES: *NS(=O)(=O)N1CCCCC1]

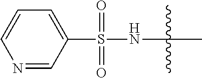 [SMILES: *NS(=O)(=O)c1cccnc1]

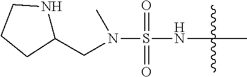 [SMILES: *NS(=O)(=O)N(C)CC1CCCN1]

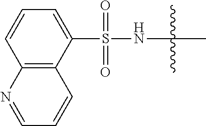 [SMILES: *NS(=O)(=O)c1cccc2ncccc12]

In some embodiments, an internucleotidic linkage, e.g., an non-negatively charged internucleotidic linkage or a neutral internucleotidic linkage, has the structure of -L^{L1}-_Cy¹L^{L2}-. In some embodiments, L^{L1} is bonded to a 3′-carbon of a sugar. In some embodiments, L^{L2} is bonded to a 5′-carbon of a sugar. In some embodiments, L^{L1} is —O—CH₂—. In some embodiments, L^{L2} is a covalent bond. In some embodiments, L^{L2} is a —N(R′)—. In some embodiments, L^{L2} is a —NH—. In some embodiments, L^{L2} is bonded to a 5′-carbon of a sugar, which 5′-carbon is substituted with ═O. In some embodiments, Cy^{IL} is optionally substituted 3-10 membered saturated, partially unsaturated, or aromatic ring having 0-5 heteroatoms. In some embodiments, Cy^{IL} is an optionally substituted triazole ring. In some embodiments, Cy^{IL} is

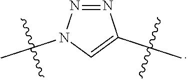 [SMILES: *c1cn(*)nn1]

In some embodiments, a linkage is

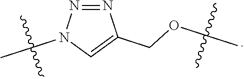 [SMILES: *OCc1cn(*)nn1]

In some embodiments, a non-negatively charged internucleotidic linkage has the structure of —OP(═W)(—N(R′)₂)—O—.

In some embodiments, R′ is R. In some embodiments, R′ is H. In some embodiments, R′ is —C(O)R. In some embodiments, R′ is —C(O)OR. In some embodiments, R′ is —S(O)₂R.

In some embodiments, R″ is —NHR′. In some embodiments, —N(R′)₂ is —NHR′.

As described herein, some embodiments, R is H. In some embodiments, R is optionally substituted C₁₋₆ aliphatic. In some embodiments, R is optionally substituted C₁₋₆ alkyl. In some embodiments, R is methyl. In some embodiments, R is substituted methyl. In some embodiments, R is ethyl. In some embodiments, R is substituted ethyl.

In some embodiments, as described herein, a non-negatively charged internucleotidic linkage is a neutral internucleotidic linkage.

In some embodiments, a modified internucleotidic linkage (e.g., a non-negatively charged internucleotidic linkage) comprises optionally substituted triazolyl. In some embodiments, R′ is or comprises optionally substituted triazolyl. In some embodiments, a modified internucleotidic linkage (e.g., a non-negatively charged internucleotidic linkage) comprises optionally substituted alkynyl. In some embodiments, R′ is optionally substituted alkynyl. In some embodiments, R′ comprises an optionally substituted triple bond. In some embodiments, a modified internucleotidic linkage comprises a triazole or alkyne moiety. In some embodiments, R′ is or comprises an optionally substituted triazole or alkyne moiety. In some embodiments, a triazole moiety, e.g., a triazolyl group, is optionally substituted. In some embodiments, a triazole moiety, e.g., a triazolyl group) is substituted. In some embodiments, a triazole moiety is unsubstituted. In some embodiments, a modified internucleotidic linkage comprises an optionally substituted guanidine moiety. In some embodiments, a modified internucleotidic linkage comprises an optionally substituted cyclic guanidine moiety. In some embodiments, R′, R^{L}, or —X—R^{L}, is or comprises an optionally substituted guanidine moiety. In some embodiments, R′, R^{L}, or —X—R^{L}, is or comprises an optionally substituted cyclic guanidine moiety. In some embodiments, R′, R^{L}, or —X—R^{L} comprises an optionally substituted cyclic guanidine moiety and an internucleotidic linkage has the structure of:

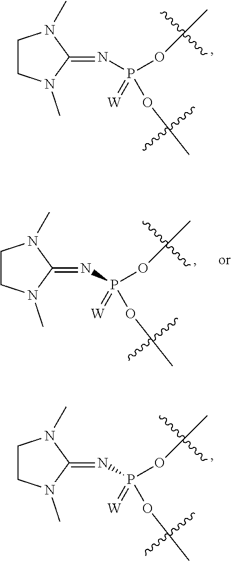

wherein W is O or S. In some embodiments, W is O. In some embodiments, W is S. In some embodiments, a non-negatively charged internucleotidic linkage is stereochemically controlled.

In some embodiments, a non-negatively charged internucleotidic linkage or a neutral internucleotidic linkage is an internucleotidic linkage comprising a triazole moiety. In some embodiments, a non-negatively charged internucleotidic linkage or a non-negatively charged internucleotidic linkage comprises an optionally substituted triazolyl group. In some embodiments, an internucleotidic linkage comprising a triazole moiety (e.g., an optionally substituted triazolyl group) has the structure of

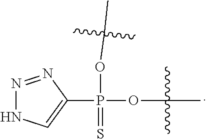 [SMILES: *OP(=S)(O*)c1c[nH]nn1]

In some embodiments, an internucleotidic linkage comprising a triazole moiety has the structure of

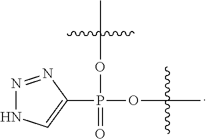 [SMILES: *OP(=O)(O*)c1c[nH]nn1]

In some embodiments, an internucleotidic linkage, e.g., a non-negatively charged internucleotidic linkage, a neutral internucleotidic linkage, comprises a cyclic guanidine moiety. In some embodiments, an internucleotidic linkage comprising a cyclic guanidine moiety has the structure of

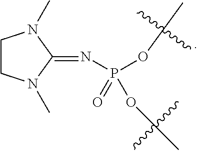 [SMILES: *OP(=O)(N=C1N(C)CCN1C)O*]

In some embodiments, anon-negatively charged internucleotidic linkage, or a neutral internucleotidic linkage, is or comprising a structure selected from

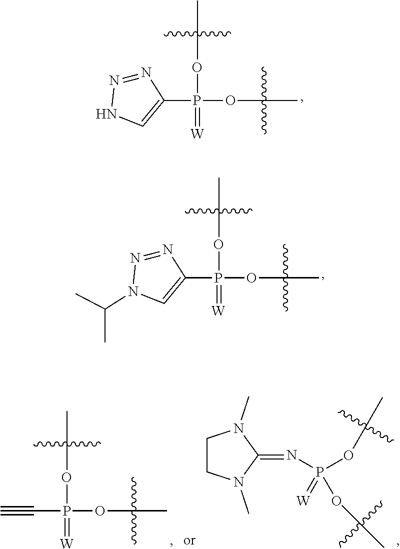

wherein W is O or S.

In some embodiments, an internucleotidic linkage comprises a Tmg group

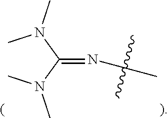 [SMILES: *N=C(N(C)C)N(C)C]

In some embodiments, an internucleotidic linkage comprises a Tmg group and has the structure of

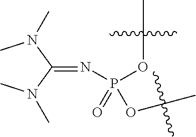 [SMILES: *OP(=O)(N=C(N(C)C)N(C)C)O*]

(the “Tmg internucleotidic linkage”). In some embodiments, neutral internucleotidic linkages include internucleotidic linkages of PNA and PMO, and an Tmg internucleotidic linkage.

In some embodiments, a non-negatively charged internucleotidic linkage comprises an optionally substituted 3-20 membered heterocyclyl or heteroaryl group having 1-10 heteroatoms. In some embodiments, a non-negatively charged internucleotidic linkage comprises an optionally substituted 3-20 membered heterocyclyl or heteroaryl group having 1-10 heteroatoms, wherein at least one heteroatom is nitrogen. In some embodiments, such a heterocyclyl or heteroaryl group is of a 5-membered ring. In some embodiments, such a heterocyclyl or heteroaryl group is of a 6-membered ring.

In some embodiments, a non-negatively charged internucleotidic linkage comprises an optionally substituted 5-20 membered heteroaryl group having 1-10 heteroatoms. In some embodiments, a non-negatively charged internucleotidic linkage comprises an optionally substituted 5-20 membered heteroaryl group having 1-10 heteroatoms, wherein at least one heteroatom is nitrogen. In some embodiments, a non-negatively charged internucleotidic linkage comprises an optionally substituted 5-6 membered heteroaryl group having 1-4 heteroatoms, wherein at least one heteroatom is nitrogen. In some embodiments, a non-negatively charged internucleotidic linkage comprises an optionally substituted 5-membered heteroaryl group having 1-4 heteroatoms, wherein at least one heteroatom is nitrogen. In some embodiments, a heteroaryl group is directly bonded to a linkage phosphorus. In some embodiments, a non-negatively charged internucleotidic linkage comprises an optionally substituted 5-20 membered heterocyclyl group having 1-10 heteroatoms. In some embodiments, a non-negatively charged internucleotidic linkage comprises an optionally substituted 5-20 membered heterocyclyl group having 1-10 heteroatoms, wherein at least one heteroatom is nitrogen. In some embodiments, a non-negatively charged internucleotidic linkage comprises an optionally substituted 5-6 membered heterocyclyl group having 1-4 heteroatoms, wherein at least one heteroatom is nitrogen. In some embodiments, a non-negatively charged internucleotidic linkage comprises an optionally substituted 5-membered heterocyclyl group having 1-4 heteroatoms, wherein at least one heteroatom is nitrogen. In some embodiments, at least two heteroatoms are nitrogen. In some embodiments, a heterocyclyl group is directly bonded to a linkage phosphorus. In some embodiments, a heterocyclyl group is bonded to a linkage phosphorus through a linker, e.g., ═N— when the heterocyclyl group is part of a guanidine moiety who directed bonded to a linkage phosphorus through its ═N—. In some embodiments, a non-negatively charged internucleotidic linkage comprises an optionally substituted

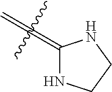 [SMILES: *=C1NCCN1]

group. In some embodiments, a non-negatively charged internucleotidic linkage comprises an substituted

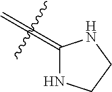 [SMILES: *=C1NCCN1]

group. In some embodiments, a non-negatively charged internucleotidic linkage comprises a

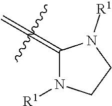

group. In some embodiments, each R¹ is independently optionally substituted C₁₋₆ alkyl. In some embodiments, each R¹ is independently methyl.

In some embodiments, a non-negatively charged internucleotidic linkage, e.g., a neutral internucleotidic linkage is not chirally controlled. In some embodiments, a non-negatively charged internucleotidic linkage is chirally controlled. In some embodiments, a non-negatively charged internucleotidic linkage is chirally controlled and its linkage phosphorus is Rp. In some embodiments, a non-negatively charged internucleotidic linkage is chirally controlled and its linkage phosphorus is Sp.

In some embodiments, an internucleotidic linkage comprises no linkage phosphorus. In some embodiments, an internucleotidic linkage has the structure of —C(O)—(O)— or —C(O)—N(R′)—, wherein R′ is as described herein. In some embodiments, an internucleotidic linkage has the structure of —C(O)—(O)—. In some embodiments, an internucleotidic linkage has the structure of —C(O)—N(R′)—, wherein R′ is as described herein. In various embodiments, —C(O)— is bonded to nitrogen. In some embodiments, an internucleotidic linkage is or comprises —C(O)—O— which is part of a carbamate moiety. In some embodiments, an internucleotidic linkage is or comprises —C(O)—O-which is part of a urea moiety.

In some embodiments, an oligonucleotide comprises 1-20, 1-15, 1-10, 1-5, or 1, 2, 3, 4, 5, 6, 7, 8, 9, 10, or more non-negatively charged internucleotidic linkages. In some embodiments, an oligonucleotide comprises 1-20, 1-15, 1-10, 1-5, or 1, 2, 3, 4, 5, 6, 7, 8, 9, 10, or more neutral internucleotidic linkages. In some embodiments, each of non-negatively charged internucleotidic linkage and/or neutral internucleotidic linkages is optionally and independently chirally controlled. In some embodiments, each non-negatively charged internucleotidic linkage in an oligonucleotide is independently a chirally controlled internucleotidic linkage. In some embodiments, each neutral internucleotidic linkage in an oligonucleotide is independently a chirally controlled internucleotidic linkage. In some embodiments, at least one non-negatively charged internucleotidic linkage/neutral internucleotidic linkage has the structure of

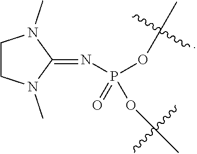 [SMILES: *OP(=O)(N=C1N(C)CCN1C)O*]

In some embodiments, an oligonucleotide comprises at least one non-negatively charged internucleotidic linkage wherein its linkage phosphorus is in Rp configuration, and at least one non-negatively charged internucleotidic linkage wherein its linkage phosphorus is in Sp configuration.

In many embodiments, as demonstrated extensively, oligonucleotides of the present disclosure comprise two or more different internucleotidic linkages. In some embodiments, an oligonucleotide comprises a phosphorothioate internucleotidic linkage and a non-negatively charged internucleotidic linkage. In some embodiments, an oligonucleotide comprises a phosphorothioate internucleotidic linkage, a non-negatively charged internucleotidic linkage, and a natural phosphate linkage. In some embodiments, a non-negatively charged internucleotidic linkage is a neutral internucleotidic linkage. In some embodiments, a non-negatively charged internucleotidic linkage is n001, n003, n004, n006, n008 or n009, n013, n020, n021, n025, n026, n029, n031, n037, n046, n047, n048, n054, or n055). In some embodiments, a non-negatively charged internucleotidic linkage is n001. In some embodiments, each phosphorothioate internucleotidic linkage is independently chirally controlled. In some embodiments, each chiral modified internucleotidic linkage is independently chirally controlled. In some embodiments, one or more non-negatively charged internucleotidic linkage are not chirally controlled.

A typical connection, as in natural DNA and RNA, is that an internucleotidic linkage forms bonds with two sugars (which can be either unmodified or modified as described herein). In many embodiments, as exemplified herein an internucleotidic linkage forms bonds through its oxygen atoms or heteroatoms with one optionally modified ribose or deoxyribose at its 5′ carbon, and the other optionally modified ribose or deoxyribose at its 3′ carbon. In some embodiments, internucleotidic linkages connect sugars that are not ribose sugars, e.g., sugars comprising N ring atoms and acyclic sugars as described herein.

In some embodiments, each nucleoside units connected by an internucleotidic linkage independently comprises a nucleobase which is independently an optionally substituted A, T, C, G, or U, or an optionally substituted tautomer of A, T, C, G or U.

In some embodiments, an oligonucleotide comprises a modified internucleotidic linkage (e.g., a modified internucleotidic linkage having the structure of Formula I, I-a, I-b, or I-c, I-n-1, I-n-2, I-n-3, I-n-4, II, II-a-1, II-a-2, II-b-1, II-b-2, II-c-1, II-c-2, II-d-1, II-d-2, etc., or a salt form thereof) as described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612 the internucleotidic linkages (e.g., those of Formula I, I-a, I-b, or I-c, I-n-1, I-n-2, I-n-3, I-n-4, II, II-a-1, II-a-2, II-b-1, II-b-2, II-c-1, II-c-2, II-d-1, II-d-2, etc.,) of each of which are independently incorporated herein by reference. In some embodiments, a modified internucleotidic linkage is a non-negatively charged internucleotidic linkage. In some embodiments, provided oligonucleotides comprise one or more non-negatively charged internucleotidic linkages. In some embodiments, a non-negatively charged internucleotidic linkage is a positively charged internucleotidic linkage. In some embodiments, a non-negatively charged internucleotidic linkage is a neutral internucleotidic linkage. In some embodiments, the present disclosure provides oligonucleotides comprising one or more neutral internucleotidic linkages. In some embodiments, a non-negatively charged internucleotidic linkage or a neutral internucleotidic linkage (e.g., one of Formula I-n-1, I-n-2, I-n-3, I-n-4, II, II-a-1, II-a-2, II-b-1, II-b-2, II-c-1, II-c-2, II-d-1, II-d-2, etc.) is as described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612. In some embodiments, a non-negatively charged internucleotidic linkage or neutral internucleotidic linkage is one of Formula I-n-1, I-n-2, I-n-3, I-n-4, II, II-a-1, II-a-2, II-b-1, II-b-2, II-c-1, II-c-2, II-d-1, II-d-2, etc. as described in WO 2018/223056, WO 2019/032607, WO 2019/075357, WO 2019/032607, WO 2019/075357, WO 2019/200185, WO 2019/217784, and/or WO 2019/032612, such internucleotidic linkages of each of which are independently incorporated herein by reference.

As described herein, various variables can be R, e.g., R′, R^{L}, etc. Various embodiments for R are described in the present disclosure (e.g., when describing variables that can be R). Such embodiments are generally useful for all variables that can be R. In some embodiments, R is hydrogen. In some embodiments, R is optionally substituted C₁₋₃₀ (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30) aliphatic. In some embodiments, R is optionally substituted C₁₋₂₀ aliphatic. In some embodiments, R is optionally substituted C₁₋₁₀ aliphatic. In some embodiments, R is optionally substituted C₁₋₆ aliphatic. In some embodiments, R is optionally substituted alkyl. In some embodiments, R is optionally substituted C₁₋₆ alkyl. In some embodiments, R is optionally substituted methyl. In some embodiments, R is methyl. In some embodiments, R is optionally substituted ethyl. In some embodiments, R is optionally substituted propyl. In some embodiments, R is isopropyl. In some embodiments, R is optionally substituted butyl. In some embodiments, R is optionally substituted pentyl. In some embodiments, R is optionally substituted hexyl.

In some embodiments, R is optionally substituted 3-30 membered (e.g., 3, 4, 5, 6, 7, 8, 9, 10, 11, 12, 13, 14, 15, 16, 17, 18, 19, 20, 21, 22, 23, 24, 25, 26, 27, 28, 29 or 30) cycloaliphatic. In some embodiments, R is optionally substituted cycloalkyl. In some embodiments, cycloaliphatic is monocyclic, bicyclic, or polycyclic, wherein each monocyclic unit is independently saturated or partially saturated. In some embodiments, R is optionally substituted cyclopropyl. In some embodiments, R is optionally substituted cyclobutyl. In some embodiments, R is optionally substituted cyclopentyl. In some embodiments, R is optionally substituted cyclohexyl. In some embodiments, R is optionally substituted adamantyl.

In some embodiments, R is optionally substituted C₁₋₃₀ (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30) heteroaliphatic having 1-10 heteroatoms. In some embodiments, R is optionally substituted C₁₋₂₀ aliphatic having 1-10 heteroatoms. In some embodiments, R is optionally substituted C₁₋₁₀ aliphatic having 1-10 heteroatoms. In some embodiments, R is optionally substituted C₁₋₆ aliphatic having 1-3 heteroatoms. In some embodiments, R is optionally substituted heteroalkyl. In some embodiments, R is optionally substituted C₁₋₆ heteroalkyl. In some embodiments, R is optionally substituted 3-30 membered (e.g., 3, 4, 5, 6, 7, 8, 9, 10, 11, 12, 13, 14, 15, 16, 17, 18, 19, 20, 21, 22, 23, 24, 25, 26, 27, 28, 29 or 30) heterocycloaliphatic having 1-10 heteroatoms. In some embodiments, R is optionally substituted heteroclycloalkyl. In some embodiments, heterocycloaliphatic is monocyclic, bicyclic, or polycyclic, wherein each monocyclic unit is independently saturated or partially saturated.

In some embodiments, R is optionally substituted C₆₋₃₀ aryl. In some embodiments, R is optionally substituted phenyl. In some embodiments, R is optionally substituted phenyl. In some embodiments, R is C₆₋₁₄ aryl. In some embodiments, R is optionally substituted bicyclic aryl. In some embodiments, R is optionally substituted polycyclic aryl. In some embodiments, R is optionally substituted C₆₋₃₀ arylaliphatic. In some embodiments, R is C₆₋₃₀ arylheteroaliphatic having 1-10 heteroatoms.

In some embodiments, R is optionally substituted 5-30 (5, 6, 7, 8, 9, 10, 11, 12, 13, 14, 15, 16, 17, 18, 19, 20, 21, 22, 23, 24, 25, 26, 27, 28, 29 or 30) membered heteroaryl having 1-10 heteroatoms. In some embodiments, R is optionally substituted 5-20 membered heteroaryl having 1-10 heteroatoms. In some embodiments, R is optionally substituted 5-10 membered heteroaryl having 1-10 heteroatoms. In some embodiments, R is optionally substituted 5-membered heteroaryl having 1-5 heteroatoms. In some embodiments, R is optionally substituted 5-membered heteroaryl having 1-4 heteroatoms. In some embodiments, R is optionally substituted 5-membered heteroaryl having 1-3 heteroatoms. In some embodiments, R is optionally substituted 5-membered heteroaryl having 1-2 heteroatoms. In some embodiments, R is optionally substituted 5-membered heteroaryl having one heteroatom. In some embodiments, R is optionally substituted 6-membered heteroaryl having 1-5 heteroatoms. In some embodiments, R is optionally substituted 6-membered heteroaryl having 1-4 heteroatoms. In some embodiments, R is optionally substituted 6-membered heteroaryl having 1-3 heteroatoms. In some embodiments, R is optionally substituted 6-membered heteroaryl having 1-2 heteroatoms. In some embodiments, R is optionally substituted 6-membered heteroaryl having one heteroatom. In some embodiments, R is optionally substituted monocyclic heteroaryl. In some embodiments, R is optionally substituted bicyclic heteroaryl. In some embodiments, R is optionally substituted polycyclic heteroaryl. In some embodiments, a heteroatom is nitrogen.

In some embodiments, R is optionally substituted 2-pyridinyl. In some embodiments, R is optionally substituted 3-pyridinyl. In some embodiments, R is optionally substituted 4-pyridinyl. In some embodiments, R is optionally substituted

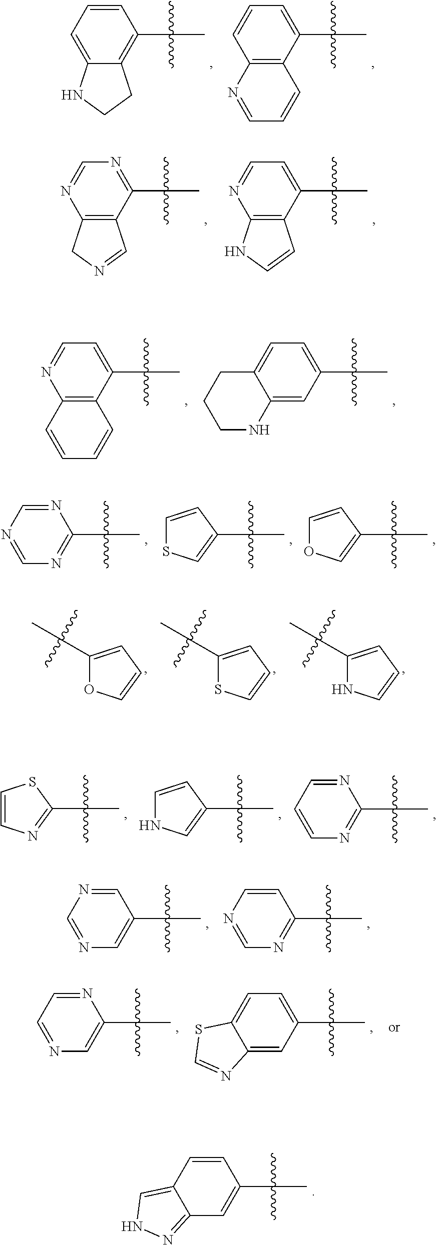

In some embodiments, R is optionally substituted 3-30 (3, 4, 5, 6, 7, 8, 9, 10, 11, 12, 13, 14, 15, 16, 17, 18, 19, 20, 21, 22, 23, 24, 25, 26, 27, 28, 29 or 30) membered heterocyclyl having 1-10 heteroatoms. In some embodiments, R is optionally substituted 3-membered heterocyclyl having 1-2 heteroatoms. In some embodiments, R is optionally substituted 4-membered heterocyclyl having 1-2 heteroatoms. In some embodiments, R is optionally substituted 5-20 membered heterocyclyl having 1-10 heteroatoms. In some embodiments, R is optionally substituted 5-10 membered heterocyclyl having 1-10 heteroatoms. In some embodiments, R is optionally substituted 5-membered heterocyclyl having 1-5 heteroatoms. In some embodiments, R is optionally substituted 5-membered heterocyclyl having 1-4 heteroatoms. In some embodiments, R is optionally substituted 5-membered heterocyclyl having 1-3 heteroatoms. In some embodiments, R is optionally substituted 5-membered heterocyclyl having 1-2 heteroatoms. In some embodiments, R is optionally substituted 5-membered heterocyclyl having one heteroatom. In some embodiments, R is optionally substituted 6-membered heterocyclyl having 1-5 heteroatoms. In some embodiments, R is optionally substituted 6-membered heterocyclyl having 1-4 heteroatoms. In some embodiments, R is optionally substituted 6-membered heterocyclyl having 1-3 heteroatoms. In some embodiments, R is optionally substituted 6-membered heterocyclyl having 1-2 heteroatoms. In some embodiments, R is optionally substituted 6-membered heterocyclyl having one heteroatom. In some embodiments, R is optionally substituted monocyclic heterocyclyl. In some embodiments, R is optionally substituted bicyclic heterocyclyl. In some embodiments, R is optionally substituted polycyclic heterocyclyl. In some embodiments, R is optionally substituted saturated heterocyclyl. In some embodiments, R is optionally substituted partially unsaturated heterocyclyl. In some embodiments, a heteroatom is nitrogen. In some embodiments, R is optionally substituted

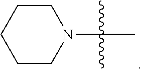 [SMILES: *N1CCCCC1]

In some embodiments, R is optionally substituted

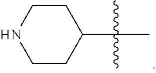 [SMILES: *C1CCNCC1]

In some embodiments, R is optionally substituted

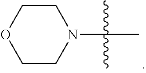 [SMILES: *N1CCOCC1]

In some embodiments, two R groups are optionally and independently taken together to form a covalent bond. In some embodiments, two or more R groups on the same atom are optionally and independently taken together with the atom to form an optionally substituted, 3-30 membered, monocyclic, bicyclic or polycyclic ring having, in addition to the atom, 0-10 heteroatoms. In some embodiments, two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

Various variables may comprise an optionally substituted ring, or can be taken together with their intervening atom(s) to form a ring. In some embodiments, a ring is 3-30 (e.g., 3, 4, 5, 6, 7, 8, 9, 10, 11, 12, 13, 14, 15, 16, 17, 18, 19, 20, 21, 22, 23, 24, 25, 26, 27, 28, 29 or 30) membered. In some embodiments, a ring is 3-20 membered. In some embodiments, a ring is 3-15 membered. In some embodiments, a ring is 3-10 membered. In some embodiments, a ring is 3-8 membered. In some embodiments, a ring is 3-7 membered. In some embodiments, a ring is 3-6 membered. In some embodiments, a ring is 4-20 membered. In some embodiments, a ring is 5-20 membered. In some embodiments, a ring is monocyclic. In some embodiments, a ring is bicyclic. In some embodiments, a ring is polycyclic. In some embodiments, each monocyclic ring or each monocyclic ring unit in bicyclic or polycyclic rings is independently saturated, partially saturated or aromatic. In some embodiments, each monocyclic ring or each monocyclic ring unit in bicyclic or polycyclic rings is independently 3-10 membered and has 0-5 heteroatoms.

In some embodiments, each heteroatom is independently selected oxygen, nitrogen, sulfur, silicon, and phosphorus. In some embodiments, each heteroatom is independently selected oxygen, nitrogen, sulfur, and phosphorus. In some embodiments, each heteroatom is independently selected oxygen, nitrogen, and sulfur. In some embodiments, a heteroatom is in an oxidized form.

As appreciated by those skilled in the art, many other types of internucleotidic linkages may be utilized in accordance with the present disclosure, for example, those described in U.S. Pat. Nos. 3,687,808; 4,469,863; 4,476,301; 5,177,195; 5,023,243; 5,034,506; 5,166,315; 5,185,444; 5,188,897; 5,214,134; 5,216,141; 5,235,033; 5,264,423; 5,264,564; 5,276,019; 5,278,302; 5,286,717; 5,321,131; 5,399,676; 5,405,938; 5,405,939; 5,434,257; 5,453,496; 5,455,233; 5,466,677; 5,466,677; 5,470,967; 5,476,925; 5,489,677; 5,519,126; 5,536,821; 5,541,307; 5,541,316; 5,550,111; 5,561,225; 5,563,253; 5,571,799; 5,587,361; 5,596,086; 5,602,240; 5,608,046; 5,610,289; 5,618,704; 5,623,070; 5,625,050; 5,633,360; 5,64,562; 5,663,312; 5,677,437; 5,677,439; 6,160,109; 6,239,265; 6,028,188; 6,124,445; 6,169,170; 6,172,209; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; or RE39464. In certain embodiments, a modified internucleotidic linkage is one described in U.S. Pat. No. 9,982,257, US 20170037399, US 20180216108, WO 2017192664, WO 2017015575, WO2017062862, WO 2018067973, WO 2017160741, WO 2017192679, WO 2017210647, WO 2018098264, PCT/US18/35687, PCT/US18/38835, or PCT/US18/51398, the nucleobases, sugars, internucleotidic linkages, chiral auxiliaries/reagents, and technologies for oligonucleotide synthesis (reagents, conditions, cycles, etc.) of each of which is independently incorporated herein by reference.

In certain embodiments, each internucleotidic linkage in a ds oligonucleotide is independently selected from a natural phosphate linkage, a phosphorothioate linkage, and a non-negatively charged internucleotidic linkage (e.g., n001). In certain embodiments, each internucleotidic linkage in a ds oligonucleotide is independently selected from a natural phosphate linkage, a phosphorothioate linkage, and a neutral internucleotidic linkage (e.g., n001).

In certain embodiments, a ds oligonucleotide comprises one or more nucleotides that independently comprise a phosphorus modification prone to “autorelease” under certain conditions. That is, under certain conditions, a particular phosphorus modification is designed such that it self-cleaves from the ds oligonucleotide to provide, e.g., a natural phosphate linkage. In certain embodiments, such a phosphorus modification has a structure of —O-L-R¹, wherein L is L^{B} as described herein, and R¹ is R′ as described herein. In certain embodiments, a phosphorus modification has a structure of —S-L-R¹, wherein each L and R¹ is independently as described in the present disclosure. Certain examples of such phosphorus modification groups can be found in U.S. Pat. No. 9,982,257. In certain embodiments, an autorelease group comprises a morpholino group. In certain embodiments, an autorelease group is characterized by the ability to deliver an agent to the internucleotidic phosphorus linker, which agent facilitates further modification of the phosphorus atom such as, e.g., desulfurization. In certain embodiments, the agent is water and the further modification is hydrolysis to form a natural phosphate linkage.

In certain embodiments, a ds oligonucleotide comprises one or more internucleotidic linkages that improve one or more pharmaceutical properties and/or activities of the oligonucleotide. It is well documented in the art that certain oligonucleotides are rapidly degraded by nucleases and exhibit poor cellular uptake through the cytoplasmic cell membrane (Poijarvi-Virta et al., Curr. Med. Chem. (2006), 13(28); 3441-65; Wagner et al., Med. Res. Rev. (2000), 20(6):417-51; Peyrottes et al., Mini Rev. Med. Chem. (2004), 4(4):395-408; Gosselin et al., (1996), 43(1):196-208; Bologna et al., (2002), Antisense & Nucleic Acid Drug Development 12:33-41). Vives et al. (Nucleic Acids Research (1999), 27(20):4071-76) reported that tert-butyl SATE pro-oligonucleotides displayed markedly increased cellular penetration compared to the parent oligonucleotide under certain conditions.

Ds oligonucleotides can comprise various number of natural phosphate linkages. In certain embodiments, 5% or more of the internucleotidic linkages of provided ds oligonucleotides are natural phosphate linkages. In certain embodiments, 10% or more of the internucleotidic linkages of provided ds oligonucleotides are natural phosphate linkages. In certain embodiments, 15% or more of the internucleotidic linkages of provided ds oligonucleotides are natural phosphate linkages. In certain embodiments, 20% or more of the internucleotidic linkages of provided ds oligonucleotides are natural phosphate linkages. In certain embodiments, 25% or more of the internucleotidic linkages of provided ds oligonucleotides are natural phosphate linkages. In certain embodiments, 30% or more of the internucleotidic linkages of provided ds oligonucleotides are natural phosphate linkages. In certain embodiments, 35% or more of the internucleotidic linkages of provided ds oligonucleotides are natural phosphate linkages. In certain embodiments, 40% or more of the internucleotidic linkages of provided ds oligonucleotides are natural phosphate linkages. In certain embodiments, provided ds oligonucleotides comprises 1, 2, 3, 4, 5, 6, 7, 8, 9, 10 or more natural phosphate linkages. In certain embodiments, provided ds oligonucleotides comprises 4, 5, 6, 7, 8, 9, 10 or more natural phosphate linkages. In certain embodiments, the number of natural phosphate linkages is 2. In certain embodiments, the number of natural phosphate linkages is 3. In certain embodiments, the number of natural phosphate linkages is 4. In certain embodiments, the number of natural phosphate linkages is 5. In certain embodiments, the number of natural phosphate linkages is 6. In certain embodiments, the number of natural phosphate linkages is 7. In certain embodiments, the number of natural phosphate linkages is 8. In certain embodiments, some or all of the natural phosphate linkages are consecutive.

In certain embodiments, the present disclosure demonstrates that, in at least some cases, Sp internucleotidic linkages, among other things, at the 5′- and/or 3′-end can improve ds oligonucleotide stability. In certain embodiments, the present disclosure demonstrates that, among other things, natural phosphate linkages and/or Rp internucleotidic linkages may improve removal of ds oligonucleotides from a system. As appreciated by a person having ordinary skill in the art, various assays known in the art can be utilized to assess such properties in accordance with the present disclosure.

In certain embodiments, each phosphorothioate internucleotidic linkage in a ds oligonucleotide or a portion thereof (e.g., a domain, a subdomain, etc.) is independently chirally controlled. In certain embodiments, each is independently Sp or Rp. In certain embodiments, a high level is Sp as described herein. In certain embodiments, each phosphorothioate internucleotidic linkage in a ds oligonucleotide or a portion thereof is chirally controlled and is Sp. In certain embodiments, one or more, e.g., about 1-5 (e.g., about 1, 2, 3, 4, or 5) is Rp.

In certain embodiments, as illustrated in certain examples, a ds oligonucleotide or a portion thereof comprises one or more non-negatively charged internucleotidic linkages, each of which is optionally and independently chirally controlled. In certain embodiments, each non-negatively charged internucleotidic linkage is independently n001. In certain embodiments, a chiral non-negatively charged internucleotidic linkage is not chirally controlled. In certain embodiments, each chiral non-negatively charged internucleotidic linkage is not chirally controlled. In certain embodiments, a chiral non-negatively charged internucleotidic linkage is chirally controlled. In certain embodiments, a chiral non-negatively charged internucleotidic linkage is chirally controlled and is Rp. In certain embodiments, a chiral non-negatively charged internucleotidic linkage is chirally controlled and is Sp. In certain embodiments, each chiral non-negatively charged internucleotidic linkage is chirally controlled. In certain embodiments, the number of non-negatively charged internucleotidic linkages in a ds oligonucleotide or a portion thereof is about 1-10, or about 1, 2, 3, 4, 5, 6, 7, 8, 9, or 10. In certain embodiments, it is about 1. In certain embodiments, it is about 2. In certain embodiments, it is about 3. In certain embodiments, it is about 4. In certain embodiments, it is about 5. In certain embodiments, it is about 6. In certain embodiments, it is about 7. In certain embodiments, it is about 8. In certain embodiments, it is about 9. In certain embodiments, it is about 10. In certain embodiments, two or more non-negatively charged internucleotidic linkages are consecutive. In certain embodiments, no two non-negatively charged internucleotidic linkages are consecutive. In certain embodiments, all non-negatively charged internucleotidic linkages in a ds oligonucleotide or a portion thereof are consecutive (e.g., 3 consecutive non-negatively charged internucleotidic linkages). In certain embodiments, a non-negatively charged internucleotidic linkage, or two or more (e.g., about 2, about 3, about 4 etc.) consecutive non-negatively charged internucleotidic linkages, are at the 3′-end of a ds oligonucleotide or a portion thereof. In certain embodiments, the last two or three or four internucleotidic linkages of a ds oligonucleotide or a portion thereof comprise at least one internucleotidic linkage that is not a non-negatively charged internucleotidic linkage. In certain embodiments, the last two or three or four internucleotidic linkages of a ds oligonucleotide or a portion thereof comprise at least one internucleotidic linkage that is not n001. In certain embodiments, the internucleotidic linkage linking the first two nucleosides of a ds oligonucleotide or a portion thereof is a non-negatively charged internucleotidic linkage. In certain embodiments, the internucleotidic linkage linking the last two nucleosides of a ds oligonucleotide or a portion thereof is a non-negatively charged internucleotidic linkage. In certain embodiments, the internucleotidic linkage linking the first two nucleosides of a ds oligonucleotide or a portion thereof is a phosphorothioate internucleotidic linkage. In certain embodiments, it is Sp. In certain embodiments, the internucleotidic linkage linking the last two nucleosides of a ds oligonucleotide or a portion thereof is a phosphorothioate internucleotidic linkage. In certain embodiments, it is Sp.

In certain embodiments, one or more chiral internucleotidic linkages are chirally controlled and one or more chiral internucleotidic linkages are not chirally controlled. In certain embodiments, each phosphorothioate internucleotidic linkage is independently chirally controlled, and one or more non-negatively charged internucleotidic linkage are not chirally controlled. In certain embodiments, each phosphorothioate internucleotidic linkage is independently chirally controlled, and each non-negatively charged internucleotidic linkage is not chirally controlled. In certain embodiments, the internucleotidic linkage between the first two nucleosides of a ds oligonucleotide is a non-negatively charged internucleotidic linkage. In certain embodiments, the internucleotidic linkage between the last two nucleosides are each independently a non-negatively charged internucleotidic linkage. In certain embodiments, both are independently non-negatively charged internucleotidic linkages. In certain embodiments, each non-negatively charged internucleotidic linkage is independently neutral internucleotidic linkage. In certain embodiments, each non-negatively charged internucleotidic linkage is independently n001.

In certain embodiments, a controlled level of ds oligonucleotides in a composition are desired ds oligonucleotides. In certain embodiments, of all ds oligonucleotides in a composition that share a common base sequence (e.g., a desired sequence for a purpose), or of all ds oligonucleotides in a composition, level of desired ds oligonucleotides (which may exist in various forms (e.g., salt forms) and typically differ only at non-chirally controlled internucleotidic linkages (various forms of the same stereoisomer can be considered the same for this purpose)) is about 5%-100%, 10%-100%, 20%-100%, 30%-100%, 40%-100%, 50%-100%, 60%-100%, 70%-100%, 80-100%, 90-100%, 95-100%, 50%-90%, about 5%, 10%, 15%, 20%, 25%, 30%, 35%, 40%, 45%, 50%, 55%, 60%, 65%, 70%, 75%, 80%, 85%, 90%, 95%, or 100%, or at least 5%, 10%, 15%, 20%, 25%, 30%, 35%, 40%, 45%, 50%, 55%, 60%, 65%, 70%, 75%, 80%, 85%, 90%, 95%, or 99%. In certain embodiments, a level is at least about 50%. In certain embodiments, a level is at least about 60%. In certain embodiments, a level is at least about 70%. In certain embodiments, a level is at least about 75%. In certain embodiments, a level is at least about 80%. In certain embodiments, a level is at least about 85%. In certain embodiments, a level is at least about 90%. In certain embodiments, a level is or is at least (DS)^{nc}, wherein DS is about 90%, 91%, 92%, 93%, 94%, 95%, 96%, 97%, 98%, 99% or 99.5% and nc is the number of chirally controlled internucleotidic linkages as described in the present disclosure (e.g., 1-50, 1-40, 1-30, 1-25, 1-20, 5-50, 5-40, 5-30, 5-25, 5-20, 1, 2, 3, 4, 5, 6, 7, 8, 9, 10, 11, 12, 13, 14, 15, 16, 17, 18, 19, 20, 21, 22, 23, 24, 25 or more). In certain embodiments, a level is or is at least (DS)^{nc}, wherein DS is 95%-100%.

Various types of internucleotidic linkages may be utilized in combination of other structural elements, e.g., sugars, to achieve desired ds oligonucleotide properties and/or activities. For example, the present disclosure routinely utilizes modified internucleotidic linkages and modified sugars, optionally with natural phosphate linkages and natural sugars, in designing ds oligonucleotides. In certain embodiments, the present disclosure provides a ds oligonucleotide comprising one or more modified sugars. In certain embodiments, the present disclosure provides a ds oligonucleotide comprising one or more modified sugars and one or more modified internucleotidic linkages, one or more of which are natural phosphate linkages.

### 2.3. Double Stranded Oligonucleotide Compositions

Among other things, the present disclosure provides various ds oligonucleotide compositions. In certain embodiments, the present disclosure provides ds oligonucleotide compositions of ds oligonucleotides described herein. In certain embodiments, a ds oligonucleotide composition, e.g., a dsRNAi oligonucleotide composition, comprises a plurality of a ds oligonucleotide described in the present disclosure. In certain embodiments, a ds oligonucleotide composition, e.g., a dsRNAi oligonucleotide composition, is chirally controlled. In certain embodiments, a ds oligonucleotide composition, e.g., a dsRNAi oligonucleotide composition, is not chirally controlled (stereorandom).

Linkage phosphorus of natural phosphate linkages is achiral. Linkage phosphorus of many modified internucleotidic linkages, e.g., phosphorothioate internucleotidic linkages, are chiral. In certain embodiments, during preparation of ds oligonucleotide compositions (e.g., in traditional phosphoramidite ds oligonucleotide synthesis), configurations of chiral linkage phosphorus are not purposefully designed or controlled, creating non-chirally controlled (stereorandom) ds oligonucleotide compositions (substantially racemic preparations) which are complex, random mixtures of various stereoisomers (diastereoisomers)—for ds oligonucleotides with n chiral internucleotidic linkages (linkage phosphorus being chiral), typically 2ⁿ stereoisomers (e.g., when n is 10, 2¹⁰=1,032; when n is 20, 2²⁰=1,048,576). These stereoisomers have the same constitution, but differ with respect to the pattern of stereochemistry of their linkage phosphorus.

In certain embodiments, stereorandom ds oligonucleotide compositions have sufficient properties and/or activities for certain purposes and/or applications. In certain embodiments, stereorandom ds oligonucleotide compositions can be cheaper, easier and/or simpler to produce than chirally controlled ds oligonucleotide compositions. However, stereoisomers within stereorandom compositions may have different properties, activities, and/or toxicities, resulting in inconsistent therapeutic effects and/or unintended side effects by stereorandom compositions, particularly compared to certain chirally controlled ds oligonucleotide compositions of ds oligonucleotides of the same constitution.

### 2.3.1. Chirally Controlled Double Stranded Oligonucleotide Compositions

In certain embodiments, the present disclosure encompasses technologies for designing and preparing chirally controlled ds oligonucleotide compositions. In certain embodiments, a chirally controlled ds oligonucleotide composition comprises a controlled/pre-determined (not random as in stereorandom compositions) level of a plurality of ds oligonucleotides, wherein the ds oligonucleotides share the same linkage phosphorus stereochemistry at one or more chiral internucleotidic linkages (chirally controlled internucleotidic linkages). In certain embodiments, ds oligonucleotides of a plurality share the same pattern of backbone chiral centers (stereochemistry of linkage phosphorus). In certain embodiments, a pattern of backbone chiral centers is as described in the present disclosure. In certain embodiments, ds oligonucleotides of a plurality share a common constitution. In certain embodiments, they are structurally identical.

For example, in certain embodiments, the present disclosure provides a ds oligonucleotide composition comprising a plurality of ds oligonucleotides, wherein ds oligonucleotides of the plurality share:
1) a common base sequence, and
2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”); wherein level of ds oligonucleotides of the plurality in the composition is non-random (e.g., controlled/pre-determined as described herein).

In certain embodiments, the present disclosure provides a ds oligonucleotide composition comprising a plurality of ds oligonucleotides, wherein ds oligonucleotides of the plurality share:
1) a common base sequence, and
2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”); wherein the composition is enriched relative to a substantially racemic preparation of ds oligonucleotides sharing the common base sequence for oligonucleotides of the plurality.

In certain embodiments, the present disclosure provides a ds oligonucleotide composition comprising a plurality of ds oligonucleotides, wherein ds oligonucleotides of the plurality share:
1) a common base sequence, and
2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”); wherein about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all ds oligonucleotides in the composition that share the common base sequence are ds oligonucleotides of the plurality.

In certain embodiments, the percentage/level of the ds oligonucleotides of a plurality is or is at least (DS)^{nc}, wherein DS is 90%-100%, and nc is the number of chirally controlled internucleotidic linkages. In certain embodiments, nc is 5, 6, 7, 8, 9, 10 or more. In certain embodiments, a percentage/level is at least 10%.

In certain embodiments, a percentage/level is at least 20%. In certain embodiments, a percentage/level is at least 30%. In certain embodiments, a percentage/level is at least 40%. In certain embodiments, a percentage/level is at least 50%. In certain embodiments, a percentage/level is at least 60%. In certain embodiments, a percentage/level is at least 65%. In certain embodiments, a percentage/level is at least 70%. In certain embodiments, a percentage/level is at least 75%. In certain embodiments, a percentage/level is at least 80%. In certain embodiments, a percentage/level is at least 85%. In certain embodiments, a percentage/level is at least 90%. In certain embodiments, a percentage/level is at least 95%.

In certain embodiments, ds oligonucleotides of a plurality share a common pattern of backbone linkages. In certain embodiments, each ds oligonucleotide of a plurality independently has an internucleotidic linkage of a particular constitution (e.g., —O—P(O)(SH)—O—) or a salt form thereof (e.g., —O—P(O)(SNa)—O—) independently at each internucleotidic linkage site. In certain embodiments, internucleotidic linkages at each internucleotidic linkage site are of the same form. In certain embodiments, internucleotidic linkages at each internucleotidic linkage site are of different forms.

In certain embodiments, ds oligonucleotides of a plurality share a common constitution. In certain embodiments, ds oligonucleotides of a plurality are of the same form of a common constitution. In certain embodiments, ds oligonucleotides of a plurality are of two or more forms of a common constitution. In certain embodiments, ds oligonucleotides of a plurality are each independently of a particularly oligonucleotide or a pharmaceutically acceptable salt thereof, or of a ds oligonucleotide having the same constitution as the particularly ds oligonucleotide or a pharmaceutically acceptable salt thereof. In certain embodiments, about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all ds oligonucleotides in the composition that share a common constitution are ds oligonucleotides of the plurality. In certain embodiments, a percentage of a level is or is at least (DS)^{nc}, wherein DS is 90%-100%, and nc is the number of chirally controlled internucleotidic linkages. In certain embodiments, nc is 5, 6, 7, 8, 9, 10 or more. In certain embodiments, a level is at least 10%. In certain embodiments, a level is at least 20%. In certain embodiments, a level is at least 30%. In certain embodiments, a level is at least 40%. In certain embodiments, a level is at least 50%. In certain embodiments, a level is at least 60%. In certain embodiments, a level is at least 65%. In certain embodiments, a level is at least 70%. In certain embodiments, a level is at least 75%. In certain embodiments, a level is at least 80%. In certain embodiments, a level is at least 85%. In certain embodiments, a level is at least 90%. In certain embodiments, a level is at least 95%.

In certain embodiments, each phosphorothioate internucleotidic linkage is independently a chirally controlled internucleotidic linkage.

In certain embodiments, the present disclosure provides a chirally controlled ds oligonucleotide composition comprising a plurality of ds oligonucleotides of a particular ds oligonucleotide type characterized by:
a) a common base sequence;
b) a common pattern of backbone linkages;
c) a common pattern of backbone chiral centers; wherein the composition is enriched, relative to a substantially racemic preparation of ds oligonucleotides having the same common base sequence, for ds oligonucleotides of the particular oligonucleotide type.

In certain embodiments, the present disclosure provides a chirally controlled ds oligonucleotide composition comprising a plurality of ds oligonucleotides of a particular ds oligonucleotide type characterized by:
a) a common base sequence;
b) a common pattern of backbone linkages;
c) a common pattern of backbone chiral centers; wherein ds oligonucleotides of the plurality comprise at least one internucleotidic linkage comprising a common linkage phosphorus in the Sp configuration; wherein the composition is enriched, relative to a substantially racemic preparation of d oligonucleotides having the same common base sequence, for ds oligonucleotides of the particular ds oligonucleotide type.

Common patterns of backbone chiral centers, as appreciated by those skilled in the art, comprise at least one Rp or at least one Sp. Certain patterns of backbone chiral centers are illustrated in, e.g., Table 1.

In certain embodiments, a chirally controlled ds oligonucleotide composition is enriched, relative to a substantially racemic preparation of ds oligonucleotides share the same common base sequence and a common pattern of backbone linkages, for ds oligonucleotides of the particular ds oligonucleotide type.

In certain embodiments, ds oligonucleotides of a plurality, e.g., a particular ds oligonucleotide type, have a common pattern of backbone phosphorus modifications and a common pattern of nucleoside modifications. In certain embodiments, ds oligonucleotides of a plurality have a common pattern of sugar modifications. In certain embodiments, ds oligonucleotides of a plurality have a common pattern of base modifications. In certain embodiments, ds oligonucleotides of a plurality have a common pattern of nucleoside modifications. In certain embodiments, ds oligonucleotides of a plurality have the same constitution. In certain embodiments, ds oligonucleotides of a plurality are identical. In certain embodiments, ds oligonucleotides of a plurality are of the same ds oligonucleotide (as those skilled in the art will appreciate, such ds oligonucleotides may each independently exist in one of the various forms of the ds oligonucleotide, and may be the same, or different forms of the ds oligonucleotide). In certain embodiments, ds oligonucleotides of a plurality are each independently of the same ds oligonucleotide or a pharmaceutically acceptable salt thereof.

In certain embodiments, the present disclosure provides chirally controlled ds oligonucleotide compositions, e.g., of many oligonucleotides in Table 1, whose “stereochemistry/linkage” contain S and/or R. In certain embodiments, ds oligonucleotides of a plurality are each independently a particular ds oligonucleotide in Table 1 whose “stereochemistry/linkage” contains S and/or R, optionally in various forms. In certain embodiments, ds oligonucleotides of a plurality are each independently a particular ds oligonucleotide in Table 1, whose “stereochemistry/linkage” contains S and/or R, or a pharmaceutically acceptable salt thereof.

In certain embodiments, level of a plurality of ds oligonucleotides in a composition can be determined as the product of the diastereopurity of each chirally controlled internucleotidic linkage in the ds oligonucleotides. In certain embodiments, diastereopurity of an internucleotidic linkage connecting two nucleosides in a ds oligonucleotide (or nucleic acid) is represented by the diastereopurity of an internucleotidic linkage of a dimer connecting the same two nucleosides, wherein the dimer is prepared using comparable conditions, in some instances, identical synthetic cycle conditions.

In certain embodiments, all chiral internucleotidic linkages are independently chiral controlled, and the composition is a completely chirally controlled ds oligonucleotide composition. In certain embodiments, not all chiral internucleotidic linkages are chiral controlled internucleotidic linkages, and the composition is a partially chirally controlled ds oligonucleotide composition.

Ds oligonucleotides may comprise or consist of various patterns of backbone chiral centers (patterns of stereochemistry of chiral linkage phosphorus). Certain useful patterns of backbone chiral centers are described in the present disclosure. In certain embodiments, a plurality of ds oligonucleotides share a common pattern of backbone chiral centers, which is or comprises a pattern described in the present disclosure (e.g., as in “Stereochemistry and Patterns of Backbone Chiral Centers”, a pattern of backbone chiral centers of a chirally controlled ds oligonucleotide in Table 1, etc.).

In certain embodiments, a chirally controlled ds oligonucleotide composition is chirally pure (or stereopure, stereochemically pure) ds oligonucleotide composition, wherein the ds oligonucleotide composition comprises a plurality of ds oligonucleotides, wherein the ds oligonucleotides are independently of the same stereoisomer (including that each chiral element of the ds oligonucleotides, including each chiral linkage phosphorus, is independently defined (stereodefined)). A chirally pure (or stereopure, stereochemically pure) ds oligonucleotide composition of a ds oligonucleotide stereoisomer does not contain other stereoisomers (as appreciated by those skilled in the art, one or more unintended stereoisomers may exist as impurities from, e.g., preparation, storage, etc.).

### 2.3.2 Stereochemistry and Patterns of Backbone Chiral Centers

In contrast to natural phosphate linkages, linkage phosphorus of chiral modified internucleotidic linkages, e.g., phosphorothioate internucleotidic linkages, are chiral. Among other things, the present disclosure provides technologies (e.g., oligonucleotides, compositions, methods, etc.) comprising control of stereochemistry of chiral linkage phosphorus in chiral internucleotidic linkages. In certain embodiments, as demonstrated herein, control of stereochemistry can provide improved properties and/or activities, including desired stability, reduced toxicity, improved reduction of target nucleic acids, etc. In certain embodiments, the present disclosure provides useful patterns of backbone chiral centers for oligonucleotides and/or regions thereof, which pattern is a combination of stereochemistry of each chiral linkage phosphorus (Rp or Sp) of chiral linkage phosphorus, indication of each achiral linkage phosphorus (Op, if any), etc. from 5′ to 3′. In certain embodiments, patterns of backbone chiral centers can control cleavage patterns of target nucleic acids when they are contacted with provided ds oligonucleotides or compositions thereof in a cleavage system (e.g., in vitro assay, cells, tissues, organs, organisms, subjects, etc.). In certain embodiments, patterns of backbone chiral centers improve cleavage efficiency and/or selectivity of target nucleic acids when they are contacted with provided ds oligonucleotides or compositions thereof in a cleavage system.

In certain embodiments, a pattern of backbone chiral centers of a ds oligonucleotide or a region thereof comprises or is any (Np)n(Op)m, wherein Np is Rp or Sp, Op represents a linkage phosphorus being achiral (e.g., as for the linkage phosphorus of natural phosphate linkages), and each of n and m is independently as defined and described in the present disclosure. In certain embodiments, a pattern of backbone chiral centers of a ds oligonucleotide or a region thereof comprises or is (Sp)n(Op)m, wherein each variable is independently as defined and described in the present disclosure. In certain embodiments, a pattern of backbone chiral centers of a ds oligonucleotide or a region thereof comprises or is (Rp)n(Op)m, wherein each variable is independently as defined and described in the present disclosure. In certain embodiments, n is 1. In certain embodiments, a pattern of backbone chiral centers of a ds oligonucleotide or a region thereof comprises or is (Sp)(Op)m, wherein m is 1, 2, 3, 4, 5, 6, 7, 8, 9, or 10. In certain embodiments, a pattern of backbone chiral centers of an oligonucleotide or a region thereof comprises or is (Rp)(Op)m, wherein m is 1, 2, 3, 4, 5, 6, 7, 8, 9, or 10. In certain embodiments, the pattern of backbone chiral centers of a 5′-wing is or comprises (Np)n(Op)m. In certain embodiments, the pattern of backbone chiral centers of a 5′-wing is or comprises (Sp)n(Op)m. In certain embodiments, the pattern of backbone chiral centers of a 5′-wing is or comprises (Rp)n(Op)m. In certain embodiments, the pattern of backbone chiral centers of a 5′-wing is or comprises (Sp)(Op)m. In certain embodiments, the pattern of backbone chiral centers of a 5′-wing is or comprises (Rp)(Op)m. In certain embodiments, the pattern of backbone chiral centers of a 5′-wing is (Sp)(Op)m. In certain embodiments, the pattern of backbone chiral centers of a 5′-wing is (Rp)(Op)m. In certain embodiments, the pattern of backbone chiral centers of a 5′-wing is (Sp)(Op)m, wherein Sp is the linkage phosphorus configuration of the first internucleotidic linkage of the oligonucleotide from the 5′-end. In certain embodiments, the pattern of backbone chiral centers of a 5′-wing is (Rp)(Op)m, wherein Rp is the linkage phosphorus configuration of the first internucleotidic linkage of the oligonucleotide from the 5′-end. In certain embodiments, as described in the present disclosure, m is 2; in certain embodiments, m is 3; in certain embodiments, m is 4; in certain embodiments, m is 5; in certain embodiments, m is 6.

In certain embodiments, a pattern of backbone chiral centers of a ds oligonucleotide or a region thereof comprises or is (Op)m(Np)n, wherein Np is Rp or Sp, Op represents a linkage phosphorus being achiral (e.g., as for the linkage phosphorus of natural phosphate linkages), and each of n and m is independently as defined and described in the present disclosure. In certain embodiments, a pattern of backbone chiral centers of an oligonucleotide or a region thereof comprises or is (Op)m(Sp)n, wherein each variable is independently as defined and described in the present disclosure. In certain embodiments, a pattern of backbone chiral centers of a ds oligonucleotide or a region thereof comprises or is (Op)m(Rp)n, wherein each variable is independently as defined and described in the present disclosure. In certain embodiments, n is 1. In certain embodiments, a pattern of backbone chiral centers of a ds oligonucleotide or a region thereof comprises or is (Op)m(Sp), wherein m is 1, 2, 3, 4, 5, 6, 7, 8, 9, or 10. In certain embodiments, a pattern of backbone chiral centers of an oligonucleotide or a region thereof comprises or is (Op)m(Rp), wherein m is 1, 2, 3, 4, 5, 6, 7, 8, 9, or 10. In certain embodiments, the pattern of backbone chiral centers of a 3′-wing is or comprises (Op)m(Np)n. In certain embodiments, the pattern of backbone chiral centers of a 3′-wing is or comprises (Op)m(Sp)n. In certain embodiments, the pattern of backbone chiral centers of a 3′-wing is or comprises (Op)m(Rp)n. In certain embodiments, the pattern of backbone chiral centers of a 3′-wing is or comprises (Op)m(Sp). In certain embodiments, the pattern of backbone chiral centers of a 3′-wing is or comprises (Op)m(Rp). In certain embodiments, the pattern of backbone chiral centers of a 3′-wing is (Op)m(Sp). In certain embodiments, the pattern of backbone chiral centers of a 3′-wing is (Op)m(Rp). In certain embodiments, the pattern of backbone chiral centers of a 3′-wing is (Op)m(Sp), wherein Sp is the linkage phosphorus configuration of the last internucleotidic linkage of the ds oligonucleotide from the 5′-end. In certain embodiments, the pattern of backbone chiral centers of a 3′-wing is (Op)m(Rp), wherein Rp is the linkage phosphorus configuration of the last internucleotidic linkage of the oligonucleotide from the 5′-end. In certain embodiments, as described in the present disclosure, m is 2; in certain embodiments, m is 3; in certain embodiments, m is 4; in certain embodiments, m is 5; in certain embodiments, m is 6.

In certain embodiments, a pattern of backbone chiral centers of a ds oligonucleotide or a region thereof (e.g., a core) comprises or is (Sp)m(Rp/Op)n or (Rp/Op)n(Sp)m, wherein each variable is independently as described in the present disclosure. In certain embodiments, a pattern of backbone chiral centers of a ds oligonucleotide or a region thereof (e.g., a core) comprises or is (Sp)m(Rp)n or (Rp)n(Sp)m, wherein each variable is independently as described in the present disclosure. In certain embodiments, a pattern of backbone chiral centers of a ds oligonucleotide or a region thereof (e.g., a core) comprises or is (Sp)m(Op)n or (Op)n(Sp)m, wherein each variable is independently as described in the present disclosure. In certain embodiments, a pattern of backbone chiral centers of a ds oligonucleotide or a region thereof (e.g., a core) comprises or is (Np)t[(Rp/Op)n(Sp)m]y or [(Rp/Op)n(Sp)m]y(Np)t, wherein y is 1-50, and each other variable is independently as described in the present disclosure. In certain embodiments, a pattern of backbone chiral centers of a ds oligonucleotide or a region thereof (e.g., a core) comprises or is (Np)t[(Rp)n(Sp)m]y or [(Rp)n(Sp)m]y(Np)t, wherein each variable is independently as described in the present disclosure. In certain embodiments, a pattern of backbone chiral centers of a a ds n oligonucleotide or a region thereof (e.g., a core) comprises or is [(Rp/Op)n(Sp)m]y(Rp)k, [(Rp/Op)n(Sp)m]y, (Sp)t[(Rp/Op)n(Sp)m]y, (Sp)t[(Rp/Op)n(Sp)m]y(Rp)k, wherein k is 1-50, and each other variable is independently as described in the present disclosure. In certain embodiments, a pattern of backbone chiral centers of a ds oligonucleotide or a region thereof (e.g., a core) comprises or is [(Op)n(Sp)m]y(Rp)k, [(Op)n(Sp)m]y, (Sp)t[(Op)n(Sp)m]y, (Sp)t[(Op)n(Sp)m]y(Rp)k, wherein each variable is independently as described in the present disclosure. In certain embodiments, a pattern of backbone chiral centers of a ds oligonucleotide or a region thereof (e.g., a core) comprises or is [(Rp)n(Sp)m]y(Rp)k, [(Rp)n(Sp)m]y, (Sp)t[(Rp)n(Sp)m]y, (Sp)t[(Rp)n(Sp)m]y(Rp)k, wherein each variable is independently as described in the present disclosure. In certain embodiments, an oligonucleotide comprises a core region. In certain embodiments, an oligonucleotide comprises a core region, wherein each sugar in the core region does not contain a 2′-OR¹, wherein R¹ is as described in the present disclosure. In certain embodiments, a ds oligonucleotide comprises a core region, wherein each sugar in the core region is independently a natural DNA sugar. In certain embodiments, the pattern of backbone chiral centers of the core comprises or is (Rp)(Sp)m. In certain embodiments, the pattern of backbone chiral centers of the core comprises or is (Op)(Sp)m. In certain embodiments, the pattern of backbone chiral centers of the core comprises or is (Np)t[(Rp/Op)n(Sp)m]y or [(Rp/Op)n(Sp)m]y(Np)t. In certain embodiments, the pattern of backbone chiral centers of the core comprises or is (Np)t[(Rp/Op)n(Sp)m]y or [(Rp/Op)n(Sp)m]y(Np)t. In certain embodiments, the pattern of backbone chiral centers of the core comprises or is (Np)t[(Rp)n(Sp)m]y or [(Rp)n(Sp)m]y(Np)t. In certain embodiments, the pattern of backbone chiral centers of a core comprises or is [(Rp/Op)n(Sp)m]y(Rp)k, [(Rp/Op)n(Sp)m]y, (Sp)t[(Rp/Op)n(Sp)m]y, (Sp)t[(Rp/Op)n(Sp)m]y(Rp)k. In certain embodiments, a pattern of backbone chiral centers of a core comprises or is [(Op)n(Sp)m]y(Rp)k, [(Op)n(Sp)m]y, (Sp)t[(Op)n(Sp)m]y, (Sp)t[(Op)n(Sp)m]y(Rp)k. In certain embodiments, a pattern of backbone chiral centers of a core comprises or is [(Rp)n(Sp)m]y(Rp)k, [(Rp)n(Sp)m]y, (Sp)t[(Rp)n(Sp)m]y, or (Sp)t[(Rp)n(Sp)m]y(Rp)k. In certain embodiments, a pattern of backbone chiral centers of a core comprises [(Rp)n(Sp)m]y(Rp)k. In certain embodiments, a pattern of backbone chiral centers of a core comprises [(Rp)n(Sp)m]y(Rp). In certain embodiments, a pattern of backbone chiral centers of a core comprises [(Rp)n(Sp)m]y. In certain embodiments, a pattern of backbone chiral centers of a core comprises (Sp)t[(Rp)n(Sp)m]y. In certain embodiments, a pattern of backbone chiral centers of a core comprises (Sp)t[(Rp)n(Sp)m]y(Rp)k. In certain embodiments, a pattern of backbone chiral centers of a core comprises (Sp)t[(Rp)n(Sp)m]y(Rp). In certain embodiments, a pattern of backbone chiral centers of a core is [(Rp)n(Sp)m]y(Rp)k. In certain embodiments, a pattern of backbone chiral centers of a core is [(Rp)n(Sp)m]y(Rp). In certain embodiments, a pattern of backbone chiral centers of a core is [(Rp)n(Sp)m]y. In certain embodiments, a pattern of backbone chiral centers of a core is (Sp)t[(Rp)n(Sp)m]y. In certain embodiments, a pattern of backbone chiral centers of a core is (Sp)t[(Rp)n(Sp)m]y(Rp)k. In certain embodiments, a pattern of backbone chiral centers of a core is (Sp)t[(Rp)n(Sp)m]y(Rp). In certain embodiments, each n is 1. In certain embodiments, each t is 1. In certain embodiments, t is 2, 3, 4, 5, 6, 7, 8, 9, or 10. In certain embodiments, each of t and n is 1. In certain embodiments, each m is 2 or more. In certain embodiments, k is 1. In certain embodiments, k is 2-10.

In certain embodiments, a pattern of backbone chiral centers comprises or is (Sp)m(Rp)n, (Rp)n(Sp)m, (Np)t(Rp)n(Sp)m, (Sp)t(Rp)n(Sp)m, (Np)t[(Rp)n(Sp)m]2, (Sp)t[(Rp)n(Sp)m]2, (Np)t(Op)n(Sp)m, (Sp)t(Op)n(Sp)m, (Np)t[(Op)n(Sp)m]2, or (Sp)t[(Op)n(Sp)m]2. In certain embodiments, a pattern is (Np)t(Op/Rp)n(Sp)m(Op/Rp)n(Sp)m. In certain embodiments, a pattern is (Np)t(Op/Rp)n(Sp)1-5(Op/Rp)n(Sp)m. In certain embodiments, a pattern is (Np)t(Op/Rp)n(Sp)2-5(Op/Rp)n(Sp)m. In certain embodiments, a pattern is (Np)t(Op/Rp)n(Sp)2(Op/Rp)n(Sp)m. In certain embodiments, a pattern is (Np)t(Op/Rp)n(Sp)3(Op/Rp)n(Sp)m. In certain embodiments, a pattern is (Np)t(Op/Rp)n(Sp)4(Op/Rp)n(Sp)m. In certain embodiments, a pattern is (Np)t(Op/Rp)n(Sp)5(Op/Rp)n(Sp)m.

In certain embodiments, Np is Sp. In certain embodiments, (Op/Rp) is Op. In certain embodiments, (Op/Rp) is Rp. In certain embodiments, Np is Sp and (Op/Rp) is Rp. In certain embodiments, Np is Sp and (Op/Rp) is Op. In certain embodiments, Np is Sp and at least one (Op/Rp) is Rp, and at least one (Op/Rp) is Op. In certain embodiments, a pattern of backbone chiral centers comprises or is (Rp)n(Sp)m, (Np)t(Rp)n(Sp)m, or (Sp)t(Rp)n(Sp)m, wherein m>2. In certain embodiments, a pattern of backbone chiral centers comprises or is (Rp)n(Sp)m, (Np)t(Rp)n(Sp)m, or (Sp)t(Rp)n(Sp)m, wherein n is 1, at least one t>1, and at least one m>2.

In certain embodiments, oligonucleotides comprising core regions whose patterns of backbone chiral centers starting with Rp can provide high activities and/or improved properties. In certain embodiments, oligonucleotides comprising core regions whose patterns of backbone chiral centers ending with Rp can provide high activities and/or improved properties. In certain embodiments, oligonucleotides comprising core regions whose patterns of backbone chiral centers starting with Rp provide high activities (e.g., target cleavage) without significantly impacting its properties, e.g., stability. In certain embodiments, oligonucleotides comprising core regions whose patterns of backbone chiral centers ending with Rp provide high activities (e.g., target cleavage) without significantly impacting its properties, e.g., stability. In certain embodiments, patterns of backbone chiral centers start with Rp and end with Sp. In certain embodiments, patterns of backbone chiral centers start with Rp and end with Rp. In certain embodiments, patterns of backbone chiral centers start with Sp and end with Rp.

In certain embodiments, a pattern of backbone chiral centers of a RNAi oligonucleotide or a region thereof (e.g., a core) comprises or is (Op)[(Rp/Op)n(Sp)m]y(Rp)k(Op), (Op)[(Rp/Op)n(Sp)m]y(Op), (Op)(Sp)t[(Rp/Op)n(Sp)m]y(Op), or (Op)(Sp)t[(Rp/Op)n(Sp)m]y(Rp)k(Op), wherein k is 1-50, and each other variable is independently as described in the present disclosure. In certain embodiments, a pattern of backbone chiral centers of a RNAi oligonucleotide comprises or is (Op)[(Rp/Op)n(Sp)m]y(Rp)k(Op), (Op)[(Rp/Op)n(Sp)m]y(Op), (Op)(Sp)t[(Rp/Op)n(Sp)m]y(Op), or (Op)(Sp)t[(Rp/Op)n(Sp)m]y(Rp)k(Op), wherein each of f, g, h and j is independently 1-50, and each other variable is independently as described in the present disclosure, and the oligonucleotide comprises a core region whose pattern of backbone chiral centers comprises or is [(Rp/Op)n(Sp)m]y(Rp)k, [(Rp/Op)n(Sp)m]y, (Sp)t[(Rp/Op)n(Sp)m]y, or (Sp)t[(Rp/Op)n(Sp)m]y(Rp)k as described in the present disclosure. In certain embodiments, a pattern of backbone chiral centers is or comprises (Op)[(Rp/Op)n(Sp)m]y(Rp)k(Op). In certain embodiments, a pattern of backbone chiral centers is or comprises (Op)[(Rp/Op)n(Sp)m]y(Rp)(Op). In certain embodiments, a pattern of backbone chiral centers is or comprises (Op)[(Rp/Op)n(Sp)m]y(Op). In certain embodiments, a pattern of backbone chiral centers is or comprises (Op)(Sp)t[(Rp/Op)n(Sp)m]y(Op). In certain embodiments, a pattern of backbone chiral centers is or comprises (Op)(Sp)t[(Rp/Op)n(Sp)m]y(Rp)k(Op). In certain embodiments, a pattern of backbone chiral centers is or comprises (Op)(Sp)t[(Rp/Op)n(Sp)m]y(Rp)(Op). In certain embodiments, a pattern of backbone chiral centers is or comprises (Op)[(Rp)n(Sp)m]y(Rp)k(Op). In certain embodiments, a pattern of backbone chiral centers is or comprises (Op)[(Rp)n(Sp)m]y(Rp)(Op). In certain embodiments, a pattern of backbone chiral centers is or comprises (Op)[(Rp)n(Sp)m]y(Op). In certain embodiments, a pattern of backbone chiral centers is or comprises (Op)(Sp)t[(Rp)n(Sp)m]y(Op). In certain embodiments, a pattern of backbone chiral centers is or comprises (Op)(Sp)t[(Rp)n(Sp)m]y(Rp)k(Op). In certain embodiments, a pattern of backbone chiral centers is or comprises (Op)(Sp)t[(Rp)n(Sp)m]y(Rp)(Op). In certain embodiments, each n is 1. In certain embodiments, k is 1. In certain embodiments, k is 2-10.

In certain embodiments, a pattern of backbone chiral centers of a RNAi oligonucleotide or a region thereof (e.g., a core) comprises or is (Np)f(Op)g[(Rp/Op)n(Sp)m]y(Rp)k(Op)h(Np)j, (Np)f(Op)g[(Rp/Op)n(Sp)m]y(Op)h(Np)j, (Np)f(Op)g(Sp)t[(Rp/Op)n(Sp)m]y(Op)h(Np)j, or (Np)f(Op)g(Sp)t[(Rp/Op)n(Sp)m]y(Rp)k(Op)h(Np)j, wherein each of f, g, h and j is independently 1-50, and each other variable is independently as described in the present disclosure.

In certain embodiments, a pattern of backbone chiral centers of a RNAi oligonucleotide comprises or is (Np)f(Op)g[(Rp/Op)n(Sp)m]y(Rp)k(Op)h(Np)j, (Np)f(Op)g[(Rp/Op)n(Sp)m]y(Op)h(Np)j, (Np)f(Op)g(Sp)t[(Rp/Op)n(Sp)m]y(Op)h(Np)j, or (Np)f(Op)g(Sp)t[(Rp/Op)n(Sp)m]y(Rp)k(Op)h(Np)j, and the oligonucleotide comprises a core region whose pattern of backbone chiral centers comprises or is [(Rp/Op)n(Sp)m]y(Rp)k, [(Rp/Op)n(Sp)m]y, (Sp)t[(Rp/Op)n(Sp)m]y, or (Sp)t[(Rp/Op)n(Sp)m]y(Rp)k as described in the present disclosure.

In certain embodiments, a pattern of backbone chiral centers of a RNAi oligonucleotide is (Np)f(Op)g[(Rp/Op)n(Sp)m]y(Rp)k(Op)h(Np)j, (Np)f(Op)g[(Rp/Op)n(Sp)m]y(Op)h(Np)j, (Np)f(Op)g(Sp)t[(Rp/Op)n(Sp)m]y(Op)h(Np)j, or (Np)f(Op)g(Sp)t[(Rp/Op)n(Sp)m]y(Rp)k(Op)h(Np)j, and the oligonucleotide comprises a core region whose pattern of backbone chiral centers comprises or is [(Rp/Op)n(Sp)m]y(Rp)k, [(Rp/Op)n(Sp)m]y, (Sp)t[(Rp/Op)n(Sp)m]y, or (Sp)t[(Rp/Op)n(Sp)m]y(Rp)k as described in the present disclosure. In certain embodiments, a pattern of backbone chiral centers is or comprises (Np)f(Op)g[(Rp/Op)n(Sp)m]y(Rp)k(Op)h(Np)j.

In certain embodiments, a pattern of backbone chiral centers is or comprises (Np)f(Op)g[(Rp/Op)n(Sp)m]y(Rp)(Op)h(Np)j. In certain embodiments, a pattern of backbone chiral centers is or comprises (Np)f(Op)g[(Rp/Op)n(Sp)m]y(Op)h(Np)j. In certain embodiments, a pattern of backbone chiral centers is or comprises (Np)f(Op)g(Sp)t[(Rp/Op)n(Sp)m]y(Op)h(Np)j. In certain embodiments, a pattern of backbone chiral centers is or comprises (Np)f(Op)g(Sp)t[(Rp/Op)n(Sp)m]y(Rp)k(Op)h(Np)j. In certain embodiments, a pattern of backbone chiral centers is or comprises (Np)f(Op)g(Sp)t[(Rp/Op)n(Sp)m]y(Rp)(Op)h(Np)j.

In certain embodiments, a pattern of backbone chiral centers is or comprises (Np)f(Op)g[(Rp)n(Sp)m]y(Rp)k(Op)h(Np)j. In certain embodiments, a pattern of backbone chiral centers is or comprises (Np)f(Op)g[(Rp)n(Sp)m]y(Rp)(Op)h(Np)j. In certain embodiments, a pattern of backbone chiral centers is or comprises (Np)f(Op)g[(Rp)n(Sp)m]y(Op)h(Np)j. In certain embodiments, a pattern of backbone chiral centers is or comprises (Np)f(Op)g(Sp)t[(Rp)n(Sp)m]y(Op)h(Np)j.

In certain embodiments, a pattern of backbone chiral centers is or comprises (Np)f(Op)g(Sp)t[(Rp)n(Sp)m]y(Rp)k(Op)h(Np)j. In certain embodiments, a pattern of backbone chiral centers is or comprises (Np)f(Op)g(Sp)t[(Rp)n(Sp)m]y(Rp)(Op)h(Np)j.

In certain embodiments, at least one Np is Sp. In certain embodiments, at least one Np is Rp. In certain embodiments, the 5′ most Np is Sp. In certain embodiments, the 3′ most Np is Sp. In certain embodiments, each Np is Sp. In certain embodiments, (Np)f(Op)g[(Rp/Op)n(Sp)m]y(Rp)k(Op)h(Np)j is (Sp)(Op)g[(Rp)n(Sp)m]y(Rp)k(Op)h(Sp).

In certain embodiments, (Np)f(Op)g[(Rp/Op)n(Sp)m]y(Rp)k(Op)h(Np)j is (Sp)(Op)g[(Rp)n(Sp)m]y(Rp)(Op)h(Sp). In certain embodiments, a pattern of backbone chiral center of a ds oligonucleotide is or comprises (Sp)(Op)g[(Rp)n(Sp)m]y(Rp)(Op)h(Sp). In certain embodiments, a pattern of backbone chiral center of a ds oligonucleotide is (Sp)(Op)g[(Rp)n(Sp)m]y(Rp)(Op)h(Sp). In certain embodiments, (Np)f(Op)g[(Rp/Op)n(Sp)m]y(Op)h(Np)j is (Sp)(Op)g[(Rp)n(Sp)m]y(Op)h(Sp). In certain embodiments, a pattern of backbone chiral center of a ds oligonucleotide is or comprises (Sp)(Op)g[(Rp)n(Sp)m]y(Op)h(Sp). In certain embodiments, a pattern of backbone chiral center of a ds oligonucleotide is (Sp)(Op)g[(Rp)n(Sp)m]y(Op)h(Sp).

In certain embodiments, (Np)f(Op)g(Sp)t[(Rp/Op)n(Sp)m]y(Op)h(Np)j is (Sp)(Op)g(Sp)t[(Rp)n(Sp)m]y(Op)h(Sp). In certain embodiments, a pattern of backbone chiral center of a ds oligonucleotide is or comprises (Sp)(Op)g(Sp)t[(Rp)n(Sp)m]y(Op)h(Sp). In certain embodiments, a pattern of backbone chiral center of a ds oligonucleotide is (Sp)(Op)g(Sp)t[(Rp)n(Sp)m]y(Op)h(Sp). In certain embodiments, (Np)f(Op)g(Sp)t[(Rp/Op)n(Sp)m]y(Rp)k(Op)h(Np)j is (Sp)(Op)g(Sp)t[(Rp)n(Sp)m]y(Rp)k(Op)h(Sp).

In certain embodiments, (Np)f(Op)g(Sp)t[(Rp/Op)n(Sp)m]y(Rp)k(Op)h(Np)j is (Sp)(Op)g(Sp)t[(Rp)n(Sp)m]y(Rp)(Op)h(Sp). In certain embodiments, a pattern of backbone chiral center of a ds oligonucleotide is or comprises (Sp)(Op)g(Sp)t[(Rp)n(Sp)m]y(Rp)(Op)h(Sp). In certain embodiments, a pattern of backbone chiral center of a ds oligonucleotide is (Sp)(Op)g(Sp)t[(Rp)n(Sp)m]y(Rp)(Op)h(Sp). In certain embodiments, each n is 1. In certain embodiments, f is 1. In certain embodiments, g is 1. In certain embodiments, g is greater than 1. In certain embodiments, g is 2. In certain embodiments, g is 3. In certain embodiments, g is 4. In certain embodiments, g is 5. In certain embodiments, g is 6. In certain embodiments, g is 7. In certain embodiments, g is 8. In certain embodiments, g is 9. In certain embodiments, g is 10. In certain embodiments, h is 1. In certain embodiments, h is greater than 1. In certain embodiments, h is 2. In certain embodiments, h is 3. In certain embodiments, h is 4. In certain embodiments, h is 5. In certain embodiments, h is 6. In certain embodiments, h is 7. In certain embodiments, h is 8. In certain embodiments, h is 9. In certain embodiments, h is 10. In certain embodiments, j is 1. In certain embodiments, k is 1. In certain embodiments, k is 2-10.

In certain embodiments, a pattern of backbone chiral centers of a RNAi oligonucleotide or a region thereof (e.g., a core) comprises or is [(Rp/Op)n(Sp)m]y, (Sp)t[(Rp/Op)n(Sp)m]y, (Sp)t[(Rp/Op)n(Sp)m]yRp, [(Rp/Op)n(Sp)m]y(Rp)k, (Sp)t[(Rp/Op)n(Sp)m]y(Rp)k, (Sp)t[(Rp/Op)n(Sp)m]y(Rp)k(Op)h, (Sp)t[(Rp/Op)n(Sp)m]y(Rp)k(Op)h(Np)j, wherein each variable is independently as described in the present disclosure.

In certain embodiments, in a provided pattern of backbone chiral centers, at least one (Rp/Op) is Rp. In certain embodiments, at least one (Rp/Op) is Op. In certain embodiments, each (Rp/Op) is Rp. In certain embodiments, each (Rp/Op) is Op. In certain embodiments, at least one of [(Rp)n(Sp)m]y or [(Rp/Op)n(Sp)m]y of a pattern is RpSp. In certain embodiments, at least one of [(Rp)n(Sp)m]y or [(Rp/Op)n(Sp)m]y of a pattern is or comprises RpSpSp. In certain embodiments, at least one of [(Rp)n(Sp)m]y or [(Rp/Op)n(Sp)m]y in a pattern is RpSp, and at least one of [(Rp)n(Sp)m]y or [(Rp/Op)n(Sp)m]y in a pattern is or comprises RpSpSp. For example, in certain embodiments, [(Rp)n(Sp)m]y in a pattern is (RpSp)[(Rp)n(Sp)m]_{(y-1)}; in certain embodiments, [(Rp)n(Sp)m]y in a pattern is (RpSp)[RpSpSp(Sp)₍ₘ₋₂₎][(Rp)n(Sp)m]_{(y-2)}. In certain embodiments, (Sp)t[(Rp)n(Sp)m]y(Rp) is (Sp)t(RpSp)[(Rp)n(Sp)m]_{(y-l)}(Rp). In certain embodiments, (Sp)t[(Rp)n(Sp)m]y(Rp) is (Sp)t(RpSp)[RpSpSp(Sp)₍ₘ₋₂₎][(Rp)n(Sp)m]_{(y-2)}(Rp). In certain embodiments, each [(Rp/Op)n(Sp)m] is independently [Rp(Sp)m]. In certain embodiments, the first Sp of (Sp)t represents linkage phosphorus stereochemistry of the first internucleotidic linkage of a ds oligonucleotide from 5′ to 3′. In certain embodiments, the first Sp of (Sp)t represents linkage phosphorus stereochemistry of the first internucleotidic linkage of a region from 5′ to 3′, e.g., a core. In certain embodiments, the last Np of (Np)j represents linkage phosphorus stereochemistry of the last internucleotidic linkage of the oligonucleotide from 5′ to 3′. In certain embodiments, the last Np is Sp.

In certain embodiments, a pattern of backbone chiral centers of a ds oligonucleotide or a region (e.g., of a 5′-wing) is or comprises Sp(Op)₃. In certain embodiments, a pattern of backbone chiral centers of a ds oligonucleotide or a region (e.g., of a 5′-wing) is or comprises Rp(Op)₃. In certain embodiments, a pattern of backbone chiral centers of a ds oligonucleotide or a region (e.g., of a 3′-wing) is or comprises (Op)₃Sp. In certain embodiments, a pattern of backbone chiral centers of a ds oligonucleotide or a region (e.g., of a 3′-wing) is or comprises (Op)₃Rp. In certain embodiments, a pattern of backbone chiral centers of a ds oligonucleotide or a region (e.g., of a core) is or comprises Rp(Sp)₄Rp(Sp)₄Rp. In certain embodiments, a pattern of backbone chiral centers of a ds oligonucleotide or a region (e.g., of a core) is or comprises (Sp)₅Rp(Sp)₄Rp. In certain embodiments, a pattern of backbone chiral centers of a ds oligonucleotide or a region (e.g., of a core) is or comprises (Sp)₅Rp(Sp)₅. In certain embodiments, a pattern of backbone chiral centers of a ds oligonucleotide or a region (e.g., of a core) is or comprises Rp(Sp)₄Rp(Sp)₅. In certain embodiments, a pattern of backbone chiral centers of a ds oligonucleotide is or comprises Np(Op)₃Rp(Sp)₄Rp(Sp)₄Rp(Op)₃Np. In certain embodiments, a pattern of backbone chiral centers of a ds oligonucleotide is or comprises Np(Op)₃(Sp)₅Rp(Sp)₄Rp(Op)₃Np. In certain embodiments, a pattern of backbone chiral centers of a ds oligonucleotide is or comprises Np(Op)₃(Sp)₅Rp(Sp)₅(Op)₃Np. In certain embodiments, a pattern of backbone chiral centers of a ds oligonucleotide is or comprises Np(Op)₃Rp(Sp)₄Rp(Sp)₅(Op)₃Np. In certain embodiments, a pattern of backbone chiral centers of a ds oligonucleotide is or comprises Sp(Op)₃Rp(Sp)₄Rp(Sp)₄Rp(Op)₃Sp. In certain embodiments, a pattern of backbone chiral centers of a ds oligonucleotide is or comprises Sp(Op)₃(Sp)₅Rp(Sp)₄Rp(Op)₃Sp. In certain embodiments, a pattern of backbone chiral centers of a ds oligonucleotide is or comprises Sp(Op)₃(Sp)₅Rp(Sp)₅(Op)₃Sp. In certain embodiments, a pattern of backbone chiral centers of a ds oligonucleotide is or comprises Sp(Op)₃Rp(Sp)₄Rp(Sp)₅(Op)₃Sp. In certain embodiments, a pattern of backbone chiral centers of a ds oligonucleotide is or comprises Rp(Op)₃Rp(Sp)₄Rp(Sp)₄Rp(Op)₃Rp. In certain embodiments, a pattern of backbone chiral centers of a ds oligonucleotide is or comprises Rp(Op)₃(Sp)₅Rp(Sp)₄Rp(Op)₃Rp. In certain embodiments, a pattern of backbone chiral centers of a ds oligonucleotide is or comprises Rp(Op)₃(Sp)₅Rp(Sp)₅(Op)₃Rp. In certain embodiments, a pattern of backbone chiral centers of a ds oligonucleotide is or comprises Rp(Op)₃Rp(Sp)₄Rp(Sp)₅(Op)₃Rp.

In certain embodiments, each of m, y, t, n, k, f, g, h, and j is independently 1-25.

In certain embodiments, m is 1-25. In certain embodiments, m is 1-20. In certain embodiments, m is 1-15. In certain embodiments, m is 1-10. In certain embodiments, m is 1-5. In certain embodiments, m is 2-20. In certain embodiments, m is 2-15. In certain embodiments, m is 2-10. In certain embodiments, m is 2-5. In certain embodiments, m is 1, 2, 3, 4, 5, 6, 7, 8, 9, 10, 11, 12, 13, 14, 15, 16, 17, 18, 19, 20, 21, 22, 23, 24, or 25. In certain embodiments, in a pattern of backbone chiral centers each m is independently 2 or more. In certain embodiments, each m is independently 2, 3, 4, 5, 6, 7, 8, 9, or 10. In certain embodiments, each m is independently 2-3, 2-5, 2-6, or 2-10. In certain embodiments, m is 2. In certain embodiments, m is 3. In certain embodiments, m is 4. In certain embodiments, m is 5. In certain embodiments, m is 6. In certain embodiments, m is 7. In certain embodiments, m is 8. In certain embodiments, m is 9. In certain embodiments, m is 10. In certain embodiments, where there are two or more occurrences of m, they can be the same or different, and each of them is independently as described in the present disclosure.

In certain embodiments, y is 1-25. In certain embodiments, y is 1-20. In certain embodiments, y is 1-15. In certain embodiments, y is 1-10. In certain embodiments, y is 1-5. In certain embodiments, y is 2-20. In certain embodiments, y is 2-15. In certain embodiments, y is 2-10. In certain embodiments, y is 2-5. In certain embodiments, y is 1, 2, 3, 4, 5, 6, 7, 8, 9, 10, 11, 12, 13, 14, 15, 16, 17, 18, 19, 20, 21, 22, 23, 24, or 25. In certain embodiments, y is 1, 2, 3, 4, 5, 6, 7, 8, 9, or 10. In certain embodiments, y is 1. In certain embodiments, y is 2. In certain embodiments, y is 3. In certain embodiments, y is 4. In certain embodiments, y is 5. In certain embodiments, y is 6. In certain embodiments, y is 7. In certain embodiments, y is 8. In certain embodiments, y is 9. In certain embodiments, y is 10.

In certain embodiments, t is 1-25. In certain embodiments, t is 1-20. In certain embodiments, t is 1-15. In certain embodiments, t is 1-10. In certain embodiments, t is 1-5. In certain embodiments, t is 2-20. In certain embodiments, t is 2-15. In certain embodiments, t is 2-10. In certain embodiments, t is 2-5. In certain embodiments, t is 1, 2, 3, 4, 5, 6, 7, 8, 9, 10, 11, 12, 13, 14, 15, 16, 17, 18, 19, 20, 21, 22, 23, 24, or 25. In certain embodiments, each t is independently 1, 2, 3, 4, 5, 6, 7, 8, 9, or 10. In certain embodiments, t is 2 or more. In certain embodiments, t is 1. In certain embodiments, t is 2. In certain embodiments, t is 3. In certain embodiments, t is 4. In certain embodiments, t is 5. In certain embodiments, t is 6. In certain embodiments, t is 7. In certain embodiments, t is 8. In certain embodiments, t is 9. In certain embodiments, t is 10. In certain embodiments, where there are two or more occurrences of t, they can be the same or different, and each of them is independently as described in the present disclosure.

In certain embodiments, n is 1-25. In certain embodiments, n is 1, 2, 3, 4, 5, 6, 7, 8, 9, 10, 11, 12, 13, 14, 15, 16, 17, 18, 19, 20, 21, 22, 23, 24, or 25. In certain embodiments, n is 1. In certain embodiments, n is 2. In certain embodiments, n is 3. In certain embodiments, n is 4. In certain embodiments, n is 5. In certain embodiments, n is 6. In certain embodiments, n is 7. In certain embodiments, n is 8. In certain embodiments, n is 9. In certain embodiments, n is 10. In certain embodiments, where there are two or more occurrences of n, they can be the same or different, and each of them is independently as described in the present disclosure. In certain embodiments, in a pattern of backbone chiral centers, at least one occurrence of n is 1; in some cases, each n is 1.

In certain embodiments, k is 1-25. In certain embodiments, k is 1, 2, 3, 4, 5, 6, 7, 8, 9, 10, 11, 12, 13, 14, 15, 16, 17, 18, 19, 20, 21, 22, 23, 24, or 25. In certain embodiments, k is 1. In certain embodiments, k is 2. In certain embodiments, k is 3. In certain embodiments, k is 4. In certain embodiments, k is 5. In certain embodiments, k is 6. In certain embodiments, k is 7. In certain embodiments, k is 8. In certain embodiments, k is 9. In certain embodiments, k is 10.

In certain embodiments, f is 1-25. In certain embodiments, f is 1-20. In certain embodiments, f is 1-10. In certain embodiments, f is 1-5. In certain embodiments, f is 1, 2, 3, 4, 5, 6, 7, 8, 9, 10, 11, 12, 13, 14, 15, 16, 17, 18, 19, 20, 21, 22, 23, 24, or 25. In certain embodiments, f is 1. In certain embodiments, f is 2. In certain embodiments, f is 3. In certain embodiments, f is 4. In certain embodiments, f is 5. In certain embodiments, f is 6. In certain embodiments, f is 7. In certain embodiments, f is 8. In certain embodiments, f is 9. In certain embodiments, f is 10.

In certain embodiments, g is 1-25. In certain embodiments, g is 1-20. In certain embodiments, g is 1-9. In certain embodiments, g is 1-5. In certain embodiments, g is 2-10. In certain embodiments, g is 2-5. In certain embodiments, g is 1, 2, 3, 4, 5, 6, 7, 8, 9, 10, 11, 12, 13, 14, 15, 16, 17, 18, 19, 20, 21, 22, 23, 24, or 25. In certain embodiments, g is 1. In certain embodiments, g is 2. In certain embodiments, g is 3. In certain embodiments, g is 4. In certain embodiments, g is 5. In certain embodiments, g is 6. In certain embodiments, g is 7. In certain embodiments, g is 8. In certain embodiments, g is 9. In certain embodiments, g is 10.

In certain embodiments, h is 1-25. In certain embodiments, h is 1-10. In certain embodiments, h is 1-5. In certain embodiments, h is 2-10. In certain embodiments, h is 2-5. In certain embodiments, h is 1, 2, 3, 4, 5, 6, 7, 8, 9, 10, 11, 12, 13, 14, 15, 16, 17, 18, 19, 20, 21, 22, 23, 24, or 25. In certain embodiments, h is 1. In certain embodiments, h is 2. In certain embodiments, h is 3. In certain embodiments, h is 4. In certain embodiments, h is 5. In certain embodiments, h is 6. In certain embodiments, h is 7. In certain embodiments, h is 8. In certain embodiments, h is 9. In certain embodiments, h is 10.

In certain embodiments, j is 1-25. In certain embodiments, j is 1-10. In certain embodiments, j is 1-5. In certain embodiments, j is 1, 2, 3, 4, 5, 6, 7, 8, 9, 10, 11, 12, 13, 14, 15, 16, 17, 18, 19, 20, 21, 22, 23, 24, or 25. In certain embodiments, j is 1. In certain embodiments, j is 2. In certain embodiments, j is 3. In certain embodiments, j is 4. In certain embodiments, j is 5. In certain embodiments, j is 6. In certain embodiments, j is 7. In certain embodiments, j is 8. In certain embodiments, j is 9. In certain embodiments, j is 10.

In certain embodiments, at least one n is 1, and at least one m is no less than 2. In certain embodiments, at least one n is 1, at least one t is no less than 2, and at least one m is no less than 3. In certain embodiments, each n is 1. In certain embodiments, t is 1. In certain embodiments, at least one t>1. In certain embodiments, at least one t>2. In certain embodiments, at least one t>3. In certain embodiments, at least one t>4. In certain embodiments, at least one m>1. In certain embodiments, at least one m>2. In certain embodiments, at least one m>3. In certain embodiments, at least one m>4. In certain embodiments, a pattern of backbone chiral centers comprises one or more achiral natural phosphate linkages. In certain embodiments, the sum of m, t, and n (or m and n if no t is in a pattern) is no less than 5, 6, 7, 8, 9, 10, 11, 12, 13, 14, 15, 16, 17, 18, 19 or 20. In certain embodiments, the sum is 5. In certain embodiments, the sum is 6. In certain embodiments, the sum is 7. In certain embodiments, the sum is 8. In certain embodiments, the sum is 9. In certain embodiments, the sum is 10. In certain embodiments, the sum is 11. In certain embodiments, the sum is 12. In certain embodiments, the sum is 13. In certain embodiments, the sum is 14. In certain embodiments, the sum is 15.

In certain embodiments, a number of linkage phosphorus in chirally controlled internucleotidic linkages are Sp. In certain embodiments, at least 10%, 20%, 25%, 30%, 35%, 40%, 45%, 50%, 55%, 60%, 65%, 70%, 75%, 80%, 85%, 90% or 95% of chirally controlled internucleotidic linkages have Sp linkage phosphorus. In certain embodiments, at least 10%, 20%, 25%, 30%, 35%, 40%, 45%, 50%, 55%, 60%, 65%, 70%, 75%, 80%, 85%, 90% or 95% of all chiral internucleotidic linkages are chirally controlled internucleotidic linkages having Sp linkage phosphorus. In certain embodiments, at least 10%, 20%, 25%, 30%, 35%, 40%, 45%, 50%, 55%, 60%, 65%, 70%, 75%, 80%, 85%, 90% or 95% of all internucleotidic linkages are chirally controlled internucleotidic linkages having Sp linkage phosphorus. In certain embodiments, the percentage is at least 20%. In certain embodiments, the percentage is at least 30%. In certain embodiments, the percentage is at least 40%. In certain embodiments, the percentage is at least 50%. In certain embodiments, the percentage is at least 60%.

In certain embodiments, the percentage is at least 65%. In certain embodiments, the percentage is at least 70%. In certain embodiments, the percentage is at least 75%. In certain embodiments, the percentage is at least 80%. In certain embodiments, the percentage is at least 90%. In certain embodiments, the percentage is at least 95%. In certain embodiments, at least 1, 2, 3, 4, 5, 6, 7, 8, 9, 10, 11, 12, 13, 14, 15, 16, 17, 18, 19, 20, 21, 22, 23, 24, or 25 internucleotidic linkages are chirally controlled internucleotidic linkages having Sp linkage phosphorus. In certain embodiments, at least 5 internucleotidic linkages are chirally controlled internucleotidic linkages having Sp linkage phosphorus. In certain embodiments, at least 6 internucleotidic linkages are chirally controlled internucleotidic linkages having Sp linkage phosphorus. In certain embodiments, at least 7 internucleotidic linkages are chirally controlled internucleotidic linkages having Sp linkage phosphorus. In certain embodiments, at least 8 internucleotidic linkages are chirally controlled internucleotidic linkages having Sp linkage phosphorus. In certain embodiments, at least 9 internucleotidic linkages are chirally controlled internucleotidic linkages having Sp linkage phosphorus. In certain embodiments, at least 10 internucleotidic linkages are chirally controlled internucleotidic linkages having Sp linkage phosphorus. In certain embodiments, at least 11 internucleotidic linkages are chirally controlled internucleotidic linkages having Sp linkage phosphorus. In certain embodiments, at least 12 internucleotidic linkages are chirally controlled internucleotidic linkages having Sp linkage phosphorus. In certain embodiments, at least 13 internucleotidic linkages are chirally controlled internucleotidic linkages having Sp linkage phosphorus. In certain embodiments, at least 14 internucleotidic linkages are chirally controlled internucleotidic linkages having Sp linkage phosphorus. In certain embodiments, at least 15 internucleotidic linkages are chirally controlled internucleotidic linkages having Sp linkage phosphorus. In certain embodiments, at least 1, 2, 3, 4, 5, 6, 7, 8, 9, 10, 11, 12, 13, 14, 15, 16, 17, 18, 19, 20, 21, 22, 23, 24, or 25 internucleotidic linkages are chirally controlled internucleotidic linkages having Rp linkage phosphorus. In certain embodiments, no more than 1, 2, 3, 4, 5, 6, 7, 8, 9, 10, 11, 12, 13, 14, 15, 16, 17, 18, 19, 20, 21, 22, 23, 24, or 25 internucleotidic linkages are chirally controlled internucleotidic linkages having Rp linkage phosphorus. In certain embodiments, one and no more than one internucleotidic linkage in a ds oligonucleotide is a chirally controlled internucleotidic linkage having Rp linkage phosphorus. In certain embodiments, 2 and no more than 2 internucleotidic linkages in a ds oligonucleotide are chirally controlled internucleotidic linkages having Rp linkage phosphorus. In certain embodiments, 3 and no more than 3 internucleotidic linkages in a ds oligonucleotide are chirally controlled internucleotidic linkages having Rp linkage phosphorus. In certain embodiments, 4 and no more than 4 internucleotidic linkages in a ds oligonucleotide are chirally controlled internucleotidic linkages having Rp linkage phosphorus. In certain embodiments, 5 and no more than 5 internucleotidic linkages in a ds oligonucleotide are chirally controlled internucleotidic linkages having Rp linkage phosphorus.

In certain embodiments, all, essentially all or most of the internucleotidic linkages in a ds oligonucleotide are in the Sp configuration (e.g., about 50%-100%, 55%-100%, 60%-100%, 65%-100%, 70%-100%, 75%-100%, 80%-100%, 85%-100%, 90%-100%, 55%-95%, 60%-95%, 65%-95%, or about 55%, 60%, 65%, 70%, 75%, 80%, 85%, 90%, 95%, 97%, 99% or more of all chirally controlled internucleotidic linkages, or of all chiral internucleotidic linkages, or of all internucleotidic linkages in the oligonucleotide) except for one or a minority of internucleotidic linkages (e.g., 1, 2, 3, 4, or 5, and/or less than 50%, 45%, 40%, 35%, 30%, 25%, 20%, 15%, 10%, or 5% of all chirally controlled internucleotidic linkages, or of all chiral internucleotidic linkages, or of all internucleotidic linkages in the oligonucleotide) being in the Rp configuration. In certain embodiments, all, essentially all or most of the internucleotidic linkages in a core are in the Sp configuration (e.g., about 50%-100%, 55%-100%, 60%-100%, 65%-100%, 70%-100%, 75%-100%, 80%-100%, 85%-100%, 90%-100%, 55%-95%, 60%-95%, 65%-95%, or about 55%, 60%, 65%, 70%, 75%, 80%, 85%, 90%, 95%, 97%, 99% or more of all chirally controlled internucleotidic linkages, or of all chiral internucleotidic linkages, or of all internucleotidic linkages, in the core) except for one or a minority of internucleotidic linkages (e.g., 1, 2, 3, 4, or 5, and/or less than 50%, 45%, 40%, 35%, 30%, 25%, 20%, 15%, 10%, or 5% of all chirally controlled internucleotidic linkages, or of all chiral internucleotidic linkages, or of all internucleotidic linkages, in the core) being in the Rp configuration. In certain embodiments, all, essentially all or most of the internucleotidic linkages in the core are a phosphorothioate in the Sp configuration (e.g., about 50%-100%, 55%-100%, 60%-100%, 65%-100%, 70%-100%, 75%-100%, 80%-100%, 85%-100%, 90%-100%, 55%-95%, 60%-95%, 65%-95%, or about 55%, 60%, 65%, 70%, 75%, 80%, 85%, 90%, 95%, 97%, 99% or more of all chirally controlled internucleotidic linkages, or of all chiral internucleotidic linkages, or of all internucleotidic linkages, in the core) except for one or a minority of internucleotidic linkages (e.g., 1, 2, 3, 4, or 5, and/or less than 50%, 45%, 40%, 35%, 30%, 25%, 20%, 15%, 10%, or 5% of all chirally controlled internucleotidic linkages, or of all chiral internucleotidic linkages, or of all internucleotidic linkages, in the core) being a phosphorothioate in the Rp configuration. In certain embodiments, each internucleotidic linkage in the core is a phosphorothioate in the Sp configuration except for one phosphorothioate in the Rp configuration. In certain embodiments, each internucleotidic linkage in the core is a phosphorothioate in the Sp configuration except for one phosphorothioate in the Rp configuration.

In certain embodiments, a ds oligonucleotide comprises one or more Rp internucleotidic linkages. In certain embodiments, a ds oligonucleotide comprises one and no more than one Rp internucleotidic linkages. In certain embodiments, a ds oligonucleotide comprises two or more Rp internucleotidic linkages. In certain embodiments, a ds oligonucleotide comprises three or more Rp internucleotidic linkages. In certain embodiments, a ds oligonucleotide comprises four or more Rp internucleotidic linkages. In certain embodiments, a ds oligonucleotide comprises five or more Rp internucleotidic linkages. In certain embodiments, about 5%-50% of all chirally controlled internucleotidic linkages in a ds oligonucleotide are Rp. In certain embodiments, about 5%-40% of all chirally controlled internucleotidic linkages in a ds oligonucleotide are Rp. In certain embodiments, about 10%-40% of all chirally controlled internucleotidic linkages in a ds oligonucleotide are Rp. In certain embodiments, about 15%-40% of all chirally controlled internucleotidic linkages in a ds oligonucleotide are Rp. In certain embodiments, about 20%-40% of all chirally controlled internucleotidic linkages in a ds oligonucleotide are Rp. In certain embodiments, about 25%-40% of all chirally controlled internucleotidic linkages in a ds oligonucleotide are Rp. In certain embodiments, about 30%-40% of all chirally controlled internucleotidic linkages in a ds oligonucleotide are Rp. In certain embodiments, about 35%-40% of all chirally controlled internucleotidic linkages in a ds oligonucleotide are Rp.

In certain embodiments, instead of an Rp internucleotidic linkage, a natural phosphate linkage may be similarly utilized, optionally with a modification, e.g., a sugar modification (e.g., a 5′-modification such as R^{5s} as described herein). In certain embodiments, a modification improves stability of a natural phosphate linkage.

In certain embodiments, the present disclosure provides a ds oligonucleotide having a pattern of backbone chiral centers as described herein. In certain embodiments, oligonucleotides in a chirally controlled ds oligonucleotide composition share a common pattern of backbone chiral centers as described herein.

In certain embodiments, at least about 25% of the internucleotidic linkages of a dsRNAi oligonucleotide are chirally controlled and have Sp linkage phosphorus. In certain embodiments, at least about 30% of the internucleotidic linkages of a ds oligonucleotide are chirally controlled and have Sp linkage phosphorus. In certain embodiments, at least about 40% of the internucleotidic linkages of a provided ds oligonucleotide are chirally controlled and have Sp linkage phosphorus. In certain embodiments, at least about 50% of the internucleotidic linkages of a provided ds oligonucleotide are chirally controlled and have Sp linkage phosphorus. In certain embodiments, at least about 60% of the internucleotidic linkages of a provided ds oligonucleotide are chirally controlled and have Sp linkage phosphorus. In certain embodiments, at least about 65% of the internucleotidic linkages of a provided ds oligonucleotide are chirally controlled and have Sp linkage phosphorus. In certain embodiments, at least about 70% of the internucleotidic linkages of a provided ds oligonucleotide are chirally controlled and have Sp linkage phosphorus. In certain embodiments, at least about 75% of the internucleotidic linkages of a provided ds oligonucleotide are chirally controlled and have Sp linkage phosphorus. In certain embodiments, at least about 80% of the internucleotidic linkages of a provided ds oligonucleotide are chirally controlled and have Sp linkage phosphorus. In certain embodiments, at least about 85% of the internucleotidic linkages of a provided ds oligonucleotide are chirally controlled and have Sp linkage phosphorus. In certain embodiments, at least about 90% of the internucleotidic linkages of a provided ds oligonucleotide are chirally controlled and have Sp linkage phosphorus. In certain embodiments, at least about 95% of the internucleotidic linkages of a provided ds oligonucleotide are chirally controlled and have Sp linkage phosphorus.

In certain embodiments, the present disclosure provides chirally controlled ds oligonucleotide compositions, e.g., chirally controlled dsRNAi oligonucleotide compositions, wherein the composition comprises a non-random or controlled level of a plurality of oligonucleotides, wherein oligonucleotides of the plurality share a common base sequence, and share the same configuration of linkage phosphorus independently at 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more chiral internucleotidic linkages.

In certain embodiments, dsRNAi oligonucleotides comprise 2-30 chirally controlled internucleotidic linkages. In certain embodiments, provided ds oligonucleotide compositions comprise 5-30 chirally controlled internucleotidic linkages. In certain embodiments, provided ds oligonucleotide compositions comprise 10-30 chirally controlled internucleotidic linkages.

In certain embodiments, a percentage is about 5%-100%. In certain embodiments, a percentage is at least 50%, 55%, 60%, 65%, 70%, 75%, 80%, 85%, 90%, 95%, 965, 96%, 98%, or 99%. In certain embodiments, a percentage is about 5%, 10%, 15%, 20%, 25%, 30%, 35%, 40%, 45%, 50%, 55%, 60%, 65%, 70%, 75%, 80%, 85%, 90%, 95%, 965, 96%, 98%, or 99%.

In certain embodiments, a pattern of backbone chiral centers in a dsRNAi oligonucleotide comprises a pattern of i^{o}-i^{s}-i^{o}-i^{s}-i^{o}, i^{o}-i^{s}-i^{s}-i^{s}-i^{o}, i^{o}-i^{s}-i^{s}-i^{s}-i^{o}-i^{s}, i^{s}-i^{o}-i^{s}-i^{o}, i^{s}-i^{o}-i^{s}-i^{o}, i^{s}-i^{o}-i^{s}-i^{o}-i^{s}, i^{s}-i^{o}i^{s}-i^{o}i^{s}-i^{o}, i^{s}-i^{o}i^{s}-i^{o}i^{s}-i^{o}i^{s}-i^{o}, i^{s}-i^{o}i^{s}-i^{s}i^{s}-i^{o}, i^{s}-i^{o}i^{s}-i^{s}i^{s}-i^{o}, i^{s}-i^{s}i^{o}-i^{s}i^{s}-i^{s}i^{s}-i^{o}i^{s}-i^{s}-i^{s}-i^{s}-i^{o}i^{s}-i^{o}-i^{s}-i^{s}-i^{s}-, i^{s}-i^{s}-i^{s}-i^{s}-i^{o}-i^{s}-i^{o}-i^{s}-i^{s}-i^{s}-i^{s}, i^{s}-i^{s}-i^{s}-i^{s}-i^{s}, i^{s}-i^{s}-i^{s}-i^{s}-i^{s}-i^{s}, i^{s}-i^{s}-i^{s}-i^{s}-i^{s}-i^{s}-i^{s}, i^{s}-i^{s}-i^{s}-i^{s}-i^{s}-i^{s}-i^{s}-i^{s}, i^{s}-i^{s}-i^{s}-i^{s}-i^{s}-i^{s}-i^{s}-i^{s}-i^{s}, or i^{r}-i^{r}-i^{r}, wherein is represents an internucleotidic linkage in the Sp configuration; i^{o} represents an achiral internucleotidic linkage; and i^{r} represents an internucleotidic linkage in the Rp configuration.

In certain embodiments, an internucleotidic linkage in the Sp configuration (having a Sp linkage phosphorus) is a phosphorothioate internucleotidic linkage. In certain embodiments, an achiral internucleotidic linkage is a natural phosphate linkage. In certain embodiments, an internucleotidic linkage in the Rp configuration (having a Rp linkage phosphorus) is a phosphorothioate internucleotidic linkage. In certain embodiments, each internucleotidic linkage in the Sp configuration is a phosphorothioate internucleotidic linkage. In certain embodiments, each achiral internucleotidic linkage is a natural phosphate linkage. In certain embodiments, each internucleotidic linkage in the Rp configuration is a phosphorothioate internucleotidic linkage. In certain embodiments, each internucleotidic linkage in the Sp configuration is a phosphorothioate internucleotidic linkage, each achiral internucleotidic linkage is a natural phosphate linkage, and each internucleotidic linkage in the Rp configuration is a phosphorothioate internucleotidic linkage.

In certain embodiments, dsRNAi oligonucleotides in chirally controlled oligonucleotide compositions each comprise different types of internucleotidic linkages. In certain embodiments, dsRNAi oligonucleotides comprise at least one natural phosphate linkage and at least one modified internucleotidic linkage. In certain embodiments, dsRNAi oligonucleotides comprise at least one natural phosphate linkage and at least two modified internucleotidic linkages. In certain embodiments, dsRNAi oligonucleotides comprise at least one natural phosphate linkage and at least three modified internucleotidic linkages. In certain embodiments, dsRNAi oligonucleotides comprise at least one natural phosphate linkage and at least four modified internucleotidic linkages. In certain embodiments, dsRNAi oligonucleotides comprise at least one natural phosphate linkage and at least five modified internucleotidic linkages. In certain embodiments, dsRNAi oligonucleotides comprise at least one natural phosphate linkage and 1, 2, 3, 4, 5, 6, 7, 8, 9, 10, 11, 12, 13, 14, 15, 16, 17, 18, 19, 20, 21, 22, 23, 24, or 25 modified internucleotidic linkages. In certain embodiments, a modified internucleotidic linkage is a phosphorothioate internucleotidic linkage. In certain embodiments, each modified internucleotidic linkage is a phosphorothioate internucleotidic linkage. In certain embodiments, a modified internucleotidic linkage is a phosphorothioate triester internucleotidic linkage. In certain embodiments, each modified internucleotidic linkage is a phosphorothioate triester internucleotidic linkage. In certain embodiments, RNAi oligonucleotides comprise at least one natural phosphate linkage and at least 2, 3, 4, 5, 6, 7, 8, 9, 10, 11, 12, 13, 14, 15, 16, 17, 18, 19, 20, 21, 22, 23, 24, or 25 consecutive modified internucleotidic linkages. In certain embodiments, RNAi oligonucleotides comprise at least one natural phosphate linkage and at least 2, 3, 4, 5, 6, 7, 8, 9, 10, 11, 12, 13, 14, 15, 16, 17, 18, 19, 20, 21, 22, 23, 24, or 25 consecutive phosphorothioate internucleotidic linkages. In certain embodiments, dsRNAi oligonucleotides comprise at least one natural phosphate linkage and at least 2, 3, 4, 5, 6, 7, 8, 9, 10, 11, 12, 13, 14, 15, 16, 17, 18, 19, 20, 21, 22, 23, 24, or 25 consecutive phosphorothioate triester internucleotidic linkages.

In certain embodiments, oligonucleotides in a chirally controlled ds oligonucleotide composition each comprise at least two internucleotidic linkages that have different stereochemistry and/or different P-modifications relative to one another. In certain embodiments, at least two internucleotidic linkages have different stereochemistry relative to one another, and the ds oligonucleotides each comprise a pattern of backbone chiral centers comprising alternating linkage phosphorus stereochemistry.

In certain embodiments, a linkage comprises a chiral auxiliary, which, for example, is used to control the stereoselectivity of a reaction, e.g., a coupling reaction in a ds oligonucleotide synthesis cycle. In certain embodiments, a phosphorothioate triester linkage does not comprise a chiral auxiliary. In certain embodiments, a phosphorothioate triester linkage is intentionally maintained until and/or during the administration of the oligonucleotide composition to a subject.

In certain embodiments, purity, particularly stereochemical purity, and particularly diastereomeric purity of many ds oligonucleotides and compositions thereof wherein all other chiral centers in the ds oligonucleotides but the chiral linkage phosphorus centers have been stereodefined (e.g., carbon chiral centers in the sugars, which are defined in, e.g., phosphoramidites for ds oligonucleotide synthesis), can be controlled by stereoselectivity (as appreciated by those skilled in this art, diastereoselectivity in many cases of ds oligonucleotide synthesis wherein the ds oligonucleotide comprise more than one chiral centers) at chiral linkage phosphorus in coupling steps when forming chiral internucleotidic linkages. In certain embodiments, a coupling step has a stereoselectivity (diastereoselectivity when there are other chiral centers) of 60% at the linkage phosphorus. After such a coupling step, the new internucleotidic linkage formed may be referred to have a 60% stereochemical purity (for ds oligonucleotides, typically diastereomeric purity in view of the existence of other chiral centers). In certain embodiments, each coupling step independently has a stereoselectivity of at least 60%. In certain embodiments, each coupling step independently has a stereoselectivity of at least 70%. In certain embodiments, each coupling step independently has a stereoselectivity of at least 80%. In certain embodiments, each coupling step independently has a stereoselectivity of at least 85%. In certain embodiments, each coupling step independently has a stereoselectivity of at least 90%. In certain embodiments, each coupling step independently has a stereoselectivity of at least 91%. In certain embodiments, each coupling step independently has a stereoselectivity of at least 92%. In certain embodiments, each coupling step independently has a stereoselectivity of at least 93%. In certain embodiments, each coupling step independently has a stereoselectivity of at least 94%. In certain embodiments, each coupling step independently has a stereoselectivity of at least 95%. In certain embodiments, each coupling step independently has a stereoselectivity of at least 96%. In certain embodiments, each coupling step independently has a stereoselectivity of at least 97%. In certain embodiments, each coupling step independently has a stereoselectivity of at least 98%. In certain embodiments, each coupling step independently has a stereoselectivity of at least 99%. In certain embodiments, each coupling step independently has a stereoselectivity of at least 99.5%. In certain embodiments, each coupling step independently has a stereoselectivity of virtually 100%. In certain embodiments, a coupling step has a stereoselectivity of virtually 100% in that each detectable product from the coupling step analyzed by an analytical method (e.g., NMR, HPLC, etc.) has the intended stereoselectivity. In certain embodiments, a chirally controlled internucleotidic linkage is typically formed with a stereoselectivity of at least 90%, 91%, 92%, 93%, 94%, 95%, 96%, 97%, 98%, 99.5% or virtually 100% (in certain embodiments, at least 90%; in certain embodiments, at least 95%; in certain embodiments, at least 96%; in certain embodiments, at least 97%; in certain embodiments, at least 98%; in certain embodiments, at least 99%). In certain embodiments, a chirally controlled internucleotidic linkage has a stereochemical purity (typically diastereomeric purity for oligonucleotides with multiple chiral centers) of at least 90%, 91%, 92%, 93%, 94%, 95%, 96%, 97%, 98%, 99.5% or virtually 100% (in certain embodiments, at least 90%; in certain embodiments, at least 95%; in certain embodiments, at least 96%; in certain embodiments, at least 97%; in certain embodiments, at least 98%; in certain embodiments, at least 99%) at its chiral linkage phosphorus. In certain embodiments, each chirally controlled internucleotidic linkage independently has a stereochemical purity (typically diastereomeric purity for oligonucleotides with multiple chiral centers) of at least 90%, 91%, 92%, 93%, 94%, 95%, 96%, 97%, 98%, 99.5% or virtually 100% (in certain embodiments, at least 90%; in certain embodiments, at least 95%; in certain embodiments, at least 96%; in certain embodiments, at least 97%; in certain embodiments, at least 98%; in certain embodiments, at least 99%) at its chiral linkage phosphorus. In certain embodiments, a non-chirally controlled internucleotidic linkage is typically formed with a stereoselectivity of less than 60%, 70%, 80%, 85%, or 90% (in certain embodiments, less than 60%; in certain embodiments, less than 70%; in certain embodiments, less than 80%; in certain embodiments, less than 85%; in certain embodiments, less than 90%). In certain embodiments, each non-chirally controlled internucleotidic linkage is independently formed with a stereoselectivity of less than 60%, 70%, 80%, 85%, or 90% (in certain embodiments, less than 60%; in certain embodiments, less than 70%; in certain embodiments, less than 80%; in certain embodiments, less than 85%; in certain embodiments, less than 90%). In certain embodiments, a non-chirally controlled internucleotidic linkage has a stereochemical purity (typically diastereomeric purity for oligonucleotides with multiple chiral centers) of less than 60%, 70%, 80%, 85%, or 90% (in certain embodiments, less than 60%; in certain embodiments, less than 70%; in certain embodiments, less than 80%; in certain embodiments, less than 85%; in certain embodiments, less than 90%) at its chiral linkage phosphorus. In certain embodiments, each non-chirally controlled internucleotidic linkage independently has a stereochemical purity (typically diastereomeric purity for oligonucleotides with multiple chiral centers) of less than 60%, 70%, 80%, 85%, or 90% (in certain embodiments, less than 60%; in certain embodiments, less than 70%; in certain embodiments, less than 80%; in certain embodiments, less than 85%; in certain embodiments, less than 90%) at its chiral linkage phosphorus.

In certain embodiments, at least 1, 2, 3, 4, 5, 6, 7, 8, 9, or 10 couplings of a monomer (as appreciated by those skilled in the art in certain embodiments a phosphoramidite for oligonucleotide synthesis) independently have a stereoselectivity less than about 60%, 70%, 80%, 85%, or 90% [for oligonucleotide synthesis, typically diastereoselectivity with respect to formed linkage phosphorus chiral center(s)]. In certain embodiments, at least one coupling has a stereoselectivity less than about 60%, 70%, 80%, 85%, or 90%. In certain embodiments, at least two couplings independently have a stereoselectivity less than about 60%, 70%, 80%, 85%, or 90%. In certain embodiments, at least three couplings independently have a stereoselectivity less than about 60%, 70%, 80%, 85%, or 90%. In certain embodiments, at least four couplings independently have a stereoselectivity less than about 60%, 70%, 80%, 85%, or 90%. In certain embodiments, at least five couplings independently have a stereoselectivity less than about 60%, 70%, 80%, 85%, or 90%. In certain embodiments, each coupling independently has a stereoselectivity less than about 60%, 70%, 80%, 85%, or 90%. In certain embodiments, each non-chirally controlled internucleotidic linkage is independently formed with a stereoselectivity less than about 60%, 70%, 80%, 85%, or 90%. In certain embodiments, a stereoselectivity is less than about 60%. In certain embodiments, a stereoselectivity is less than about 70%. In certain embodiments, a stereoselectivity is less than about 80%. In certain embodiments, a stereoselectivity is less than about 90%. In certain embodiments, at least 1, 2, 3, 4, 5, 6, 7, 8, 9, 10, 11, 12, 13, 14, 15, 16, 17, 18, 19, 20, 21, 22, 23, 24, or 25 couplings independently have a stereoselectivity less than about 90%. In certain embodiments, at least one coupling has a stereoselectivity less than about 90%. In certain embodiments, at least two couplings have a stereoselectivity less than about 90%. In certain embodiments, at least three couplings have a stereoselectivity less than about 90%. In certain embodiments, at least four couplings have a stereoselectivity less than about 90%. In certain embodiments, at least five couplings have a stereoselectivity less than about 90%. In certain embodiments, each coupling independently has a stereoselectivity less than about 90%. In certain embodiments, at least 1, 2, 3, 4, 5, 6, 7, 8, 9, 10, 11, 12, 13, 14, 15, 16, 17, 18, 19, 20, 21, 22, 23, 24, or 25 couplings independently have a stereoselectivity less than about 85%. In certain embodiments, each coupling independently has a stereoselectivity less than about 85%. In certain embodiments, at least 1, 2, 3, 4, 5, 6, 7, 8, 9, 10, 11, 12, 13, 14, 15, 16, 17, 18, 19, 20, 21, 22, 23, 24, or 25 couplings independently have a stereoselectivity less than about 80%. In certain embodiments, each coupling independently has a stereoselectivity less than about 80%. In certain embodiments, at least 1, 2, 3, 4, 5, 6, 7, 8, 9, 10, 11, 12, 13, 14, 15, 16, 17, 18, 19, 20, 21, 22, 23, 24, or 25 couplings independently have a stereoselectivity less than about 70%. In certain embodiments, each coupling independently has a stereoselectivity less than about 70%.

In certain embodiments, ds oligonucleotides and compositions of the present disclosure have high purity. In certain embodiments, ds oligonucleotides and compositions of the present disclosure have high stereochemical purity. In certain embodiments, a stereochemical purity, e.g., diastereomeric purity, is about 60%-100%. In certain embodiments, a diastereomeric purity, is about 60%-100%. In certain embodiments, the percentage is at least 60%, 65%, 70%, 75%, 80%, 85%, 90%, 91%, 92%, 93%, 93%, 95%, 96%, 97%, 98%, or 99%. In certain embodiments, the percentage is at least 80%, 85%, 90%, 91%, 92%, 93%, 93%, 95%, 96%, 97%, 98%, or 99%. In certain embodiments, the percentage is at least 90%, 91%, 92%, 93%, 93%, 95%, 96%, 97%, 98%, or 99%. In certain embodiments, a diastereomeric purity is at least 60%. In certain embodiments, a diastereomeric purity is at least 70%. In certain embodiments, a diastereomeric purity is at least 80%. In certain embodiments, a diastereomeric purity is at least 85%. In certain embodiments, a diastereomeric purity is at least 90%. In certain embodiments, a diastereomeric purity is at least 91%. In certain embodiments, a diastereomeric purity is at least 92%. In certain embodiments, a diastereomeric purity is at least 93%. In certain embodiments, a diastereomeric purity is at least 94%. In certain embodiments, a diastereomeric purity is at least 95%. In certain embodiments, a diastereomeric purity is at least 96%. In certain embodiments, a diastereomeric purity is at least 97%. In certain embodiments, a diastereomeric purity is at least 98%. In certain embodiments, a diastereomeric purity is at least 99%. In certain embodiments, a diastereomeric purity is at least 99.5%.

In certain embodiments, compounds of the present disclosure (e.g., oligonucleotides, chiral auxiliaries, etc.) comprise multiple chiral elements (e.g., multiple carbon and/or phosphorus (e.g., linkage phosphorus of chiral internucleotidic linkages) chiral centers). In certain embodiments, at least 1, 2, 3, 4, 5, 6, 7, 8, 9 or more chiral elements of a provided compound (e.g., a ds oligonucleotide) each independently have a diastereomeric purity as described herein. In certain embodiments, at least 1, 2, 3, 4, 5, 6, 7, 8, 9 or more chiral carbon centers of a provided compound each independently have a diastereomeric purity as described herein. In certain embodiments, at least 1, 2, 3, 4, 5, 6, 7, 8, 9 or more chiral phosphorus centers of a provided compound each independently have a diastereomeric purity as described herein. In certain embodiments, each chiral element independently has a diastereomeric purity as described herein. In certain embodiments, each chiral center independently has a diastereomeric purity as described herein. In certain embodiments, each chiral carbon center independently has a diastereomeric purity as described herein. In certain embodiments, each chiral phosphorus center independently has a diastereomeric purity as described herein. In certain embodiments, each chiral phosphorus center independently has a diastereomeric purity of at least 90%, 91%, 92%, 93%, 93%, 95%, 96%, 97%, 98%, or 99% or more.

As understood by a person having ordinary skill in the art, in certain embodiments, diastereoselectivity of a coupling or diastereomeric purity of a chiral linkage phosphorus center can be assessed through the diastereoselectivity of a dimer formation or diastereomeric purity of a dimer prepared under the same or comparable conditions, wherein the dimer has the same 5′- and 3′-nucleosides and internucleotidic linkage.

Various technologies can be utilized for identifying or confirming stereochemistry of chiral elements (e.g., configuration of chiral linkage phosphorus) and/or patterns of backbone chiral centers, and/or for assessing stereoselectivity (e.g., diastereoselectivity of couple steps in oligonucleotide synthesis) and/or stereochemical purity (e.g., diastereomeric purity of internucleotidic linkages, compounds (e.g., oligonucleotides), etc.). Example technologies include NMR [e.g., 1D (one-dimensional) and/or 2D (two-dimensional) ¹H-³¹P HETCOR (heteronuclear correlation spectroscopy)], HPLC, RP-HPLC, mass spectrometry, LC-MS, and cleavage of internucleotidic linkages by stereospecific nucleases, etc., which may be utilized individually or in combination. Example useful nucleases include benzonase, micrococcal nuclease, and svPDE (snake venom phosphodiesterase), which are specific for certain internucleotidic linkages with Rp linkage phosphorus (e.g., a Rp phosphorothioate linkage); and nuclease P1, mung bean nuclease, and nuclease Si, which are specific for internucleotidic linkages with Sp linkage phosphorus (e.g., a Sp phosphorothioate linkage). Without wishing to be bound by any particular theory, the present disclosure notes that, in at least some cases, cleavage of oligonucleotides by a particular nuclease may be impacted by structural elements, e.g., chemical modifications (e.g., 2′-modifications of a sugars), base sequences, or stereochemical contexts. For example, it is observed that in some cases, benzonase and micrococcal nuclease, which are specific for internucleotidic linkages with Rp linkage phosphorus, were unable to cleave an isolated Rp phosphorothioate internucleotidic linkage flanked by Sp phosphorothioate internucleotidic linkages.

In certain embodiments, ds oligonucleotides sharing a common base sequence, a common pattern of backbone linkages, and a common pattern of backbone chiral centers share a common pattern of backbone phosphorus modifications and a common pattern of base modifications. In certain embodiments, sd oligonucleotide compositions sharing a common base sequence, a common pattern of backbone linkages, and a common pattern of backbone chiral centers share a common pattern of backbone phosphorus modifications and a common pattern of nucleoside modifications. In certain embodiments, ds oligonucleotides share a common base sequence, a common pattern of backbone linkages, and a common pattern of backbone chiral centers have identical structures.

In certain embodiments, the present disclosure provides a ds oligonucleotide composition comprising a plurality of oligonucleotides capable of directing RNAi knockdown, wherein ds oligonucleotides of the plurality are of a particular ds oligonucleotide type, which composition is chirally controlled in that it is enriched, relative to a substantially racemic preparation of ds oligonucleotides having the same base sequence, for ds oligonucleotides of the particular ds oligonucleotide type.

In certain embodiments, ds oligonucleotides having a common base sequence, a common pattern of backbone linkages, and a common pattern of backbone chiral centers have a common pattern of backbone phosphorus modifications and a common pattern of base modifications. In certain embodiments, ds oligonucleotides having a common base sequence, a common pattern of backbone linkages, and a common pattern of backbone chiral centers have a common pattern of backbone phosphorus modifications and a common pattern of nucleoside modifications. In certain embodiments, ds oligonucleotides having a common base sequence, a common pattern of backbone linkages, and a common pattern of backbone chiral centers have identical structures.

In certain embodiments, the present disclosure provides dsRNAi oligonucleotide compositions comprising a plurality of oligonucleotides. In certain embodiments, the present disclosure provides chirally controlled oligonucleotide compositions of dsRNAi oligonucleotides. In certain embodiments, the present disclosure provides a dsRNAi oligonucleotide whose base sequence is or is complementary to a dsRNAi sequence disclosed herein or a portion thereof (e.g., various bases sequences in Table 1, wherein each T may be independently replaced with U and vice versa). In certain embodiments, the present disclosure provides a dsRNAi oligonucleotide whose base sequence comprises a base sequence that is or is complementary to a dsRNAi sequence disclosed herein or a portion thereof (e.g., various bases sequences in Table 1). In certain embodiments, the present disclosure provides a dsRNAi oligonucleotide whose base sequence comprises 15 contiguous bases of a base sequence that is or is complementary to a dsRNAi sequence disclosed herein or a portion thereof (e.g., various bases sequences in Table 1, wherein each T may be independently replaced with U and vice versa). In certain embodiments, the present disclosure provides a dsRNAi oligonucleotide which has a base sequence comprising 15 contiguous bases with 0-3 mismatches of a base sequence that is or is complementary to a dsRNAi sequence disclosed herein or a portion thereof (e.g., various bases sequences in Table 1, wherein each T may be independently replaced with U and vice versa). In certain embodiments, the present disclosure provides a dsRNAi oligonucleotide composition wherein the dsRNAi oligonucleotides comprise at least one chiral internucleotidic linkage which is not chirally controlled. In certain embodiments, the present disclosure provides a dsRNAi oligonucleotide comprising a non-chirally controlled chiral internucleotidic linkage, wherein the base sequence of the dsRNAi oligonucleotide comprises a base sequence that is or is complementary to a dsRNAi sequence disclosed herein or a portion thereof (e.g., various bases sequences in Table 1, wherein each T may be independently replaced with U and vice versa). In certain embodiments, the present disclosure provides a dsRNAi oligonucleotide composition comprising a non-chirally controlled chiral internucleotidic linkage, wherein the base sequence of the dsRNAi oligonucleotide is a base sequence that is or is complementary to a dsRNAi sequence disclosed herein or a portion thereof (e.g., various bases sequences in Table 1, wherein each T may be independently replaced with U and vice versa). In certain embodiments, the present disclosure provides a RNAi oligonucleotide comprising a non-chirally controlled chiral internucleotidic linkage, wherein the base sequence of the dsRNAi oligonucleotide comprises 15 contiguous bases of a base sequence that is or is complementary to a dsRNAi sequence disclosed herein or a portion thereof (e.g., various bases sequences in Table 1, wherein each T may be independently replaced with U and vice versa). In certain embodiments, the present disclosure provides a dsRNAi oligonucleotide comprising a non-chirally controlled chiral internucleotidic linkage, wherein the base sequence of the dsRNAi oligonucleotides comprises 15 contiguous bases with 0-3 mismatches of a base sequence that is or is complementary to a RNAi sequence disclosed herein or a portion thereof (e.g., various bases sequences in Table 1, wherein each T may be independently replaced with U and vice versa). In certain embodiments, the present disclosure provides a dsRNAi oligonucleotide comprising a chirally controlled chiral internucleotidic linkage, wherein the base sequence of the dsRNAi oligonucleotide comprises a base sequence that is or is complementary to a dsRNAi sequence disclosed herein or a portion thereof (e.g., various bases sequences in Table 1, wherein each T may be independently replaced with U and vice versa). In certain embodiments, the present disclosure provides a dsRNAi oligonucleotide composition comprising a chirally controlled chiral internucleotidic linkage, wherein the base sequence of the RNAi oligonucleotide is a base sequence that is or is complementary to a dsRNAi sequence disclosed herein or a portion thereof (e.g., various bases sequences in Table 1, wherein each T may be independently replaced with U and vice versa). In certain embodiments, the present disclosure provides a dsRNAi oligonucleotide comprising a chirally controlled chiral internucleotidic linkage, wherein the base sequence of the dsRNAi oligonucleotide comprises 15 contiguous bases of a base sequence that is or is complementary to a dsRNAi sequence disclosed herein or a portion thereof (e.g., various bases sequences in Table 1, wherein each T may be independently replaced with U and vice versa). In certain embodiments, the present disclosure provides a RNAi oligonucleotide comprising a chirally controlled chiral internucleotidic linkage, wherein the base sequence of the RNAi oligonucleotides comprises 15 contiguous bases with 0-3 mismatches of a base sequence that is or is complementary to a dsRNAi sequence disclosed herein or a portion thereof (e.g., various bases sequences in Table 1, wherein each T may be independently replaced with U and vice versa).

In certain embodiments, ds oligonucleotides of the same ds oligonucleotide type have a common pattern of backbone phosphorus modifications and a common pattern of nucleoside modifications. In certain embodiments, ds oligonucleotides of the same ds doligonucleotide type have a common pattern of sugar modifications. In certain embodiments, ds oligonucleotides of the same ds oligonucleotide type have a common pattern of base modifications. In certain embodiments, ds oligonucleotides of the same ds oligonucleotide type have a common pattern of nucleoside modifications. In certain embodiments, ds oligonucleotides of the same ds oligonucleotide type have the same constitution. In certain embodiments, ds oligonucleotides of the same ds oligonucleotide type are identical. In certain embodiments, ds oligonucleotides of the same ds oligonucleotide type are of the same ds oligonucleotide (as those skilled in the art will appreciate, such ds oligonucleotides may each independently exist in one of the various forms of the ds oligonucleotide, and may be the same, or different forms of the ds oligonucleotide). In certain embodiments, ds oligonucleotides of the same ds oligonucleotide type are each independently of the same ds oligonucleotide or a pharmaceutically acceptable salt thereof.

In certain embodiments, a plurality of ds oligonucleotides or ds oligonucleotides of a particular ds oligonucleotide type in a provided ds oligonucleotide composition are sdRNAi oligonucleotides. In certain embodiments, the present disclosure provides a chirally controlled dsRNAi oligonucleotide composition comprising a plurality of dsRNAi oligonucleotides, wherein the ds oligonucleotides share:
1) a common base sequence;
2) a common pattern of backbone linkages; and
3) the same linkage phosphorus stereochemistry at one or more chiral internucleotidic linkages (chirally controlled internucleotidic linkages), wherein the composition is enriched, relative to a substantially racemic preparation of oligonucleotides sharing the common base sequence and pattern of backbone linkages, for oligonucleotides of the plurality.

In certain embodiments, as used herein, “one or more” or “at least one” is 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, or more.

In certain embodiments, a ds oligonucleotide type is further defined by: 4) additional chemical moiety, if any.

In certain embodiments, the percentage is at least about 10%. In certain embodiments, the percentage is at least about 20%. In certain embodiments, the percentage is at least about 30%. In certain embodiments, the percentage is at least about 40%. In certain embodiments, the percentage is at least about 50%. In certain embodiments, the percentage is at least about 60%. In certain embodiments, the percentage is at least about 70%. In certain embodiments, the percentage is at least about 75%. In certain embodiments, the percentage is at least about 80%. In certain embodiments, the percentage is at least about 85%. In certain embodiments, the percentage is at least about 90%. In certain embodiments, the percentage is at least about 91%. In certain embodiments, the percentage is at least about 92%. In certain embodiments, the percentage is at least about 93%. In certain embodiments, the percentage is at least about 94%. In certain embodiments, the percentage is at least about 95%. In certain embodiments, the percentage is at least about 96%. In certain embodiments, the percentage is at least about 97%. In certain embodiments, the percentage is at least about 98%. In certain embodiments, the percentage is at least about 99%. In certain embodiments, the percentage is or is greater than (DS)^{nc}, wherein DS and nc are each independently as described in the present disclosure.

In certain embodiments, a plurality of ds oligonucleotides, e.g., dsRNAi oligonucleotides, share the same constitution. In certain embodiments, a plurality of oligonucleotides, e.g., dsRNAi oligonucleotides, are identical (the same stereoisomer). In certain embodiments, a chirally controlled ds oligonucleotide composition, e.g., a chirally controlled dsRNAi oligonucleotide composition, is a stereopure ds oligonucleotide composition wherein ds oligonucleotides of the plurality are identical (the same stereoisomer), and the composition does not contain any other stereoisomers. Those skilled in the art will appreciate that one or more other stereoisomers may exist as impurities as processes, selectivities, purifications, etc. may not achieve completeness.

In certain embodiments, a provided composition is characterized in that when it is contacted with a target nucleic acid (e.g., a transcript (e.g., pre-mRNA, mature mRNA, other types of RNA, etc. that hybridizes with oligonucleotides of the composition)), levels of the target nucleic acid and/or a product encoded thereby is reduced compared to that observed under a reference condition. In certain embodiments, a reference condition is selected from the group consisting of absence of the composition, presence of a reference composition, and combinations thereof. In certain embodiments, a reference condition is absence of the composition. In certain embodiments, a reference condition is presence of a reference composition. In certain embodiments, a reference composition is a composition whose oligonucleotides do not hybridize with the target nucleic acid. In certain embodiments, a reference composition is a composition whose oligonucleotides do not comprise a sequence that is sufficiently complementary to the target nucleic acid. In certain embodiments, a provided composition is a chirally controlled oligonucleotide composition and a reference composition is a non-chirally controlled oligonucleotide composition which is otherwise identical but is not chirally controlled (e.g., a racemic preparation of oligonucleotides of the same constitution as oligonucleotides of a plurality in the chirally controlled oligonucleotide composition).

In certain embodiments, the present disclosure provides a chirally controlled dsRNAi oligonucleotide composition comprising a plurality of dsRNAi oligonucleotides capable of directing RNAi knockdown, wherein the oligonucleotides share:
1) a common base sequence,
2) a common pattern of backbone linkages, and
3) the same linkage phosphorus stereochemistry at one or more (e.g., 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, or more) chiral internucleotidic linkages (chirally controlled internucleotidic linkages),
   wherein the composition is enriched, relative to a substantially racemic preparation of oligonucleotides sharing the common base sequence and pattern of backbone linkages, for oligonucleotides of the plurality, the ds oligonucleotide composition being characterized in that, when it is contacted with a transcript in a dsRNAi knockdown system, knockdown of the transcript is improved relative to that observed under reference conditions selected from the group consisting of absence of the composition, presence of a reference composition, and combinations thereof.

As noted above and understood in the art, in certain embodiments, the base sequence of a ds oligonucleotide may refer to the identity and/or modification status of nucleoside residues (e.g., of sugar and/or base components, relative to standard naturally occurring nucleotides such as adenine, cytosine, guanosine, thymine, and uracil) in the ds oligonucleotide and/or to the hybridization character (i.e., the ability to hybridize with particular complementary residues) of such residues.

As demonstrated herein, ds oligonucleotide structural elements (e.g., patterns of sugar modifications, backbone linkages, backbone chiral centers, backbone phosphorus modifications, etc.) and combinations thereof can provide surprisingly improved properties and/or bioactivities.

In certain embodiments, ds oligonucleotide compositions are capable of reducing the expression, level and/or activity of a target gene or a gene product thereof. In certain embodiments, ds oligonucleotide compositions are capable of reducing in the expression, level and/or activity of a target gene or a gene product thereof by sterically blocking translation after annealing to a target gene mRNA, by cleaving mRNA (pre-mRNA or mature mRNA), and/or by altering or interfering with mRNA splicing. In certain embodiments, provided dsRNAi oligonucleotide compositions are capable of reducing the expression, level and/or activity of a target gene or a gene product thereof. In certain embodiments, provided dsRNAi oligonucleotide compositions are capable of reducing in the expression, level and/or activity of a target gene or a gene product thereof by sterically blocking translation after annealing to a target gene mRNA, by cleaving target mRNA (pre-mRNA or mature mRNA), and/or by altering or interfering with mRNA splicing.

In certain embodiments, a ds oligonucleotide composition, e.g., a dsdRNAi oligonucleotide composition, is a substantially pure preparation of a single ds oligonucleotide stereoisomer, e.g., a dsRNAi oligonucleotide stereoisomer, in that oligonucleotides in the composition that are not of the oligonucleotide stereoisomer are impurities from the preparation process of said ds oligonucleotide stereoisomer, in some case, after certain purification procedures.

In certain embodiments, the present disclosure provides ds oligonucleotides and oligonucleotide compositions that are chirally controlled, and in certain embodiments, stereopure. For instance, in certain embodiments, a provided composition contains non-random or controlled levels of one or more individual oligonucleotide types as described herein. In certain embodiments, oligonucleotides of the same oligonucleotide type are identical.

### 3. Sugars

Various sugars, including modified sugars, can be utilized in accordance with the present disclosure. In certain embodiments, the present disclosure provides sugar modifications and patterns thereof optionally in combination with other structural elements (e.g., internucleotidic linkage modifications and patterns thereof, pattern of backbone chiral centers thereof, etc.) that when incorporated into oligonucleotides can provide improved properties and/or activities.

The most common naturally occurring nucleosides comprise ribose sugars (e.g., in RNA) or deoxyribose sugars (e.g., in DNA) linked to the nucleobases adenosine (A), cytosine (C), guanine (G), thymine (T) or uracil (U). In certain embodiments, a sugar, e.g., various sugars in many oligonucleotides in Table 1 (unless otherwise notes), is a natural DNA sugar (in DNA nucleic acids or oligonucleotides, having the structure of

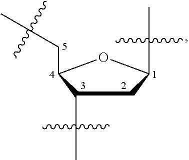 [SMILES: *C[C@H]1O[C@@H](*)C[C@H]1*]

wherein a nucleobase is attached to the 1′ position, and the 3′ and 5′ positions are connected to internucleotidic linkages (as appreciated by those skilled in the art, if at the 5′-end of a ds oligonucleotide, the 5′ position may be connected to a 5′-end group (e.g., —OH), and if at the 3′-end of a ds oligonucleotide, the 3′ position may be connected to a 3′-end group (e.g., —OH). In certain embodiments, a sugar is a natural RNA sugar (in RNA nucleic acids or oligonucleotides, having the structure of

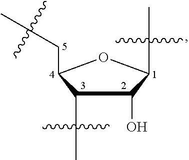

wherein a nucleobase is attached to the 1′ position, and the 3′ and 5′ positions are connected to internucleotidic linkages (as appreciated by those skilled in the art, if at the 5′-end of a ds oligonucleotide, the 5′ position may be connected to a 5′-end group (e.g., —OH), and if at the 3′-end of a ds oligonucleotide, the 3′ position may be connected to a 3′-end group (e.g., —OH). In certain embodiments, a sugar is a modified sugar in that it is not a natural DNA sugar or a natural RNA sugar. Among other things, modified sugars may provide improved stability. In certain embodiments, modified sugars can be utilized to alter and/or optimize one or more hybridization characteristics. In certain embodiments, modified sugars can be utilized to alter and/or optimize target recognition. In certain embodiments, modified sugars can be utilized to optimize Tm. In certain embodiments, modified sugars can be utilized to improve oligonucleotide activities.

Sugars can be bonded to internucleotidic linkages at various positions. As non-limiting examples, internucleotidic linkages can be bonded to the 2′, 3′, 4′ or 5′ positions of sugars. In certain embodiments, as most commonly in natural nucleic acids, an internucleotidic linkage connects with one sugar at the 5′ position and another sugar at the 3′ position unless otherwise indicated.

In certain embodiments, a sugar is an optionally substituted natural DNA or RNA sugar. In certain embodiments, a sugar is optionally substituted

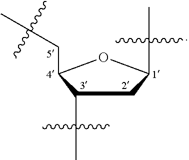 [SMILES: *C[C@H]1O[C@@H](*)C[C@H]1*]

In certain embodiments, the 2′ position is optionally substituted. In certain embodiments, a sugar is

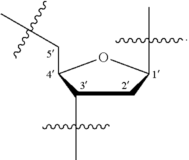 [SMILES: *C[C@H]1O[C@@H](*)C[C@H]1*]

In certain embodiments, a sugar has the structure of

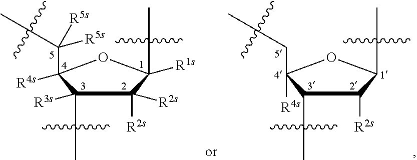

wherein each of R^{1s}, R^{2s}, R^{3s}, R^{4s}, and R^{5s} is independently —H, a suitable substituent or suitable sugar modification (e.g., those described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019/032612, WO 2019/055951, and/or WO 2019/075357, the substituents, sugar modifications, descriptions of R^{1s}, R^{2s}, R^{3s}, R^{4s}, and R^{5s}, and modified sugars of each of which are independently incorporated herein by reference). In certain embodiments, a sugar has the structure of

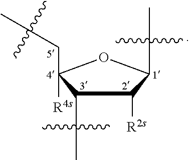

In certain embodiments, R^{4s} is —H. In certain embodiments, a sugar has the structure of

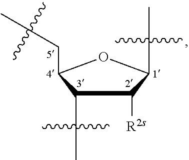

wherein R^{2s} is —H, halogen, or —OR, wherein R is optionally substituted C₁₋₆ aliphatic. In certain embodiments, R^{2s} is —H. In certain embodiments, R^{2s} is —F. In certain embodiments, R^{2s} is —OMe. In certain embodiments, R^{2s} is —OCH₂CH₂OMe.

In certain embodiments, a sugar has the structure of

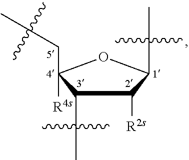

wherein R^{2s} and R^{4s} are taken together to form -L^{s}-, wherein L^{s} is a covalent bond or optionally substituted bivalent C₁₋₆ aliphatic or heteroaliphatic having 1-4 heteroatoms. In certain embodiments, each heteroatom is independently selected from nitrogen, oxygen or sulfur). In certain embodiments, L^{s} is optionally substituted C2-O—CH₂-C4. In certain embodiments, L^{s} is C2O—CH₂-C4. In certain embodiments, L^{s} is C2-O—(R)—CH(CH₂CH₃)-C4. In certain embodiments, L^{s} is C2-O—(S)—CH(CH₂CH₃)-C4.

In certain embodiments, a modified sugar contains one or more substituents at the 2′ position (typically one substituent, and often at the axial position) independently selected from —F; —CF₃, —CN, —N₃, —NO, —NO₂, —OR′, —SR′, or —N(R′)₂, wherein each R′ is independently optionally substituted C₁₋₁₀ aliphatic; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁0 alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—, —(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), wherein each of the alkyl, alkylene, alkenyl and alkynyl is independently and optionally substituted. In certain embodiments, a substituent is —O(CH₂)ₙOCH₃, —O(CH₂)ₙNH₂, MOE, DMAOE, or DMAEOE, wherein n is from 1 to about 10.

In certain embodiments, the 2′-OH of a ribose is replaced with a group selected from —H, —F; —CF₃, —CN, —N₃, —NO, —NO₂, —OR′, —SR′, or —N(R′)₂, wherein each R′ is independently described in the present disclosure; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—, —(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), wherein each of the alkyl, alkylene, alkenyl and alkynyl is independently and optionally substituted. In certain embodiments, the 2′-OH is replaced with —H (deoxyribose). In certain embodiments, the 2′-OH is replaced with —F. In certain embodiments, the 2′-OH is replaced with —OR′. In certain embodiments, the 2′-OH is replaced with —OMe. In certain embodiments, the 2′-OH is replaced with —OCH₂CH₂OMe.

In certain embodiments, a sugar modification is a 2′-modification. Commonly used 2′-modifications include but are not limited to 2′-OR, wherein R is optionally substituted C₁₋₆ aliphatic. In certain embodiments, a modification is 2′-OR, wherein R is optionally substituted C₁₋₆ alkyl. In certain embodiments, a modification is 2′-OMe. In certain embodiments, a modification is 2′-MOE. In certain embodiments, a 2′-modification is S-cEt. In certain embodiments, a modified sugar is an LNA sugar. In certain embodiments, a 2′-modification is —F.

In certain embodiments, a sugar modification replaces a sugar moiety with another cyclic or acyclic moiety. Examples of such moieties are widely known in the art, including but not limited to those used in morpholino (optionally with its phosphorodiamidate linkage), glycol nucleic acids, etc.

In certain embodiments, one or more of the sugars of an ATXN3 oligonucleotide are modified. In certain embodiments, each sugar of a ds oligonucleotide is independently modified. In certain embodiments, a modified sugar comprises a 2′-modification. In certain embodiments, each modified sugar independently comprises a 2′-modification. In certain embodiments, a 2′-modification is 2′-OR, wherein R is optionally substituted C₁₋₆ aliphatic. In certain embodiments, a 2′-modification is a 2′-OMe. In certain embodiments, a 2′-modification is a 2′-MOE. In certain embodiments, a 2′-modification is an LNA sugar modification. In certain embodiments, a 2′-modification is 2′-F. In certain embodiments, each sugar modification is independently a 2′-modification. In certain embodiments, each sugar modification is independently 2′-OR. In certain embodiments, each sugar modification is independently 2′-OR, wherein R is optionally substituted C₁₋₆ alkyl. In certain embodiments, each sugar modification is 2′-OMe. In certain embodiments, each sugar modification is 2′-MOE. In certain embodiments, each sugar modification is independently 2′-OMe or 2′-MOE. In certain embodiments, each sugar modification is independently 2′-OMe, 2′-MOE, or a LNA sugar.

As those skilled in the art will appreciate, modifications of sugars, nucleobases, internucleotidic linkages, etc. can and are often utilized in combination in oligonucleotides, e.g., see various oligonucleotides in Table 1. For example, a combination of sugar modification and nucleobase modification is 2′-F (sugar) 5-methyl (nucleobase) modified nucleosides. In certain embodiments, a combination is replacement of a ribosyl ring oxygen atom with S and substitution at the 2′-position.

In certain embodiments, a sugar is one described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612, the sugars of each of which is incorporated herein by reference.

Various additional sugars useful for preparing oligonucleotides or analogs thereof are known in the art and may be utilized in accordance with the present disclosure.

### 4. Nucleobases

Various nucleobases may be utilized in provided ds oligonucleotides in accordance with the present disclosure. In certain embodiments, a nucleobase is a natural nucleobase, the most commonly occurring ones being A, T, C, G and U. In certain embodiments, a nucleobase is a modified nucleobase in that it is not A, T, C, G or U. In certain embodiments, a nucleobase is optionally substituted A, T, C, G or U, or a substituted tautomer of A T, C, G or U. In certain embodiments, a nucleobase is optionally substituted A, T, C, G or U, e.g., 5mC, 5-hydroxymethyl C, etc. In certain embodiments, a nucleobase is alkyl-substituted A, T, C, G or U. In certain embodiments, a nucleobase is A. In certain embodiments, a nucleobase is T. In certain embodiments, a nucleobase is C. In certain embodiments, a nucleobase is G. In certain embodiments, a nucleobase is U. In certain embodiments, a nucleobase is 5mC. In certain embodiments, a nucleobase is substituted A, T, C, G or U. In certain embodiments, a nucleobase is a substituted tautomer of A, T, C, G or U. In certain embodiments, substitution protects certain functional groups in nucleobases to minimize undesired reactions during oligonucleotide synthesis. Suitable technologies for nucleobase protection in oligonucleotide synthesis are widely known in the art and may be utilized in accordance with the present disclosure. In certain embodiments, modified nucleobases improves properties and/or activities of ds oligonucleotides. For example, in many cases, 5mC may be utilized in place of C to modulate certain undesired biological effects, e.g., immune responses. In certain embodiments, when determining sequence identity, a substituted nucleobase having the same hydrogen-bonding pattern is treated as the same as the unsubstituted nucleobase, e.g., 5mC may be treated the same as C [e.g., a ds oligonucleotide having 5mC in place of C (e.g., AT5mCG) is considered to have the same base sequence as a ds oligonucleotide having C at the corresponding location(s) (e.g., ATCG)].

In certain embodiments, a ds oligonucleotide comprises one or more A, T, C, G or U. In certain embodiments, a ds oligonucleotide comprises one or more optionally substituted A, T, C, G or U. In certain embodiments, a ds oligonucleotide comprises one or more 5-methylcytidine, 5-hydroxymethylcytidine, 5-formylcytosine, or 5-carboxylcytosine. In certain embodiments, a ds oligonucleotide comprises one or more 5-methylcytidine. In certain embodiments, each nucleobase in a ds oligonucleotide is selected from the group consisting of optionally substituted A, T, C, G and U, and optionally substituted tautomers of A, T, C, G and U.

In certain embodiments, each nucleobase in a ds oligonucleotide is optionally protected A, T, C, G and U. In certain embodiments, each nucleobase in a ds oligonucleotide is optionally substituted A, T, C, G or U. In certain embodiments, each nucleobase in a ds oligonucleotide is selected from the group consisting of A, T, C, G, U, and 5mC.

In certain embodiments, a nucleobase is optionally substituted 2AP or DAP. In certain embodiments, a nucleobase is optionally substituted 2AP. In certain embodiments, a nucleobase is optionally substituted DAP. In certain embodiments, a nucleobase is 2AP. In certain embodiments, a nucleobase is DAP.

In certain embodiments, a nucleobase is a natural nucleobase or a modified nucleobase derived from a natural nucleobase. Examples include uracil, thymine, adenine, cytosine, and guanine optionally having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products). Certain examples of modified nucleobases are disclosed in Chiu and Rana, R N A, 2003, 9, 1034-1048, Limbach et al. Nucleic Acids Research, 1994, 22, 2183-2196 and Revankar and Rao, Comprehensive Natural Products Chemistry, vol. 7, 313. In certain embodiments, a modified nucleobase is substituted uracil, thymine, adenine, cytosine, or guanine. In certain embodiments, a modified nucleobase is a functional replacement, e.g., in terms of hydrogen bonding and/or base pairing, of uracil, thymine, adenine, cytosine, or guanine. In certain embodiments, a nucleobase is optionally substituted uracil, thymine, adenine, cytosine, 5-methylcytosine, or guanine. In certain embodiments, a nucleobase is uracil, thymine, adenine, cytosine, 5-methylcytosine, or guanine.

In certain embodiments, a provided ds oligonucleotide comprises one or more 5-methylcytosine. In certain embodiments, the present disclosure provides a ds oligonucleotide whose base sequence is disclosed herein, e.g., in Table 1, wherein each T may be independently replaced with U and vice versa, and each cytosine is optionally and independently replaced with 5-methylcytosine or vice versa. As appreciated by those skilled in the art, in certain embodiments, 5mC may be treated as C with respect to base sequence of an oligonucleotide—such oligonucleotide comprises a nucleobase modification at the C position (e.g., see various oligonucleotides in Table 1). In description of oligonucleotides, typically unless otherwise noted, nucleobases, sugars and internucleotidic linkages are non-modified.

In certain embodiments, a modified base is optionally substituted adenine, cytosine, guanine, thymine, or uracil, or a tautomer thereof. In certain embodiments, a modified nucleobase is a modified adenine, cytosine, guanine, thymine or uracil, modified by one or more modifications by which:
1) a nucleobase is modified by one or more optionally substituted groups independently selected from acyl, halogen, amino, azide, alkyl, alkenyl, alkynyl, aryl, heteroalkyl, heteroalkenyl, heteroalkynyl, heterocyclyl, heteroaryl, carboxyl, hydroxyl, biotin, avidin, streptavidin, substituted silyl, and combinations thereof;
2) one or more atoms of a nucleobase are independently replaced with a different atom selected from carbon, nitrogen and sulfur;
3) one or more double bonds in a nucleobase are independently hydrogenated; or
4) one or more aryl or heteroaryl rings are independently inserted into a nucleobase.

In certain embodiments, a modified nucleobase is a modified nucleobase known in the art, e.g., WO2017/210647. In certain embodiments, modified nucleobases are expanded-size nucleobases in which one or more aryl and/or heteroaryl rings, such as phenyl rings, have been added.

In certain embodiments, a modified nucleobase is selected from 5-substituted pyrimidines, 6-azapyrimidines, alkyl or alkynyl substituted pyrimidines, alkyl substituted purines, and N−2, N−6 and 0-6 substituted purines. In certain embodiments, modified nucleobases are selected from 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C—C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. In certain embodiments, modified nucleobases are tricyclic pyrimidines, such as 1,3-diazaphenoxazine-2-one, 1,3-diazaphenothiazine-2-one or 9-(2-aminoethoxy)-1,3-diazaphenoxazine-2-one (G-clamp). In certain embodiments, modified nucleobases are those in which the purine or pyrimidine base is replaced with other heterocycles, for example, 7-deaza-adenine, 7-deazaguanosine, 2-aminopyridine or 2-pyridone.

In certain embodiments, a modified nucleobase is substituted. In certain embodiments, a modified nucleobase is substituted such that it contains, e.g., heteroatoms, alkyl groups, or linking moieties connected to fluorescent moieties, biotin or avidin moieties, or other protein or peptides. In certain embodiments, a modified nucleobase is a “universal base” that is not a nucleobase in the most classical sense, but that functions similarly to a nucleobase. One example of a universal base is 3-nitropyrrole.

In certain embodiments, nucleosides that can be utilized in provided technologies comprise modified nucleobases and/or modified sugars, e.g., 4-acetylcytidine; 5-(carboxyhydroxylmethyl)uridine; 2′-O-methylcytidine; 5-carboxymethylaminomethyl-2-thiouridine; 5-carboxymethylaminomethyluridine; dihydrouridine; 2′-O-methylpseudouridine; beta,D-galactosylqueosine; 2′-O-methylguanosine; N-isopentenyladenosine; 1-methyladenosine; 1-methylpseudouridine; 1-methylguanosine; 1-methylinosine; 2,2-dimethylguanosine; 2-methyladenosine; 2-methylguanosine; N⁷-methylguanosine; 3-methyl-cytidine; 5-methylcytidine; 5-hydroxymethylcytidine; 5-formylcytosine; 5-carboxylcytosine; N⁶-methyladenosine; 7-methylguanosine; 5-methylaminoethyluridine; 5-methoxyaminomethyl-2-thiouridine; beta,D-mannosylqueosine; 5-methoxycarbonylmethyluridine; 5-methoxyuridine; 2-methylthio-N⁶-isopentenyladenosine; N-((9-beta,D-ribofuranosyl-2-methylthiopurine-6-yl)carbamoyl)threonine; N-((9-beta,D-ribofuranosylpurine-6-yl)-N-methylcarbamoyl)threonine; uridine-5-oxyacetic acid methylester; uridine-5-oxyacetic acid (v); pseudouridine; queosine; 2-thiocytidine; 5-methyl-2-thiouridine; 2-thiouridine; 4-thiouridine; 5-methyluridine; 2′-O-methyl-5-methyluridine; and 2′-O-methyluridine. In certain embodiments, a nucleobase, e.g., a modified nucleobase comprises one or more biomolecule binding moieties such as e.g., antibodies, antibody fragments, biotin, avidin, streptavidin, receptor ligands, or chelating moieties. In other embodiments, a nucleobase is 5-bromouracil, 5 iodouracil, or 2,6-diaminopurine. In certain embodiments, a nucleobase comprises substitution with a fluorescent or biomolecule binding moiety. In certain embodiments, a substituent is a fluorescent moiety.

In certain embodiments, a substituent is biotin or avidin.

In certain embodiments, a nucleobase is one described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612, the nucleobases of each of which is incorporated herein by reference.

### 5. Additional Chemical Moieties

In certain embodiments, a ds oligonucleotide comprises one or more additional chemical moieties. Various additional chemical moieties, e.g., targeting moieties, carbohydrate moieties, lipid moieties, etc. are known in the art and can be utilized in accordance with the present disclosure to modulate properties and/or activities of provided oligonucleotides, e.g., stability, half-life, activities, delivery, pharmacodynamics properties, pharmacokinetic properties, etc. In certain embodiments, certain additional chemical moieties facilitate delivery of oligonucleotides to desired cells, tissues and/or organs, including but not limited the cells of the central nervous system. In certain embodiments, certain additional chemical moieties facilitate internalization of oligonucleotides. In certain embodiments, certain additional chemical moieties increase oligonucleotide stability. In certain embodiments, the present disclosure provides technologies for incorporating various additional chemical moieties into oligonucleotides.

In certain embodiments, a ds oligonucleotide comprises an additional chemical moiety demonstrates increased delivery to and/or activity in a tissue compared to a reference oligonucleotide, e.g., a reference oligonucleotide which does not have the additional chemical moiety but is otherwise identical.

In certain embodiments, non-limiting examples of additional chemical moieties include carbohydrate moieties, targeting moieties, etc., which, when incorporated into oligonucleotides, can improve one or more properties. In certain embodiments, an additional chemical moiety is selected from: glucose, GluNAc (N-acetyl amine glucosamine) and anisamide moieties. In certain embodiments, a provided ds oligonucleotide can comprise two or more additional chemical moieties, wherein the additional chemical moieties are identical or non-identical, or are of the same category (e.g., carbohydrate moiety, sugar moiety, targeting moiety, etc.) or not of the same category.

In certain embodiments, an additional chemical moiety is a targeting moiety. In certain embodiments, an additional chemical moiety is or comprises a carbohydrate moiety. In certain embodiments, an additional chemical moiety is or comprises a lipid moiety. In certain embodiments, an additional chemical moiety is or comprises a ligand moiety for, e.g., cell receptors such as a sigma receptor, an asialoglycoprotein receptor, etc. In certain embodiments, a ligand moiety is or comprises an anisamide moiety, which may be a ligand moiety for a sigma receptor. In certain embodiments, a ligand moiety is or comprises a GalNAc moiety, which may be a ligand moiety for an asialoglycoprotein receptor. In certain embodiments, an additional chemical moiety facilitates delivery to liver.

In certain embodiments, a provided ds oligonucleotide can comprise one or more linkers and additional chemical moieties (e.g., targeting moieties), and/or can be chirally controlled or not chirally controlled, and/or have a bases sequence and/or one or more modifications and/or formats as described herein.

Various linkers, carbohydrate moieties and targeting moieties, including many known in the art, can be utilized in accordance with the present disclosure. In certain embodiments, a carbohydrate moiety is a targeting moiety. In certain embodiments, a targeting moiety is a carbohydrate moiety.

In certain embodiments, a provided ds oligonucleotide comprises an additional chemical moiety suitable for delivery, e.g., glucose, GluNAc (N-acetyl amine glucosamine), anisamide, or a structure selected from:

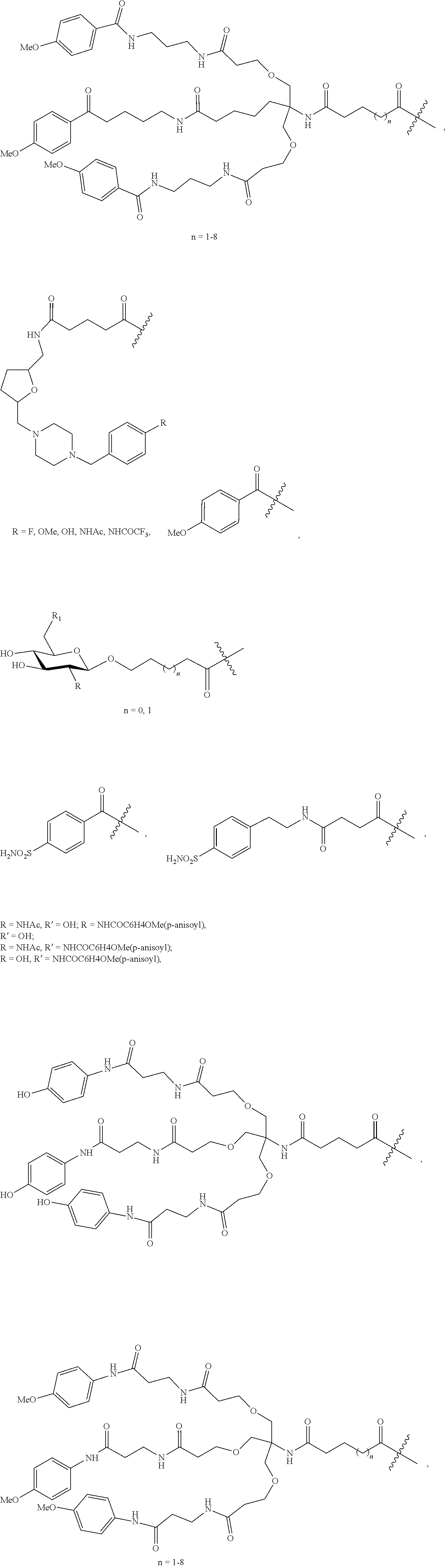

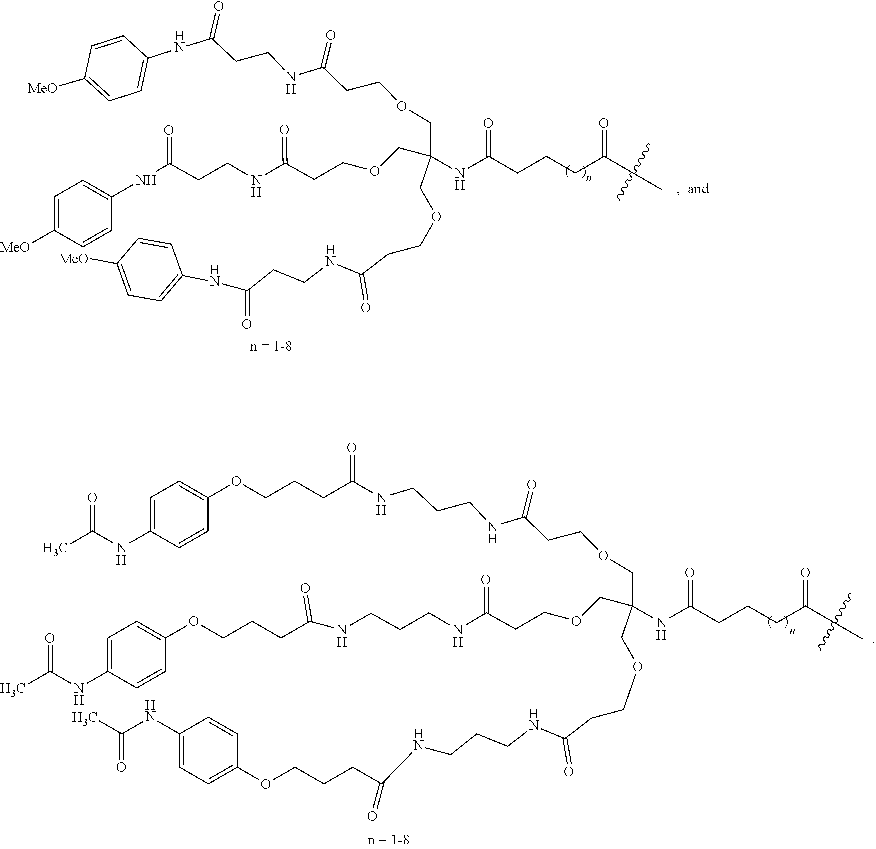

In certain embodiments, n is 1. In certain embodiments, n is 2. In certain embodiments, n is 3. In certain embodiments, n is 4. In certain embodiments, n is 5. In certain embodiments, n is 6. In certain embodiments, n is 7. In certain embodiments, n is 8.

In certain embodiments, additional chemical moieties are any of ones described in the Examples, including examples of various additional chemical moieties incorporated into various ds oligonucleotides.

In certain embodiments, an additional chemical moiety conjugated to a ds oligonucleotide is capable of targeting the ds oligonucleotide to a cell in the central nervous system.

In certain embodiments, an additional chemical moiety comprises or is a cell receptor ligand. In certain embodiments, an additional chemical moiety comprises or is a protein binder, e.g., one binds to a cell surface protein. Such moieties among other things can be useful for targeted delivery of ds oligonucleotides to cells expressing the corresponding receptors or proteins. In certain embodiments, an additional chemical moiety of a provided ds oligonucleotide comprises anisamide or a derivative or an analog thereof and is capable of targeting the ds oligonucleotide to a cell expressing a particular receptor, such as the sigma 1 receptor.

In certain embodiments, a provided ds oligonucleotide is formulated for administration to a body cell and/or tissue expressing its target. In certain embodiments, an additional chemical moiety conjugated to a ds oligonucleotide is capable of targeting the oligonucleotide to a cell.

In certain embodiments, an additional chemical moiety is selected from optionally substituted phenyl,

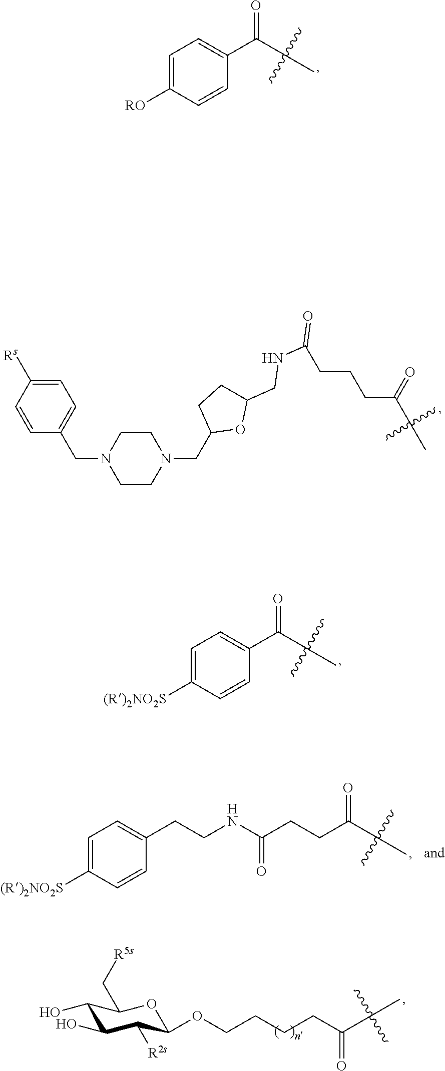

wherein n′ is 1, 2, 3, 4, 5, 6, 7, 8, 9, or 10, and each other variable is as described in the present disclosure. In certain embodiments, R^{s} is F. In certain embodiments, R^{s} is OMe. In certain embodiments, R^{s} is OH. In certain embodiments, R^{s} is NHAc. In certain embodiments, R^{s} is NHCOCF₃. In certain embodiments, R′ is H. In certain embodiments, R is H. In certain embodiments, R^{2s} is NHAc, and R^{5s} is OH. In certain embodiments, R^{2s} is p-anisoyl, and R^{5s} is OH. In certain embodiments, R^{2s} is NHAc and R^{5s} is p-anisoyl. In certain embodiments, R^{2s} is OH, and R^{5s} is p-anisoyl. In certain embodiments, an additional chemical moiety is selected from

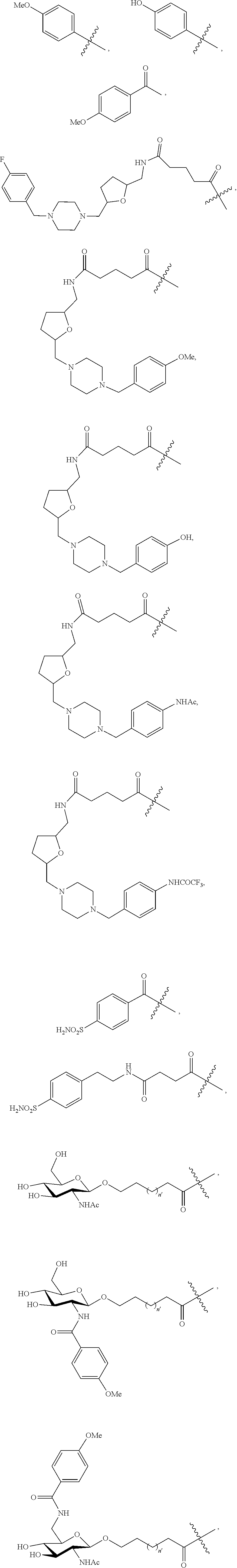

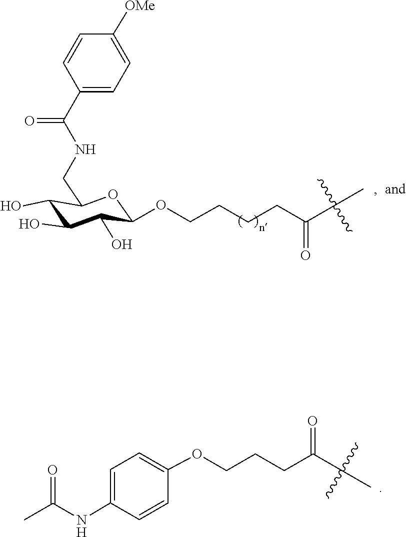

In certain embodiments, n′ is 1. In certain embodiments, n′ is 0. In certain embodiments, n″ is 1. In certain embodiments, n″ is 2.

In certain embodiments, an additional chemical moiety is or comprises an asialoglycoprotein receptor (ASGPR) ligand.

Without wishing to be bound by any particular theory, the present disclosure notes that ASGPR1 has also been reported to be expressed in the hippocampus region and/or cerebellum Purkinje cell layer of the mouse. http://mouse.brain-map.org/experiment/show/2048

Various other ASGPR ligands are known in the art and can be utilized in accordance with the present disclosure. In certain embodiments, an ASGPR ligand is a carbohydrate. In certain embodiments, an ASGPR ligand is GalNac or a derivative or an analog thereof. In certain embodiments, an ASGPR ligand is one described in Sanhueza et al. J. Am. Chem. Soc., 2017, 139 (9), pp 3528-3536. In certain embodiments, an ASGPR ligand is one described in Mamidyala et al. J. Am. Chem. Soc., 2012, 134, pp 1978-1981. In certain embodiments, an ASGPR ligand is one described in US 20160207953. In certain embodiments, an ASGPR ligand is a substituted-6,8-dioxabicyclo[3.2.1]octane-2,3-diol derivative disclosed in, e.g., US 20160207953. In certain embodiments, an ASGPR ligand is one described in, e.g., US 20150329555. In certain embodiments, an ASGPR ligand is a substituted-6,8-dioxabicyclo[3.2.1]octane-2,3-diol derivative disclosed e.g., in US 20150329555. In certain embodiments, an ASGPR ligand is one described in U.S. Pat. No. 8,877,917, US 20160376585, U.S. Ser. No. 10/086,081, or U.S. Pat. No. 8,106,022. ASGPR ligands described in these documents are incorporated herein by reference. Those skilled in the art will appreciate that various technologies are known in the art, including those described in these documents, for assessing binding of a chemical moiety to ASGPR and can be utilized in accordance with the present disclosure. In certain embodiments, a provided ds oligonucleotide is conjugated to an ASGPR ligand. In certain embodiments, a provided ds oligonucleotide comprises an ASGPR ligand. In certain embodiments, an additional chemical moiety comprises an ASGPR ligand is

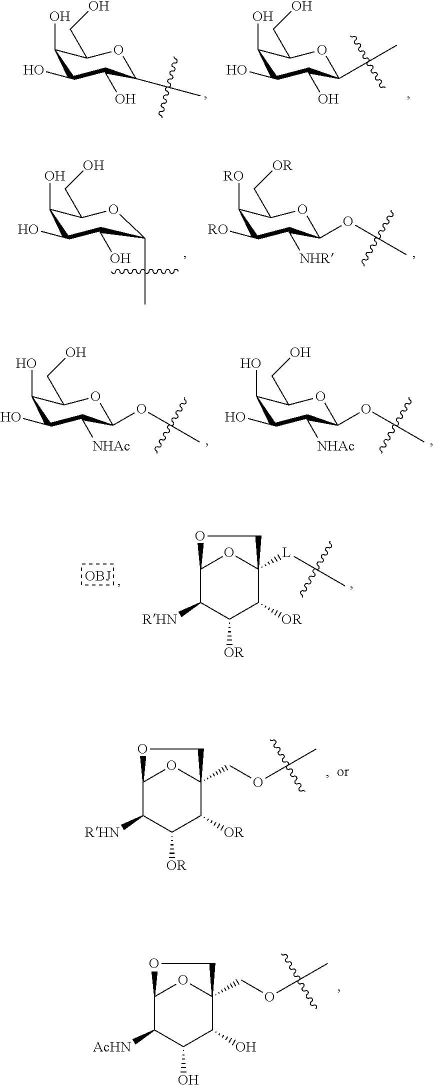

wherein each variable is independently as described in the present disclosure. In certain embodiments, R is —H. In certain embodiments, R′ is —C(O)R.

In certain embodiments, an additional chemical moiety is or comprises

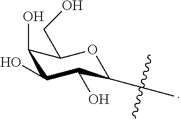

In certain embodiments, an additional chemical moiety is or comprises

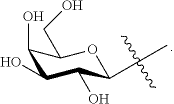

In certain embodiments, an additional chemical moiety is or comprises

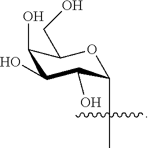

In certain embodiments, an additional chemical moiety is or comprises

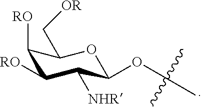

In certain embodiments, an additional chemical moiety is or comprises optionally substituted

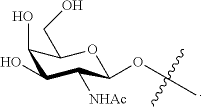

In certain embodiments, an additional chemical moiety is or comprises

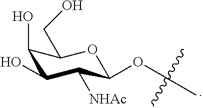

In certain embodiments, an additional chemical moiety is or comprises

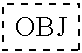

In certain embodiments, an additional chemical moiety is or comprises

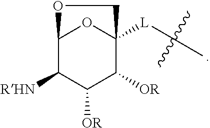

In certain embodiments, an additional chemical moiety is or comprises

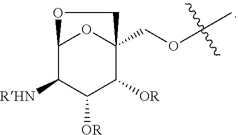

In certain embodiments, an additional chemical moiety is or comprises

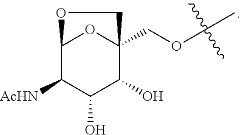 [SMILES: *OC[C@@]12CO[C@@H](O1)[C@H](NC(C)=O)[C@@H](O)[C@H]2O]

In certain embodiments, an additional chemical moiety comprises one or more moieties that can bind to, e.g., oligonucleotide target cells. For example, in certain embodiments, an additional chemistry moiety comprises one or more protein ligand moieties, e.g., in certain embodiments, an additional chemical moiety comprises multiple moieties, each of which independently is an ASGPR ligand. In certain embodiments, as in Mod 001, Mod083, Mod071, Mod153, and Mod155, an additional chemical moiety comprises three such ligands.

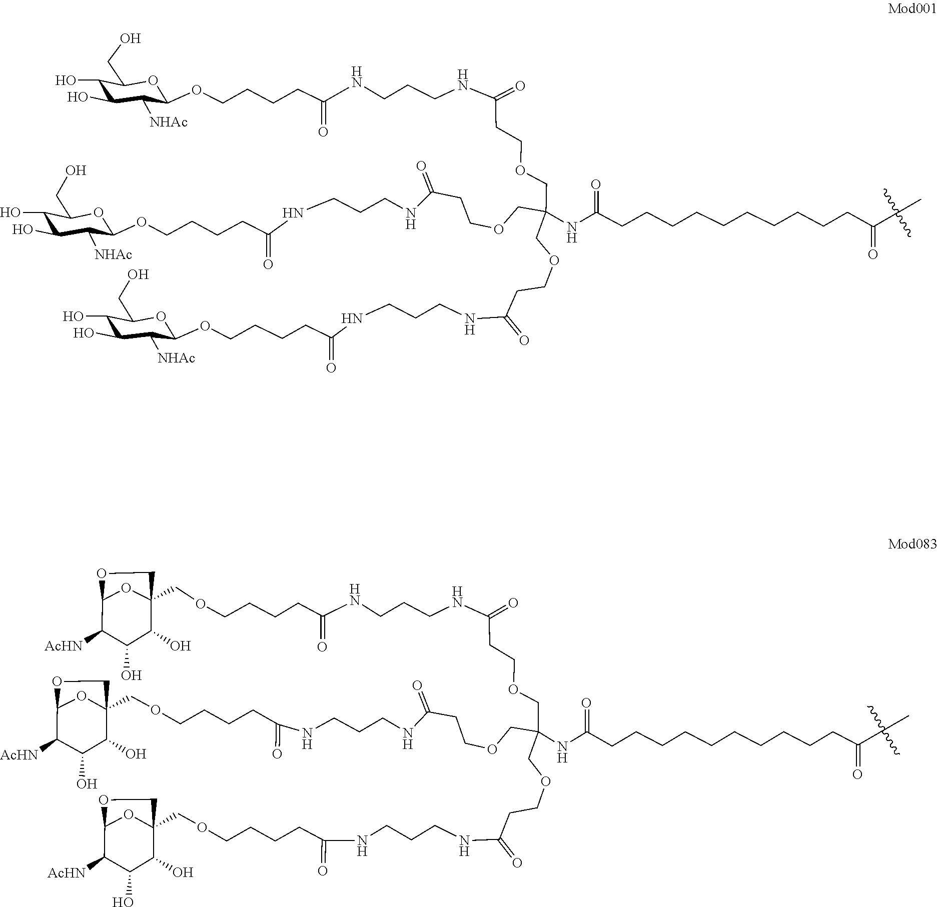

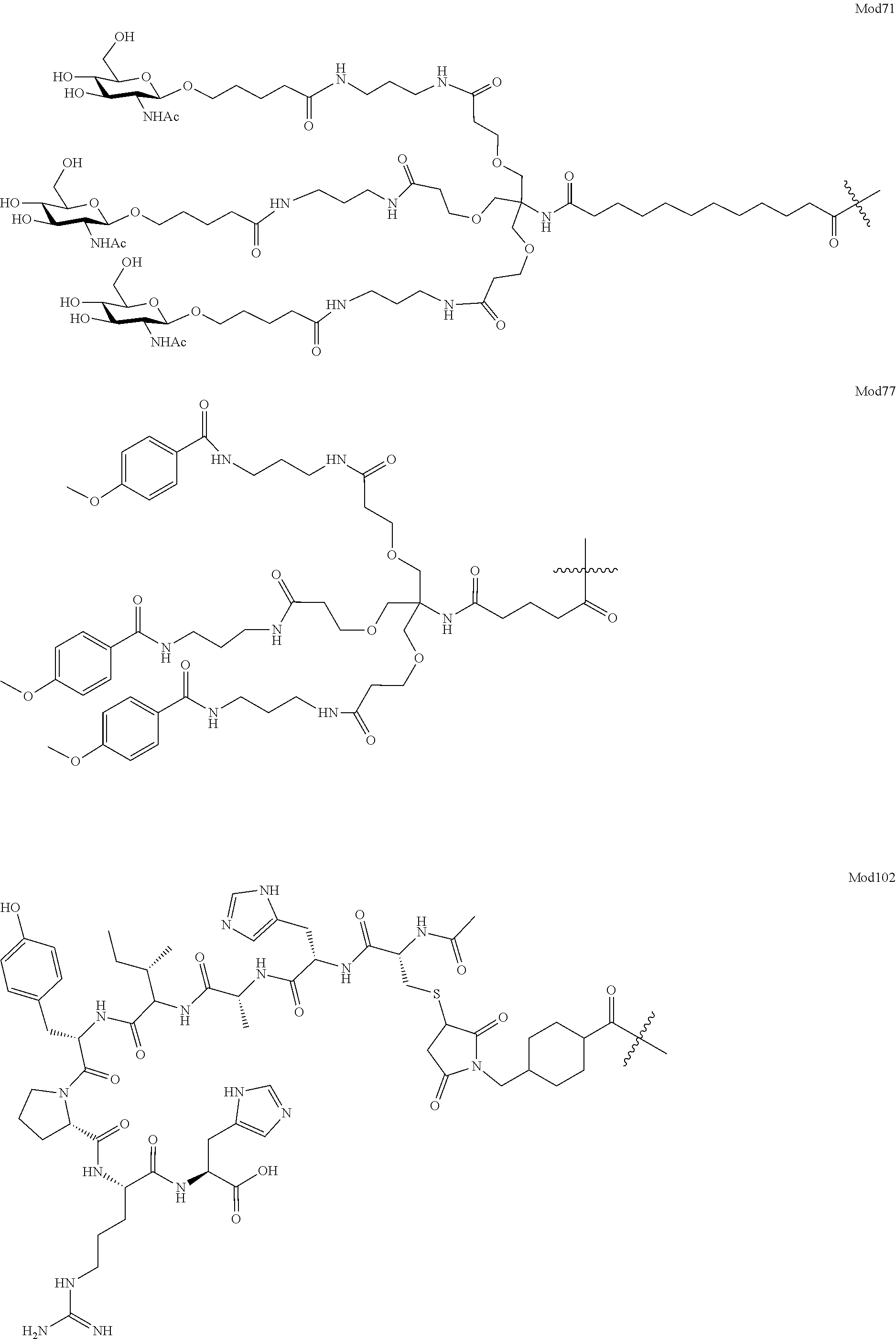

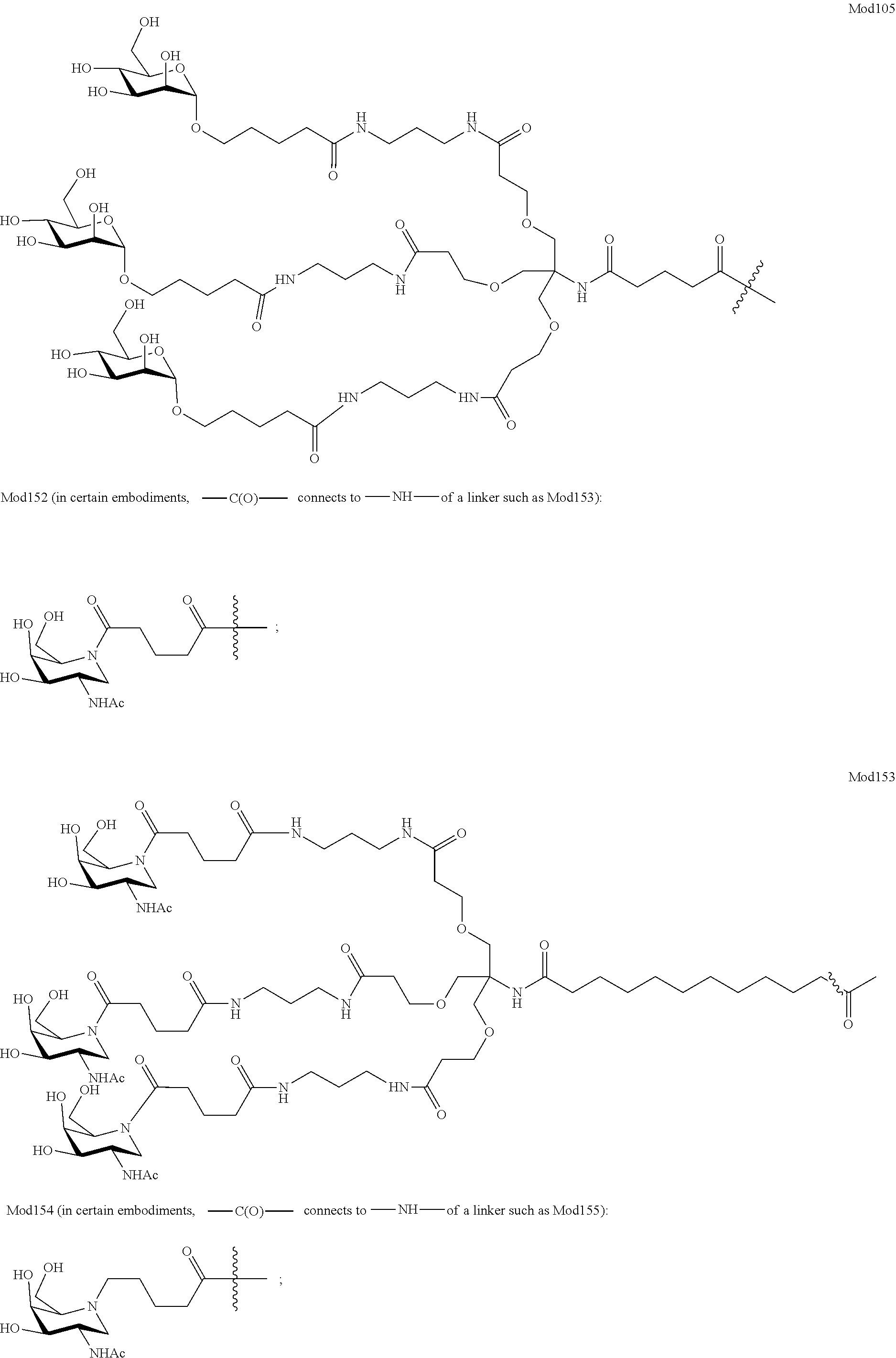

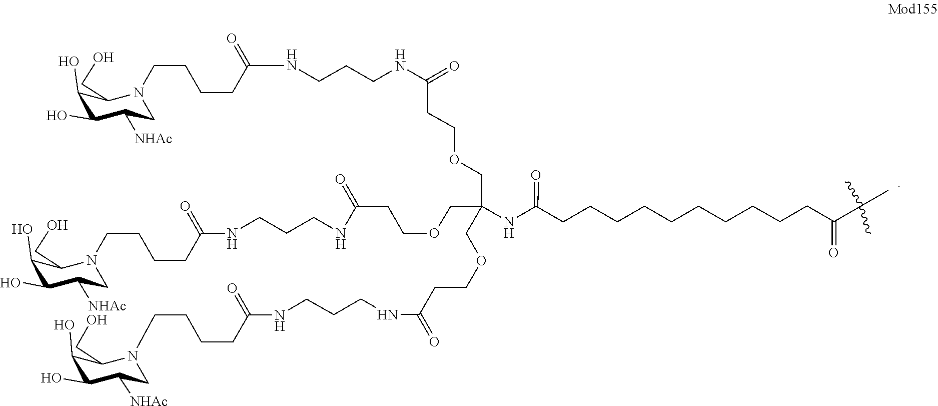

In some embodiments, an oligonucleotide comprises

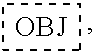

wherein each variable is independently as described herein. In some embodiments, each —OR′ is —OAc, and —N(R′)₂ is —NHAc. In some embodiments, an oligonucleotide comprises

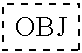

In some embodiments, each R′ is —H. In some embodiments, each —OR′ is —OH, and each —N(R′)₂ is —NHC(O)R. In some embodiments, each —OR′ is —OH, and each —N(R′)₂ is —NHAc. In some embodiments, an oligonucleotide comprises

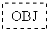

(L025). In some embodiments, the —CH₂— connection site is utilized as a C5 connection site in a sugar. In some embodiments, the connection site on the ring is utilized as a C3 connection site in a sugar. Such moieties may be introduced utilizing, e.g., phosphoramidites such as

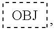

e.g.,

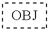

(those skilled in the art appreciate that one or more other groups, such as protection groups for —OH, —NH₂—, —N(i-Pr)₂, —OCH₂CH₂CN, etc., may be alternatively utilized, and protection groups can be removed under various suitable conditions, sometimes during oligonucleotide de-protection and/or cleavage steps). In some embodiments, an oligonucleotide comprises 2, 3 or more (e.g., 3 and no more than 3)

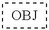

In some embodiments, an oligonucleotide comprises 2, 3 or more (e.g., 3 and no more than 3)

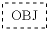

In some embodiments, copies of such moieties are linked by internucleotidic linkages, e.g., natural phosphate linkages, as described herein. In some embodiments, when at a 5′-end, a —CH₂— connection site is bonded to —OH. In some embodiments, an oligonucleotide comprises

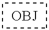

In some embodiments, an oligonucleotide comprises

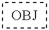

In some embodiments, each —OR′ is —OAc, and —N(R′)₂ is —NHAc. In some embodiments, an oligonucleotide comprises

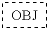

Among other things,

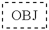

may be utilized to introduce

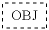

with comparable and/or better activities and/or properties. In some embodiments, it provides improved preparation efficiency and/or lower cost for the same number of

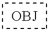

(e.g., when compared to Mod001).

In certain embodiments, an additional chemical moiety is a Mod group described herein, e.g., in Table 1.

In certain embodiments, an additional chemical moiety is Mod001. In certain embodiments, an additional chemical moiety is Mod083. In certain embodiments, an additional chemical moiety, e.g., a Mod group, is directly conjugated (e.g., without a linker) to the remainder of the ds oligonucleotide. In certain embodiments, an additional chemical moiety is conjugated via a linker to the remainder of the ds oligonucleotide. In certain embodiments, additional chemical moieties, e.g., Mod groups, may be directly connected, and/or via a linker, to nucleobases, sugars and/or internucleotidic linkages of ds oligonucleotides. In certain embodiments, Mod groups are connected, either directly or via a linker, to sugars. In certain embodiments, Mod groups are connected, either directly or via a linker, to 5′-end sugars. In certain embodiments, Mod groups are connected, either directly or via a linker, to 5′-end sugars via 5′ carbon. For examples, see various ds oligonucleotides in Table 1. In certain embodiments, Mod groups are connected, either directly or via a linker, to 3′-end sugars. In certain embodiments, Mod groups are connected, either directly or via a linker, to 3′-end sugars via 3′ carbon. In certain embodiments, Mod groups are connected, either directly or via a linker, to nucleobases. In certain embodiments, Mod groups are connected, either directly or via a linker, to internucleotidic linkages. In certain embodiments, provided oligonucleotides comprise Mod001 connected to 5′-end of oligonucleotide chains through L001.

As appreciated by those skilled in the art, an additional chemical moiety may be connected to a ds oligonucleotide chain at various locations, e.g., 5′-end, 3′-end, or a location in the middle (e.g., on a sugar, a base, an internucleotidic linkage, etc.). In certain embodiments, it is connected at a 5′-end. In certain embodiments, it is connected at a 3′-end. In certain embodiments, it is connected at a nucleotide in the middle.

Certain additional chemical moieties (e.g., lipid moieties, targeting moieties, carbohydrate moieties), including but not limited to Mod012, Mod039, Mod062, Mod085, Mod086, and Mod094, and various linkers for connecting additional chemical moieties to ds oligonucleotide chains, including but not limited to L001, L003, L004, L008, L009, and L010, are described in WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019032612, WO 2019/055951, WO 2019/075357, WO 2019/200185, WO 2019/217784, and/or WO 2019/032612, the additional chemical moieties and linkers of each of which are independently incorporated herein by reference, and can be utilized in accordance with the present disclosure. In certain embodiments, an additional chemical moiety is digoxigenin or biotin or a derivative thereof.

In certain embodiments, a ds oligonucleotide comprises a linker, e.g., L001 L004, L008, and/or an additional chemical moiety, e.g., Mod012, Mod039, Mod062, Mod085, Mod086, or Mod094. In certain embodiments, a linker, e.g., L001, L003, L004, L008, L009, L110, etc. is linked to a Mod, e.g., Mod012, Mod039, Mod062, Mod085, Mod086, Mod094, Mod152, Mod153, Mod154, Mod155 etc. L001: —NH—(CH₂)₆— linker (also known as a C₆ linker, C₆ amine linker or C₆ amino linker), connected to Mod, if any, through —NH—, and the 5′-end or 3′-end of the ds oligonucleotide chain through either a phosphate linkage (—O—P(O)(OH)—O—, which may exist as a salt form, and may be indicated as O or PO) or a phosphorothioate linkage (—O—P(O)(SH)—O—, which may exist as a salt form, and may be indicated as * if the phosphorothioate is not chirally controlled; or *S, S, or Sp, if the phosphorothioate is chirally controlled and has an Sp configuration, or *R, R, or Rp, if the phosphorothioate is chirally controlled and has an Rp configuration) as indicated at the —CH₂-connecting site. If no Mod is present, L001 is connected to —H through —NH—; L003:

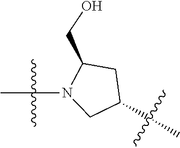 [SMILES: *[C@H]1C[C@H](CO)N(*)C1]

linker. In certain embodiments, it is connected to Mod, if any (if no Mod, —H), through its amino group, and the 5′-end or 3′-end of an oligonucleotide chain e.g., via a linkage (e.g., a phosphate linkage (O or PO) or a phosphorothioate linkage (can be either not chirally controlled or chirally controlled (Sp or Rp))); L004: linker having the structure of —NH(CH₂)₄CH(CH₂OH)CH₂—, wherein —NH— is connected to Mod (through —C(O)—) or —H, and the —CH₂— connecting site is connected to an oligonucleotide chain (e.g., at the 3′-end) through a linkage, e.g., phosphodiester (—O—P(O)(OH)—O—, which may exist as a salt form, and may be indicated as O or PO), phosphorothioate (—O—P(O)(SH)—O—, which may exist as a salt form, and may be indicated as * if the phosphorothioate is not chirally controlled; or *S, S, or Sp, if the phosphorothioate is chirally controlled and has an Sp configuration, or *R, R, or Rp, if the phosphorothioate is chirally controlled and has an Rp configuration), or phosphorodithioate (—O—P(S)(SH)—O—, which may exist as a salt form, and may be indicated as PS2 or: or D) linkage. For example, an asterisk immediately preceding a L004 (e.g., *L004) indicates that the linkage is a phosphorothioate linkage, and the absence of an asterisk immediately preceding L004 indicates that the linkage is a phosphodiester linkage. For example, in an oligonucleotide which terminates in . . . mAL004, the linker L004 is connected (via the —CH₂— site) through a phosphodiester linkage to the 3′ position of the 3′-terminal sugar (which is 2′-OMe modified and connected to the nucleobase A), and the L004 linker is connected via —NH— to —H. Similarly, in one or more oligonucleotides, the L004 linker is connected (via the —CH₂— site) through the phosphodiester linkage to the 3′ position of the 3′-terminal sugar, and the L004 is connected via —NH— to, e.g., Mod012, Mod085, Mod086, etc.; L008: linker having the structure of —C(O)—(CH₂)₉—, wherein —C(O)— is connected to Mod (through —NH—) or —OH (if no Mod indicated), and the —CH₂— connecting site is connected to an oligonucleotide chain (e.g., at the 5′-end) through a linkage, e.g., phosphodiester (—O—P(O)(OH)—O—, which may exist as a salt form, and may be indicated as O or PO), phosphorothioate (—O—P(O)(SH)—O—, which may exist as a salt form, and may be indicated as * if the phosphorothioate is not chirally controlled; or *S, S, or Sp, if the phosphorothioate is chirally controlled and has an Sp configuration, or *R, R, or Rp, if the phosphorothioate is chirally controlled and has an Rp configuration), or phosphorodithioate (—O—P(S)(SH)—O—, which may exist as a salt form, and may be indicated as PS2 or: or D) linkage. For example, in an example oligonucleotide which has the sequence of 5′-L008 mN * mN * mN * mN * N * N * N * N * N * N * N * N * N * N * mN * mN * mN * mN-3′, and which has a Stereochemistry/Linkage of OXXXXXXXXX XXXXXXXX, wherein N is a base, wherein O is a natural phosphate internucleotidic linkage, and wherein X is a stereorandom phosphorothioate, L008 is connected to —OH through —C(O)—, and the 5′-end of an oligonucleotide chain through a phosphate linkage (indicated as “O” in “Stereochemistry/Linkage”); in another example oligonucleotide, which has the sequence of 5′-Mod062L008 mN * mN * mN * mN * N * N * N * N * N * N * N * N * N * N * mN * mN * mN * mN-3′, and which has a Stereochemistry/Linkage of OXXXXXXXXX XXXXXXXX, wherein N is a base, L008 is connected to Mod062 through —C(O)—, and the 5′-end of an oligonucleotide chain through a phosphate linkage (indicated as “O” in “Stereochemistry/Linkage”);
L009: —CH₂CH₂CH₂—. In certain embodiments, when L009 is present at the 5′-end of an oligonucleotide without a Mod, one end of L009 is connected to —OH and the other end connected to a 5′-carbon of the oligonucleotide chain e.g., via a linkage (e.g., a phosphate linkage (O or PO) or a phosphorothioate linkage (can be either not chirally controlled or chirally controlled (Sp or Rp))); 75′

### L010:

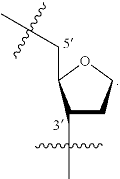

In certain embodiments, when L010 is present at the 5′-end of an oligonucleotide without a Mod, the 5′-carbon of L010 is connected to —OH and the 3′-carbon connected to a 5′-carbon of the oligonucleotide chain e.g., via a linkage (e.g., a phosphate linkage (O or PO) or a phosphorothioate linkage (can be either not chirally controlled or chirally controlled (Sp or Rp))); Mod012 (in certain embodiments, —C(O)— connects to —NH— of a linker such as L001, L004, L008, etc.):
L010 is utilized with n001R to form L010n001R, which has the structure of

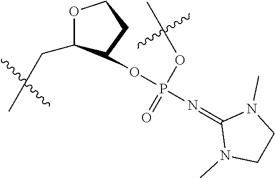

and wherein the configuration of linkage phosphorus is Rp. In some embodiments, multiple L010n001R may be utilized. For example, L023L010n001RL010n001RL010n001R, which has the following structure (which is bonded to the 5′-carbon at the 5′-end of the oligonucleotide chain, and each linkage phosphorus is independently Rp):

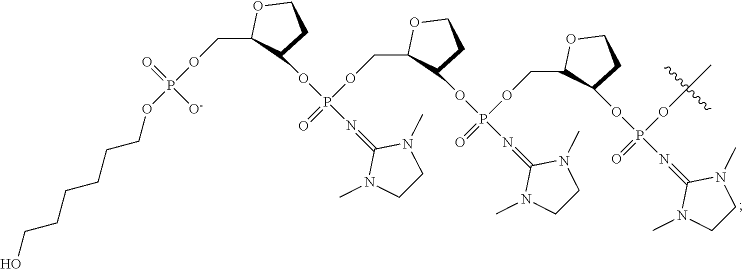

L023 is utilized with n001 to form L023n001, which has the structure of

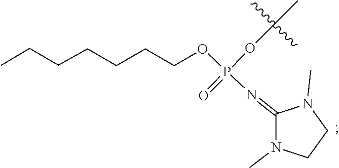 [SMILES: *OP(=O)(N=C1N(C)CCN1C)OCCCCCCC]

L023 is utilized with n009 to form L023n009, as in WV-42644 which has the structure of

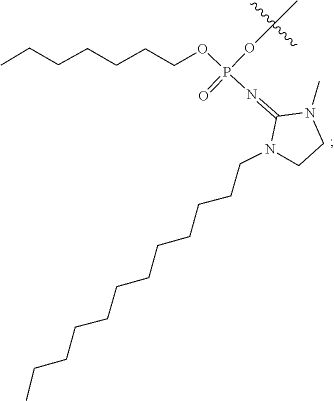 [SMILES: *OP(=O)(N=C1N(C)CCN1CCCCCCCCCCCC)OCCCCCCC]

In some embodiments, L023n001L009n001L009n001 may be utilized. For example, L023n001L009n001L009n001 as in WV-42643

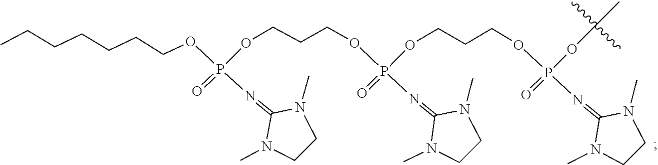 [SMILES: *OP(=O)(N=C1N(C)CCN1C)OCCCOP(=O)(N=C1N(C)CCN1C)OCCCOP(=O)(N=C1N(C)CCN1C)OCCCCCCC]

In some embodiments, L023n009L009n009 may be utilized. For example, as in WV-42646

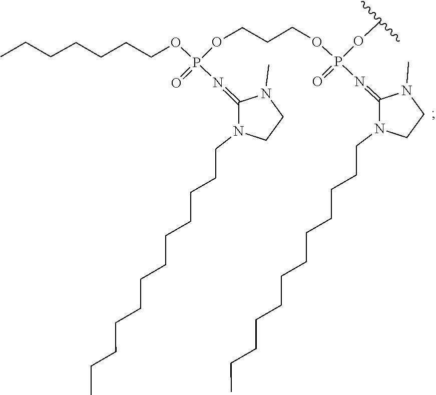 [SMILES: *OP(=O)(N=C1N(C)CCN1CCCCCCCCCCCC)OCCCOP(=O)(N=C1N(C)CCN1CCCCCCCCCCCC)OCCCCCCC]

In some embodiments, L023n009L009n009L009n009 may be utilized. For example, as in WV-42648

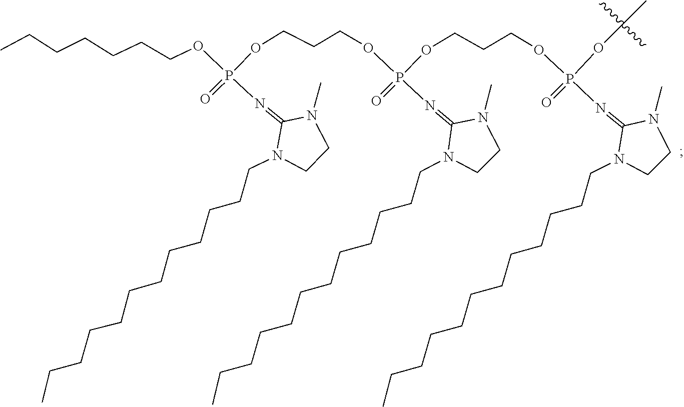 [SMILES: *OP(=O)(/N=C1\N(C)CCN1CCCCCCCCCCCC)OCCCOP(=O)(/N=C1\N(C)CCN1CCCCCCCCCCCC)OCCCOP(=O)(/N=C1\N(C)CCN1CCCCCCCCCCCC)OCCCCCCC]

In some embodiments L025 may be utilized; as in WV-41390,

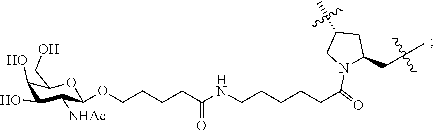

wherein the —CH₂— connection site is utilized as a C5 connection site of a sugar (e.g., a DNA sugar) and is connected to another unit (e.g., 3′ of a sugar), and the connection site on the ring is utilized as a C3 connection site and is connected to another unit (e.g., a 5′-carbon of a carbon), each of which is independently, e.g., via a linkage (e.g., a phosphate linkage (O or PO) or a phosphorothioate linkage (can be either not chirally controlled or chirally controlled (Sp or Rp))). When L025 is at a 5′-end without any modifications, its —CH₂— connection site is bonded to —OH. For example, L025L025L025—in various oligonucleotides has the structure of

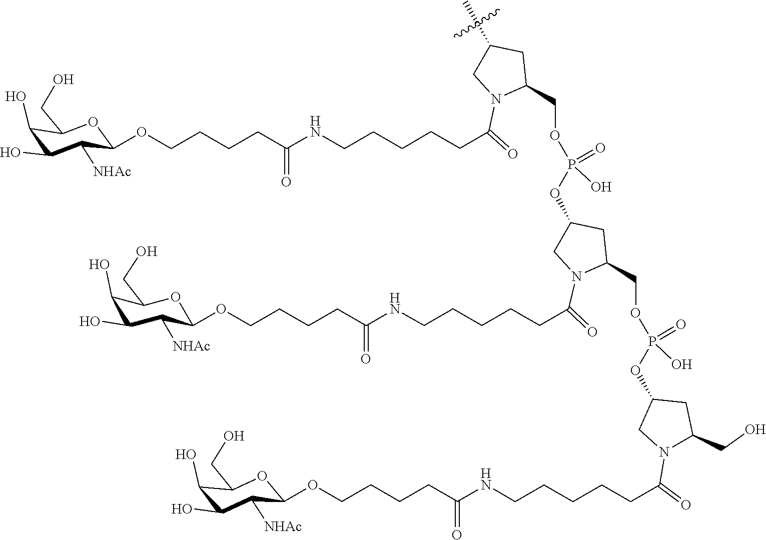

(may exist as various salt forms) and is connected to 5′-carbon of an oligonucleotide chain via a linkage as indicated (e.g., a phosphate linkage (O or PO) or a phosphorothioate linkage (can be either not chirally controlled or chirally controlled (Sp or Rp)));
In some embodiments L026 may be utilized; as in WV-44444,

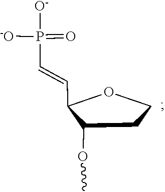

In some embodiments L027 may be utilized; as in WV-44445,

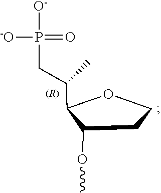

In some embodiments mU may be utilized; as in WV-42079,

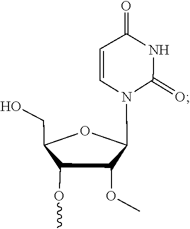

In some embodiments fU may be utilized; as in WV-44433,

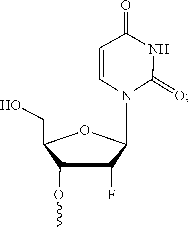

In some embodiments dT may be utilized; as in WV-44434,

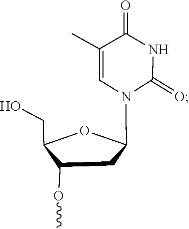

In some embodiments POdT or PO4-dT may be utilized; as in WV-44435,

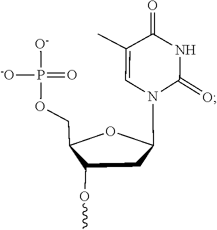

In some embodiments PO5MRdT may be utilized; as in WV-44436,

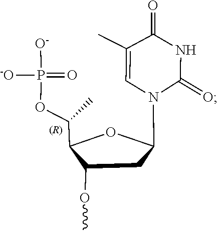

In some embodiments PO5MSdT may be utilized; as in WV-44437,

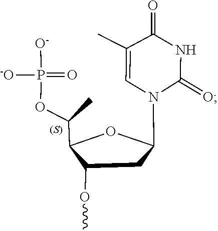

In some embodiments VPdT may be utilized; as in WV-44438,

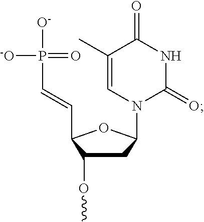

In some embodiments 5mvpdT may be utilized; as in WV-44439,

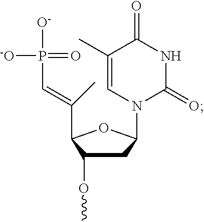

In some embodiments 5mrpdT may be utilized; as in WV-44440,

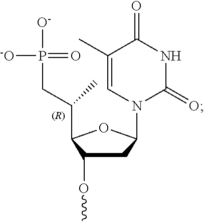

In some embodiments 5mspdT may be utilized; as in WV-44441,

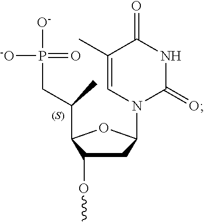

In some embodiments PNdT may be utilized; as in WV-44442,

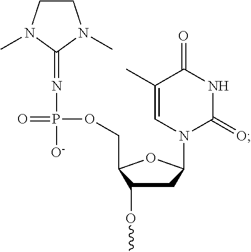

In some embodiments SPNdT may be utilized; as in WV-44443,

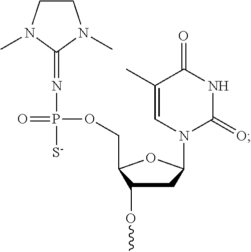

In some embodiments 5ptzdT may be utilized; as in WV-44446,

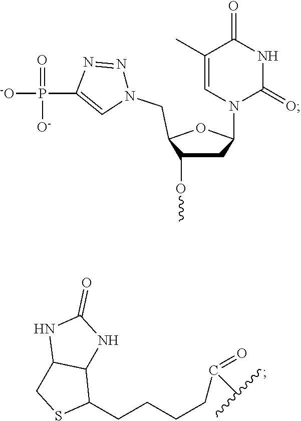

Mod039 (in certain embodiments, —C(O)— connects to —NH— of a linker such as L001, L003, L004, L008, L009, L110, etc.):

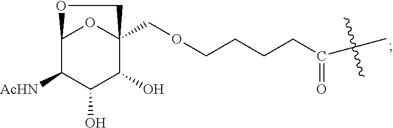 [SMILES: *C(=O)CCCCOC[C@@]12CO[C@@H](O1)[C@H](NC(C)=O)[C@@H](O)[C@H]2O]

Mod062 (in certain embodiments, —C(O)— connects to —NH— of a linker such as L001, L003, L004, L008, L009, L110, etc.):

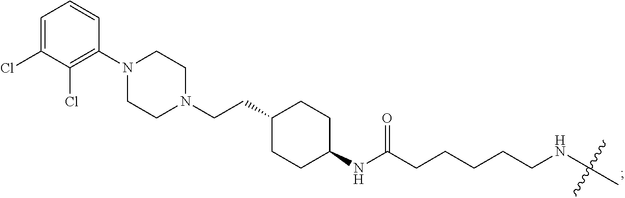 [SMILES: *NCCCCCC(=O)N[C@H]1CC[C@H](CCN2CCN(c3cccc(Cl)c3Cl)CC2)CC1]

Mod085 (in certain embodiments, —C(O)-connects to —NH— of a linker such as L001, L003, L004, L008, L009, L110, etc.):

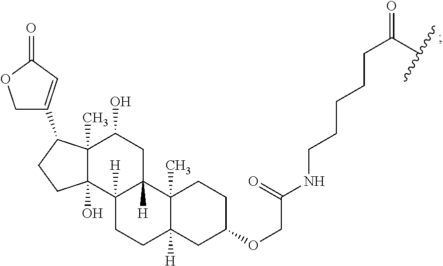

Mod086 (in certain embodiments, —C(O)— connects to —NH— of a linker such as L001, L003, L004, L008, L009, L110, etc.):

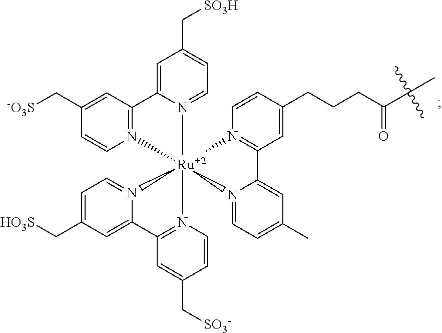

Mod094 (in certain embodiments, connects to an internucleotidic linkage, or to the 5′-end or 3′-end of an oligonucleotide via a linkage, e.g., a phosphate linkage, a phosphorothioate linkage (which is optionally chirally controlled), etc. For example, in an example oligonucleotide which has the sequence of 5′-mN * mN * mN * mN * N * N * N * N * N * N * N * N * N * N * mN * mN * mN * mNMod094-3′, and which has a Stereochemistry/Linkage of XXXXX XXXXX XXXXX XXO, wherein N is a base, Mod094 is connected to the 3′-end of the oligonucleotide chain (3′-carbon of the 3′-end sugar) through a phosphate group (which is not shown below and which may exist as a salt form; and which is indicated as “O” in “Stereochemistry/Linkage” ( . . . XXXXO))):

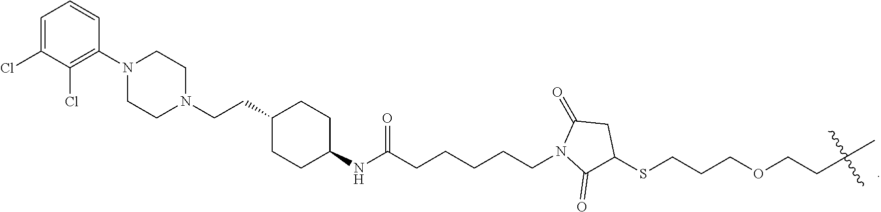 [SMILES: *CCOCCCSC1CC(=O)N(CCCCCC(=O)N[C@H]2CC[C@H](CCN3CCN(c4cccc(Cl)c4Cl)CC3)CC2)C1=O]

In certain embodiments, an additional chemical moiety is one described in WO 2012/030683. In certain embodiments, a provided ds oligonucleotide comprise a chemical structure (e.g., a linker, lipid, solubilizing group, and/or targeting ligand) described in WO 2012/030683.

In certain embodiments, a provided ds oligonucleotide comprises an additional chemical moiety and/or a modification (e.g., of nucleobase, sugar, internucleotidic linkage, etc.) described in: U.S. Pat. Nos. 5,688,941; 6,294,664; 6,320,017; 6,576,752; 5,258,506; 5,591,584; 4,958,013; 5,082,830; 5,118,802; 5,138,045; 6,783,931; 5,254,469; 5,414,077; 5,486,603; 5,112,963; 5,599,928; 6,900,297; 5,214,136; 5,109,124; 5,512,439; 4,667,025; 5,525,465; 5,514,785; 5,565,552; 5,541,313; 5,545,730; 4,835,263; 4,876,335; 5,578,717; 5,580,731; 5,451,463; 5,510,475; 4,904,582; 5,082,830; 4,762,779; 4,789,737; 4,824,941; 4,828,979; 5,595,726; 5,214,136; 5,245,022; 5,317,098; 5,371,241; 5,391,723; 4,948,882; 5,218,105; 5,112,963; 5,567,810; 5,574,142; 5,578,718; 5,608,046; 4,587,044; 4,605,735; 5,585,481; 5,292,873; 5,552,538; 5,512,667; 5,597,696; 5,599,923; 7,037,646; 5,587,371; 5,416,203; 5,262,536; 5,272,250; or 8,106,022.

In certain embodiments, an additional chemical moiety, e.g., a Mod, is connected via a linker. Various linkers are available in the art and may be utilized in accordance with the present disclosure, for example, those utilized for conjugation of various moieties with proteins (e.g., with antibodies to form antibody-drug conjugates), nucleic acids, etc. Certain useful linkers are described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, WO 2018/237194, WO 2019/032607, WO 2019/055951, WO 2019/075357, WO 2019/200185, WO 2019/217784, and/or WO 2019/032612, the linker moieties of each which are independently incorporated herein by reference. In certain embodiments, a linker is, as non-limiting examples, L001, L004, L009 or L010. In certain embodiments, an oligonucleotide comprises a linker, but not an additional chemical moiety other than the linker. In certain embodiments, a ds oligonucleotide comprises a linker, but not an additional chemical moiety other than the linker, wherein the linker is L001, L004, L009, or L010.

As demonstrated herein, provided technologies can provide high levels of activities and/or desired properties, in certain embodiments, without utilizing particular structural elements (e.g., modifications, linkage configurations and/or patterns, etc.) reported to be desired and/or necessary (e.g., those reported in WO 2019/219581), though certain such structural elements may be incorporated into ds oligonucleotides in combination with various other structural elements in accordance with the present disclosure. For example, in certain embodiments, ds oligonucleotides of the present disclosure have fewer nucleosides 3′ to a nucleoside opposite to a target nucleoside (e.g., a target adenosine), contain one or more phosphorothioate internucleotidic linkages at one or more positions where a phosphorothioate internucleotidic linkage was reportedly not favored or not allowed, contain one or more Sp phosphorothioate internucleotidic linkages at one or more positions where a Sp phosphorothioate internucleotidic linkage was reportedly not favored or not allowed, contain one or more Rp phosphorothioate internucleotidic linkages at one or more positions where a Rp phosphorothioate internucleotidic linkage was reportedly not favored or not allowed, and/or contain different modifications (e.g., internucleotidic linkage modifications, sugar modifications, etc.) and/or stereochemistry at one or more locations compared to those reportedly favorable or required for certain oligonucleotide properties and/or activities (e.g., presence of 2′-MOE, absence of phosphorothioate linkages at certain positions, absence of Sp phosphorothioate linkages at certain positions, and/or absence of Rp phosphorothioate linkages at certain positions were reportedly favorable or required for certain oligonucleotide properties and/or activities; as demonstrated herein, provided technologies can provide desired properties and/or high activities without utilizing 2′-MOE, without avoiding phosphorothioate linkages at one or more such certain positions, without avoiding Sp phosphorothioate linkages at one or more such certain positions, and/or without avoiding Rp phosphorothioate linkages at one or more such certain positions). Additionally or alternatively, provided ds oligonucleotides incorporates structural elements that were not previously recognized such as utilization of certain modifications (e.g., base modifications, sugar modifications (e.g., 2′-F), linkage modifications (e.g., non-negatively charged internucleotidic linkages), additional moieties, etc.) and levels, patterns, and combinations thereof.

For example, in certain embodiments, as described herein, provided d oligonucleotides contain no more than 5, 6, 7, 8, 9, 10, 11 or 12 nucleosides 3′ to a nucleoside opposite to a target nucleoside (e.g., a target adenosine).

Alternatively or additionally, as described herein (e.g., illustrated in certain Examples), for structural elements 3′ to a nucleoside opposite to a target nucleoside (e.g., a target adenosine), in certain embodiments, about 50%-100% (e.g., about or at least about 50%, 55%, 60%, 65%, 70%, 75%, 80%, 85%, 90%, 95%) of internucleotidic linkages 3′ to a nucleoside opposite to a target nucleoside (e.g., a target adenosine) are each independently a modified internucleotidic linkage, which is optionally chirally controlled. In certain embodiments, no more than 1, 2, or 3 internucleotidic linkages 3′ to a nucleoside opposite to a target nucleoside are natural phosphate linkages. In certain embodiments, no such internucleotidic linkage is natural phosphate linkages. In certain embodiments, no more than 1 such internucleotidic linkage is natural phosphate linkages. In certain embodiments, no more than 2 such internucleotidic linkages are natural phosphate linkages. In certain embodiments, no more than 3 such internucleotidic linkages are natural phosphate linkages. In certain embodiments, each modified internucleotidic linkage is independently a phosphorothioate or a non-negatively charged internucleotidic linkage (e.g., n001). In certain embodiments, each phosphorothioate internucleotidic linkage is chirally controlled. In certain embodiments, no more than 1, 2, or 3 internucleotidic linkages 3′ to a nucleoside opposite to a target nucleoside are Rp phosphorothioate internucleotidic linkage.

Alternatively or additionally, as described herein (e.g., illustrated in certain Examples), in certain embodiments, about 50%-100% (e.g., about or at least about 50%, 55%, 60%, 65%, 70%, 75%, 80%, 85%, 90%, or 95%) of internucleotidic linkages 5′ to a nucleoside opposite to a target nucleoside (e.g., a target adenosine) are each independently a modified internucleotidic linkage, which is optionally chirally controlled. In certain embodiments, no or no more than 1, 2, or 3 internucleotidic linkages 5′ to a nucleoside opposite to a target nucleoside (e.g., a target adenosine) are not modified internucleotidic linkages. In certain embodiments, no or no more than 1, 2, or 3 internucleotidic linkages 5′ to a nucleoside opposite to a target nucleoside (e.g., a target adenosine) are not phosphorothioate internucleotidic linkages. In certain embodiments, no or no more than 1, 2, or 3 internucleotidic linkages 5′ to a nucleoside opposite to a target nucleoside (e.g., a target adenosine) are not Sp phosphorothioate internucleotidic linkages. In certain embodiments, no more than 1, 2, or 3 internucleotidic linkages 5′ to a nucleoside opposite to a target nucleoside (e.g., a target adenosine) are natural phosphate linkages. In certain embodiments, no such internucleotidic linkage is natural phosphate linkages. In certain embodiments, no more than 1 such internucleotidic linkage is natural phosphate linkages. In certain embodiments, no more than 2 such internucleotidic linkages are natural phosphate linkages. In certain embodiments, no more than 3 such internucleotidic linkages are natural phosphate linkages. In certain embodiments, each modified internucleotidic linkage is independently a phosphorothioate or a non-negatively charged internucleotidic linkage (e.g., n001). In certain embodiments, there are no 2, 3, or 4 consecutive internucleotidic linkages 5′ to a nucleoside opposite to a target nucleoside, each of which is not a phosphorothioate internucleotidic linkage. In certain embodiments, there are no 2, 3, or 4 consecutive internucleotidic linkages 5′ to a nucleoside opposite to a target nucleoside, each of which is chirally controlled and is not a Sp phosphorothioate internucleotidic linkage. In certain embodiments, no or no more than 1, 2, 3, 4, or 5 internucleotidic linkages 5′ to a nucleoside opposite to a target nucleoside (e.g., a target adenosine) are Rp phosphorothioate internucleotidic linkage. In certain embodiments, at least about 1, 2, 3, 4, 5, 6, 7, 8, 9, or 10, or about 1, 2, 3, 4, 5, 6, 7, 8, 9, 10, 11, 12, 13, 14, 15, 16, 18, 19, 20, 21, 22, 23, 24, 25, 26, 27, 28, 29, 30, 31, or 32, or about 50%-100% (e.g., about or at least about 50%, 55%, 60%, 65%, 70%, 75%, 80%, 85%, 90%, or 95%) of internucleotidic linkages 5′ to a nucleoside opposite to a target nucleoside (e.g., a target adenosine) are each independently chirally controlled and a Sp internucleotidic linkage. In certain embodiments, at least about 1, 2, 3, 4, 5, 6, 7, 8, 9, or 10, or about 1, 2, 3, 4, 5, 6, 7, 8, 9, 10, 11, 12, 13, 14, 15, 16, 18, 19, 20, 21, 22, 23, 24, 25, 26, 27, 28, 29, 30, 31, or 32, or about 50%-100% (e.g., about or at least about 50%, 55%, 60%, 65%, 70%, 75%, 80%, 85%, 90%, or 95%) of phosphorothioate internucleotidic linkages 5′ to a nucleoside opposite to a target nucleoside (e.g., a target adenosine) are each independently chirally controlled and are Sp. In certain embodiments, each phosphorothioate internucleotidic linkages 5′ to a nucleoside opposite to a target nucleoside (e.g., a target adenosine) is chirally controlled. In certain embodiments, each phosphorothioate internucleotidic linkages 5′ to a nucleoside opposite to a target nucleoside (e.g., a target adenosine) is Sp.

### 6. Production of Oligonucleotides and Compositions

Various methods can be utilized for production of ds oligonucleotides and compositions and can be utilized in accordance with the present disclosure. For example, traditional phosphoramidite chemistry can be utilized to prepare stereorandom oligonucleotides and compositions, and certain reagents and chirally controlled technologies can be utilized to prepare chirally controlled oligonucleotide compositions, e.g., as described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, WO 2018/237194, WO 2019/032607, WO 2019/055951, WO 2019/075357, WO 2019/200185, WO 2019/217784, and/or WO 2019/032612, the reagents and methods of each of which is incorporated herein by reference.

In certain embodiments, chirally controlled/stereoselective preparation of ds oligonucleotides and compositions thereof comprise utilization of a chiral auxiliary, e.g., as part of monomeric phosphoramidites. Examples of such chiral auxiliary reagents and phosphoramidites are described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, WO 2018/237194, WO 2019/032607, WO 2019/055951, WO 2019/075357, WO 2019/200185, WO 2019/217784, and/or WO 2019/032612, the chiral auxiliary reagents and phosphoramidites of each of which are independently incorporated herein by reference. In certain embodiments, a chiral auxiliary is a chiral auxiliary described in any of: WO 2018/022473, WO 2018/098264, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612, the chiral auxiliaries of each of which are independently incorporated herein by reference.

In certain embodiments, chirally controlled preparation technologies, including oligonucleotide synthesis cycles, reagents and conditions are described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, and/WO 2018/098264, WO 2018/022473, WO 2018/223056, WO 2018/223073, WO 2018/223081, WO 2018/237194, WO 2019/032607, WO2019032612, WO 2019/055951, WO 2019/075357, WO 2019/200185, WO 2019/217784, and/or WO 2019/032612, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612, the oligonucleotide synthesis methods, cycles, reagents and conditions of each of which are independently incorporated herein by reference.

Once synthesized, provided ds oligonucleotides and compositions are typically further purified. Suitable purification technologies are widely known and practiced by those skilled in the art, including but not limited to those described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, WO 2018/237194, WO 2019/032607, WO 2019/055951, WO 2019/075357, WO 2019/200185, WO 2019/217784, and/or WO 2019/032612, the purification technologies of each of which are independently incorporated herein by reference.

In certain embodiments, a cycle comprises or consists of coupling, capping, modification and deblocking. In certain embodiments, a cycle comprises or consists of coupling, capping, modification, capping and deblocking. These steps are typically performed in the order they are listed, but in certain embodiments, as appreciated by those skilled in the art, the order of certain steps, e.g., capping and modification, may be altered. If desired, one or more steps may be repeated to improve conversion, yield and/or purity as those skilled in the art often perform in syntheses. For example, in certain embodiments, coupling may be repeated; in certain embodiments, modification (e.g., oxidation to install ═O, sulfurization to install ═S, etc.) may be repeated; in certain embodiments, coupling is repeated after modification which can convert a P(III) linkage to a P(V) linkage which can be more stable under certain circumstances, and coupling is routinely followed by modification to convert newly formed P(III) linkages to P(V) linkages. In certain embodiments, when steps are repeated, different conditions may be employed (e.g., concentration, temperature, reagent, time, etc.).

Technologies for formulating provided ds oligonucleotides and/or preparing pharmaceutical compositions, e.g., for administration to subjects via various routes, are readily available in the art and can be utilized in accordance with the present disclosure, e.g., those described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, or WO 2018/237194 and references cited therein.

Technologies for formulating provided ds oligonucleotides and/or preparing pharmaceutical compositions, e.g., for administration to subjects via various routes, are readily available in the art and can be utilized in accordance with the present disclosure, e.g., those described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, or WO 2018/237194 and references cited therein.

In certain embodiments, a useful chiral auxiliary has the structure of

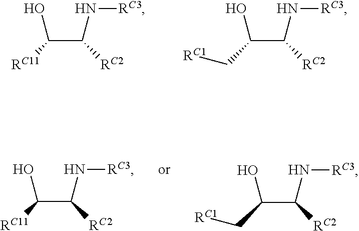

or a salt thereof, wherein R^{C11} is -L^{C1}-R^{C1}, L^{C1} is optionally substituted —CH₂—. R^{C1} is R, —Si(R)₃, —SO₂R or an electron-withdrawing group, and R^{C2} and R^{C3} are taken together with their intervening atoms to form an optionally substituted 3-10 membered saturated ring having, in addition to the nitrogen atom, 0-2 heteroatoms. In certain embodiments, a useful chiral auxiliary has the structure of

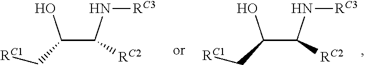

wherein R^{C1} is R, —Si(R)₃ or —SO₂R, and R^{C2} and R^{C3} are taken together with their intervening atoms to form an optionally substituted 3-7 membered saturated ring having, in addition to the nitrogen atom, 0-2 heteroatoms. is a formed ring is an optionally substituted 5-membered ring. In certain embodiments, a useful chiral auxiliary has the structure of

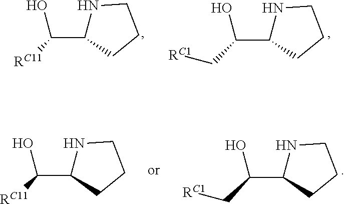

or a salt thereof. In certain embodiments, a useful chiral auxiliary has the structure of

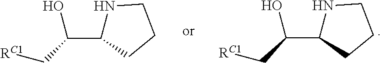

In certain embodiments, a useful chiral auxiliary is a DPSE chiral auxiliary. In certain embodiments, purity or stereochemical purity of a chiral auxiliary is at least 85%, 90%, 95%, 96%, 97%, 98%, or 99%. In certain embodiments, it is at least 85%. In certain embodiments, it is at least 90%. In certain embodiments, it is at least 95%. In certain embodiments, it is at least 96%. In certain embodiments, it is at least 97%. In certain embodiments, it is at least 98%. In certain embodiments, it is at least 99%.

In certain embodiments, L^{C1} is —CH₂—. In certain embodiments, L^{cl} is substituted —CH₂—. In certain embodiments, L^{C1} is monosubstituted —CH₂—.

In certain embodiments, R^{C1} is R. In certain embodiments, R^{C1} is optionally substituted phenyl. In certain embodiments, R^{C1} is —SiR₃. In certain embodiments, R^{C1} is —SiPh₂Me. In certain embodiments, R^{C1} is —SO₂R. In certain embodiments, R is not hydrogen. In certain embodiments, R is optionally substituted phenyl. In certain embodiments, R is phenyl. In certain embodiments, R is optionally substituted C₁₋₆ aliphatic. In certain embodiments, R is C₁₋₆ alkyl. In certain embodiments, R is methyl. In certain embodiments, R is t-butyl.

In certain embodiments, R^{C1} is an electron-withdrawing group, such as —C(O)R, —OP(O)(OR)₂, —OP(O)(R)₂, —P(O)(R)₂, —S(O)R, —S(O)₂R, etc. In certain embodiments, chiral auxiliaries comprising electron-withdrawing group R^{C1} groups are particularly useful for preparing chirally controlled non-negatively charged internucleotidic linkages and/or chirally controlled internucleotidic linkages bonded to natural RNA sugar.

In certain embodiments, R^{C2} and R^{C3} are taken together with their intervening atoms to form an optionally substituted 3-10 (e.g., 3, 4, 5, 6, 7, 8, 9, or 10) membered saturated ring having no heteroatoms in addition to the nitrogen atom. In certain embodiments, R^{C2} and R^{C3} are taken together with their intervening atoms to form an optionally substituted 5-membered saturated ring having no heteroatoms in addition to the nitrogen atom.

In certain embodiments, the present disclosure provides useful reagents for preparation of ds oligonucleotides and compositions thereof. In certain embodiments, phosphoramidites comprise nucleosides, nucleobases and sugars as described herein. In certain embodiments, nucleobases and sugars are properly protected for oligonucleotide synthesis as those skilled in the art will appreciate. In certain embodiments, a phosphoramidite has the structure of R^{NS}—P(OR)N(R)₂, wherein R^{N}S is a optionally protected nucleoside moiety. In certain embodiments, a phosphoramidite has the structure of R^{NS}—P(OCH₂CH₂CN)N(i-Pr)₂. In certain embodiments, a phosphoramidite comprises a nucleobase which is or comprises Ring BA, wherein Ring BA has the structure of BA-I, BA-I-a, BA-I-b, BA-II, BA-II-a, BA-II-b, BA-III, BA-III-a, BA-III-b, BA-IV, BA-IV-a, BA-IV-b, BA-V, BA-V-a, BA-V-b, or BA-VI, or a tautomer of Ring BA, wherein the nucleobase is optionally substituted or protected. In certain embodiments, a phosphoramidite comprises a chiral auxiliary moiety, wherein the phosphorus is bonded to an oxygen and a nitrogen atom of the chiral auxiliary moiety. In certain embodiments, a phosphoramidite has the structure of

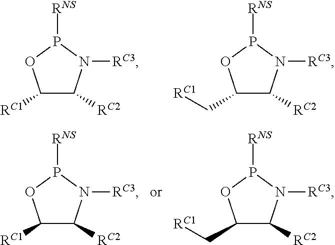

or a salt thereof, wherein RNS is a protected nucleoside moiety (e.g., 5′-OH and/or nucleobases suitably protected for oligonucleotide synthesis), and each other variable is independently as described herein. In certain embodiments, a phosphoramidite has the structure of

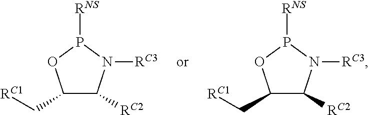

wherein RNS is a protected nucleoside moiety (e.g., 5′-OH and/or nucleobases suitably protected for oligonucleotide synthesis), R^{C1} is R, —Si(R)₃ or —SO₂R, and R^{C2} and R^{C3} are taken together with their intervening atoms to form an optionally substituted 3-7 membered saturated ring having, in addition to the nitrogen atom, 0-2 heteroatoms, wherein the coupling forms an internucleotidic linkage. In certain embodiments, 5′-OH of R^{N}S is protected. In certain embodiments, 5′-OH of R^{N}S is protected as —ODMTr. In certain embodiments, R^{N}S is bonded to phosphorus through its 3′-O—. In certain embodiments, a formed ring by R^{C2} and R^{C3} is an optionally substituted 5-membered ring. In certain embodiments, a phosphoramidite has the structure of

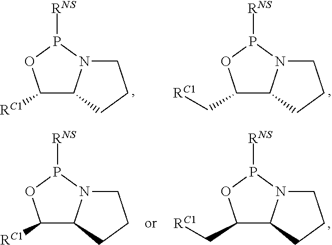

or a salt thereof. In certain embodiments, a phosphoramidite has the structure of

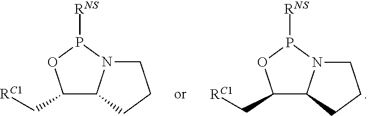

In certain embodiments, purity or stereochemical purity of a phosphoramidite is at least 85%, 90%, 95%, 96%, 97%, 98%, or 99%. In certain embodiments, it is at least 85%. In certain embodiments, it is at least 90%. In certain embodiments, it is at least 95%.

In certain embodiments, the present disclosure provides a method for preparing an oligonucleotide or composition, comprising coupling a free —OH, e.g., a free 5′-OH, of an oligonucleotide or a nucleoside with a phosphoramidite as described herein.

In certain embodiments, the present disclosure provides an oligonucleotide, wherein the oligonucleotide comprises one or more modified internucleotidic linkages each independently having the structure of —O⁵—P^{L}(W)(R^{CA})—O³—, wherein:
P^{L} is P, or P(═W);
W is O, S, or W^{N};
W^{N} is ═N—C(—N(R¹)₂═N⁺(R¹)₂Q-;
Q⁻ is an anion;
R^{CA} is or comprises an optionally capped chiral auxiliary moiety,
O is an oxygen bonded to a 5′-carbon of a sugar, and
O³ is an oxygen bonded to a 3′-carbon of a sugar.

In certain embodiments, a modified internucleotidic linkage is optionally chirally controlled. In certain embodiments, a modified internucleotidic linkage is optionally chirally controlled.

In certain embodiments, a provided methods comprising removing R^{CA} from such a modified internucleotidic linkages. In certain embodiments, after removal, bonding to R^{CA} is replaced with —OH. In certain embodiments, after removal, bonding to RCA is replaced with ═O, and bonding to W^{N} is replaced with —N═C(N(R¹)₂)₂.

In certain embodiments, P^{L} is P═S, and when R^{CA} is removed, such an internucleotidic linkage is converted into a phosphorothioate internucleotidic linkage.

In certain embodiments, P^{L} is P═W^{N}, and when R^{CA} is removed, such an internucleotidic linkage is converted into an internucleotidic linkage having the structure of

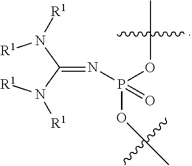

In certain embodiments, an internucleotidic linkage having the structure of

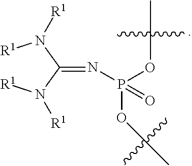

has the structure of

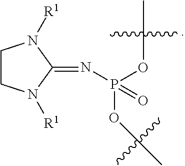

In certain embodiments, an internucleotidic linkage having the structure of

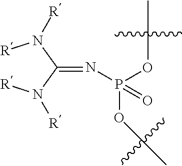

has the structure of

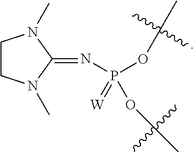

In certain embodiments, P^{L} is P (e.g., in newly formed internucleotidic linkage from coupling of a phosphoramidite with a 5′-OH). In certain embodiments, W is O or S. In certain embodiments, W is S (e.g., after sulfurization). In certain embodiments, W is O (e.g., after oxidation). In certain embodiments, certain non-negatively charged internucleotidic linkages or neutral internucleotidic linkages may be prepared by reacting a P(III) phosphite triester internucleotidic linkage with azido imidazolinium salts (e.g., compounds comprising

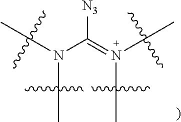

under suitable conditions. In certain embodiments, an azido imidazolinium salt is a salt of PF₆⁻. In certain embodiments, an azido imidazolinium salt is a slat of

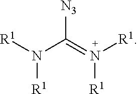

In certain embodiments, an azido imidazolinium salt is 2-azido-1,3-dimethylimidazolinium hexafluorophosphate.

As appreciated by those skilled in the art, Q⁻ can be various suitable anion present in a system (e.g., in oligonucleotide synthesis), and may vary during oligonucleotide preparation processes depending on cycles, process stages, reagents, solvents, etc. In certain embodiments, Q⁻ is PF₆⁻.

In certain embodiments, R^{CA} is

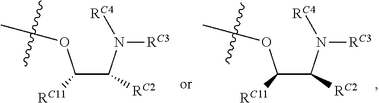

wherein R^{C4} is —H or —C(O)R′, and each other variable is independently as described herein. In certain embodiments, R^{CA} is

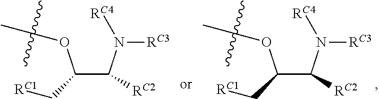

wherein R^{C1} is R, —Si(R)₃ or —SO₂R, R^{C2} and R^{C3} are taken together with their intervening atoms to form an optionally substituted 3-7 membered saturated ring having, in addition to the nitrogen atom, 0-2 heteroatoms, R^{C4} is —H or —C(O)R′. In certain embodiments, R^{C4} is —H. In certain embodiments, R^{C4} is —C(O)CH₃. In certain embodiments, R^{C2} and R^{C3} are taken together to form an optionally substituted 5-membered ring.

In certain embodiments, R^{C4} is —H (e.g., in n newly formed internucleotidic linkage from coupling of a phosphoramidite with a 5′-OH). In certain embodiments, R^{C4} is —C(O)R (e.g., after capping of the amine). In certain embodiments, R is methyl.

In certain embodiments, each chirally controlled phosphorothioate internucleotidic linkage is independently converted from —O⁵—P^{L}(W)(R^{CA})—O³—.

### 8. Characterization and Assessment

In certain embodiments, properties and/or activities of dsRNAi oligonucleotides and compositions thereof can be characterized and/or assessed using various technologies available to those skilled in the art, e.g., biochemical assays, cell based assays, animal models, clinical trials, etc.

In certain embodiments, a method of identifying and/or characterizing an oligonucleotide composition, e.g., a dsRNAi oligonucleotide composition, comprises steps of: providing at least one composition comprising a plurality of oligonucleotides; and assessing delivery relative to a reference composition.

In certain embodiments, the present disclosure provides a method of identifying and/or characterizing a ds oligonucleotide composition, e.g., a dsRNAi oligonucleotide composition, comprises steps of: providing at least one composition comprising a plurality of ds oligonucleotides; and assessing cellular uptake relative to a reference composition.

In certain embodiments, the present disclosure provides a method of identifying and/or characterizing a ds oligonucleotide composition, e.g., a dsRNAi oligonucleotide composition, comprises steps of: providing at least one composition comprising a plurality of ds oligonucleotides; and assessing reduction of transcripts of a target gene and/or a product encoded thereby relative to a reference composition.

In certain embodiments, the present disclosure provides a method of identifying and/or characterizing a ds oligonucleotide composition, e.g., a dsRNAi oligonucleotide composition, comprises steps of: providing at least one composition comprising a plurality of ds oligonucleotides; and assessing reduction of tau levels, its aggregation and/or spreading relative to a reference composition.

In certain embodiments, properties and/or activities of ds oligonucleotides, e.g., dsRNAi oligonucleotides, and compositions thereof are compared to reference ds oligonucleotides and compositions thereof, respectively.

In certain embodiments, a reference ds oligonucleotide composition is a stereorandom ds oligonucleotide composition. In certain embodiments, a reference ds oligonucleotide composition is a stereorandom composition of ds oligonucleotides of which all internucleotidic linkages are phosphorothioate. In certain embodiments, a reference ds oligonucleotide composition is a ds DNA oligonucleotide composition with all phosphate linkages. In certain embodiments, a reference ds oligonucleotide composition is otherwise identical to a provided chirally controlled ds oligonucleotide composition except that it is not chirally controlled. In certain embodiments, a reference ds oligonucleotide composition is otherwise identical to a provided chirally controlled oligonucleotide composition except that it has a different pattern of stereochemistry. In certain embodiments, a reference ds oligonucleotide composition is similar to a provided ds oligonucleotide composition except that it has a different modification of one or more sugar, base, and/or internucleotidic linkage, or pattern of modifications. In certain embodiments, a ds oligonucleotide composition is stereorandom and a reference ds oligonucleotide composition is also stereorandom, but they differ in regard to sugar and/or base modification(s) or patterns thereof.

In certain embodiments, a reference composition is a composition of ds oligonucleotides having the same base sequence and the same chemical modifications. In certain embodiments, a reference composition is a composition of ds oligonucleotides having the same base sequence and the same pattern of chemical modifications. In certain embodiments, a reference composition is a non-chirally controlled (or stereorandom) composition of ds oligonucleotides having the same base sequence and chemical modifications. In certain embodiments, a reference composition is a non-chirally controlled (or stereorandom) composition of ds oligonucleotides of the same constitution but is otherwise identical to a provided chirally controlled ds oligonucleotide composition.

In certain embodiments, a reference ds oligonucleotide composition is of ds oligonucleotides having a different base sequence. In certain embodiments, a reference ds oligonucleotide composition is of ds oligonucleotides that do not target RNAi (e.g., as negative control for certain assays).

In certain embodiments, a reference composition is a composition of ds oligonucleotides having the same base sequence but different chemical modifications, including but not limited to chemical modifications described herein. In certain embodiments, a reference composition is a composition of ds oligonucleotides having the same base sequence but different patterns of internucleotidic linkages and/or stereochemistry of internucleotidic linkages and/or chemical modifications.

Various methods are known in the art for detection of gene products, the expression, level and/or activity of which may be altered after introduction or administration of a provided ds oligonucleotide. For example, transcripts and their knockdown can be detected and quantified with qPCR, and protein levels can be determined via Western blot.

In certain embodiments, assessment of efficacy of ds oligonucleotides can be performed in biochemical assays or in vitro in cells. In certain embodiments, dsRNAi oligonucleotides can be introduced to cells via various methods available to those skilled in the art, e.g., gymnotic delivery, transfection, lipofection, etc.

In certain embodiments, the efficacy of a putative dsRNAi oligonucleotide can be tested in vitro.

In certain embodiments, the efficacy of a putative dsRNAi oligonucleotide can be tested in vitro using any known method of testing the expression, level and/or activity of a gene or gene product thereof.

In certain embodiments, dsRNAi soluble aggregates can be observed by immunoblotting.

In certain embodiments, a dsRNAi oligonucleotide is tested in a cell or animal model of a disease.

In certain embodiments, an animal model administered a dsRNAi oligonucleotide can be evaluated for safety and/or efficacy.

In certain embodiments, the effect(s) of administration of a ds oligonucleotide to an animal can be evaluated, including any effects on behavior, inflammation, and toxicity. In certain embodiments, following dosing, animals can be observed for signs of toxicity including trouble grooming, lack of food consumption, and any other signs of lethargy. In certain embodiments, in a mouse model, following administration of a dsRNAi oligonucleotide, the animals can be monitored for timing of onset of a rear paw clasping phenotype.

In certain embodiments, following administration of a dsRNAi oligonucleotide to an animal, the animal can be sacrificed and analysis of tissues or cells can be performed to determine changes in RNAi activity, or other biochemical or other changes. In certain embodiments, following necropsy, liver, heart, lung, kidney, and spleen can be collected, fixed, and processed for histopathological evaluation (standard light microscopic examination of hematoxylin and eosin-stained tissue slides).

In certain embodiments, following administration of a dsRNAi oligonucleotide to an animal, behavioral changes can be monitored or assessed. In certain embodiments, such an assessment can be performed using a technique described in the scientific literature.

Various effects of testing in animals described herein can also be monitored in human subjects or patients following administration of a dsRNAi oligonucleotide.

In addition, the efficacy of a dsRNAi oligonucleotide in a human subject can be measured by evaluating, after administration of the oligonucleotide, any of various parameters known in the art, including but not limited to a reduction in a symptom, or a decrease in the rate of worsening or onset of a symptom of a disease.

In certain embodiments, following human treatment with a ds oligonucleotide, or contacting a cell or tissue in vitro with an oligonucleotide, cells and/or tissues are collected for analysis.

In certain embodiments, in various cells and/or tissues, target nucleic acid levels can be quantitated by methods available in the art, many of which can be accomplished with commercially available kits and materials. Such methods include, e.g., Northern blot analysis, competitive polymerase chain reaction (PCR), quantitative real-time PCR, etc. RNA analysis can be performed on total cellular RNA or poly(A)+mRNA. Probes and primers are designed to hybridize to a nucleic acid to be detected. Methods for designing real-time PCR probes and primers are well known and widely practiced in the art. For example, to detect and quantify RNAi RNA, an example method comprises isolation of total RNA (e.g., including mRNA) from a cell or animal treated with an oligonucleotide or a composition and subjecting the RNA to reverse transcription and/or quantitative real-time PCR, for example, as described herein, or in: Moon et al. 2012 Cell Metab. 15: 240-246.

In certain embodiments, protein levels can be evaluated or quantitated in various methods known in the art, e.g., enzyme-linked immunosorbent assay (ELISA), Western blot analysis (immunoblotting), immunocytochemistry, fluorescence-activated cell sorting (FACS), immuno-histochemistry, immunoprecipitation, protein activity assays (for example, caspase activity assays), and quantitative protein assays. Antibodies useful for the detection of mouse, rat, monkey, and human proteins are commercially available or can be generated if needed. For example, various RNAi antibodies have been reported.

Various technologies are available and/or known in the art for detecting levels of ds oligonucleotides or other nucleic acids. Such technologies are useful for detecting dsRNAi oligonucleotides when administered to assess, e.g., delivery, cell uptake, stability, distribution, etc.

In certain embodiments, selection criteria are used to evaluate the data resulting from various assays and to select particularly desirable ds oligonucleotides, e.g., desirable dsRNAi oligonucleotides, with certain properties and activities. In certain embodiments, selection criteria include an IC₅₀ of less than about 10 nM, less than about 5 nM or less than about 1 nM. In certain embodiments, selection criteria for a stability assay include at least 50% stability [at least 50% of an oligonucleotide is still remaining and/or detectable] at Day 1. In certain embodiments, selection criteria for a stability assay include at least 50% stability at Day 2. In certain embodiments, selection criteria for a stability assay include at least 50% stability at Day 3. In certain embodiments, selection criteria for a stability assay include at least 50% stability at Day 4. In certain embodiments, selection criteria for a stability assay include at least 50% stability at Day 5. In certain embodiments, selection criteria for a stability assay include at least 80% [at least 80% of the oligonucleotide remains] at Day 5.

In certain embodiments, efficacy of a dsRNAi oligonucleotide is assessed directly or indirectly by monitoring, measuring or detecting a change in a condition, disorder or disease or a biological pathway.

In certain embodiments, efficacy of a dsRNAi oligonucleotide is assessed directly or indirectly by monitoring, measuring or detecting a change in a response to be affected by knockdown.

In certain embodiments, a provided ds oligonucleotide (e.g., a dsRNAi oligonucleotide) can by analyzed by a sequence analysis to determine what other genes (e.g., genes which are not a target gene) have a sequence which is complementary to the base sequence of the provided ds oligonucleotide (e.g., the dsRNAi oligonucleotide) or which have 0, 1, 2 or more mismatches from the base sequence of the provided ds oligonucleotide (e.g., the dsRNAi oligonucleotide). Knockdown, if any, by the ds oligonucleotide of these potential off-targets can be determined to evaluate potential off-target effects of a ds oligonucleotide (e.g., a dsRNAi oligonucleotide). In certain embodiments, an off-target effect is also termed an unintended effect and/or related to hybridization to a bystander (non-target) sequence or gene.

In certain embodiments, a dsRNAi oligonucleotide which has been evaluated and tested for its ability to provide a particular biological effect (e.g., reduction of level, expression and/or activity of a target gene or a gene product thereof) can be used to treat, ameliorate and/or prevent a condition, disorder or disease.

### 9. Biologically Active Oligonucleotides

In certain embodiments, the present disclosure encompasses ds oligonucleotides which capable of acting as dsRNAi agents.

In certain embodiments, provided compositions include one or more oligonucleotides fully or partially complementary to a strand of: structural genes, genes control and/or termination regions, and/or self-replicating systems such as viral or plasmid DNA. In certain embodiments, provided compositions include one or more oligonucleotides that are or act as RNAi agents or other RNA interference reagents (RNAi agents or iRNA agents), shRNA, antisense oligonucleotides, self-cleaving RNAs, ribozymes, fragment thereof and/or variants thereof (such as Peptidyl transferase 23S rRNA, RNase P, Group I and Group II introns, GIR1 branching ribozymes, Leadzyme, Hairpin ribozymes, Hammerhead ribozymes, HDV ribozymes, Mammalian CPEB3 ribozyme, VS ribozymes, glmS ribozymes, CoTC ribozyme, etc.), microRNAs, microRNA mimics, supermirs, aptamers, antimirs, antagomirs, Ul adaptors, triplex-forming oligonucleotides, RNA activators, long non-coding RNAs, short non-coding RNAs (e.g., piRNAs), immunomodulatory oligonucleotides (such as immunostimulatory oligonucleotides, immunoinhibitory oligonucleotides), GNA, LNA, ENA, PNA, TNA, morpholinos, G-quadruplex (RNA and DNA), antiviral oligonucleotides, and decoy oligonucleotides.

In certain embodiments, provided compositions include one or more hybrid (e.g., chimeric) oligonucleotides. In the context of the present disclosure, the term “hybrid” broadly refers to mixed structural elements of oligonucleotides. Hybrid oligonucleotides may refer to, for example, (1) an oligonucleotide molecule having mixed classes of nucleotides, e.g., part DNA and part RNA within the single molecule (e.g., DNA-RNA); (2) complementary pairs of nucleic acids of different classes, such that DNA:RNA base pairing occurs either intramolecularly or intermolecularly; or both; (3) an oligonucleotide with two or more kinds of the backbone or internucleotide linkages.

In certain embodiments, provided compositions include one or more oligonucleotide that comprises more than one classes of nucleic acid residues within a single molecule. For example, in any of the embodiments described herein, an oligonucleotide may comprise a DNA portion and an RNA portion. In certain embodiments, an oligonucleotide may comprise a unmodified portion and modified portion.

Provided ds oligonucleotide compositions can include oligonucleotides containing any of a variety of modifications, for example as described herein. In certain embodiments, particular modifications are selected, for example, in light of intended use. In certain embodiments, it is desirable to modify one or both strands of a double-stranded oligonucleotide (or a double-stranded portion of a single-stranded oligonucleotide). In certain embodiments, the two strands (or portions) include different modifications. In certain embodiments, the two strands include the same modifications. One of skill in the art will appreciate that the degree and type of modifications enabled by methods of the present disclosure allow for numerous permutations of modifications to be made. Examples of such modifications are described herein and are not meant to be limiting.

The phrase “antisense strand” or “guide strand” as used herein, refers to an oligonucleotide that is substantially or 100% complementary to a target sequence of interest. The phrase “antisense strand” or “guide strand” includes the antisense region of both oligonucleotides that are formed from two separate strands, as well as unimolecular oligonucleotides that are capable of forming hairpin or dumbbell type structures. In reference to a double-stranded RNAi agent such as a siRNA, the antisense strand is the strand preferentially incorporated into RISC, and which targets RISC-mediated knockdown of a RNA target. In reference to a double-stranded RNAi agent, the terms “antisense strand” and “guide strand” are used interchangeably herein; and the terms “sense strand” or “passenger strand” are used interchangeably herein in reference to the strand which is not the antisense strand.

The phrase “sense strand” refers to an oligonucleotide that has the same nucleoside sequence, in whole or in part, as a target sequence such as a messenger RNA or a sequence of DNA.

By “target sequence” is meant any nucleic acid sequence whose expression or activity is to be modulated. The target nucleic acid can be DNA or RNA, such as endogenous DNA or RNA, viral DNA or viral RNA, or other RNA encoded by a gene, virus, bacteria, fungus, mammal, or plant. In certain embodiments, a target sequence is associated with a disease or disorder. In reference to RNA interference and RNase H-mediated knockdown, a target sequence is generally a RNA target sequence.

By “specifically hybridizable” and “complementary” is meant that a nucleic acid can form hydrogen bond(s) with another nucleic acid sequence by either traditional Watson-Crick or other non-traditional types. In reference to the nucleic molecules of the present disclosure, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al, 1987, CSH Symp. Quant. Biol. LIT pp. 123-133; Frier et al., 1986, Proc. Nat. Acad. Sci. USA83:9373-9377; Turner et al., 1987, J. Am. Chem. Soc. 109:3783-3785)

A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” or 100% complementarity means that all the contiguous residues of a nucleic acid sequence will hydrogen bond with the same number of contiguous residues in a second nucleic acid sequence. Less than perfect complementarity refers to the situation in which some, but not all, nucleoside units of two strands can hydrogen bond with each other. “Substantial complementarity” refers to polynucleotide strands exhibiting 90% or greater complementarity, excluding regions of the polynucleotide strands, such as overhangs, that are selected so as to be noncomplementary. Specific binding requires a sufficient degree of complementarity to avoid non-specific binding of the oligomeric compound to non-target sequences under conditions in which specific binding is desired, e.g., under physiological conditions in the case of in vivo assays or therapeutic treatment, or in the case of in vitro assays, under conditions in which the assays are performed. In certain embodiments, non-target sequences differ from corresponding target sequences by at least 5 nucleotides.

When used as therapeutics, a provided ds oligonucleotide is administered as a pharmaceutical composition. In certain embodiments, the pharmaceutical composition comprises a therapeutically effective amount of a provided oligonucleotide comprising, or a pharmaceutically acceptable salt thereof, and at least one pharmaceutically acceptable inactive ingredient selected from pharmaceutically acceptable diluents, pharmaceutically acceptable excipients, and pharmaceutically acceptable carriers. In certain embodiments, the pharmaceutical composition is formulated for intravenous injection, oral administration, buccal administration, inhalation, nasal administration, topical administration, ophthalmic administration or otic administration. In further embodiments, the pharmaceutical composition is a tablet, a pill, a capsule, a liquid, an inhalant, a nasal spray solution, a suppository, a suspension, a gel, a colloid, a dispersion, a suspension, a solution, an emulsion, an ointment, a lotion, an eye drop or an ear drop.

### 10. Administration of Oligonucleotides and Compositions

Many delivery methods, regimen, etc. can be utilized in accordance with the present disclosure for administering provided ds oligonucleotides and compositions thereof (typically pharmaceutical compositions for therapeutic purposes), including various technologies known in the art.

In certain embodiments, a ds oligonucleotide composition, e.g., a dsRNAi oligonucleotide composition, is administered at a dose and/or frequency lower than that of an otherwise comparable reference ds oligonucleotide composition and has comparable or improved effects. In certain embodiments, a chirally controlled ds oligonucleotide composition is administered at a dose and/or frequency lower than that of a comparable, otherwise identical stereorandom reference ds oligonucleotide composition and with comparable or improved effects, e.g., in improving the knockdown of the target transcript.

In certain embodiments, the present disclosure recognizes that properties and activities, e.g., knockdown activity, stability, toxicity, etc. of ds oligonucleotides and compositions thereof can be modulated and optimized by chemical modifications and/or stereochemistry. In certain embodiments, the present disclosure provides methods for optimizing ds oligonucleotide properties and/or activities through chemical modifications and/or stereochemistry. In certain embodiments, the present disclosure provides ds oligonucleotides and compositions thereof with improved properties and/or activities. Without wishing to be bound by any theory, due to, e.g., their better activity, stability, delivery, distribution, toxicity, pharmacokinetic, pharmacodynamics and/or efficacy profiles, Applicant notes that provided ds oligonucleotides and compositions thereof in certain embodiments can be administered at lower dosage and/or reduced frequency to achieve comparable or better efficacy, and in certain embodiments can be administered at higher dosage and/or increased frequency to provide enhanced effects. In certain embodiments, the present disclosure provides chirally controlled ds oligonucleotides and compositions thereof, wherein the chirally controlled ds oligonucleotides and compositions thereof do not exhibit increased off-target effects relative non-chirally controlled ds oligonucleotides. Moreover, in certain embodiments, the present disclosure provides chirally controlled ds oligonucleotides and compositions thereof, wherein the chirally controlled ds oligonucleotides and compositions thereof exhibit increased Ago2 loading of guide strand relative non-chirally controlled ds oligonucleotides.

In certain embodiments, the present disclosure provides, in a method of administering a ds oligonucleotide composition comprising a plurality of ds oligonucleotides sharing a common base sequence, the improvement comprising administering a ds oligonucleotide comprising a plurality of ds oligonucleotides that is characterized by improved delivery relative to a reference ds oligonucleotide composition of the same common base sequence.

In certain embodiments, provided ds oligonucleotides, compositions and methods provide improved delivery. In certain embodiments, provided ds oligonucleotides, compositions and methods provide improved cytoplasmatic delivery. In certain embodiments, improved delivery is to a population of cells. In certain embodiments, improved delivery is to a tissue. In certain embodiments, improved delivery is to an organ. In certain embodiments, improved delivery is to an organism, e.g., a patient or subject. Example structural elements (e.g., chemical modifications, stereochemistry, combinations thereof, etc.), oligonucleotides, compositions and methods that provide improved delivery are extensively described in the present disclosure.

Various dosing regimens can be utilized to administer ds oligonucleotides and compositions of the present disclosure. In certain embodiments, multiple unit doses are administered, separated by periods of time. In certain embodiments, the present disclosure provides chirally controlled ds oligonucleotides and compositions thereof, wherein the chirally controlled ds oligonucleotides and compositions thereof do not exhibit diminished attributes relative non-chirally controlled ds oligonucleotides upon repeated dosing. For example, but not by way of limitation, such attributes can comprise one or more markers of liver function. Exemplary, markers of liver function include, but are not limited to ALT, alanine transaminase; AST, aspartate transaminase; ALP, alkaline phosphatase; ALB, albumin; TP, total protein. In certain embodiments, a given composition has a recommended dosing regimen, which may involve one or more doses. In certain embodiments, a dosing regimen comprises a plurality of doses each of which are separated from one another by a time period of the same length; in certain embodiments, a dosing regimen comprises a plurality of doses and at least two different time periods separating individual doses. In certain embodiments, all doses within a dosing regimen are of the same unit dose amount. In certain embodiments, different doses within a dosing regimen are of different amounts. In certain embodiments, a dosing regimen comprises a first dose in a first dose amount, followed by one or more additional doses in a second dose amount different from the first dose amount. In certain embodiments, a dosing regimen comprises a first dose in a first dose amount, followed by one or more additional doses in a second (or subsequent) dose amount that is the same as or different from the first dose (or another prior dose) amount. In certain embodiments, a chirally controlled ds oligonucleotide composition is administered according to a dosing regimen that differs from that utilized for a non-chirally controlled (e.g., stereorandom) ds oligonucleotide composition of the same sequence, and/or of a different chirally controlled ds oligonucleotide composition of the same sequence. In certain embodiments, a chirally controlled ds oligonucleotide composition is administered according to a dosing regimen that is reduced as compared with that of a chirally uncontrolled (e.g., stereorandom) ds oligonucleotide composition of the same sequence in that it achieves a lower level of total exposure over a given unit of time, involves one or more lower unit doses, and/or includes a smaller number of doses over a given unit of time. In certain embodiments, a chirally uncontrolled ds oligonucleotide is administered according to a dosing regimen that extends for a longer period of time than does that of a chirally uncontrolled (e.g., stereorandom) ds oligonucleotide composition of the same sequence. Without wishing to be limited by theory, Applicant notes that in certain embodiments, the shorter dosing regimen, and/or longer time periods between doses, may be due to the improved stability, bioavailability, and/or efficacy of a chirally controlled ds oligonucleotide composition. In certain embodiments, with their improved delivery (and other properties), provided compositions can be administered in lower dosages and/or with lower frequency to achieve biological effects, for example, clinical efficacy.

### 11. Pharmaceutical Compositions

When used as therapeutics, a provided ds oligonucleotide, e.g., a dsRNAi oligonucleotide, or ds oligonucleotide composition thereof is typically administered as a pharmaceutical composition. In certain embodiments, the present disclosure provides pharmaceutical compositions comprising a provided compound, e.g., a ds oligonucleotide, or a pharmaceutically acceptable salt thereof, and a pharmaceutical carrier. In certain embodiments, for therapeutic and clinical purposes, ds oligonucleotides of the present disclosure are provided as pharmaceutical compositions. As appreciated by those skilled in the art, ds oligonucleotides of the present disclosure can be provided in their acid, base or salt forms. In certain embodiments, ds oligonucleotides can be in acid forms, e.g., for natural phosphate linkages, in the form of —OP(O)(OH)O—; for phosphorothioate internucleotidic linkages, in the form of —OP(O)(SH)O—; etc. In certain embodiments, dsRNAi oligonucleotides can be in salt forms, e.g., for natural phosphate linkages, in the form of —OP(O)(ONa)O— in sodium salts; for phosphorothioate internucleotidic linkages, in the form of —OP(O)(SNa)O— in sodium salts; etc. Unless otherwise noted, ds oligonucleotides of the present disclosure can exist in acid, base and/or salt forms.

In certain embodiments, a pharmaceutical composition is a liquid composition. In certain embodiments, a pharmaceutical composition is provided by dissolving a solid ds oligonucleotide composition, or diluting a concentrated ds oligonucleotide composition, using a suitable solvent, e.g., water or a pharmaceutically acceptable buffer. In certain embodiments, liquid compositions comprise anionic forms of provided ds oligonucleotides and one or more cations. In certain embodiments, liquid compositions have pH values in the weak acidic, about neutral, or basic range. In certain embodiments, pH of a liquid composition is about a physiological pH, e.g., about 7.4.

In certain embodiments, a provided ds oligonucleotide is formulated for administration to and/or contact with a body cell and/or tissue expressing its target. For example, in certain embodiments, a provided dsRNAi oligonucleotide is formulated for administration to a body cell and/or tissue. In certain embodiments such a body cell and/or tissue is selected from the group consisting of: immune cells, blood cells, cardiac cells, lung cells, muscle cells, optic cells, liver cells, kidney cells, brain cells, cells of the central nervous system, and cells of the peripheral nervous system. In certain embodiments, such a body cell and/or tissue are a neuron or a cell and/or tissue of the liver. In certain embodiments, broad distribution of ds oligonucleotides and compositions may be achieved with intraparenchymal administration, intrathecal administration, or intracerebroventricular administration. In certain embodiments, the pharmaceutical composition is formulated for intravenous injection, oral administration, buccal administration, inhalation, nasal administration, topical administration, ophthalmic administration or optic administration. In certain embodiments, the pharmaceutical composition is a tablet, a pill, a capsule, a liquid, an inhalant, a nasal spray solution, a suppository, a suspension, a gel, a colloid, a dispersion, a suspension, a solution, an emulsion, an ointment, a lotion, an eye drop or an ear drop.

In certain embodiments, the present disclosure provides a pharmaceutical composition comprising chirally controlled ds oligonucleotide or composition thereof, in admixture with a pharmaceutically acceptable inactive ingredient (e.g., a pharmaceutically acceptable excipient, a pharmaceutically acceptable carrier, etc.). One of skill in the art will recognize that the pharmaceutical compositions include pharmaceutically acceptable salts of provided ds oligonucleotide or compositions. In certain embodiments, a pharmaceutical composition is a chirally controlled ds oligonucleotide composition. In certain embodiments, a pharmaceutical composition is a stereopure ds oligonucleotide composition.

In certain embodiments, the present disclosure provides salts of ds oligonucleotides and pharmaceutical compositions thereof. In certain embodiments, a salt is a pharmaceutically acceptable salt. In certain embodiments, a pharmaceutical composition comprises a ds oligonucleotide, optionally in its salt form, and a sodium salt. In certain embodiments, a pharmaceutical composition comprises a ds oligonucleotide, optionally in its salt form, and sodium chloride. In certain embodiments, each hydrogen ion of a ds oligonucleotide that may be donated to a base (e.g., under conditions of an aqueous solution, a pharmaceutical composition, etc.) is replaced by a non-H⁺ cation. For example, in certain embodiments, a pharmaceutically acceptable salt of a ds oligonucleotide is an all-metal ion salt, wherein each hydrogen ion (for example, of —OH, —SH, etc.) of each internucleotidic linkage (e.g., a natural phosphate linkage, a phosphorothioate internucleotidic linkage, etc.) is replaced by a metal ion. Various suitable metal salts for pharmaceutical compositions are widely known in the art and can be utilized in accordance with the present disclosure. In certain embodiments, a pharmaceutically acceptable salt is a sodium salt. In certain embodiments, a pharmaceutically acceptable salt is magnesium salt. In certain embodiments, a pharmaceutically acceptable salt is a calcium salt. In certain embodiments, a pharmaceutically acceptable salt is a potassium salt. In certain embodiments, a pharmaceutically acceptable salt is an ammonium salt (cation N(R)₄⁺). In certain embodiments, a pharmaceutically acceptable salt comprises one and no more than one types of cation. In certain embodiments, a pharmaceutically acceptable salt comprises two or more types of cation. In certain embodiments, a cation is Li⁺, Na⁺, K⁺, Mg²⁺ or Ca²⁺. In certain embodiments, a pharmaceutically acceptable salt is an all-sodium salt. In certain embodiments, a pharmaceutically acceptable salt is an all-sodium salt, wherein each internucleotidic linkage which is a natural phosphate linkage (acid form —O—P(O)(OH)—O—), if any, exists as its sodium salt form (—O—P(O)(ONa)—O—), and each internucleotidic linkage which is a phosphorothioate internucleotidic linkage (acid form —O—P(O)(SH)—O—), if any, exists as its sodium salt form (—O—P(O)(SNa)—O—).

Various technologies for delivering nucleic acids and/or oligonucleotides are known in the art can be utilized in accordance with the present disclosure. For example, a variety of supramolecular nanocarriers can be used to deliver nucleic acids. Example nanocarriers include, but are not limited to liposomes, cationic polymer complexes and various polymeric compounds. Complexation of nucleic acids with various polycations is another approach for intracellular delivery; this includes use of PEGylated polycations, polyethyleneamine (PEI) complexes, cationic block co-polymers, and dendrimers. Several cationic nanocarriers, including PEI and polyamidoamine dendrimers help to release contents from endosomes. Other approaches include use of polymeric nanoparticles, microspheres, liposomes, dendrimers, biodegradable polymers, conjugates, prodrugs, inorganic colloids such as sulfur or iron, antibodies, implants, biodegradable implants, biodegradable microspheres, osmotically controlled implants, lipid nanoparticles, emulsions, oily solutions, aqueous solutions, biodegradable polymers, poly(lactide-coglycolic acid), poly(lactic acid), liquid depot, polymer micelles, quantum dots and lipoplexes. In certain embodiments, a ds oligonucleotide is conjugated to another molecule.

In therapeutic and/or diagnostic applications, compounds, e.g., ds oligonucleotides, of the disclosure can be formulated for a variety of modes of administration, including systemic and topical or localized administration. Techniques and formulations generally may be found in Remington, The Science and Practice of Pharmacy (20th ed. 2000).

Pharmaceutically acceptable salts for basic moieties are generally well known to those of ordinary skill in the art, and may include, e.g., acetate, benzenesulfonate, besylate, benzoate, bicarbonate, bitartrate, bromide, calcium edetate, camsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington, The Science and Practice of Pharmacy (20th ed. 2000). Preferred pharmaceutically acceptable salts include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate.

In certain embodiments, dsRNAi oligonucleotides are formulated in pharmaceutical compositions described in WO 2005/060697, WO 2011/076807 or WO 2014/136086.

Depending on the specific conditions, disorders or diseases being treated, provided agents, e.g., ds oligonucleotides, may be formulated into liquid or solid dosage forms and administered systemically or locally. Provided ds oligonucleotides may be delivered, for example, in a timed- or sustained-low release form as is known to those skilled in the art. Techniques for formulation and administration may be found in Remington, The Science and Practice of Pharmacy (20th ed. 2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or another mode of delivery. For injection, provided agents, e.g., oligonucleotides may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulations. Such penetrants are generally known in the art and can be utilized in accordance with the present disclosure.

Use of pharmaceutically acceptable carriers to formulate compounds, e.g., provided ds oligonucleotides, for the practice of the disclosure into dosages suitable for various mods of administration is well known in the art. With proper choice of carrier and suitable manufacturing practice, compositions of the present disclosure, e.g., those formulated as solutions, may be administered via various routes, e.g., parenterally, such as by intravenous injection.

In certain embodiments, a composition comprising a dsRNAi oligonucleotide further comprises any or all of calcium chloride dihydrate, magnesium chloride hexahydrate, potassium chloride, sodium chloride, sodium phosphate dibasic anhydrous, sodium phosphate, monobasic dihydrate, and/or water for Injection. In certain embodiments, a composition further comprises any or all of calcium chloride dihydrate (0.21 mg) USP, magnesium chloride hexahydrate (0.16 mg) USP, potassium chloride (0.22 mg) USP, sodium chloride (8.77 mg) USP, sodium phosphate dibasic anhydrous (0.10 mg) USP, sodium phosphate monobasic dihydrate (0.05 mg) USP, and Water for Injection USP.

In certain embodiments, a composition comprising a ds oligonucleotide further comprises any or all of cholesterol, (6Z,9Z,28Z,31Z)-heptatriaconta-6,9,28,31-tetraen-19-yl-4-(dimethylamino) butanoate(DLin− MC3-DMA), 1,2-distearoyl-sn-glycero-3-phosphocholine (DSPC), alpha-(3′-{[1,2-di(myristyloxy)propanoxy]carbonylamino}propyl)-omega-methoxy, polyoxyethylene(PEG2000-C-DMG), potassium phosphate monobasic anhydrous NF, sodium chloride, sodium phosphate dibasic heptahydrate, and Water for Injection. In certain embodiments, the pH of a composition comprising a RNAi oligonucleotide is ˜7.0. In certain embodiments, a composition comprising an oligonucleotide further comprises any or all of: 6.2 mg cholesterol USP, 13.0 mg (6Z,9Z,28Z,31Z)-heptatriaconta-6,9,28,31-tetraen-19-yl-4-(dimethylamino) butanoate(DLin− MC3-DMA), 3.3 mg 1,2-distearoyl-sn-glycero-3-phosphocholine (DSPC), 1.6 mg α-(3′-{[1,2-di(myristyloxy)propanoxy] carbonylamino}propyl)-ω-methoxy, poly oxy ethylene(PEG2000-C-DMG), 0.2 mg potassium phosphate monobasic anhydrous NF, 8.8 mg sodium chloride USP, 2.3 mg sodium phosphate dibasic heptahydrate USP, and Water for Injection USP, in an approximately 1 mL total volume.

Provided compounds, e.g., ds oligonucleotides, can be formulated readily using pharmaceutically acceptable carriers well known in the art into dosages suitable for oral administration. In certain embodiments, such carriers enable provided oligonucleotides to be formulated as tablets, pills, capsules, liquids, gels, syrups, slurries, suspensions and the like, for, e.g., oral ingestion by a subject (e.g., patient) to be treated.

For nasal or inhalation delivery, provided compounds, e.g., ds oligonucleotides, may be formulated by methods known to those of skill in the art, and may include, e.g., examples of solubilizing, diluting, or dispersing substances such as saline, preservatives, such as benzyl alcohol, absorption promoters, and fluorocarbons.

In certain embodiments, methods of specifically localizing provided compounds, e.g., ds oligonucleotides, such as by bolus injection, may decrease median effective concentration (EC50) by a factor of 20, 25, 30, 35, 40, 45 or 50. In certain embodiments, a targeted tissue is brain tissue. In certain embodiments, a targeted tissue is striatal tissue. In certain embodiments, decreasing EC50 is desirable because it reduces the dose required to achieve a pharmacological result in a patient in need thereof.

In certain embodiments, a provided ds oligonucleotide is delivered by injection or infusion once every month, every two months, every 90 days, every 3 months, every 6 months, twice a year or once a year.

Pharmaceutical compositions suitable for use in the present disclosure include compositions wherein the active ingredients, e.g., ds oligonucleotides, are contained in effective amounts to achieve their intended purposes. Determination of the effective amounts is well within the capability of those skilled in the art, especially in light of the detailed disclosure provided herein.

In addition to active ingredients, pharmaceutical compositions may contain suitable pharmaceutically acceptable carriers comprising excipients and auxiliaries which facilitate processing of an active compound into preparations which can be used pharmaceutically. Preparations formulated for oral administration may be in the form of tablets, dragees, capsules, or solutions.

In certain embodiments, pharmaceutical compositions for oral use can be obtained by combining an active compound with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

In certain embodiments, dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. Push-fit capsules can contain active ingredients, e.g., ds oligonucleotides, in admixture with fillers such as lactose, binders such as starches, and/or lubricants such as talc or magnesium stearate and, optionally, stabilizers. In soft capsules, active compounds, e.g., ds oligonucleotides, may be dissolved or suspended in suitable liquids, such as fatty oils, liquid paraffin, or liquid polyethylene glycols (PEGs). In addition, stabilizers may be added.

In certain embodiments, a provided composition comprises a lipid. In certain embodiments, a lipid is conjugated to an active compound, e.g., an oligonucleotide. In certain embodiments, a lipid is not conjugated to an active compound. In certain embodiments, a lipid comprises a C₁₀-C₄₀ linear, saturated or partially unsaturated, aliphatic chain. In certain embodiments, a lipid comprises a C₁₀-C₄₀ linear, saturated or partially unsaturated, aliphatic chain, optionally substituted with one or more C₁₄ aliphatic group. In certain embodiments, the lipid is selected from the group consisting of lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl alcohol. In certain embodiments, an active compound is a provided oligonucleotide. In certain embodiments, a composition comprises a lipid and an an active compound, and further comprises another component which is another lipid or a targeting compound or moiety. In certain embodiments, a lipid is an amino lipid; an amphipathic lipid; an anionic lipid; an apolipoprotein; a cationic lipid; a low molecular weight cationic lipid; a cationic lipid such as CLinDMA and DLinDMA; an ionizable cationic lipid; a cloaking component; a helper lipid; a lipopeptide; a neutral lipid; a neutral zwitterionic lipid; a hydrophobic small molecule; a hydrophobic vitamin; a PEG-lipid; an uncharged lipid modified with one or more hydrophilic polymers; phospholipid; a phospholipid such as 1,2-dioleoyl-sn-glycero-3-phosphoethanolamine; a stealth lipid; a sterol; a cholesterol; a targeting lipid; or another lipid described herein or reported in the art suitable for pharmaceutical uses. In certain embodiments, a composition comprises a lipid and a portion of another lipid capable of mediating at least one function of another lipid. In certain embodiments, a targeting compound or moiety is capable of targeting a compound (e.g., a ds oligonucleotide) to a particular cell or tissue or subset of cells or tissues. In certain embodiments, a targeting moiety is designed to take advantage of cell- or tissue-specific expression of particular targets, receptors, proteins, or another subcellular component. In certain embodiments, a targeting moiety is a ligand (e.g., a small molecule, antibody, peptide, protein, carbohydrate, aptamer, etc.) that targets a composition to a cell or tissue, and/or binds to a target, receptor, protein, or another subcellular component.

Certain example lipids for delivery of an active compound, e.g., a ds oligonucleotide, allow (e.g., do not prevent or interfere with) the function of an active compound. In certain embodiments, a lipid is lauric acid, myristic acid, palmitic acid, stearic acid, oleic acid, linoleic acid, alpha-linolenic acid, gamma-linolenic acid, docosahexaenoic acid (cis-DHA), turbinaric acid or dilinoleyl alcohol.

As described in the present disclosure, lipid conjugation, such as conjugation with fatty acids, may improve one or more properties of ds oligonucleotides.

In certain embodiments, a composition for delivery of an active compound, e.g., a ds oligonucleotide, is capable of targeting an active compound to particular cells or tissues as desired. In certain embodiments, a composition for delivery of an active compound is capable of targeting an active compound to a muscle cell or tissue. In certain embodiments, the present disclosure provides compositions and methods related to delivery of active compounds, wherein the compositions comprise an active compound and a lipid. In various embodiments to a hepatic cell or tissue, a lipid is selected from lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl alcohol.

In certain embodiments, a dsRNAi oligonucleotide is delivered to the central nervous or hepetic system, or a cell or tissue or portion thereof, via a delivery method or composition designed for delivery of nucleic acids to the central nervous or hepetic system, or a cell or tissue or portion thereof.

In certain embodiments, a dsRNAi oligonucleotide is delivered via a composition comprising any one or more of, or a method of delivery involving the use of any one or more of: transferrin receptor-targeted nanoparticle; cationic liposome-based delivery strategy; cationic liposome; polymeric nanoparticle; viral carrier; retrovirus; adeno-associated virus; stable nucleic acid lipid particle; polymer; cell-penetrating peptide; lipid; dendrimer; neutral lipid; cholesterol; lipid-like molecule; fusogenic lipid; hydrophilic molecule; polyethylene glycol (PEG) or a derivative thereof; shielding lipid; PEGylated lipid; PEG-C-DMSO; PEG-C-DMSA; DSPC; ionizable lipid; a guanidinium-based cholesterol derivative; ion-coated nanoparticle; metal-ion coated nanoparticle; manganese ion-coated nanoparticle; angubindin-1; nanogel; incorporation of the dsRNAi into a branched nucleic acid structure; and/or incorporation of the dsRNAi into a branched nucleic acid structure comprising 2, 3, 4 or more oligonucleotides.

In certain embodiments, a composition comprising a ds oligonucleotide is lyophilized. In certain embodiments, a composition comprising a ds oligonucleotide is lyophilized, and the lyophilized ds oligonucleotide is in a vial. In certain embodiments, the vial is back filled with nitrogen. In certain embodiments, the lyophilized ds oligonucleotide composition is reconstituted prior to administration. In certain embodiments, the lyophilized ds oligonucleotide composition is reconstituted with a sodium chloride solution prior to administration. In certain embodiments, the lyophilized ds oligonucleotide composition is reconstituted with a 0.9% sodium chloride solution prior to administration. In certain embodiments, reconstitution occurs at the clinical site for administration. In certain embodiments, in a lyophilized composition, a ds oligonucleotide composition is chirally controlled or comprises at least one chirally controlled internucleotidic linkage and/or the ds oligonucleotide targets.

### II. EXEMPLIFICATION

Various technologies can be utilized to assess properties and/or activities of provided oligonucleotides and compositions thereof. Some such technologies are described in this Example. Those skilled in the art appreciate that many other technologies can be readily utilized. As demonstrated herein, provided oligonucleotides and compositions, among other things, can be highly active, e.g., in reducing levels of their target nucleic acids.

Certain examples of provided technologies (compounds (oligonucleotides, reagents, etc.), compositions, methods (methods of preparation, use, assessment, etc.), etc.) were presented herein.

### Example 1. Oligonucleotide Synthesis

Various technologies for preparing oligonucleotides and oligonucleotide compositions (both stereorandom and chirally controlled) are known and can be utilized in accordance with the present disclosure, including, for example, those in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, WO 2020/191252, and/or WO 2021/071858, the methods and reagents of each of which are incorporated herein by reference. Stereorandom and chirally controlled guide strand sequences were prepared utilizing the synthetic procedures as exemplified in above mentioned disclosures. Respective passenger strands were designed to have covalently linked GalNAc moiety as delivery vehicle at either end of sequences. Oligonucleotides with 5′-GalNAc modifications were synthesized by coupling C6-amino modifier linker at the 5′-end of sequence. Oligonucleotides with 3′-GalNAc moiety as delivery vehicle were synthesized by utilizing 3′-C6 amino modified support. The single strand was cleaved from CPG by using deprotection condition as exemplified in earlier disclosures. The resulting amino group containing crude oligonucleotide was purified by ion exchange chromatography on AKTA pure system using a sodium chloride gradient. Desired product was desalted and further used for conjugation with GalNAc acid. After conjugation reaction was found to be complete the material was further purified by ion exchange chromatography and desalted to achieve desired material. For introduction of PN linkages in guide and passenger strands, specific PN coupling cycles were introduced at desired positions in oligonucleotide sequence utilizing the conditions as exemplified in WO2019/200185.

In certain embodiments, oligonucleotides were prepared using suitable chiral auxiliaries, e.g., DPSE and PSM chiral auxiliaries. Various oligonucleotides, e.g., those in Table 1, and compositions thereof, were prepared in accordance with the present disclosure.

Various technologies can be utilized to assess properties and/or activities of provided oligonucleotides and compositions thereof. Some such technologies are described in this Example. Those skilled in the art appreciate that many other technologies can be readily utilized. As demonstrated herein, provided oligonucleotides and compositions, among other things, can be highly active, e.g., in reducing levels of their target nucleic acids.

### Example 2. Provided Oligonucleotides and Compositions can Effectively Knockdown Mouse Transthyretin (mTTR) In Vitro

Various siRNAs for mouse TTR were designed and constructed. A number of siRNAs were tested in vitro in mouse primary hepatocytes at one or a range of concentrations. Some siRNAs were also tested in mice (e.g., C57BL6 wild type mice).

Example protocol for in vitro determination of siRNA activity: For determination of siRNAs activity, siRNAs at specific concentration were gymnotically delivered to mouse primary hepatocytes plated at 96-well plates, with 10,000 cells/well. Following 48 hours treatment, total RNA was extracted using SV96 Total RNA Isolation kit (Promega). cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). For mouse TTR mRNA, the following qPCR assay were utilized: IDT Tagman qPCR assay ID Mm.PT.58.11922308. Mouse HPRT was used as normalizer (Forward 5′CAAACTTTGCTTTCCCTGGTT3′, Reverse 5′TGGCCTGTATCCAACACTTC3′, Probe 5′/5HEX/ACCAGCAAG/Zen/CTTGCAACCTTAACC/3IABkFQ/3′. mRNA knockdown levels were calculated as % mRNA remaining relative to mock treatment.

Table 2 shows % mouse TTR mRNA remaining (at 300 and 100 pM siRNA treatment) relative to mouse HPRT control. N=2. N.D.: Not determined

TABLE 2

| | | 300 pM | | | 100 pM | | |
|---|---|---|---|---|---|---|---|
| | | % remaining | % remaining | | % remaining | % remaining | |
| | | mRNA | mRNA | | mRNA | mRNA | |
| | | (mTTR/ | (mTTR/ | | (mTTR/ | (mTTR/ | |
| Guide | Passenger | mHPRT)-1 | mHPRT)-2 | Mean | mHPRT)-1 | mHPRT)-2 | Mean |
| WV- | WV-41828 | 20.97 | 9.55 | 15.26 | 28.39 | 28.31 | 28.35 |
| 41826 | | | | | | | |
| WV- | WV-42080 | 14.68 | 7.96 | 11.32 | 34.42 | 21.61 | 28.02 |
| 43774 | | | | | | | |
| WV- | WV-42080 | 70.03 | 39.56 | 54.79 | 66.39 | 79.40 | 72.89 |
| 46497 | | | | | | | |
| WV- | WV-42080 | 76.91 | 46.39 | 61.65 | 81.63 | 96.42 | 89.03 |
| 46498 | | | | | | | |
| WV- | WV-42080 | 91.21 | 55.23 | 73.22 | 69.53 | 82.31 | 75.92 |
| 46499 | | | | | | | |
| WV- | WV-42080 | 50.21 | 38.60 | 44.40 | 52.59 | 69.57 | 61.08 |
| 46500 | | | | | | | |
| WV- | WV-42080 | 66.94 | 41.92 | 54.43 | 62.10 | 73.46 | 67.78 |
| 46501 | | | | | | | |
| WV- | WV-42080 | 59.20 | 26.87 | 43.03 | 47.51 | 57.88 | 52.70 |
| 46502 | | | | | | | |
| WV- | WV-42080 | 38.11 | 18.54 | 28.32 | 50.09 | 54.96 | 52.53 |
| 46503 | | | | | | | |
| WV- | WV-42080 | 25.74 | 20.31 | 23.03 | 41.53 | 54.86 | 48.19 |
| 46504 | | | | | | | |
| WV- | WV-42080 | 22.38 | 12.31 | 17.35 | 33.18 | 35.23 | 34.21 |
| 46505 | | | | | | | |
| WV- | WV-42080 | 18.00 | 7.05 | 12.53 | 24.52 | 21.39 | 22.96 |
| 46506 | | | | | | | |
| WV- | WV-42080 | 19.28 | 8.28 | 13.78 | 23.82 | 18.67 | 21.24 |
| 46507 | | | | | | | |
| WV- | WV-42080 | 14.82 | 8.42 | 11.62 | 20.67 | 20.28 | 20.48 |
| 46508 | | | | | | | |
| WV- | WV-42080 | 14.85 | 5.52 | 10.18 | 14.93 | 18.69 | 16.81 |
| 46509 | | | | | | | |
| WV- | WV-42080 | 17.38 | 6.71 | 12.05 | 23.29 | 26.13 | 24.71 |
| 46510 | | | | | | | |
| WV- | WV-42080 | 25.37 | 16.09 | 20.73 | 29.22 | 26.76 | 27.99 |
| 46511 | | | | | | | |
| WV- | WV-42080 | 18.98 | 9.61 | 14.29 | 28.86 | 21.48 | 25.17 |
| 46512 | | | | | | | |
| WV- | WV-42080 | 18.95 | 7.11 | 13.03 | 23.65 | 23.34 | 23.50 |
| 46513 | | | | | | | |
| WV- | WV-42080 | 16.64 | 9.88 | 13.26 | 26.03 | 21.27 | 23.65 |
| 46514 | | | | | | | |
| WV- | WV-42080 | 18.62 | 10.15 | 14.39 | 20.35 | 20.33 | 20.34 |
| 46515 | | | | | | | |
| WV- | WV-42080 | 13.69 | 7.60 | 10.65 | 22.03 | 29.38 | 25.71 |
| 46516 | | | | | | | |
| WV- | WV-42080 | 19.79 | 8.80 | 14.30 | 21.31 | 32.56 | 26.93 |
| 46517 | | | | | | | |
| WV- | WV-42080 | 34.78 | 18.71 | 26.74 | 35.96 | 62.86 | 49.41 |
| 46518 | | | | | | | |
| WV- | WV-42080 | 86.53 | 80.02 | 83.28 | 81.69 | 116.95 | 99.32 |
| 46519 | | | | | | | |
| WV- | WV-42080 | 20.90 | 14.31 | 17.60 | 35.17 | 35.18 | 35.17 |
| 46520 | | | | | | | |
| WV- | WV-42080 | 17.35 | 6.75 | 12.05 | 18.42 | 26.07 | 22.24 |
| 45148 | | | | | | | |
| WV- | WV-42080 | 19.29 | 13.21 | 16.25 | 32.78 | 25.54 | 29.16 |
| 46521 | | | | | | | |
| WV- | WV-42080 | 19.24 | 12.31 | 15.77 | 31.89 | 22.30 | 27.09 |
| 46522 | | | | | | | |
| WV- | WV-42080 | 25.12 | 11.76 | 18.44 | 52.29 | 39.47 | 45.88 |
| 46523 | | | | | | | |
| WV- | WV-42080 | 21.13 | 9.38 | 15.25 | 27.02 | 32.73 | 29.88 |
| 46524 | | | | | | | |
| WV- | WV-42080 | 18.08 | 10.96 | 14.52 | 29.15 | 28.91 | 29.03 |
| 46525 | | | | | | | |
| WV- | WV-42080 | 34.55 | 22.04 | 28.29 | 73.34 | 44.96 | 59.15 |
| 46526 | | | | | | | |
| WV- | WV-42080 | 17.14 | 11.23 | 14.18 | 49.05 | 36.29 | 42.67 |
| 45147 | | | | | | | |
| WV- | WV-42080 | 16.85 | 8.32 | 12.58 | 33.72 | 30.59 | 32.16 |
| 46527 | | | | | | | |
| WV- | WV-42080 | 13.88 | 9.17 | 11.53 | 45.44 | 20.60 | 33.02 |
| 46528 | | | | | | | |
| WV- | WV-42080 | 21.65 | 9.79 | 15.72 | 46.40 | 22.60 | 34.50 |
| 46529 | | | | | | | |
| WV- | WV-42080 | 13.80 | 5.68 | 9.74 | 34.20 | 22.42 | 28.31 |
| 46530 | | | | | | | |
| WV- | WV-42080 | 15.66 | 6.02 | 10.84 | 38.57 | 22.77 | 30.67 |
| 46531 | | | | | | | |
| WV- | WV-42080 | 13.28 | 8.95 | 11.12 | 25.40 | 28.54 | 26.97 |
| 46532 | | | | | | | |
| WV- | WV-42080 | 28.49 | 13.60 | 21.05 | 68.07 | 32.38 | 50.23 |
| 46533 | | | | | | | |
| WV- | WV-42080 | 19.19 | 11.80 | 15.49 | 70.21 | 51.48 | 60.84 |
| 46534 | | | | | | | |
| WV- | WV-42080 | 19.39 | 8.82 | 14.10 | 50.18 | 27.87 | 39.03 |
| 45146 | | | | | | | |
| WV- | WV-42080 | 19.48 | 12.42 | 15.95 | 57.91 | 29.34 | 43.62 |
| 46535 | | | | | | | |
| WV- | WV-42080 | 28.11 | 21.20 | 24.65 | 50.05 | 33.47 | 41.76 |
| 46536 | | | | | | | |
| WV- | WV-42080 | 40.51 | 22.98 | 31.74 | 75.21 | 72.61 | 73.91 |
| 46537 | | | | | | | |
| WV- | WV-42080 | 12.77 | 5.13 | 8.95 | 45.51 | 16.53 | 31.02 |
| 43775 | | | | | | | |
| WV- | WV-42080 | 35.23 | 39.33 | 37.28 | 62.87 | 74.35 | 68.61 |
| 46538 | | | | | | | |
| WV- | WV-42080 | 64.93 | 55.56 | 60.24 | 104.62 | 91.69 | 98.16 |
| 46539 | | | | | | | |
| WV- | WV-42080 | 95.13 | 92.02 | 93.57 | 118.58 | 170.68 | 144.63 |
| 46540 | | | | | | | |
| WV- | WV-42080 | 93.84 | 91.20 | 92.52 | 106.06 | 133.38 | 119.72 |
| 46541 | | | | | | | |
| WV- | WV-42080 | 95.39 | 93.79 | 94.59 | 121.70 | 105.25 | 113.47 |
| 46542 | | | | | | | |
| WV- | WV-42080 | 79.46 | 76.17 | 77.81 | 93.78 | 89.69 | 91.73 |
| 46543 | | | | | | | |
| WV- | WV-42080 | 43.39 | 23.30 | 33.34 | 57.83 | 60.72 | 59.27 |
| 46544 | | | | | | | |
| WV- | WV-42080 | 22.79 | 14.42 | 18.61 | 54.11 | 31.43 | 42.77 |
| 46545 | | | | | | | |
| WV- | WV-42080 | 14.49 | 18.59 | 16.54 | 36.46 | 26.68 | 31.57 |
| 46546 | | | | | | | |
| WV- | WV-42080 | 28.03 | 19.12 | 23.57 | 70.19 | 39.81 | 55.00 |
| 46547 | | | | | | | |
| WV- | WV-42080 | 27.44 | 11.40 | 19.42 | 46.30 | 48.61 | 47.45 |
| 46548 | | | | | | | |
| WV- | WV-42080 | 14.37 | 13.12 | 13.75 | 45.66 | 16.55 | 31.11 |
| 46549 | | | | | | | |
| WV- | WV-42080 | 14.48 | 12.54 | 13.51 | 44.85 | 23.04 | 33.95 |
| 46550 | | | | | | | |
| WV- | WV-42080 | 16.88 | 12.66 | 14.77 | 42.12 | 23.64 | 32.88 |
| 46551 | | | | | | | |
| WV- | WV-42080 | 15.74 | 7.92 | 11.83 | 39.08 | 15.85 | 27.47 |
| 46552 | | | | | | | |
| WV- | WV-42080 | 12.78 | 7.91 | 10.34 | 45.57 | 13.76 | 29.66 |
| 46553 | | | | | | | |
| WV- | WV-42080 | 11.70 | 15.19 | 13.44 | 36.89 | 23.50 | 30.20 |
| 46554 | | | | | | | |
| WV- | WV-42080 | 26.08 | 16.81 | 21.44 | 69.89 | 42.07 | 55.98 |
| 46555 | | | | | | | |
| WV- | WV-42080 | 16.49 | 15.02 | 15.75 | 55.15 | 42.40 | 48.77 |
| 46556 | | | | | | | |
| WV- | WV-42080 | 17.85 | 13.55 | 15.70 | 52.45 | 18.07 | 35.26 |
| 46557 | | | | | | | |
| WV- | WV-42080 | 17.26 | 13.33 | 15.30 | 69.51 | 31.96 | 50.73 |
| 46558 | | | | | | | |
| WV- | WV-42080 | 61.98 | 55.69 | 58.84 | 112.54 | 90.83 | 101.69 |
| 46559 | | | | | | | |
| WV- | WV-42080 | 62.38 | 47.04 | 54.71 | 126.87 | 87.33 | 107.10 |
| 46560 | | | | | | | |
| WV- | WV-42080 | 17.76 | 8.67 | 13.21 | 56.51 | 19.25 | 37.88 |
| 46561 | | | | | | | |
| WV- | WV-42080 | 13.95 | 8.78 | 11.37 | 42.12 | 24.66 | 33.39 |
| 44453 | | | | | | | |
| WV- | WV-42080 | 29.49 | 26.17 | 27.83 | 69.04 | 48.26 | 58.65 |
| 46562 | | | | | | | |
| WV- | WV-42080 | 16.61 | 16.55 | 16.58 | 63.78 | 71.29 | 67.54 |
| 46563 | | | | | | | |
| WV- | WV-42080 | 35.65 | 22.52 | 29.08 | 68.01 | 27.69 | 47.85 |
| 46564 | | | | | | | |
| WV- | WV-42080 | 13.53 | 12.49 | 13.01 | 55.93 | 22.05 | 38.99 |
| 46565 | | | | | | | |
| WV- | WV-42080 | 18.91 | 11.98 | 15.44 | 46.07 | 35.93 | 41.00 |
| 46566 | | | | | | | |
| WV- | WV-42080 | 22.86 | 8.87 | 15.86 | 53.19 | 41.64 | 47.42 |
| 46567 | | | | | | | |
| WV- | WV-42080 | 12.97 | 5.56 | 9.26 | 48.84 | 9.81 | 29.32 |
| 44452 | | | | | | | |
| WV- | WV-42080 | 11.60 | 6.52 | 9.06 | 35.53 | 12.31 | 23.92 |
| 46568 | | | | | | | |
| WV- | WV-42080 | 23.84 | 17.79 | 20.81 | 71.40 | 30.94 | 51.17 |
| 46569 | | | | | | | |
| WV- | WV-42080 | 14.13 | 17.70 | 15.91 | 50.77 | 41.49 | 46.13 |
| 46570 | | | | | | | |
| WV- | WV-42080 | 13.19 | 8.16 | 10.68 | 48.83 | 13.99 | 31.41 |
| 46571 | | | | | | | |
| WV- | WV-42080 | 14.22 | 7.18 | 10.70 | 48.28 | 19.49 | 33.89 |
| 46572 | | | | | | | |
| WV- | WV-42080 | 15.06 | 8.59 | 11.83 | 55.14 | 21.69 | 38.41 |
| 46573 | | | | | | | |
| WV- | WV-42080 | 14.57 | 6.56 | 10.57 | 44.47 | 26.89 | 35.68 |
| 46574 | | | | | | | |
| WV- | WV-42080 | 14.50 | 7.13 | 10.81 | 54.14 | 15.12 | 34.63 |
| 46575 | | | | | | | |
| WV- | WV-42080 | 20.85 | 9.59 | 15.22 | 46.71 | 22.19 | 34.45 |
| 44451 | | | | | | | |
| WV- | WV-42080 | 26.56 | 37.62 | 32.09 | 97.87 | 52.55 | 75.21 |
| 46576 | | | | | | | |
| WV- | WV-42080 | 26.61 | 21.64 | 24.13 | 87.10 | 42.24 | 64.67 |
| 46577 | | | | | | | |
| WV- | WV-42080 | 67.11 | 45.99 | 56.55 | 151.49 | 94.13 | 122.81 |
| 44457 | | | | | | | |

Table 3 shows % mouse TTR mRNA remaining (at 150 and 50 pM siRNA treatment) relative to mouse HPRT control. N=2. N.D.: Not determined.

TABLE 3

| | | 150 pM | | | 50 pM | | |
|---|---|---|---|---|---|---|---|
| | | % remaining | % remaining | | % remaining | % remaining | |
| | | mRNA (mTTR/ | mRNA (mTTR/ | | mRNA (mTTR/ | mRNA (mTTR/ | |
| Guide | Passenger | mHPRT) − 1 | mHPRT) − 2 | Mean | mHPRT) − 1 | mHPRT) − 2 | Mean |
| WV-41826 | WV-41828 | 22.26 | 25.20 | 23.73 | 50.05 | 40.25 | 45.15 |
| WV-43774 | WV-42080 | 5.40 | 8.77 | 7.09 | 28.95 | 20.75 | 24.85 |
| WV-46497 | WV-42080 | 82.75 | 71.33 | 77.04 | 101.23 | 83.15 | 92.19 |
| WV-47066 | WV-42080 | 61.90 | 52.60 | 57.25 | 76.31 | 77.74 | 77.02 |
| WV-47067 | WV-42080 | 64.13 | 54.81 | 59.47 | 83.94 | 74.87 | 79.41 |
| WV-47068 | WV-42080 | 29.04 | 20.26 | 24.65 | 80.52 | 44.66 | 62.59 |
| WV-46501 | WV-42080 | 90.57 | 69.35 | 79.96 | 89.23 | 72.59 | 80.91 |
| WV-47069 | WV-42080 | 57.23 | 39.87 | 48.55 | 76.67 | 59.90 | 68.29 |
| WV-47070 | WV-42080 | 21.54 | 15.76 | 18.65 | 34.41 | N.D. | 34.41 |
| WV-47071 | WV-42080 | 33.79 | 27.25 | 30.52 | 68.72 | 54.95 | 61.83 |
| WV-47072 | WV-42080 | 26.73 | 32.92 | 29.83 | 63.44 | 40.66 | 52.05 |
| WV-47073 | WV-42080 | 19.90 | 18.28 | 19.09 | 39.91 | 34.15 | 37.03 |
| WV-47074 | WV-42080 | 17.11 | 19.92 | 18.52 | 49.54 | 31.21 | 40.37 |
| WV-47075 | WV-42080 | 15.26 | 11.52 | 13.39 | 38.03 | 22.35 | 30.19 |
| WV-46509 | WV-42080 | 23.96 | 16.04 | 20.00 | 45.03 | 33.79 | 39.41 |
| WV-47076 | WV-42080 | 22.90 | 23.33 | 23.12 | 63.05 | 28.49 | 45.77 |
| WV-46511 | WV-42080 | 19.24 | 21.07 | 20.16 | 37.49 | 20.88 | 29.19 |
| WV-47077 | WV-42080 | 14.19 | 15.45 | 14.82 | 36.43 | 31.56 | 34.00 |
| WV-47078 | WV-42080 | 20.07 | 24.66 | 22.37 | 47.21 | 30.72 | 38.97 |
| WV-47079 | WV-42080 | 23.13 | 19.25 | 21.19 | 52.12 | 37.53 | 44.82 |
| WV-47080 | WV-42080 | 21.41 | 17.12 | 19.27 | 54.29 | 34.77 | 44.53 |
| WV-47081 | WV-42080 | 19.41 | 18.71 | 19.06 | 52.97 | 40.80 | 46.89 |
| WV-47082 | WV-42080 | 34.04 | 29.30 | 31.67 | 63.50 | 43.76 | 53.63 |
| WV-47083 | WV-42080 | 49.16 | 47.42 | 48.29 | 78.12 | 56.28 | 67.20 |
| WV-46519 | WV-42080 | 93.76 | 80.79 | 87.28 | 108.86 | 78.58 | 93.72 |
| WV-47084 | WV-42080 | 16.97 | 22.13 | 19.55 | 48.81 | 35.71 | 42.26 |
| WV-47085 | WV-42080 | 14.10 | 16.96 | 15.53 | 42.89 | 30.21 | 36.55 |
| WV-47086 | WV-42080 | 29.48 | 31.48 | 30.48 | 61.70 | 48.42 | 55.06 |
| WV-46522 | WV-42080 | 18.68 | 18.87 | 18.78 | 54.34 | 33.49 | 43.92 |
| WV-47087 | WV-42080 | 21.46 | 18.18 | 19.82 | 50.61 | 45.85 | 48.23 |
| WV-47088 | WV-42080 | 19.28 | 19.51 | 19.40 | 46.39 | 35.14 | 40.77 |
| WV-47089 | WV-42080 | 27.71 | 25.91 | 26.81 | 85.83 | 34.45 | 60.14 |
| WV-47090 | WV-42080 | 27.43 | 25.47 | 26.45 | 45.60 | 39.69 | 42.64 |
| WV-47091 | WV-42080 | 12.03 | 13.96 | 13.00 | 51.70 | 25.64 | 38.67 |
| WV-47092 | WV-42080 | 16.05 | 17.88 | 16.97 | 43.39 | 28.52 | 35.95 |
| WV-47093 | WV-42080 | 11.11 | 11.04 | 11.08 | 36.13 | 24.41 | 30.27 |
| WV-46529 | WV-42080 | 17.89 | 19.82 | 18.86 | 52.84 | 31.98 | 42.41 |
| WV-47094 | WV-42080 | 19.05 | 15.46 | 17.26 | 47.91 | 33.06 | 40.49 |
| WV-46531 | WV-42080 | 22.99 | 20.19 | 21.59 | 57.04 | 33.11 | 45.08 |
| WV-47095 | WV-42080 | 19.42 | 25.75 | 22.59 | 56.40 | 28.46 | 42.43 |
| WV-47096 | WV-42080 | 17.40 | 18.22 | 17.81 | 37.51 | 28.34 | 32.92 |
| WV-47097 | WV-42080 | 14.31 | 22.07 | 18.19 | 57.38 | 41.19 | 49.28 |
| WV-47098 | WV-42080 | 25.74 | 23.62 | 24.68 | 52.42 | 34.50 | 43.46 |
| WV-47099 | WV-42080 | 22.21 | 19.87 | 21.04 | 56.91 | 37.80 | 47.35 |
| WV-47100 | WV-42080 | 33.51 | 34.34 | 33.93 | 72.35 | 58.25 | 65.30 |
| WV-47101 | WV-42080 | 54.85 | 27.04 | 40.95 | 80.34 | 57.94 | 69.14 |
| WV-43775 | WV-42080 | 12.21 | 12.32 | 12.27 | 39.89 | 29.10 | 34.49 |
| WV-46538 | WV-42080 | 40.70 | 49.76 | 45.23 | 71.66 | 51.51 | 61.59 |
| WV-47102 | WV-42080 | 93.26 | 82.90 | 88.08 | 95.81 | 95.48 | 95.64 |
| WV-47103 | WV-42080 | 87.39 | 79.98 | 83.69 | 90.49 | 92.11 | 91.30 |
| WV-47104 | WV-42080 | 86.54 | 69.40 | 77.97 | 96.14 | 84.35 | 90.25 |
| WV-46542 | WV-42080 | 99.51 | 83.39 | 91.45 | 93.61 | 95.98 | 94.79 |
| WV-47105 | WV-42080 | 69.91 | 68.68 | 69.30 | 97.53 | 77.42 | 87.47 |
| WV-47106 | WV-42080 | 19.97 | 18.89 | 19.43 | 50.71 | 38.87 | 44.79 |
| WV-47107 | WV-42080 | 26.94 | 32.53 | 29.74 | 67.24 | 50.66 | 58.95 |
| WV-47108 | WV-42080 | 15.83 | 17.73 | 16.78 | 42.11 | 36.17 | 39.14 |
| WV-47109 | WV-42080 | 11.89 | 12.96 | 12.43 | 32.78 | 22.55 | 27.67 |
| WV-47110 | WV-42080 | 8.36 | 10.70 | 9.53 | 78.11 | 71.01 | 74.56 |
| WV-47111 | WV-42080 | 10.11 | 10.45 | 10.28 | 35.70 | 14.96 | 25.33 |
| WV-46550 | WV-42080 | 15.47 | 12.67 | 14.07 | 37.62 | 24.57 | 31.09 |
| WV-47112 | WV-42080 | 16.35 | 16.10 | 16.23 | 50.64 | 30.38 | 40.51 |
| WV-46552 | WV-42080 | 12.59 | 9.50 | 11.05 | 36.78 | 25.47 | 31.12 |
| WV-47113 | WV-42080 | 14.69 | 15.45 | 15.07 | 64.55 | 34.52 | 49.53 |
| WV-47114 | WV-42080 | 13.05 | 16.32 | 14.69 | 31.72 | 20.40 | 26.06 |
| WV-47115 | WV-42080 | 25.49 | 26.79 | 26.14 | 55.31 | 41.16 | 48.23 |
| WV-47116 | WV-42080 | 9.68 | 13.06 | 11.37 | 36.73 | 27.49 | 32.11 |
| WV-47117 | WV-42080 | 13.32 | 15.33 | 14.33 | 45.56 | 33.32 | 39.44 |
| WV-47118 | WV-42080 | 19.21 | 22.14 | 20.68 | 45.08 | 35.43 | 40.25 |
| WV-47119 | WV-42080 | 68.00 | 81.08 | 74.54 | 93.77 | 83.88 | 88.82 |
| WV-46560 | WV-42080 | 79.31 | 76.27 | 77.79 | 89.09 | 95.20 | 92.15 |
| WV-47120 | WV-42080 | 20.89 | 26.38 | 23.64 | 54.39 | 47.66 | 51.02 |
| WV-47121 | WV-42080 | 12.17 | 10.46 | 11.32 | 30.81 | 18.32 | 24.56 |
| WV-47122 | WV-42080 | 116.01 | 110.76 | 113.39 | 105.59 | 108.27 | 106.93 |
| WV-46563 | WV-42080 | 13.46 | 17.20 | 15.33 | 42.72 | 35.58 | 39.15 |
| WV-47123 | WV-42080 | 19.20 | 21.22 | 20.21 | 49.44 | 44.02 | 46.73 |
| WV-47124 | WV-42080 | 15.10 | 14.13 | 14.62 | 32.49 | 20.34 | 26.42 |
| WV-47125 | WV-42080 | 23.48 | 25.06 | 24.27 | 58.50 | 39.32 | 48.91 |
| WV-47126 | WV-42080 | 22.49 | 18.90 | 20.70 | 46.16 | 35.10 | 40.63 |
| WV-47127 | WV-42080 | 13.93 | 19.99 | 16.96 | 39.16 | 31.64 | 35.40 |
| WV-47128 | WV-42080 | 11.50 | 14.54 | 13.02 | 34.92 | 21.62 | 28.27 |
| WV-47129 | WV-42080 | 12.36 | 14.64 | 13.50 | 28.85 | 21.46 | 25.16 |
| WV-46570 | WV-42080 | 9.19 | 9.43 | 9.31 | 31.00 | 27.58 | 29.29 |
| WV-47130 | WV-42080 | 15.73 | 13.71 | 14.72 | 34.69 | 28.57 | 31.63 |
| WV-46572 | WV-42080 | 16.38 | 11.85 | 14.12 | 30.96 | 22.17 | 26.56 |
| WV-47131 | WV-42080 | 15.70 | 12.95 | 14.33 | 38.71 | 20.54 | 29.62 |
| WV-47132 | WV-42080 | 14.71 | 16.70 | 15.71 | 42.28 | 29.11 | 35.69 |
| WV-47133 | WV-42080 | 20.94 | 24.50 | 22.72 | 44.57 | 44.20 | 44.38 |
| WV-47134 | WV-42080 | 22.32 | 28.48 | 25.40 | 54.28 | 29.36 | 41.82 |
| WV-47135 | WV-42080 | 22.41 | 22.59 | 22.50 | 39.15 | 22.20 | 30.68 |
| WV-47136 | WV-42080 | 16.83 | 22.35 | 19.59 | N.D. | 38.36 | 38.36 |
| WV-47137 | WV-42080 | 74.92 | 66.57 | 70.75 | 93.33 | 111.71 | 102.52 |

Table 4 shows % mouse TTR mRNA remaining (at 150, 100 and 50 pM siRNA treatment) relative to mouse HPRT control. N=2. N.D.: Not determined.

TABLE 4

| | | 150 pM | | | 100 pM | | | 50 pM | | |
|---|---|---|---|---|---|---|---|---|---|---|
| | | % remaining | % remaining | | % remaining | % remaining | | % remaining | % remaining | |
| | | mRNA | mRNA | | mRNA | mRNA | | mRNA | mRNA | |
| | | (mTTR/ | (mTTR/ | | (mTTR/ | (mTTR/ | | (mTTR/ | (mTTR/ | |
| Guide | Passenger | mHPRT) − 1 | mHPRT) − 2 | Mean | mHPRT) − 1 | mHPRT) − 2 | Mean | mHPRT) − 1 | mHPRT) − 2 | Mean |
| WV-41826 | WV-41828 | 21.88 | 30.01 | 25.95 | 53.27 | 41.12 | 47.20 | 70.81 | 60.92 | 65.86 |
| WV-46568 | WV-42080 | 20.39 | 19.90 | 20.14 | 22.35 | 21.08 | 21.71 | 47.28 | 46.64 | 46.96 |
| WV-47127 | WV-42080 | 16.43 | 20.28 | 18.35 | 22.60 | 21.81 | 22.21 | 43.50 | 35.69 | 39.59 |
| WV-47129 | WV-42080 | 19.79 | 20.79 | 20.29 | 23.97 | 20.50 | 22.24 | 53.41 | 51.87 | 52.64 |
| WV-46552 | WV-42080 | 20.02 | 22.36 | 21.19 | 23.13 | 24.48 | 23.81 | 44.42 | 39.06 | 41.74 |
| WV-44452 | WV-42080 | 19.40 | 21.47 | 20.43 | 25.11 | 23.30 | 24.21 | 53.57 | 44.48 | 49.03 |
| WV-47111 | WV-42080 | 17.91 | 22.58 | 20.24 | 25.73 | 23.45 | 24.59 | 32.72 | 31.88 | 32.30 |
| WV-46571 | WV-42080 | 19.99 | 19.29 | 19.64 | 25.16 | 24.43 | 24.79 | 37.65 | 47.16 | 42.41 |
| WV-47075 | WV-42080 | 17.86 | 20.81 | 19.33 | 27.70 | 22.00 | 24.85 | 48.49 | 39.84 | 44.17 |
| WV-47085 | WV-42080 | 17.86 | 20.81 | 19.34 | 27.70 | 22.00 | 24.85 | 48.49 | 39.84 | 44.17 |
| WV-46572 | WV-42080 | 19.23 | 25.25 | 22.24 | 23.96 | 27.50 | 25.73 | 45.55 | 45.34 | 45.45 |
| WV-44453 | WV-42080 | 21.66 | 16.94 | 19.30 | 27.75 | 23.78 | 25.77 | 45.62 | 31.56 | 38.59 |
| WV-46530 | WV-42080 | 18.07 | 24.48 | 21.28 | 27.38 | 24.49 | 25.94 | 41.15 | 49.46 | 45.30 |
| WV-47121 | WV-42080 | 17.04 | 24.84 | 20.94 | 27.94 | 27.38 | 27.66 | 35.71 | 38.12 | 36.91 |
| WV-46570 | WV-42080 | 21.54 | 18.79 | 20.17 | 27.27 | 28.43 | 27.85 | 48.02 | 45.00 | 46.51 |
| WV-46527 | WV-42080 | 21.11 | 18.56 | 19.83 | 30.47 | 25.63 | 28.05 | 56.34 | 53.41 | 54.88 |
| WV-47109 | WV-42080 | 23.46 | 23.84 | 23.65 | 29.19 | 26.99 | 28.09 | 56.07 | 39.41 | 47.74 |
| WV-43775 | WV-42080 | 16.52 | 16.52 | 16.52 | 34.22 | 22.27 | 28.25 | 49.75 | 43.74 | 46.74 |
| WV-46508 | WV-42080 | 22.35 | 19.27 | 20.81 | 29.79 | 27.29 | 28.54 | 50.61 | 42.03 | 46.32 |
| WV-43774 | WV-42080 | 22.99 | 18.39 | 20.69 | 28.58 | 30.72 | 29.65 | 51.50 | 52.12 | 51.81 |
| WV-47091 | WV-42080 | 22.23 | 27.71 | 24.97 | 33.50 | 26.02 | 29.76 | 58.72 | 52.11 | 55.41 |
| WV-45148 | WV-42080 | 23.50 | 20.69 | 22.09 | 29.72 | 29.85 | 29.79 | 51.38 | 43.45 | 47.42 |
| WV-45147 | WV-42080 | 23.74 | 25.98 | 24.86 | 33.42 | 31.00 | 32.21 | 55.79 | 47.67 | 51.73 |
| WV-47124 | WV-42080 | 22.05 | 18.36 | 20.21 | 33.41 | 31.08 | 32.24 | 45.84 | 42.36 | 44.10 |
| WV-46528 | WV-42080 | 24.28 | 26.32 | 25.30 | 33.94 | 31.80 | 32.87 | 49.12 | 49.75 | 49.43 |
| WV-46532 | WV-42080 | 28.26 | 25.00 | 26.63 | 38.52 | 29.97 | 34.25 | 33.83 | 44.52 | 39.17 |
| WV-46506 | WV-42080 | 30.48 | 29.90 | 30.19 | 36.56 | 32.55 | 34.55 | 59.07 | 46.60 | 52.84 |
| WV-46553 | WV-42080 | 22.33 | 19.01 | 20.67 | 31.14 | 38.00 | 34.57 | 42.26 | 37.59 | 39.92 |
| WV-46507 | WV-42080 | 26.71 | 27.45 | 27.08 | 41.33 | 33.31 | 37.32 | 35.55 | 36.45 | 36.00 |
| WV-47106 | WV-42080 | 26.28 | 35.11 | 30.70 | 44.16 | 32.36 | 38.26 | 54.65 | 54.65 | 54.65 |
| WV-47136 | WV-42080 | 41.01 | 49.45 | 45.23 | 46.75 | 39.12 | 42.94 | 63.25 | 56.80 | 60.02 |
| WV-46509 | WV-42080 | 31.87 | 24.71 | 28.29 | 41.03 | 46.22 | 43.62 | 52.57 | 45.08 | 48.83 |
| WV-47070 | WV-42080 | 33.39 | 37.71 | 35.55 | 48.50 | 39.85 | 44.17 | 37.68 | 51.72 | 44.70 |
| WV-47118 | WV-42080 | 31.46 | 35.84 | 33.65 | 51.08 | 41.23 | 46.15 | 71.14 | 61.40 | 66.27 |
| WV-47077 | WV-42080 | 32.59 | 27.67 | 30.13 | 46.73 | 46.89 | 46.81 | 55.18 | 41.64 | 48.41 |
| WV-47093 | WV-42080 | 64.91 | 56.31 | 60.61 | 73.06 | 82.94 | 78.00 | 100.76 | 92.06 | 96.41 |

Table 5 shows % mouse TTR mRNA remaining (at 300, 100 and 30 pM siRNA treatment) relative to mouse HPRT control. N=2. N.D.: Not determined.

TABLE 5

| | | 300 pM | | | 100 pM | | | 30 pM | | |
|---|---|---|---|---|---|---|---|---|---|---|
| | | % remainin | % remainin | | % remainin | % remainin | | % remainin | % remainin | |
| | | mRNA | mRNA | | mRNA | mRNA | | mRNA | mRNA | |
| | | (mTTR/ | (mTTR/ | | (mTTR/ | (mTTR/ | | (mTTR/ | (mTTR/ | |
| Guide | Passenger | mHPRT) − 1 | mHPRT) − 2 | Mean | mHPRT) − 1 | mHPRT) − 2 | Mean | mHPRT) − 1 | mHPRT) − 2 | Mean |
| WV-41826 | WV-41828 | 16.69 | 10.42 | 13.55 | 52.05 | 48.70 | 50.37 | 77.34 | 68.47 | 72.91 |
| WV-43775 | WV-42080 | 8.07 | 8.24 | 8.16 | 36.74 | 31.07 | 33.91 | 57.26 | 66.02 | 61.64 |
| WV-46380 | WV-42080 | 19.93 | 17.32 | 18.63 | 61.23 | 54.04 | 57.63 | 93.34 | 76.71 | 85.03 |
| WV-46381 | WV-42080 | 11.29 | 15.54 | 13.41 | 35.82 | 57.47 | 46.64 | 59.95 | 81.96 | 70.95 |
| WV-46382 | WV-42080 | 13.65 | 9.19 | 11.42 | 38.13 | 45.39 | 41.76 | 89.90 | 75.80 | 82.85 |
| WV-46383 | WV-42080 | 9.69 | 10.15 | 9.92 | 39.37 | 33.25 | 36.31 | 77.05 | 72.62 | 74.83 |
| WV-46384 | WV-42080 | 5.29 | 6.91 | 6.10 | 21.37 | 24.41 | 22.89 | 59.22 | 52.96 | 56.09 |
| WV-46385 | WV-42080 | 5.11 | 4.88 | 4.99 | 20.35 | 19.95 | 20.15 | N.D. | 52.31 | 52.31 |
| WV-46386 | WV-42080 | 5.16 | 7.62 | 6.39 | 26.19 | 27.11 | 26.65 | 53.89 | 55.14 | 54.52 |
| WV-42079 | WV-42080 | 8.40 | 5.56 | 6.98 | 33.20 | 25.63 | 29.42 | 57.44 | 54.49 | 55.96 |
| WV-44434 | WV-42080 | 32.34 | 24.28 | 28.31 | 77.85 | 72.35 | 75.10 | 104.07 | 82.88 | 93.48 |
| WV-44435 | WV-42080 | 16.60 | 16.21 | 16.41 | 43.31 | 51.61 | 47.46 | N.D. | 80.54 | 80.54 |
| WV-44436 | WV-42080 | 24.72 | 16.52 | 20.62 | 56.71 | 63.51 | 60.11 | 95.72 | 86.30 | 91.01 |
| WV-44437 | WV-42080 | 17.66 | 13.07 | 15.36 | 62.38 | 56.06 | 59.22 | 92.79 | 78.34 | 85.57 |
| WV-44438 | WV-42080 | 9.41 | 5.35 | 7.38 | 33.11 | 26.93 | 30.02 | 63.34 | 57.09 | 60.21 |
| WV-44439 | WV-42080 | 8.63 | 7.69 | 8.16 | 31.16 | 27.20 | 29.18 | 60.63 | 57.41 | 59.02 |
| WV-44440 | WV-42080 | 7.33 | 10.28 | 8.81 | 34.09 | 30.58 | 32.34 | 61.32 | 62.03 | 61.68 |
| WV-44441 | WV-42080 | 10.02 | 8.51 | 9.26 | 40.40 | 38.35 | 39.38 | 66.77 | 58.26 | 62.52 |
| WV-43774 | WV-42080 | 13.20 | 7.65 | 10.42 | 40.48 | 32.53 | 36.50 | 72.94 | 60.66 | 66.80 |
| WV-46387 | WV-42080 | 13.06 | 12.26 | 12.66 | 42.91 | 47.93 | 45.42 | 92.40 | 72.40 | 82.40 |
| WV-46388 | WV-42080 | 14.83 | 10.10 | 12.47 | 45.47 | 49.69 | 47.58 | 94.82 | 77.07 | 85.94 |
| WV-46389 | WV-42080 | 8.95 | 10.29 | 9.62 | 38.54 | 40.12 | 39.33 | 86.28 | 73.34 | 79.81 |
| WV-46390 | WV-42080 | 13.48 | 11.21 | 12.34 | 41.09 | 40.30 | 40.70 | 79.66 | 64.59 | 72.12 |
| WV-46391 | WV-42080 | 7.22 | 5.94 | 6.58 | 27.09 | 23.26 | 25.17 | 53.51 | 49.24 | 51.37 |
| WV-46392 | WV-42080 | 6.55 | 8.13 | 7.34 | 28.69 | 26.54 | 27.62 | 60.65 | 56.16 | 58.41 |
| WV-46393 | WV-42080 | 8.69 | 6.61 | 7.65 | 28.89 | 27.06 | 27.97 | 61.61 | 58.77 | 60.19 |
| WV-42078 | WV-42080 | 10.30 | 8.40 | 9.35 | 36.94 | 36.31 | 36.63 | 61.61 | 65.03 | 63.32 |
| WV-46394 | WV-42080 | 12.97 | 14.39 | 13.68 | 44.61 | 54.23 | 49.42 | 90.73 | 69.75 | 80.24 |
| WV-46395 | WV-42080 | 14.35 | 11.49 | 12.92 | 49.51 | 55.10 | 52.30 | 92.53 | 73.54 | 83.04 |
| WV-46396 | WV-42080 | 12.42 | 11.56 | 11.99 | 48.06 | 50.63 | 49.34 | 85.46 | 81.32 | 83.39 |
| WV-46397 | WV-42080 | 14.32 | 12.73 | 13.53 | 44.05 | 53.42 | 48.73 | 82.39 | 72.37 | 77.38 |
| WV-46398 | WV-42080 | 7.11 | 7.23 | 7.17 | 25.40 | 24.87 | 25.13 | 54.44 | 51.35 | 52.89 |
| WV-46399 | WV-42080 | 8.00 | 6.93 | 7.46 | 28.70 | 33.00 | 30.85 | 56.36 | 49.59 | 52.97 |
| WV-46400 | WV-42080 | 9.02 | 7.47 | 8.24 | 42.97 | 32.72 | 37.85 | 64.84 | 59.30 | 62.07 |

Table 6 shows % mouse TTR mRNA remaining (at 2000 and 200 pM siRNA treatment) relative to mouse HPRT control. N=2. N.D.: Not determined.

TABLE 6

| | | 2000 pM | | | 200 pM | | |
|---|---|---|---|---|---|---|---|
| | | % remaining | % remaining | | % remaining | % remaining | |
| | | mRNA (mTTR/ | mRNA (mTTR/ | | mRNA (mTTR/ | mRNA (mTTR/ | |
| Guide | Passenger | mHPRT) − 1 | mHPRT) − 2 | Mean | mHPRT) − 1 | mHPRT) − 2 | Mean |
| WV-41826 | WV-41828 | 1.51 | 1.21 | 1.36 | 3.20 | 1.48 | 2.34 |
| WV-43775 | WV-42080 | 0.96 | 0.93 | 0.94 | 4.65 | 4.40 | 4.53 |
| WV-43774 | WV-42080 | 1.41 | 1.27 | 1.34 | 6.31 | 3.67 | 4.99 |
| WV-42079 | WV-42080 | 2.12 | 1.53 | 1.82 | 13.21 | 5.34 | 9.28 |
| WV-42078 | WV-42080 | 2.64 | 2.32 | 2.48 | 8.96 | 5.76 | 7.36 |
| WV-46991 | WV-42080 | 1.53 | 1.89 | 1.71 | 4.66 | 2.63 | 3.65 |
| WV-46992 | WV-42080 | 1.19 | 1.41 | 1.30 | 3.62 | 4.81 | 4.22 |
| WV-43988 | WV-42080 | 1.54 | 1.25 | 1.40 | 5.50 | 6.69 | 6.09 |
| WV-46993 | WV-42080 | 3.17 | 2.46 | 2.82 | 22.60 | 15.90 | 19.25 |
| WV-46997 | WV-42080 | 1.19 | 1.29 | 1.24 | 10.46 | 8.38 | 9.42 |
| WV-46998 | WV-42080 | 19.09 | 21.94 | 20.52 | 76.62 | 46.04 | 61.33 |
| WV-46999 | WV-42080 | 2.41 | 2.03 | 2.22 | 13.74 | 8.47 | 11.11 |
| WV-47000 | WV-42080 | 1.84 | 1.57 | 1.71 | 7.29 | 5.86 | 6.57 |
| WV-47001 | WV-42080 | 1.75 | 2.39 | 2.07 | 8.62 | 6.54 | 7.58 |
| WV-47002 | WV-42080 | 4.26 | 3.29 | 3.77 | 17.88 | 16.66 | 17.27 |
| WV-47006 | WV-42080 | 1.40 | 1.19 | 1.29 | 6.90 | 6.44 | 6.67 |
| WV-47007 | WV-42080 | 1.22 | 1.05 | 1.14 | 6.54 | 3.75 | 5.14 |
| WV-47008 | WV-42080 | 2.05 | 2.39 | 2.22 | 5.00 | 3.17 | 4.09 |
| WV-41825 | WV-42080 | 1.81 | 2.23 | 2.02 | 12.86 | 6.35 | 9.60 |
| WV-43771 | WV-42080 | 1.59 | 1.04 | 1.31 | 5.28 | 2.50 | 3.89 |
| WV-43773 | WV-42080 | 2.13 | 1.35 | 1.74 | 8.97 | 6.50 | 7.74 |
| WV-43770 | WV-42080 | 1.75 | 2.26 | 2.00 | 8.50 | 6.29 | 7.40 |
| WV-43772 | WV-42080 | 1.66 | 1.89 | 1.78 | 7.83 | 6.97 | 7.40 |
| WV-47009 | WV-42080 | 1.37 | 1.20 | 1.28 | 8.13 | 5.31 | 6.72 |
| WV-47010 | WV-42080 | 1.13 | 0.91 | 1.02 | 8.18 | 3.94 | 6.06 |
| WV-43996 | WV-42080 | 1.43 | 1.32 | 1.38 | 5.77 | 4.18 | 4.97 |
| WV-47011 | WV-42080 | 2.72 | 3.18 | 2.95 | 24.66 | 16.16 | 20.41 |
| WV-47015 | WV-42080 | 1.53 | 1.14 | 1.33 | 7.02 | 5.05 | 6.03 |
| WV-47016 | WV-42080 | 14.69 | 16.02 | 15.35 | 53.13 | 43.42 | 48.28 |
| WV-47017 | WV-42080 | 1.55 | 2.18 | 1.87 | 9.02 | 7.61 | 8.32 |
| WV-47018 | WV-42080 | 1.12 | 0.72 | 0.92 | 5.28 | 4.90 | 5.09 |
| WV-47019 | WV-42080 | 1.48 | 1.10 | 1.29 | 6.69 | 5.34 | 6.01 |
| WV-47020 | WV-42080 | 3.26 | 2.84 | 3.05 | 23.45 | 13.64 | 18.55 |
| WV-47024 | WV-42080 | 1.26 | 1.19 | 1.23 | 8.28 | 4.84 | 6.56 |
| WV-47025 | WV-42080 | 1.35 | 1.46 | 1.40 | 14.38 | 10.63 | 12.51 |
| WV-47026 | WV-42080 | 2.03 | 1.65 | 1.84 | 6.23 | 6.21 | 6.22 |

Table 7 shows % IC50 of knocking down mouse TTR mRNA in mouse primary hepatocyte

TABLE 7

| Guide | Passenger | IC50 (pM) | 95% CI |
|---|---|---|---|
| WV-41826 | WV-41828 | 82.55 | 54.44 to 127.4 |
| WV-43774 | WV-42080 | 47.97 | 36.22 to 63.82 |
| WV-42078 | WV-42080 | 110.6 | 80.64 to 153.8 |
| WV-45148 | WV-42080 | 39.22 | 26.69 to 58.11 |
| WV-47085 | WV-42080 | 32.47 | 24.07 to 43.86 |
| WV-45147 | WV-42080 | 52.43 | 34.70 to 80.0 |
| WV-47091 | WV-42080 | 30.84 | 22.07 to 43.32 |
| WV-47144 | WV-42080 | 22.45 | 16.98 to 29.73 |
| WV-41826 | WV-41828 | 50.57 | 27.54 to 93.72 |
| WV-43775 | WV-42080 | 26.49 | 19.04 to 36.93 |
| WV-42079 | WV-42080 | 35.42 | 25.06 to 50.22 |
| WV-44453 | WV-42080 | 18.57 | 12.61 to 27.39 |
| WV-47121 | WV-42080 | 16.67 | 12.57 to 22.12 |
| WV-44452 | WV-42080 | 22.77 | 15.09 to 34.42 |
| WV-47127 | WV-42080 | 13.77 | 8.85 to 21.34 |
| WV-47145 | WV-42080 | 20.15 | 13.41 to 30.47 |

### Example 3. Provided Oligonucleotides and Compositions are Active In Vivo

In vivo determination of mouse TTR siRNA activity: All animal procedures were performed under IACUC guidelines at Alpha Preclinical (North Grafton, MA). To evaluate the durability of provided oligonucleotides and compositions, male 8-10 weeks of age C57BL/6 mice were dose at 1.5 mg/kg at desired oligonucleotide concentration on Day 1 by subcutaneous administration. On Day 1 (pre-dose) and then weekly, whole blood was collected by tail snip into serum separator tubes, and processed serum samples were kept at −70° C. Mouse TTR protein concentration in the serum was assessed using the Mouse Prealbumin ELISA kit (Crystal Chem) and following manufacturer's instructions.

Table 8 shows % mouse TTR protein remaining relative to PBS control. N=5. N.D.: Not determined.

TABLE 8

| | PBS | | | | | |
|---|---|---|---|---|---|---|
| Day | animal1 | animal2 | animal3 | animal4 | animal5 | Mean |
| 1 | 90 | 111 | 100 | 103 | 95 | 100 |
| 8 | 91 | 112 | 102 | 113 | 82 | 100 |
| 15 | 102 | 99 | 112 | 100 | 86 | 100 |
| 22 | 101 | 101 | 98 | 103 | 96 | 100 |
| 29 | 108 | 101 | 110 | 94 | 87 | 100 |
| 36 | 94 | 102 | 106 | 96 | 103 | 100 |
| 43 | 99 | 85 | 125 | 99 | 91 | 100 |

| | WV-41826/WV-41828, 1.5 mg/kg | | | | | |
|---|---|---|---|---|---|---|
| Day | animal6 | animal7 | animal 8 | animal9 | animal10 | Mean |
| 1 | 111 | 86 | 77 | 117 | 144 | 107 |
| 8 | 1 | 2 | 4 | 4 | 4 | 3 |
| 15 | 2 | 4 | 4 | 8 | 6 | 5 |
| 22 | 6 | 9 | 11 | 5 | 8 | 8 |
| 29 | 19 | 20 | 22 | 19 | 13 | 18 |
| 36 | 24 | 30 | 41 | 28 | 26 | 30 |
| 43 | 55 | 45 | 47 | 42 | 45 | 47 |

| | WV-43775/WV-42080, 1.5 mg/kg | | | | | |
|---|---|---|---|---|---|---|
| Day | animal11 | animal12 | animal 13 | animal14 | animal15 | Mean |
| 1 | 113 | 104 | 122 | 86 | 90 | 103 |
| 8 | 4 | N.D. | N.D. | N.D. | N.D. | 4 |
| 15 | 8 | 1 | 2 | 1 | 2 | 3 |
| 22 | 12 | 5 | 8 | 6 | 6 | 8 |
| 29 | 15 | 13 | 19 | 17 | 15 | 16 |
| 36 | 38 | 23 | 38 | 31 | 31 | 32 |
| 43 | 61 | 44 | 64 | 65 | 55 | 58 |
| | WV-42079/WV-42080, 1.5 mg/kg | | | | | |
| Day | animal16 | animal17 | animal18 | animal19 | animal20 | Mean |
| 1 | 83 | 123 | 127 | 118 | 74 | 105 |
| 8 | N.D. | 5 | 1 | 3 | 2 | 3 |
| 15 | 2 | 7 | 2 | 6 | 4 | 4 |
| 22 | 11 | 9 | 8 | 8 | 8 | 9 |
| 29 | 24 | 21 | 14 | 16 | 20 | 19 |
| 36 | 44 | 37 | 32 | 25 | 40 | 35 |
| 43 | 59 | 51 | 64 | 47 | 54 | 55 |
| | WV-43771/WV-42080, 1.5 mg/kg | | | | | |
| Day | animal21 | animal22 | animal23 | animal24 | animal25 | Mean |
| 1 | 107 | 67 | 104 | 73 | 93 | 89 |
| 8 | 3 | N.D. | 2 | N.D. | N.D. | 3 |
| 15 | 6 | 3 | 4 | 2 | 1 | 3 |
| 22 | 1 | 10 | 7 | 5 | 6 | 6 |
| 29 | 32 | 21 | 19 | 18 | 16 | 21 |
| 36 | 49 | 55 | 23 | 84 | 32 | 49 |
| 43 | 74 | 64 | 46 | 61 | 55 | 60 |
| | WV-43773/WV-42080, 1.5 mg/kg | | | | | |
| Day | animal26 | animal27 | animal28 | animal29 | animal30 | Mean |
| 1 | 126 | 108 | 107 | 98 | 94 | 107 |
| 8 | 5 | 5 | 6 | 4 | 6 | 5 |
| 15 | 7 | 5 | 7 | 8 | 4 | 6 |
| 22 | 15 | 13 | 13 | 10 | 9 | 12 |
| 29 | 34 | 33 | 27 | 19 | 22 | 27 |
| 36 | 61 | 59 | 46 | 31 | 38 | 47 |
| 43 | 82 | 82 | 68 | 60 | 64 | 71 |
| | WV-43988/WV-42080, 1.5 mg/kg | | | | | |
| Day | animal31 | animal32 | animal33 | animal34 | animal35 | Mean |
| 1 | 104 | 101 | 105 | 126 | 120 | 111 |
| 8 | N.D. | 1 | 3 | 1 | 5 | 2 |
| 15 | 1 | 1 | 2 | 1 | 1 | 1 |
| 22 | 4 | 4 | 5 | 4 | 7 | 5 |
| 29 | 8 | 10 | 10 | 14 | 11 | 11 |
| 36 | 18 | 17 | 21 | 23 | 18 | 19 |
| 43 | 34 | 39 | 36 | 39 | 34 | 36 |
| | WV-43989/WV-42080, 1.5 mg/kg | | | | | |
| Day | animal36 | animal37 | animal38 | animal39 | animal40 | Mean |
| 1 | 64 | 118 | 89 | 108 | 101 | 96 |
| 8 | 4 | 4 | 1 | 12 | 3 | 5 |
| 15 | 5 | 4 | 1 | 11 | 6 | 6 |
| 22 | 10 | 8 | 7 | 24 | 15 | 13 |
| 29 | 25 | 19 | 19 | 44 | 32 | 28 |
| 36 | 45 | 38 | 42 | 69 | 75 | 54 |
| 43 | 22 | 69 | 61 | 56 | 88 | 59 |
| | WV-43994/WV-42080, 1.5 mg/kg | | | | | |
| Day | animal41 | animal42 | animal43 | animal44 | animal45 | Mean |
| 1 | 141 | 124 | 104 | 124 | 113 | 121 |
| 8 | 3 | 3 | 2 | 2 | 4 | 3 |
| 15 | 3 | 2 | 2 | 3 | 7 | 3 |
| 22 | 8 | 11 | 8 | 8 | 8 | 8 |
| 29 | 21 | 17 | 20 | 20 | 19 | 19 |
| 36 | 29 | 36 | 39 | 34 | 34 | 35 |
| 43 | 51 | 68 | 63 | 61 | 55 | 59 |
| | WV-43996/WV-42080, 1.5 mg/kg | | | | | |
| Day | animal46 | animal47 | animal48 | animal49 | animal50 | Mean |
| 1 | 106 | 71 | 89 | 110 | 118 | 99 |
| 8 | 2 | 4 | 2 | 1 | 1 | 2 |
| 15 | 2 | 2 | 2 | 2 | 1 | 2 |
| 22 | 3 | 4 | 6 | 5 | 7 | 5 |
| 29 | 8 | 10 | 11 | 15 | 13 | 11 |
| 36 | 18 | 20 | 23 | 27 | 29 | 23 |
| 43 | 31 | 36 | 37 | 38 | 40 | 36 |

| | WV-43256/WV-42080, 1.5 mg/kg | | | | |
|---|---|---|---|---|---|
| Day | animal51 | animal52 | animal53 | animal54 | Mean |
| 1 | 92 | 97 | 104 | 101 | 99 |
| 8 | 4 | 6 | 3 | 9 | 6 |
| 15 | 9 | 9 | 6 | 8 | 8 |
| 22 | 21 | 22 | 14 | 21 | 19 |
| 29 | 51 | 44 | 43 | 37 | 44 |
| 36 | 83 | 76 | 62 | 74 | 74 |
| 43 | 82 | 76 | 60 | 77 | 74 |

### Example 4. Provided Oligonucleotides and Compositions are Active In Vivo with Longer Duration

In vivo determination of mouse TTR siRNA activity: All animal procedures were performed under IACUC guidelines at Biomere (Worcester, MA). Male 8-10 weeks of age C57BL/6 mice were dose at 1 mg/kg at desired oligonucleotide concentration on Day 1 by subcutaneous administration to the interscapular area. Blood samples were collected by tail snip into serum separator tubes, and processed serum samples were kept at −70° C. Mouse TTR protein concentration in the serum was assessed using the Mouse Prealbumin ELISA kit (Novus Biologicals) and following manufacturer's instructions.

Table 9. shows % mouse TTR protein remaining relative to PBS control. N=5. N.D.: Not determined.

TABLE 9

| | PBS | | | | | |
|---|---|---|---|---|---|---|
| Day | animal1 | animal2 | animal3 | animal4 | animal5 | Mean |
| 1 | 100 | 96 | 97 | 110 | 98 | 100 |
| 8 | 97 | 106 | 100 | 103 | 93 | 100 |
| 15 | 92 | 101 | 97 | 118 | 92 | 100 |
| 22 | 99 | 101 | 100 | 111 | 90 | 100 |
| 29 | 98 | 101 | 101 | 98 | 102 | 100 |
| 36 | 100 | 95 | 84 | 121 | 102 | 100 |
| 43 | 79 | 109 | 93 | 124 | 95 | 100 |
| 50 | 97 | 102 | 97 | 107 | 97 | 100 |
| 57 | 98 | 94 | 110 | 98 | 101 | 100 |
| 64 | 116 | 86 | 102 | 112 | 84 | 100 |
| 71 | 86 | 93 | 100 | 127 | 94 | 100 |
| | WV-41826/WV-41828, 1 mg/kg | | | | | |
| Day | animal6 | animal7 | animal8 | animal9 | animal10 | Mean |
| 1 | 89 | 124 | 96 | 111 | 104 | 105 |
| 8 | 11 | 14 | 12 | 13 | 13 | 12 |
| 15 | 6 | 10 | 8 | 7 | 8 | 8 |
| 22 | 9 | 12 | 11 | 11 | 11 | 11 |
| 29 | 18 | 24 | 22 | 46 | 18 | 26 |
| 36 | 24 | 31 | 30 | 32 | 31 | 30 |
| 43 | 41 | 53 | 54 | 49 | 50 | 49 |
| 50 | 67 | 80 | 77 | 75 | 83 | 76 |
| 57 | 72 | 84 | 79 | 74 | 80 | 78 |
| 64 | 80 | 97 | 90 | 70 | 70 | 82 |
| 71 | 72 | 97 | 77 | 90 | 92 | 85 |
| | WV-42078/WV-42080, 1 mg/kg | | | | | |
| Day | animal11 | animal12 | animal13 | animal14 | animal15 | Mean |
| 1 | 88 | 101 | 111 | 113 | 122 | 107 |
| 8 | 9 | 15 | 10 | 12 | 12 | 12 |
| 15 | 2 | 10 | 4 | 5 | 6 | 5 |
| 22 | 4 | 15 | 6 | 5 | 9 | 8 |
| 29 | 7 | 25 | 9 | 11 | 13 | 13 |
| 36 | 19 | 42 | 15 | 19 | 22 | 23 |
| 43 | 36 | 40 | 24 | 38 | 33 | 34 |
| 50 | 63 | 67 | 48 | 61 | 63 | 60 |
| 57 | 72 | 64 | 71 | 82 | 73 | 72 |
| 64 | 72 | 54 | 84 | 68 | 70 | 70 |
| 71 | 95 | 77 | 65 | 75 | 89 | 80 |
| | WV-43774/WV-42080, 1 mg/kg | | | | | |
| Day | animal16 | animal17 | animal18 | animal19 | animal20 | Mean |
| 1 | 104 | 88 | 92 | 96 | 101 | 96 |
| 8 | 11 | 6 | 8 | 8 | 8 | 8 |
| 15 | 6 | 3 | 3 | 3 | 4 | 4 |
| 22 | 9 | 5 | 6 | 7 | 7 | 7 |
| 29 | 16 | 11 | 15 | 18 | 14 | 15 |
| 36 | 26 | 23 | 19 | 21 | 23 | 22 |
| 43 | 34 | 34 | 41 | 47 | 37 | 39 |
| 50 | 66 | 63 | 71 | 68 | 73 | 68 |
| 57 | 71 | 65 | 71 | 79 | 84 | 74 |
| 64 | 83 | 98 | 94 | 75 | 73 | 84 |
| 71 | 81 | 93 | 76 | 83 | 86 | 84 |
| | WV-47085/WV-42080, 1 mg/kg | | | | | |
| Day | animal21 | animal22 | animal23 | animal24 | animal25 | Mean |
| 1 | 80 | 119 | 83 | 112 | 106 | 100 |
| 8 | 19 | 7 | 17 | 11 | 9 | 13 |
| 15 | 17 | 4 | 11 | 6 | 5 | 9 |
| 22 | 29 | 8 | 17 | 11 | 9 | 15 |
| 29 | 40 | 16 | 30 | 25 | 23 | 27 |
| 36 | 50 | 27 | 36 | 40 | 36 | 38 |
| 43 | 53 | 36 | 44 | 56 | 54 | 49 |
| 50 | 83 | 63 | 52 | 94 | 86 | 76 |
| 57 | 88 | 75 | 67 | 81 | 74 | 77 |
| 64 | 85 | 46 | 36 | 81 | 106 | 71 |
| 71 | 69 | 62 | 44 | 103 | 108 | 77 |
| | WV-47091/WV-42080, 1 mg/kg | | | | | |
| Day | animal26 | animal27 | animal28 | animal29 | animal30 | Mean |
| 1 | 83 | 86 | 91 | 101 | 99 | 92 |
| 8 | 13 | 7 | 6 | 8 | 7 | 8 |
| 15 | 6 | 3 | 2 | 3 | 3 | 3 |
| 22 | 9 | 5 | 3 | 5 | 5 | 5 |
| 29 | 15 | 11 | 6 | 7 | 8 | 9 |
| 36 | 26 | 20 | 12 | 12 | 15 | 17 |
| 43 | 34 | 31 | 20 | 16 | 23 | 25 |
| 50 | 44 | 60 | 43 | 35 | 57 | 48 |
| 57 | 47 | 49 | 44 | 37 | 50 | 45 |
| 64 | 54 | 62 | 69 | 59 | 77 | 64 |
| 71 | 70 | 58 | 66 | 72 | 66 | 66 |
| | WV-47144/WV-42080, 1 mg/kg | | | | | |
| Day | animal31 | animal32 | animal33 | animal34 | animal35 | Mean |
| 1 | 113 | 112 | 106 | 120 | 105 | 111 |
| 8 | 11 | 11 | 10 | 11 | 11 | 11 |
| 15 | 5 | 5 | 4 | 4 | 5 | 5 |
| 22 | 7 | 7 | 6 | 6 | 7 | 7 |
| 29 | 11 | 12 | 11 | 20 | 11 | 13 |
| 36 | 16 | 15 | 17 | 15 | 16 | 16 |
| 43 | 24 | 21 | 21 | 21 | 23 | 22 |
| 50 | 46 | 45 | 46 | 54 | 49 | 48 |
| 57 | 58 | 48 | 61 | 55 | 44 | 53 |
| 64 | 65 | 54 | 68 | 62 | 59 | 61 |
| 71 | 61 | 74 | 91 | 93 | 55 | 75 |
| | WV-47121/WV-42080, 1 mg/kg | | | | | |
| Day | animal36 | animal37 | animal38 | animal39 | animal40 | Mean |
| 1 | 106 | 76 | 104 | 130 | 94 | 102 |
| 8 | 6 | 6 | 10 | 9 | 9 | 8 |
| 15 | 3 | 3 | 7 | 5 | 5 | 4 |
| 22 | 8 | 10 | 10 | 9 | 9 | 9 |
| 29 | 17 | 20 | 23 | 18 | 17 | 19 |
| 36 | 36 | 39 | 35 | 32 | 41 | 36 |
| 43 | 42 | 53 | 46 | 51 | 49 | 48 |
| 50 | 83 | 101 | 89 | 64 | 78 | 83 |
| 57 | 69 | 65 | 87 | 80 | 96 | 79 |
| 64 | 59 | 67 | 96 | 86 | 80 | 78 |
| 71 | 76 | 87 | 118 | 120 | 136 | 107 |
| | WV-47127/WV-42080, 1 mg/kg | | | | | |
| Day | animal41 | animal42 | animal43 | animal44 | animal45 | Mean |
| 1 | 75 | 78 | 91 | 97 | 96 | 87 |
| 8 | 7 | 7 | 7 | 12 | 7 | 8 |
| 15 | 2 | 3 | 2 | 5 | 3 | 3 |
| 22 | 4 | 6 | 5 | 8 | 5 | 6 |
| 29 | 7 | 10 | 12 | 14 | 9 | 10 |
| 36 | 13 | 27 | 19 | 20 | 15 | 19 |
| 43 | 22 | 39 | 50 | 32 | 23 | 33 |
| 50 | 52 | 66 | 54 | 51 | 54 | 56 |
| 57 | 67 | 66 | 79 | 72 | 66 | 70 |
| 64 | 129 | 90 | 79 | 73 | 73 | 89 |
| 71 | 93 | 81 | 95 | 106 | 81 | 91 |
| | WV-47145/WV-42080, 1 mg/kg | | | | | |
| Day | animal46 | animal47 | animal48 | animal49 | animal50 | Mean |
| 1 | 99 | 101 | 98 | 93 | 112 | 101 |
| 8 | 9 | 7 | 9 | 8 | 10 | 9 |
| 15 | 4 | 2 | 5 | 3 | 5 | 4 |
| 22 | 7 | 5 | 7 | 5 | 4 | 5 |
| 29 | 12 | 6 | 12 | 7 | 9 | 9 |
| 36 | 17 | 12 | 13 | 11 | 13 | 13 |
| 43 | 24 | 16 | 23 | 17 | 22 | 20 |
| 50 | 52 | 35 | 42 | 40 | 34 | 41 |
| 57 | 44 | 40 | 49 | 53 | 40 | 45 |
| 64 | 59 | 49 | 56 | 60 | 65 | 58 |
| 71 | 95 | 61 | 72 | 73 | 66 | 73 |

### Example 5. Provided Oligonucleotides and Compositions can Effectively Knockdown Mouse Transthyretin (mTTR) In Vivo with Enhanced Potency

In vivo determination of mouse TTR siRNA activity: All animal procedures were performed under IACUC guidelines at Biomere (Worcester, MA). Male 8-10 weeks of age C57BL/6 mice were dose at 0.5 mg/kg at desired oligonucleotide concentration on Day 1 by subcutaneous administration to the interscapular area. Blood samples were collected by tail snip into serum separator tubes, and processed serum samples were kept at −70° C. Mouse TTR protein concentration in the serum was assessed using the Mouse Prealbumin ELISA kit (Novus Biologicals) and following manufacturer's instructions.

Table 10 shows % mouse TTR protein remaining relative to PBS control. N=5. N.D.: Not determined.

TABLE 10

| | PBS | | | | | |
|---|---|---|---|---|---|---|
| Day | animal1 | animal2 | animal3 | animal4 | animal5 | Mean |
| 1 | 85 | 96 | 93 | 104 | 123 | 100 |
| 8 | 92 | 108 | 98 | 99 | 102 | 100 |
| 15 | 110 | 104 | 90 | 99 | 97 | 100 |
| 22 | 104 | 118 | 84 | 82 | 113 | 100 |
| 29 | 108 | 107 | 91 | 95 | 99 | 100 |
| 36 | 106 | 98 | 99 | 112 | 85 | 100 |
| 43 | 124 | 119 | 85 | 86 | 86 | 100 |
| | WV-41826/WV-41828, 0.5 mg/kg | | | | | |
| Day | animal6 | animal7 | animal 8 | animal9 | animal10 | Mean |
| 1 | 83 | 93 | 121 | 102 | 80 | 96 |
| 8 | 48 | 17 | 25 | 20 | 20 | 26 |
| 15 | 51 | 19 | 27 | 19 | 23 | 28 |
| 22 | 60 | 24 | 35 | 31 | 30 | 36 |
| 29 | 83 | 40 | 47 | 43 | 51 | 53 |
| 36 | 74 | 54 | 61 | 71 | 72 | 67 |
| 43 | 76 | 82 | 68 | 78 | 88 | 78 |
| | WV-43774/WV-42080, 0.5 mg/kg | | | | | |
| Day | animal11 | animal12 | animal13 | animal14 | animal15 | Mean |
| 1 | 76 | 92 | 103 | 83 | 108 | 92 |
| 8 | 11 | 16 | 16 | 33 | 50 | 25 |
| 15 | 41 | 18 | 18 | 13 | 54 | 29 |
| 22 | 17 | 26 | 25 | 48 | 60 | 35 |
| 29 | 28 | 49 | 38 | 81 | 81 | 55 |
| 36 | 58 | 71 | 56 | 100 | 97 | 76 |
| 43 | 98 | 98 | 83 | 96 | 91 | 93 |
| | WV-43775/WV-42080, 0.5 mg/kg | | | | | |
| Day | animal16 | animal17 | animal18 | animal19 | animal20 | Mean |
| 1 | 82 | 86 | 86 | 87 | 94 | 87 |
| 8 | 5 | 117 | 10 | 7 | 92 | 46 |
| 15 | 8 | 76 | 11 | 8 | 86 | 38 |
| 22 | 14 | 86 | 15 | 16 | 69 | 40 |
| 29 | 42 | 100 | 47 | 34 | 97 | 64 |
| 36 | 57 | 97 | 62 | 57 | 89 | 72 |
| 43 | 80 | 112 | 80 | 77 | 94 | 89 |
| | WV-48528/WV-42080, 0.5 mg/kg | | | | | |
| Day | animal21 | animal22 | animal23 | animal24 | animal25 | Mean |
| 1 | 102 | 109 | 97 | 99 | 99 | 101 |
| 8 | 8 | 8 | 9 | 5 | 6 | 7 |
| 15 | 9 | 7 | 9 | 6 | 5 | 7 |
| 22 | 17 | 12 | 12 | 9 | 14 | 13 |
| 29 | 26 | 31 | 31 | 20 | 28 | 27 |
| 36 | 43 | 49 | 47 | 34 | 51 | 45 |
| 43 | 79 | 92 | 89 | 62 | 78 | 80 |
| | WV-48530/WV-42080, 0.5 mg/kg | | | | | |
| Day | animal26 | animal27 | animal28 | animal29 | animal30 | Mean |
| 1 | 76 | 119 | 102 | 69 | 97 | 93 |
| 8 | 78 | 40 | 11 | 12 | 11 | 30 |
| 15 | 85 | 42 | 13 | 13 | 10 | 33 |
| 22 | 80 | 50 | 20 | 26 | 15 | 38 |
| 29 | 119 | 68 | 36 | 35 | 29 | 57 |
| 36 | 110 | 73 | 55 | 48 | 41 | 65 |
| 43 | 106 | 96 | 83 | 72 | 80 | 87 |
| | WV-48531/WV-42080, 0.5 mg/kg | | | | | |
| Day | animal31 | animal32 | animal33 | animal34 | animal35 | Mean |
| 1 | 130 | 116 | 100 | 94 | 115 | 111 |
| 8 | 71 | 15 | 49 | 10 | 15 | 32 |
| 15 | 65 | 15 | 33 | 10 | 12 | 27 |
| 22 | 18 | 22 | 63 | 20 | 78 | 40 |
| 29 | 111 | 49 | 55 | 29 | 35 | 56 |
| 36 | 108 | 62 | 98 | 57 | 48 | 74 |
| 43 | 115 | 79 | 110 | 83 | 83 | 94 |
| | WV-47145/WV-42080, 0.5 mg/kg | | | | | |
| Day | animal36 | animal37 | animal38 | animal39 | animal40 | Mean |
| 1 | 83 | 77 | 102 | 81 | 104 | 89 |
| 8 | 64 | 3 | 6 | 3 | 11 | 18 |
| 15 | 77 | 6 | 6 | 4 | 9 | 20 |
| 22 | 91 | 15 | 12 | 10 | 15 | 28 |
| 29 | 91 | 23 | 22 | 22 | 30 | 38 |
| 36 | 36 | 86 | 31 | 44 | 44 | 48 |
| 43 | 108 | 71 | 66 | 76 | 68 | 78 |

### Example 6. Provided Oligonucleotides and Compositions are Active In Vivo

In vivo determination of mouse TTR siRNA activity: All animal procedures were performed under IACUC guidelines. To evaluate the potency and liver exposure of provided oligonucleotides and compositions, male 8-10 weeks of age C57BL/6 mice were dose at 0.5 mg/kg at desired oligonucleotide concentration on Day 1 by subcutaneous administration. Animals were euthanized on Day 8 by CO₂ asphyxiation followed by thoracotomy and terminal blood collection. After cardiac perfusion with PBS, liver samples were harvested and flash-frozen in dry ice. Processed serum samples were kept at −70° C. Mouse TTR protein concentration in the serum was assessed using the Mouse Prealbumin ELISA kit (Novus Biologicals) and following manufacturer's instructions. Liver total RNA was extracted using SV96 Total RNA Isolation kit (Promega), after tissue lysis with TRIzol and bromochloropropane. cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). For mouse TTR mRNA, the following qPCR assay were utilized: IDT Taqman qPCR assay ID Mm.PT.58.11922308. Oligonucleotide accumulation in liver was determined by hybrid ELISA.

Ago2 immunoprecipitation assay: Tissues (1 mpk dosed) were lysed in lysis buffer 50 mM Tris-HCl at pH 7.5, 200 mM NaCl, 0.5% Triton X-100, 2 mM EDTA, 1 mg/mL heparin) with protease inhibitor (Sigma-Aldrich). Lysate concentration was measured with a protein BCA kit (Pierce BCA protein assay kit or Bradford protein assay kit). Anti-Ago2 antibody was purchased from Wako Chemicals. Control mouse IgG was from eBioscience. Dynabeads (Invitrogen) were used to precipitate antibodies. Ago2-associated siRNA and endogenous miR122 were measured by Stem-Loop RT followed by TaqMan PCR analysis using Taqman miRNA and siRNA assay kit (Thermo fisher) based on manufacturer's methods.

Table 11 shows % mouse TTR protein remaining relative to PBS control. N=5. N.D.: Not determined.

TABLE 11

| | | WV-41826/WV- | | WV-43774/WV- | | WV-43775/WV- | |
|---|---|---|---|---|---|---|---|
| PBS | | 41828 | | 42080 | | 42080 | |
| | % remaining | | % remaining | | % remaining | | % remaining |
| animal | of mTTR | animal | of mTTR | animal | of mTTR | animal | of mTTR |
| No. | protein | No. | protein | No. | protein | No. | protein |
| 1 | 102 | 6 | 11 | 11 | 14 | 16 | 3 |
| 2 | 107 | 7 | 10 | 12 | 9 | 17 | 4 |
| 3 | 105 | 8 | 17 | 13 | 6 | 18 | 2 |
| 4 | 92 | 9 | 11 | 14 | 6 | 19 | 6 |
| 5 | 94 | 10 | 15 | 15 | 9 | 20 | 8 |
| Mean | 100 | Mean | 13 | Mean | 9 | Mean | 4 |
| WV-47091/WV- | | WV-47144/WV- | | WV-47127/WV- | | WV-47145/WV- | |
| 42080 | | 42080 | | 42080 | | 42080 | |
| | % remaining | | % remaining | | % remaining | | % remaining |
| animal | of mTTR | animal | of mTTR | animal | of mTTR | animal | of mTTR |
| No. | protein | No. | protein | No. | protein | No. | protein |
| 21 | 8 | 26 | 8 | 31 | 9 | 36 | 6 |
| 22 | 9 | 27 | 7 | 32 | 7 | 37 | 4 |
| 23 | 9 | 28 | 6 | 33 | 9 | 38 | 11 |
| 24 | 6 | 29 | 7 | 34 | 6 | 39 | 6 |
| 25 | 9 | 30 | 5 | 35 | 13 | 40 | 5 |
| Mean | 8 | Mean | 6 | Mean | 9 | Mean | 6 |
| WV-48528/WV- | | WV-48530/WV- | | WV-48531/WV- | | | |
| 42080 | | 42080 | | 42080 | | | |
| | % remaining | | % remaining | | % remaining | | |
| animal | of mTTR | animal | of mTTR | animal | of mTTR | | |
| No. | protein | No. | protein | No. | protein | | |
| 41 | 6 | 46 | 11 | 51 | 11 | | |
| 42 | 10 | 47 | 15 | 52 | 11 | | |
| 43 | 6 | 48 | 23 | 53 | 12 | | |
| 44 | 7 | 49 | 11 | 54 | 10 | | |
| 45 | 9 | 50 | 19 | 55 | 4 | | |
| Mean | 8 | Mean | 16 | Mean | 10 | | |

Table 12. shows the accumulation of antisense strand in liver tissue. N=5. N.D.: Not determined.

TABLE 12

| | | WV-41826/WV- | | WV-43774/WV- | | WV-43775/WV- | |
|---|---|---|---|---|---|---|---|
| PBS | | 41828 | | 42080 | | 42080 | |
| | antisense | | antisense | | antisense | | antisense |
| | strand | | strand | | strand | | strand |
| animal | (μg/g of | animal | (μg/g of | animal | (μg/g of | animal | (μg/g of |
| No. | tissue) | No. | tissue) | No. | tissue) | No. | tissue) |
| 1 | 0.000 | 6 | 0.062 | 11 | 0.086 | 16 | 0.040 |
| 2 | 0.000 | 7 | 0.089 | 12 | 0.072 | 17 | 0.039 |
| 3 | 0.000 | 8 | 0.092 | 13 | 0.073 | 18 | 0.062 |
| 4 | 0.000 | 9 | 0.119 | 14 | 0.121 | 19 | 0.074 |
| 5 | 0.000 | 10 | 0.101 | 15 | 0.134 | 20 | 0.082 |
| Mean | 0.000 | Mean | 0.093 | Mean | 0.097 | Mean | 0.059 |
| WV-47091/WV- | | WV-47144/WV- | | WV-47127/WV- | | WV-47145/WV- | |
| 42080 | | 42080 | | 42080 | | 42080 | |
| | antisense | | antisense | | antisense | | antisense |
| | strand | | strand | | strand | | strand |
| animal | (μg/g of | animal | (μg/g of | animal | (μg/g of | animal | (μg/g of |
| No. | tissue) | No. | tissue) | No. | tissue) | No. | tissue) |
| 21 | 0.771 | 26 | 1.144 | 31 | 0.955 | 36 | 0.792 |
| 22 | 0.599 | 27 | 1.058 | 32 | 1.599 | 37 | 0.871 |
| 23 | 0.881 | 28 | 0.938 | 33 | 1.128 | 38 | 0.846 |
| 24 | 0.624 | 29 | 0.679 | 34 | 1.345 | 39 | 0.856 |
| 25 | 0.579 | 30 | 0.607 | 35 | 1.264 | 40 | 0.814 |
| Mean | 0.691 | Mean | 0.885 | Mean | 1.258 | Mean | 0.836 |
| WV-48528/WV- | | WV-48530/WV- | | WV-48531/WV- | | | |
| 42080 | | 42080 | | 42080 | | | |
| | antisense | | antisense | | antisense | | |
| | strand | | strand | | strand | | |
| animal | (μg/g of | animal | (μg/g of | animal | (μg/g of | | |
| No. | tissue) | No. | tissue) | No. | tissue) | | |
| 41 | 0.555 | 46 | 0.334 | 51 | 0.054 | | |
| 42 | 0.834 | 47 | 0.388 | 52 | 0.023 | | |
| 43 | 0.976 | 48 | 0.041 | 53 | 0.082 | | |
| 44 | 0.995 | 49 | 0.283 | 54 | 0.154 | | |
| 45 | 1.235 | 50 | 0.545 | 55 | 0.071 | | |
| Mean | 0.919 | Mean | 0.318 | Mean | 0.077 | | |

### Example 7. Provided Oligonucleotides and Compositions are Active In Vivo

In vivo determination of mouse TTR siRNA activity: All animal procedures were performed under IACUC guidelines. To evaluate the potency and liver exposure of provided oligonucleotides and compositions, male 8-10 weeks of age C₅₇BL/6 mice were dose at 0.6, 2 or 6 mg/kg at desired oligonucleotide concentration on Day 1 by subcutaneous administration. Animals were euthanized on Day 8 by C₀₂ asphyxiation followed by thoracotomy and terminal blood collection. After cardiac perfusion with PBS, liver samples were harvested and flash-frozen in dry ice. Liver total RNA was extracted using SV96 Total RNA Isolation kit (Promega), after tissue lysis with TRIzol and bromochloropropane. cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). For mouse TTR mRNA, the following qPCR assay were utilized: IDT Taqman qPCR assay ID Mm.PT.58.11922308. Oligonucleotide accumulation in liver was determined by hybrid ELISA.

To evaluate the durability of provided oligonucleotides and compositions, male 8-10 weeks of age C57BL/6 mice were dose at 2 or 6 mg/kg at desired oligonucleotide concentration on Day 1 by subcutaneous administration. On Day 1 (pre-dose) and then weekly, whole blood was collected via submandibular bleeding into serum separator tubes, and processed serum samples were kept at −70° C. Mouse TTR protein concentration in the serum was assessed using the Mouse Prealbumin ELISA kit (Crystal Chem) and following manufacturer's instructions.

Ago2 immunoprecipitation assay: Tissues (1 mpk dosed) were lysed in lysis buffer 50 mM Tris-HCl at pH 7.5, 200 mM NaCl, 0.5% Triton X-100, 2 mM EDTA, 1 mg/mL heparin) with protease inhibitor (Sigma-Aldrich). Lysate concentration was measured with a protein BCA kit (Pierce BCA protein assay kit or Bradford protein assay kit). Anti-Ago2 antibody was purchased from Wako Chemicals. Control mouse IgG was from eBioscience. Dynabeads (Invitrogen) were used to precipitate antibodies. Ago2-associated siRNA and endogenous miR122 were measured by Stem-Loop RT followed by TaqMan PCR analysis using Taqman miRNA and siRNA assay kit (Thermo fisher) based on manufacturer's methods.

Table 13 shows % mouse TTR mRNA remaining relative to PBS control. N=5. N.D.: Not determined.

TABLE 13

| | | WV-20167/WV-36860 | | | | | |
|---|---|---|---|---|---|---|---|
| | PBS | 0.6 mg/kg | | 2 mg/kg | | 6 mg/kg | |
| | % remaining | | % remaining | | % remaining | | % remaining |
| animal | of mTTR | animal | of mTTR | animal | of mTTR | animal | of mTTR |
| No. | mRNA | No. | mRNA | No. | mRNA | No. | mRNA |
| 1 | 126.89 | 6 | 81.73 | 11 | 38.85 | 16 | 35.76 |
| 2 | 86.61 | 7 | 70.20 | 12 | 42.98 | 17 | 17.77 |
| 3 | 95.66 | 8 | 75.48 | 13 | 40.83 | 18 | 20.10 |
| 4 | 93.00 | 9 | 65.90 | 14 | 48.03 | 19 | 11.25 |
| 5 | 97.84 | 10 | 77.26 | 15 | 42.39 | 20 | 10.30 |
| Mean | 100 | Mean | 74.11 | Mean | 42.62 | Mean | 19.03 |
| WV-20170/WV-36807 | | | | | | WV-38708/WV-36807 | |
| 0.6 mg/kg | | 2 mg/kg | | 6 mg/kg | | 0.6 mg/kg | |
| | % remaining | | % remaining | | % remaining | | % remaining |
| animal | of mTTR | animal | of mTTR | animal | of mTTR | animal | of mTTR |
| No. | mRNA | No. | mRNA | No. | mRNA | No. | mRNA |
| 21 | 59.04 | 26 | 27.62 | 31 | 8.38 | 36 | 74.11 |
| 22 | 64.79 | 27 | 24.76 | 32 | 9.90 | 37 | 51.80 |
| 23 | 57.41 | 28 | 39.43 | 33 | 4.61 | 38 | 75.30 |
| 24 | 63.78 | 29 | 33.50 | 34 | 6.79 | 39 | 58.13 |
| 25 | 61.99 | 30 | 28.70 | 35 | 14.13 | 40 | 75.99 |
| Mean | 61.40 | Mean | 30.80 | Mean | 8.76 | Mean | 67.07 |
| WV-38708/WV-36807 | | | | | | | |
| 2 mg/kg | | 6 mg/kg | | | | | |
| | % remainng | | % remaining | | | | |
| animal | of mTTR | animal | of mTTR | | | | |
| No. | mRNA | No. | mRNA | | | | |
| 41 | 14.89 | 46 | 3.01 | | | | |
| 42 | 12.19 | 47 | 5.27 | | | | |
| 43 | 19.18 | 48 | 3.76 | | | | |
| 44 | 23.48 | 49 | 2.98 | | | | |
| 45 | 17.54 | 50 | 3.94 | | | | |
| Mean | 17.46 | Mean | 3.79 | | | | |

Table 14. shows the accumulation of antisense strand in liver tissue. N=5. N.D.: Not determined.

TABLE 14

| | | WV-20167/WV-36860 | | | | | |
|---|---|---|---|---|---|---|---|
| | | | | 2 mg/kg | | | |
| | PBS | 0.6 mg/kg | | | % | 6 mg/kg | |
| | antisense | | antisense | | antisense | | antisense |
| | strand | | strand | | strand | | strand |
| animal | (μg/g of | animal | (μg/g of | animal | (μg/g of | animal | (μg/g of |
| No. | tissue) | No. | tissue) | No. | tissue) | No. | tissue) |
| 1 | 0.000 | 6 | 0.024 | 11 | 0.031 | 16 | 0.055 |
| 2 | 0.000 | 7 | 0.048 | 12 | 0.084 | 17 | 0.121 |
| 3 | 0.000 | 8 | 0.032 | 13 | 0.069 | 18 | 0.113 |
| 4 | 0.000 | 9 | 0.025 | 14 | 0.049 | 19 | 0.101 |
| 5 | 0.000 | 10 | 0.026 | 15 | 0.042 | 20 | 0.131 |
| Mean | 0.000 | Mean | 0.031 | Mean | 0.055 | Mean | 0.104 |
| WV-20170/WV-36807 | | | | | | WV-38708/WV-36807 | |
| 0.6 mg/kg | | 2 mg/kg | | 6 mg/kg | | 0.6 mg/kg | |
| | antisense | | antisense | | antisense | | antisense |
| | strand | | strand | | strand | | strand |
| animal | (μg/g of | animal | (μg/g of | animal | (μg/g of | animal | (μg/g of |
| No. | tissue) | No. | tissue) | No. | tissue) | No. | tissue) |
| 21 | 0.040 | 26 | 0.066 | 31 | 0.057 | 36 | 0.017 |
| 22 | 0.024 | 27 | 0.032 | 32 | 0.062 | 37 | 0.015 |
| 23 | 0.006 | 28 | 0.015 | 33 | 0.110 | 38 | 0.011 |
| 24 | 0.000 | 29 | 0.019 | 34 | 0.072 | 39 | 0.004 |
| 25 | 0.000 | 30 | 0.022 | 35 | 0.103 | 40 | 0.010 |
| Mean | 0.014 | Mean | 0.031 | Mean | 0.081 | Mean | 0.012 |
| WV-38708/WV-36807 | | | | | | | |
| 2 mg/kg | | 6 mg/kg | | | | | |
| | antisense | | antisense | | | | |
| | strand | | strand | | | | |
| animal | (μg/g of | animal | (μg/g of | | | | |
| No. | tissue) | No. | tissue) | | | | |
| 41 | 0.063 | 46 | 0.298 | | | | |
| 42 | 0.088 | 47 | 0.365 | | | | |
| 43 | 0.065 | 48 | 0.196 | | | | |
| 44 | 0.092 | 49 | 0.229 | | | | |
| 45 | 0.050 | 50 | 0.322 | | | | |
| Mean | 0.072 | Mean | 0.282 | | | | |

Table 15. shows Ago 2 loading of guide strand relative to miR-122. N=2.

TABLE 15

| | Ct: | Ct: miR- | Ct: | Ct: | Relative |
|---|---|---|---|---|---|
| | mTTR/ | 122/ | mTTR/ | miR- | mTTR/ |
| | Ago2 | Ago2 | IgG | 122/IgG | miR122 |
| PBS-1 | 39.07 | 16.86 | 38.67 | 27.78 | −0.02 |
| PBS-2 | 38.86 | 16.95 | 39.06 | 28.16 | 0.01 |
| WV-20167-1 | 36.57 | 16.94 | 38.63 | 28.58 | 0.34 |
| WV-20167-2 | 36.40 | 16.90 | 38.87 | 28.55 | 0.40 |
| WV-20170-1 | 34.56 | 16.63 | 38.67 | 27.49 | 1.35 |
| WV-20170-2 | 33.91 | 16.91 | 38.93 | 27.80 | 2.64 |
| WV-38708-1 | 31.10 | 16.48 | 36.70 | 28.02 | 13.94 |
| WV-38708-2 | 31.15 | 16.37 | 35.22 | 27.33 | 11.90 |

Table 15a. shows % mouse TTR protein remaining relative to PBS control. N=5. N.D.: Not determined.

TABLE 15a

| | PBS | | | | | |
|---|---|---|---|---|---|---|
| Day | animal1 | animal2 | animal3 | animal4 | animal5 | Mean |
| 1 | 91 | 114 | 104 | 91 | 100 | 100 |
| 8 | 103 | 120 | 98 | 91 | 87 | 100 |
| 15 | 91 | 100 | 112 | 103 | 93 | 100 |
| 22 | 94 | 107 | 91 | 102 | 106 | 100 |
| 29 | 93 | 101 | 87 | 108 | 111 | 100 |
| 36 | 87 | 102 | 95 | 112 | 104 | 100 |
| 43 | 98 | 95 | 95 | 101 | 112 | 100 |
| | WV-20167/WV-36860, 2 mg/kg | | | | | |
| Day | animal6 | animal7 | animal8 | animal9 | animal10 | Mean |
| 1 | 97 | 105 | 72 | 80 | 90 | 89 |
| 8 | 54 | 48 | 39 | 51 | 44 | 47 |
| 15 | 68 | 70 | 62 | 62 | 63 | 65 |
| 22 | 90 | 96 | 80 | 92 | 92 | 90 |
| 29 | 85 | 116 | 82 | 98 | 91 | 94 |
| 36 | 115 | 120 | 108 | 88 | 122 | 110 |
| 43 | 102 | 115 | 99 | 99 | 94 | 102 |
| | WV-20167/WV-36860, 6 mg/kg | | | | | |
| Day | animal11 | animal12 | animal13 | animal14 | animal15 | Mean |
| 1 | 92 | 102 | 109 | 119 | 92 | 103 |
| 8 | 18 | 18 | 19 | 18 | 16 | 18 |
| 15 | 33 | 38 | 39 | 30 | 34 | 35 |
| 22 | 57 | 78 | 72 | 55 | 66 | 66 |
| 29 | 72 | 80 | 85 | 71 | 81 | 78 |
| 36 | 90 | 119 | 104 | 96 | 100 | 102 |
| 43 | 94 | 98 | 115 | 105 | 106 | 104 |
| | WV-20170/WV-36807, 2 mg/kg | | | | | |
| Day | animal16 | animal17 | animal18 | animal19 | animal20 | Mean |
| 1 | 65 | 83 | 95 | 83 | 103 | 86 |
| 8 | 36 | 37 | 43 | 41 | 34 | 38 |
| 15 | 53 | 47 | 49 | 60 | 55 | 53 |
| 22 | 67 | 76 | 71 | 84 | 76 | 75 |
| 29 | 78 | 85 | 85 | 103 | 69 | 84 |
| 36 | 90 | 93 | 93 | 96 | 93 | 93 |
| 43 | 88 | 93 | 94 | 117 | 79 | 94 |
| | WV-20170/WV-36807, 6 mg/kg | | | | | |
| Day | animal21 | animal22 | animal23 | animal24 | animal25 | Mean |
| 1 | 108 | 129 | 100 | 105 | 107 | 110 |
| 8 | 9 | 11 | 4 | 8 | 15 | 10 |
| 15 | 17 | 19 | 9 | 19 | 29 | 19 |
| 22 | 39 | 40 | 18 | 39 | 58 | 39 |
| 29 | 67 | 69 | 41 | 56 | 79 | 62 |
| 36 | 90 | 94 | 61 | 84 | 110 | 88 |
| 43 | 108 | 114 | 80 | 90 | 107 | 100 |
| | WV-38708/WV-36807, 2 mg/kg | | | | | |
| Day | animal26 | animal27 | animal28 | animal29 | animal30 | Mean |
| 1 | 120 | 102 | 117 | 99 | 141 | 116 |
| 8 | 32 | 31 | 21 | 22 | 30 | 27 |
| 15 | 46 | 40 | 38 | 36 | 44 | 41 |
| 22 | 77 | 79 | 69 | 65 | 70 | 72 |
| 29 | 85 | 69 | 82 | 77 | 87 | 80 |
| 36 | 100 | 95 | 92 | 98 | 103 | 98 |
| 43 | 106 | 101 | 91 | 113 | 119 | 106 |
| | WV-38708/WV-36807, 6 mg/kg | | | | | |
| Day | animal31 | animal32 | animal33 | animal34 | animal35 | Mean |
| 1 | 102 | 103 | 107 | 105 | 117 | 107 |
| 8 | 7 | 4 | 4 | 5 | 7 | 6 |
| 15 | 13 | 9 | 7 | 11 | 12 | 10 |
| 22 | 27 | 20 | 19 | 27 | 30 | 25 |
| 29 | 48 | 38 | 40 | 52 | 63 | 48 |
| 36 | 77 | 66 | 66 | 72 | 83 | 73 |
| 43 | 82 | 89 | 77 | 115 | 94 | 91 |
| | WV-38706/WV-36807, 6 mg/kg | | | | | |
| Day | animal36 | animal37 | animal38 | animal39 | Animal40 | Mean |
| 1 | 114 | 129 | 142 | 104 | 111 | 120 |
| 8 | 123 | 93 | 100 | 106 | 102 | 105 |
| 15 | 127 | 111 | 119 | 104 | 100 | 112 |
| 22 | 130 | 100 | 101 | 103 | 109 | 109 |
| 29 | 121 | 91 | 126 | 102 | 105 | 109 |
| 36 | 117 | 135 | 108 | 106 | 109 | 115 |
| 43 | 120 | 114 | 120 | 132 | 102 | 117 |

### Abbreviation

1× reagent: TEA-3HF:TEA:H₂O: DMSO=5.0: 1.8: 15.5: 77.7 (v/v/v/v)
ADIH: 2-azido-1,3-dimethylimidazolium hexafluorophosphate
CMIMT: N-cyanomethylimidazolium triflate
CPG: controlled pore glass
DCM: dichloromethane, CH₂Cl₂
DIPEA: diisopropylethylamine
DMSO: dimethylsulfoxide
DMTr: 4,4′-dimethoxytrityl
GalNAc: N-acetylgalactosamine
HF: hydrogen fluoride
HATU: 1-[bis(dimethylamino)methylene]-1H-1,2,3-triazolo[4,5-b]pyridinium 3-oxide
hexafluorophosphate
IBN: isobutyronitrile
MeCN: acetonitrile
MeIm: N-methylimidazole
TCA: trichloroacetic acid
TEA: triethylamine
XH: xanthane hydride

### General Procedure for the Synthesis of Chiral-Oligos (25 μmol Scale):

The automated solid-phase synthesis of chiral-oligos was performed according to the cycles shown in Table 16 (regular amidite cycle, for PO linkages), Table 17 (regular amidite cycle, for stereo-random PS linkages), Table 18 (DPSE amidite cycle, for chiral PS linkages), and Table 19 (PSM amidite cycle, for chiral PN linkages).

TABLE 16
Regular Amidite Synthetic Cycle for PO linkages

| step | operation | reagents and solvent | volume | waiting time | |
|---|---|---|---|---|---|
| 1 | detritylation | 3% TCA/DCM | 10 mL | 65 | s |
| 2 | coupling | 0.2M monomer/20% IBN—MeCN | 0.5 mL | 8 | min |
| | | 0.5M CMIMT/MeCN | 1.0 mL | | |
| 3 | oxidation | 50 mM I₂/pyridine-H₂O (9:1, v/v) | 2.0 mL | 1 | min |
| 4 | cap-2 | 20% Ac₂O, 30% 2,6-lutidine/ | 1.0 mL | 45 | s |
| | | MeCN 20% MeIm/MeCN | 1.0 mL | | |

TABLE 17
Regular Amidite Synthetic Cycle for stereo-random PS linkages

| step | operation | reagents and solvent | volume | waiting time | |
|---|---|---|---|---|---|
| 1 | detritylation | 3% TCA/DCM | 10 mL | 65 | s |
| 2 | coupling | 0.2M monomer/20% IBN—MeCN | 0.5 mL | 8 | min |
| | | 0.5M CMIMT/MeCN | 1.0 mL | | |
| 3 | sulfurization | 0.2M XH/pyridine | 2.0 mL | 6 | min |
| 4 | cap-2 | 20% Ac₂O, 30% 2,6-lutidine/ | 1.0 mL | 45 | s |
| | | MeCN 20% MeIm/MeCN | 1.0 mL | | |

TABLE 18
DPSE Amidite Synthetic Cycle for chiral PS linkages

| step | operation | reagents and solvent | volume | waiting time | |
|---|---|---|---|---|---|
| 1 | detritylation | 3% TCA/DCM | 10 mL | 65 | s |
| 2 | coupling | 0.2M monomer/20% IBN—MeCN | 0.5 mL | 8 | min |
| | | 0.5M CMIMT/MeCN | 1.0 mL | | |
| 3 | cap-1 | 20% Ac₂O, 30% 2,6-lutidine/ | 2.0 mL | 2 | min |
| | | MeCN | | | |
| 4 | sulfurization | 0.2M XH/pyridine | 2.0 mL | 6 | min |
| 5 | cap-2 | 20% Ac₂O, 30% 2,6-lutidine/ | 1.0 mL | 45 | s |
| | | MeCN 20% MeIm/MeCN | 1.0 mL | | |

TABLE 19
PSM Amidite Synthetic Cycle for chiral PN linkages

| step | operation | reagents and solvent | volume | waiting time | |
|---|---|---|---|---|---|
| 1 | detritylation | 3% TCA/DCM | 10 mL | 65 | s |
| 2 | coupling | 0.2M monomer/20% IBN—MeCN | 0.5 mL | 8 | min |
| | | 0.5M CMIMT/MeCN | 1.0 mL | | |
| 3 | cap-1 | 20% Ac₂O, 30% 2,6-lutidine/ | 2.0 mL | 2 | min |
| | | MeCN | | | |
| 4 | imidation | 0.5M ADIH reagent/MeCN | 2.0 mL | 6 | min |
| 5 | cap-2 | 20% Ac₂O, 30% 2,6-lutidine/ | 1.0 mL | 45 | s |
| | | MeCN 20% MeIm/MeCN | 1.0 mL | | |

1. In some embodiments, preparations include one or more DPSE and/or PSM cycles

### General Procedure for the C&D Conditions (25 μmol Scale):

After completion of the synthesis, the CPG solid support was dried and transferred into 50 mL plastic tube. The CPG was treated with 1× reagent (2.5 mL; 100 μL/umol) for 3 h at 28° C., then added conc. NH₃ (5.0 mL; 200 μL/umol) for 24 h at 37° C. The reaction mixture was cooled to room temperature and the CPG was separated by membrane filtration, washed with 15 mL of H₂O. The crude material (filtrate) was analyzed by LTQ and RP-UPLC.

### General Procedure for the GalNAc Conjugation Conditions (1 μMol Scale):

Into a plastic tube, tri-GalNAc (2.0 eq.), HATU (1.9 eq.), and DIPEA (10 eq.) were dissolved in anhydrous MeCN (0.5 mL). The mixture was stirred for 10 min at room temperature, then the mixture was added into the amino-oligo (1 μmol) in H₂O (1 mL) and stirred for 1 h at 37° C. The reaction was monitored by LC-MS and RP-UPLC. After the reaction was completed, the resultant GalNAc-conjugated oligo was treated with conc. NH₃ (2 mL) for 1 h at 37° C. The solution was concentrated under vacuum to remove MeCN and conc. NH₃. The residue was then dissolved in H₂O (10 mL) for reversed phase purification.

### Example 8. Preparation of Modified 5′-Terminal Nucleotides and Phosphoramidites

Various technologies for preparing modified nucleotides and corresponding phosphoramidites to be incorporated into the 5′-terminus of oligonucleotides and oligonucleotide compositions are known and can be utilized in accordance with the present disclosure, including, for example, methods and reagents described in PCT/US21/33939, which is incorporated herein by reference in its entirety. Additional methods for preparing modified nucleotides are disclosed herein.

### Synthesis of WV-NU-230 and WV-NU-231

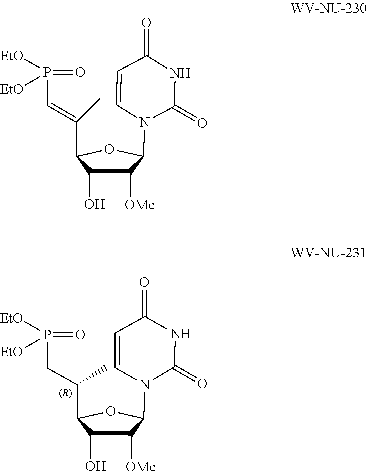

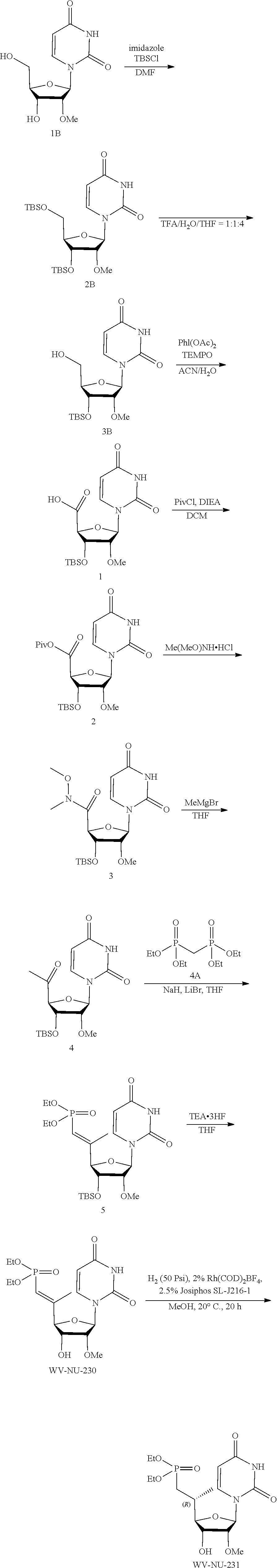

### 1. Preparation of Compound 2B

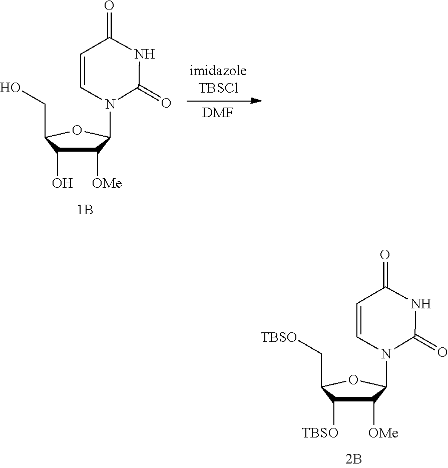

To a solution of compound 1B (100 g, 387.26 mmol, 1 eq.) in DMF (1600 mL) was added TBSCl (233.47 g, 1.55 mol, 189.81 mL, 4 eq.) and IMIDAZOLE (131.82 g, 1.94 mol, 5 eq.). The mixture was stirred at 20° C. for 12 hr. LCMS (ET28998-906-P1A1) showed the desired mass was detected. The reaction mixture was diluted with H₂O 2000 mL and extracted with ethyl acetate 3000 mL (1000 mL * 3). The combined organic layers were washed with brine 1000 mL, dried over Na₂SO₄, filtered and concentrated under reduced pressure to give a residue. The residue was purified by column chromatography (SiO₂, Petroleum ether/Ethyl acetate=1/0 to 0/1). TLC (Petroleum ether: Ethyl acetate=1:1, Rf=0.7). Compound 2B (188 g, crude) was obtained as a colorless oil.

TLC (Ethyl acetate: Methanol=1:1), R_{f}=0.7

LCMS (M−H⁺): 485.4

### 2. Preparation of Compound 3B

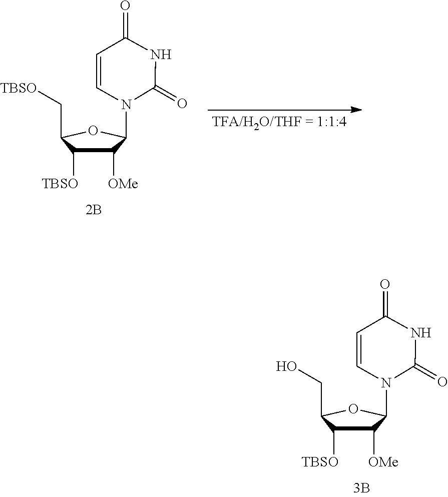

### For Two Batches:

To a stirred solution of compound 2B (94 g, 193.12 mmol, 1 eq.) in THF (800 mL) was added the mixture of TFA (200 mL) and H₂O (200 mL). The mixture was stirred at 0° C. for 5 hr. LCMS (ET28998-909-P1B1) showed the desired mass was detected. The reaction mixtures of two batches were combined and neutralized with saturated aqueous NaHCO₃ and extracted with ethyl acetate 1L*3. The combined organic layers were washed with brine 800*2 mL, dried over anhydrous Na₂SO₄ and evaporated at reduced pressure. The residue was purified by column chromatography (SiO₂, Petroleum ether/Ethyl acetate1=1/0 to 0/1). Compound 3B (70 g, 187.93 mmol, 48.66% yield) was obtained as a white solid.

LCMS (M−H⁺): 371.1

TLC (Petroleum ether: Ethyl acetate=1:1), Rf=0.3

### 3. Preparation of Compound 1

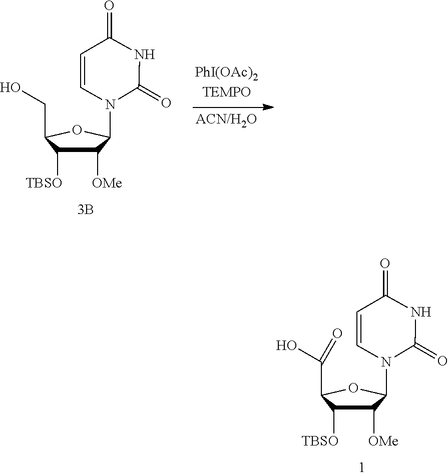

To a solution of Compound 3B (70 g, 187.93 mmol, 1 eq.) in the mixture of ACN (500 mL) and H2O (500 mL) was added PhI(OAc)₂ (133.17 g, 413.44 mmol, 2.2 eq.) and TEMPO (5.91 g, 37.59 mmol, 0.2 eq.). The mixture was stirred at 20° C. for 2 hr. LCMS (ET28998-916-P1A1) showed the desired mass was detected. The resulting mixture was concentrated then filtrated, and the solid was desired product. Compound 1(70 g, crude) was obtained as a white solid.

LCMS (M−H⁺): 385.2

### 4. Preparation of Compound 2

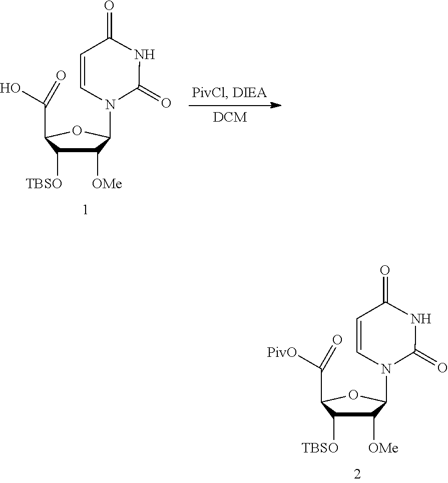

To a solution of compound 1 (70 g, 181.13 mmol, 1 eq.) in DCM (700 mL) was added DIEA (46.82 g, 362.25 mmol, 63.10 mL, 2 eq.) and 2,2-dimethylpropanoyl chloride (28.39 g, 235.46 mmol, 28.97 mL, 1.3 eq.). The mixture was stirred at −10˜0° C. for 1.5 hr. TLC (Petroleum ether: Ethyl acetate=1:1, R_{f}=0.3) indicated compound 1 was 10 consumed completely and one new spot formed. The crude product compound 2 (85.24 g, crude) in 700 mL DCM was used into the next step without further purification.

TLC (Petroleum ether: Ethyl acetate=1:1), Rf=0.3

### 5. Preparation of Compound 3

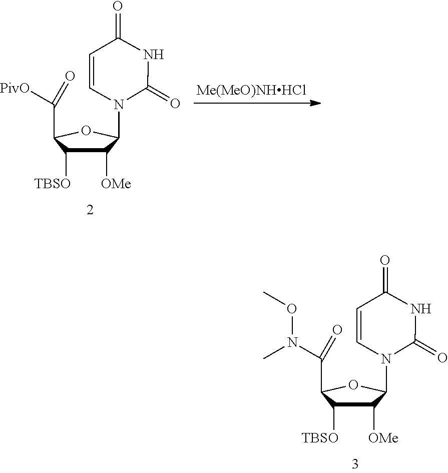

To a solution of Compound 2 (85.24 g, 181.14 mmol, 1 eq.) in DCM (ET28998-919) was added TEA (54.99 g, 543.41 mmol, 75.64 mL, 3 eq.) then added N-methoxymethanamine;hydrochloride (53.01 g, 543.41 mmol, 3 eq.). The mixture was stirred at 0° C. for 2 hr. LCMS (ET28998-920-P1A1) showed the desired mass was detected. The resulting mixture was washed with HCl (1M, 800 mL *2) and then aqueous NaHCO₃ (600 mL*2). The combined organic layers were dried over anhydrous Na₂SO₄, filtered and concentrated to get the product as a crude white solid. The residue was purified by column chromatography (SiO₂, Petroleum ether/Ethyl acetate=1/0 to 0:1). TLC (Petroleum ether: Ethyl acetate=0:1, Rf=0.7). Compound 3 (30 g, 69.84 mmol, 38.56% yield) was obtained as a white solid.

LCMS (M−H⁺): 428.3

TLC (Petroleum ether: Ethyl acetate=0:1), Rf=0.7

### 6. Preparation of Compound 4

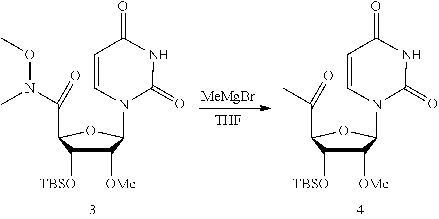

To a solution of compound 3 (30 g, 69.84 mmol, 1 eq.) in THF (250 mL) was added MeMgBr (3 M, 46.56 mL, 2 eq.). The mixture was stirred at 0° C. for 1.5 hr. TLC (Petroleum ether: Ethyl acetate=0:1, Rf=0.8) indicated compound 3 was consumed completely and new spot formed. The resulting mixture was poured into sat. NH₄Cl aq. (200 mL) under stirring, extracted with EtOAc (250 mL*3). The combined organic layers were dried over anhydrous Na₂SO₄, filtered and concentrated to give a crude. The residue was purified by column chromatography (SiO₂, Petroleum ether/Ethyl acetate=1/0 to 0:1). Compound 4 (20 g, 52.02 mmol, 74.48% yield) was obtained as a white solid.

LCMS (M−H⁺): 383.2

TLC (Petroleum ether: Ethyl acetate=0:1), Rf=0.8

### 7. Preparation of Compound 5

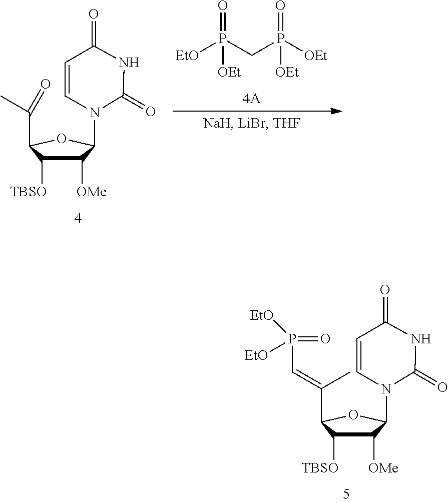

To a solution of NaH (4.58 g, 114.43 mmol, 60% purity, 4.4 eq.) in THF (50 mL) was added 1-[diethoxyphosphorylmethyl(ethoxy)phosphoryl]oxyethane (32.98 g, 114.43 mmol, 4.4 eq.) in THF (400 mL) at 0° C. The reaction mixture was warmed up to 20° C., and stirred for 1 hr. A solution of LiBr (9.94 g, 114.43 mmol, 2.87 mL, 4.4 eq.) in THF (100 mL) was added and the resultant slurry was stirred, and then cooled to 0° C. To the above mixture was added a solution of compound 4 (10 g, 26.01 mmol, 1 eq.) in THF (120 mL) at 0° C. The mixture was stirred at 0-20° C. for 12 hr. TLC (Petroleum ether: Ethyl acetate=2:1, Rf=0.1) indicated compound 4 was consumed completely and one new spot formed. The resulting mixture was diluted with water (500 mL), extracted with EtOAc (500 mL*3). The combined organic layers were washed with sat.brine (500 mL * 2), dried over anhydrous Na₂SO₄, filtered and concentrated to afford the crude. The crude was combined with ET28998-930-P1, then was purified by column chromatography (SiO₂, Petroleum ether/Ethyl acetate=1/0 to 0:1). Compound 5 (23 g, crude) was obtained as a colorless gum.

LCMS (M−H⁺): 517.1

TLC (Petroleum ether: Ethyl acetate=2:1), Rf=0.1

### 8. Preparation of Compound WV-NU-230

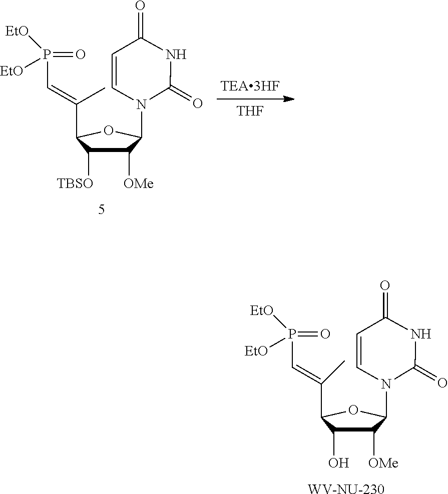

To a solution of compound 5 (23 g, 44.35 mmol, 1 eq.) in THF (250 mL) was added N,N-diethylethanamine;trihydrofluoride (57.20 g, 354.79 mmol, 57.83 mL, 8 eq.). The mixture was stirred at 40° C. for 6 hr. LCMS (ET28998-941-P1A2) showed compound 5 was consumed completely and one main peak with desired mass was detected. The reaction mixture was quenched by addition sat. NaHCO₃ aq. (200 mL) and NaHCO₃ solid to pH=7˜8 and stirred 20 min. The mixture was dried over Na₂SO₄, and concentrated under reduced pressure to give a residue. The residue was purified by column chromatography (SiO₂, Petroleum ether: Ethyl acetate=1/1 to 0/1 then Ethyl acetate: Methanol=1/0 to 3/1). TLC (Ethyl acetate: Methanol=10:1, Rf=0.3). Compound WV-NU-230 (16 g, 38.07 mmol, 85.83% yield, 96.20% purity) was obtained as a colorless gum.

¹H NMR (400 MHz, DMSO-d₆) δ=11.44 (s, 1H), 7.65 (d, J=8.1 Hz, 1H), 5.77 (d, J=4.4 Hz, 1H), 5.70-5.60 (m, 2H), 5.47 (d, J=7.0 Hz, 1H), 4.18-4.12 (m, 2H), 4.00-3.90 (m, 5H), 3.38 (s, 3H), 2.04 (d, J=2.8 Hz, 3H), 1.22 (dt, J=4.3, 7.0 Hz, 6H)

LCMS (M−H⁺): 403.1, purity: 96.20%

TLC (Ethyl acetate: Methanol=10:1), Rf=0.3

### 9. Preparation of Compound WV-NU-231

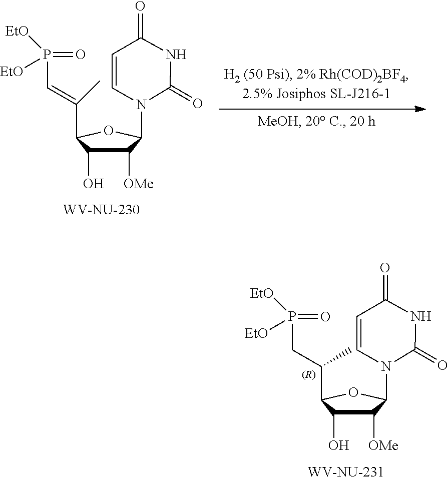

To a mixture of compound WV-NU-230 (13.5 g, 33.39 mmol, 1 eq.) in MeOH (400 mL) was added Josiphos SL-J216-1 (1.08 g, 66.77 mmol), (1Z,5Z)-cycloocta-1,5-diene;rhodium(1+);tetrafluoroborate (542.30 mg, 1.34 mmol, 0.04 eq.) and zinc;trifluoromethanesulfonate (4.85 g, 13.35 mmol, 0.4 eq.). And the system was stirred under H₂ (50 psi) for 20 hr at 20° C. LCMS (ET28998-952-P1A1) showed the desired mass was detected. The reaction mixture was filtered and concentrated under reduced pressure to give a residue. The crude product was purified by reversed-phase HPLC (0.1% NH3·H2O, DAC-150 Agela C18, 450 ml/min, 25-40% 30 min; 40-40% 30 min). Compound WV-NU-231 (10 g, 24.61 mmol, 73.71% yield, 100% purity) was obtained as a white solid.

¹H NMR (400 MHz, DMSO-d₆) δ=11.39 (s, 1H), 7.66-7.59 (m, 1H), 5.71 (d, J=5.0 Hz, 1H), 5.67 (dd, J=2.1, 8.0 Hz, 1H), 5.23-5.11 (m, 1H), 4.09-3.92 (m, 5H), 3.82 (t, J=5.5 Hz, 1H), 3.58 (t, J=5.9 Hz, 1H), 3.35 (s, 3H), 2.13-2.03 (m, 1H), 2.03-1.90 (m, 1H), 1.57 (ddd, J=9.8, 15.5, 17.4 Hz, 1H), 1.29-1.18 (m, 6H), 1.03 (d, J=6.6 Hz, 3H)

LCMS (M−H⁺): 405.2; purity: 100%

### Preparation of 3′-L-DPSE-2′-OMe-5′-PO(OEt)₂-Vinylphosphonate-U amidite (3′-L-DPSE-WV-NU-230)

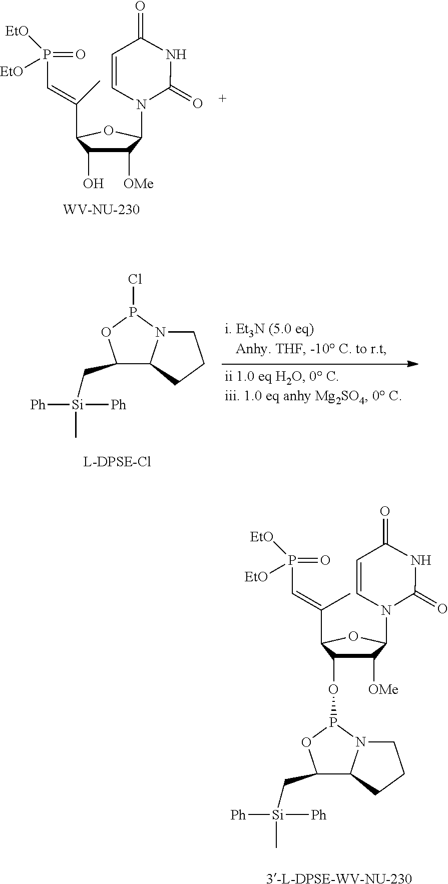

Nucleoside 2′-OMe-5′-(Me)-PO(OEt)₂-Vinylphosphonate-U, WV-NU-230 (1.60 g) was converted to 3′-L-DPSE-2′-OMe-5′-(Me)-PO(OEt)₂-Vinylphosphonate-U amidite (3′-L-DPSE-WV-NU-230) by general procedure and obtained (1.98 g, 67% yield) as an off-white solid.

LCMS: C₃₅H₄₇N₃O₉P₂Si (M−H⁻): 742.69

³¹P NMR (243 MHz, CDCl₃) δ=153.09, 17.24

¹H NMR (600 MHz, CDCl₃) δ=9.20 (s, 1H), 7.47-7.35 (m, 5H), 7.24 (q, J=6.2 Hz, 7H), 7.07 (d, J=8.1 Hz, 1H), 5.71-5.61 (m, 2H), 5.56 (t, J=2.7 Hz, 1H), 4.80 (q, J=6.8 Hz, 1H), 4.32 (dt, J=9.4, 6.1 Hz, 1H), 4.11 (d, J=6.3 Hz, 1H), 3.98 (tt, J=12.8, 7.9 Hz, 4H), 3.63 (t, J=4.8 Hz, 1H), 3.47-3.39 (m, 1H), 3.37-3.32 (m, 1H), 3.28 (d, J=2.0 Hz, 3H), 3.08 (qd, J=10.4, 4.2 Hz, 1H), 1.95 (d, J=3.2 Hz, 3H), 1.75 (dp, J=12.9, 5.1 Hz, 1H), 1.62-1.52 (m, 2H), 1.37 (dd, J=14.6, 6.6 Hz, 1H), 1.32-1.27 (m, 1H), 1.22 (t, J=6.9 Hz, 6H), 1.15 (td, J=8.6, 2.4 Hz, 1H), 0.55 (s, 3H).

¹³C NMR (151 MHz, CDCl₃) δ=163.12, 156.31, 156.26, 149.74, 141.09, 136.48, 135.94, 134.53, 134.51, 134.48, 134.36, 129.54, 129.47, 129.27, 128.14, 128.00, 127.96, 127.86, 113.72, 112.46, 102.90, 90.63, 85.36, 85.34, 85.21, 85.19, 81.33, 81.31, 79.55, 79.49, 77.27, 77.06.

### Preparation of 3′-L-DPSE-5′-(R)-Me-PO(OEt)₂Phosphonate-U amidite (3′-L-DPSE-WV-NU-231)

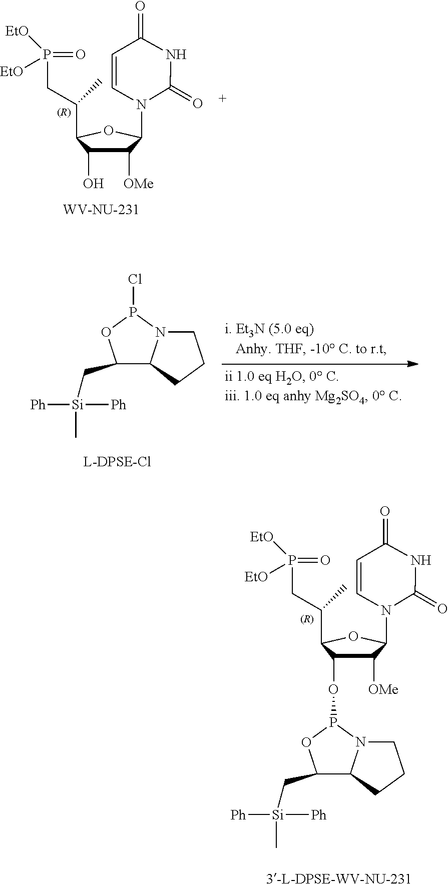

Nucleoside 2′-OMe-5′-(R)-Me-PO(OEt)₂-phosphonate-U, WV-NU-231 (5.0 g) was converted to 3′-L-DPSE-2′-OMe-5′-(R)-Me)-PO(OEt)₂-phosphonate-U amidite (3′-L-DPSE-WV-NU-231) by general procedure and obtained 7.9 g, 84% yield) as an off-white solid.

LCMS: C₃₅H₄₉N₃O₉P₂Si (M−H⁻): 744.85

¹H NMR (600 MHz, CDCl₃) δ ¹H NMR (600 MHz, CDCl₃) δ 8.48 (s, 1H), 7.64-7.47 (m, 5H), 7.38 (ddt, J=16.6, 8.8, 4.8 Hz, 5H), 7.27 (d, J=8.1 Hz, 1H), 5.77 (d, J=8.1 Hz, 1H), 5.72 (d, J=3.2 Hz, 1H), 4.95 (q, J=7.1 Hz, 1H), 4.21 (dt, J=9.7, 6.5 Hz, 1H), 4.18-4.04 (m, 3H), 3.89 (t, J=6.4 Hz, 1H), 3.69 (dd, J=5.7, 3.2 Hz, 1H), 3.57 (ddt, J=14.8, 10.5, 7.5 Hz, 1H), 3.46-3.39 (m, 1H), 3.27 (s, 3H), 3.18 (tdt, J=15.2, 10.6, 5.3 Hz, 1H), 2.34-2.22 (m, 1H), 2.10-1.97 (m, 2H), 1.85 (dtt, J=12.2, 8.1, 3.3 Hz, 1H), 1.69 (pd, J=16.4, 8.5 Hz, 4H), 1.51 (dd, J=14.5, 7.8 Hz, 1H), 1.34 (td, J=7.0, 2.2 Hz, 6H), 1.31-1.22 (m, 2H), 1.17 (d, J=6.8 Hz, 3H), 0.67 (s. 3H).

³¹P NMR (243 MHz, CDCl₃) δ=155.81, 30.61

¹³C NMR (151 MHz, CDCl₃) δ 162.64, 149.63, 140.01, 136.33, 136.11, 134.55, 134.51, 134.48, 134.46, 134.42, 129.58, 129.53, 128.09, 128.01, 127.98, 127.87, 102.66, 88.98, 85.60, 85.57, 85.45, 82.70, 79.07, 79.01, 71.21, 71.11, 67.33, 67.31, 61.60, 61.55, 61.51, 58.45, 46.86, 46.63, 30.53, 30.50, 29.72, 28.78, 26.96, 25.97, 25.95, 18.03, 18.01, 16.52, 16.51, 16.48, 16.46, 15.85, 15.82, −3.40.

### Preparation of 3′-D-DPSE-5′-(R)-Me-PO(OEt)₂Phosphonate-U amidite (3′-D-DPSE-WV-NU-231)

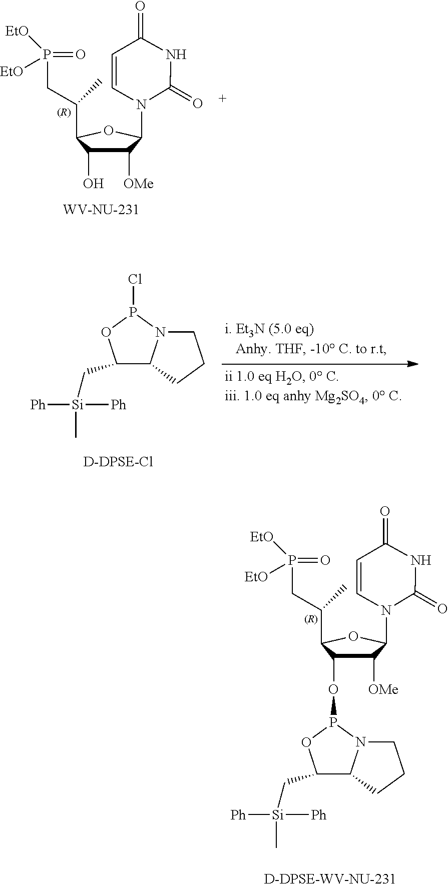

Nucleoside 2′-OMe-5′-(R)-Me-PO(OEt)₂-phosphonate-U, WV-NU-231 (2.5 g) was converted to 3′-D-DPSE-2′-OMe-5′-(R)-Me)-PO(OEt)₂-phosphonate-U amidite (3′-D-DPSE-WV-NU-231) by general procedure and obtained 2.8 g, 83% yield) as an off-white solid

LCMS: C₃₅H₄₉N₃O₉P₂Si (M−H⁻): 744.85

¹H NMR (600 MHz, CDCl₃) δ 9.24 (s, 1H), 7.54 (td, J=7.4, 1.7 Hz, 5H), 7.42-7.32 (m, 5H), 7.27 (d, J=8.1 Hz, 1H), 5.77 (d, J=8.1 Hz, 1H), 5.73 (d, J=3.1 Hz, 1H), 4.94 (td, J=7.5, 5.3 Hz, 1H), 4.21 (ddd, J=9.7, 7.2, 5.6 Hz, 1H), 4.11 (qdd, J=15.1, 6.9, 4.1 Hz, 4H), 3.88 (dd, J=7.3, 5.5 Hz, 1H), 3.69 (dd, J=5.7, 3.1 Hz, 1H), 3.56 (ddt, J=14.7, 10.6, 7.6 Hz, 1H), 3.41 (ddd, J=12.3, 9.8, 5.5 Hz, 1H), 3.27 (s, 3H), 3.18 (tdd, J=10.9, 8.8, 4.5 Hz, 1H), 2.29 (ttd, J=8.8, 6.4, 3.0 Hz, 1H), 2.06-1.97 (m, 1H), 1.84 (dp, J=12.7, 4.3 Hz, 1H), 1.68 (td, J=15.5, 7.5 Hz, 3H), 1.51 (dd, J=14.5, 7.8 Hz, 1H), 1.33 (td, J=7.0, 1.8 Hz, 6H), 1.29-1.24 (m, 1H), 1.17 (d, J=6.7 Hz, 3H), 0.68 (s, 3H).

³¹P NMR (243 MHz, CDCl₃) δ=155.73, 30.66

¹³C NMR (151 MHz, CDCl₃) δ 163.22, 149.88, 139.98, 136.34, 136.11, 134.55, 134.51, 134.49, 134.45, 129.57, 129.52, 128.00, 127.97, 127.85, 102.68, 88.93, 82.73, 79.06, 79.00, 71.24, 71.14, 67.32, 67.30, 61.60, 61.56, 61.51, 58.45, 46.85, 46.62, 30.52, 30.50, 29.69, 28.75, 26.96, 25.97, 25.95, 18.03, 18.01, 16.52, 16.50, 16.48, 16.46, 15.86, 15.84, −3.40.

### Selective Asymmetric Reduction of Methylketone Intermediate (6) to the Corresponding Hydroxymethyl (6A and 6B) Using Chiral Catalyst

### Preparation of Compound 6A

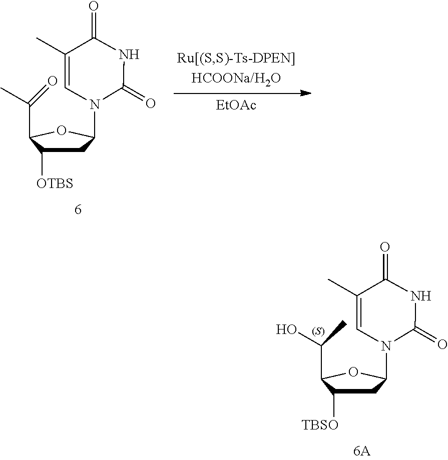

To a solution of compound 6 (46.00 g, 124.83 mmol) in the mixture of EtOAc (460.00 mL) and sodium formate (353.17 g, 5.19 mol) dissolved in Water (1.84 L), and then N−[(1S,2S)-2-amino-1,2-diphenyl-ethyl]-4-methyl-benzenesulfonamide; chlororuthenium;1-isopropyl-4-methyl-benzene (1.59 g, 2.50 mmol) was added. The resulting two-phase mixture was stirred for 12 h at 25° C. under N2. TLC showed the starting material was consumed. The mixture was extracted with EtOAc (500 mL*3). The combined organic was washed with brine (300 mL), dried over Na₂SO₄, filtered and concentrated to get the crude. The mixture was purified by MPLC (Petroleum ether/MTBE=10:1 to 1:1) to get compound 6A as a yellow oil (25.60 g, 57.53% yield).

¹H NMR (400 MHz, DMSO-d₆): δ=11.28 (s, 1H), 7.85 (s, 1H), 6.16 (t, J=6.8 Hz, 1H), 5.04 (d, J=4.6 Hz, 1H), 4.46-4.29 (m, 1H), 3.79 (br t, J=6.8 Hz, 1H), 3.59 (br s, 1H), 3.32 (s, 1H), 2.21-2.09 (m, 1H), 2.06-1.97 (m, 1H), 1.76 (s, 3H), 1.17-1.08 (m, 4H), 0.91-0.81 (m, 10H), 0.08 (s, 6H)

SFC: SFC purity: 98.6%

### Preparation of Compound 6B

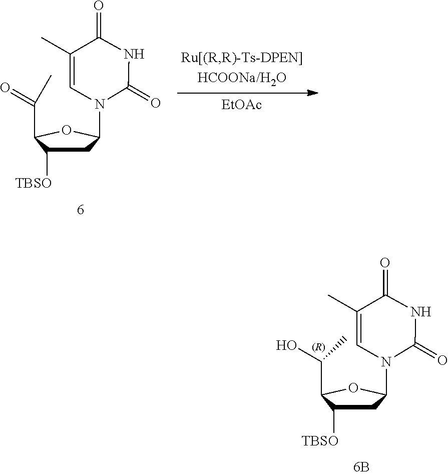

A 100 mL round-bottom flask equipped with a septum covered side arm was charged with [[(1R,2R)-2-amino-1,2-diphenyl-ethyl]-(p-tolylsulfonyl)amino]-chloro-ruthenium;i-isopropyl-4-methyl-benzene (34.53 mg, 54.27 μmol) and compound 6 (1.00 g, 2.71 mmol), and the system was flushed with nitrogen. A solution of sodium;formate;dihydrate (11.75 g, 112.89 mmol) in water (40.00 mL) was added, followed by EtOAc (10.00 mL). The resulting two-phase mixture was stirred for 12 h at 25° C. TLC showed the starting material was consumed. The mixture was extracted with EtOAc (50 mL*3). The combined organic was washed with brine (30 mL), dried over Na₂SO₄, filtered and concentrated to get the crude. The mixture was purified by MPLC (Petroleum ether/MTBE=10:1 to 1:1) to get compound 6B as a yellow oil (1.00 g, 99.50% yield).

¹H NMR (400 MHz, DMSO-d₆): δ=11.30 (s, 1H), 7.67 (s, 1H), 6.16 (dd, J=5.6, 8.7 Hz, 1H), 5.04 (d, J=5.1 Hz, 1H), 4.49 (br d, J=5.1 Hz, 1H), 3.86-3.66 (m, 1H), 3.55 (d, J=4.2 Hz, 1H), 2.50 (br s, 12H), 2.22-2.05 (m, 1H), 1.96 (br dd, J=5.6, 12.9 Hz, 1H), 1.77 (s, 3H), 1.11 (d, J=6.2 Hz, 4H), 0.94-0.81 (m, 10H), 0.09 (s, 6H);

HPLC: HPLC purity: 84.4%;

TLC (Petroleum ether/Ethyl acetate=1:1) Rf=0.37.

TABLE 20
Selective Asymmetric reduction of methylketone intermediates to the
corresponding hydroxymethyl intermediates using Chiral Catalyst (TLC clean, with
nearly quantitative conversion to alcohol)

| | | | | Selectivity |
|---|---|---|---|---|
| | | | | (Ratio R/S, |
| | | | Scale of | based on |
| Coumpound | | | methyl | HNMR or |
| ID | Structure | catalyst | ketone | SFC) |
| 5′-(S)-C-Me- 3′-OTBS-dT | 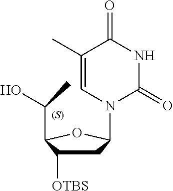 | RuCl(p-cymene)[(S,S)- Ts-DPEN] | 0.35 g | 13:100 |
| 5′-(R)-C-Me- 3′-OTBS-dT | 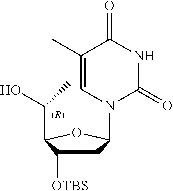 | RuCl(p-cymene)[(R,R)- Ts-DPEN] | 0.35 g | 100:3.7 |
| 5′-(S)-C-Me- 3′-OTBS-2′- OMe-U | 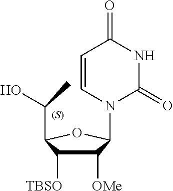 | RuCl(p-cymene)[(S,S)- Ts-DPEN] | 0.08 g | 17:100 |
| 5′-(R)-C-Me- 3′-OTBS-2′- OMe-U | 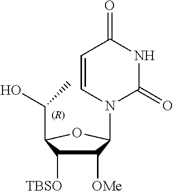 | RuCl(p-cymene)[(R,R)- Ts-DPEN] | 0.08 g | 100:3.8 |
| 5′-(S)-C-Me- 3′-OTBS-2′- F-dU | 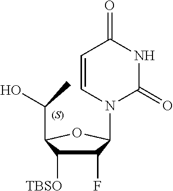 | RuCl(p-cymene)[(S,S)- Ts-DPEN] | 0.05 g | 58:100 |
| 5′-(R)-C-Me- 3′-OTBS-2′- F-dU | 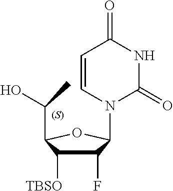 | RuCl(p-cymene)[(R,R)- Ts-DPEN] | 0.45 g | No S isomer was observed |
| 5′-(S)-C-Me- 3′-OTBS-2′- F-dA(Bz) | 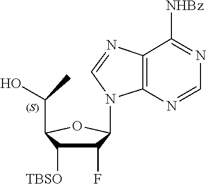 | RuCl(p-cymene)[(S,S)- Ts-DPEN] | 3.8 g | 17.8:100 |
| 5′-(R)-C-Me- 3′-OTBS-2′- F-dA(Bz) | 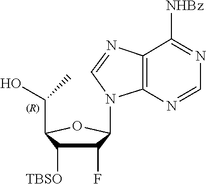 | RuCl(p-cymene)[(R,R)- Ts-DPEN] | 16.2 g | 96.5:3.5 by SFC |
| 5′-(S)-C-Me- 3′-OTBS-2′- F-dA(Bz) | 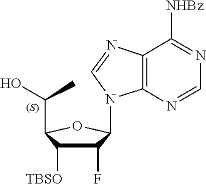 | RuCl(mesitylene)[(S,S)- Ts-DPEN] | 50 mg | 15:85 by SFC |
| 5′-(S)-C-Me- 3′-OTBS-2′- F-dA(Bz) | 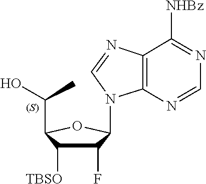 | RuCl(p-cymene)[(S,S)- Fsdpen] | 50 mg | 7.90:92.10 by SFC |
| 5′-(S)-C-Me- 3′-OTBS-2′- F-dA(Bz) | 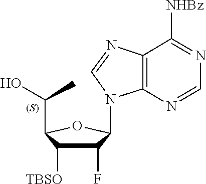 | RuCl2[(S)- xylbinap][(S,S)-dpen] | 50 mg | 86.86:11.95 by SFC |
| 5′-(S)-C-Me- 3′-OTBS-2′- OMe-A(Bz) | 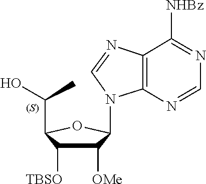 | RuCl(p-cymene)[(S,S)- Ts-DPEN] | 50 mg | 8.2:91.8 by SFC |
| 5′-(R)-C-Me- 3′-OTBS-2′- OMe-A(Bz) | 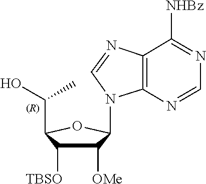 | RuCl(p-cymene)[(R,R)- Ts-DPEN] | 50 mg | No S isomer was observed by SFC |
| 5′-(S)-C-Me- 3′-OTBS-2′- F-dG(iBu) | 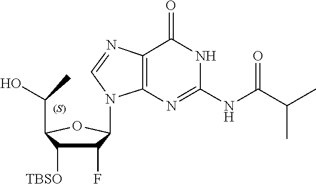 | RuCl(p-cymene)[(S,S)- Ts-DPEN] | 50 mg | 31.3:68.6 by SFC |
| 5′-(S)-C-Me- 3′-OTBS-2′- F-dG(iBu) | 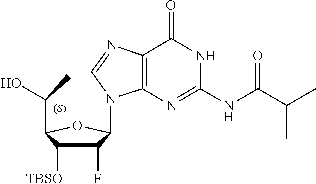 | RuCl(mesitylene)[(S,S)- Ts-DPEN] | 50 mg | 33.2:66.8 by SFC |
| 5′-(S)-C-Me- 3′-OTBS-2′- F-dG(iBu) | 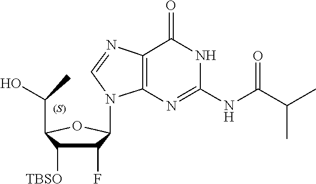 | RuCl(p-cymene)[(S,S)- Fsdpen] | 50 mg | 17.46:78.39 by SFC |
| 5′-(S)-C-Me- 3′-OTBS-2′- F-dG(iBu) | 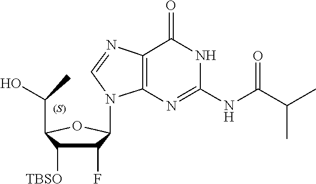 | RuCl2[(S)- xylbinap][(S,S)-dpen] | 50 mg | 28.92:71.08 by SFC |
| 5′-(R)-C-Me- 3′-OTBS-2′- F-dG(iBu) | 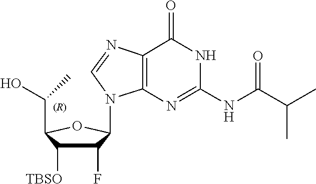 | RuCl(p-cymene)[(R,R)- Ts-DPEN] | 50 mg | 99.76:0.24 by SFC |
| 5′-(S)-C-Me- 3′-OTBS-2′- OMe-G(iBu) | 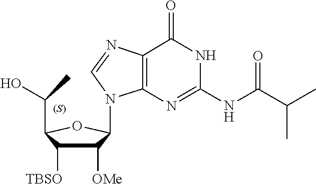 | RuCl(p-cymene)[(S,S)- Ts-DPEN] | 35 mg 50 mg | 18.19:81.81 12.67:87.33 by SFC |
| 5′-(R)-C-Me- 3′-OTBS-2′- OMe-G(iBu) | 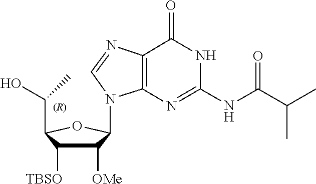 | RuCl(p-cymene)[(R,R)- Ts-DPEN] | 35 mg 50 mg | 98.78: 1.25 99.11: 0.89 by SFC |
| 5′-(S)-C-Me- 3′-OTBS-2′- OMe-G(iBu) | 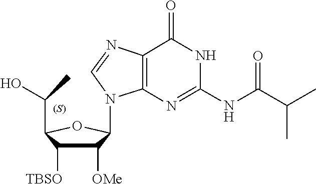 | RuCl2[(S)- xylbinap][(S,S)-dpen]] | 50 mg | 94.26:5.74 by SFC |
| 5′-(S)-C-Me- 3′-OTBS-2′- OMe-G(iBu) | 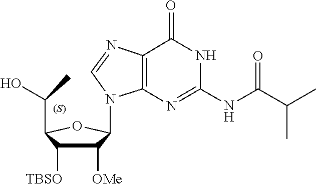 | RuCl(p-cymene)[(S,S)- Fsdpen] | 50 mg | 10:90 by SFC |
| 5′-(S)-C-Me- 3′-OTBS-2′- OMe-G(iBu, CE) | 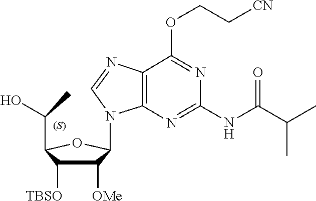 | RuCl(p-cymene)[(S,S)- Ts-DPEN] | 50 mg | 11:89 by SFC |
| 5′-(R)-C-Me- 3′-OTBS-2′- OMe-G(iBu, CE) | 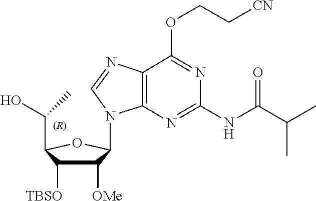 | RuCl(p-cymene)[(R,R)- Ts-DPEN] | 50 mg | 86.7:13.3 by SFC |

### Example 9. Provided Oligonucleotides and Compositions can Effectively Knockdown Mouse Transthyretin (mTTR) In Vitro

Various siRNAs for mouse TTR were designed and constructed. A number of siRNAs were tested in vitro in mouse primary hepatocytes at one or a range of concentrations. Some siRNAs were also tested in mice (e.g., C57BL6 wild type mice).

Example protocol for in vitro determination of siRNA activity: For determination of siRNAs activity, siRNAs at specific concentration were gymnotically delivered to mouse primary hepatocytes plated at 96-well plates, with 10,000 cells/well. Following 48 hours treatment, total RNA was extracted using SV96 Total RNA Isolation kit (Promega). cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). For mouse TTR mRNA, the following qPCR assay were utilized: IDT Taqman qPCR assay ID Mm.PT.58.1 1922308. Mouse HPRT was used as normalizer (Forward 5′CAAACTTTGCTTTCCCTGGTT3′, Reverse 5′TGGCCTGTATCCAACACTTC3′, Probe 5′/5HEX(ACCAGCAAG/Zen/CTTGCAACCTTAACC/31ABkFQ/3′. mRNA knockdown levels were calculated as % mRNA remaining relative to mock treatment.

Table 21 shows % mouse TTR mRNA remaining (at 500 pM siRNA treatment) relative to mouse HPRT control. N=3. N.D.: Not determined.

TABLE 21

| | | 500 pM | | | |
|---|---|---|---|---|---|
| | | % remaining | % remaining | % remaining | |
| | | mRNA (mTTR/ | mRNA (mTTR/ | mRNA (mTTR/ | |
| Guide | Passenger | mHPRT)-1 | mHPRT)-2 | mHPRT)-3 | Mean |
| WV-49900 | WV-49901 | 120.90 | 114.37 | 101.46 | 112.24 |
| WV-20167 | WV-40362 | 59.82 | 29.16 | 23.90 | 37.63 |
| WV-38082 | WV-40362 | 73.90 | 51.03 | 46.83 | 57.25 |
| WV-38083 | WV-40363 | 53.73 | 57.11 | 48.97 | 53.27 |
| WV-38087 | WV-40363 | 39.03 | 43.19 | 28.10 | 36.77 |
| WV-38088 | WV-40363 | 59.68 | 47.30 | 42.19 | 49.72 |
| WV-38089 | WV-40363 | 59.53 | 44.21 | 46.44 | 50.06 |
| WV-38090 | WV-40363 | 75.52 | 64.54 | 62.35 | 67.47 |
| WV-38091 | WV-40363 | 65.48 | 59.13 | 58.71 | 61.11 |
| WV-38092 | WV-40363 | 64.97 | 60.19 | 57.67 | 60.94 |
| WV-38093 | WV-40363 | 64.59 | 31.88 | 59.36 | 51.95 |
| WV-38094 | WV-40363 | 46.50 | 51.48 | 37.42 | 45.13 |
| WV-38095 | WV-40363 | 83.34 | 69.59 | 55.00 | 69.31 |
| WV-38096 | WV-40363 | 73.19 | 58.01 | 51.31 | 60.84 |
| WV-38097 | WV-40363 | 85.43 | 57.53 | 62.26 | 68.41 |
| WV-38098 | WV-40363 | 79.94 | 61.60 | 53.10 | 64.88 |
| WV-38099 | WV-40363 | 77.02 | 64.19 | 71.05 | 70.75 |
| WV-38100 | WV-40363 | 65.21 | 62.32 | 55.15 | 60.89 |
| WV-38101 | WV-40363 | 58.00 | 61.54 | 50.00 | 56.51 |
| WV-38102 | WV-40363 | 36.11 | 36.05 | 36.22 | 36.13 |
| WV-38103 | WV-40363 | 84.29 | 74.41 | 65.59 | 74.76 |
| WV-38104 | WV-40363 | 73.03 | 61.01 | 58.95 | 64.33 |
| WV-38105 | WV-40363 | 81.59 | 51.47 | 56.04 | 63.03 |
| WV-38106 | WV-40363 | 82.19 | 59.25 | 46.87 | 62.77 |
| WV-38107 | WV-40363 | 49.87 | 34.92 | 19.30 | 34.70 |
| WV-38108 | WV-40363 | 72.81 | 71.75 | 56.98 | 67.18 |
| WV-38109 | WV-40363 | 55.56 | 45.56 | 29.80 | 43.64 |
| WV-38110 | WV-40363 | 53.71 | 50.18 | 43.35 | 49.08 |
| WV-38111 | WV-40363 | 87.18 | 70.09 | 61.13 | 72.80 |
| WV-38112 | WV-40363 | 75.97 | 64.42 | 47.73 | 62.71 |
| WV-38113 | WV-40363 | 83.21 | 64.31 | 50.24 | 65.92 |
| WV-38114 | WV-40363 | 69.76 | 52.97 | 40.53 | 54.42 |
| WV-38115 | WV-40363 | 60.74 | 57.99 | 49.54 | 56.09 |
| WV-38116 | WV-40363 | 71.99 | 51.19 | 49.65 | 57.61 |
| WV-38117 | WV-40363 | 73.94 | 55.86 | 30.48 | 53.43 |
| WV-38118 | WV-40363 | 62.81 | 58.61 | 53.42 | 58.28 |
| WV-38119 | WV-40363 | 72.08 | 59.52 | 51.76 | 61.12 |
| WV-38120 | WV-40363 | 69.88 | 62.10 | 50.50 | 60.83 |
| WV-38121 | WV-40363 | 79.39 | 53.64 | 51.87 | 61.63 |
| WV-38122 | WV-40363 | 68.70 | 54.47 | 44.11 | 55.76 |
| WV-38123 | WV-40363 | 82.10 | 49.07 | 45.98 | 59.05 |
| WV-38124 | WV-40363 | 68.99 | 57.03 | 48.45 | 58.16 |
| WV-38125 | WV-40363 | 74.20 | 55.91 | 36.03 | 55.38 |
| WV-38126 | WV-40363 | 62.69 | 63.94 | 53.84 | 60.16 |
| WV-38127 | WV-40363 | 59.27 | 52.65 | 44.37 | 52.10 |
| WV-38128 | WV-40363 | 76.51 | 56.68 | 46.62 | 59.94 |
| WV-38129 | WV-40363 | 73.04 | 54.19 | 54.27 | 60.50 |
| WV-38130 | WV-40363 | 73.30 | 54.69 | 58.48 | 62.16 |
| WV-38131 | WV-40363 | 81.34 | 58.73 | 45.15 | 61.74 |
| WV-38132 | WV-40363 | 77.89 | 48.98 | 43.97 | 56.95 |
| WV-38133 | WV-40363 | 75.61 | 60.42 | 24.69 | 53.58 |
| WV-38134 | WV-40363 | 58.30 | 62.92 | 46.05 | 55.76 |
| WV-38135 | WV-40363 | 85.60 | 82.02 | 51.16 | 72.92 |
| WV-38136 | WV-40363 | 58.81 | 55.17 | 46.51 | 53.50 |
| WV-38137 | WV-40363 | 76.56 | 57.07 | 42.31 | 58.65 |
| WV-38138 | WV-40363 | 77.34 | 57.88 | 58.21 | 64.48 |
| WV-38139 | WV-40363 | 77.20 | 60.85 | 42.46 | 60.17 |
| WV-38140 | WV-40363 | N.D. | N.D. | N.D. | N.D. |
| WV-38141 | WV-40363 | 71.95 | 47.17 | 21.88 | 47.00 |
| WV-38142 | WV-40363 | 46.58 | 57.73 | 46.63 | 50.31 |
| WV-38143 | WV-40363 | 81.50 | 75.43 | 53.16 | 70.03 |
| WV-38144 | WV-40363 | 66.01 | 50.97 | 43.47 | 53.48 |
| WV-38145 | WV-40363 | 63.60 | 57.09 | 46.72 | 55.80 |
| WV-38146 | WV-40363 | 69.89 | 64.70 | 37.16 | 57.25 |

Table 22 shows % mouse TTR mRNA remaining (at 1000 pM siRNA treatment) relative to mouse HPRT control. N=3. N.D.: Not determined.

TABLE 22

| | | 1000 pM | | | |
|---|---|---|---|---|---|
| | | % remaining | % remaining | % remaining | |
| | | mRNA (mTTR/ | mRNA (mTTR/ | mRNA (mTTR/ | |
| Guide | Passenger | mHPRT)-1 | mHPRT)-2 | mHPRT)-3 | Mean |
| WV-49900 | WV-49901 | 95.37 | 101.03 | 108.67 | 101.69 |
| WV-20167 | WV-40362 | 34.89 | 25.17 | 22.80 | 27.62 |
| WV-37236 | WV-40362 | 61.56 | 57.41 | 27.55 | 48.84 |
| WV-20170 | WV-40363 | 27.71 | 25.33 | 24.42 | 25.82 |
| WV-20171 | WV-40363 | 22.39 | 13.94 | 10.64 | 15.66 |
| WV-36980 | WV-40363 | 63.46 | 52.97 | 52.93 | 56.46 |
| WV-36981 | WV-40363 | 51.72 | 54.10 | 46.52 | 50.78 |
| WV-36982 | WV-40363 | 52.98 | 55.30 | 61.60 | 56.62 |
| WV-36983 | WV-40363 | 52.24 | 48.56 | 67.47 | 56.09 |
| WV-36984 | WV-40363 | 57.66 | 38.95 | 48.97 | 48.53 |
| WV-36985 | WV-40363 | 47.42 | 52.51 | 51.15 | 50.36 |
| WV-36986 | WV-40363 | 53.05 | 49.97 | 40.91 | 47.97 |
| WV-36987 | WV-40363 | 47.88 | 50.92 | 34.27 | 44.36 |
| WV-36988 | WV-40363 | 60.66 | 74.85 | 72.99 | 69.50 |
| WV-36989 | WV-40363 | 54.77 | 66.07 | 78.98 | 66.61 |
| WV-36990 | WV-40363 | 75.51 | 66.73 | 95.83 | 79.36 |
| WV-36991 | WV-40363 | 70.41 | 59.47 | 82.83 | 70.90 |
| WV-36992 | WV-40363 | 64.38 | 55.96 | 77.11 | 65.81 |
| WV-36993 | WV-40363 | 53.94 | 69.33 | 77.10 | 66.79 |
| WV-36994 | WV-40363 | 63.87 | 62.53 | 75.65 | 67.35 |
| WV-36995 | WV-40363 | 55.14 | 50.25 | 65.65 | 57.01 |
| WV-36996 | WV-40363 | 52.01 | 50.62 | 59.48 | 54.04 |
| WV-36997 | WV-40363 | 55.61 | 46.59 | 70.46 | 57.55 |
| WV-36998 | WV-40363 | 54.16 | 45.99 | 71.40 | 57.18 |
| WV-36999 | WV-40363 | 54.52 | 40.47 | 49.10 | 48.03 |
| WV-37000 | WV-40363 | 49.95 | 46.55 | 51.85 | 49.45 |
| WV-37001 | WV-40363 | 49.12 | 60.13 | 57.61 | 55.62 |
| WV-37002 | WV-40363 | 48.04 | 54.86 | 55.66 | 52.85 |
| WV-37003 | WV-40363 | 56.98 | 53.70 | 69.24 | 59.97 |
| WV-37004 | WV-40363 | 61.31 | 65.35 | 87.88 | 71.51 |
| WV-37005 | WV-40363 | 74.13 | 66.10 | 101.18 | 80.47 |
| WV-37006 | WV-40363 | 71.64 | 79.38 | 108.73 | 86.58 |
| WV-37007 | WV-40363 | 62.94 | 61.82 | 71.70 | 65.49 |
| WV-37008 | WV-40363 | 66.13 | 65.89 | 85.86 | 72.63 |
| WV-37009 | WV-40363 | 54.52 | 60.97 | 65.85 | 60.45 |
| WV-37010 | WV-40363 | 66.76 | 82.01 | 79.83 | 76.20 |
| WV-37011 | WV-40363 | 56.38 | 64.17 | 61.51 | 60.68 |
| WV-37012 | WV-40363 | 54.15 | 53.96 | 66.17 | 58.09 |
| WV-37013 | WV-40363 | 67.01 | 49.24 | 76.78 | 64.34 |
| WV-37014 | WV-40363 | 62.94 | 59.65 | 74.11 | 65.57 |
| WV-37015 | WV-40363 | 63.01 | 50.82 | 66.97 | 60.27 |
| WV-37016 | WV-40363 | 59.09 | 52.69 | 62.89 | 58.22 |
| WV-37017 | WV-40363 | 53.56 | 50.31 | 51.38 | 51.75 |
| WV-37018 | WV-40363 | 50.12 | 48.33 | 35.05 | 44.50 |
| WV-37019 | WV-40363 | 60.09 | 72.34 | 83.10 | 71.84 |
| WV-37020 | WV-40363 | 66.99 | 68.44 | 69.44 | 68.29 |
| WV-37021 | WV-40363 | 62.63 | 67.19 | 101.08 | 76.97 |
| WV-37022 | WV-40363 | 75.03 | 63.39 | 103.87 | 80.76 |
| WV-37023 | WV-40363 | 64.36 | 61.35 | 43.54 | 56.42 |
| WV-37024 | WV-40363 | 64.50 | 67.48 | 87.74 | 73.24 |
| WV-37025 | WV-40363 | 60.76 | 64.57 | 44.40 | 56.58 |
| WV-37026 | WV-40363 | 65.71 | 69.16 | 62.06 | 65.64 |
| WV-37027 | WV-40363 | 65.31 | 61.27 | 66.40 | 64.33 |
| WV-37028 | WV-40363 | 63.70 | 58.59 | 73.18 | 65.16 |
| WV-37029 | WV-40363 | 65.71 | 53.51 | 71.40 | 63.54 |
| WV-37030 | WV-40363 | 64.34 | 62.04 | 75.72 | 67.37 |
| WV-37031 | WV-40363 | 70.65 | 48.90 | 65.65 | 61.73 |
| WV-37032 | WV-40363 | 66.89 | 59.73 | 72.74 | 66.45 |
| WV-37033 | WV-40363 | 70.19 | 61.34 | 52.67 | 61.40 |
| WV-37034 | WV-40363 | 63.25 | 66.82 | 49.06 | 59.71 |
| WV-37035 | WV-40363 | 71.91 | 77.02 | 78.94 | 75.95 |
| WV-37036 | WV-40363 | 81.57 | 81.06 | 114.02 | 92.22 |
| WV-37037 | WV-40363 | 76.80 | 78.27 | 104.57 | 86.55 |
| WV-37038 | WV-40363 | 83.63 | 77.74 | N.D. | 80.69 |
| WV-37039 | WV-40363 | 87.33 | 72.36 | 78.02 | 79.24 |
| WV-37040 | WV-40363 | 78.91 | 62.83 | 106.89 | 82.88 |
| WV-37041 | WV-40363 | 75.13 | 77.56 | 92.26 | 81.65 |
| WV-37042 | WV-40363 | 77.87 | 72.59 | 64.49 | 71.65 |
| WV-37043 | WV-40363 | 59.88 | 62.39 | 41.31 | 54.53 |
| WV-37044 | WV-40363 | 59.28 | 56.73 | 67.16 | 61.06 |
| WV-37045 | WV-40363 | 63.79 | 53.40 | 77.62 | 64.93 |
| WV-37046 | WV-40363 | 69.88 | 52.44 | 62.98 | 61.77 |
| WV-37047 | WV-40363 | 67.02 | 60.15 | 53.36 | 60.18 |
| WV-37048 | WV-40363 | 56.29 | 43.39 | 54.35 | 51.34 |
| WV-37049 | WV-40363 | 54.74 | 50.80 | 35.31 | 46.95 |
| WV-37050 | WV-40363 | 58.86 | 50.76 | 37.26 | 48.96 |
| WV-37051 | WV-40363 | 59.45 | 61.74 | 46.74 | 55.97 |
| WV-37052 | WV-40363 | 71.45 | 64.11 | 67.47 | 67.68 |
| WV-37053 | WV-40363 | 76.67 | 59.85 | 88.02 | 74.85 |
| WV-37054 | WV-40363 | 83.63 | 65.28 | 78.83 | 75.91 |
| WV-37055 | WV-40363 | 67.58 | 65.63 | 82.96 | 72.05 |
| WV-37056 | WV-40363 | 73.16 | 55.23 | 74.02 | 67.47 |
| WV-37057 | WV-40363 | 76.89 | 60.29 | 50.44 | 62.54 |
| WV-37058 | WV-40363 | 75.63 | 62.13 | 57.65 | 65.13 |
| WV-37059 | WV-40363 | 58.11 | 55.87 | 39.97 | 51.31 |
| WV-37060 | WV-40363 | 56.41 | 57.62 | 65.15 | 59.72 |
| WV-37061 | WV-40363 | 76.32 | 62.43 | 52.35 | 63.70 |
| WV-37062 | WV-40363 | 70.13 | 58.31 | 67.32 | 65.25 |
| WV-37063 | WV-40363 | 68.62 | 55.62 | 56.01 | 60.08 |
| WV-37064 | WV-40363 | 64.47 | 45.83 | 53.15 | 54.48 |
| WV-37065 | WV-40363 | 71.72 | 48.67 | 36.39 | 52.26 |
| WV-20167 | WV-40362 | 37.19 | 16.85 | 20.75 | 24.93 |
| WV-37236 | WV-40362 | 51.32 | 4.37 | 50.75 | 35.48 |
| WV-20170 | WV-40363 | 28.43 | 16.24 | 15.22 | 19.96 |
| WV-20171 | WV-40363 | 24.24 | 16.75 | 10.96 | 17.31 |
| WV-37066 | WV-40363 | 72.94 | 58.81 | 40.92 | 57.56 |
| WV-37067 | WV-40363 | 66.96 | 65.85 | 60.36 | 64.39 |
| WV-37068 | WV-40363 | 73.66 | 72.30 | 71.41 | 72.46 |
| WV-37069 | WV-40363 | 87.13 | 89.14 | 50.75 | 75.67 |
| WV-37070 | WV-40363 | 84.22 | 105.77 | 63.05 | 84.34 |
| WV-37071 | WV-40363 | 62.83 | 73.71 | 41.78 | 59.44 |
| WV-37072 | WV-40363 | 75.03 | 77.78 | 52.60 | 68.47 |
| WV-37073 | WV-40363 | 85.10 | 55.41 | 65.89 | 68.80 |
| WV-37074 | WV-40363 | 88.38 | 71.51 | 69.74 | 76.54 |
| WV-37075 | WV-40363 | 68.79 | 67.16 | 50.63 | 62.19 |
| WV-37076 | WV-40363 | 62.02 | 55.05 | 52.91 | 56.66 |
| WV-37077 | WV-40363 | 76.72 | 65.53 | 32.37 | 58.21 |
| WV-37078 | WV-40363 | 78.74 | 79.92 | 37.11 | 65.25 |
| WV-37079 | WV-40363 | 65.36 | 78.81 | 29.79 | 57.99 |
| WV-37080 | WV-40363 | 63.19 | 55.56 | 36.07 | 51.60 |
| WV-37081 | WV-40363 | 65.59 | 61.12 | 39.69 | 55.47 |
| WV-37082 | WV-40363 | 72.40 | 59.86 | 52.69 | 61.65 |
| WV-37083 | WV-40363 | 85.98 | 78.86 | 64.36 | 76.40 |
| WV-37084 | WV-40363 | 75.67 | 93.25 | 61.72 | 76.88 |
| WV-37085 | WV-40363 | 86.04 | 107.79 | 50.80 | 81.54 |
| WV-37086 | WV-40363 | 95.36 | 111.85 | 58.16 | 88.46 |
| WV-37087 | WV-40363 | 89.45 | 103.95 | 52.61 | 82.00 |
| WV-37088 | WV-40363 | 87.91 | 76.75 | 64.01 | 76.22 |
| WV-37089 | WV-40363 | 100.36 | 92.30 | 65.95 | 86.20 |
| WV-37090 | WV-40363 | 86.67 | 88.29 | 65.24 | 80.06 |
| WV-37091 | WV-40363 | 74.12 | 66.72 | 51.23 | 64.02 |
| WV-37092 | WV-40363 | 60.39 | 57.35 | 46.99 | 54.91 |
| WV-37093 | WV-40363 | 83.99 | 88.96 | 44.62 | 72.52 |
| WV-37094 | WV-40363 | 79.91 | 75.07 | 45.25 | 66.74 |
| WV-37095 | WV-40363 | 63.93 | 86.93 | 38.91 | 63.25 |
| WV-37096 | WV-40363 | 81.75 | 65.29 | 45.46 | 64.17 |
| WV-37097 | WV-40363 | 78.27 | 78.26 | 62.99 | 73.17 |
| WV-37098 | WV-40363 | 86.22 | 69.64 | 62.67 | 72.84 |
| WV-37099 | WV-40363 | 92.84 | 102.91 | 75.28 | 90.34 |
| WV-37100 | WV-40363 | 86.80 | 105.87 | 80.65 | 91.11 |
| WV-37101 | WV-40363 | 90.09 | N.D. | 80.37 | 85.23 |
| WV-37102 | WV-40363 | 110.46 | N.D. | 79.64 | 95.05 |
| WV-37103 | WV-40363 | 91.38 | N.D. | 62.20 | 76.79 |
| WV-37104 | WV-40363 | 106.24 | 97.43 | 74.67 | 92.78 |
| WV-37105 | WV-40363 | 94.52 | 118.40 | 87.02 | 99.98 |
| WV-37106 | WV-40363 | 110.23 | 106.37 | 87.68 | 101.43 |
| WV-37107 | WV-40363 | 73.41 | 63.23 | 51.23 | 62.62 |
| WV-37108 | WV-40363 | 53.31 | 61.67 | 48.05 | 54.35 |
| WV-37109 | WV-40363 | 71.54 | 69.66 | 37.27 | 59.49 |
| WV-37110 | WV-40363 | 70.73 | 42.09 | 29.78 | 47.54 |
| WV-37111 | WV-40363 | 70.38 | 67.85 | 40.31 | 59.51 |
| WV-37112 | WV-40363 | 52.80 | 35.91 | 35.50 | 41.40 |
| WV-37113 | WV-40363 | 54.65 | 51.00 | 47.96 | 51.20 |
| WV-37114 | WV-40363 | 64.66 | 55.32 | 53.88 | 57.95 |
| WV-37115 | WV-40363 | 80.22 | 85.29 | 70.83 | 78.78 |
| WV-37116 | WV-40363 | 82.32 | 73.21 | 60.83 | 72.12 |
| WV-37117 | WV-40363 | 91.16 | 106.76 | 59.72 | 85.88 |
| WV-37118 | WV-40363 | 88.95 | N.D. | 61.73 | 75.34 |
| WV-37119 | WV-40363 | 87.06 | 108.90 | 61.37 | 85.77 |
| WV-37120 | WV-40363 | 69.33 | 47.63 | 57.36 | 58.11 |
| WV-37121 | WV-40363 | 83.06 | 64.63 | 61.87 | 69.86 |
| WV-37122 | WV-40363 | 89.04 | 74.15 | 66.60 | 76.60 |
| WV-37123 | WV-40363 | 66.61 | 67.70 | 55.68 | 63.33 |
| WV-37124 | WV-40363 | 54.72 | 51.03 | 44.47 | 50.07 |
| WV-37125 | WV-40363 | 68.04 | 75.16 | 43.85 | 62.35 |
| WV-37126 | WV-40363 | 81.19 | 38.73 | 43.76 | 54.56 |
| WV-37127 | WV-40363 | 69.97 | 65.19 | 42.54 | 59.23 |
| WV-37128 | WV-40363 | 54.50 | 31.02 | 46.52 | 44.01 |
| WV-37129 | WV-40363 | 69.74 | 31.66 | 45.79 | 49.06 |
| WV-37130 | WV-40363 | 63.26 | 64.17 | 53.82 | 60.41 |
| WV-37131 | WV-40363 | 85.74 | 81.85 | 64.21 | 77.27 |
| WV-37132 | WV-40363 | 74.41 | 72.40 | 61.94 | 69.58 |
| WV-37133 | WV-40363 | 76.35 | 109.05 | 68.49 | 84.63 |
| WV-37134 | WV-40363 | 80.81 | 61.10 | 68.98 | 70.29 |
| WV-37135 | WV-40363 | 70.27 | 88.91 | 67.45 | 75.54 |
| WV-37136 | WV-40363 | 71.74 | 49.72 | 63.42 | 61.63 |
| WV-37137 | WV-40363 | 74.75 | 39.95 | 57.70 | 57.47 |
| WV-37138 | WV-40363 | 76.91 | 78.79 | 71.99 | 75.90 |
| WV-37139 | WV-40363 | 85.10 | 118.16 | 82.25 | 95.17 |
| WV-37140 | WV-40363 | 65.22 | 85.65 | 79.36 | 76.74 |
| WV-37141 | WV-40363 | 82.89 | 98.23 | 77.03 | 86.05 |
| WV-37142 | WV-40363 | 78.05 | 94.95 | 76.48 | 83.16 |
| WV-37143 | WV-40363 | 72.73 | 96.10 | 76.22 | 81.68 |
| WV-37144 | WV-40363 | 72.45 | 67.03 | 81.19 | 73.55 |
| WV-37145 | WV-40363 | 87.85 | 41.90 | 69.26 | 66.34 |
| WV-37146 | WV-40363 | 68.25 | 110.87 | 77.03 | 85.38 |
| WV-37147 | WV-40363 | 91.35 | 111.82 | 88.71 | 97.29 |
| WV-37148 | WV-40363 | 81.26 | 100.21 | 83.63 | 88.37 |
| WV-37149 | WV-40363 | 81.23 | N.D. | 92.34 | 86.79 |
| WV-37150 | WV-40363 | 98.26 | 104.62 | 94.14 | 99.01 |
| WV-37151 | WV-40363 | 82.19 | 92.28 | 92.33 | 88.93 |
| WV-20167 | WV-40362 | 25.41 | 9.93 | 18.86 | 18.07 |
| WV-37236 | WV-40362 | 47.17 | 44.06 | 48.95 | 46.73 |
| WV-20170 | WV-40363 | 23.31 | 19.94 | 14.62 | 19.29 |
| WV-20171 | WV-40363 | 19.22 | 12.22 | 13.90 | 15.11 |
| WV-37152 | WV-40363 | 81.43 | 116.00 | 99.34 | 98.92 |
| WV-37153 | WV-40363 | 66.15 | 108.96 | 82.97 | 86.03 |
| WV-37154 | WV-40363 | 70.77 | 94.58 | 82.26 | 82.54 |
| WV-37155 | WV-40363 | 57.31 | 92.50 | 73.90 | 74.57 |
| WV-37156 | WV-40363 | 69.22 | 81.84 | 72.13 | 74.40 |
| WV-37157 | WV-40363 | 57.31 | 83.63 | 63.59 | 68.17 |
| WV-37158 | WV-40363 | 68.76 | 88.30 | 78.61 | 78.56 |
| WV-37159 | WV-40363 | 55.45 | 70.25 | 67.00 | 64.23 |
| WV-37160 | WV-40363 | 72.72 | 105.03 | 72.62 | 83.46 |
| WV-37161 | WV-40363 | 63.65 | 95.48 | 95.81 | 84.98 |
| WV-37162 | WV-40363 | 71.57 | 90.01 | 77.88 | 79.82 |
| WV-37163 | WV-40363 | 62.89 | 109.96 | 81.41 | 84.75 |
| WV-37164 | WV-40363 | 66.01 | 97.50 | 73.34 | 78.95 |
| WV-37165 | WV-40363 | 67.84 | 115.09 | 64.75 | 82.56 |
| WV-37166 | WV-40363 | 69.37 | 102.39 | 75.43 | 82.39 |
| WV-37167 | WV-40363 | 65.23 | 98.14 | 94.50 | 85.96 |
| WV-37168 | WV-40363 | 86.02 | 118.98 | 89.14 | 98.05 |
| WV-37169 | WV-40363 | 69.59 | 107.27 | 94.57 | 90.48 |
| WV-37170 | WV-40363 | 79.65 | 97.59 | 86.05 | 87.76 |
| WV-37171 | WV-40363 | 54.13 | 86.04 | 67.82 | 69.33 |
| WV-37172 | WV-40363 | 64.78 | 83.85 | 62.75 | 70.46 |
| WV-37173 | WV-40363 | 58.37 | 87.53 | 56.38 | 67.43 |
| WV-37174 | WV-40363 | 52.31 | 78.40 | 50.54 | 60.42 |
| WV-37175 | WV-40363 | 54.99 | 72.36 | 55.89 | 61.08 |
| WV-37176 | WV-40363 | 68.93 | 86.39 | 60.23 | 71.85 |
| WV-37177 | WV-40363 | 61.36 | 75.04 | 63.30 | 66.57 |
| WV-37178 | WV-40363 | 62.13 | 70.14 | 64.90 | 65.72 |
| WV-37179 | WV-40363 | 65.59 | 108.12 | 74.72 | 82.81 |
| WV-37180 | WV-40363 | 67.31 | 97.38 | 63.01 | 75.90 |
| WV-37181 | WV-40363 | 72.43 | 119.58 | 74.07 | 88.69 |
| WV-37182 | WV-40363 | 65.68 | 103.40 | 66.21 | 78.43 |
| WV-37183 | WV-40363 | 68.63 | 101.30 | 81.83 | 83.92 |
| WV-37184 | WV-40363 | 78.97 | 99.09 | 74.35 | 84.14 |
| WV-37185 | WV-40363 | 63.16 | 90.15 | 80.39 | 77.90 |
| WV-37186 | WV-40363 | 73.32 | 96.56 | 80.73 | 83.54 |
| WV-37187 | WV-40363 | 68.28 | 92.44 | 69.12 | 76.62 |
| WV-37188 | WV-40363 | 62.68 | 82.97 | 54.37 | 66.67 |
| WV-37189 | WV-40363 | 66.04 | 93.30 | 57.50 | 72.28 |
| WV-37190 | WV-40363 | 62.97 | 85.61 | 55.21 | 67.93 |
| WV-37191 | WV-40363 | 68.69 | 76.09 | 67.45 | 70.75 |
| WV-37192 | WV-40363 | 76.93 | 101.08 | 73.64 | 83.89 |
| WV-37193 | WV-40363 | 63.65 | 85.98 | 80.02 | 76.55 |
| WV-37194 | WV-40363 | 64.99 | 79.58 | 69.55 | 71.37 |
| WV-37195 | WV-40363 | 68.17 | 106.57 | 89.41 | 88.05 |
| WV-37196 | WV-40363 | 81.13 | 106.89 | 70.99 | 86.34 |
| WV-37197 | WV-40363 | 79.71 | 93.62 | 81.27 | 84.87 |
| WV-37198 | WV-40363 | 81.35 | 64.66 | 83.68 | 76.56 |
| WV-37199 | WV-40363 | 72.53 | 82.00 | 84.40 | 79.64 |
| WV-37200 | WV-40363 | 77.80 | 87.97 | 94.87 | 86.88 |
| WV-37201 | WV-40363 | 75.95 | 99.62 | 97.56 | 91.04 |
| WV-37202 | WV-40363 | 85.71 | 101.63 | 85.58 | 90.98 |
| WV-37203 | WV-40363 | 80.48 | N.D. | 96.39 | 88.43 |
| WV-37204 | WV-40363 | 88.68 | N.D. | 84.78 | 86.73 |
| WV-37205 | WV-40363 | 83.50 | 123.63 | 90.60 | 99.24 |
| WV-37206 | WV-40363 | 79.70 | 76.56 | 90.94 | 82.40 |
| WV-37207 | WV-40363 | 81.40 | 90.27 | 95.63 | 89.10 |
| WV-37208 | WV-40363 | 79.81 | 100.45 | 101.89 | 94.05 |
| WV-37209 | WV-40363 | 79.32 | 107.44 | 98.19 | 94.98 |
| WV-37210 | WV-40363 | 82.40 | 110.28 | 90.04 | 94.24 |
| WV-37211 | WV-40363 | 85.82 | N.D. | 110.27 | 98.04 |
| WV-37212 | WV-40363 | 90.48 | N.D. | 92.64 | 91.56 |
| WV-37213 | WV-40363 | 96.27 | 108.92 | 99.78 | 101.66 |
| WV-37214 | WV-40363 | 88.00 | 70.45 | 94.26 | 84.23 |
| WV-37215 | WV-40363 | 80.98 | 62.33 | 104.43 | 82.58 |
| WV-37216 | WV-40363 | 79.70 | 113.05 | 92.37 | 95.04 |
| WV-37217 | WV-40363 | 78.95 | N.D. | 123.31 | 101.13 |
| WV-37218 | WV-40363 | 91.30 | N.D. | 106.43 | 98.87 |
| WV-37219 | WV-40363 | 84.48 | N.D. | 108.47 | 96.47 |
| WV-37220 | WV-40363 | 94.09 | N.D. | 95.69 | 94.89 |
| WV-37221 | WV-40363 | 88.67 | 100.47 | 103.07 | 97.41 |
| WV-37222 | WV-40363 | 88.56 | 72.58 | 92.17 | 84.44 |
| WV-37223 | WV-40363 | 90.04 | 65.69 | 102.97 | 86.23 |
| WV-37224 | WV-40363 | 82.10 | 102.78 | 86.94 | 90.61 |
| WV-37225 | WV-40363 | 80.35 | N.D. | 115.22 | 97.79 |
| WV-37226 | WV-40363 | 84.62 | 117.30 | 85.02 | 95.65 |
| WV-37227 | WV-40363 | 88.98 | N.D. | 109.16 | 99.07 |
| WV-37228 | WV-40363 | 100.11 | N.D. | 87.09 | 93.60 |
| WV-37229 | WV-40363 | 94.02 | 93.66 | 111.07 | 99.58 |
| WV-37230 | WV-40363 | 93.38 | 61.69 | 88.98 | 81.35 |
| WV-37231 | WV-40363 | 93.62 | 55.05 | 108.66 | 85.78 |
| WV-37232 | WV-40363 | 79.85 | 117.65 | 106.50 | 101.33 |
| WV-37233 | WV-40363 | 83.92 | N.D. | 101.53 | 92.72 |
| WV-37234 | WV-40363 | 82.58 | 120.43 | 106.39 | 103.14 |
| WV-37235 | WV-40363 | 48.12 | 67.78 | 46.97 | 54.29 |
| WV-20169 | WV-40363 | 31.37 | 26.20 | 19.12 | 25.56 |
| WV-20172 | WV-40363 | 20.11 | 7.80 | 10.24 | 12.72 |
| WV-49900 | WV-49901 | 95.37 | 101.03 | 108.67 | 101.69 |

Table 23 shows % mouse TTR mRNA remaining (at 500 and 1500 pM siRNA treatment) relative to mouse HPRT control. N=3. N.D.: Not determined.

TABLE 23

| | | 500 pM | | | | 1500 pM | | | |
|---|---|---|---|---|---|---|---|---|---|
| | | % remaining | % remaining | % remaining | | % remaining | % remaining | % remaining | |
| | | mRNA | mRNA | mRNA | | mRNA | mRNA | mRNA | |
| | | (mTTR/ | (mTTR/ | (mTTR/ | | (mTTR/ | (mTTR/ | (mTTR/ | |
| Guide | Passenger | mHPRT)-1 | mHPRT)-2 | mHPRT)-3 | Mean | mHPRT)-1 | mHPRT)-2 | mHPRT)-3 | Mean |
| WV-49900 | WV-49901 | 96.61 | 104.65 | 93.00 | 98.09 | 102.98 | 102.74 | 122.55 | 109.42 |
| WV-20167 | WV-40362 | 17.91 | 11.54 | 10.56 | 13.34 | 6.64 | 4.23 | 4.77 | 5.21 |
| WV-36836 | WV-40362 | 62.32 | 60.47 | 59.19 | 60.66 | 36.17 | 33.29 | 34.48 | 34.65 |
| WV-36837 | WV-40362 | 49.09 | 39.34 | 46.93 | 45.12 | 27.31 | 22.24 | 30.41 | 26.65 |
| WV-36838 | WV-40362 | 29.27 | 26.23 | 27.70 | 27.73 | 19.08 | 14.19 | 14.46 | 15.91 |
| WV-36839 | WV-40362 | 43.36 | 42.27 | 44.59 | 43.41 | 26.80 | 24.74 | 28.89 | 26.81 |
| WV-36840 | WV-40362 | 38.11 | 43.20 | 38.22 | 39.84 | 24.49 | 18.77 | 23.08 | 22.11 |
| WV-36841 | WV-40362 | 49.41 | 44.99 | 50.72 | 48.37 | 29.54 | 26.86 | 34.53 | 30.31 |
| WV-36842 | WV-40362 | 31.17 | 28.96 | 30.29 | 30.14 | 14.54 | 16.02 | 16.19 | 15.59 |
| WV-36843 | WV-40362 | 41.11 | 23.09 | 23.32 | 29.17 | 14.44 | 16.19 | 11.42 | 14.02 |
| WV-36844 | WV-40362 | 28.82 | 25.44 | 25.48 | 26.58 | 10.54 | 8.51 | 12.33 | 10.46 |
| WV-36845 | WV-40362 | 29.91 | 29.02 | 30.56 | 29.83 | 12.86 | 11.24 | 14.21 | 12.77 |
| WV-36846 | WV-40362 | 30.48 | 26.87 | 23.67 | 27.01 | 12.28 | 9.87 | 14.37 | 12.17 |
| WV-36847 | WV-40362 | 32.11 | 29.61 | 25.97 | 29.23 | 15.51 | 11.64 | 12.37 | 13.17 |
| WV-36848 | WV-40362 | 28.02 | 26.67 | 31.40 | 28.69 | 17.94 | 10.42 | 12.38 | 13.58 |
| WV-36849 | WV-40362 | 35.39 | 36.40 | 34.68 | 35.49 | 21.92 | 15.56 | 21.40 | 19.62 |
| WV-36850 | WV-40362 | 33.81 | 18.09 | 26.14 | 26.02 | 17.20 | 13.20 | 17.64 | 16.01 |
| WV-36851 | WV-40362 | 26.04 | 21.27 | 13.23 | 20.18 | 12.58 | 10.76 | 9.71 | 11.02 |
| WV-36852 | WV-40362 | 33.96 | 36.54 | 27.43 | 32.64 | 7.94 | 6.17 | 10.62 | 8.24 |
| WV-36853 | WV-40362 | 49.76 | 55.78 | 47.57 | 51.03 | 33.52 | 26.87 | 32.43 | 30.94 |
| WV-36854 | WV-40362 | 42.91 | 43.15 | 36.59 | 40.88 | 21.08 | 17.60 | 21.26 | 19.98 |
| WV-36855 | WV-40362 | 39.99 | 43.70 | 30.39 | 38.03 | 20.32 | 15.85 | 19.76 | 18.64 |
| WV-36856 | WV-40362 | 48.35 | 40.86 | 42.75 | 43.99 | 21.45 | 15.69 | 24.91 | 20.68 |
| WV-36857 | WV-40362 | 72.48 | 57.81 | 57.19 | 62.49 | 37.75 | 30.86 | 42.67 | 37.10 |

Table 24 shows % mouse TTR mRNA remaining (at 500 and 1500 pM siRNA treatment) relative to mouse HPRT control. N=3. N.D.: Not determined.

TABLE 24

| | | 500 pM | | | | 1500 pM | | | |
|---|---|---|---|---|---|---|---|---|---|
| | | % remaining | % remaining | % remaining | | % remaining | % remaining | % remaining | |
| | | mRNA | mRNA | mRNA | | mRNA | mRNA | mRNA | |
| | | (mTTR/ | (mTTR/ | (mTTR/ | | (mTTR/ | (mTTR/ | (mTTR/ | |
| Guide | Passenger | mHPRT)-1 | mHPRT)-2 | mHPRT)-3 | Mean | mHPRT)-1 | mHPRT)-2 | mHPRT)-3 | Mean |
| WV-49900 | WV-49901 | 90.55 | 92.26 | 96.49 | 93.10 | 103.35 | 118.79 | 93.06 | 105.07 |
| WV-20167 | WV-40362 | 25.76 | 32.74 | 34.81 | 31.10 | 8.76 | 11.52 | 14.18 | 11.49 |
| WV-36838 | WV-40362 | 32.55 | 38.48 | 37.46 | 36.16 | 12.87 | 14.69 | 13.14 | 13.57 |
| WV-36845 | WV-40362 | 33.49 | 46.85 | 43.41 | 41.25 | 16.15 | 14.34 | 16.54 | 15.68 |
| WV-36854 | WV-40362 | 29.72 | 34.37 | 35.94 | 33.34 | 10.86 | 12.20 | 19.33 | 14.13 |
| WV-38678 | WV-40362 | 31.40 | 44.34 | 43.96 | 39.90 | 15.55 | 13.16 | 14.27 | 14.33 |
| WV-38687 | WV-40362 | 31.21 | 33.60 | 32.79 | 32.54 | 13.05 | 13.44 | 13.27 | 13.25 |
| WV-20170 | WV-40363 | 17.99 | 22.48 | 20.63 | 20.37 | 8.05 | 5.52 | 6.20 | 6.59 |
| WV-38703 | WV-40363 | 36.78 | 37.33 | 31.04 | 35.05 | 16.11 | 13.47 | 14.33 | 14.64 |
| WV-38704 | WV-40363 | 48.19 | 56.91 | 49.79 | 51.63 | 26.87 | 25.35 | 27.10 | 26.44 |
| WV-41918 | WV-40363 | 24.29 | 26.83 | 24.11 | 25.08 | 5.22 | 6.23 | 8.77 | 6.74 |
| WV-41925 | WV-40363 | 25.41 | 31.13 | 28.58 | 28.37 | 9.99 | 10.35 | 10.66 | 10.33 |
| WV-41934 | WV-40363 | 25.74 | 28.13 | 25.86 | 26.57 | 7.94 | 10.08 | 15.55 | 11.19 |
| WV-38707 | WV-40363 | 22.04 | 24.31 | 24.16 | 23.50 | 6.30 | 5.87 | 7.44 | 6.54 |
| WV-38708 | WV-40363 | 24.85 | 27.89 | 26.78 | 26.51 | 6.42 | 7.74 | 9.47 | 7.88 |
| WV-40838 | WV-40363 | 38.51 | 41.06 | 42.70 | 40.76 | 15.26 | 12.39 | 9.95 | 12.53 |
| WV-40839 | WV-40363 | 50.47 | 48.58 | 52.39 | 50.48 | 33.57 | 37.89 | 26.20 | 32.55 |
| WV-40842 | WV-40363 | 53.10 | 48.90 | 53.20 | 51.73 | 19.46 | 25.07 | 21.93 | 22.15 |
| WV-40843 | WV-40363 | 65.04 | 61.53 | 59.49 | 62.02 | 32.06 | 36.87 | 36.26 | 35.06 |
| WV-41896 | WV-40363 | 96.44 | 92.60 | 95.23 | 94.76 | 73.85 | 77.28 | 75.46 | 75.53 |
| WV-41903 | WV-40363 | 29.14 | 25.01 | 20.86 | 25.00 | 9.03 | 9.60 | 14.22 | 10.95 |
| WV-41912 | WV-40363 | 29.67 | 27.07 | 28.10 | 28.28 | 9.20 | 11.53 | 9.97 | 10.24 |
| WV-38706 | WV-40363 | 79.23 | 86.07 | 79.89 | 81.73 | 66.47 | 63.93 | 78.87 | 69.76 |

Table 25 shows % mouse TTR mRNA remaining (at 150, 500, 1500, 5000, and 15000 pM siRNA treatment) relative to mouse HPRT control. N=3. N.D.: Not determined.

TABLE 25

| | | | % remaining | % remaining | % remaining | |
|---|---|---|---|---|---|---|
| | | | mRNA | mRNA | mRNA | |
| | | | (mTTR/ | (mTTR/ | (mTTR/ | |
| Guide | Passenger | Dosage | mHPRT)-1 | mHPRT)-2 | mHPRT)-3 | Mean |
| WV-49900 | WV-49901 | 150 pM | 96.02 | 102.77 | 102.38 | 100.39 |
| WV-20167 | WV-40362 | 150 pM | 50.24 | 45.69 | 40.70 | 45.54 |
| WV-20167 | WV-36860 | 150 pM | 60.23 | 44.49 | 49.48 | 51.40 |
| WV-20170 | WV-40363 | 150 pM | 29.35 | 31.39 | 38.84 | 33.19 |
| WV-41918 | WV-40363 | 150 pM | 30.58 | 23.71 | 25.83 | 26.71 |
| WV-38708 | WV-40363 | 150 pM | 41.80 | 36.11 | 42.92 | 40.28 |
| WV-41896 | WV-40363 | 150 pM | 76.48 | 74.42 | 81.44 | 77.44 |
| WV-38706 | WV-40363 | 150 pM | 69.26 | 87.69 | 95.06 | 84.00 |
| WV-20170 | WV-36807 | 150 pM | 40.23 | 37.81 | 41.33 | 39.79 |
| WV-41918 | WV-36807 | 150 pM | 38.50 | 36.74 | 42.94 | 39.40 |
| WV-38708 | WV-36807 | 150 pM | 49.77 | 47.00 | 45.74 | 47.51 |
| WV-41896 | WV-36807 | 150 pM | 110.74 | 104.22 | 105.25 | 106.73 |
| WV-38706 | WV-36807 | 150 pM | 88.06 | 95.42 | 101.64 | 95.04 |
| WV-49900 | WV-49901 | 500 pM | 104.23 | 98.91 | 94.84 | 99.32 |
| WV-20167 | WV-40362 | 500 pM | 26.60 | 24.42 | 24.60 | 25.21 |
| WV-20167 | WV-36860 | 500 pM | 33.06 | 30.03 | 30.31 | 31.13 |
| WV-20170 | WV-40363 | 500 pM | 25.84 | 16.47 | 16.80 | 19.71 |
| WV-41918 | WV-40363 | 500 pM | 17.64 | 13.55 | 15.87 | 15.69 |
| WV-38708 | WV-40363 | 500 pM | 26.42 | 24.94 | 28.92 | 26.76 |
| WV-41896 | WV-40363 | 500 pM | 87.12 | 85.93 | 78.95 | 84.00 |
| WV-38706 | WV-40363 | 500 pM | 81.56 | 74.33 | 71.97 | 75.95 |
| WV-20170 | WV-36807 | 500 pM | 26.76 | 22.18 | 23.85 | 24.26 |
| WV-41918 | WV-36807 | 500 pM | 24.84 | 21.85 | 22.05 | 22.91 |
| WV-38708 | WV-36807 | 500 pM | 29.50 | 31.09 | 28.94 | 29.84 |
| WV-41896 | WV-36807 | 500 pM | 103.68 | 143.26 | 143.16 | 130.03 |
| WV-38706 | WV-36807 | 500 pM | 107.55 | 100.97 | 88.24 | 98.92 |
| WV-49900 | WV-49901 | 1500 pM | 112.91 | 134.40 | 122.26 | 123.19 |
| WV-20167 | WV-40362 | 1500 pM | 8.19 | 7.67 | 8.29 | 8.05 |
| WV-20167 | WV-36860 | 1500 pM | 12.51 | 9.71 | 9.84 | 10.69 |
| WV-20170 | WV-40363 | 1500 pM | 5.16 | 4.74 | 4.53 | 4.81 |
| WV-41918 | WV-40363 | 1500 pM | 3.58 | 3.24 | 3.22 | 3.35 |
| WV-38708 | WV-40363 | 1500 pM | 8.26 | 5.16 | 6.62 | 6.68 |
| WV-41896 | WV-40363 | 1500 pM | 69.38 | 64.42 | 64.29 | 66.03 |
| WV-38706 | WV-40363 | 1500 pM | 51.02 | 55.38 | 54.11 | 53.50 |
| WV-20170 | WV-36807 | 1500 pM | 9.50 | 10.57 | 8.88 | 9.65 |
| WV-41918 | WV-36807 | 1500 pM | 7.08 | 5.12 | 6.06 | 6.09 |
| WV-38708 | WV-36807 | 1500 pM | 9.03 | 9.38 | 9.41 | 9.27 |
| WV-41896 | WV-36807 | 1500 pM | 98.23 | 95.36 | 99.42 | 97.67 |
| WV-38706 | WV-36807 | 1500 pM | 80.66 | 91.38 | 86.02 | 86.02 |
| WV-49900 | WV-49901 | 5000 pM | 100.07 | 102.27 | 81.87 | 94.73 |
| WV-20167 | WV-40362 | 5000 pM | 0.85 | 0.82 | 1.10 | 0.92 |
| WV-20167 | WV-36860 | 5000 pM | 0.99 | 1.57 | 1.37 | 1.31 |
| WV-20170 | WV-40363 | 5000 pM | 0.39 | 0.49 | 0.47 | 0.45 |
| WV-41918 | WV-40363 | 5000 pM | 0.29 | 0.25 | 0.29 | 0.28 |
| WV-38708 | WV-40363 | 5000 pM | 1.03 | 0.87 | 1.15 | 1.02 |
| WV-41896 | WV-40363 | 5000 pM | 27.21 | 32.14 | 31.79 | 30.38 |
| WV-38706 | WV-40363 | 5000 pM | 24.76 | 24.66 | 22.34 | 23.92 |
| WV-20170 | WV-36807 | 5000 pM | 0.94 | 0.80 | 0.71 | 0.81 |
| WV-41918 | WV-36807 | 5000 pM | 0.76 | 0.66 | 0.81 | 0.74 |
| WV-38708 | WV-36807 | 5000 pM | 1.62 | 1.33 | 1.50 | 1.48 |
| WV-41896 | WV-36807 | 5000 pM | 62.88 | 65.19 | 49.27 | 59.11 |
| WV-38706 | WV-36807 | 5000 pM | 48.98 | 47.68 | 43.73 | 46.80 |
| WV-49900 | WV-49901 | 15000 pM | 82.04 | 92.45 | 97.28 | 90.59 |
| WV-20167 | WV-40362 | 15000 pM | 0.13 | 0.11 | 0.11 | 0.12 |
| WV-20167 | WV-36860 | 15000 pM | 0.14 | 0.15 | 0.12 | 0.14 |
| WV-20170 | WV-40363 | 15000 pM | 0.07 | 0.07 | 0.08 | 0.08 |
| WV-41918 | WV-40363 | 15000 pM | 0.09 | 0.09 | 0.08 | 0.09 |
| WV-38708 | WV-40363 | 15000 pM | 0.10 | 0.09 | 0.13 | 0.11 |
| WV-41896 | WV-40363 | 15000 pM | 5.62 | 4.76 | 5.78 | 5.39 |
| WV-38706 | WV-40363 | 15000 pM | 2.85 | 3.86 | 3.30 | 3.34 |
| WV-20170 | WV-36807 | 15000 pM | 0.12 | 0.11 | 0.11 | 0.11 |
| WV-41918 | WV-36807 | 15000 pM | 0.12 | 0.10 | 0.10 | 0.11 |
| WV-38708 | WV-36807 | 15000 pM | 0.12 | 0.11 | 0.12 | 0.12 |
| WV-41896 | WV-36807 | 15000 pM | 15.33 | 16.07 | 19.18 | 16.86 |
| WV-38706 | WV-36807 | 15000 pM | 10.75 | 9.67 | 12.04 | 10.82 |

Table 26 shows % mouse TTR mRNA remaining (at 1500 pM siRNA treatment) relative to mouse HPRT control. N=3. N.D.: Not determined.

TABLE 26

| | | 1500 pM | | | |
|---|---|---|---|---|---|
| | | % remaining mRNA | % remaining mRNA | % remaining mRNA | |
| Guide | Passenger | (mTTR/mHPRT)-1 | (mTTR/mHPRT)-2 | (mTTR/mHPRT)-3 | Mean |
| WV-49900 | WV-49901 | 107.54 | 115.74 | 117.06 | 113.45 |
| WV-20167 | WV-40362 | 3.34 | 1.93 | 3.08 | 2.78 |
| WV-20167 | WV-36860 | 8.82 | 5.79 | 6.40 | 7.00 |
| WV-20170 | WV-40363 | 1.44 | 1.22 | 1.06 | 1.24 |
| WV-20171 | WV-40363 | 0.68 | 1.12 | 0.93 | 0.91 |
| WV-41896 | WV-40363 | 60.04 | 57.71 | 52.98 | 56.91 |
| WV-41918 | WV-40363 | 1.01 | 0.80 | 1.27 | 1.03 |
| WV-41940 | WV-40363 | 62.58 | 62.64 | 56.27 | 60.50 |
| WV-41962 | WV-40363 | 1.08 | 0.86 | 1.09 | 1.01 |
| WV-41898 | WV-40363 | 49.00 | 52.27 | 42.96 | 48.08 |
| WV-41920 | WV-40363 | 5.56 | 4.74 | 5.00 | 5.10 |
| WV-41942 | WV-40363 | 66.92 | 60.95 | 44.46 | 57.44 |
| WV-41964 | WV-40363 | 4.70 | 3.14 | 3.41 | 3.75 |
| WV-41903 | WV-40363 | 2.94 | 1.81 | 1.21 | 1.98 |
| WV-41925 | WV-40363 | 1.72 | 1.57 | 2.02 | 1.77 |
| WV-41947 | WV-40363 | 1.80 | 1.66 | 1.55 | 1.67 |
| WV-41969 | WV-40363 | 2.18 | 1.37 | 1.39 | 1.65 |
| WV-41912 | WV-40363 | 4.06 | 2.98 | 2.43 | 3.16 |
| WV-41934 | WV-40363 | 5.91 | 4.85 | 3.83 | 4.86 |
| WV-41956 | WV-40363 | 1.99 | 1.37 | 1.11 | 1.49 |
| WV-41978 | WV-40363 | 3.57 | 3.91 | 2.67 | 3.38 |
| WV-38707 | WV-40363 | 2.46 | 1.84 | 1.66 | 1.99 |
| WV-38705 | WV-40363 | 39.28 | 44.35 | 46.34 | 43.32 |
| WV-38708 | WV-40363 | 3.71 | 3.33 | 3.31 | 3.45 |
| WV-38706 | WV-40363 | 52.95 | 64.35 | 54.93 | 57.41 |
| WV-40838 | WV-40363 | 10.86 | 12.30 | 10.00 | 11.05 |
| WV-40839 | WV-40363 | 22.46 | 21.43 | 21.70 | 21.86 |
| WV-40842 | WV-40363 | 12.05 | 10.97 | 9.13 | 10.72 |
| WV-40843 | WV-40363 | 27.11 | 25.19 | 22.55 | 24.95 |
| WV-40552 | WV-40363 | 2.27 | 1.87 | 1.93 | 2.02 |
| WV-40796 | WV-40363 | 53.50 | 49.82 | 46.08 | 49.80 |
| WV-40553 | WV-40363 | 35.91 | 37.91 | 27.48 | 33.77 |
| WV-40797 | WV-40363 | 2.28 | 2.29 | 2.39 | 2.32 |
| WV-40555 | WV-40363 | 41.17 | 32.79 | 29.76 | 34.57 |
| WV-40556 | WV-40363 | 4.22 | 3.08 | 2.88 | 3.39 |
| WV-20170 | WV-36807 | 3.75 | 3.54 | 2.68 | 3.33 |
| WV-20171 | WV-36807 | 3.55 | 2.86 | 3.09 | 3.17 |
| WV-41896 | WV-36807 | 82.67 | 81.17 | 68.35 | 77.40 |
| WV-41918 | WV-36807 | 2.96 | 3.02 | 2.89 | 2.96 |
| WV-41940 | WV-36807 | 86.80 | 81.43 | 58.67 | 75.63 |
| WV-41962 | WV-36807 | 2.37 | 2.40 | 2.89 | 2.55 |
| WV-41898 | WV-36807 | 63.54 | 70.06 | 48.58 | 60.73 |
| WV-41920 | WV-36807 | 7.33 | 6.92 | 7.30 | 7.19 |
| WV-41942 | WV-36807 | 82.75 | 81.08 | 62.78 | 75.54 |
| WV-41964 | WV-36807 | 12.51 | 10.66 | 11.10 | 11.42 |
| WV-41903 | WV-36807 | 11.77 | 6.75 | 6.18 | 8.23 |
| WV-41925 | WV-36807 | 9.43 | 6.50 | 6.86 | 7.60 |
| WV-41947 | WV-36807 | 3.90 | 3.70 | 4.76 | 4.12 |
| WV-41969 | WV-36807 | 3.25 | 4.68 | 3.79 | 3.91 |
| WV-41912 | WV-36807 | 5.70 | 5.49 | 5.55 | 5.58 |
| WV-41934 | WV-36807 | 8.70 | 6.24 | 6.85 | 7.27 |
| WV-41956 | WV-36807 | 4.74 | 4.67 | 3.59 | 4.33 |
| WV-41978 | WV-36807 | 10.22 | 8.28 | 4.95 | 7.82 |
| WV-38707 | WV-36807 | 6.97 | 6.90 | 6.17 | 6.68 |
| WV-38705 | WV-36807 | 89.45 | 93.28 | 95.44 | 92.72 |
| WV-38708 | WV-36807 | 5.43 | 7.21 | 7.29 | 6.64 |
| WV-38706 | WV-36807 | 74.66 | 72.74 | 88.37 | 78.59 |
| WV-40838 | WV-36807 | 13.29 | 14.64 | 14.92 | 14.28 |
| WV-40839 | WV-36807 | 24.03 | 27.29 | 27.16 | 26.16 |
| WV-40842 | WV-36807 | 16.33 | 21.71 | 16.66 | 18.23 |
| WV-40843 | WV-36807 | 28.61 | 25.99 | 29.22 | 27.94 |
| WV-40552 | WV-36807 | 7.05 | 6.72 | 6.35 | 6.71 |
| WV-40796 | WV-36807 | 97.37 | 94.73 | 98.84 | 96.98 |
| WV-40553 | WV-36807 | 65.82 | 67.99 | 66.69 | 66.83 |
| WV-40797 | WV-36807 | 3.85 | 3.85 | 3.47 | 3.72 |
| WV-40555 | WV-36807 | 73.07 | 67.36 | 72.64 | 71.02 |
| WV-40556 | WV-36807 | 6.01 | 5.13 | 4.26 | 5.13 |

Table 27 shows % IC50 of knocking down mouse TTR mRNA in mouse primary hepatocyte

TABLE 27

| Guide | Passenger | IC50 (pM) | 95% CI |
|---|---|---|---|
| WV-20167 | WV-40362 | 229.6 | 164.6 to 320.7 |
| WV-43991 | WV-40363 | 230.4 | 166.7 to 319.8 |
| WV-43992 | WV-40363 | 233.4 | 126.6 to 436.7 |
| WV-43993 | WV-40363 | 132.4 | 90.62 to 194.4 |
| WV-43256 | WV-40363 | 157.7 | 97.46 to 108.5 |
| WV-43994 | WV-40363 | 150.8 | 106.3 to 214.1 |
| WV-41826 | WV-41828 | 158.7 | 113.8 to 221.6 |
| WV-42079 | WV-42080 | 89.14 | 98.86 to 112.9 |
| WV-43987 | WV-42080 | 115.7 | 60.19 to 219.5 |
| WV-43988 | WV-42080 | 77.39 | 53.21 to 113.0 |
| WV-43989 | WV-42080 | 176.9 | 95.86 to 326.1 |
| WV-43990 | WV-42080 | 181.4 | 96.94 to 345.4 |

Table 28 shows % IC50 of knocking down mouse TTR mRNA in mouse primary hepatocyte

TABLE 28

| Guide | Passenger | IC50 (pM) | 95% CI |
|---|---|---|---|
| WV-41826 | WV-41828 | 235.5 | 150.8 to 366.9 |
| WV-49611 | WV-41828 | 122.9 | 77.73 to 194.4 |
| WV-49612 | WV-41828 | 279.2 | 117.4 to 704.7 |
| WV-50481 | WV-41828 | 83.49 | 49.54 to 141.7 |
| WV-50482 | WV-41828 | 123 | 63.11 to 244.6 |
| WV-49626 | WV-42080 | 179.6 | 122.8 to 261.3 |
| WV-50485 | WV-42080 | 81.39 | 56.07 to 118.6 |
| WV-50486 | WV-42080 | 140.1 | 70.54 to 280.8 |
| WV-43775 | WV-42080 | 68.77 | 19.09 to 260.1 |
| WV-42079 | WV-42080 | 52.2 | 28.80 to 94.41 |
| WV-47145 | WV-42080 | 395.6 | 167.2 to 947.3 |
| WV-48528 | WV-42080 | 96.52 | 29.30 to 67.14 |
| WV-43988 | WV-42080 | 52.02 | 33.27 to 81.97 |
| WV-43989 | WV-42080 | 38.46 | 26.07 to 57.05 |

Table 29 shows % IC50 of knocking down mouse TTR mRNA in mouse primary hepatocyte

TABLE 29

| Guide | Passenger | IC50 (pM) | 95% CI |
|---|---|---|---|
| WV-49611 | WV-41828 | 99.01 | 78.70 to 124.6 |
| WV-49612 | WV-41828 | 202.5 | 118.4 to 343.8 |
| WV-51122 | WV-42080 | 60.81 | 43.14 to 85.75 |
| WV-47145 | WV-42080 | 80.05 | 67.43 to 94.97 |
| WV-48528 | WV-42080 | 78.69 | 50.64 to 122.2 |

Table 30 shows % mouse TTR mRNA remaining (at 50, 150 and 500 pM siRNA treatment) relative to mouse HPRT control. N=2. N.D.: Not determined.

TABLE 30

| | | 50 pM | | | 150 pM | | | 500 pM | | |
|---|---|---|---|---|---|---|---|---|---|---|
| | | % remaining | % remaining | | % remaining | % remaining | | % remaining | % remaining | |
| | | mRNA | mRNA | | mRNA | mRNA | | mRNA | mRNA | |
| | | (mTTR/ | (mTTR/ | | (mTTR/ | (mTTR/ | | (mTTR/ | (mTTR/ | |
| Guide | Passenger | mHPRT)-1 | mHPRT)-2 | Mean | mHPRT)-1 | mHPRT)-2 | Mean | mHPRT)-1 | mHPRT)-2 | Mean |
| WV- | WV- | 18.54 | 58.03 | 38.29 | 40.40 | 31.69 | 36.05 | 18.91 | 11.89 | 15.40 |
| 47145 | 42080 | | | | | | | | | |
| WV- | WV- | 66.14 | 66.80 | 66.47 | 52.11 | 34.67 | 43.39 | 25.05 | 12.72 | 18.88 |
| 48528 | 42080 | | | | | | | | | |
| WV- | WV- | 64.57 | 54.38 | 59.47 | 46.50 | 29.62 | 38.06 | 19.48 | 11.18 | 15.33 |
| 43775 | 42080 | | | | | | | | | |
| WV- | WV- | 17.77 | 61.43 | 39.60 | 42.33 | 37.07 | 39.70 | 10.11 | 15.08 | 12.60 |
| 50034 | 42080 | | | | | | | | | |
| WV- | WV- | 88.76 | 63.78 | 76.27 | 48.30 | 42.39 | 45.34 | 17.35 | 11.65 | 14.50 |
| 50035 | 42080 | | | | | | | | | |
| WV- | WV- | 83.38 | 61.35 | 72.36 | 54.55 | 32.65 | 43.60 | 16.24 | 17.73 | 16.99 |
| 50036 | 42080 | | | | | | | | | |
| WV- | WV- | 98.10 | 58.76 | 78.43 | 55.32 | 34.08 | 44.70 | 17.56 | 15.86 | 16.71 |
| 50037 | 42080 | | | | | | | | | |
| WV- | WV- | 84.62 | 71.78 | 78.20 | 59.95 | 46.31 | 53.13 | 19.75 | 18.18 | 18.96 |
| 50040 | 42080 | | | | | | | | | |
| WV- | WV- | 84.67 | 64.98 | 74.82 | 55.01 | 33.60 | 44.30 | 14.27 | 10.92 | 12.60 |
| 50041 | 42080 | | | | | | | | | |
| WV- | WV- | 96.57 | 56.59 | 76.58 | 42.37 | 37.27 | 39.82 | 15.71 | 9.42 | 12.57 |
| 50042 | 42080 | | | | | | | | | |
| WV- | WV- | 74.49 | 57.63 | 66.06 | 43.91 | 24.10 | 34.00 | 15.77 | 0.74 | 8.25 |
| 50043 | 42080 | | | | | | | | | |
| WV- | WV- | 80.75 | 75.71 | 78.23 | 42.51 | 36.57 | 39.54 | 17.82 | 11.97 | 14.90 |
| 50044 | 42080 | | | | | | | | | |
| WV- | WV- | 87.50 | 69.84 | 78.67 | 51.25 | 41.11 | 46.18 | 22.49 | 15.26 | 18.88 |
| 50045 | 42080 | | | | | | | | | |
| WV- | WV- | 83.16 | 59.91 | 71.54 | 49.10 | 32.75 | 40.92 | 16.77 | 10.74 | 13.76 |
| 50046 | 42080 | | | | | | | | | |
| WV- | WV- | 89.67 | 57.42 | 73.54 | 54.10 | 32.49 | 43.29 | 20.16 | 12.08 | 16.12 |
| 50047 | 42080 | | | | | | | | | |
| WV- | WV- | 67.39 | 67.33 | 67.36 | 39.72 | 36.22 | 37.97 | 20.67 | 13.05 | 16.86 |
| 50048 | 42080 | | | | | | | | | |
| WV- | WV- | 90.06 | 56.04 | 73.05 | 47.87 | 30.22 | 39.04 | 19.52 | 9.84 | 14.68 |
| 50049 | 42080 | | | | | | | | | |
| WV- | WV- | 80.29 | 64.17 | 72.23 | 56.55 | 46.21 | 51.38 | 26.75 | 18.92 | 22.83 |
| 50113 | 42080 | | | | | | | | | |
| WV- | WV- | 10.59 | 59.41 | 35.00 | 59.48 | 34.77 | 47.12 | 16.93 | 12.84 | 14.88 |
| 50114 | 42080 | | | | | | | | | |
| WV- | WV- | 83.46 | 73.69 | 78.57 | 61.56 | 50.80 | 56.18 | 27.45 | 22.91 | 25.18 |
| 50115 | 42080 | | | | | | | | | |
| WV- | WV- | 115.59 | 92.95 | 104.27 | 84.73 | 60.89 | 72.81 | 41.78 | 34.24 | 38.01 |
| 50116 | 42080 | | | | | | | | | |

Table 31 shows % mouse TTR mRNA remaining (at 50, 150 and 500 pM siRNA treatment) relative to mouse HPRT control. N=2. N.D. Not determined.

TABLE 31

| | | 50 pM | | | 150 pM | | | 500 pM | | |
|---|---|---|---|---|---|---|---|---|---|---|
| | | % remaining | % remaining | | % remaining | % remaining | | % remaining | % remaining | |
| | | mRNA | mRNA | | mRNA | mRNA | | mRNA | mRNA | |
| | | (mTTR/ | (mTTR/ | | (mTTR/ | (mTTR/ | | (mTTR/ | (mTTR/ | |
| Guide | Passenger | mHPRT)-1 | mHPRT)-2 | Mean | mHPRT)-1 | mHPRT)-2 | Mean | mHPRT)-1 | mHPRT)-2 | Mean |
| WV- | WV- | 66.90 | 52.48 | 59.69 | 40.32 | 34.62 | 37.47 | 10.54 | 12.78 | 11.66 |
| 47145 | 42080 | | | | | | | | | |
| WV- | WV- | 58.46 | 50.72 | 54.59 | 40.14 | 38.56 | 39.35 | 10.14 | 12.57 | 11.36 |
| 48528 | 42080 | | | | | | | | | |
| WV- | WV- | 54.15 | 54.27 | 54.21 | 44.02 | 38.81 | 41.41 | 11.45 | 14.96 | 13.21 |
| 50101 | 42080 | | | | | | | | | |
| WV- | WV- | 56.36 | 48.10 | 52.23 | 33.41 | 34.93 | 34.17 | 6.81 | 10.96 | 8.89 |
| 50102 | 42080 | | | | | | | | | |
| WV- | WV- | 79.14 | 75.75 | 77.44 | 51.09 | 61.68 | 56.38 | 16.49 | 23.00 | 19.74 |
| 50103 | 42080 | | | | | | | | | |
| WV- | WV- | 61.00 | 56.12 | 58.56 | 35.55 | 40.89 | 38.22 | 11.85 | 14.93 | 13.39 |
| 50104 | 42080 | | | | | | | | | |
| WV- | WV- | 62.71 | 56.09 | 59.40 | 46.62 | 37.68 | 42.15 | 10.24 | 15.09 | 12.66 |
| 50105 | 42080 | | | | | | | | | |
| WV- | WV- | 67.49 | 51.39 | 59.44 | 41.58 | 35.45 | 38.51 | 12.35 | 10.67 | 11.51 |
| 50106 | 42080 | | | | | | | | | |
| WV- | WV- | 65.67 | 52.18 | 58.92 | 35.21 | 39.04 | 37.12 | 11.30 | 10.41 | 10.86 |
| 50108 | 42080 | | | | | | | | | |
| WV- | WV- | 71.41 | 52.67 | 62.04 | 38.74 | 47.09 | 42.91 | 8.84 | 12.17 | 10.50 |
| 50110 | 42080 | | | | | | | | | |
| WV- | WV- | 60.47 | 52.99 | 56.73 | 37.03 | 35.37 | 36.20 | 9.84 | 10.07 | 9.95 |
| 50112 | 42080 | | | | | | | | | |

### Example 10. Provided Oligonucleotides and Compositions are Active In Vivo

In vivo determination of mouse TTR siRNA activity: All animal procedures were performed under IACUC guidelines. To evaluate the potency and liver exposure of provided oligonucleotides and compositions, male 8-10 weeks of age C₅₇BL/6 mice were dose at 2 mg/kg at desired oligonucleotide concentration on Day 1 by subcutaneous administration. Animals were euthanized on Day 8 by CO₂ asphyxiation followed by thoracotomy and terminal blood collection. After cardiac perfusion with PBS, liver samples were harvested and flash-frozen in dry ice. Liver total RNA was extracted using SV96 Total RNA Isolation kit (Promega), after tissue lysis with TRIzol and bromochloropropane. cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). For mouse TTR mRNA, the following qPCR assay were utilized: IDT Tagman qPCR assay ID Mm.PT.58.11922308. Oligonucleotide accumulation in liver was determined by hybrid ELISA.

Ago2 immunoprecipitation assay: Tissues (1 mpk dosed) were lysed in lysis buffer 50 mM Tris-HCl at pH 7.5, 200 mM NaCl, 0.5% Triton X-100, 2 mM EDTA, 1 mg/mL heparin) with protease inhibitor (Sigma-Aldrich). Lysate concentration was measured with a protein BCA kit (Pierce BCA protein assay kit or Bradford protein assay kit). Anti-Ago2 antibody was purchased from Wako Chemicals. Control mouse IgG was from eBioscience. Dynabeads (Invitrogen) were used to precipitate antibodies. Ago2-associated siRNA and endogenous miR122 were measured by Stem-Loop RT followed by TaqMan PCR analysis using Taqman miRNA and siRNA assay kit (Thermo fisher) based on manufacturer's methods.

To evaluate the durability of provided oligonucleotides and compositions, male 8-10 weeks of age C57BL/6 mice were dose at 6 mg/kg at desired oligonucleotide concentration on Day 1 by subcutaneous administration. On Day 1 (pre-dose) and then weekly, whole blood was collected via submandibular bleeding into serum separator tubes, and processed serum samples were kept at −70° C. Mouse TTR protein concentration in the serum was assessed using the Mouse Prealbumin ELISA kit (Crystal Chem) and following manufacturer's instructions.

Table 32 shows % mouse TTR protein remaining relative to PBS control. N=5. N.D.: Not determined.

TABLE 32

| | | WV-49900/WV- | | WV-20167/WV- | | WV-20169/WV- | |
|---|---|---|---|---|---|---|---|
| PBS | | 49901 | | 40362 | | 40363 | |
| | % remaining | | % remaining | | % remaining | | % remaining |
| animal | of mTTR | animal | of mTTR | animal | of mTTR | animal | of mTTR |
| No. | protein | No. | protein | No. | protein | No. | protein |
| 1 | 118 | 6 | 120 | 11 | 9 | 16 | 8 |
| 2 | 107 | 7 | 113 | 12 | 8 | 17 | 16 |
| 3 | 59 | 8 | 126 | 13 | 4 | 18 | 6 |
| 4 | 121 | 9 | 128 | 14 | 7 | 19 | 9 |
| 5 | 94 | 10 | 121 | 15 | 9 | 20 | 12 |
| Mean | 100 | Mean | 122 | Mean | 7 | Mean | 10 |
| WV-20170/WV- | | WV-20171/WV- | | WV-20172/WV- | | WV-20183/WV- | |
| 40363 | | 40363 | | 40363 | | 40363 | |
| | % remaining | | % remaining | | % remaining | | % remaining |
| animal | of mTTR | animal | of mTTR | animal | of mTTR | animal | of mTTR |
| No. | protein | No. | protein | No. | protein | No. | protein |
| 21 | 4 | 26 | 2 | 31 | 1 | 36 | 36 |
| 22 | 5 | 27 | 2 | 32 | 4 | 37 | 42 |
| 23 | 3 | 28 | 1 | 33 | 2 | 38 | 31 |
| 24 | 2 | 29 | 2 | 34 | 3 | 39 | 57 |
| 25 | 4 | 30 | 2 | 35 | 4 | 40 | 44 |
| Mean | 4 | Mean | 2 | Mean | 3 | Mean | 42 |

Table 33. shows the accumulation of antisense strand in liver tissue. N=5. N.D.: Not determined.

TABLE 33

| | | WV-49900/WV- | | WV-20167/WV- | | WV-20169/WV- | |
|---|---|---|---|---|---|---|---|
| PBS | | 49901 | | 40362 | | 40363 | |
| | antisense | | antisense | | antisense | | antisense |
| | strand | | strand | | strand | | strand |
| animal | (μg/g of | animal | (μg/g of | animal | (μg/g of | animal | (μg/g of |
| No. | tissue) | No. | tissue) | No. | tissue) | No. | tissue) |
| 1 | 0 | 6 | 0.684 | 11 | 0.309 | 16 | 0.568 |
| 2 | 0 | 7 | 0.588 | 12 | 0.255 | 17 | 0.733 |
| 3 | 0 | 8 | 0.527 | 13 | 0.653 | 18 | 0.599 |
| 4 | 0 | 9 | 0.517 | 14 | 0.388 | 19 | 0.470 |
| 5 | 0 | 10 | 0.547 | 15 | 0.540 | 20 | 0.250 |
| Mean | 0 | Mean | 0.573 | Mean | 0.429 | Mean | 0.524 |
| WV-20170/WV- | | WV-20171/WV- | | WV-20172/WV- | | WV-20183/WV- | |
| 40363 | | 40363 | | 40363 | | 40363 | |
| | antisense | | antisense | | antisense | | antisense |
| | strand | | strand | | strand | | strand |
| animal | (μg/g of | animal | (μg/g of | animal | (μg/g of | animal | (μg/g of |
| No. | tissue) | No. | tissue) | No. | tissue) | No. | tissue) |
| 21 | 0.676 | 26 | 0.526 | 31 | 0.753 | 36 | 0.034 |
| 22 | 0.671 | 27 | 1.352 | 32 | 0.771 | 37 | 0.032 |
| 23 | 0.798 | 28 | 1.038 | 33 | 0.880 | 38 | 0.023 |
| 24 | 0.685 | 29 | 0.570 | 34 | 0.820 | 39 | 0.041 |
| 25 | 0.707 | 30 | 1.156 | 35 | 0.900 | 40 | 0.045 |
| Mean | 0.707 | Mean | 0.929 | Mean | 0.825 | Mean | 0.035 |

Table 34 shows Ago 2 loading of guide strand relative to miR-122. N=5.

TABLE 34

| | | | | | Relative |
|---|---|---|---|---|---|
| | Ct: mTTR/Ago2 | Ct: miR-122/Ago2 | Ct: mTTR/IgG | Ct: miR-122/IgG | mTTR/miR122 |
| WV-20167/WV-40362-1 | 36.99 | 20.84 | 38.11 | 28.72 | 0.04 |
| WV-20167/WV-40362-2 | 32.95 | 20.68 | 37.78 | 28.61 | 1.16 |
| WV-20167/WV-40362-3 | 32.44 | 21.38 | 37.74 | 28.58 | 2.72 |
| WV-20167/WV-40362-4 | 31.97 | 20.51 | 37.73 | 28.48 | 2.08 |
| WV-20167/WV-40362-5 | 32.51 | 20.75 | 38.11 | 29.11 | 1.68 |
| WV-20169/WV-40363-1 | 32.81 | 22.55 | 38.27 | 29.45 | 4.75 |
| WV-20169/WV-40363-2 | 31.81 | 21.3 | 38.15 | 30.17 | 4.03 |
| WV-20169/WV-40363-3 | 36.63 | 22.06 | 38.57 | 30.52 | 0.18 |
| WV-20169/WV-40363-4 | 32.53 | 21.4 | 38.83 | 32.01 | 2.62 |
| WV-20169/WV-40363-5 | 36.71 | 21.99 | 38.68 | 31.27 | 0.16 |
| WV-20170/WV-40363-1 | 31.19 | 20.39 | 37.56 | 29.95 | 3.30 |
| WV-20170/WV-40363-2 | 30.54 | 20.17 | 37.51 | 29.97 | 4.46 |
| WV-20170/WV-40363-3 | 38.05 | 24.81 | 37.53 | 29.95 | −0.27 |
| WV-20170/WV-40363-4 | 30.67 | 20.08 | 37.5 | 30.39 | 3.83 |
| WV-20170/WV-40363-5 | 30.62 | 20.52 | 37.55 | 30.63 | 5.38 |
| WV-20171/WV-40363-1 | 31.62 | 22.04 | 37.87 | 31.14 | 7.68 |
| WV-20171/WV-40363-2 | 36.56 | 22.62 | 38.11 | 31.25 | 0.25 |
| WV-20171/WV-40363-3 | 30.75 | 21.16 | 38.33 | 31.52 | 7.69 |
| WV-20171/WV-40363-4 | 32.62 | 22.61 | 38.27 | 32.34 | 5.66 |
| WV-20171/WV-40363-5 | 36.86 | 23.58 | 38.61 | 32.52 | 0.42 |
| WV-20172/WV-40363-1 | 32.2 | 20.94 | 38.03 | 29.18 | 2.39 |
| WV-20172/WV-40363-2 | 32.82 | 22.25 | 37.67 | 28.15 | 3.78 |
| WV-20172/WV-40363-3 | 31.19 | 21.11 | 37.74 | 28.52 | 5.44 |
| WV-20172/WV-40363-4 | 31.57 | 20.4 | 37.96 | 29.1 | 2.55 |
| WV-20172/WV-40363-5 | 31.65 | 20.84 | 37.84 | 29.14 | 3.27 |
| WV-20183/WV-40363-1 | 37.62 | 20.1 | 38.11 | 29.18 | 0.01 |
| WV-20183/WV-40363-2 | 38.09 | 21.49 | 38.08 | 29.06 | 0.00 |
| WV-20183/WV-40363-3 | 38.24 | 22.65 | 38.04 | 29.28 | −0.02 |
| WV-20183/WV-40363-4 | 38.4 | 23.17 | 38.12 | 28.22 | −0.03 |
| WV-20183/WV-40363-5 | 38.53 | 24.65 | 38.5 | 29.43 | −0.01 |

Table 35. shows % mouse TTR protein remaining relative to PBS control. N=5. N.D.: Not determined.

TABLE 35

| | PBS | | | | | |
|---|---|---|---|---|---|---|
| Day | animal1 | animal2 | animal3 | animal4 | animal5 | Mean |
| 1 | 105 | 102 | 89 | 102 | 103 | 100 |
| 8 | 109 | 81 | 103 | 99 | 109 | 100 |
| 15 | 94 | 111 | 92 | 100 | 103 | 100 |
| 22 | 82 | 115 | 110 | 79 | 113 | 100 |
| 29 | 117 | 93 | 91 | 98 | 101 | 100 |
| 36 | 122 | 79 | 94 | 96 | 109 | 100 |
| 43 | 123 | 97 | 109 | 82 | 89 | 100 |
| 50 | 112 | 105 | 87 | 97 | 98 | 100 |
| 64 | 104 | 98 | 95 | 94 | 108 | 100 |
| | WV-20167/WV-40362 | | | | | |
| Day | animal6 | animal7 | animal8 | animal9 | animal10 | Mean |
| 1 | 121 | 102 | 103 | N.D. | 106 | 108 |
| 8 | 2 | 2 | 2 | N.D. | 2 | 2 |
| 15 | 2 | 2 | 3 | N.D. | 2 | 2 |
| 22 | 3 | 5 | 3 | N.D. | 2 | 3 |
| 29 | 5 | 16 | 10 | N.D. | 7 | 10 |
| 36 | 21 | 34 | 21 | N.D. | 15 | 23 |
| 43 | 41 | 56 | 40 | N.D. | 35 | 43 |
| 50 | 76 | 107 | 91 | N.D. | 68 | 86 |
| 64 | 77 | 98 | 114 | N.D. | 85 | 93 |
| | WV-49900/WV-49901 | | | | | |
| Day | animal11 | animal12 | animal13 | animal14 | animal15 | Mean |
| 1 | 109 | 121 | 79 | 114 | 136 | 112 |
| 8 | 100 | 109 | 77 | 108 | 107 | 100 |
| 15 | 103 | 92 | 79 | 111 | 92 | 96 |
| 22 | 82 | 86 | 86 | 78 | 142 | 95 |
| 29 | 110 | 99 | 87 | 95 | 113 | 101 |
| 36 | 96 | 87 | 83 | 107 | 93 | 93 |
| 43 | 91 | 99 | 49 | 79 | 88 | 81 |
| 50 | 106 | 112 | 80 | 100 | 144 | 108 |
| 64 | 101 | 103 | 83 | 99 | 120 | 101 |
| | WV-20169/WV-40363 | | | | | |
| Day | animal16 | animal17 | animal18 | animal19 | animal20 | Mean |
| 1 | 78 | 126 | 111 | 96 | 124 | 107 |
| 8 | 2 | 2 | 2 | 3 | 2 | 2 |
| 15 | 3 | 2 | 2 | 8 | 8 | 5 |
| 22 | 12 | 4 | 5 | 30 | 6 | 11 |
| 29 | 49 | 21 | 24 | 69 | 29 | 38 |
| 36 | 64 | 40 | 49 | 68 | 42 | 53 |
| 43 | 72 | 60 | 72 | 87 | 89 | 76 |
| 50 | N.D. | N.D. | N.D. | N.D. | N.D. | N.D. |
| 64 | N.D. | N.D. | N.D. | N.D. | N.D. | N.D. |
| | WV-20170/WV-40363 | | | | | |
| Day | animal21 | animal22 | animal23 | animal24 | animal25 | Mean |
| 1 | 132 | 110 | 74 | 106 | 85 | 102 |
| 8 | 2 | 1 | 2 | 2 | 2 | 2 |
| 15 | 2 | 2 | 1 | 2 | 2 | 2 |
| 22 | 2 | 1 | 1 | 1 | 1 | 1 |
| 29 | 2 | 2 | 2 | 2 | 2 | 2 |
| 36 | 2 | 5 | 4 | 3 | 3 | 3 |
| 43 | 4 | 10 | 10 | 6 | 6 | 7 |
| 50 | 18 | 28 | 33 | 19 | 20 | 23 |
| 64 | 60 | 80 | 68 | 62 | 56 | 65 |
| | WV-20171/WV-40363 | | | | | |
| Day | animal26 | animal27 | animal28 | animal29 | animal30 | Mean |
| 1 | 91 | 76 | 82 | 73 | 96 | 83 |
| 8 | 1 | 1 | 1 | 1 | 2 | 1 |
| 15 | 1 | 1 | 1 | 1 | 1 | 1 |
| 22 | 1 | 1 | 1 | 2 | 1 | 1 |
| 29 | 5 | 5 | 3 | 11 | 3 | 5 |
| 36 | 14 | 14 | 9 | 20 | 12 | 14 |
| 43 | 32 | 39 | 21 | 40 | 27 | 32 |
| 50 | 69 | 75 | 59 | 77 | 74 | 71 |
| 64 | 97 | 94 | 79 | 75 | 89 | 87 |
| | WV-20172/WV-40363 | | | | | |
| Day | animal31 | animal32 | animal33 | animal34 | animal35 | Mean |
| 1 | 97 | 78 | 97 | 87 | 103 | 92 |
| 8 | 2 | 2 | 2 | 2 | 2 | 2 |
| 15 | 1 | 1 | 1 | 1 | 2 | 1 |
| 22 | 1 | 1 | 1 | 1 | 1 | 1 |
| 29 | 4 | 3 | 2 | 2 | 2 | 3 |
| 36 | 9 | 6 | 5 | 4 | 4 | 6 |
| 43 | 27 | 14 | 13 | 7 | 11 | 14 |
| 50 | 57 | 35 | 36 | 28 | 34 | 38 |
| 64 | 90 | 94 | 81 | 87 | 76 | 86 |
| | WV-20183/WV-40363 | | | | | |
| Day | animal36 | animal37 | animal38 | animal39 | animal40 | Mean |
| 1 | 91 | 81 | 117 | 106 | 86 | 96 |
| 8 | 7 | 6 | 7 | 6 | 6 | 6 |
| 15 | 16 | 13 | 19 | 14 | 17 | 16 |
| 22 | 25 | 24 | 30 | 25 | 23 | 26 |
| 29 | 81 | 79 | 84 | 76 | 80 | 80 |
| 36 | 83 | 89 | 84 | 79 | 83 | 84 |
| 43 | N.D. | N.D. | N.D. | N.D. | N.D. | N.D. |
| 50 | N.D. | N.D. | N.D. | N.D. | N.D. | N.D. |
| 64 | N.D. | N.D. | N.D. | N.D. | N.D. | N.D. |

### Example 11. Provided Oligonucleotides and Compositions are Active In Vivo

In vivo determination of mouse TTR siRNA activity: All animal procedures were performed under IACUC guidelines. To evaluate the durability of provided oligonucleotides and compositions, male 8-10 weeks of age C57B3L/6 mice were dose at 2 mg/kg at desired oligonucleotide concentration on Day 1 by subcutaneous administration. On Day 1 (pre-dose) and then weekly, whole blood was collected via submandibular bleeding into serum separator tubes, and processed serum samples were kept at −70° C. Mouse TTR protein concentration in the serum was assessed using the Mouse Prealbumin ELISA kit (Crystal Chem) and following manufacturer's instructions.

Table 36. shows % mouse TTR protein remaining relative to PBS control. N=5. N.D.: Not determined.

TABLE 36

| | PBS | | | | | |
|---|---|---|---|---|---|---|
| Day | animal1 | animal2 | animal3 | animal4 | animal5 | Mean |
| 1 | 99 | 95 | 98 | 98 | 110 | 100 |
| 8 | 98 | 103 | 121 | 89 | 88 | 100 |
| 15 | 83 | 100 | 115 | 88 | 113 | 100 |
| 22 | 78 | 104 | 126 | 96 | 96 | 100 |
| 29 | 96 | 104 | 113 | 97 | 90 | 100 |
| 36 | 102 | 95 | 118 | 83 | 102 | 100 |
| 43 | 98 | 82 | 116 | 86 | 118 | 100 |
| 50 | 94 | 92 | 112 | 99 | 103 | 100 |
| | WV-49900/WV-49901 | | | | | |
| Day | animal6 | animal7 | animal8 | animal9 | animal10 | Mean |
| 1 | 116 | 89 | 92 | 71 | 108 | 95 |
| 8 | 120 | 80 | 74 | 75 | 113 | 92 |
| 15 | 112 | 97 | 102 | 87 | 108 | 101 |
| 22 | 134 | 108 | 109 | 107 | 117 | 115 |
| 29 | 113 | 91 | 100 | 108 | 123 | 107 |
| 36 | 113 | 86 | 84 | 76 | 113 | 94 |
| 43 | 119 | 97 | 127 | 117 | 113 | 115 |
| 50 | 119 | 90 | 89 | 83 | 111 | 99 |
| | WV-20167/WV-40362 | | | | | |
| Day | animal11 | animal12 | animal13 | animal14 | animal15 | Mean |
| 1 | 100 | 79 | 97 | 96 | 91 | 93 |
| 8 | 6 | 6 | 3 | 4 | 4 | 5 |
| 15 | 11 | 16 | 5 | 8 | 11 | 10 |
| 22 | 30 | 59 | 18 | 24 | 25 | 31 |
| 29 | 67 | 85 | 39 | 68 | 64 | 65 |
| 36 | 79 | 78 | 54 | 74 | 72 | 71 |
| 43 | 113 | 69 | 104 | 108 | 111 | 101 |
| 50 | 106 | 93 | 97 | 95 | 95 | 97 |
| | WV-20171/WV-40363 | | | | | |
| Day | animal16 | animal17 | animal18 | animal19 | animal20 | Mean |
| 1 | 92 | 103 | 66 | 86 | 102 | 90 |
| 8 | 3 | 2 | 4 | 3 | 2 | 3 |
| 15 | 4 | 5 | 9 | 4 | 5 | 5 |
| 22 | 7 | 15 | 29 | 14 | 18 | 16 |
| 29 | 29 | 54 | 70 | 49 | 45 | 49 |
| 36 | 59 | 94 | 76 | 87 | 77 | 79 |
| 43 | 112 | 112 | 99 | 110 | 102 | 107 |
| 50 | 99 | 109 | 93 | 110 | 94 | 101 |
| | WV-43256/WV-40363 | | | | | |
| Day | animal21 | animal22 | animal23 | animal24 | animal25 | Mean |
| 1 | 90 | 84 | 92 | 93 | 99 | 92 |
| 8 | 3 | 4 | 4 | 4 | 4 | 4 |
| 15 | 5 | 5 | 5 | 4 | 7 | 5 |
| 22 | 13 | 13 | 13 | 14 | 12 | 13 |
| 29 | 37 | 35 | 32 | 35 | 36 | 35 |
| 36 | 64 | 64 | 56 | 60 | 63 | 61 |
| 43 | 88 | 96 | 95 | 101 | 103 | 97 |
| 50 | 101 | 101 | 97 | 99 | 104 | 100 |
| | WV-20170/WV-40363 | | | | | |
| Day | animal26 | animal27 | animal28 | animal29 | animal30 | Mean |
| 1 | 81 | 61 | 62 | 84 | 101 | 78 |
| 8 | 4 | 4 | 6 | 3 | 3 | 4 |
| 15 | 3 | 9 | 9 | 5 | 3 | 6 |
| 22 | 7 | 16 | 19 | 11 | 6 | 12 |
| 29 | 20 | 40 | 40 | 24 | 16 | 28 |
| 36 | 37 | 48 | 56 | 34 | 35 | 42 |
| 43 | 69 | 86 | 70 | 79 | 127 | 86 |
| 50 | 87 | 82 | 67 | 94 | 84 | 83 |
| | WV-38708/WV-40363 | | | | | |
| Day | animal31 | animal32 | animal33 | animal34 | animal35 | Mean |
| 1 | 87 | 98 | 75 | 53 | 77 | 78 |
| 8 | 3 | 3 | 4 | 5 | 3 | 3 |
| 15 | 5 | 4 | 5 | 8 | 3 | 5 |
| 22 | 11 | 4 | 8 | 16 | 9 | 10 |
| 29 | 27 | 12 | 22 | 43 | 27 | 26 |
| 36 | 52 | 22 | 41 | 50 | 47 | 42 |
| 43 | 91 | 44 | 61 | 78 | 91 | 73 |
| 50 | 87 | 62 | 91 | 71 | 89 | 80 |
| | WV-38708/WV-36807 | | | | | |
| Day | animal36 | animal37 | animal38 | animal39 | animal40 | Mean |
| 1 | 83 | 81 | 87 | 106 | 104 | 92 |
| 8 | N.D. | 16 | 13 | 21 | 11 | 15 |
| 15 | 27 | 32 | 29 | 36 | 23 | 29 |
| 22 | 52 | 53 | 47 | 58 | 56 | 53 |
| 29 | 84 | 89 | 98 | 92 | 77 | 88 |
| 36 | 94 | 81 | 96 | 89 | 83 | 88 |
| 43 | 101 | 102 | 113 | 115 | 99 | 106 |
| 50 | 96 | 103 | 99 | 99 | 91 | 97 |

### Example 12. Provided Oligonucleotides and Compositions are Active In Vivo

In vivo determination of mouse TTR siRNA activity: All animal procedures were performed under IACUC guidelines. To evaluate the potency and liver exposure of provided oligonucleotides and compositions, male 8-10 weeks of age C57BL/6 mice were dose at 0.5 mg/kg at desired oligonucleotide concentration on Day 1 by subcutaneous administration. Animals were euthanized on Day 8 by CO₂ asphyxiation followed by thoracotomy and terminal blood collection. After cardiac perfusion with PBS, liver samples were harvested and flash-frozen in dry ice. Liver total RNA was extracted using SV96 Total RNA Isolation kit (Promega), after tissue lysis with TRIzol and bromochloropropane. cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). For mouse TTR mRNA, the following qPCR assay were utilized: IDT Taqman qPCR assay ID Mm.PT.58.11922308. Oligonucleotide accumulation in liver was determined by hybrid ELISA.

Ago2 immunoprecipitation assay: Tissues (1 mpk dosed) were lysed in lysis buffer 50 mM Tris-HCl at pH 7.5, 200 mM NaCl, 0.5% Triton X-100, 2 mM EDTA, 1 mg/mL heparin) with protease inhibitor (Sigma-Aldrich). Lysate concentration was measured with a protein BCA kit (Pierce BCA protein assay kit or Bradford protein assay kit). Anti-Ago2 antibody was purchased from Wako Chemicals. Control mouse IgG was from eBioscience. Dynabeads (Invitrogen) were used to precipitate antibodies. Ago2-associated siRNA and endogenous miR122 were measured by Stem-Loop RT followed by TaqMan PCR analysis using Taqman miRNA and siRNA assay kit (Thermo fisher) based on manufacturer's methods.

To evaluate the durability of provided oligonucleotides and compositions, male 8-10 weeks of age C57BL/6 mice were dose at 0.5 mg/kg at desired oligonucleotide concentration on Day 1 by subcutaneous administration. On Day 1 (pre-dose) and then weekly, whole blood was collected via submandibular bleeding into serum separator tubes, and processed serum samples were kept at −70° C. Mouse TTR protein concentration in the serum was assessed using the Mouse Prealbumin ELISA kit (Crystal Chem) and following manufacturer's instructions.

Table 37 shows % mouse TTR protein remaining relative to PBS control. N=5. N.D.: Not determined.

TABLE 37

| | | WV-49613/WV- | | WV-49614/WV- | | WV-49611/WV- | |
|---|---|---|---|---|---|---|---|
| PBS | | 49615 | | 49615 | | 41828 | |
| | % remaining | | % remaining | | % remaining | | % remaining |
| animal | of mTTR | animal | of mTTR | animal | of mTTR | animal | of mTTR |
| No. | protein | No. | protein | No. | protein | No. | protein |
| 1 | 69 | 6 | 85 | 11 | 96 | 16 | 14 |
| 2 | 66 | 7 | 94 | 12 | 103 | 17 | 10 |
| 3 | 147 | 8 | 112 | 13 | 62 | 18 | 20 |
| 4 | 103 | 9 | 91 | 14 | 109 | 19 | 18 |
| 5 | 116 | 10 | 100 | 15 | 84 | 20 | 23 |
| Mean | 100 | Mean | 96 | Mean | 91 | Mean | 17 |
| WV-49612/WV- | | WV-51122/WV- | | WV-47145/WV- | | WV-48528/WV- | |
| 41828 | | 42080 | | 42080 | | 42080 | |
| | % remaining | | % remaining | | % remaining | | % remaining |
| animal | of mTTR | animal | of mTTR | animal | of mTTR | animal | of mTTR |
| No. | protein | No. | protein | No. | protein | No. | protein |
| 21 | 25 | 26 | 8 | 31 | 14 | 36 | 8 |
| 22 | 15 | 27 | 16 | 32 | 4 | 37 | 6 |
| 23 | 38 | 28 | 8 | 33 | 7 | 38 | 5 |
| 24 | 11 | 29 | 5 | 34 | 5 | 39 | 7 |
| 25 | 18 | 30 | 12 | 35 | 9 | 40 | 9 |
| Mean | 22 | Mean | 10 | Mean | 8 | Mean | 7 |

Table 38. shows the accumulation of antisense strand in liver tissue. N=5. N.D.: Not determined.

TABLE 38

| | | WV-49613/WV- | | WV-49614/WV- | | WV-49611/WV- | |
|---|---|---|---|---|---|---|---|
| PBS | | 49615 | | 49615 | | 41828 | |
| | antisense | | antisense | | antisense | | antisense |
| | strand | | strand | | strand | | strand |
| animal | (μg/g of | animal | (μg/g of | animal | (μg/g of | animal | (μg/g of |
| No. | tissue) | No. | tissue) | No. | tissue) | No. | tissue) |
| 1 | 0 | 6 | 0.059 | 11 | 0.098 | 16 | 0.015 |
| 2 | 0 | 7 | 0.066 | 12 | 0.109 | 17 | 0.018 |
| 3 | 0 | 8 | 0.069 | 13 | 0.103 | 18 | 0.023 |
| 4 | 0 | 9 | 0.060 | 14 | 0.107 | 19 | 0.019 |
| 5 | 0 | 10 | 0.063 | 15 | 0.108 | 20 | 0.024 |
| Mean | 0 | Mean | 0.063 | Mean | 0.105 | Mean | 0.020 |
| WV-49612/WV- | | WV-51122/WV- | | WV-47145/WV- | | WV-48528/WV- | |
| 41828 | | 42080 | | 42080 | | 42080 | |
| | antisense | | antisense | | antisense | | antisense |
| | strand | | strand | | strand | | strand |
| animal | (μg/g of | animal | (μg/g of | animal | (μg/g of | animal | (μg/g of |
| No. | tissue) | No. | tissue) | No. | tissue) | No. | tissue) |
| 21 | 0.038 | 26 | 0.023 | 31 | 0.053 | 36 | 0.077 |
| 22 | 0.047 | 27 | 0.017 | 32 | 0.065 | 37 | 0.071 |
| 23 | 0.041 | 28 | 0.044 | 33 | 0.073 | 38 | 0.060 |
| 24 | 0.014 | 29 | 0.050 | 34 | 0.057 | 39 | 0.061 |
| 25 | 0.063 | 30 | 0.044 | 35 | 0.066 | 40 | 0.062 |
| Mean | 0.040 | Mean | 0.036 | Mean | 0.063 | Mean | 0.066 |

Table 39 shows Ago 2 loading of guide strand relative to miR-122. N=5.

TABLE 39

| | | | | | Relative |
|---|---|---|---|---|---|
| | Ct: mTTR/Ago2 | Ct: miR-122/Ago2 | Ct: mTTR/IgG | Ct: miR-122/IgG | mTTR/miR122 |
| WV-49611/WV-41828-1 | 39.03 | 21.41 | 39.3 | 32.01 | 0.18 |
| WV-49611/WV-41828-2 | 38.49 | 20.14 | 39.37 | 30.37 | 0.28 |
| WV-49611/WV-41828-3 | 38.15 | 20.46 | 39.35 | 31.72 | 0.55 |
| WV-49611/WV-41828-4 | 38.35 | 20.2 | 39.4 | 30.51 | 0.37 |
| WV-49611/WV-41828-5 | 38.64 | 22.15 | 39.36 | 29.69 | 0.88 |
| WV-49612/WV-41828-1 | 38.35 | 20.91 | 39.57 | 29.59 | 0.66 |
| WV-49612/WV-41828-2 | 38.92 | 21.82 | 39.73 | 29.81 | 0.63 |
| WV-49612/WV-41828-3 | 38.73 | 20.9 | 39.26 | 30.62 | 0.27 |
| WV-49612/WV-41828-4 | 37.57 | 20.78 | 39.68 | 30.29 | 1.40 |
| WV-49612/WV-41828-5 | 38.63 | 21.32 | 39.65 | 32.23 | 0.65 |
| WV-51122/WV-42080-1 | 39.43 | 23.27 | 39.39 | 30.26 | −0.08 |
| WV-51122/WV-42080-2 | 38.69 | 21.45 | 39.52 | 30.24 | 0.59 |
| WV-51122/WV-42080-3 | 38.11 | 21.58 | 39.49 | 30.93 | 1.35 |
| WV-51122/WV-42080-4 | 37.48 | 21.79 | 39.35 | 30.74 | 2.85 |
| WV-51122/WV-42080-5 | 37.67 | 21.54 | 39.23 | 30.61 | 1.91 |
| WV-47145/WV-42080-1 | 37.54 | 21.76 | 39.43 | 30.89 | 2.69 |
| WV-47145/WV-42080-2 | 37.19 | 21.63 | 39.19 | 31.43 | 3.22 |
| WV-47145/WV-42080-3 | 37.67 | 21.18 | 39.53 | 31.39 | 1.63 |
| WV-47145/WV-42080-4 | 37.54 | 21.68 | 39.23 | 32.22 | 2.40 |
| WV-47145/WV-42080-5 | 38.04 | 22.62 | 39.22 | 30.66 | 2.64 |
| WV-48528/WV-40363-1 | 37.47 | 21.51 | 39.68 | 30.15 | 2.55 |
| WV-48528/WV-40363-2 | 37.14 | 20.67 | 39.05 | 29.81 | 1.67 |
| WV-48528/WV-40363-3 | 37.15 | 20.74 | 39.1 | 30.03 | 1.76 |
| WV-48528/WV-40363-4 | 37.35 | 21.03 | 39.35 | 29.6 | 1.90 |
| WV-48528/WV-40363-5 | 37.1 | 20.69 | 39.55 | 30.47 | 1.94 |

Table 40. shows % mouse TTR protein remaining relative to PBS control. N=5. N.D.: Not determined.

TABLE 40

| | PBS | | | | | |
|---|---|---|---|---|---|---|
| Day | animal1 | animal2 | animal3 | animal4 | animal5 | Mean |
| 1 | 105 | 85 | 100 | 101 | 109 | 100 |
| 8 | 115 | 89 | 100 | 95 | 101 | 100 |
| 15 | 110 | 91 | 97 | 98 | 103 | 100 |
| 29 | 114 | 100 | 91 | 89 | 106 | 100 |
| 43 | 143 | 80 | 87 | 85 | 105 | 100 |
| | WV-49613/WV-49615 | | | | | |
| Day | animal6 | animal7 | animal 8 | animal9 | animal10 | Mean |
| 1 | 71 | 94 | 104 | 129 | 99 | 99 |
| 8 | 73 | 81 | 89 | 79 | 76 | 79 |
| 15 | 93 | 90 | 91 | 112 | 103 | 98 |
| 29 | 81 | 76 | 84 | 86 | 79 | 81 |
| 43 | 77 | 79 | 99 | N.D. | 98 | 88 |
| | WV-49614/WV-49615 | | | | | |
| Day | animal11 | animal12 | animal13 | animal14 | animal15 | Mean |
| 1 | 119 | 83 | 110 | 105 | 107 | 105 |
| 8 | 62 | N.D. | 72 | 60 | 73 | 67 |
| 15 | 79 | 78 | 105 | 90 | 100 | 90 |
| 29 | 78 | 64 | 89 | 82 | 86 | 80 |
| 43 | 106 | 68 | 103 | 116 | 106 | 100 |
| | WV-49611/WV-41828 | | | | | |
| Day | animal16 | animal17 | animal18 | animal19 | animal20 | Mean |
| 1 | 110 | 100 | 120 | 114 | 116 | 112 |
| 8 | 16 | 19 | 22 | 18 | 26 | 21 |
| 15 | 26 | 34 | 29 | 24 | 42 | 31 |
| 29 | 39 | 49 | 65 | 54 | 65 | 54 |
| 43 | 80 | 99 | 76 | 82 | 76 | 83 |
| | WV-49612/WV-41828 | | | | | |
| Day | animal21 | animal22 | animal23 | animal24 | animal25 | Mean |
| 1 | 114 | 117 | 103 | 122 | 134 | 118 |
| 8 | 66 | 66 | 84 | 50 | 48 | 63 |
| 15 | 46 | 52 | 79 | 54 | 35 | 53 |
| 29 | 70 | 53 | 66 | 54 | 47 | 58 |
| 43 | 87 | 119 | 88 | 100 | 90 | 97 |
| | WV-51122/WV-42080 | | | | | |
| Day | animal26 | animal27 | animal28 | animal29 | animal30 | Mean |
| 1 | 143 | 120 | 126 | 115 | 118 | 124 |
| 8 | 13 | 13 | 8 | 15 | 12 | 12 |
| 15 | 15 | 33 | 14 | 25 | 15 | 20 |
| 22 | 52 | N.D. | 44 | 69 | 39 | 51 |
| 29 | 86 | 83 | 84 | 96 | 99 | 90 |
| | WV-47145/WV-42080 | | | | | |
| Day | animal31 | animal32 | animal33 | animal34 | animal35 | Mean |
| 1 | 111 | 121 | 114 | 116 | 95 | 111 |
| 8 | 9 | 6 | 11 | 16 | 11 | 11 |
| 15 | 13 | 13 | 9 | 17 | 17 | 14 |
| 29 | 42 | 32 | 30 | 37 | 43 | 37 |
| 43 | 71 | 69 | 77 | 69 | 80 | 73 |
| | WV-48528/WV-42080 | | | | | |
| Day | animal36 | animal37 | animal38 | animal39 | animal40 | Mean |
| 1 | 90 | 83 | 98 | 94 | 95 | 92 |
| 8 | 10 | 16 | 7 | 5 | 10 | 9 |
| 15 | 11 | 21 | 17 | 9 | 10 | 14 |
| 29 | 28 | 40 | 42 | 28 | 24 | 32 |
| 43 | 61 | 88 | 120 | 69 | 61 | 80 |

### Example 13. Provided Oligonucleotides and Compositions are Active In Vivo

In vivo determination of mouse TTR siRNA activity: All animal procedures were performed under IACUC guidelines. To evaluate the potency and liver exposure of provided oligonucleotides and compositions, male 8-10 weeks of age C57BL/6 mice were dose at 2 mg/kg at desired oligonucleotide concentration on Day 1 by subcutaneous administration. Animals were euthanized on Day 8 by CO₂ asphyxiation followed by thoracotomy and terminal blood collection. After cardiac perfusion with PBS, liver samples were harvested and flash-frozen in dry ice. Liver total RNA was extracted using SV96 Total RNA Isolation kit (Promega), after tissue lysis with TRIzol and bromochloropropane. cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). For mouse TTR mRNA, the following qPCR assay were utilized: IDT Taqman qPCR assay ID Mm.PT.58.11922308. Oligonucleotide accumulation in liver was determined by hybrid ELISA.

Ago2 immunoprecipitation assay: Tissues (1 mpk dosed) were lysed in lysis buffer 50 mM Tris-HCl at pH 7.5, 200 mM NaCl, 0.5% Triton X-100, 2 mM EDTA, 1 mg/mL heparin) with protease inhibitor (Sigma-Aldrich). Lysate concentration was measured with a protein BCA kit (Pierce BCA protein assay kit or Bradford protein assay kit). Anti-Ago2 antibody was purchased from Wako Chemicals. Control mouse IgG was from eBioscience. Dynabeads (Invitrogen) were used to precipitate antibodies. Ago2-associated siRNA and endogenous miR122 were measured by Stem-Loop RT followed by TaqMan PCR analysis using Taqman miRNA and siRNA assay kit (Thermo fisher) based on manufacturer's methods.

Table 41 shows % mouse TTR protein remaining relative to PBS control. N=5. ND.: Not determined.

TABLE 41

| | | WV-49900/WV- | | WV-20167/WV- | | WV-20170/WV- | |
|---|---|---|---|---|---|---|---|
| PBS | | 49901 | | 40362 | | 40363 | |
| | % remaining | | % remaining | | % remaining | | % remaining |
| animal | of mTTR | animal | of mTTR | animal | of mTTR | animal | of mTTR |
| No. | protein | No. | protein | No. | protein | No. | protein |
| 1 | 112 | 6 | 111 | 11 | 9 | 16 | 6 |
| 2 | 122 | 7 | 100 | 12 | 11 | 17 | 8 |
| 3 | 85 | 8 | 112 | 13 | 9 | 18 | 8 |
| 4 | 74 | 9 | 113 | 14 | 5 | 19 | 6 |
| 5 | 106 | 10 | 116 | 15 | 6 | 20 | 5 |
| Mean | 100 | Mean | 110 | Mean | 8 | Mean | 7 |
| WV-41918/WV- | | WV-41896/WV- | | WV-38708/WV- | | WV-38706/WV- | |
| 40363 | | 40363 | | 40363 | | 40363 | |
| | % remaining | | % remaining | | % remaining | | % remaining |
| animal | of mTTR | animal | of mTTR | animal | of mTTR | animal | of mTTR |
| No. | protein | No. | protein | No. | protein | No. | protein |
| 21 | 1 | 26 | 117 | 31 | 6 | 36 | 120 |
| 22 | 3 | 27 | 99 | 32 | 6 | 37 | 117 |
| 23 | 2 | 28 | 96 | 33 | 7 | 38 | 124 |
| 24 | 1 | 29 | 110 | 34 | 8 | 39 | 132 |
| 25 | 4 | 30 | 105 | 35 | 6 | 40 | 111 |
| Mean | 2 | Mean | 105 | Mean | 6 | Mean | 121 |

Table 42. shows the accumulation of antisense strand in liver tissue. N=5. N.D.: Not determined.

TABLE 42

| | | WV-49900/WV- | | WV-20167/WV- | | WV-20170/WV- | |
|---|---|---|---|---|---|---|---|
| PBS | | 49901 | | 40362 | | 40363 | |
| | antisense | | antisense | | antisense | | antisense |
| | strand | | strand | | strand | | strand |
| animal | (μg/g of | animal | (μg/g of | animal | (μg/g of | animal | (μg/g of |
| No. | tissue) | No. | tissue) | No. | tissue) | No. | tissue) |
| 1 | 0 | 6 | 0.073 | 11 | 0.051 | 16 | 0.053 |
| 2 | 0 | 7 | 0.072 | 12 | 0.064 | 17 | 0.152 |
| 3 | 0 | 8 | 0.085 | 13 | 0.070 | 18 | 0.078 |
| 4 | 0 | 9 | 0.087 | 14 | 0.076 | 19 | 0.043 |
| 5 | 0 | 10 | 0.087 | 15 | 0.110 | 20 | 0.105 |
| Mean | 0 | Mean | 0.081 | Mean | 0.074 | Mean | 0.086 |
| WV-41918/WV- | | WV-41896/WV- | | WV-38708/WV- | | WV-38706/WV- | |
| 40363 | | 40363 | | 40363 | | 40363 | |
| | antisense | | antisense | | antisense | | antisense |
| | strand | | strand | | strand | | strand |
| animal | (μg/g of | animal | (μg/g of | animal | (μg/g of | animal | (μg/g of |
| No. | tissue) | No. | tissue) | No. | tissue) | No. | tissue) |
| 21 | 0.337 | 26 | 0.148 | 31 | 0.195 | 36 | 0.080 |
| 22 | 0.242 | 27 | 0.059 | 32 | 0.149 | 37 | 0.164 |
| 23 | 0.205 | 28 | 0.134 | 33 | 0.257 | 38 | 0.053 |
| 24 | 0.181 | 29 | 0.165 | 34 | 0.144 | 39 | 0.049 |
| 25 | 0.145 | 30 | 0.120 | 35 | 0.259 | 40 | 0.030 |
| Mean | 0.222 | Mean | 0.125 | Mean | 0.201 | Mean | 0.075 |

Table 43 shows Ago 2 loading of guide strand relative to miR-122. N=5.

TABLE 43

| | | | | | Relative |
|---|---|---|---|---|---|
| | Ct: mTTR/Ago2 | Ct: miR-122/Ago2 | Ct: mTTR/IgG | Ct: miR-122/IgG | mTTR/miR122 |
| WV-20167/WV-40362-1 | 28.82 | 17.83 | 34.06 | 29.89 | 0.80 |
| WV-20167/WV-40362-2 | 28.9 | 18.14 | 33.62 | 29.13 | 0.93 |
| WV-20167/WV-40362-3 | 28.88 | 17.27 | 32.91 | 28.41 | 0.50 |
| WV-20167/WV-40362-4 | 27.79 | 17.45 | 32.57 | 29.09 | 1.25 |
| WV-20167/WV-40362-5 | 28.03 | 18.17 | 32 | 24.73 | 1.69 |
| WV-20170/WV-40363-1 | 29.36 | 20.25 | 32.49 | 30.44 | 2.69 |
| WV-20170/WV-40363-2 | 28.76 | 18.36 | 34.63 | 30.96 | 1.22 |
| WV-20170/WV-40363-3 | 30.14 | 18.21 | 33.17 | 31.1 | 0.38 |
| WV-20170/WV-40363-4 | 29.38 | 19.09 | 32.85 | 30.55 | 1.22 |
| WV-20170/WV-40363-5 | 28.91 | 18.37 | 32.48 | 30.09 | 1.03 |
| WV-41918/WV-40363-1 | 29.03 | 18.41 | 31.51 | 29.47 | 0.87 |
| WV-41918/WV-40363-2 | 28.7 | 18.17 | 31.76 | 29.68 | 1.00 |
| WV-41918/WV-40363-3 | 29.1 | 19.86 | 32 | 29.84 | 2.40 |
| WV-41918/WV-40363-4 | 28.36 | 18.2 | 31.11 | 29.57 | 1.25 |
| WV-41918/WV-40363-5 | 28.45 | 18.46 | 31.4 | 30.18 | 1.44 |
| WV-41896/WV-40363-1 | 26.69 | 18.14 | 31.81 | 29.42 | 4.35 |
| WV-41896/WV-40363-2 | 26.74 | 18.31 | 33.12 | 30.13 | 4.81 |
| WV-41896/WV-40363-3 | 27 | 18.11 | 31.98 | 30.09 | 3.42 |
| WV-41896/WV-40363-4 | 27.18 | 17.38 | 31.65 | 29.45 | 1.80 |
| WV-41896/WV-40363-5 | 26.17 | 18.1 | 32.28 | 28.84 | 6.15 |
| WV-38708/WV-40363-1 | 29.84 | 17.74 | 32.16 | 28.71 | 0.31 |
| WV-38708/WV-40363-2 | 30.07 | 17.6 | 32.23 | 29.24 | 0.23 |
| WV-38708/WV-40363-3 | 30.75 | 17.42 | 31.54 | 29.35 | 0.07 |
| WV-38708/WV-40363-4 | 30.1 | 17.94 | 31.59 | 30.05 | 0.24 |
| WV-38708/WV-40363-5 | 30.61 | 19.05 | 31.2 | 30.81 | 0.19 |
| WV-38706/WV-40363-1 | 28.36 | 19.26 | 31.79 | 29.26 | 2.77 |
| WV-38706/WV-40363-2 | 28.07 | 18.96 | 30.91 | 28.21 | 2.61 |
| WV-38706/WV-40363-3 | 27.6 | 19.48 | 31.88 | 28.47 | 5.72 |
| WV-38706/WV-40363-4 | 28.26 | 18.68 | 31.65 | 28.19 | 1.98 |
| WV-38706/WV-40363-5 | 28.21 | 18.34 | 31.91 | 28.65 | 1.65 |

### Example 14. In Vitro Off-Target Analysis of Provided Oligonucleotides and Compositions by RNAseq

Various siRNAs for mouse TTR were designed and constructed. In order to evaluate the off-target effects of stereochemistry, a number of siRNAs were tested in vitro in mouse primary hepatocytes. siRNAs were gymnotically delivered to mouse primary hepatocytes plated at 24-well plates, with 40,000 cells/well. Final siRNA concentration is either 0.2 or 2 μM. Following 48 hours treatment, total RNA was extracted using SV96 Total RNA Isolation kit (Promega). cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). Library was prepared using QuantSeq 3′-mRNA-Seq library preparation kit (Lexogen GmbH) following manufacturer's protocol. Sequencing was performed on NovaSeq SP chip at Harvard Core Facility. Off-target effects were evaluated by using DEseq2 to determine the differentially expressed genes compared with sample with PBS treatment.

Table 44 shows gene numbers for downregulated, unchanged, and upregulateded genes.

TABLE 44

| | 0.2 μM siRNA | | | 2 μM siRNA | | |
|---|---|---|---|---|---|---|
| | Downregulated | Unchanged | Upregulated | Downregulated | Unchanged | Upregulated |
| WV- | 0 | 55421 | 0 | 1 | 55420 | 0 |
| 49613/WV- | | | | | | |
| 49615 | | | | | | |
| WV- | 0 | 55421 | 0 | 2 | 55419 | 0 |
| 49614/WV- | | | | | | |
| 49615 | | | | | | |
| WV- | 3 | 55418 | 0 | 9 | 55403 | 9 |
| 41826/WV- | | | | | | |
| 41828 | | | | | | |
| WV- | 9 | 55409 | 3 | 48 | 55370 | 3 |
| 49611/WV- | | | | | | |
| 41828 | | | | | | |
| WV- | 1 | 55419 | 1 | 8 | 55411 | 2 |
| 49612/WV- | | | | | | |
| 41828 | | | | | | |
| WV- | 1 | 55420 | 0 | 127 | 55288 | 6 |
| 49626/WV- | | | | | | |
| 42080 | | | | | | |
| WV- | 4 | 55415 | 2 | 7 | 55413 | 1 |
| 43775/WV- | | | | | | |
| 42080 | | | | | | |
| WV- | 3 | 55416 | 2 | 96 | 55312 | 13 |
| 51122/WV- | | | | | | |
| 42080 | | | | | | |
| WV- | 2 | 55418 | 1 | 9 | 55411 | 1 |
| 47145/WV- | | | | | | |
| 42080 | | | | | | |
| WV- | 5 | 55413 | 3 | 6 | 55411 | 4 |
| 48528/WV- | | | | | | |
| 42080 | | | | | | |

### Example 15. Provided Oligonucleotides and Compositions are Well Tolerated in Wild Type Mice

All animal procedures were performed under IACUC guidelines. To evaluate the impacts of provided oligonucleotides and compositions on liver function, male 8-10 weeks of age C57BL/6 mice were dosed at 1.5 or 15 mg/kg at desired oligonucleotide concentration on Day 1, 8, and 15, by subcutaneous administration. Animals were euthanized on Day 16 by CO₂ asphyxiation followed by thoracotomy and terminal blood collection. Terminal serum were analyzed at Charles River Laboratories (CRL Shrewsbury, MA) using clinically validated assays on AU640 instrument.

Table 45 shows serum biomarker results after repeated dosage in wild type mice. N=5. N.D.: Not determined. ALT, alanine transaminase; AST, aspartate transaminase; ALP, alkaline phosphatase; ALB, albumin; TP, total protein.

TABLE 45

| | | | | | |
|---|---|---|---|---|---|
| Animal | ALT | AST | ALP | ALB | TP |
| No. | (U/L) | (U/L) | (U/L) | (g/dL) | (g/dL) |

PBS

| | | | | | |
|---|---|---|---|---|---|
| 1 | 29 | 54 | 52 | 3.8 | 6.1 |
| 2 | 23 | 43 | 47 | 3.7 | 5.5 |
| 3 | 22 | 54 | 53 | 3.8 | 6 |
| 4 | 24 | 116 | 73 | 4.3 | 6.7 |
| 5 | 27 | 59 | 97 | 3.8 | 6.2 |
| Mean | 25 | 65 | 64 | 3.9 | 6.1 |

WV-49611/WV-41828, 1.5 mg/kg

| | | | | | |
|---|---|---|---|---|---|
| 6 | 30 | 67 | 47 | 3.4 | 5.7 |
| 7 | N.D. | N.D. | N.D. | N.D. | 6.1 |
| 8 | 22 | 51 | 52 | 3.8 | 5.9 |
| 9 | 34 | 89 | 68 | 3.7 | 5.8 |
| 10 | 25 | 84 | 86 | 4.1 | 5.9 |
| Mean | 28 | 73 | 63 | 3.8 | 5.9 |

WV-49611/WV-41828, 15 mg/kg

| | | | | | |
|---|---|---|---|---|---|
| 11 | 63 | 188 | 77 | 4 | 6.2 |
| 12 | 25 | 39 | 66 | 4 | 6.3 |
| 13 | 35 | 76 | 52 | 3.7 | 5.6 |
| 14 | 27 | 40 | 61 | 3.6 | 6 |
| 15 | 25 | 69 | 65 | 4.1 | 6.1 |
| Mean | 35 | 82 | 64 | 3.9 | 6 |

WV-51122/WV-42080, 1.5 mg/kg

| | | | | | |
|---|---|---|---|---|---|
| 16 | 32 | 45 | 47 | 3.5 | 5.9 |
| 17 | 216 | 182 | 67 | 3.8 | 6.2 |
| 18 | 18 | 67 | 45 | 3.6 | 5.7 |
| 19 | 28 | 79 | 73 | 3.9 | 6.3 |
| 20 | 49 | 72 | 75 | 4 | 6.1 |
| Mean | 69 | 89 | 61 | 3.8 | 6 |

WV-51122/WV-42080, 15 mg/kg

| | | | | | |
|---|---|---|---|---|---|
| 21 | 26 | 61 | 68 | 3.8 | 6.1 |
| 22 | 34 | 77 | 75 | 3.7 | 6.2 |
| 23 | 87 | 86 | 77 | 3.7 | 5.6 |
| 24 | 27 | 57 | 84 | 4.1 | 6.2 |
| 25 | 25 | 70 | 49 | 4.2 | 6.6 |
| Mean | 40 | 70 | 71 | 3.9 | 6.1 |

WV-47145/WV-42080, 1.5 mg/kg

| | | | | | |
|---|---|---|---|---|---|
| 26 | 25 | 51 | 51 | 3.6 | 6 |
| 27 | 25 | 46 | 57 | 3.5 | 5.8 |
| 28 | 24 | 43 | 62 | 4 | 6.5 |
| 29 | 45 | 61 | 82 | 3.8 | 6 |
| 30 | 53 | 145 | 83 | 4 | 6.4 |
| Mean | 34 | 69 | 67 | 3.8 | 6.1 |

WV-47145/WV-42080, 15 mg/kg

| | | | | | |
|---|---|---|---|---|---|
| 31 | 27 | 94 | 71 | 3.7 | 5.9 |
| 32 | 19 | 48 | 72 | 3.8 | 6.1 |
| 33 | N.D | N.D | N.D | N.D | N.D |
| 34 | 44 | 157 | 61 | 4.3 | 6.8 |
| 35 | 41 | 90 | 119 | 4.1 | 6.3 |
| Mean | 33 | 97 | 81 | 4 | 6.3 |

WV-48528/WV-42080, 1.5 mg/kg

| | | | | | |
|---|---|---|---|---|---|
| 36 | 22 | 34 | 71 | 3.5 | 5.7 |
| 37 | 37 | 55 | 130 | 3.4 | 5.7 |
| 38 | 31 | 153 | 46 | 3.1 | 6.6 |
| 39 | 23 | 52 | 52 | 3.8 | 5.9 |
| 40 | 25 | 74 | 66 | 3.8 | 6 |
| Mean | 28 | 74 | 73 | 3.5 | 6 |

WV-48528/WV-42080, 15 mg/kg

| | | | | | |
|---|---|---|---|---|---|
| 41 | 31 | 41 | 59 | 4.2 | 6.9 |
| 42 | 26 | 48 | 62 | 4 | 6.6 |
| 43 | 32 | 47 | 66 | 3.3 | 5.5 |
| 44 | 45 | 55 | 72 | 3.5 | 6 |
| 45 | 29 | 53 | 96 | 4 | 6.2 |
| Mean | 33 | 49 | 71 | 3.8 | 6.2 |

### Example 16. Provided Oligonucleotides and Compositions can Effectively Knockdown Mouse Transthyretin (mTTR) In Vitro

Various siRNAs for mouse TTR were designed and constructed. A number of siRNAs were tested in vitro in mouse primary hepatocytes at one or a range of concentrations. Some siRNAs were also tested in mice (e.g., C57BL6 wild type mice).

Example protocol for in vitro determination of siRNA activity: For determination of siRNAs activity, siRNAs at specific concentration were gymnotically delivered to mouse primary hepatocytes plated at 96-well plates, with 10,000 cells/well. Following 48 hours treatment, total RNA was extracted using SV96 Total RNA Isolation kit (Promega). cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). For mouse TTR mRNA, the following qPCR assay were utilized: IDT Tagman qPCR assay ID Mm.PT.58.11922308. Mouse HPRT was used as normalizer (Forward 5′CAAACTTTGCTTTCCCTGGTT3′, Reverse 5′TGGCCTGTATCCAACACTTC3′, Probe 5′/5HEX/ACCAGCAAG/Zen/CTTGCAACCTTAACC/3IABkFQ/3′. mRNA knockdown levels were calculated as % mRNA remaining relative to mock treatment.

Table 46 shows % mouse TTR mRNA remaining (at 300 and 100 pM siRNA treatment) relative to mouse HPRT control. N=2. N.D.: Not determined.

TABLE 46

| | | 300 pM | | | 100 pM | | |
|---|---|---|---|---|---|---|---|
| | | % remaining | % remaining | | % remaining | % remaining | |
| | | mRNA | mRNA | | mRNA | mRNA | |
| | | (mTTR/ | (mTTR/ | | (mTTR/ | (mTTR/ | |
| Guide | Passenger | mHPRT)-1 | mHPRT)-2 | Mean | mHPRT)-1 | mHPRT)-2 | Mean |
| SSR- | SSR- | 40.58 | 39.32 | 39.95 | 77.48 | 73.72 | 75.60 |
| 0106266 | 0101599 | | | | | | |
| SSR- | SSR- | 61.23 | 50.89 | 56.06 | 61.81 | 78.26 | 70.03 |
| 0106267 | 0101599 | | | | | | |
| SSR- | SSR- | 55.89 | 43.04 | 49.47 | 66.19 | 70.50 | 68.35 |
| 0106268 | 0101599 | | | | | | |
| SSR- | SSR- | 60.70 | 53.03 | 56.86 | 74.07 | 81.77 | 77.92 |
| 0106269 | 0101599 | | | | | | |
| SSR- | SSR- | 60.63 | 45.75 | 53.19 | 64.28 | 81.32 | 72.80 |
| 0106270 | 0101599 | | | | | | |
| SSR- | SSR- | 62.29 | 51.39 | 56.84 | 70.23 | 84.40 | 77.31 |
| 0106271 | 0101599 | | | | | | |
| SSR- | SSR- | 62.17 | 45.95 | 54.06 | 69.93 | 69.11 | 69.52 |
| 0106272 | 0101599 | | | | | | |
| SSR- | SSR- | 51.62 | 43.00 | 47.31 | 67.08 | 75.29 | 71.18 |
| 0106273 | 0101599 | | | | | | |
| SSR- | SSR- | 51.90 | 51.53 | 51.71 | 77.55 | 77.38 | 77.46 |
| 0106274 | 0101599 | | | | | | |
| SSR- | SSR- | 58.87 | 59.18 | 59.03 | 81.56 | 77.43 | 79.50 |
| 0106275 | 0101599 | | | | | | |
| SSR- | SSR- | 59.69 | 57.31 | 58.50 | 85.75 | 71.66 | 78.70 |
| 0106276 | 0101599 | | | | | | |
| SSR- | SSR- | 70.71 | 66.60 | 68.65 | 95.92 | 87.92 | 91.92 |
| 0106277 | 0101599 | | | | | | |
| SSR- | SSR- | 78.51 | 66.29 | 72.40 | 81.03 | 84.23 | 82.63 |
| 0106278 | 0101599 | | | | | | |
| SSR- | SSR- | 81.88 | 79.31 | 80.59 | 96.76 | 100.94 | 98.85 |
| 0106279 | 0101599 | | | | | | |
| SSR- | SSR- | 58.73 | 59.88 | 59.30 | 85.01 | 72.46 | 78.74 |
| 0106280 | 0101599 | | | | | | |
| SSR- | SSR- | 70.08 | 64.65 | 67.37 | 97.21 | 86.00 | 91.60 |
| 0106281 | 0101599 | | | | | | |
| SSR- | SSR- | 76.71 | 69.28 | 72.99 | 93.72 | 83.25 | 88.48 |
| 0106282 | 0101599 | | | | | | |
| SSR- | SSR- | 82.89 | 81.53 | 82.21 | 98.28 | 94.37 | 96.33 |
| 0106283 | 0101599 | | | | | | |
| SSR- | SSR- | 79.96 | 81.80 | 80.88 | 96.89 | 107.03 | 101.96 |
| 0106284 | 0101599 | | | | | | |
| SSR- | SSR- | 98.91 | 92.59 | 95.75 | 110.51 | 113.56 | 112.04 |
| 0106285 | 0101599 | | | | | | |
| SSR- | SSR- | 76.18 | 81.74 | 78.96 | 88.76 | 82.92 | 85.84 |
| 0106286 | 0101599 | | | | | | |
| SSR- | SSR- | 88.73 | 86.85 | 87.79 | 96.76 | 104.80 | 100.78 |
| 0106287 | 0101599 | | | | | | |
| SSR- | SSR- | 70.00 | 64.80 | 67.40 | 77.55 | 83.51 | 80.53 |
| 0106288 | 0101599 | | | | | | |
| SSR- | SSR- | 95.29 | 95.15 | 95.22 | 106.41 | 109.41 | 107.91 |
| 0106289 | 0101599 | | | | | | |
| SSR- | SSR- | 80.90 | 73.32 | 77.11 | 101.59 | 87.39 | 94.49 |
| 0106290 | 0101599 | | | | | | |
| SSR- | SSR- | 83.20 | 80.52 | 81.86 | 95.17 | 102.12 | 98.64 |
| 0106291 | 0101599 | | | | | | |
| SSR- | SSR- | 80.25 | 72.07 | 76.16 | 89.74 | 90.97 | 90.35 |
| 0106292 | 0101599 | | | | | | |
| SSR- | SSR- | 58.71 | 49.16 | 53.94 | 82.30 | 67.99 | 75.15 |
| 0106293 | 0101599 | | | | | | |
| SSR- | SSR- | 47.10 | 34.88 | 40.99 | 67.35 | 54.49 | 60.92 |
| 0106294 | 0101599 | | | | | | |
| SSR- | SSR- | 53.22 | 46.58 | 49.90 | 73.00 | 70.51 | 71.76 |
| 0106295 | 0101599 | | | | | | |
| SSR- | SSR- | 42.81 | 33.27 | 38.04 | 60.21 | 56.17 | 58.19 |
| 0106296 | 0101599 | | | | | | |
| SSR- | SSR- | 47.67 | 38.22 | 42.94 | 68.02 | 65.28 | 66.65 |
| 0106297 | 0101599 | | | | | | |
| SSR- | SSR- | 51.69 | 51.32 | 51.50 | 81.79 | 68.44 | 75.11 |
| 0106298 | 0101599 | | | | | | |
| SSR- | SSR- | 58.72 | 47.42 | 53.07 | 87.20 | 62.27 | 74.74 |
| 0106299 | 0101599 | | | | | | |
| SSR- | SSR- | 48.41 | 39.91 | 44.16 | 81.10 | 66.15 | 73.62 |
| 0106300 | 0101599 | | | | | | |
| SSR- | SSR- | 53.61 | 41.37 | 47.49 | 79.49 | 60.58 | 70.04 |
| 0106301 | 0101599 | | | | | | |
| SSR- | SSR- | 45.55 | 42.05 | 43.80 | 58.89 | 68.95 | 63.92 |
| 0106302 | 0101599 | | | | | | |
| SSR- | SSR- | 52.21 | 48.96 | 50.58 | 78.58 | 67.93 | 73.25 |
| 0106303 | 0101599 | | | | | | |
| SSR- | SSR- | 44.07 | 38.67 | 41.37 | 58.24 | 68.80 | 63.52 |
| 0106304 | 0101599 | | | | | | |
| SSR- | SSR- | 47.40 | 41.73 | 44.56 | 75.26 | 62.85 | 69.05 |
| 0106305 | 0101599 | | | | | | |
| SSR- | SSR- | 55.08 | 47.51 | 51.29 | 76.38 | 85.70 | 81.04 |
| 0106306 | 0101599 | | | | | | |
| SSR- | SSR- | 57.79 | 45.72 | 51.76 | 91.94 | 60.26 | 76.10 |
| 0104474 | 0101599 | | | | | | |
| SSR- | SSR- | 53.02 | 46.22 | 49.62 | 66.65 | 85.31 | 75.98 |
| 0106307 | 0101599 | | | | | | |
| SSR- | SSR- | 46.39 | 34.31 | 40.35 | 76.22 | 57.87 | 67.04 |
| 0104475 | 0101599 | | | | | | |
| SSR- | SSR- | 56.11 | 54.66 | 55.39 | 70.93 | 79.09 | 75.01 |
| 0104720 | 0101596 | | | | | | |

While various embodiments have been described and illustrated herein, those of ordinary skill in the art will readily envision a variety of other means and/or structures for performing the functions and/or obtaining the results and/or one or more of the advantages described in the present disclosure, and each of such variations and/or modifications is deemed to be included. More generally, those skilled in the art will readily appreciate that all parameters, dimensions, materials, and configurations described herein are meant to be example and that the actual parameters, dimensions, materials, and/or configurations may depend upon the 10 specific application or applications for which the teachings of the present disclosure is/are used. Those skilled in the art will recognize, or be able to ascertain using no more than routine experimentation, many equivalents to the embodiments of the present disclosure. It is, therefore, to be understood that the foregoing embodiments are presented by way of example only and that, within the scope of the appended claims and equivalents thereto, claimed technologies may be practiced otherwise than as specifically described and claimed. In addition, any combination of two or more features, systems, articles, materials, kits, and/or methods, if such features, systems, articles, materials, kits, and/or methods are not mutually inconsistent, is included within the scope of the present disclosure.

What is claimed is:

1. A double-stranded RNAi (dsRNAi) agent comprising a guide strand and a passenger strand wherein:
a) the guide strand is complementary or substantially complementary to a target RNA sequence, and comprises:
i. backbone phosphorothioate chiral centers in Sp configuration between the 3′ terminal nucleotide and the penultimate (N−1) nucleotide and as between the penultimate (N−1) nucleotide and the immediately upstream (N−2) nucleotide,
ii. backbone phosphorothioate chiral centers in Rp, Sp, or alternating configurations between the 5′ terminal (+1) nucleotide and the immediately downstream (+2) nucleotide and between the +2 nucleotide and the immediately downstream (+3) nucleotide;
iii. one or more backbone phosphorothioate chiral centers in Rp or Sp configuration upstream of backbone phosphorothioate chiral centers in Sp configuration between the 3′ terminal nucleotide and the penultimate (N−1) nucleotide and as between the penultimate (N−1) nucleotide and the immediately upstream (N−2) nucleotide; and/or
iv. one or more backbone phosphorothioate chiral centers in Rp or Sp configuration between the 5′ terminal (+1) nucleotide and the immediately downstream (+2) nucleotide and between the (+2) nucleotide and the immediately downstream (+3) nucleotide, as well as between one or both of (a) the (+3) nucleotide and the (+4) nucleotide; and (b) the (+5) nucleotide and the (+6) nucleotide;
b) the guide strand comprises one or more Rp, Sp, or stereorandom non-negatively charged internucleotidic linkage between any two adjacent nucleotides between the second (+2) nucleotide relative to the 5′ terminal nucleotide of the guide strand and the penultimate 3′ (N−1) nucleotide of the guide strand, where N is the 3′ terminal nucleotide;
c) the guide strand comprises a 2′ modification, of the 3′ nucleotide of a nucleotide pair linked by an Rp, Sp, or stereorandom non-negatively charged internucleotidic linkage;
d) the passenger strand comprises one or both of:
i. 0-n Rp, Sp, or stereorandom non-negatively charged internucleotidic linkages, where n is about 1 to 49, and
ii. one or more backbone chiral centers in Rp or Sp configuration,
e) each strand of the dsRNAi agent independently has a length of about 15 to about 49 nucleotides; and
f) the dsRNAi is capable of directing target-specific RNA interference.

2. A chirally controlled oligonucleotide composition comprising double stranded oligonucleotides wherein the guide and passenger strands of the double stranded oligonucleotides are independently characterized by:
i) a common base sequence and length;
ii) a common pattern of backbone linkages; and
iii) a common pattern of backbone chiral centers;
which composition is chirally controlled in that it is enriched, relative to a substantially racemic preparation of guide strands having the same common base sequence and length, for oligonucleotides having a common pattern of chiral centers; and
a) wherein the guide strands are complementary or substantially complementary to a target RNA sequence, and comprise:
i. backbone phosphorothioate chiral centers in Sp configuration between the 3′ terminal nucleotide and the penultimate (N−1) nucleotide and as between the penultimate (N−1) nucleotide and the immediately upstream (N−2) nucleotide,
ii. backbone phosphorothioate chiral centers in Rp, Sp, or alternating configurations between the 5′ terminal (+1) nucleotide and the immediately downstream (+2) nucleotide and between the +2 nucleotide and the immediately downstream (+3) nucleotide;
iii. one or more backbone phosphorothioate chiral centers in Rp or Sp configuration upstream of backbone phosphorothioate chiral centers in Sp configuration between the 3′ terminal nucleotide and the penultimate (N−1) nucleotide and as between the penultimate (N−1) nucleotide and the immediately upstream (N−2) nucleotide; and/or
iv. one or more backbone phosphorothioate chiral centers in Rp or Sp configuration between the 5′ terminal (+1) nucleotide and the immediately downstream (+2) nucleotide and between the (+2) nucleotide and the immediately downstream (+3) nucleotide, as well as between one or both of (a) the (+3) nucleotide and the (+4) nucleotide; and (b) the (+5) nucleotide and the (+6) nucleotide;
b) the guide strand comprises one or more Rp, Sp, or stereorandom non-negatively charged internucleotidic linkage between any two adjacent nucleotides between the second (+2) nucleotide relative to the 5′ terminal nucleotide of the guide strand and the penultimate 3′ (N−1) nucleotide of the guide strand, where N is the 3′ terminal nucleotide;
c) the guide strand comprises a 2′ modification, of the 3′ nucleotide of a nucleotide pair linked by an Rp, Sp, or stereorandom non-negatively charged internucleotidic linkage;
d) the passenger strands comprise one or both of:
i. 0-n Rp, Sp, or stereorandom non-negatively charged internucleotidic linkages, where n is about 1 to 49, and
ii. one or more backbone chiral centers in Rp or Sp configuration,
e) the guide and passenger strands have a length of about 15 to about 49 nucleotides; and
f) the guide and passenger strands are capable of directing target-specific RNA interference.

3. The double stranded oligonucleotide of claim 1, wherein the guide strand comprises a 5′ terminal modification selected from: 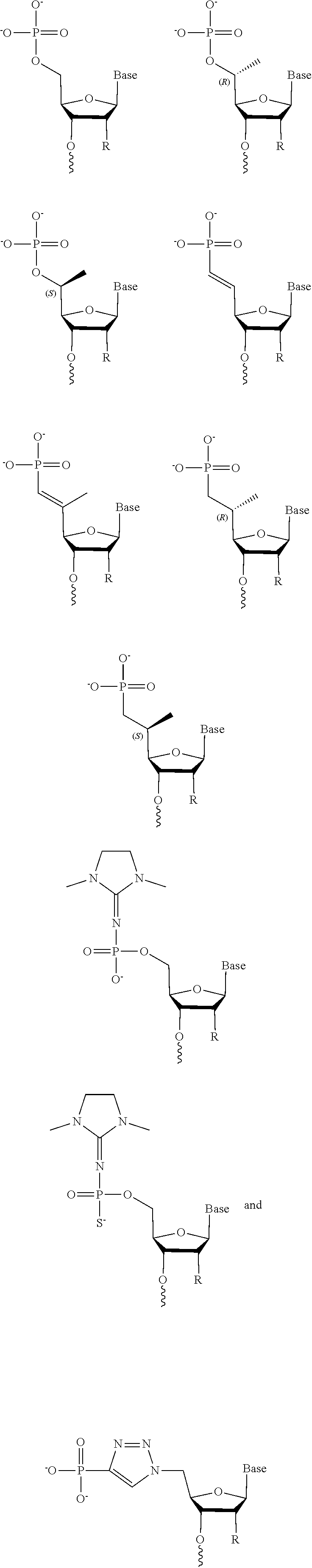
Base: A, C, G, T, U, abasic, and modified nucleobases;
R: H, OH, O-alkyl, F, MOE, LNA bridge to the 4′ position, BNA bridge to the 4′ position.

4. The double stranded oligonucleotide of claim 1, wherein the Rp, Sp, or stereorandom non-negatively charged backbone internucleotidic linkages have neutral charge.

5. The double stranded oligonucleotide or composition of claim 17, wherein the neutral backbone internucleotidic linkages is

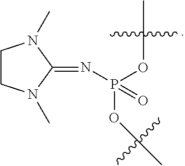 [SMILES: *OP(=O)(N=C1N(C)CCN1C)O*]

6. The composition of claim 2, where the guide and passenger strands in the composition that independently share a common base sequence, a common pattern of base modification, a common pattern of sugar modification, and/or a common pattern of internucleotidic linkages are at least 90% of all the guide and passenger strands in the composition.

7. The double stranded oligonucleotide of claim 1, wherein the double stranded oligonucleotide comprises a carbohydrate moiety, lipid moiety, or a target moiety connected at a nucleoside or an internucleotidic linkage, optionally through a linker.

8. The double stranded oligonucleotide of claim 1, wherein at least 20%, 25%, 30%, 35%, 40%, 45%, 50%, 55%, 60%, 65%, 70%, 75%, 80%, 85%, 90% or 95% of the internucleotidic linkages of the double stranded oligonucleotide are independently chiral internucleotidic linkages.

9. The double stranded oligonucleotide of claim 1, wherein at least 3%, 5%, 10%, 15%, 20%, 25%, 30%, 35%, 40%, 45%, 50%, 55%, 60%, 65%, 70%, 75%, 80%, 85%, 90%, 95%, or 97% of the nucleotidic units of the double stranded oligonucleotide independently comprise a 2′-substitution.

10. The double stranded oligonucleotide of claim 1, wherein a 2′-substitution of the oligonucleotide is 2′-F.

11. The double stranded oligonucleotide of claim 1, wherein a 2′-substitution of the oligonucleotide is 2′-OR¹, wherein R¹ is optionally substituted C₁₋₁₀ aliphatic.

12. The double stranded oligonucleotide of claim 1, wherein a 2′-substitution of the oligonucleotide is -L-, wherein L connects C2 and C4 of the sugar unit.

13. The double stranded oligonucleotide of claim 1, wherein the guide strand comprises a target-binding sequence that is completely complementary to a target sequence, wherein the target-binding sequence has a length of at least 10, 11, 12, 13, 14, 15, 16, 17, 18, 19 or 20 bases, wherein each base is optionally substituted adenine, cytosine, guanosine, thymine, or uracil, and wherein the target sequence comprises one or more allelic sites, wherein an allelic site is a SNP or a mutation.

14. The double stranded oligonucleotide of claim 1, wherein the target sequence comprises an allelic site and the target-binding sequence is completely complementary to the target sequence of a disease-associated allele but not that of an allele less associated with the disease.

15. The double stranded oligonucleotide of claim 1, wherein
the double stranded oligonucleotide comprises a guide strand that binds with a transcript of a target nucleic acid sequence for which a plurality of alleles exist within a population, each of which contains a specific nucleotide characteristic sequence element that defines the allele relative to other alleles of the same target nucleic acid sequence,
wherein the base sequence of the guide strand is or comprises a sequence that is complementary to the characteristic sequence element that defines a particular allele, and
the guide strand being characterized in that, when it is contacted with a cell comprising transcripts of target nucleic acid sequence, it shows suppression of transcripts of the particular allele, or a protein encoded thereby, at a level that is greater than a level of suppression observed for another allele of the same nucleic acid sequence.

16. The double stranded oligonucleotide of claim 1, wherein the passenger strand comprises:
an Sp backbone phosphorothioate chiral center between the 5′ terminal (+1) nucleotide and the immediately downstream (+2) nucleotide; and
an Sp backbone phosphorothioate chiral center between the penultimate (N−1) nucleotide and the 3′ terminal (N) nucleotide.

17. A method for reducing level and/or activity of a transcript or a protein encoded thereby, comprising administering to a cell expressing the transcript a double stranded oligonucleotide, wherein the double stranded oligonucleotide comprises a guide strand and a passenger strand wherein:
a) the guide strand is complementary or substantially complementary to a target RNA sequence, and comprises:
i. backbone phosphorothioate chiral centers in Sp configuration between the 3′ terminal nucleotide and the penultimate (N−1) nucleotide and as between the penultimate (N−1) nucleotide and the immediately upstream (N−2) nucleotide,
ii. backbone phosphorothioate chiral centers in Rp, Sp, or alternating configurations between the 5′ terminal (+1) nucleotide and the immediately downstream (+2) nucleotide and between the +2 nucleotide and the immediately downstream (+3) nucleotide;
iii. one or more backbone phosphorothioate chiral centers in Rp or Sp configuration upstream of backbone phosphorothioate chiral centers in Sp configuration between the 3′ terminal nucleotide and the penultimate (N−1) nucleotide and as between the penultimate (N−1) nucleotide and the immediately upstream (N−2) nucleotide; and/or
iv. one or more backbone phosphorothioate chiral centers in Rp or Sp configuration between the 5′ terminal (+1) nucleotide and the immediately downstream (+2) nucleotide and between the (+2) nucleotide and the immediately downstream (+3) nucleotide, as well as between one or both of: (a) the (+3) nucleotide and the (+4) nucleotide; and (b) the (+5) nucleotide and the (+6) nucleotide;
b) the guide strand comprises one or more Rp, Sp, or stereorandom non-negatively charged internucleotidic linkage between any two adjacent nucleotides between the second (+2) nucleotide relative to the 5′ terminal nucleotide of the guide strand and the penultimate 3′ (N−1) nucleotide of the guide strand, where N is the 3′ terminal nucleotide;
c) the guide strand comprises a 2′ modification, of the 3′ nucleotide of a nucleotide pair linked by an Rp, Sp, or stereorandom non-negatively charged internucleotidic linkage;
d) the passenger strand comprises one or both of:
i. 0-n Rp, Sp, or stereorandom non-negatively charged internucleotidic linkages, where n is about 1 to 49, and
ii. one or more backbone chiral centers in Rp or Sp configuration,
e) each strand of the dsRNAi agent independently has a length of about 15 to about 49 nucleotides; and
f) the dsRNAi is capable of directing target-specific RNA interference; and
wherein the guide strand of double stranded oligonucleotide or composition comprises a targeting-binding sequence that is completely complementary to a target sequence in the transcript.

18. The method of claim 17, wherein the cell is an immune cell, a blood cell, a cardiac cell, a lung cell, an optic cell, a muscle cell, a liver cell, a kidney cell, a brain cell, a cell of the central nervous system, or a cell of the peripheral nervous system.

19. The method of claim 17, for allele-specific suppression of a transcript from a nucleic acid sequence for which a plurality of alleles exist within a population, each of which contains a specific nucleotide characteristic sequence element that defines the allele relative to other alleles of the same target nucleic acid sequence, the method comprising steps of:
a) contacting a sample comprising transcripts of the target nucleic acid sequence with the double stranded oligonucleotide
wherein the guide strand of the double stranded oligonucleotide or composition comprises a targeting-binding sequence that is identical or completely complementary to a target sequence in the nucleic acid sequence, which target sequence comprises a characteristic sequence element that defines a particular allele, and
wherein when the guide strand of the double stranded oligonucleotide or composition is contacted with a cell comprising transcripts of both the target allele and another allele of the same nucleic acid sequence, transcripts of the particular allele are suppressed at a greater level than a level of suppression observed for another allele of the same nucleic acid sequence.

20. The method of claim 17, for allele-specific suppression of a transcript from a nucleic acid sequence for which a plurality of alleles exist within a population, each of which contains a specific nucleotide characteristic sequence element that defines the allele relative to other alleles of the same target nucleic acid sequence, the method comprising steps of:
administering to a subject comprising transcripts of the target nucleic acid sequence with the double stranded oligonucleotide
wherein the guide strand of the double stranded oligonucleotide or composition comprises a targeting-binding sequence that is identical or completely complementary to a target sequence in the nucleic acid sequence, which target sequence comprises a characteristic sequence element that defines a particular allele, and
wherein when the guide strand of the double stranded oligonucleotide or composition is contacted with a cell comprising transcripts of both the target allele and another allele of the same nucleic acid sequence, transcripts of the particular allele are suppressed at a greater level than a level of suppression observed for another allele of the same nucleic acid sequence.

21. The method of claim 17, wherein when the oligonucleotide or oligonucleotide of the composition is contacted with a cell comprising transcripts of both the target allele and another allele of the same nucleic acid sequence, it shows suppression of transcripts of the particular allele at a level that is:
a) greater than when the composition is absent;
b) greater than a level of suppression observed for another allele of the same nucleic acid sequence; or
c) both greater than when the composition is absent, and greater than a level of suppression observed for another allele of the same nucleic acid sequence.


SEQUENCE LISTING (736 sequences)

SEQ ID NO: 1 - RNA, 23 nt, synthetic construct
  misc_feature 1..23: note = Synthetic
  source 1..23: mol_type = other RNA
ttatagagcaagaacactgtttt

SEQ ID NO: 2 - RNA, 23 nt, synthetic construct
  misc_feature 1..23: note = Synthetic
  source 1..23: mol_type = other RNA
ttatagagcaagaacactgtttt

SEQ ID NO: 3 - RNA, 23 nt, synthetic construct
  misc_feature 1..23: note = Synthetic
  source 1..23: mol_type = other RNA
ttatagagcaagaacactgtttt

SEQ ID NO: 4 - RNA, 23 nt, synthetic construct
  misc_feature 1..23: note = Synthetic
  source 1..23: mol_type = other RNA
ttatagagcaagaacactgtttt

SEQ ID NO: 5 - RNA, 23 nt, synthetic construct
  misc_feature 1..23: note = Synthetic
  source 1..23: mol_type = other RNA
ttatagagcaagaacactgtttt

SEQ ID NO: 6 - RNA, 23 nt, synthetic construct
  misc_feature 1..23: note = Synthetic
  source 1..23: mol_type = other RNA
ttatagagcaagaacactgtttt

SEQ ID NO: 7 - RNA, 23 nt, synthetic construct
  misc_feature 1..23: note = Synthetic
  source 1..23: mol_type = other RNA
ttatagagcaagaacactgtttt

SEQ ID NO: 8 - RNA, 23 nt, synthetic construct
  misc_feature 1..23: note = Synthetic
  source 1..23: mol_type = other RNA
ttatagagcaagaacactgtttt

SEQ ID NO: 9 - RNA, 23 nt, synthetic construct
  misc_feature 1..23: note = Synthetic
  source 1..23: mol_type = other RNA
ttatagagcaagaacactgtttt

SEQ ID NO: 10 - RNA, 23 nt, synthetic construct
  misc_feature 1..23: note = Synthetic
  source 1..23: mol_type = other RNA
ttatagagcaagaacactgtttt

SEQ ID NO: 11 - RNA, 23 nt, synthetic construct
  misc_feature 1..23: note = Synthetic
  source 1..23: mol_type = other RNA
ttatagagcaagaacactgtttt

SEQ ID NO: 12 - RNA, 23 nt, synthetic construct
  misc_feature 1..23: note = Synthetic
  source 1..23: mol_type = other RNA
ttatagagcaagaacactgtttt

SEQ ID NO: 13 - RNA, 23 nt, synthetic construct
  misc_feature 1..23: note = Synthetic
  source 1..23: mol_type = other RNA
ttatagagcaagaacactgtttt

SEQ ID NO: 14 - RNA, 23 nt, synthetic construct
  misc_feature 1..23: note = Synthetic
  source 1..23: mol_type = other RNA
ttatagagcaagaacactgtttt

SEQ ID NO: 15 - RNA, 23 nt, synthetic construct
  misc_feature 1..23: note = Synthetic
  source 1..23: mol_type = other RNA
ttatagagcaagaacactgtttt

SEQ ID NO: 16 - RNA, 23 nt, synthetic construct
  misc_feature 1..23: note = Synthetic
  source 1..23: mol_type = other RNA
ttatagagcaagaacactgtttt

SEQ ID NO: 17 - RNA, 23 nt, synthetic construct
  misc_feature 1..23: note = Synthetic
  source 1..23: mol_type = other RNA
ttatagagcaagaacactgtttt

SEQ ID NO: 18 - RNA, 23 nt, synthetic construct
  misc_feature 1..23: note = Synthetic
  source 1..23: mol_type = other RNA
ttatagagcaagaacactgtttt

SEQ ID NO: 19 - RNA, 23 nt, synthetic construct
  misc_feature 1..23: note = Synthetic
  source 1..23: mol_type = other RNA
ttatagagcaagaacactgtttt

SEQ ID NO: 20 - RNA, 23 nt, synthetic construct
  misc_feature 1..23: note = Synthetic
  source 1..23: mol_type = other RNA
ttatagagcaagaacactgtttt

SEQ ID NO: 21 - RNA, 23 nt, synthetic construct
  misc_feature 1..23: note = Synthetic
  source 1..23: mol_type = other RNA
ttatagagcaagaacactgtttt

SEQ ID NO: 22 - RNA, 23 nt, synthetic construct
  misc_feature 1..23: note = Synthetic
  source 1..23: mol_type = other RNA
ttatagagcaagaacactgtttt

SEQ ID NO: 23 - RNA, 23 nt, synthetic construct
  misc_feature 1..23: note = Synthetic
  source 1..23: mol_type = other RNA
ttatagagcaagaacactgtttt

SEQ ID NO: 24 - RNA, 23 nt, synthetic construct
  misc_feature 1..23: note = Synthetic
  source 1..23: mol_type = other RNA
ttatagagcaagaacactgtttt

SEQ ID NO: 25 - RNA, 23 nt, synthetic construct
  misc_feature 1..23: note = Synthetic
  source 1..23: mol_type = other RNA
ttatagagcaagaacactgtttt

SEQ ID NO: 26 - RNA, 23 nt, synthetic construct
  misc_feature 1..23: note = Synthetic
  source 1..23: mol_type = other RNA
ttatagagcaagaacactgtttt

SEQ ID NO: 27 - RNA, 23 nt, synthetic construct
  misc_feature 1..23: note = Synthetic
  source 1..23: mol_type = other RNA
ttatagagcaagaacactgtttt

SEQ ID NO: 28 - RNA, 23 nt, synthetic construct
  misc_feature 1..23: note = Synthetic
  source 1..23: mol_type = other RNA
ttatagagcaagaacactgtttt

SEQ ID NO: 29 - RNA, 23 nt, synthetic construct
  misc_feature 1..23: note = Synthetic
  source 1..23: mol_type = other RNA
ttatagagcaagaacactgtttt

SEQ ID NO: 30 - RNA, 23 nt, synthetic construct
  misc_feature 1..23: note = Synthetic
  source 1..23: mol_type = other RNA
ttatagagcaagaacactgtttt

SEQ ID NO: 31 - RNA, 23 nt, synthetic construct
  misc_feature 1..23: note = Synthetic
  source 1..23: mol_type = other RNA
ttatagagcaagaacactgtttt

SEQ ID NO: 32 - RNA, 23 nt, synthetic construct
  misc_feature 1..23: note = Synthetic
  source 1..23: mol_type = other RNA
ttatagagcaagaacactgtttt

SEQ ID NO: 33 - RNA, 23 nt, synthetic construct
  misc_feature 1..23: note = Synthetic
  source 1..23: mol_type = other RNA
ttatagagcaagaacactgtttt

SEQ ID NO: 34 - RNA, 23 nt, synthetic construct
  misc_feature 1..23: note = Synthetic
  source 1..23: mol_type = other RNA
ttatagagcaagaacactgtttt

SEQ ID NO: 35 - RNA, 23 nt, synthetic construct
  misc_feature 1..23: note = Synthetic
  source 1..23: mol_type = other RNA
ttatagagcaagaacactgtttt

SEQ ID NO: 36 - RNA, 23 nt, synthetic construct
  misc_feature 1..23: note = Synthetic
  source 1..23: mol_type = other RNA
ttatagagcaagaacactgtttt

SEQ ID NO: 37 - RNA, 23 nt, synthetic construct
  misc_feature 1..23: note = Synthetic
  source 1..23: mol_type = other RNA
ttatagagcaagaacactgtttt

SEQ ID NO: 38 - RNA, 23 nt, synthetic construct
  misc_feature 1..23: note = Synthetic
  source 1..23: mol_type = other RNA
ttatagagcaagaacactgtttt

SEQ ID NO: 39 - RNA, 23 nt, synthetic construct
  misc_feature 1..23: note = Synthetic
  source 1..23: mol_type = other RNA
ttatagagcaagaacactgtttt

SEQ ID NO: 40 - RNA, 23 nt, synthetic construct
  misc_feature 1..23: note = Synthetic
  source 1..23: mol_type = other RNA
ttatagagcaagaacactgtttt

SEQ ID NO: 41 - RNA, 23 nt, synthetic construct
  misc_feature 1..23: note = Synthetic
  source 1..23: mol_type = other RNA
ttatagagcaagaacactgtttt

SEQ ID NO: 42 - RNA, 23 nt, synthetic construct
  misc_feature 1..23: note = Synthetic
  source 1..23: mol_type = other RNA
ttatagagcaagaacactgtttt

SEQ ID NO: 43 - RNA, 23 nt, synthetic construct
  misc_feature 1..23: note = Synthetic
  source 1..23: mol_type = other RNA
ttatagagcaagaacactgtttt

SEQ ID NO: 44 - RNA, 23 nt, synthetic construct
  misc_feature 1..23: note = Synthetic
  source 1..23: mol_type = other RNA
ttatagagcaagaacactgtttt

SEQ ID NO: 45 - RNA, 23 nt, synthetic construct
  misc_feature 1..23: note = Synthetic
  source 1..23: mol_type = other RNA
ttatagagcaagaacactgtttt

SEQ ID NO: 46 - RNA, 23 nt, synthetic construct
  misc_feature 1..23: note = Synthetic
  source 1..23: mol_type = other RNA
ttatagagcaagaacactgtttt

SEQ ID NO: 47 - RNA, 23 nt, synthetic construct
  misc_feature 1..23: note = Synthetic
  source 1..23: mol_type = other RNA
ttatagagcaagaacactgtttt

SEQ ID NO: 48 - RNA, 23 nt, synthetic construct
  misc_feature 1..23: note = Synthetic
  source 1..23: mol_type = other RNA
ttatagagcaagaacactgtttt

SEQ ID NO: 49 - RNA, 23 nt, synthetic construct
  misc_feature 1..23: note = Synthetic
  source 1..23: mol_type = other RNA
ttatagagcaagaacactgtttt

SEQ ID NO: 50 - RNA, 23 nt, synthetic construct
  misc_feature 1..23: note = Synthetic
  source 1..23: mol_type = other RNA
ttatagagcaagaacactgtttt

SEQ ID NO: 51 - RNA, 23 nt, synthetic construct
  misc_feature 1..23: note = Synthetic
  source 1..23: mol_type = other RNA
ttatagagcaagaacactgtttt

SEQ ID NO: 52 - RNA, 23 nt, synthetic construct
  misc_feature 1..23: note = Synthetic
  source 1..23: mol_type = other RNA
ttatagagcaagaacactgtttt

SEQ ID NO: 53 - RNA, 23 nt, synthetic construct
  misc_feature 1..23: note = Synthetic
  source 1..23: mol_type = other RNA
ttatagagcaagaacactgtttt

SEQ ID NO: 54 - RNA, 23 nt, synthetic construct
  misc_feature 1..23: note = Synthetic
  source 1..23: mol_type = other RNA
ttatagagcaagaacactgtttt

SEQ ID NO: 55 - RNA, 23 nt, synthetic construct
  misc_feature 1..23: note = Synthetic
  source 1..23: mol_type = other RNA
ttatagagcaagaacactgtttt

SEQ ID NO: 56 - RNA, 23 nt, synthetic construct
  misc_feature 1..23: note = Synthetic
  source 1..23: mol_type = other RNA
ttatagagcaagaacactgtttt

SEQ ID NO: 57 - RNA, 23 nt, synthetic construct
  misc_feature 1..23: note = Synthetic
  source 1..23: mol_type = other RNA
ttatagagcaagaacactgtttt

SEQ ID NO: 58 - RNA, 23 nt, synthetic construct
  misc_feature 1..23: note = Synthetic
  source 1..23: mol_type = other RNA
ttatagagcaagaacactgtttt

SEQ ID NO: 59 - RNA, 23 nt, synthetic construct
  misc_feature 1..23: note = Synthetic
  source 1..23: mol_type = other RNA
ttatagagcaagaacactgtttt

SEQ ID NO: 60 - RNA, 23 nt, synthetic construct
  misc_feature 1..23: note = Synthetic
  source 1..23: mol_type = other RNA
ttatagagcaagaacactgtttt

SEQ ID NO: 61 - RNA, 23 nt, synthetic construct
  misc_feature 1..23: note = Synthetic
  source 1..23: mol_type = other RNA
ttatagagcaagaacactgtttt

SEQ ID NO: 62 - RNA, 23 nt, synthetic construct
  misc_feature 1..23: note = Synthetic
  source 1..23: mol_type = other RNA
ttatagagcaagaacactgtttt

SEQ ID NO: 63 - RNA, 23 nt, synthetic construct
  misc_feature 1..23: note = Synthetic
  source 1..23: mol_type = other RNA
ttatagagcaagaacactgtttt

SEQ ID NO: 64 - RNA, 23 nt, synthetic construct
  misc_feature 1..23: note = Synthetic
  source 1..23: mol_type = other RNA
ttatagagcaagaacactgtttt

SEQ ID NO: 65 - RNA, 23 nt, synthetic construct
  misc_feature 1..23: note = Synthetic
  source 1..23: mol_type = other RNA
ttatagagcaagaacactgtttt

SEQ ID NO: 66 - RNA, 23 nt, synthetic construct
  misc_feature 1..23: note = Synthetic
  source 1..23: mol_type = other RNA
ttatagagcaagaacactgtttt

SEQ ID NO: 67 - RNA, 23 nt, synthetic construct
  misc_feature 1..23: note = Synthetic
  source 1..23: mol_type = other RNA
ttatagagcaagaacactgtttt

SEQ ID NO: 68 - RNA, 23 nt, synthetic construct
  misc_feature 1..23: note = Synthetic
  source 1..23: mol_type = other RNA
ttatagagcaagaacactgtttt

SEQ ID NO: 69 - RNA, 23 nt, synthetic construct
  misc_feature 1..23: note = Synthetic
  source 1..23: mol_type = other RNA
ttatagagcaagaacactgtttt

SEQ ID NO: 70 - RNA, 23 nt, synthetic construct
  misc_feature 1..23: note = Synthetic
  source 1..23: mol_type = other RNA
ttatagagcaagaacactgtttt

SEQ ID NO: 71 - RNA, 23 nt, synthetic construct
  misc_feature 1..23: note = Synthetic
  source 1..23: mol_type = other RNA
ttatagagcaagaacactgtttt

SEQ ID NO: 72 - RNA, 23 nt, synthetic construct
  misc_feature 1..23: note = Synthetic
  source 1..23: mol_type = other RNA
ttatagagcaagaacactgtttt

SEQ ID NO: 73 - RNA, 23 nt, synthetic construct
  misc_feature 1..23: note = Synthetic
  source 1..23: mol_type = other RNA
ttatagagcaagaacactgtttt

SEQ ID NO: 74 - RNA, 23 nt, synthetic construct
  misc_feature 1..23: note = Synthetic
  source 1..23: mol_type = other RNA
ttatagagcaagaacactgtttt

SEQ ID NO: 75 - RNA, 23 nt, synthetic construct
  misc_feature 1..23: note = Synthetic
  source 1..23: mol_type = other RNA
ttatagagcaagaacactgtttt

SEQ ID NO: 76 - RNA, 23 nt, synthetic construct
  misc_feature 1..23: note = Synthetic
  source 1..23: mol_type = other RNA
ttatagagcaagaacactgtttt

SEQ ID NO: 77 - RNA, 23 nt, synthetic construct
  misc_feature 1..23: note = Synthetic
  source 1..23: mol_type = other RNA
ttatagagcaagaacactgtttt

SEQ ID NO: 78 - RNA, 23 nt, synthetic construct
  misc_feature 1..23: note = Synthetic
  source 1..23: mol_type = other RNA
ttatagagcaagaacactgtttt

SEQ ID NO: 79 - RNA, 23 nt, synthetic construct
  misc_feature 1..23: note = Synthetic
  source 1..23: mol_type = other RNA
ttatagagcaagaacactgtttt

SEQ ID NO: 80 - RNA, 23 nt, synthetic construct
  misc_feature 1..23: note = Synthetic
  source 1..23: mol_type = other RNA
ttatagagcaagaacactgtttt

SEQ ID NO: 81 - RNA, 23 nt, synthetic construct
  misc_feature 1..23: note = Synthetic
  source 1..23: mol_type = other RNA
ttatagagcaagaacactgtttt

SEQ ID NO: 82 - RNA, 23 nt, synthetic construct
  misc_feature 1..23: note = Synthetic
  source 1..23: mol_type = other RNA
ttatagagcaagaacactgtttt

SEQ ID NO: 83 - RNA, 23 nt, synthetic construct
  misc_feature 1..23: note = Synthetic
  source 1..23: mol_type = other RNA
ttatagagcaagaacactgtttt

SEQ ID NO: 84 - RNA, 23 nt, synthetic construct
  misc_feature 1..23: note = Synthetic
  source 1..23: mol_type = other RNA
ttatagagcaagaacactgtttt

SEQ ID NO: 85 - RNA, 23 nt, synthetic construct
  misc_feature 1..23: note = Synthetic
  source 1..23: mol_type = other RNA
ttatagagcaagaacactgtttt

SEQ ID NO: 86 - RNA, 23 nt, synthetic construct
  misc_feature 1..23: note = Synthetic
  source 1..23: mol_type = other RNA
ttatagagcaagaacactgtttt

SEQ ID NO: 87 - RNA, 23 nt, synthetic construct
  misc_feature 1..23: note = Synthetic
  source 1..23: mol_type = other RNA
ttatagagcaagaacactgtttt

SEQ ID NO: 88 - RNA, 23 nt, synthetic construct
  misc_feature 1..23: note = Synthetic
  source 1..23: mol_type = other RNA
ttatagagcaagaacactgtttt

SEQ ID NO: 89 - RNA, 23 nt, synthetic construct
  misc_feature 1..23: note = Synthetic
  source 1..23: mol_type = other RNA
ttatagagcaagaacactgtttt

SEQ ID NO: 90 - RNA, 23 nt, synthetic construct
  misc_feature 1..23: note = Synthetic
  source 1..23: mol_type = other RNA
ttatagagcaagaacactgtttt

SEQ ID NO: 91 - RNA, 23 nt, synthetic construct
  misc_feature 1..23: note = Synthetic
  source 1..23: mol_type = other RNA
ttatagagcaagaacactgtttt

SEQ ID NO: 92 - RNA, 23 nt, synthetic construct
  misc_feature 1..23: note = Synthetic
  source 1..23: mol_type = other RNA
ttatagagcaagaacactgtttt

SEQ ID NO: 93 - RNA, 23 nt, synthetic construct
  misc_feature 1..23: note = Synthetic
  source 1..23: mol_type = other RNA
ttatagagcaagaacactgtttt

SEQ ID NO: 94 - RNA, 23 nt, synthetic construct
  misc_feature 1..23: note = Synthetic
  source 1..23: mol_type = other RNA
ttatagagcaagaacactgtttt

SEQ ID NO: 95 - RNA, 23 nt, synthetic construct
  misc_feature 1..23: note = Synthetic
  source 1..23: mol_type = other RNA
ttatagagcaagaacactgtttt

SEQ ID NO: 96 - RNA, 23 nt, synthetic construct
  misc_feature 1..23: note = Synthetic
  source 1..23: mol_type = other RNA
ttatagagcaagaacactgtttt

SEQ ID NO: 97 - RNA, 23 nt, synthetic construct
  misc_feature 1..23: note = Synthetic
  source 1..23: mol_type = other RNA
ttatagagcaagaacactgtttt

SEQ ID NO: 98 - RNA, 23 nt, synthetic construct
  misc_feature 1..23: note = Synthetic
  source 1..23: mol_type = other RNA
ttatagagcaagaacactgtttt

SEQ ID NO: 99 - RNA, 23 nt, synthetic construct
  misc_feature 1..23: note = Synthetic
  source 1..23: mol_type = other RNA
ttatagagcaagaacactgtttt

SEQ ID NO: 100 - RNA, 23 nt, synthetic construct
  misc_feature 1..23: note = Synthetic
  source 1..23: mol_type = other RNA
ttatagagcaagaacactgtttt

SEQ ID NO: 101 - RNA, 23 nt, synthetic construct
  misc_feature 1..23: note = Synthetic
  source 1..23: mol_type = other RNA
ttatagagcaagaacactgtttt

SEQ ID NO: 102 - RNA, 23 nt, synthetic construct
  misc_feature 1..23: note = Synthetic
  source 1..23: mol_type = other RNA
ttatagagcaagaacactgtttt

SEQ ID NO: 103 - RNA, 23 nt, synthetic construct
  misc_feature 1..23: note = Synthetic
  source 1..23: mol_type = other RNA
ttatagagcaagaacactgtttt

SEQ ID NO: 104 - RNA, 23 nt, synthetic construct
  misc_feature 1..23: note = Synthetic
  source 1..23: mol_type = other RNA
ttatagagcaagaacactgtttt

SEQ ID NO: 105 - RNA, 23 nt, synthetic construct
  misc_feature 1..23: note = Synthetic
  source 1..23: mol_type = other RNA
ttatagagcaagaacactgtttt

SEQ ID NO: 106 - RNA, 23 nt, synthetic construct
  misc_feature 1..23: note = Synthetic
  source 1..23: mol_type = other RNA
ttatagagcaagaacactgtttt

SEQ ID NO: 107 - RNA, 23 nt, synthetic construct
  misc_feature 1..23: note = Synthetic
  source 1..23: mol_type = other RNA
ttatagagcaagaacactgtttt

SEQ ID NO: 108 - RNA, 23 nt, synthetic construct
  misc_feature 1..23: note = Synthetic
  source 1..23: mol_type = other RNA
ttatagagcaagaacactgtttt

SEQ ID NO: 109 - RNA, 23 nt, synthetic construct
  misc_feature 1..23: note = Synthetic
  source 1..23: mol_type = other RNA
ttatagagcaagaacactgtttt

SEQ ID NO: 110 - RNA, 23 nt, synthetic construct
  misc_feature 1..23: note = Synthetic
  source 1..23: mol_type = other RNA
ttatagagcaagaacactgtttt

SEQ ID NO: 111 - RNA, 23 nt, synthetic construct
  misc_feature 1..23: note = Synthetic
  source 1..23: mol_type = other RNA
ttatagagcaagaacactgtttt

SEQ ID NO: 112 - RNA, 23 nt, synthetic construct
  misc_feature 1..23: note = Synthetic
  source 1..23: mol_type = other RNA
ttatagagcaagaacactgtttt

SEQ ID NO: 113 - RNA, 23 nt, synthetic construct
  misc_feature 1..23: note = Synthetic
  source 1..23: mol_type = other RNA
ttatagagcaagaacactgtttt

SEQ ID NO: 114 - RNA, 23 nt, synthetic construct
  misc_feature 1..23: note = Synthetic
  source 1..23: mol_type = other RNA
ttatagagcaagaacactgtttt

SEQ ID NO: 115 - RNA, 23 nt, synthetic construct
  misc_feature 1..23: note = Synthetic
  source 1..23: mol_type = other RNA
ttatagagcaagaacactgtttt

SEQ ID NO: 116 - RNA, 23 nt, synthetic construct
  misc_feature 1..23: note = Synthetic
  source 1..23: mol_type = other RNA
ttatagagcaagaacactgtttt

SEQ ID NO: 117 - RNA, 23 nt, synthetic construct
  misc_feature 1..23: note = Synthetic
  source 1..23: mol_type = other RNA
ttatagagcaagaacactgtttt

SEQ ID NO: 118 - RNA, 23 nt, synthetic construct
  misc_feature 1..23: note = Synthetic
  source 1..23: mol_type = other RNA
ttatagagcaagaacactgtttt

SEQ ID NO: 119 - RNA, 23 nt, synthetic construct
  misc_feature 1..23: note = Synthetic
  source 1..23: mol_type = other RNA
ttatagagcaagaacactgtttt

SEQ ID NO: 120 - RNA, 23 nt, synthetic construct
  misc_feature 1..23: note = Synthetic
  source 1..23: mol_type = other RNA
ttatagagcaagaacactgtttt

SEQ ID NO: 121 - RNA, 23 nt, synthetic construct
  misc_feature 1..23: note = Synthetic
  source 1..23: mol_type = other RNA
ttatagagcaagaacactgtttt

SEQ ID NO: 122 - RNA, 23 nt, synthetic construct
  misc_feature 1..23: note = Synthetic
  source 1..23: mol_type = other RNA
ttatagagcaagaacactgtttt

SEQ ID NO: 123 - RNA, 23 nt, synthetic construct
  misc_feature 1..23: note = Synthetic
  source 1..23: mol_type = other RNA
ttatagagcaagaacactgtttt

SEQ ID NO: 124 - RNA, 23 nt, synthetic construct
  misc_feature 1..23: note = Synthetic
  source 1..23: mol_type = other RNA
ttatagagcaagaacactgtttt

SEQ ID NO: 125 - RNA, 23 nt, synthetic construct
  misc_feature 1..23: note = Synthetic
  source 1..23: mol_type = other RNA
ttatagagcaagaacactgtttt

SEQ ID NO: 126 - RNA, 23 nt, synthetic construct
  misc_feature 1..23: note = Synthetic
  source 1..23: mol_type = other RNA
ttatagagcaagaacactgtttt

SEQ ID NO: 127 - RNA, 23 nt, synthetic construct
  misc_feature 1..23: note = Synthetic
  source 1..23: mol_type = other RNA
ttatagagcaagaacactgtttt

SEQ ID NO: 128 - RNA, 23 nt, synthetic construct
  misc_feature 1..23: note = Synthetic
  source 1..23: mol_type = other RNA
ttatagagcaagaacactgtttt

SEQ ID NO: 129 - RNA, 23 nt, synthetic construct
  misc_feature 1..23: note = Synthetic
  source 1..23: mol_type = other RNA
ttatagagcaagaacactgtttt

SEQ ID NO: 130 - RNA, 23 nt, synthetic construct
  misc_feature 1..23: note = Synthetic
  source 1..23: mol_type = other RNA
ttatagagcaagaacactgtttt

SEQ ID NO: 131 - RNA, 23 nt, synthetic construct
  misc_feature 1..23: note = Synthetic
  source 1..23: mol_type = other RNA
ttatagagcaagaacactgtttt

SEQ ID NO: 132 - RNA, 23 nt, synthetic construct
  misc_feature 1..23: note = Synthetic
  source 1..23: mol_type = other RNA
ttatagagcaagaacactgtttt

SEQ ID NO: 133 - RNA, 23 nt, synthetic construct
  misc_feature 1..23: note = Synthetic
  source 1..23: mol_type = other RNA
ttatagagcaagaacactgtttt

SEQ ID NO: 134 - RNA, 23 nt, synthetic construct
  misc_feature 1..23: note = Synthetic
  source 1..23: mol_type = other RNA
ttatagagcaagaacactgtttt

SEQ ID NO: 135 - RNA, 23 nt, synthetic construct
  misc_feature 1..23: note = Synthetic
  source 1..23: mol_type = other RNA
ttatagagcaagaacactgtttt

SEQ ID NO: 136 - RNA, 23 nt, synthetic construct
  misc_feature 1..23: note = Synthetic
  source 1..23: mol_type = other RNA
ttatagagcaagaacactgtttt

SEQ ID NO: 137 - RNA, 23 nt, synthetic construct
  misc_feature 1..23: note = Synthetic
  source 1..23: mol_type = other RNA
ttatagagcaagaacactgtttt

SEQ ID NO: 138 - RNA, 23 nt, synthetic construct
  misc_feature 1..23: note = Synthetic
  source 1..23: mol_type = other RNA
ttatagagcaagaacactgtttt

SEQ ID NO: 139 - RNA, 23 nt, synthetic construct
  misc_feature 1..23: note = Synthetic
  source 1..23: mol_type = other RNA
ttatagagcaagaacactgtttt

SEQ ID NO: 140 - RNA, 23 nt, synthetic construct
  misc_feature 1..23: note = Synthetic
  source 1..23: mol_type = other RNA
ttatagagcaagaacactgtttt

SEQ ID NO: 141 - RNA, 23 nt, synthetic construct
  misc_feature 1..23: note = Synthetic
  source 1..23: mol_type = other RNA
ttatagagcaagaacactgtttt

SEQ ID NO: 142 - RNA, 23 nt, synthetic construct
  misc_feature 1..23: note = Synthetic
  source 1..23: mol_type = other RNA
ttatagagcaagaacactgtttt

SEQ ID NO: 143 - RNA, 23 nt, synthetic construct
  misc_feature 1..23: note = Synthetic
  source 1..23: mol_type = other RNA
ttatagagcaagaacactgtttt

SEQ ID NO: 144 - RNA, 23 nt, synthetic construct
  misc_feature 1..23: note = Synthetic
  source 1..23: mol_type = other RNA
ttatagagcaagaacactgtttt

SEQ ID NO: 145 - RNA, 23 nt, synthetic construct
  misc_feature 1..23: note = Synthetic
  source 1..23: mol_type = other RNA
ttatagagcaagaacactgtttt

SEQ ID NO: 146 - RNA, 23 nt, synthetic construct
  misc_feature 1..23: note = Synthetic
  source 1..23: mol_type = other RNA
ttatagagcaagaacactgtttt

SEQ ID NO: 147 - RNA, 23 nt, synthetic construct
  misc_feature 1..23: note = Synthetic
  source 1..23: mol_type = other RNA
ttatagagcaagaacactgtttt

SEQ ID NO: 148 - RNA, 23 nt, synthetic construct
  misc_feature 1..23: note = Synthetic
  source 1..23: mol_type = other RNA
ttatagagcaagaacactgtttt

SEQ ID NO: 149 - RNA, 23 nt, synthetic construct
  misc_feature 1..23: note = Synthetic
  source 1..23: mol_type = other RNA
ttatagagcaagaacactgtttt

SEQ ID NO: 150 - RNA, 23 nt, synthetic construct
  misc_feature 1..23: note = Synthetic
  source 1..23: mol_type = other RNA
ttatagagcaagaacactgtttt

SEQ ID NO: 151 - RNA, 23 nt, synthetic construct
  misc_feature 1..23: note = Synthetic
  source 1..23: mol_type = other RNA
ttatagagcaagaacactgtttt

SEQ ID NO: 152 - RNA, 23 nt, synthetic construct
  misc_feature 1..23: note = Synthetic
  source 1..23: mol_type = other RNA
ttatagagcaagaacactgtttt

SEQ ID NO: 153 - RNA, 23 nt, synthetic construct
  misc_feature 1..23: note = Synthetic
  source 1..23: mol_type = other RNA
ttatagagcaagaacactgtttt

SEQ ID NO: 154 - RNA, 23 nt, synthetic construct
  misc_feature 1..23: note = Synthetic
  source 1..23: mol_type = other RNA
ttatagagcaagaacactgtttt

SEQ ID NO: 155 - RNA, 23 nt, synthetic construct
  misc_feature 1..23: note = Synthetic
  source 1..23: mol_type = other RNA
ttatagagcaagaacactgtttt

SEQ ID NO: 156 - RNA, 23 nt, synthetic construct
  misc_feature 1..23: note = Synthetic
  source 1..23: mol_type = other RNA
ttatagagcaagaacactgtttt

SEQ ID NO: 157 - RNA, 23 nt, synthetic construct
  misc_feature 1..23: note = Synthetic
  source 1..23: mol_type = other RNA
ttatagagcaagaacactgtttt

SEQ ID NO: 158 - RNA, 23 nt, synthetic construct
  misc_feature 1..23: note = Synthetic
  source 1..23: mol_type = other RNA
ttatagagcaagaacactgtttt

SEQ ID NO: 159 - RNA, 23 nt, synthetic construct
  misc_feature 1..23: note = Synthetic
  source 1..23: mol_type = other RNA
ttatagagcaagaacactgtttt

SEQ ID NO: 160 - RNA, 23 nt, synthetic construct
  misc_feature 1..23: note = Synthetic
  source 1..23: mol_type = other RNA
ttatagagcaagaacactgtttt

SEQ ID NO: 161 - RNA, 23 nt, synthetic construct
  misc_feature 1..23: note = Synthetic
  source 1..23: mol_type = other RNA
ttatagagcaagaacactgtttt

SEQ ID NO: 162 - RNA, 23 nt, synthetic construct
  misc_feature 1..23: note = Synthetic
  source 1..23: mol_type = other RNA
ttatagagcaagaacactgtttt

SEQ ID NO: 163 - RNA, 21 nt, synthetic construct
  misc_feature 1..21: note = Synthetic
  source 1..21: mol_type = other RNA
aacagtgttcttgctctataa

SEQ ID NO: 164 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 165 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 166 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 167 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 168 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 169 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 170 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 171 - RNA, 23 nt, synthetic construct
  misc_feature 1..23: note = Synthetic
  source 1..23: mol_type = other RNA
ttatagagcaagaacactgtttt

SEQ ID NO: 172 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 173 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 174 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 175 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 176 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 177 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 178 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 179 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 180 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 181 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 182 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 183 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 184 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 185 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 186 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 187 - RNA, 23 nt, synthetic construct
  misc_feature 1..23: note = Synthetic
  source 1..23: mol_type = other RNA
ttatagagcaagaacactgtttt

SEQ ID NO: 188 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 189 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 190 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 191 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 192 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 193 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 194 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 195 - RNA, 21 nt, synthetic construct
  misc_feature 1..21: note = Synthetic
  source 1..21: mol_type = other RNA
aacagtgttcttgctctataa

SEQ ID NO: 196 - RNA, 23 nt, synthetic construct
  misc_feature 1..23: note = Synthetic
  source 1..23: mol_type = other RNA
ttatagagcaagaacactgtttt

SEQ ID NO: 197 - RNA, 23 nt, synthetic construct
  misc_feature 1..23: note = Synthetic
  source 1..23: mol_type = other RNA
ttatagagcaagaacactgtttt

SEQ ID NO: 198 - RNA, 23 nt, synthetic construct
  misc_feature 1..23: note = Synthetic
  source 1..23: mol_type = other RNA
ttatagagcaagaacactgtttt

SEQ ID NO: 199 - RNA, 23 nt, synthetic construct
  misc_feature 1..23: note = Synthetic
  source 1..23: mol_type = other RNA
ttatagagcaagaacactgtttt

SEQ ID NO: 200 - RNA, 23 nt, synthetic construct
  misc_feature 1..23: note = Synthetic
  source 1..23: mol_type = other RNA
ttatagagcaagaacactgtttt

SEQ ID NO: 201 - RNA, 23 nt, synthetic construct
  misc_feature 1..23: note = Synthetic
  source 1..23: mol_type = other RNA
ttatagagcaagaacactgtttt

SEQ ID NO: 202 - RNA, 23 nt, synthetic construct
  misc_feature 1..23: note = Synthetic
  source 1..23: mol_type = other RNA
ttatagagcaagaacactgtttt

SEQ ID NO: 203 - RNA, 23 nt, synthetic construct
  misc_feature 1..23: note = Synthetic
  source 1..23: mol_type = other RNA
ttatagagcaagaacactgtttt

SEQ ID NO: 204 - RNA, 23 nt, synthetic construct
  misc_feature 1..23: note = Synthetic
  source 1..23: mol_type = other RNA
ttatagagcaagaacactgtttt

SEQ ID NO: 205 - RNA, 23 nt, synthetic construct
  misc_feature 1..23: note = Synthetic
  source 1..23: mol_type = other RNA
ttatagagcaagaacactgtttt

SEQ ID NO: 206 - RNA, 23 nt, synthetic construct
  misc_feature 1..23: note = Synthetic
  source 1..23: mol_type = other RNA
ttatagagcaagaacactgtttt

SEQ ID NO: 207 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 208 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 209 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 210 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 211 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 212 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 213 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 214 - RNA, 23 nt, synthetic construct
  misc_feature 1..23: note = Synthetic
  source 1..23: mol_type = other RNA
ttatagagcaagaacactgtttt

SEQ ID NO: 215 - RNA, 23 nt, synthetic construct
  misc_feature 1..23: note = Synthetic
  source 1..23: mol_type = other RNA
ttatagagcaagaacactgtttt

SEQ ID NO: 216 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 217 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 218 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 219 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 220 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 221 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 222 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 223 - RNA, 23 nt, synthetic construct
  misc_feature 1..23: note = Synthetic
  source 1..23: mol_type = other RNA
ttatagagcaagaacactgtttt

SEQ ID NO: 224 - RNA, 23 nt, synthetic construct
  misc_feature 1..23: note = Synthetic
  source 1..23: mol_type = other RNA
ttatagagcaagaacactgtttt

SEQ ID NO: 225 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 226 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 227 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 228 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 229 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 230 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 231 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 232 - RNA, 23 nt, synthetic construct
  misc_feature 1..23: note = Synthetic
  source 1..23: mol_type = other RNA
ttatagagcaagaacactgtttt

SEQ ID NO: 233 - RNA, 23 nt, synthetic construct
  misc_feature 1..23: note = Synthetic
  source 1..23: mol_type = other RNA
ttatagagcaagaacactgtttt

SEQ ID NO: 234 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 235 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 236 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 237 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 238 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 239 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 240 - RNA, 23 nt, synthetic construct
  modified_base 1: mod_base = OTHER; note = thymine
  misc_feature 1..23: note = Synthetic
  source 1..23: mol_type = other RNA
ttatagagcaagaacactgtttt

SEQ ID NO: 241 - RNA, 23 nt, synthetic construct
  misc_feature 1..23: note = Synthetic
  source 1..23: mol_type = other RNA
ttatagagcaagaacactgtttt

SEQ ID NO: 242 - RNA, 23 nt, synthetic construct
  misc_feature 1..23: note = Synthetic
  source 1..23: mol_type = other RNA
ttatagagcaagaacactgtttt

SEQ ID NO: 243 - RNA, 23 nt, synthetic construct
  misc_feature 1..23: note = Synthetic
  source 1..23: mol_type = other RNA
ttatagagcaagaacactgtttt

SEQ ID NO: 244 - RNA, 23 nt, synthetic construct
  misc_feature 1..23: note = Synthetic
  source 1..23: mol_type = other RNA
ttatagagcaagaacactgtttt

SEQ ID NO: 245 - RNA, 23 nt, synthetic construct
  misc_feature 1..23: note = Synthetic
  source 1..23: mol_type = other RNA
ttatagagcaagaacactgtttt

SEQ ID NO: 246 - RNA, 23 nt, synthetic construct
  misc_feature 1..23: note = Synthetic
  source 1..23: mol_type = other RNA
ttatagagcaagaacactgtttt

SEQ ID NO: 247 - RNA, 23 nt, synthetic construct
  misc_feature 1..23: note = Synthetic
  source 1..23: mol_type = other RNA
ttatagagcaagaacactgtttt

SEQ ID NO: 248 - RNA, 23 nt, synthetic construct
  misc_feature 1..23: note = Synthetic
  source 1..23: mol_type = other RNA
ttatagagcaagaacactgtttt

SEQ ID NO: 249 - RNA, 23 nt, synthetic construct
  misc_feature 1..23: note = Synthetic
  source 1..23: mol_type = other RNA
ttatagagcaagaacactgtttt

SEQ ID NO: 250 - RNA, 23 nt, synthetic construct
  misc_feature 1..23: note = Synthetic
  source 1..23: mol_type = other RNA
aacagtgttcttgctctataatt

SEQ ID NO: 251 - RNA, 23 nt, synthetic construct
  misc_feature 1..23: note = Synthetic
  source 1..23: mol_type = other RNA
aacagtgttcttgctctataatt

SEQ ID NO: 252 - RNA, 23 nt, synthetic construct
  misc_feature 1..23: note = Synthetic
  source 1..23: mol_type = other RNA
ttatagagcaagaacactgtttt

SEQ ID NO: 253 - RNA, 23 nt, synthetic construct
  misc_feature 1..23: note = Synthetic
  source 1..23: mol_type = other RNA
ttatagagcaagaacactgtttt

SEQ ID NO: 254 - RNA, 23 nt, synthetic construct
  misc_feature 1..23: note = Synthetic
  source 1..23: mol_type = other RNA
ttatagagcaagaacactgtttt

SEQ ID NO: 255 - RNA, 23 nt, synthetic construct
  misc_feature 1..23: note = Synthetic
  source 1..23: mol_type = other RNA
ttatagagcaagaacactgtttt

SEQ ID NO: 256 - RNA, 23 nt, synthetic construct
  misc_feature 1..23: note = Synthetic
  source 1..23: mol_type = other RNA
ttatagagcaagaacactgtttt

SEQ ID NO: 257 - RNA, 23 nt, synthetic construct
  misc_feature 1..23: note = Synthetic
  source 1..23: mol_type = other RNA
ttatagagcaagaacactgtttt

SEQ ID NO: 258 - RNA, 23 nt, synthetic construct
  misc_feature 1..23: note = Synthetic
  source 1..23: mol_type = other RNA
ttatagagcaagaacactgtttt

SEQ ID NO: 259 - RNA, 23 nt, synthetic construct
  misc_feature 1..23: note = Synthetic
  source 1..23: mol_type = other RNA
ttatagagcaagaacactgtttt

SEQ ID NO: 260 - RNA, 23 nt, synthetic construct
  misc_feature 1..23: note = Synthetic
  source 1..23: mol_type = other RNA
ttatagagcaagaacactgtttt

SEQ ID NO: 261 - RNA, 23 nt, synthetic construct
  misc_feature 1..23: note = Synthetic
  source 1..23: mol_type = other RNA
ttatagagcaagaacactgtttt

SEQ ID NO: 262 - RNA, 23 nt, synthetic construct
  misc_feature 1..23: note = Synthetic
  source 1..23: mol_type = other RNA
ttatagagcaagaacactgtttt

SEQ ID NO: 263 - RNA, 23 nt, synthetic construct
  misc_feature 1..23: note = Synthetic
  source 1..23: mol_type = other RNA
ttatagagcaagaacactgtttt

SEQ ID NO: 264 - RNA, 23 nt, synthetic construct
  misc_feature 1..23: note = Synthetic
  source 1..23: mol_type = other RNA
ttatagagcaagaacactgtttt

SEQ ID NO: 265 - RNA, 23 nt, synthetic construct
  misc_feature 1..23: note = Synthetic
  source 1..23: mol_type = other RNA
ttatagagcaagaacactgtttt

SEQ ID NO: 266 - RNA, 23 nt, synthetic construct
  misc_feature 1..23: note = Synthetic
  source 1..23: mol_type = other RNA
ttatagagcaagaacactgtttt

SEQ ID NO: 267 - RNA, 23 nt, synthetic construct
  misc_feature 1..23: note = Synthetic
  source 1..23: mol_type = other RNA
ttatagagcaagaacactgtttt

SEQ ID NO: 268 - RNA, 23 nt, synthetic construct
  misc_feature 1..23: note = Synthetic
  source 1..23: mol_type = other RNA
ttatagagcaagaacactgtttt

SEQ ID NO: 269 - RNA, 23 nt, synthetic construct
  misc_feature 1..23: note = Synthetic
  source 1..23: mol_type = other RNA
ttatagagcaagaacactgtttt

SEQ ID NO: 270 - RNA, 23 nt, synthetic construct
  misc_feature 1..23: note = Synthetic
  source 1..23: mol_type = other RNA
ttatagagcaagaacactgtttt

SEQ ID NO: 271 - RNA, 23 nt, synthetic construct
  misc_feature 1..23: note = Synthetic
  source 1..23: mol_type = other RNA
ttatagagcaagaacactgtttt

SEQ ID NO: 272 - RNA, 23 nt, synthetic construct
  misc_feature 1..23: note = Synthetic
  source 1..23: mol_type = other RNA
ttatagagcaagaacactgtttt

SEQ ID NO: 273 - RNA, 23 nt, synthetic construct
  misc_feature 1..23: note = Synthetic
  source 1..23: mol_type = other RNA
ttatagagcaagaacactgtttt

SEQ ID NO: 274 - RNA, 23 nt, synthetic construct
  misc_feature 1..23: note = Synthetic
  source 1..23: mol_type = other RNA
ttatagagcaagaacactgtttt

SEQ ID NO: 275 - RNA, 23 nt, synthetic construct
  misc_feature 1..23: note = Synthetic
  source 1..23: mol_type = other RNA
ttatagagcaagaacactgtttt

SEQ ID NO: 276 - RNA, 23 nt, synthetic construct
  misc_feature 1..23: note = Synthetic
  source 1..23: mol_type = other RNA
ttatagagcaagaacactgtttt

SEQ ID NO: 277 - RNA, 23 nt, synthetic construct
  misc_feature 1..23: note = Synthetic
  source 1..23: mol_type = other RNA
ttatagagcaagaacactgtttt

SEQ ID NO: 278 - RNA, 23 nt, synthetic construct
  misc_feature 1..23: note = Synthetic
  source 1..23: mol_type = other RNA
ttatagagcaagaacactgtttt

SEQ ID NO: 279 - RNA, 23 nt, synthetic construct
  misc_feature 1..23: note = Synthetic
  source 1..23: mol_type = other RNA
ttatagagcaagaacactgtttt

SEQ ID NO: 280 - RNA, 23 nt, synthetic construct
  misc_feature 1..23: note = Synthetic
  source 1..23: mol_type = other RNA
ttatagagcaagaacactgtttt

SEQ ID NO: 281 - RNA, 23 nt, synthetic construct
  misc_feature 1..23: note = Synthetic
  source 1..23: mol_type = other RNA
ttatagagcaagaacactgtttt

SEQ ID NO: 282 - RNA, 23 nt, synthetic construct
  misc_feature 1..23: note = Synthetic
  source 1..23: mol_type = other RNA
ttatagagcaagaacactgtttt

SEQ ID NO: 283 - RNA, 23 nt, synthetic construct
  misc_feature 1..23: note = Synthetic
  source 1..23: mol_type = other RNA
ttatagagcaagaacactgtttt

SEQ ID NO: 284 - RNA, 23 nt, synthetic construct
  misc_feature 1..23: note = Synthetic
  source 1..23: mol_type = other RNA
ttatagagcaagaacactgtttt

SEQ ID NO: 285 - RNA, 23 nt, synthetic construct
  misc_feature 1..23: note = Synthetic
  source 1..23: mol_type = other RNA
ttatagagcaagaacactgtttt

SEQ ID NO: 286 - RNA, 23 nt, synthetic construct
  misc_feature 1..23: note = Synthetic
  source 1..23: mol_type = other RNA
ttatagagcaagaacactgtttt

SEQ ID NO: 287 - RNA, 23 nt, synthetic construct
  misc_feature 1..23: note = Synthetic
  source 1..23: mol_type = other RNA
ttatagagcaagaacactgtttt

SEQ ID NO: 288 - RNA, 23 nt, synthetic construct
  misc_feature 1..23: note = Synthetic
  source 1..23: mol_type = other RNA
ttatagagcaagaacactgtttt

SEQ ID NO: 289 - RNA, 23 nt, synthetic construct
  misc_feature 1..23: note = Synthetic
  source 1..23: mol_type = other RNA
ttatagagcaagaacactgtttt

SEQ ID NO: 290 - RNA, 23 nt, synthetic construct
  misc_feature 1..23: note = Synthetic
  source 1..23: mol_type = other RNA
ttatagagcaagaacactgtttt

SEQ ID NO: 291 - RNA, 23 nt, synthetic construct
  misc_feature 1..23: note = Synthetic
  source 1..23: mol_type = other RNA
ttatagagcaagaacactgtttt

SEQ ID NO: 292 - RNA, 23 nt, synthetic construct
  misc_feature 1..23: note = Synthetic
  source 1..23: mol_type = other RNA
ttatagagcaagaacactgtttt

SEQ ID NO: 293 - RNA, 23 nt, synthetic construct
  misc_feature 1..23: note = Synthetic
  source 1..23: mol_type = other RNA
ttatagagcaagaacactgtttt

SEQ ID NO: 294 - RNA, 23 nt, synthetic construct
  misc_feature 1..23: note = Synthetic
  source 1..23: mol_type = other RNA
ttatagagcaagaacactgtttt

SEQ ID NO: 295 - RNA, 23 nt, synthetic construct
  misc_feature 1..23: note = Synthetic
  source 1..23: mol_type = other RNA
ttatagagcaagaacactgtttt

SEQ ID NO: 296 - RNA, 23 nt, synthetic construct
  misc_feature 1..23: note = Synthetic
  source 1..23: mol_type = other RNA
ttatagagcaagaacactgtttt

SEQ ID NO: 297 - RNA, 23 nt, synthetic construct
  misc_feature 1..23: note = Synthetic
  source 1..23: mol_type = other RNA
ttatagagcaagaacactgtttt

SEQ ID NO: 298 - RNA, 23 nt, synthetic construct
  misc_feature 1..23: note = Synthetic
  source 1..23: mol_type = other RNA
ttatagagcaagaacactgtttt

SEQ ID NO: 299 - RNA, 23 nt, synthetic construct
  misc_feature 1..23: note = Synthetic
  source 1..23: mol_type = other RNA
ttatagagcaagaacactgtttt

SEQ ID NO: 300 - RNA, 23 nt, synthetic construct
  misc_feature 1..23: note = Synthetic
  source 1..23: mol_type = other RNA
ttatagagcaagaacactgtttt

SEQ ID NO: 301 - RNA, 23 nt, synthetic construct
  misc_feature 1..23: note = Synthetic
  source 1..23: mol_type = other RNA
ttatagagcaagaacactgtttt

SEQ ID NO: 302 - RNA, 23 nt, synthetic construct
  misc_feature 1..23: note = Synthetic
  source 1..23: mol_type = other RNA
ttatagagcaagaacactgtttt

SEQ ID NO: 303 - RNA, 23 nt, synthetic construct
  misc_feature 1..23: note = Synthetic
  source 1..23: mol_type = other RNA
ttatagagcaagaacactgtttt

SEQ ID NO: 304 - RNA, 23 nt, synthetic construct
  misc_feature 1..23: note = Synthetic
  source 1..23: mol_type = other RNA
ttatagagcaagaacactgtttt

SEQ ID NO: 305 - RNA, 23 nt, synthetic construct
  misc_feature 1..23: note = Synthetic
  source 1..23: mol_type = other RNA
ttatagagcaagaacactgtttt

SEQ ID NO: 306 - RNA, 23 nt, synthetic construct
  misc_feature 1..23: note = Synthetic
  source 1..23: mol_type = other RNA
ttatagagcaagaacactgtttt

SEQ ID NO: 307 - RNA, 23 nt, synthetic construct
  misc_feature 1..23: note = Synthetic
  source 1..23: mol_type = other RNA
ttatagagcaagaacactgtttt

SEQ ID NO: 308 - RNA, 23 nt, synthetic construct
  misc_feature 1..23: note = Synthetic
  source 1..23: mol_type = other RNA
ttatagagcaagaacactgtttt

SEQ ID NO: 309 - RNA, 23 nt, synthetic construct
  misc_feature 1..23: note = Synthetic
  source 1..23: mol_type = other RNA
ttatagagcaagaacactgtttt

SEQ ID NO: 310 - RNA, 23 nt, synthetic construct
  misc_feature 1..23: note = Synthetic
  source 1..23: mol_type = other RNA
ttatagagcaagaacactgtttt

SEQ ID NO: 311 - RNA, 23 nt, synthetic construct
  misc_feature 1..23: note = Synthetic
  source 1..23: mol_type = other RNA
ttatagagcaagaacactgtttt

SEQ ID NO: 312 - RNA, 23 nt, synthetic construct
  misc_feature 1..23: note = Synthetic
  source 1..23: mol_type = other RNA
ttatagagcaagaacactgtttt

SEQ ID NO: 313 - RNA, 23 nt, synthetic construct
  misc_feature 1..23: note = Synthetic
  source 1..23: mol_type = other RNA
ttatagagcaagaacactgtttt

SEQ ID NO: 314 - RNA, 23 nt, synthetic construct
  misc_feature 1..23: note = Synthetic
  source 1..23: mol_type = other RNA
ttatagagcaagaacactgtttt

SEQ ID NO: 315 - RNA, 23 nt, synthetic construct
  misc_feature 1..23: note = Synthetic
  source 1..23: mol_type = other RNA
ttatagagcaagaacactgtttt

SEQ ID NO: 316 - RNA, 23 nt, synthetic construct
  misc_feature 1..23: note = Synthetic
  source 1..23: mol_type = other RNA
ttatagagcaagaacactgtttt

SEQ ID NO: 317 - RNA, 23 nt, synthetic construct
  misc_feature 1..23: note = Synthetic
  source 1..23: mol_type = other RNA
ttatagagcaagaacactgtttt

SEQ ID NO: 318 - RNA, 23 nt, synthetic construct
  misc_feature 1..23: note = Synthetic
  source 1..23: mol_type = other RNA
ttatagagcaagaacactgtttt

SEQ ID NO: 319 - RNA, 23 nt, synthetic construct
  misc_feature 1..23: note = Synthetic
  source 1..23: mol_type = other RNA
ttatagagcaagaacactgtttt

SEQ ID NO: 320 - RNA, 23 nt, synthetic construct
  misc_feature 1..23: note = Synthetic
  source 1..23: mol_type = other RNA
ttatagagcaagaacactgtttt

SEQ ID NO: 321 - RNA, 23 nt, synthetic construct
  misc_feature 1..23: note = Synthetic
  source 1..23: mol_type = other RNA
ttatagagcaagaacactgtttt

SEQ ID NO: 322 - RNA, 23 nt, synthetic construct
  misc_feature 1..23: note = Synthetic
  source 1..23: mol_type = other RNA
ttatagagcaagaacactgtttt

SEQ ID NO: 323 - RNA, 23 nt, synthetic construct
  misc_feature 1..23: note = Synthetic
  source 1..23: mol_type = other RNA
ttatagagcaagaacactgtttt

SEQ ID NO: 324 - RNA, 23 nt, synthetic construct
  misc_feature 1..23: note = Synthetic
  source 1..23: mol_type = other RNA
ttatagagcaagaacactgtttt

SEQ ID NO: 325 - RNA, 23 nt, synthetic construct
  misc_feature 1..23: note = Synthetic
  source 1..23: mol_type = other RNA
ttatagagcaagaacactgtttt

SEQ ID NO: 326 - RNA, 23 nt, synthetic construct
  misc_feature 1..23: note = Synthetic
  source 1..23: mol_type = other RNA
ttatagagcaagaacactgtttt

SEQ ID NO: 327 - RNA, 23 nt, synthetic construct
  misc_feature 1..23: note = Synthetic
  source 1..23: mol_type = other RNA
ttatagagcaagaacactgtttt

SEQ ID NO: 328 - RNA, 23 nt, synthetic construct
  misc_feature 1..23: note = Synthetic
  source 1..23: mol_type = other RNA
ttatagagcaagaacactgtttt

SEQ ID NO: 329 - RNA, 23 nt, synthetic construct
  misc_feature 1..23: note = Synthetic
  source 1..23: mol_type = other RNA
ttatagagcaagaacactgtttt

SEQ ID NO: 330 - RNA, 23 nt, synthetic construct
  misc_feature 1..23: note = Synthetic
  source 1..23: mol_type = other RNA
ttatagagcaagaacactgtttt

SEQ ID NO: 331 - RNA, 23 nt, synthetic construct
  misc_feature 1..23: note = Synthetic
  source 1..23: mol_type = other RNA
ttatagagcaagaacactgtttt

SEQ ID NO: 332 - RNA, 23 nt, synthetic construct
  misc_feature 1..23: note = Synthetic
  source 1..23: mol_type = other RNA
ttatagagcaagaacactgtttt

SEQ ID NO: 333 - RNA, 23 nt, synthetic construct
  misc_feature 1..23: note = Synthetic
  source 1..23: mol_type = other RNA
ttatagagcaagaacactgtttt

SEQ ID NO: 334 - RNA, 23 nt, synthetic construct
  misc_feature 1..23: note = Synthetic
  source 1..23: mol_type = other RNA
ttatagagcaagaacactgtttt

SEQ ID NO: 335 - RNA, 23 nt, synthetic construct
  misc_feature 1..23: note = Synthetic
  source 1..23: mol_type = other RNA
ttatagagcaagaacactgtttt

SEQ ID NO: 336 - RNA, 23 nt, synthetic construct
  misc_feature 1..23: note = Synthetic
  source 1..23: mol_type = other RNA
ttatagagcaagaacactgtttt

SEQ ID NO: 337 - RNA, 23 nt, synthetic construct
  misc_feature 1..23: note = Synthetic
  source 1..23: mol_type = other RNA
ttatagagcaagaacactgtttt

SEQ ID NO: 338 - RNA, 23 nt, synthetic construct
  misc_feature 1..23: note = Synthetic
  source 1..23: mol_type = other RNA
ttatagagcaagaacactgtttt

SEQ ID NO: 339 - RNA, 23 nt, synthetic construct
  misc_feature 1..23: note = Synthetic
  source 1..23: mol_type = other RNA
ttatagagcaagaacactgtttt

SEQ ID NO: 340 - RNA, 23 nt, synthetic construct
  misc_feature 1..23: note = Synthetic
  source 1..23: mol_type = other RNA
ttatagagcaagaacactgtttt

SEQ ID NO: 341 - RNA, 23 nt, synthetic construct
  misc_feature 1..23: note = Synthetic
  source 1..23: mol_type = other RNA
ttatagagcaagaacactgtttt

SEQ ID NO: 342 - RNA, 23 nt, synthetic construct
  misc_feature 1..23: note = Synthetic
  source 1..23: mol_type = other RNA
ttatagagcaagaacactgtttt

SEQ ID NO: 343 - RNA, 23 nt, synthetic construct
  misc_feature 1..23: note = Synthetic
  source 1..23: mol_type = other RNA
ttatagagcaagaacactgtttt

SEQ ID NO: 344 - RNA, 23 nt, synthetic construct
  misc_feature 1..23: note = Synthetic
  source 1..23: mol_type = other RNA
ttatagagcaagaacactgtttt

SEQ ID NO: 345 - RNA, 23 nt, synthetic construct
  misc_feature 1..23: note = Synthetic
  source 1..23: mol_type = other RNA
ttatagagcaagaacactgtttt

SEQ ID NO: 346 - RNA, 23 nt, synthetic construct
  misc_feature 1..23: note = Synthetic
  source 1..23: mol_type = other RNA
ttatagagcaagaacactgtttt

SEQ ID NO: 347 - RNA, 23 nt, synthetic construct
  misc_feature 1..23: note = Synthetic
  source 1..23: mol_type = other RNA
ttatagagcaagaacactgtttt

SEQ ID NO: 348 - RNA, 23 nt, synthetic construct
  misc_feature 1..23: note = Synthetic
  source 1..23: mol_type = other RNA
ttatagagcaagaacactgtttt

SEQ ID NO: 349 - RNA, 23 nt, synthetic construct
  misc_feature 1..23: note = Synthetic
  source 1..23: mol_type = other RNA
ttatagagcaagaacactgtttt

SEQ ID NO: 350 - RNA, 23 nt, synthetic construct
  misc_feature 1..23: note = Synthetic
  source 1..23: mol_type = other RNA
ttatagagcaagaacactgtttt

SEQ ID NO: 351 - RNA, 23 nt, synthetic construct
  misc_feature 1..23: note = Synthetic
  source 1..23: mol_type = other RNA
ttatagagcaagaacactgtttt

SEQ ID NO: 352 - RNA, 23 nt, synthetic construct
  misc_feature 1..23: note = Synthetic
  source 1..23: mol_type = other RNA
ttatagagcaagaacactgtttt

SEQ ID NO: 353 - RNA, 23 nt, synthetic construct
  misc_feature 1..23: note = Synthetic
  source 1..23: mol_type = other RNA
ttatagagcaagaacactgtttt

SEQ ID NO: 354 - RNA, 23 nt, synthetic construct
  misc_feature 1..23: note = Synthetic
  source 1..23: mol_type = other RNA
ttatagagcaagaacactgtttt

SEQ ID NO: 355 - RNA, 23 nt, synthetic construct
  misc_feature 1..23: note = Synthetic
  source 1..23: mol_type = other RNA
ttatagagcaagaacactgtttt

SEQ ID NO: 356 - RNA, 23 nt, synthetic construct
  misc_feature 1..23: note = Synthetic
  source 1..23: mol_type = other RNA
ttatagagcaagaacactgtttt

SEQ ID NO: 357 - RNA, 23 nt, synthetic construct
  misc_feature 1..23: note = Synthetic
  source 1..23: mol_type = other RNA
ttatagagcaagaacactgtttt

SEQ ID NO: 358 - RNA, 23 nt, synthetic construct
  misc_feature 1..23: note = Synthetic
  source 1..23: mol_type = other RNA
ttatagagcaagaacactgtttt

SEQ ID NO: 359 - RNA, 23 nt, synthetic construct
  misc_feature 1..23: note = Synthetic
  source 1..23: mol_type = other RNA
ttatagagcaagaacactgtttt

SEQ ID NO: 360 - RNA, 23 nt, synthetic construct
  misc_feature 1..23: note = Synthetic
  source 1..23: mol_type = other RNA
ttatagagcaagaacactgtttt

SEQ ID NO: 361 - RNA, 23 nt, synthetic construct
  misc_feature 1..23: note = Synthetic
  source 1..23: mol_type = other RNA
ttatagagcaagaacactgtttt

SEQ ID NO: 362 - RNA, 23 nt, synthetic construct
  misc_feature 1..23: note = Synthetic
  source 1..23: mol_type = other RNA
ttatagagcaagaacactgtttt

SEQ ID NO: 363 - RNA, 23 nt, synthetic construct
  misc_feature 1..23: note = Synthetic
  source 1..23: mol_type = other RNA
ttatagagcaagaacactgtttt

SEQ ID NO: 364 - RNA, 23 nt, synthetic construct
  misc_feature 1..23: note = Synthetic
  source 1..23: mol_type = other RNA
ttatagagcaagaacactgtttt

SEQ ID NO: 365 - RNA, 23 nt, synthetic construct
  misc_feature 1..23: note = Synthetic
  source 1..23: mol_type = other RNA
ttatagagcaagaacactgtttt

SEQ ID NO: 366 - RNA, 23 nt, synthetic construct
  misc_feature 1..23: note = Synthetic
  source 1..23: mol_type = other RNA
ttatagagcaagaacactgtttt

SEQ ID NO: 367 - RNA, 23 nt, synthetic construct
  misc_feature 1..23: note = Synthetic
  source 1..23: mol_type = other RNA
ttatagagcaagaacactgtttt

SEQ ID NO: 368 - RNA, 23 nt, synthetic construct
  misc_feature 1..23: note = Synthetic
  source 1..23: mol_type = other RNA
ttatagagcaagaacactgtttt

SEQ ID NO: 369 - RNA, 23 nt, synthetic construct
  misc_feature 1..23: note = Synthetic
  source 1..23: mol_type = other RNA
ttatagagcaagaacactgtttt

SEQ ID NO: 370 - RNA, 23 nt, synthetic construct
  misc_feature 1..23: note = Synthetic
  source 1..23: mol_type = other RNA
ttatagagcaagaacactgtttt

SEQ ID NO: 371 - RNA, 23 nt, synthetic construct
  misc_feature 1..23: note = Synthetic
  source 1..23: mol_type = other RNA
ttatagagcaagaacactgtttt

SEQ ID NO: 372 - RNA, 23 nt, synthetic construct
  misc_feature 1..23: note = Synthetic
  source 1..23: mol_type = other RNA
ttatagagcaagaacactgtttt

SEQ ID NO: 373 - RNA, 23 nt, synthetic construct
  misc_feature 1..23: note = Synthetic
  source 1..23: mol_type = other RNA
ttatagagcaagaacactgtttt

SEQ ID NO: 374 - RNA, 23 nt, synthetic construct
  misc_feature 1..23: note = Synthetic
  source 1..23: mol_type = other RNA
ttatagagcaagaacactgtttt

SEQ ID NO: 375 - RNA, 23 nt, synthetic construct
  misc_feature 1..23: note = Synthetic
  source 1..23: mol_type = other RNA
ttatagagcaagaacactgtttt

SEQ ID NO: 376 - RNA, 23 nt, synthetic construct
  misc_feature 1..23: note = Synthetic
  source 1..23: mol_type = other RNA
ttatagagcaagaacactgtttt

SEQ ID NO: 377 - RNA, 23 nt, synthetic construct
  misc_feature 1..23: note = Synthetic
  source 1..23: mol_type = other RNA
ttatagagcaagaacactgtttt

SEQ ID NO: 378 - RNA, 23 nt, synthetic construct
  misc_feature 1..23: note = Synthetic
  source 1..23: mol_type = other RNA
ttatagagcaagaacactgtttt

SEQ ID NO: 379 - RNA, 23 nt, synthetic construct
  misc_feature 1..23: note = Synthetic
  source 1..23: mol_type = other RNA
ttatagagcaagaacactgtttt

SEQ ID NO: 380 - RNA, 23 nt, synthetic construct
  misc_feature 1..23: note = Synthetic
  source 1..23: mol_type = other RNA
ttatagagcaagaacactgtttt

SEQ ID NO: 381 - RNA, 23 nt, synthetic construct
  misc_feature 1..23: note = Synthetic
  source 1..23: mol_type = other RNA
ttatagagcaagaacactgtttt

SEQ ID NO: 382 - RNA, 23 nt, synthetic construct
  misc_feature 1..23: note = Synthetic
  source 1..23: mol_type = other RNA
ttatagagcaagaacactgtttt

SEQ ID NO: 383 - RNA, 23 nt, synthetic construct
  misc_feature 1..23: note = Synthetic
  source 1..23: mol_type = other RNA
ttatagagcaagaacactgtttt

SEQ ID NO: 384 - RNA, 23 nt, synthetic construct
  misc_feature 1..23: note = Synthetic
  source 1..23: mol_type = other RNA
ttatagagcaagaacactgtttt

SEQ ID NO: 385 - RNA, 23 nt, synthetic construct
  misc_feature 1..23: note = Synthetic
  source 1..23: mol_type = other RNA
ttatagagcaagaacactgtttt

SEQ ID NO: 386 - RNA, 23 nt, synthetic construct
  misc_feature 1..23: note = Synthetic
  source 1..23: mol_type = other RNA
ttatagagcaagaacactgtttt

SEQ ID NO: 387 - RNA, 23 nt, synthetic construct
  misc_feature 1..23: note = Synthetic
  source 1..23: mol_type = other RNA
ttatagagcaagaacactgtttt

SEQ ID NO: 388 - RNA, 23 nt, synthetic construct
  misc_feature 1..23: note = Synthetic
  source 1..23: mol_type = other RNA
ttatagagcaagaacactgtttt

SEQ ID NO: 389 - RNA, 23 nt, synthetic construct
  misc_feature 1..23: note = Synthetic
  source 1..23: mol_type = other RNA
ttatagagcaagaacactgtttt

SEQ ID NO: 390 - RNA, 23 nt, synthetic construct
  misc_feature 1..23: note = Synthetic
  source 1..23: mol_type = other RNA
ttatagagcaagaacactgtttt

SEQ ID NO: 391 - RNA, 23 nt, synthetic construct
  misc_feature 1..23: note = Synthetic
  source 1..23: mol_type = other RNA
ttatagagcaagaacactgtttt

SEQ ID NO: 392 - RNA, 23 nt, synthetic construct
  misc_feature 1..23: note = Synthetic
  source 1..23: mol_type = other RNA
ttatagagcaagaacactgtttt

SEQ ID NO: 393 - RNA, 23 nt, synthetic construct
  misc_feature 1..23: note = Synthetic
  source 1..23: mol_type = other RNA
ttatagagcaagaacactgtttt

SEQ ID NO: 394 - RNA, 23 nt, synthetic construct
  misc_feature 1..23: note = Synthetic
  source 1..23: mol_type = other RNA
ttatagagcaagaacactgtttt

SEQ ID NO: 395 - RNA, 23 nt, synthetic construct
  misc_feature 1..23: note = Synthetic
  source 1..23: mol_type = other RNA
ttatagagcaagaacactgtttt

SEQ ID NO: 396 - RNA, 23 nt, synthetic construct
  misc_feature 1..23: note = Synthetic
  source 1..23: mol_type = other RNA
ttatagagcaagaacactgtttt

SEQ ID NO: 397 - RNA, 23 nt, synthetic construct
  misc_feature 1..23: note = Synthetic
  source 1..23: mol_type = other RNA
ttatagagcaagaacactgtttt

SEQ ID NO: 398 - RNA, 23 nt, synthetic construct
  misc_feature 1..23: note = Synthetic
  source 1..23: mol_type = other RNA
ttatagagcaagaacactgtttt

SEQ ID NO: 399 - RNA, 23 nt, synthetic construct
  misc_feature 1..23: note = Synthetic
  source 1..23: mol_type = other RNA
ttatagagcaagaacactgtttt

SEQ ID NO: 400 - RNA, 23 nt, synthetic construct
  misc_feature 1..23: note = Synthetic
  source 1..23: mol_type = other RNA
ttatagagcaagaacactgtttt

SEQ ID NO: 401 - RNA, 23 nt, synthetic construct
  misc_feature 1..23: note = Synthetic
  source 1..23: mol_type = other RNA
ttatagagcaagaacactgtttt

SEQ ID NO: 402 - RNA, 23 nt, synthetic construct
  misc_feature 1..23: note = Synthetic
  source 1..23: mol_type = other RNA
ttatagagcaagaacactgtttt

SEQ ID NO: 403 - RNA, 23 nt, synthetic construct
  misc_feature 1..23: note = Synthetic
  source 1..23: mol_type = other RNA
ttatagagcaagaacactgtttt

SEQ ID NO: 404 - RNA, 23 nt, synthetic construct
  misc_feature 1..23: note = Synthetic
  source 1..23: mol_type = other RNA
ttatagagcaagaacactgtttt

SEQ ID NO: 405 - RNA, 23 nt, synthetic construct
  misc_feature 1..23: note = Synthetic
  source 1..23: mol_type = other RNA
ttatagagcaagaacactgtttt

SEQ ID NO: 406 - RNA, 23 nt, synthetic construct
  misc_feature 1..23: note = Synthetic
  source 1..23: mol_type = other RNA
ttatagagcaagaacactgtttt

SEQ ID NO: 407 - RNA, 23 nt, synthetic construct
  misc_feature 1..23: note = Synthetic
  source 1..23: mol_type = other RNA
ttatagagcaagaacactgtttt

SEQ ID NO: 408 - RNA, 23 nt, synthetic construct
  misc_feature 1..23: note = Synthetic
  source 1..23: mol_type = other RNA
ttatagagcaagaacactgtttt

SEQ ID NO: 409 - RNA, 23 nt, synthetic construct
  misc_feature 1..23: note = Synthetic
  source 1..23: mol_type = other RNA
ttatagagcaagaacactgtttt

SEQ ID NO: 410 - RNA, 23 nt, synthetic construct
  misc_feature 1..23: note = Synthetic
  source 1..23: mol_type = other RNA
ttatagagcaagaacactgtttt

SEQ ID NO: 411 - RNA, 23 nt, synthetic construct
  misc_feature 1..23: note = Synthetic
  source 1..23: mol_type = other RNA
ttatagagcaagaacactgtttt

SEQ ID NO: 412 - RNA, 23 nt, synthetic construct
  misc_feature 1..23: note = Synthetic
  source 1..23: mol_type = other RNA
ttatagagcaagaacactgtttt

SEQ ID NO: 413 - RNA, 23 nt, synthetic construct
  misc_feature 1..23: note = Synthetic
  source 1..23: mol_type = other RNA
ttatagagcaagaacactgtttt

SEQ ID NO: 414 - RNA, 23 nt, synthetic construct
  misc_feature 1..23: note = Synthetic
  source 1..23: mol_type = other RNA
ttatagagcaagaacactgtttt

SEQ ID NO: 415 - RNA, 23 nt, synthetic construct
  misc_feature 1..23: note = Synthetic
  source 1..23: mol_type = other RNA
ttatagagcaagaacactgtttt

SEQ ID NO: 416 - RNA, 23 nt, synthetic construct
  misc_feature 1..23: note = Synthetic
  source 1..23: mol_type = other RNA
ttatagagcaagaacactgtttt

SEQ ID NO: 417 - RNA, 23 nt, synthetic construct
  misc_feature 1..23: note = Synthetic
  source 1..23: mol_type = other RNA
ttatagagcaagaacactgtttt

SEQ ID NO: 418 - RNA, 23 nt, synthetic construct
  misc_feature 1..23: note = Synthetic
  source 1..23: mol_type = other RNA
ttatagagcaagaacactgtttt

SEQ ID NO: 419 - RNA, 23 nt, synthetic construct
  misc_feature 1..23: note = Synthetic
  source 1..23: mol_type = other RNA
ttatagagcaagaacactgtttt

SEQ ID NO: 420 - RNA, 23 nt, synthetic construct
  misc_feature 1..23: note = Synthetic
  source 1..23: mol_type = other RNA
ttatagagcaagaacactgtttt

SEQ ID NO: 421 - RNA, 23 nt, synthetic construct
  misc_feature 1..23: note = Synthetic
  source 1..23: mol_type = other RNA
ttatagagcaagaacactgtttt

SEQ ID NO: 422 - RNA, 23 nt, synthetic construct
  misc_feature 1..23: note = Synthetic
  source 1..23: mol_type = other RNA
ttatagagcaagaacactgtttt

SEQ ID NO: 423 - RNA, 23 nt, synthetic construct
  misc_feature 1..23: note = Synthetic
  source 1..23: mol_type = other RNA
ttatagagcaagaacactgtttt

SEQ ID NO: 424 - RNA, 23 nt, synthetic construct
  misc_feature 1..23: note = Synthetic
  source 1..23: mol_type = other RNA
ttatagagcaagaacactgtttt

SEQ ID NO: 425 - RNA, 23 nt, synthetic construct
  misc_feature 1..23: note = Synthetic
  source 1..23: mol_type = other RNA
ttatagagcaagaacactgtttt

SEQ ID NO: 426 - RNA, 23 nt, synthetic construct
  misc_feature 1..23: note = Synthetic
  source 1..23: mol_type = other RNA
ttatagagcaagaacactgtttt

SEQ ID NO: 427 - RNA, 23 nt, synthetic construct
  misc_feature 1..23: note = Synthetic
  source 1..23: mol_type = other RNA
ttatagagcaagaacactgtttt

SEQ ID NO: 428 - RNA, 23 nt, synthetic construct
  misc_feature 1..23: note = Synthetic
  source 1..23: mol_type = other RNA
ttatagagcaagaacactgtttt

SEQ ID NO: 429 - RNA, 23 nt, synthetic construct
  misc_feature 1..23: note = Synthetic
  source 1..23: mol_type = other RNA
ttatagagcaagaacactgtttt

SEQ ID NO: 430 - RNA, 23 nt, synthetic construct
  misc_feature 1..23: note = Synthetic
  source 1..23: mol_type = other RNA
ttatagagcaagaacactgtttt

SEQ ID NO: 431 - RNA, 23 nt, synthetic construct
  misc_feature 1..23: note = Synthetic
  source 1..23: mol_type = other RNA
ttatagagcaagaacactgtttt

SEQ ID NO: 432 - RNA, 23 nt, synthetic construct
  misc_feature 1..23: note = Synthetic
  source 1..23: mol_type = other RNA
ttatagagcaagaacactgtttt

SEQ ID NO: 433 - RNA, 23 nt, synthetic construct
  misc_feature 1..23: note = Synthetic
  source 1..23: mol_type = other RNA
ttatagagcaagaacactgtttt

SEQ ID NO: 434 - RNA, 23 nt, synthetic construct
  misc_feature 1..23: note = Synthetic
  source 1..23: mol_type = other RNA
ttatagagcaagaacactgtttt

SEQ ID NO: 435 - RNA, 23 nt, synthetic construct
  misc_feature 1..23: note = Synthetic
  source 1..23: mol_type = other RNA
ttatagagcaagaacactgtttt

SEQ ID NO: 436 - RNA, 23 nt, synthetic construct
  misc_feature 1..23: note = Synthetic
  source 1..23: mol_type = other RNA
ttatagagcaagaacactgtttt

SEQ ID NO: 437 - RNA, 23 nt, synthetic construct
  misc_feature 1..23: note = Synthetic
  source 1..23: mol_type = other RNA
ttatagagcaagaacactgtttt

SEQ ID NO: 438 - RNA, 23 nt, synthetic construct
  misc_feature 1..23: note = Synthetic
  source 1..23: mol_type = other RNA
ttatagagcaagaacactgtttt

SEQ ID NO: 439 - RNA, 23 nt, synthetic construct
  misc_feature 1..23: note = Synthetic
  source 1..23: mol_type = other RNA
ttatagagcaagaacactgtttt

SEQ ID NO: 440 - RNA, 23 nt, synthetic construct
  misc_feature 1..23: note = Synthetic
  source 1..23: mol_type = other RNA
ttatagagcaagaacactgtttt

SEQ ID NO: 441 - RNA, 23 nt, synthetic construct
  misc_feature 1..23: note = Synthetic
  source 1..23: mol_type = other RNA
ttatagagcaagaacactgtttt

SEQ ID NO: 442 - RNA, 23 nt, synthetic construct
  misc_feature 1..23: note = Synthetic
  source 1..23: mol_type = other RNA
ttatagagcaagaacactgtttt

SEQ ID NO: 443 - RNA, 23 nt, synthetic construct
  misc_feature 1..23: note = Synthetic
  source 1..23: mol_type = other RNA
ttatagagcaagaacactgtttt

SEQ ID NO: 444 - RNA, 23 nt, synthetic construct
  misc_feature 1..23: note = Synthetic
  source 1..23: mol_type = other RNA
ttatagagcaagaacactgtttt

SEQ ID NO: 445 - RNA, 23 nt, synthetic construct
  misc_feature 1..23: note = Synthetic
  source 1..23: mol_type = other RNA
ttatagagcaagaacactgtttt

SEQ ID NO: 446 - RNA, 23 nt, synthetic construct
  misc_feature 1..23: note = Synthetic
  source 1..23: mol_type = other RNA
ttatagagcaagaacactgtttt

SEQ ID NO: 447 - RNA, 23 nt, synthetic construct
  misc_feature 1..23: note = Synthetic
  source 1..23: mol_type = other RNA
ttatagagcaagaacactgtttt

SEQ ID NO: 448 - RNA, 23 nt, synthetic construct
  misc_feature 1..23: note = Synthetic
  source 1..23: mol_type = other RNA
ttatagagcaagaacactgtttt

SEQ ID NO: 449 - RNA, 23 nt, synthetic construct
  misc_feature 1..23: note = Synthetic
  source 1..23: mol_type = other RNA
ttatagagcaagaacactgtttt

SEQ ID NO: 450 - RNA, 23 nt, synthetic construct
  misc_feature 1..23: note = Synthetic
  source 1..23: mol_type = other RNA
ttatagagcaagaacactgtttt

SEQ ID NO: 451 - RNA, 23 nt, synthetic construct
  misc_feature 1..23: note = Synthetic
  source 1..23: mol_type = other RNA
ttatagagcaagaacactgtttt

SEQ ID NO: 452 - RNA, 23 nt, synthetic construct
  misc_feature 1..23: note = Synthetic
  source 1..23: mol_type = other RNA
ttatagagcaagaacactgtttt

SEQ ID NO: 453 - RNA, 23 nt, synthetic construct
  misc_feature 1..23: note = Synthetic
  source 1..23: mol_type = other RNA
ttatagagcaagaacactgtttt

SEQ ID NO: 454 - RNA, 23 nt, synthetic construct
  misc_feature 1..23: note = Synthetic
  source 1..23: mol_type = other RNA
ttatagagcaagaacactgtttt

SEQ ID NO: 455 - RNA, 23 nt, synthetic construct
  misc_feature 1..23: note = Synthetic
  source 1..23: mol_type = other RNA
ttatagagcaagaacactgtttt

SEQ ID NO: 456 - RNA, 23 nt, synthetic construct
  misc_feature 1..23: note = Synthetic
  source 1..23: mol_type = other RNA
ttatagagcaagaacactgtttt

SEQ ID NO: 457 - RNA, 23 nt, synthetic construct
  misc_feature 1..23: note = Synthetic
  source 1..23: mol_type = other RNA
ttatagagcaagaacactgtttt

SEQ ID NO: 458 - RNA, 23 nt, synthetic construct
  misc_feature 1..23: note = Synthetic
  source 1..23: mol_type = other RNA
ttatagagcaagaacactgtttt

SEQ ID NO: 459 - RNA, 23 nt, synthetic construct
  misc_feature 1..23: note = Synthetic
  source 1..23: mol_type = other RNA
ttatagagcaagaacactgtttt

SEQ ID NO: 460 - RNA, 23 nt, synthetic construct
  misc_feature 1..23: note = Synthetic
  source 1..23: mol_type = other RNA
ttatagagcaagaacactgtttt

SEQ ID NO: 461 - RNA, 23 nt, synthetic construct
  misc_feature 1..23: note = Synthetic
  source 1..23: mol_type = other RNA
ttatagagcaagaacactgtttt

SEQ ID NO: 462 - RNA, 23 nt, synthetic construct
  misc_feature 1..23: note = Synthetic
  source 1..23: mol_type = other RNA
ttatagagcaagaacactgtttt

SEQ ID NO: 463 - RNA, 23 nt, synthetic construct
  misc_feature 1..23: note = Synthetic
  source 1..23: mol_type = other RNA
ttatagagcaagaacactgtttt

SEQ ID NO: 464 - RNA, 23 nt, synthetic construct
  misc_feature 1..23: note = Synthetic
  source 1..23: mol_type = other RNA
ttatagagcaagaacactgtttt

SEQ ID NO: 465 - RNA, 23 nt, synthetic construct
  misc_feature 1..23: note = Synthetic
  source 1..23: mol_type = other RNA
ttatagagcaagaacactgtttt

SEQ ID NO: 466 - RNA, 23 nt, synthetic construct
  misc_feature 1..23: note = Synthetic
  source 1..23: mol_type = other RNA
ttatagagcaagaacactgtttt

SEQ ID NO: 467 - RNA, 23 nt, synthetic construct
  misc_feature 1..23: note = Synthetic
  source 1..23: mol_type = other RNA
ttatagagcaagaacactgtttt

SEQ ID NO: 468 - RNA, 23 nt, synthetic construct
  misc_feature 1..23: note = Synthetic
  source 1..23: mol_type = other RNA
ttatagagcaagaacactgtttt

SEQ ID NO: 469 - RNA, 23 nt, synthetic construct
  misc_feature 1..23: note = Synthetic
  source 1..23: mol_type = other RNA
ttatagagcaagaacactgtttt

SEQ ID NO: 470 - RNA, 23 nt, synthetic construct
  misc_feature 1..23: note = Synthetic
  source 1..23: mol_type = other RNA
ttatagagcaagaacactgtttt

SEQ ID NO: 471 - RNA, 23 nt, synthetic construct
  misc_feature 1..23: note = Synthetic
  source 1..23: mol_type = other RNA
ttatagagcaagaacactgtttt

SEQ ID NO: 472 - RNA, 23 nt, synthetic construct
  misc_feature 1..23: note = Synthetic
  source 1..23: mol_type = other RNA
ttatagagcaagaacactgtttt

SEQ ID NO: 473 - RNA, 23 nt, synthetic construct
  misc_feature 1..23: note = Synthetic
  source 1..23: mol_type = other RNA
ttatagagcaagaacactgtttt

SEQ ID NO: 474 - RNA, 23 nt, synthetic construct
  misc_feature 1..23: note = Synthetic
  source 1..23: mol_type = other RNA
ttatagagcaagaacactgtttt

SEQ ID NO: 475 - RNA, 23 nt, synthetic construct
  misc_feature 1..23: note = Synthetic
  source 1..23: mol_type = other RNA
ttatagagcaagaacactgtttt

SEQ ID NO: 476 - RNA, 23 nt, synthetic construct
  misc_feature 1..23: note = Synthetic
  source 1..23: mol_type = other RNA
ttatagagcaagaacactgtttt

SEQ ID NO: 477 - RNA, 23 nt, synthetic construct
  misc_feature 1..23: note = Synthetic
  source 1..23: mol_type = other RNA
ttatagagcaagaacactgtttt

SEQ ID NO: 478 - RNA, 23 nt, synthetic construct
  misc_feature 1..23: note = Synthetic
  source 1..23: mol_type = other RNA
ttatagagcaagaacactgtttt

SEQ ID NO: 479 - RNA, 23 nt, synthetic construct
  misc_feature 1..23: note = Synthetic
  source 1..23: mol_type = other RNA
ttatagagcaagaacactgtttt

SEQ ID NO: 480 - RNA, 23 nt, synthetic construct
  misc_feature 1..23: note = Synthetic
  source 1..23: mol_type = other RNA
ttatagagcaagaacactgtttt

SEQ ID NO: 481 - RNA, 23 nt, synthetic construct
  misc_feature 1..23: note = Synthetic
  source 1..23: mol_type = other RNA
ttatagagcaagaacactgtttt

SEQ ID NO: 482 - RNA, 23 nt, synthetic construct
  misc_feature 1..23: note = Synthetic
  source 1..23: mol_type = other RNA
ttatagagcaagaacactgtttt

SEQ ID NO: 483 - RNA, 23 nt, synthetic construct
  misc_feature 1..23: note = Synthetic
  source 1..23: mol_type = other RNA
ttatagagcaagaacactgtttt

SEQ ID NO: 484 - RNA, 23 nt, synthetic construct
  misc_feature 1..23: note = Synthetic
  source 1..23: mol_type = other RNA
ttatagagcaagaacactgtttt

SEQ ID NO: 485 - RNA, 23 nt, synthetic construct
  misc_feature 1..23: note = Synthetic
  source 1..23: mol_type = other RNA
ttatagagcaagaacactgtttt

SEQ ID NO: 486 - RNA, 23 nt, synthetic construct
  misc_feature 1..23: note = Synthetic
  source 1..23: mol_type = other RNA
ttatagagcaagaacactgtttt

SEQ ID NO: 487 - RNA, 23 nt, synthetic construct
  misc_feature 1..23: note = Synthetic
  source 1..23: mol_type = other RNA
ttatagagcaagaacactgtttt

SEQ ID NO: 488 - RNA, 23 nt, synthetic construct
  misc_feature 1..23: note = Synthetic
  source 1..23: mol_type = other RNA
ttatagagcaagaacactgtttt

SEQ ID NO: 489 - RNA, 23 nt, synthetic construct
  misc_feature 1..23: note = Synthetic
  source 1..23: mol_type = other RNA
ttatagagcaagaacactgtttt

SEQ ID NO: 490 - RNA, 23 nt, synthetic construct
  misc_feature 1..23: note = Synthetic
  source 1..23: mol_type = other RNA
ttatagagcaagaacactgtttt

SEQ ID NO: 491 - RNA, 23 nt, synthetic construct
  misc_feature 1..23: note = Synthetic
  source 1..23: mol_type = other RNA
ttatagagcaagaacactgtttt

SEQ ID NO: 492 - RNA, 23 nt, synthetic construct
  misc_feature 1..23: note = Synthetic
  source 1..23: mol_type = other RNA
ttatagagcaagaacactgtttt

SEQ ID NO: 493 - RNA, 23 nt, synthetic construct
  misc_feature 1..23: note = Synthetic
  source 1..23: mol_type = other RNA
ttatagagcaagaacactgtttt

SEQ ID NO: 494 - RNA, 23 nt, synthetic construct
  misc_feature 1..23: note = Synthetic
  source 1..23: mol_type = other RNA
ttatagagcaagaacactgtttt

SEQ ID NO: 495 - RNA, 23 nt, synthetic construct
  misc_feature 1..23: note = Synthetic
  source 1..23: mol_type = other RNA
ttatagagcaagaacactgtttt

SEQ ID NO: 496 - RNA, 23 nt, synthetic construct
  misc_feature 1..23: note = Synthetic
  source 1..23: mol_type = other RNA
ttatagagcaagaacactgtttt

SEQ ID NO: 497 - RNA, 23 nt, synthetic construct
  misc_feature 1..23: note = Synthetic
  source 1..23: mol_type = other RNA
ttatagagcaagaacactgtttt

SEQ ID NO: 498 - RNA, 23 nt, synthetic construct
  misc_feature 1..23: note = Synthetic
  source 1..23: mol_type = other RNA
ttatagagcaagaacactgtttt

SEQ ID NO: 499 - RNA, 23 nt, synthetic construct
  misc_feature 1..23: note = Synthetic
  source 1..23: mol_type = other RNA
ttatagagcaagaacactgtttt

SEQ ID NO: 500 - RNA, 23 nt, synthetic construct
  misc_feature 1..23: note = Synthetic
  source 1..23: mol_type = other RNA
ttatagagcaagaacactgtttt

SEQ ID NO: 501 - RNA, 23 nt, synthetic construct
  misc_feature 1..23: note = Synthetic
  source 1..23: mol_type = other RNA
ttatagagcaagaacactgtttt

SEQ ID NO: 502 - RNA, 23 nt, synthetic construct
  misc_feature 1..23: note = Synthetic
  source 1..23: mol_type = other RNA
ttatagagcaagaacactgtttt

SEQ ID NO: 503 - RNA, 23 nt, synthetic construct
  misc_feature 1..23: note = Synthetic
  source 1..23: mol_type = other RNA
ttatagagcaagaacactgtttt

SEQ ID NO: 504 - RNA, 23 nt, synthetic construct
  misc_feature 1..23: note = Synthetic
  source 1..23: mol_type = other RNA
ttatagagcaagaacactgtttt

SEQ ID NO: 505 - RNA, 23 nt, synthetic construct
  misc_feature 1..23: note = Synthetic
  source 1..23: mol_type = other RNA
ttatagagcaagaacactgtttt

SEQ ID NO: 506 - RNA, 23 nt, synthetic construct
  misc_feature 1..23: note = Synthetic
  source 1..23: mol_type = other RNA
ttatagagcaagaacactgtttt

SEQ ID NO: 507 - RNA, 23 nt, synthetic construct
  misc_feature 1..23: note = Synthetic
  source 1..23: mol_type = other RNA
ttatagagcaagaacactgtttt

SEQ ID NO: 508 - RNA, 23 nt, synthetic construct
  misc_feature 1..23: note = Synthetic
  source 1..23: mol_type = other RNA
ttatagagcaagaacactgtttt

SEQ ID NO: 509 - RNA, 23 nt, synthetic construct
  misc_feature 1..23: note = Synthetic
  source 1..23: mol_type = other RNA
ttatagagcaagaacactgtttt

SEQ ID NO: 510 - RNA, 23 nt, synthetic construct
  misc_feature 1..23: note = Synthetic
  source 1..23: mol_type = other RNA
ttatagagcaagaacactgtttt

SEQ ID NO: 511 - RNA, 23 nt, synthetic construct
  misc_feature 1..23: note = Synthetic
  source 1..23: mol_type = other RNA
ttatagagcaagaacactgtttt

SEQ ID NO: 512 - RNA, 23 nt, synthetic construct
  misc_feature 1..23: note = Synthetic
  source 1..23: mol_type = other RNA
ttatagagcaagaacactgtttt

SEQ ID NO: 513 - RNA, 23 nt, synthetic construct
  misc_feature 1..23: note = Synthetic
  source 1..23: mol_type = other RNA
ttatagagcaagaacactgtttt

SEQ ID NO: 514 - RNA, 23 nt, synthetic construct
  misc_feature 1..23: note = Synthetic
  source 1..23: mol_type = other RNA
ttatagagcaagaacactgtttt

SEQ ID NO: 515 - RNA, 23 nt, synthetic construct
  misc_feature 1..23: note = Synthetic
  source 1..23: mol_type = other RNA
ttatagagcaagaacactgtttt

SEQ ID NO: 516 - RNA, 23 nt, synthetic construct
  misc_feature 1..23: note = Synthetic
  source 1..23: mol_type = other RNA
ttatagagcaagaacactgtttt

SEQ ID NO: 517 - RNA, 23 nt, synthetic construct
  misc_feature 1..23: note = Synthetic
  source 1..23: mol_type = other RNA
ttatagagcaagaacactgtttt

SEQ ID NO: 518 - RNA, 23 nt, synthetic construct
  misc_feature 1..23: note = Synthetic
  source 1..23: mol_type = other RNA
ttatagagcaagaacactgtttt

SEQ ID NO: 519 - RNA, 23 nt, synthetic construct
  misc_feature 1..23: note = Synthetic
  source 1..23: mol_type = other RNA
ttatagagcaagaacactgtttt

SEQ ID NO: 520 - RNA, 23 nt, synthetic construct
  misc_feature 1..23: note = Synthetic
  source 1..23: mol_type = other RNA
ttatagagcaagaacactgtttt

SEQ ID NO: 521 - RNA, 23 nt, synthetic construct
  misc_feature 1..23: note = Synthetic
  source 1..23: mol_type = other RNA
ttatagagcaagaacactgtttt

SEQ ID NO: 522 - RNA, 23 nt, synthetic construct
  misc_feature 1..23: note = Synthetic
  source 1..23: mol_type = other RNA
ttatagagcaagaacactgtttt

SEQ ID NO: 523 - RNA, 23 nt, synthetic construct
  misc_feature 1..23: note = Synthetic
  source 1..23: mol_type = other RNA
ttatagagcaagaacactgtttt

SEQ ID NO: 524 - RNA, 23 nt, synthetic construct
  misc_feature 1..23: note = Synthetic
  source 1..23: mol_type = other RNA
ttatagagcaagaacactgtttt

SEQ ID NO: 525 - RNA, 23 nt, synthetic construct
  misc_feature 1..23: note = Synthetic
  source 1..23: mol_type = other RNA
ttatagagcaagaacactgtttt

SEQ ID NO: 526 - RNA, 23 nt, synthetic construct
  misc_feature 1..23: note = Synthetic
  source 1..23: mol_type = other RNA
ttatagagcaagaacactgtttt

SEQ ID NO: 527 - RNA, 23 nt, synthetic construct
  misc_feature 1..23: note = Synthetic
  source 1..23: mol_type = other RNA
ttatagagcaagaacactgtttt

SEQ ID NO: 528 - RNA, 23 nt, synthetic construct
  misc_feature 1..23: note = Synthetic
  source 1..23: mol_type = other RNA
ttatagagcaagaacactgtttt

SEQ ID NO: 529 - RNA, 23 nt, synthetic construct
  misc_feature 1..23: note = Synthetic
  source 1..23: mol_type = other RNA
ttatagagcaagaacactgtttt

SEQ ID NO: 530 - RNA, 23 nt, synthetic construct
  misc_feature 1..23: note = Synthetic
  source 1..23: mol_type = other RNA
ttatagagcaagaacactgtttt

SEQ ID NO: 531 - RNA, 23 nt, synthetic construct
  misc_feature 1..23: note = Synthetic
  source 1..23: mol_type = other RNA
ttatagagcaagaacactgtttt

SEQ ID NO: 532 - RNA, 23 nt, synthetic construct
  misc_feature 1..23: note = Synthetic
  source 1..23: mol_type = other RNA
ttatagagcaagaacactgtttt

SEQ ID NO: 533 - RNA, 23 nt, synthetic construct
  misc_feature 1..23: note = Synthetic
  source 1..23: mol_type = other RNA
ttatagagcaagaacactgtttt

SEQ ID NO: 534 - RNA, 23 nt, synthetic construct
  misc_feature 1..23: note = Synthetic
  source 1..23: mol_type = other RNA
ttatagagcaagaacactgtttt

SEQ ID NO: 535 - RNA, 23 nt, synthetic construct
  misc_feature 1..23: note = Synthetic
  source 1..23: mol_type = other RNA
ttatagagcaagaacactgtttt

SEQ ID NO: 536 - RNA, 23 nt, synthetic construct
  misc_feature 1..23: note = Synthetic
  source 1..23: mol_type = other RNA
ttatagagcaagaacactgtttt

SEQ ID NO: 537 - RNA, 23 nt, synthetic construct
  misc_feature 1..23: note = Synthetic
  source 1..23: mol_type = other RNA
ttatagagcaagaacactgtttt

SEQ ID NO: 538 - RNA, 23 nt, synthetic construct
  misc_feature 1..23: note = Synthetic
  source 1..23: mol_type = other RNA
ttatagagcaagaacactgtttt

SEQ ID NO: 539 - RNA, 23 nt, synthetic construct
  misc_feature 1..23: note = Synthetic
  source 1..23: mol_type = other RNA
ttatagagcaagaacactgtttt

SEQ ID NO: 540 - RNA, 23 nt, synthetic construct
  misc_feature 1..23: note = Synthetic
  source 1..23: mol_type = other RNA
ttatagagcaagaacactgtttt

SEQ ID NO: 541 - RNA, 23 nt, synthetic construct
  misc_feature 1..23: note = Synthetic
  source 1..23: mol_type = other RNA
ttatagagcaagaacactgtttt

SEQ ID NO: 542 - RNA, 23 nt, synthetic construct
  misc_feature 1..23: note = Synthetic
  source 1..23: mol_type = other RNA
ttatagagcaagaacactgtttt

SEQ ID NO: 543 - RNA, 23 nt, synthetic construct
  misc_feature 1..23: note = Synthetic
  source 1..23: mol_type = other RNA
ttatagagcaagaacactgtttt

SEQ ID NO: 544 - RNA, 23 nt, synthetic construct
  misc_feature 1..23: note = Synthetic
  source 1..23: mol_type = other RNA
ttatagagcaagaacactgtttt

SEQ ID NO: 545 - RNA, 23 nt, synthetic construct
  misc_feature 1..23: note = Synthetic
  source 1..23: mol_type = other RNA
ttatagagcaagaacactgtttt

SEQ ID NO: 546 - RNA, 23 nt, synthetic construct
  misc_feature 1..23: note = Synthetic
  source 1..23: mol_type = other RNA
ttatagagcaagaacactgtttt

SEQ ID NO: 547 - RNA, 23 nt, synthetic construct
  misc_feature 1..23: note = Synthetic
  source 1..23: mol_type = other RNA
ttatagagcaagaacactgtttt

SEQ ID NO: 548 - RNA, 23 nt, synthetic construct
  misc_feature 1..23: note = Synthetic
  source 1..23: mol_type = other RNA
ttatagagcaagaacactgtttt

SEQ ID NO: 549 - RNA, 23 nt, synthetic construct
  misc_feature 1..23: note = Synthetic
  source 1..23: mol_type = other RNA
ttatagagcaagaacactgtttt

SEQ ID NO: 550 - RNA, 23 nt, synthetic construct
  misc_feature 1..23: note = Synthetic
  source 1..23: mol_type = other RNA
ttatagagcaagaacactgtttt

SEQ ID NO: 551 - RNA, 23 nt, synthetic construct
  misc_feature 1..23: note = Synthetic
  source 1..23: mol_type = other RNA
ttatagagcaagaacactgtttt

SEQ ID NO: 552 - RNA, 23 nt, synthetic construct
  misc_feature 1..23: note = Synthetic
  source 1..23: mol_type = other RNA
ttatagagcaagaacactgtttt

SEQ ID NO: 553 - RNA, 23 nt, synthetic construct
  misc_feature 1..23: note = Synthetic
  source 1..23: mol_type = other RNA
ttatagagcaagaacactgtttt

SEQ ID NO: 554 - RNA, 23 nt, synthetic construct
  misc_feature 1..23: note = Synthetic
  source 1..23: mol_type = other RNA
ttatagagcaagaacactgtttt

SEQ ID NO: 555 - RNA, 23 nt, synthetic construct
  misc_feature 1..23: note = Synthetic
  source 1..23: mol_type = other RNA
ttatagagcaagaacactgtttt

SEQ ID NO: 556 - RNA, 23 nt, synthetic construct
  misc_feature 1..23: note = Synthetic
  source 1..23: mol_type = other RNA
ttatagagcaagaacactgtttt

SEQ ID NO: 557 - RNA, 23 nt, synthetic construct
  misc_feature 1..23: note = Synthetic
  source 1..23: mol_type = other RNA
ttatagagcaagaacactgtttt

SEQ ID NO: 558 - RNA, 23 nt, synthetic construct
  misc_feature 1..23: note = Synthetic
  source 1..23: mol_type = other RNA
ttatagagcaagaacactgtttt

SEQ ID NO: 559 - RNA, 23 nt, synthetic construct
  misc_feature 1..23: note = Synthetic
  source 1..23: mol_type = other RNA
ttatagagcaagaacactgtttt

SEQ ID NO: 560 - RNA, 23 nt, synthetic construct
  misc_feature 1..23: note = Synthetic
  source 1..23: mol_type = other RNA
ttatagagcaagaacactgtttt

SEQ ID NO: 561 - RNA, 23 nt, synthetic construct
  misc_feature 1..23: note = Synthetic
  source 1..23: mol_type = other RNA
ttatagagcaagaacactgtttt

SEQ ID NO: 562 - RNA, 23 nt, synthetic construct
  misc_feature 1..23: note = Synthetic
  source 1..23: mol_type = other RNA
ttatagagcaagaacactgtttt

SEQ ID NO: 563 - RNA, 23 nt, synthetic construct
  misc_feature 1..23: note = Synthetic
  source 1..23: mol_type = other RNA
ttatagagcaagaacactgtttt

SEQ ID NO: 564 - RNA, 23 nt, synthetic construct
  misc_feature 1..23: note = Synthetic
  source 1..23: mol_type = other RNA
ttatagagcaagaacactgtttt

SEQ ID NO: 565 - RNA, 23 nt, synthetic construct
  misc_feature 1..23: note = Synthetic
  source 1..23: mol_type = other RNA
ttatagagcaagaacactgtttt

SEQ ID NO: 566 - RNA, 23 nt, synthetic construct
  misc_feature 1..23: note = Synthetic
  source 1..23: mol_type = other RNA
ttatagagcaagaacactgtttt

SEQ ID NO: 567 - RNA, 23 nt, synthetic construct
  misc_feature 1..23: note = Synthetic
  source 1..23: mol_type = other RNA
ttatagagcaagaacactgtttt

SEQ ID NO: 568 - RNA, 23 nt, synthetic construct
  misc_feature 1..23: note = Synthetic
  source 1..23: mol_type = other RNA
ttatagagcaagaacactgtttt

SEQ ID NO: 569 - RNA, 23 nt, synthetic construct
  misc_feature 1..23: note = Synthetic
  source 1..23: mol_type = other RNA
ttatagagcaagaacactgtttt

SEQ ID NO: 570 - RNA, 23 nt, synthetic construct
  misc_feature 1..23: note = Synthetic
  source 1..23: mol_type = other RNA
ttatagagcaagaacactgtttt

SEQ ID NO: 571 - RNA, 23 nt, synthetic construct
  misc_feature 1..23: note = Synthetic
  source 1..23: mol_type = other RNA
ttatagagcaagaacactgtttt

SEQ ID NO: 572 - RNA, 23 nt, synthetic construct
  misc_feature 1..23: note = Synthetic
  source 1..23: mol_type = other RNA
ttatagagcaagaacactgtttt

SEQ ID NO: 573 - RNA, 23 nt, synthetic construct
  misc_feature 1..23: note = Synthetic
  source 1..23: mol_type = other RNA
ttatagagcaagaacactgtttt

SEQ ID NO: 574 - RNA, 23 nt, synthetic construct
  misc_feature 1..23: note = Synthetic
  source 1..23: mol_type = other RNA
ttatagagcaagaacactgtttt

SEQ ID NO: 575 - RNA, 23 nt, synthetic construct
  misc_feature 1..23: note = Synthetic
  source 1..23: mol_type = other RNA
ttatagagcaagaacactgtttt

SEQ ID NO: 576 - RNA, 23 nt, synthetic construct
  misc_feature 1..23: note = Synthetic
  source 1..23: mol_type = other RNA
ttatagagcaagaacactgtttt

SEQ ID NO: 577 - RNA, 23 nt, synthetic construct
  misc_feature 1..23: note = Synthetic
  source 1..23: mol_type = other RNA
ttatagagcaagaacactgtttt

SEQ ID NO: 578 - RNA, 23 nt, synthetic construct
  misc_feature 1..23: note = Synthetic
  source 1..23: mol_type = other RNA
ttatagagcaagaacactgtttt

SEQ ID NO: 579 - RNA, 23 nt, synthetic construct
  misc_feature 1..23: note = Synthetic
  source 1..23: mol_type = other RNA
ttatagagcaagaacactgtttt

SEQ ID NO: 580 - RNA, 23 nt, synthetic construct
  misc_feature 1..23: note = Synthetic
  source 1..23: mol_type = other RNA
ttatagagcaagaacactgtttt

SEQ ID NO: 581 - RNA, 23 nt, synthetic construct
  misc_feature 1..23: note = Synthetic
  source 1..23: mol_type = other RNA
ttatagagcaagaacactgtttt

SEQ ID NO: 582 - RNA, 23 nt, synthetic construct
  misc_feature 1..23: note = Synthetic
  source 1..23: mol_type = other RNA
ttatagagcaagaacactgtttt

SEQ ID NO: 583 - RNA, 23 nt, synthetic construct
  misc_feature 1..23: note = Synthetic
  source 1..23: mol_type = other RNA
ttatagagcaagaacactgtttt

SEQ ID NO: 584 - RNA, 23 nt, synthetic construct
  misc_feature 1..23: note = Synthetic
  source 1..23: mol_type = other RNA
ttatagagcaagaacactgtttt

SEQ ID NO: 585 - RNA, 23 nt, synthetic construct
  misc_feature 1..23: note = Synthetic
  source 1..23: mol_type = other RNA
ttatagagcaagaacactgtttt

SEQ ID NO: 586 - RNA, 23 nt, synthetic construct
  misc_feature 1..23: note = Synthetic
  source 1..23: mol_type = other RNA
ttatagagcaagaacactgtttt

SEQ ID NO: 587 - RNA, 23 nt, synthetic construct
  misc_feature 1..23: note = Synthetic
  source 1..23: mol_type = other RNA
ttatagagcaagaacactgtttt

SEQ ID NO: 588 - RNA, 23 nt, synthetic construct
  misc_feature 1..23: note = Synthetic
  source 1..23: mol_type = other RNA
ttatagagcaagaacactgtttt

SEQ ID NO: 589 - RNA, 23 nt, synthetic construct
  misc_feature 1..23: note = Synthetic
  source 1..23: mol_type = other RNA
ttatagagcaagaacactgtttt

SEQ ID NO: 590 - RNA, 23 nt, synthetic construct
  misc_feature 1..23: note = Synthetic
  source 1..23: mol_type = other RNA
ttatagagcaagaacactgtttt

SEQ ID NO: 591 - RNA, 23 nt, synthetic construct
  misc_feature 1..23: note = Synthetic
  source 1..23: mol_type = other RNA
ttatagagcaagaacactgtttt

SEQ ID NO: 592 - RNA, 23 nt, synthetic construct
  misc_feature 1..23: note = Synthetic
  source 1..23: mol_type = other RNA
ttatagagcaagaacactgtttt

SEQ ID NO: 593 - RNA, 23 nt, synthetic construct
  misc_feature 1..23: note = Synthetic
  source 1..23: mol_type = other RNA
ttatagagcaagaacactgtttt

SEQ ID NO: 594 - RNA, 23 nt, synthetic construct
  misc_feature 1..23: note = Synthetic
  source 1..23: mol_type = other RNA
ttatagagcaagaacactgtttt

SEQ ID NO: 595 - RNA, 23 nt, synthetic construct
  misc_feature 1..23: note = Synthetic
  source 1..23: mol_type = other RNA
ttatagagcaagaacactgtttt

SEQ ID NO: 596 - RNA, 23 nt, synthetic construct
  misc_feature 1..23: note = Synthetic
  source 1..23: mol_type = other RNA
ttatagagcaagaacactgtttt

SEQ ID NO: 597 - RNA, 23 nt, synthetic construct
  misc_feature 1..23: note = Synthetic
  source 1..23: mol_type = other RNA
ttatagagcaagaacactgtttt

SEQ ID NO: 598 - RNA, 23 nt, synthetic construct
  misc_feature 1..23: note = Synthetic
  source 1..23: mol_type = other RNA
ttatagagcaagaacactgtttt

SEQ ID NO: 599 - RNA, 23 nt, synthetic construct
  misc_feature 1..23: note = Synthetic
  source 1..23: mol_type = other RNA
ttatagagcaagaacactgtttt

SEQ ID NO: 600 - RNA, 23 nt, synthetic construct
  misc_feature 1..23: note = Synthetic
  source 1..23: mol_type = other RNA
ttatagagcaagaacactgtttt

SEQ ID NO: 601 - RNA, 23 nt, synthetic construct
  misc_feature 1..23: note = Synthetic
  source 1..23: mol_type = other RNA
ttatagagcaagaacactgtttt

SEQ ID NO: 602 - RNA, 23 nt, synthetic construct
  misc_feature 1..23: note = Synthetic
  source 1..23: mol_type = other RNA
ttatagagcaagaacactgtttt

SEQ ID NO: 603 - RNA, 23 nt, synthetic construct
  misc_feature 1..23: note = Synthetic
  source 1..23: mol_type = other RNA
ttatagagcaagaacactgtttt

SEQ ID NO: 604 - RNA, 23 nt, synthetic construct
  misc_feature 1..23: note = Synthetic
  source 1..23: mol_type = other RNA
ttatagagcaagaacactgtttt

SEQ ID NO: 605 - RNA, 23 nt, synthetic construct
  misc_feature 1..23: note = Synthetic
  source 1..23: mol_type = other RNA
ttatagagcaagaacactgtttt

SEQ ID NO: 606 - RNA, 23 nt, synthetic construct
  misc_feature 1..23: note = Synthetic
  source 1..23: mol_type = other RNA
ttatagagcaagaacactgtttt

SEQ ID NO: 607 - RNA, 21 nt, synthetic construct
  misc_feature 1..21: note = Synthetic
  source 1..21: mol_type = other RNA
aacagtgttcttgctctataa

SEQ ID NO: 608 - RNA, 21 nt, synthetic construct
  misc_feature 1..21: note = Synthetic
  source 1..21: mol_type = other RNA
aacagtgttcttgctctataa

SEQ ID NO: 609 - RNA, 23 nt, synthetic construct
  misc_feature 1..23: note = Synthetic
  source 1..23: mol_type = other RNA
ttatagagcaagaacactgtttt

SEQ ID NO: 610 - RNA, 23 nt, synthetic construct
  misc_feature 1..23: note = Synthetic
  source 1..23: mol_type = other RNA
ttatagagcaagaacactgtttt

SEQ ID NO: 611 - RNA, 23 nt, synthetic construct
  misc_feature 1..23: note = Synthetic
  source 1..23: mol_type = other RNA
ttatagagcaagaacactgtttt

SEQ ID NO: 612 - RNA, 23 nt, synthetic construct
  misc_feature 1..23: note = Synthetic
  source 1..23: mol_type = other RNA
ttatagagcaagaacactgtttt

SEQ ID NO: 613 - RNA, 23 nt, synthetic construct
  misc_feature 1..23: note = Synthetic
  source 1..23: mol_type = other RNA
ttatagagcaagaacactgtttt

SEQ ID NO: 614 - RNA, 23 nt, synthetic construct
  misc_feature 1..23: note = Synthetic
  source 1..23: mol_type = other RNA
ttatagagcaagaacactgtttt

SEQ ID NO: 615 - RNA, 23 nt, synthetic construct
  misc_feature 1..23: note = Synthetic
  source 1..23: mol_type = other RNA
ttatagagcaagaacactgtttt

SEQ ID NO: 616 - RNA, 23 nt, synthetic construct
  misc_feature 1..23: note = Synthetic
  source 1..23: mol_type = other RNA
ttatagagcaagaacactgtttt

SEQ ID NO: 617 - RNA, 23 nt, synthetic construct
  misc_feature 1..23: note = Synthetic
  source 1..23: mol_type = other RNA
ttatagagcaagaacactgtttt

SEQ ID NO: 618 - RNA, 23 nt, synthetic construct
  misc_feature 1..23: note = Synthetic
  source 1..23: mol_type = other RNA
ttatagagcaagaacactgtttt

SEQ ID NO: 619 - RNA, 23 nt, synthetic construct
  misc_feature 1..23: note = Synthetic
  source 1..23: mol_type = other RNA
ttatagagcaagaacactgtttt

SEQ ID NO: 620 - RNA, 23 nt, synthetic construct
  misc_feature 1..23: note = Synthetic
  source 1..23: mol_type = other RNA
ttatagagcaagaacactgtttt

SEQ ID NO: 621 - RNA, 23 nt, synthetic construct
  misc_feature 1..23: note = Synthetic
  source 1..23: mol_type = other RNA
ttatagagcaagaacactgtttt

SEQ ID NO: 622 - RNA, 23 nt, synthetic construct
  misc_feature 1..23: note = Synthetic
  source 1..23: mol_type = other RNA
ttatagagcaagaacactgtttt

SEQ ID NO: 623 - RNA, 23 nt, synthetic construct
  misc_feature 1..23: note = Synthetic
  source 1..23: mol_type = other RNA
ttatagagcaagaacactgtttt

SEQ ID NO: 624 - RNA, 23 nt, synthetic construct
  misc_feature 1..23: note = Synthetic
  source 1..23: mol_type = other RNA
ttatagagcaagaacactgtttt

SEQ ID NO: 625 - RNA, 23 nt, synthetic construct
  misc_feature 1..23: note = Synthetic
  source 1..23: mol_type = other RNA
ttatagagcaagaacactgtttt

SEQ ID NO: 626 - RNA, 23 nt, synthetic construct
  misc_feature 1..23: note = Synthetic
  source 1..23: mol_type = other RNA
ttatagagcaagaacactgtttt

SEQ ID NO: 627 - RNA, 23 nt, synthetic construct
  misc_feature 1..23: note = Synthetic
  source 1..23: mol_type = other RNA
ttatagagcaagaacactgtttt

SEQ ID NO: 628 - RNA, 23 nt, synthetic construct
  misc_feature 1..23: note = Synthetic
  source 1..23: mol_type = other RNA
ttatagagcaagaacactgtttt

SEQ ID NO: 629 - RNA, 23 nt, synthetic construct
  misc_feature 1..23: note = Synthetic
  source 1..23: mol_type = other RNA
ttatagagcaagaacactgtttt

SEQ ID NO: 630 - RNA, 23 nt, synthetic construct
  misc_feature 1..23: note = Synthetic
  source 1..23: mol_type = other RNA
ttatagagcaagaacactgtttt

SEQ ID NO: 631 - RNA, 23 nt, synthetic construct
  misc_feature 1..23: note = Synthetic
  source 1..23: mol_type = other RNA
ttatagagcaagaacactgtttt

SEQ ID NO: 632 - RNA, 23 nt, synthetic construct
  misc_feature 1..23: note = Synthetic
  source 1..23: mol_type = other RNA
ttatagagcaagaacactgtttt

SEQ ID NO: 633 - RNA, 23 nt, synthetic construct
  misc_feature 1..23: note = Synthetic
  source 1..23: mol_type = other RNA
ttatagagcaagaacactgtttt

SEQ ID NO: 634 - RNA, 23 nt, synthetic construct
  misc_feature 1..23: note = Synthetic
  source 1..23: mol_type = other RNA
ttatagagcaagaacactgtttt

SEQ ID NO: 635 - RNA, 23 nt, synthetic construct
  misc_feature 1..23: note = Synthetic
  source 1..23: mol_type = other RNA
ttatagagcaagaacactgtttt

SEQ ID NO: 636 - RNA, 23 nt, synthetic construct
  misc_feature 1..23: note = Synthetic
  source 1..23: mol_type = other RNA
ttatagagcaagaacactgtttt

SEQ ID NO: 637 - RNA, 23 nt, synthetic construct
  misc_feature 1..23: note = Synthetic
  source 1..23: mol_type = other RNA
ttatagagcaagaacactgtttt

SEQ ID NO: 638 - RNA, 23 nt, synthetic construct
  misc_feature 1..23: note = Synthetic
  source 1..23: mol_type = other RNA
ttatagagcaagaacactgtttt

SEQ ID NO: 639 - RNA, 23 nt, synthetic construct
  misc_feature 1..23: note = Synthetic
  source 1..23: mol_type = other RNA
ttatagagcaagaacactgtttt

SEQ ID NO: 640 - RNA, 23 nt, synthetic construct
  misc_feature 1..23: note = Synthetic
  source 1..23: mol_type = other RNA
ttatagagcaagaacactgtttt

SEQ ID NO: 641 - RNA, 23 nt, synthetic construct
  misc_feature 1..23: note = Synthetic
  source 1..23: mol_type = other RNA
ttatagagcaagaacactgtttt

SEQ ID NO: 642 - RNA, 23 nt, synthetic construct
  misc_feature 1..23: note = Synthetic
  source 1..23: mol_type = other RNA
tccttccctgaaggttcctcctt

SEQ ID NO: 643 - RNA, 23 nt, synthetic construct
  misc_feature 1..23: note = Synthetic
  source 1..23: mol_type = other RNA
tccttccctgaaggttcctcctt

SEQ ID NO: 644 - RNA, 21 nt, synthetic construct
  misc_feature 1..21: note = Synthetic
  source 1..21: mol_type = other RNA
ggaggaaccttcagggaagga

SEQ ID NO: 645 - RNA, 23 nt, synthetic construct
  misc_feature 1..23: note = Synthetic
  source 1..23: mol_type = other RNA
ttatagagcaagaacactgtttt

SEQ ID NO: 646 - RNA, 23 nt, synthetic construct
  misc_feature 1..23: note = Synthetic
  source 1..23: mol_type = other RNA
tccttccctgaaggttcctcctt

SEQ ID NO: 647 - RNA, 21 nt, synthetic construct
  misc_feature 1..21: note = Synthetic
  source 1..21: mol_type = other RNA
ggaggaaccttcagggaagga

SEQ ID NO: 648 - RNA, 23 nt, synthetic construct
  misc_feature 1..23: note = Synthetic
  source 1..23: mol_type = other RNA
ttatagagcaagaacactgtttt

SEQ ID NO: 649 - RNA, 23 nt, synthetic construct
  misc_feature 1..23: note = Synthetic
  source 1..23: mol_type = other RNA
ttatagagcaagaacactgtttt

SEQ ID NO: 650 - RNA, 23 nt, synthetic construct
  misc_feature 1..23: note = Synthetic
  source 1..23: mol_type = other RNA
ttatagagcaagaacactgtttt

SEQ ID NO: 651 - RNA, 23 nt, synthetic construct
  misc_feature 1..23: note = Synthetic
  source 1..23: mol_type = other RNA
ttatagagcaagaacactgtttt

SEQ ID NO: 652 - RNA, 23 nt, synthetic construct
  misc_feature 1..23: note = Synthetic
  source 1..23: mol_type = other RNA
ttatagagcaagaacactgtttt

SEQ ID NO: 653 - RNA, 23 nt, synthetic construct
  misc_feature 1..23: note = Synthetic
  source 1..23: mol_type = other RNA
ttatagagcaagaacactgtttt

SEQ ID NO: 654 - RNA, 23 nt, synthetic construct
  misc_feature 1..23: note = Synthetic
  source 1..23: mol_type = other RNA
ttatagagcaagaacactgtttt

SEQ ID NO: 655 - RNA, 23 nt, synthetic construct
  misc_feature 1..23: note = Synthetic
  source 1..23: mol_type = other RNA
ttatagagcaagaacactgtttt

SEQ ID NO: 656 - RNA, 23 nt, synthetic construct
  misc_feature 1..23: note = Synthetic
  source 1..23: mol_type = other RNA
ttatagagcaagaacactgtttt

SEQ ID NO: 657 - RNA, 23 nt, synthetic construct
  misc_feature 1..23: note = Synthetic
  source 1..23: mol_type = other RNA
ttatagagcaagaacactgtttt

SEQ ID NO: 658 - RNA, 23 nt, synthetic construct
  misc_feature 1..23: note = Synthetic
  source 1..23: mol_type = other RNA
ttatagagcaagaacactgtttt

SEQ ID NO: 659 - RNA, 23 nt, synthetic construct
  misc_feature 1..23: note = Synthetic
  source 1..23: mol_type = other RNA
ttatagagcaagaacactgtttt

SEQ ID NO: 660 - RNA, 23 nt, synthetic construct
  misc_feature 1..23: note = Synthetic
  source 1..23: mol_type = other RNA
ttatagagcaagaacactgtttt

SEQ ID NO: 661 - RNA, 23 nt, synthetic construct
  misc_feature 1..23: note = Synthetic
  source 1..23: mol_type = other RNA
ttatagagcaagaacactgtttt

SEQ ID NO: 662 - RNA, 23 nt, synthetic construct
  misc_feature 1..23: note = Synthetic
  source 1..23: mol_type = other RNA
ttatagagcaagaacactgtttt

SEQ ID NO: 663 - RNA, 23 nt, synthetic construct
  misc_feature 1..23: note = Synthetic
  source 1..23: mol_type = other RNA
ttatagagcaagaacactgtttt

SEQ ID NO: 664 - RNA, 23 nt, synthetic construct
  misc_feature 1..23: note = Synthetic
  source 1..23: mol_type = other RNA
ttatagagcaagaacactgtttt

SEQ ID NO: 665 - RNA, 23 nt, synthetic construct
  misc_feature 1..23: note = Synthetic
  source 1..23: mol_type = other RNA
ttatagagcaagaacactgtttt

SEQ ID NO: 666 - RNA, 23 nt, synthetic construct
  misc_feature 1..23: note = Synthetic
  source 1..23: mol_type = other RNA
ttatagagcaagaacactgtttt

SEQ ID NO: 667 - RNA, 23 nt, synthetic construct
  misc_feature 1..23: note = Synthetic
  source 1..23: mol_type = other RNA
ttatagagcaagaacactgtttt

SEQ ID NO: 668 - RNA, 23 nt, synthetic construct
  misc_feature 1..23: note = Synthetic
  source 1..23: mol_type = other RNA
ttatagagcaagaacactgtttt

SEQ ID NO: 669 - RNA, 23 nt, synthetic construct
  misc_feature 1..23: note = Synthetic
  source 1..23: mol_type = other RNA
ttatagagcaagaacactgtttt

SEQ ID NO: 670 - RNA, 23 nt, synthetic construct
  misc_feature 1..23: note = Synthetic
  source 1..23: mol_type = other RNA
ttatagagcaagaacactgtttt

SEQ ID NO: 671 - RNA, 23 nt, synthetic construct
  misc_feature 1..23: note = Synthetic
  source 1..23: mol_type = other RNA
ttatagagcaagaacactgtttt

SEQ ID NO: 672 - RNA, 23 nt, synthetic construct
  misc_feature 1..23: note = Synthetic
  source 1..23: mol_type = other RNA
ttatagagcaagaacactgtttt

SEQ ID NO: 673 - RNA, 23 nt, synthetic construct
  misc_feature 1..23: note = Synthetic
  source 1..23: mol_type = other RNA
ttatagagcaagaacactgtttt

SEQ ID NO: 674 - RNA, 23 nt, synthetic construct
  misc_feature 1..23: note = Synthetic
  source 1..23: mol_type = other RNA
ttatagagcaagaacactgtttt

SEQ ID NO: 675 - RNA, 23 nt, synthetic construct
  misc_feature 1..23: note = Synthetic
  source 1..23: mol_type = other RNA
ttatagagcaagaacactgtttt

SEQ ID NO: 676 - RNA, 23 nt, synthetic construct
  misc_feature 1..23: note = Synthetic
  source 1..23: mol_type = other RNA
ttatagagcaagaacactgtttt

SEQ ID NO: 677 - RNA, 23 nt, synthetic construct
  misc_feature 1..23: note = Synthetic
  source 1..23: mol_type = other RNA
ttatagagcaagaacactgtttt

SEQ ID NO: 678 - RNA, 23 nt, synthetic construct
  misc_feature 1..23: note = Synthetic
  source 1..23: mol_type = other RNA
ttatagagcaagaacactgtttt

SEQ ID NO: 679 - RNA, 23 nt, synthetic construct
  misc_feature 1..23: note = Synthetic
  source 1..23: mol_type = other RNA
ttatagagcaagaacactgtttt

SEQ ID NO: 680 - RNA, 23 nt, synthetic construct
  misc_feature 1..23: note = Synthetic
  source 1..23: mol_type = other RNA
tccttccctgaaggttcctcctt

SEQ ID NO: 681 - RNA, 23 nt, synthetic construct
  misc_feature 1..23: note = Synthetic
  source 1..23: mol_type = other RNA
tccttccctgaaggttcctcctt

SEQ ID NO: 682 - RNA, 21 nt, synthetic construct
  misc_feature 1..21: note = Synthetic
  source 1..21: mol_type = other RNA
ggaggaaccttcagggaagga

SEQ ID NO: 683 - RNA, 23 nt, synthetic construct
  misc_feature 1..23: note = Synthetic
  source 1..23: mol_type = other RNA
tccttccctgaaggttcctcctt

SEQ ID NO: 684 - RNA, 21 nt, synthetic construct
  misc_feature 1..21: note = Synthetic
  source 1..21: mol_type = other RNA
ggaggaaccttcagggaagga

SEQ ID NO: 685 - RNA, 23 nt, synthetic construct
  misc_feature 1..23: note = Synthetic
  source 1..23: mol_type = other RNA
tccttccctgaaggttcctcctt

SEQ ID NO: 686 - RNA, 21 nt, synthetic construct
  misc_feature 1..21: note = Synthetic
  source 1..21: mol_type = other RNA
ggaggaaccttcagggaagga

SEQ ID NO: 687 - RNA, 23 nt, synthetic construct
  misc_feature 1..23: note = Synthetic
  source 1..23: mol_type = other RNA
ttatagagcaagaacactgtttt

SEQ ID NO: 688 - RNA, 23 nt, synthetic construct
  misc_feature 1..23: note = Synthetic
  source 1..23: mol_type = other RNA
ttatagagcaagaacactgtttt

SEQ ID NO: 689 - RNA, 23 nt, synthetic construct
  misc_feature 1..23: note = Synthetic
  source 1..23: mol_type = other RNA
ttatagagcaagaacactgtttt

SEQ ID NO: 690 - RNA, 23 nt, synthetic construct
  misc_feature 1..23: note = Synthetic
  source 1..23: mol_type = other RNA
ttatagagcaagaacactgtttt

SEQ ID NO: 691 - RNA, 23 nt, synthetic construct
  misc_feature 1..23: note = Synthetic
  source 1..23: mol_type = other RNA
ttatagagcaagaacactgtttt

SEQ ID NO: 692 - RNA, 23 nt, synthetic construct
  misc_feature 1..23: note = Synthetic
  source 1..23: mol_type = other RNA
ttatagagcaagaacactgtttt

SEQ ID NO: 693 - RNA, 23 nt, synthetic construct
  misc_feature 1..23: note = Synthetic
  source 1..23: mol_type = other RNA
ttatagagcaagaacactgtttt

SEQ ID NO: 694 - RNA, 23 nt, synthetic construct
  misc_feature 1..23: note = Synthetic
  source 1..23: mol_type = other RNA
ttatagagcaagaacactgtttt

SEQ ID NO: 695 - RNA, 23 nt, synthetic construct
  misc_feature 1..23: note = Synthetic
  source 1..23: mol_type = other RNA
ttatagagcaagaacactgtttt

SEQ ID NO: 696 - RNA, 23 nt, synthetic construct
  misc_feature 1..23: note = Synthetic
  source 1..23: mol_type = other RNA
ttatagagcaagaacactgtttt

SEQ ID NO: 697 - RNA, 23 nt, synthetic construct
  misc_feature 1..23: note = Synthetic
  source 1..23: mol_type = other RNA
ttatagagcaagaacactgtttt

SEQ ID NO: 698 - RNA, 23 nt, synthetic construct
  misc_feature 1..23: note = Synthetic
  source 1..23: mol_type = other RNA
ttatagagcaagaacactgtttt

SEQ ID NO: 699 - RNA, 23 nt, synthetic construct
  misc_feature 1..23: note = Synthetic
  source 1..23: mol_type = other RNA
ttatagagcaagaacactgtttt

SEQ ID NO: 700 - RNA, 23 nt, synthetic construct
  misc_feature 1..23: note = Synthetic
  source 1..23: mol_type = other RNA
ttatagagcaagaacactgtttt

SEQ ID NO: 701 - RNA, 23 nt, synthetic construct
  misc_feature 1..23: note = Synthetic
  source 1..23: mol_type = other RNA
ttatagagcaagaacactgtttt

SEQ ID NO: 702 - RNA, 23 nt, synthetic construct
  misc_feature 1..23: note = Synthetic
  source 1..23: mol_type = other RNA
ttatagagcaagaacactgtttt

SEQ ID NO: 703 - RNA, 23 nt, synthetic construct
  misc_feature 1..23: note = Synthetic
  source 1..23: mol_type = other RNA
ttatagagcaagaacactgtttt

SEQ ID NO: 704 - RNA, 23 nt, synthetic construct
  misc_feature 1..23: note = Synthetic
  source 1..23: mol_type = other RNA
ttatagagcaagaacactgtttt

SEQ ID NO: 705 - RNA, 23 nt, synthetic construct
  misc_feature 1..23: note = Synthetic
  source 1..23: mol_type = other RNA
ttatagagcaagaacactgtttt

SEQ ID NO: 706 - RNA, 23 nt, synthetic construct
  misc_feature 1..23: note = Synthetic
  source 1..23: mol_type = other RNA
ttatagagcaagaacactgtttt

SEQ ID NO: 707 - RNA, 23 nt, synthetic construct
  misc_feature 1..23: note = Synthetic
  source 1..23: mol_type = other RNA
ttatagagcaagaacactgtttt

SEQ ID NO: 708 - RNA, 23 nt, synthetic construct
  misc_feature 1..23: note = Synthetic
  source 1..23: mol_type = other RNA
ttatagagcaagaacactgtttt

SEQ ID NO: 709 - RNA, 23 nt, synthetic construct
  misc_feature 1..23: note = Synthetic
  source 1..23: mol_type = other RNA
ttatagagcaagaacactgtttt

SEQ ID NO: 710 - RNA, 23 nt, synthetic construct
  misc_feature 1..23: note = Synthetic
  source 1..23: mol_type = other RNA
ttatagagcaagaacactgtttt

SEQ ID NO: 711 - RNA, 23 nt, synthetic construct
  misc_feature 1..23: note = Synthetic
  source 1..23: mol_type = other RNA
ttatagagcaagaacactgtttt

SEQ ID NO: 712 - RNA, 23 nt, synthetic construct
  misc_feature 1..23: note = Synthetic
  source 1..23: mol_type = other RNA
ttatagagcaagaacactgtttt

SEQ ID NO: 713 - RNA, 23 nt, synthetic construct
  misc_feature 1..23: note = Synthetic
  source 1..23: mol_type = other RNA
ttatagagcaagaacactgtttt

SEQ ID NO: 714 - RNA, 23 nt, synthetic construct
  misc_feature 1..23: note = Synthetic
  source 1..23: mol_type = other RNA
ttatagagcaagaacactgtttt

SEQ ID NO: 715 - RNA, 23 nt, synthetic construct
  misc_feature 1..23: note = Synthetic
  source 1..23: mol_type = other RNA
ttatagagcaagaacactgtttt

SEQ ID NO: 716 - RNA, 23 nt, synthetic construct
  misc_feature 1..23: note = Synthetic
  source 1..23: mol_type = other RNA
ttatagagcaagaacactgtttt

SEQ ID NO: 717 - RNA, 23 nt, synthetic construct
  misc_feature 1..23: note = Synthetic
  source 1..23: mol_type = other RNA
ttatagagcaagaacactgtttt

SEQ ID NO: 718 - RNA, 23 nt, synthetic construct
  misc_feature 1..23: note = Synthetic
  source 1..23: mol_type = other RNA
ttatagagcaagaacactgtttt

SEQ ID NO: 719 - RNA, 23 nt, synthetic construct
  misc_feature 1..23: note = Synthetic
  source 1..23: mol_type = other RNA
ttatagagcaagaacactgtttt

SEQ ID NO: 720 - RNA, 23 nt, synthetic construct
  misc_feature 1..23: note = Synthetic
  source 1..23: mol_type = other RNA
ttatagagcaagaacactgtttt

SEQ ID NO: 721 - RNA, 23 nt, synthetic construct
  misc_feature 1..23: note = Synthetic
  source 1..23: mol_type = other RNA
ttatagagcaagaacactgtttt

SEQ ID NO: 722 - RNA, 23 nt, synthetic construct
  misc_feature 1..23: note = Synthetic
  source 1..23: mol_type = other RNA
ttatagagcaagaacactgtttt

SEQ ID NO: 723 - RNA, 23 nt, synthetic construct
  misc_feature 1..23: note = Synthetic
  source 1..23: mol_type = other RNA
ttatagagcaagaacactgtttt

SEQ ID NO: 724 - RNA, 23 nt, synthetic construct
  misc_feature 1..23: note = Synthetic
  source 1..23: mol_type = other RNA
ttatagagcaagaacactgtttt

SEQ ID NO: 725 - RNA, 23 nt, synthetic construct
  misc_feature 1..23: note = Synthetic
  source 1..23: mol_type = other RNA
ttatagagcaagaacactgtttt

SEQ ID NO: 726 - RNA, 23 nt, synthetic construct
  misc_feature 1..23: note = Synthetic
  source 1..23: mol_type = other RNA
ttatagagcaagaacactgtttt

SEQ ID NO: 727 - RNA, 23 nt, synthetic construct
  misc_feature 1..23: note = Synthetic
  source 1..23: mol_type = other RNA
ttatagagcaagaacactgtttt

SEQ ID NO: 728 - RNA, 23 nt, synthetic construct
  misc_feature 1..23: note = Synthetic
  source 1..23: mol_type = other RNA
ttatagagcaagaacactgtttt

SEQ ID NO: 729 - RNA, 23 nt, synthetic construct
  misc_feature 1..23: note = Synthetic
  source 1..23: mol_type = other RNA
ttatagagcaagaacactgtttt

SEQ ID NO: 730 - RNA, 23 nt, synthetic construct
  misc_feature 1..23: note = Synthetic
  source 1..23: mol_type = other RNA
ttatagagcaagaacactgtttt

SEQ ID NO: 731 - RNA, 23 nt, synthetic construct
  misc_feature 1..23: note = Synthetic
  source 1..23: mol_type = other RNA
ttatagagcaagaacactgtttt

SEQ ID NO: 732 - RNA, 21 nt, synthetic construct
  misc_feature 1..21: note = Synthetic
  source 1..21: mol_type = other RNA
aacagtgttcttgctctataa

SEQ ID NO: 733 - RNA, 21 nt, synthetic construct
  misc_feature 1..21: note = Synthetic
  source 1..21: mol_type = other RNA
aacagtgttcttgctctataa

SEQ ID NO: 734 - DNA, 21 bp, synthetic construct
  misc_feature 1..21: note = Synthetic
  source 1..21: mol_type = other DNA
caaactttgctttccctggtt

SEQ ID NO: 735 - DNA, 20 bp, synthetic construct
  misc_feature 1..20: note = Synthetic
  source 1..20: mol_type = other DNA
tggcctgtatccaacacttc

SEQ ID NO: 736 - DNA, 24 bp, synthetic construct
  misc_feature 1..24: note = Synthetic
  source 1..24: mol_type = other DNA
accagcaagcttgcaaccttaacc